(19) United States
(12) Patent Application Publication
(10) Pub. No.: US 2025/0295809 A1
(43) Pub. Date: Sep. 25, 2025

(54) COMPOSITIONS AND METHODS FOR THE TREATMENT OF HER2 POSITIVE CANCER

(71) Applicant: VOYAGER THERAPEUTICS, INC., Lexington, MA (US)

(72) Inventors: Dan Richard LAKS, Cambridge, MA (US); Samuel Aaron HASSON, Arlington, MA (US); Kelly BALES, Lincoln, MA (US); Kenny CHEN, Watertown, MA (US); Usman Yasin HAMEEDI, Malden, MA (US); Xiaoqin REN, North Attleboro, MA (US); Ishan Sanjeev SHAH, Andover, MA (US); Mathieu Emmanuel NONNENMACHER, Boston, MA (US); Tyler Christopher MOYER, Boston, MA (US); Jiangyu LI, Watertown, MA (US)

(21) Appl. No.: 18/862,369

(22) PCT Filed: May 11, 2023
(86) PCT No.: PCT/US2023/066905

§ 371 Date: Nov. 1, 2024

Related U.S. Application Data
(60) Provisional application No. 63/341,857, filed on May 13, 2022.

(51) Int. Cl. A61K 48/00; A61K 45/06; A61P 35/00; C07K 14/005; C07K 16/32; C12N 15/86

(52) U.S. Cl. CPC A61K 48/005; A61K 45/06; A61K 48/0075; A61P 35/00; C07K 14/005; C07K 16/32; C12N 15/86; C07K 2317/31; C07K 2317/52; C07K 2317/565; C07K 2317/71; C12N 2750/14122; C12N 2750/14143; C12N 2750/14151; C12N 2830/50

(57) ABSTRACT

The disclosure provides compositions and methods for the preparation, manufacture and use of an adeno-associated virus (AAV) particle for the vectorized delivery of an antibody molecule that binds to HER2.

### RELATED APPLICATIONS

This application claims priority to U.S. Provisional Application No. 63/341,857 filed on May 13, 2022; the entire contents of which are hereby incorporated by reference in its entirety.

### SEQUENCE LISTING

This instant application contains a Sequence Listing which has been submitted electronically in XML format and is hereby incorporated by reference in its entirety. Said XML copy, created on Apr. 20, 2023, is named V2071-3003PCT_SL.xml and is 5,053,643 bytes in size.

### FIELD OF THE DISCLOSURE

The present disclosure relates generally to compositions and methods for vectorized delivery (VAD) of an antibody molecule, e.g., an antibody molecule that binds to HER2.

### BACKGROUND

Breast cancer is the most common form of cancer and the leading cause of cancer death in women worldwide. Today the systemic treatment of breast cancer offers three major different treatment modalities and the applicability of these different treatment options is substantially dependent on the receptor status of the patient (Bernard-Marty et al., “Facts and controversies in systemic treatment of metastatic breast cancer” Oncologist 9:617-632 (2004)). Endocrine and biological therapy requires the presence of the respective receptors on the cancer cells, whereas cytotoxic chemotherapy is independent of those specified receptors.

Although HER2 receptors are found overexpressed in various cancers, many of the cancer therapies targeting HER2 have been developed for breast cancer. HER2 overexpression and/or amplification have been detected in 10%-34% of invasive breast cancers and correlate with poor prognosis, and poor response to chemotherapy and endocrine therapy. Amplification and/or overexpression of HER2 may play a role in the occurrence or progression of brain metastases. The incidence of brain metastasis in patients with metastatic breast cancer varies from 10 to 15% and these rates increase up to 30-50% in patients with HER2+ breast cancer (Aversa et al., “Metastatic breast cancer subtypes and central nervous system metastases” Breast. 23: 623-628 (2014); Kennecke et al., “Metastatic behavior of breast cancer subtypes” J. Clin. Oncol. 28: 3271-3277 (2010)).

Brain metastases accompanying breast cancer are associated with particularly poor prognosis. Brain metastases seriously affect quality of life and are relatively resistant to systemic therapies. Though the biological basis is not yet fully understood, patients with HER2-positive breast cancer are at a particularly high risk of brain metastases. Currently, the standard component of systemic therapy in HER2-positive breast cancer patients is trastuzumab, a monoclonal antibody against the extracellular domain of the HER2 receptor. However, due to a high molecular weight (approx. 145,000 Da), and physical and chemical properties, trastuzumab does not cross the blood-brain barrier and is ineffective in preventing and treating brain metastases. In highlighting the potential impact of a therapy addressing brain metastases arising from HER2+ breast cancer, one study found that approximately 50% of their cohort of 122 women ultimately died of cerebral progression (Bendell et al., “Central nervous system metastases in women who receive trastuzumab-based therapy for metastatic breast carcinoma” Cancer 97(12):2972-2977 (2003)). Therefore, targeting the CNS progression of the tumors is a major unmet need tied to the shortcoming of current therapies (biodistribution, efficacy) and the patient outcomes when the metastases are unmitigated. As such, there is a medical need for improved compositions and methods of prevention, treatment, and diagnosis for diseases associated with overexpression of HER2, such as metastatic breast cancer.

### SUMMARY

The present disclosure pertains, at least in part, to compositions and methods for the treatment of a disease or disorder associated with HER2 over-expression, e.g., HER2-positive, HER2-amplified and/or HER2-mutated cancer, including modulating the activity of HER2 (e.g., inhibiting HER2 signaling), inducing antibody-dependent cellular cytotoxicity (ADCC), and/or delivery, e.g., vectorized delivery, of an antibody molecule that binds to HER2, e.g., an anti-HER2 antibody molecule described herein. In some embodiments, the level of HER2-mediated cell signaling and tumor growth, is reduced or inhibited using an isolated, e.g., recombinant. AAV particle comprising a genetic element encoding an anti-HER2 antibody molecule, e.g., an anti-HER2 antibody molecule described herein. In some embodiments, the inhibition of HER2 dimerization, downregulation of HER2, and antibody-dependent cell-mediated cytotoxicity is increased using an isolated, e.g., recombinant, AAV particle comprising a genetic element encoding an anti-HER2 antibody molecule, e.g., an anti-HER2 antibody molecule described herein. Such inhibition and/or degradation can be useful in treating disorders related to over-expression of HER2, such as cancer.

Accordingly, in one aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises an amino acid sequence having the following formula: [N1]-[N2]-[N3], wherein: (i) optionally [N1] comprises X1, X2, and X3, wherein at least one of X1, X2, or X3 is G; (ii) [N2] comprises the amino acid sequence of SPH; (ii) [N3] comprises X4, X5, and X6, wherein at least one of X4, X5, or X6 is a basic amino acid, e.g., a K or R. In some embodiments, position X4 of [N3] is K. In some embodiments, position X5 of [N3] is K. In some embodiments, [N3] is or comprises SKA. In some embodiments [N3] is or comprises KSG. In some embodiments, [N2]-[N3] is present immediately subsequent to position 455, numbered according to SEQ ID NO: 138, 981 or 982. In some embodiments, [N1] is present immediately subsequent to position 452, relative to a reference sequence numbered according to the amino acid sequence of SEQ ID NO: 138, 981, or 982. In some embodiments, [N1] replaces positions 453-455 (e.g., G453, 5454, and G455), relative to a reference sequence numbered according to SEQ ID NO: 138. In some embodiments, the AAV capsid variant comprises H at position 454 and D at position 455, numbered according to SEQ ID NO: 138 or 982. In some embodiments, the AAV capsid variant comprises S at position 454 and G at position 455, numbered according to SEQ ID NO: 138 or 981. In some embodiments, an insert of 8 amino acids replaces the SG at positions 454-455, numbered according to SEQ ID NO: 138. In some embodiments, an insert of 6 amino acids is present immediately subsequent to position 455, numbered according to SEQ ID NO: 138, 981, or 982.

In yet another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises an amino acid sequence having the following formula: [N1]-[N2]-[N3], wherein: (i) [N1] comprises positions X1, X2, and X3, wherein position X2 is S and position X3 is G; (ii) [N2] comprises the amino acid sequence SPH; and (iii) [N3] comprises positions X4, X5, and X6, wherein position X5 is K (SEQ ID NO: 6612). In some embodiments, [N1]-[N2]-[N3] is present immediately subsequent to position 452 and replaces positions 453-455, numbered according to SEQ ID NO: 138 or 982. In some embodiments, [N1]-[N2]-[N3] is or comprises GSGSPHSKA (SEQ ID NO: 6613).

In another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises an amino acid sequence having the following formula: [N1]-[N2]-[N3], wherein: (i) [N1] comprises positions X1, X2, and X3, wherein position X2 is an amino acid other than S and position X3 is an amino acid other than G; (ii) [N2] comprises the amino acid sequence SPH; and (iii) [N3] comprises positions X4, X5, and X6, wherein position X4 is K (SEQ ID NO: 6614). In some embodiments, [N1]-[N2]-[N3] is present immediately subsequent to position 452 and replaces positions 453-455, numbered according to SEQ ID NO: 138 or 982. In some embodiments, [N1]-[N2]-[N3] is or comprises GHDSPHKSG (SEQ ID NO: 6615).

In another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises (a) the amino acid sequence of any of the sequences provided in Tables 1A, 2A, 2B, 15, or 16; (b) an amino acid sequence comprising at least 3, 4, 5, 6, 7, 8, 9, 10, 11, 12, 13, 14, 15, 16, or 17, consecutive amino acids from any one of the sequences provided in Tables 1A, 2A, 2B, 9-11, 15, or 16; (c) an amino acid sequence comprising at least one, two, or three, but no more than four different amino acids, relative to any one of the sequences provided in Tables 1A, 2A, 2B, 15, or 16; or (d) an amino acid sequence comprising at least one, two, or three but no more than four modifications, e.g., substitutions (e.g., conservative substitutions), relative to the amino acid sequence of any one of the sequences provided in Tables 1A, 2A, 2B, 9-11, 15, or 16. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138.

In yet another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises (a) the amino acid sequence of any of SEQ ID NOs: 945-980 or 985-986; (b) an amino acid sequence comprising at least 3, 4, or 5 consecutive amino acids from any one of SEQ ID NOs: 945-980 or 985-986; (c) an amino acid sequence comprising at least one, two, or three but no more than four different amino acids, relative to the amino acid sequence of any one of SEQ ID NOs: 945-980 or 985-986; (d) an amino acid sequence comprising at least one, two, or three but no more than four modifications, e.g., substitutions (e.g., conservative substitutions), relative to the amino acid sequence of any one of SEQ ID NOs: 945-980 or 985-986. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138.

In yet another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises (a) the amino acid sequence of any of SEQ ID NOs: 2, 200, 201, 941, 943, 204, 208, 404, or 903-909; (b) an amino acid sequence comprising at least 3, 4, 5, 6, 7, 8, 9, 10, 11, 12, or 13 consecutive amino acids from any one of SEQ ID NOs: 2, 200, 201, 941, 943, 204, 208, 404, or 903-909; (c) an amino acid sequence comprising at least one, two, or three, but no more than four different amino acids, relative to the amino acid sequence of any one of SEQ ID NOs: 2, 200, 201, 941, 943, 204, 208, 404, or 903-909; or (d) an amino acid sequence comprising at least one, two, or three but no more than four modifications, e.g., substitutions (e.g., conservative substitutions), relative to the amino acid sequence of any one of SEQ ID NOs: 2, 200, 201, 941, 943, 204, 208, 404, or 903-909. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138.

In yet another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises the amino acid sequence of SPH, wherein the amino acid sequence is present immediately subsequent to position 455, numbered according to the amino acid sequence of any one of SEQ ID NOs: 36-59, 138, 981, or 982.

In yet another aspect, present disclosure provides an AAV particle comprising an AAV capsid variant and a nucleic acid comprising a transgene encoding an antibody molecule which binds to HER2/neu (e.g., human HER2/neu), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 138.

In yet another aspect, present disclosure provides an AAV particle comprising an AAV capsid variant and a nucleic acid comprising a transgene encoding an antibody molecule which binds to HER2/neu (e.g., human HER2/neu), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 981.

In yet another aspect, present disclosure provides an AAV particle comprising an AAV capsid variant and a nucleic acid comprising a transgene encoding an antibody molecule which binds to HER2/neu (e.g., human HER2/neu), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 138.

In yet another aspect, present disclosure provides an AAV particle comprising an AAV capsid variant and a nucleic acid comprising a transgene encoding an antibody molecule which binds to HER2/neu (e.g., human HER2/neu), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 982.

In yet another aspect, present disclosure provides an AAV particle comprising an AAV capsid variant and a nucleic acid comprising a transgene encoding an antibody molecule which binds to HER2/neu (e.g., human HER2/neu), wherein the AAV capsid variant comprises an amino acid sequence comprising at least 3, 4, 5, or 6 consecutive amino acids from the amino acid sequence of SPHSKA (SEQ ID NO: 941), wherein: (i) the 3 consecutive amino acids comprise SPH; (ii) the 4 consecutive amino acids comprise SPHS (SEQ ID NO: 6616); (iii) the 5 consecutive amino acids comprise SPHSK (SEQ ID NO: 6617); or (iv) the 6 consecutive amino acids comprise SPHSKA (SEQ ID NO: 941); wherein the AAV capsid variant comprises: (a) a VP1 protein comprising the amino acid sequence of SEQ ID NO: 138 or SEQ ID NO: 981; (b) a VP2 protein comprising the amino acid sequence of positions 138-736 of SEQ ID NO: 138 or positions 138-742 of SEQ ID NO: 981; (c) a VP3 protein comprising the amino acid sequence of positions 203-736 of SEQ ID NO: 138 or positions 203-742 of SEQ ID NO: 981; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 455, numbered according to SEQ ID NO: 138 or 981.

In yet another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises one or two, but no more than three substitutions relative to the amino acid sequence of SPHSKA (SEQ ID NO: 941), wherein the AAV capsid variant comprises: (a) a VP1 protein comprising the amino acid sequence of SEQ ID NO: 138 or SEQ ID NO: 981; (b) a VP2 protein comprising the amino acid sequence of positions 138-736 of SEQ ID NO: 138 or positions 138-742 of SEQ ID NO: 981; (c) a VP3 protein comprising the amino acid sequence of positions 203-736 of SEQ ID NO: 138 or positions 203-742 of SEQ ID NO: 981; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 455, numbered according to SEQ ID NO: 138 or 981.

In another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises at least 3, 4, 5, or 6 consecutive amino acids from the amino acid sequence of HDSPHK (SEQ ID NO: 2), wherein: (i) the 3 consecutive amino acids comprise HDS; (ii) the 4 consecutive amino acids comprise HDSP (SEQ ID NO: 6618); (iii) the 5 consecutive amino acids comprise HDSPH (SEQ ID NO: 6619); and/or (iv) the 6 consecutive amino acids comprise HDSPHK (SEQ ID NO: 2); wherein the AAV capsid variant comprises: (a) a VP1 protein comprising the amino acid sequence of SEQ ID NO: 138 or SEQ ID NO: 982; (b) a VP2 protein comprising the amino acid sequence of positions 138-736 of SEQ ID NO: 138 or positions 138-742 of SEQ ID NO: 982; (c) a VP3 protein comprising the amino acid sequence of positions 203-736 of SEQ ID NO: 138 or positions 203-742 of SEQ ID NO: 982; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 453, numbered according to SEQ ID NO: 138 or 982.

In another aspect, the present disclosure provides an AAV particle comprising an AAV capsid variant and a nucleic acid comprising a transgene encoding an antibody molecule which binds to HER2/neu (e.g., human HER2/neu), wherein the AAV capsid variant comprises one or two, but no more than three substitutions relative to the amino acid sequence of HDSPHK (SEQ ID NO: 2), wherein the AAV capsid variant comprises: (a) a VP1 protein comprising the amino acid sequence of SEQ ID NO: 138 or SEQ ID NO: 982; (b) a VP2 protein comprising the amino acid sequence of positions 138-736 of SEQ ID NO: 138 or positions 138-742 of SEQ ID NO: 982; (c) a VP3 protein comprising the amino acid sequence of positions 203-736 of SEQ ID NO: 138 or positions 203-742 of SEQ ID NO: 982; or (d) an amino acid sequence with at least 90% (e.g., at least about 95, 96, 97, 98, or 99%) sequence identity to any of the amino acid sequences in (a)-(c). In some embodiments, the amino acid sequence is present immediately subsequent to positions 453, numbered according to SEQ ID NO: 138 or 982.

Accordingly, in one aspect, the present disclosure provides an isolated, e.g., recombinant nucleic acid comprising a transgene encoding an antibody molecule that binds to HER2, which comprises a heavy chain variable region (VH) encoded by a nucleotide sequence comprising a nucleotide sequence with at least 80% (e.g., at least about 85, 90, 95, 96, 97, 98, or 99%) sequence identity to the nucleotide sequence of SEQ ID NO: 5109 and/or a light chain variable region (VL) encoded by a nucleotide sequence comprising a nucleotide sequence with at least 80% (e.g., at least about 85, 90, 95, 96, 97, 98, or 99%) sequence identity to the nucleotide sequence of SEQ ID NO: 5113.

Accordingly, in one aspect, the present disclosure provides an isolated, e.g., recombinant nucleic acid comprising a transgene encoding an antibody molecule that binds to HER2, which comprises a heavy chain variable region (VH) encoded by a nucleotide sequence comprising a nucleotide sequence with at least 80% (e.g., at least about 85, 90, 95, 96, 97, 98, or 99%) sequence identity to the nucleotide sequence of SEQ ID NO: 5269 and/or a light chain variable region (VL) encoded by a nucleotide sequence comprising a nucleotide sequence with at least 80% (e.g., at least about 85, 90, 95, 96, 97, 98, or 99%) sequence identity to the nucleotide sequence of SEQ ID NO: 5273.

In another aspect, the present disclosure provides a genetic element comprising a promoter operably linked to a transgene encoding an antibody molecule that binds to HER2 (e.g., an anti-HER2 antibody molecule described herein), wherein the transgene is encoded by an isolated nucleic acid molecule described herein. In some embodiments, the genetic element further comprises an internal terminal repeat (ITR) sequence (e.g., an ITR region described herein), an enhancer (e.g., an enhancer described herein), an intron region (e.g., an intron region described herein) and/or an exon region (e.g., an exon region described herein), and/or a poly A signal region (e.g., a poly A signal sequence described herein). In some embodiments, the genetic element comprises the nucleotide sequence of any one of SEQ ID NOs: 5163, 5170, 5164, 5165, 5166, 5185, 5186, 5167, 5168, 5187, 5188, 5619, 5189, 5190, 5343, 5374, 5375, 6500, 6501, 6502, 6503, 6504, 6505, 6506, 6507, 6508, or 6509, or a sequence with at least 95% sequence identity thereto.

In yet another aspect, the present disclosure provides an isolated, e.g., recombinant, genetic element comprising a nucleic acid positioned between two inverted terminal repeats (ITRs), wherein the nucleic acid comprising a transgene encoding a multispecific, e.g., bispecific, antibody molecule comprising at least two antigen binding domains for two different domains of HER2. In some embodiments, the first antigen binding domain binds to domain I of HER2, and the second antigen binding domain binds to domain IV of HER2.

In yet another aspect, the present disclosure provides an isolated, e.g., recombinant, adeno-associated viral (AAV) vector comprising a transgene encoding an antibody molecule that binds to HER2 described herein. In some embodiments, the AAV vector comprises a genetic element comprising a promoter operably linked to a transgene encoding an antibody molecule that binds to HER2 described herein.

In yet another aspect, the present disclosure provides an isolated, e.g., recombinant, AAV particle comprising an AAV capsid polypeptide, e.g., an AAV capsid variant, and a nucleic acid comprising a transgene encoding an antibody molecule that binds to HER2 described herein. In some embodiments, the AAV particle comprises a genetic element comprising a promoter operably linked to a transgene encoding an antibody molecule that binds to HER2 described herein. In some embodiments, the AAV particle comprises an AAV vector described herein. In some embodiments, the AAV capsid polypeptide, comprises a VOY101 capsid polypeptide, a VOY9P39 capsid polypeptide, a VOY9P33 capsid protein, a AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAV9 K449R capsid polypeptide, an AAVrh10 capsid polypeptide, or a functional variant thereof.

In yet another aspect, the present disclosure provides a method of making a genetic element. The method comprising providing a nucleic acid encoding a genetic element described herein and a backbone region suitable for replication of the genetic element in a cell, e.g., a bacterial cell (e.g., wherein the backbone region comprises one or both of a bacterial origin of replication and a selectable marker), and excising the genetic element from the backbone region, e.g., by cleaving the nucleic acid molecule at upstream and downstream of the genetic element.

In yet another aspect, the present disclosure provides a method of making an isolated, e.g., recombinant AAV particle. The method comprising providing a host cell comprising a genetic element described herein and incubating the host cell under conditions suitable to enclose the genetic element in the AAV particle, e.g., a VOY101 capsid protein, thereby making the isolated AAV particle.

In yet another aspect, the present disclosure provides method of delivering an exogenous antibody molecule that binds to HER2 (e.g., an anti-HER2 antibody molecule described herein), to a subject. The method comprising administering an effective amount of an AAV particle or a plurality of AAV particles, described herein, said AAV particle comprising an AAV vector and/or genetic element described herein.

In yet another aspect, the present disclosure provides a method of treating a subject having or being diagnosed as having disease and/or a disorder associated with over-expression of HER2. The method comprising administering to the subject an effective amount of an AAV particle or a plurality of AAV particles, described herein, comprising an AAV vector and/or genetic element described herein. In some embodiments, the disease and/or disorder associated with over-expression of HER2 includes tumors, cancers, and neoplastic tissue, along with pre-malignant and non-neoplastic or non-malignant hyperproliferative disorders.

Those skilled in the art will recognize or be able to ascertain using no more than routine experimentation, many equivalents to the specific embodiments of the invention described herein. Such equivalents are intended to be encompassed by the following enumerated embodiments.

### ENUMERATED EMBODIMENTS

1. An AAV particle comprising an AAV capsid variant and a nucleic acid comprising a transgene encoding an antibody molecule which binds to HER2/neu (e.g., human HER2/neu), wherein the AAV capsid variant comprises an amino acid sequence having the following formula: [N1]-[N2]-[N3], wherein:
(i) optionally [N1] comprises X1, X2, and X3, wherein at least one of X1, X2, or X3 is G;
(ii) [N2] comprises the amino acid sequence of SPH;
(ii) [N3] comprises X4, X5, and X6, wherein at least one of X4, X5, or X6 is a basic amino acid, e.g., a K or R.
   2. The AAV particle of embodiment 1, wherein X4, X5, or both of [N3] is a K.
   3. The AAV particle of embodiment 1 or 2, wherein X4, X5, or X6 of [N3] is an R.
   4. The AAV particle of any one of embodiments 1-3, wherein:
(a) position X4 of [N3] is: K, S, A, V, T, G, F, W, V, N, or R;
(b) position X5 of [N3] is: S, K, T, F, I, L, Y, H, M, or R; and/or
(c) position X6 of [N3] is: G, A, R, M, I, N, T, Y, D, P, V, L, E, W, N, Q, K, or S;
optionally wherein the AAV capsid variant comprises an amino acid modification, e.g., a conservative substitution, of any of the aforesaid amino acids in (a)-(c).
   5. The AAV particle of any one of embodiments 1-4, wherein [N3] comprises SK, KA, KS, AR, RM, VK, AS, SR, VK, KR, KK, KN, VR, RS, RK, KT, TS, KF, FG, KI, IG, KL, LG, TT, TY, KY, YG, KD, KP, TR, RG, VR, GA, SL, SS, FL, WK, SA, RA, LR, KW, RR, GK, TK, NK, AK, KV, KG, KH, KM, TG, SE, SV, SW, SN, HG, SQ, LW, MG, MA, or SG.
   6. The AAV particle of any one of embodiments 1-5, wherein [N3] is or comprises SKA, KSG, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG.
   7. The AAV particle of any one of embodiments 1-6, wherein [N2]-[N3] comprises SPHSK (SEQ ID NO: 6617), SPHKS (SEQ ID NO: 6620), SPHAR (SEQ ID NO: 6621), SPHVK (SEQ ID NO: 6622), SPHAS (SEQ ID NO: 6623), SPHKK (SEQ ID NO: 6624), SPHVR (SEQ ID NO: 6625), SPHRK (SEQ ID NO: 6626), SPHKT (SEQ ID NO: 6627), SPHKF (SEQ ID NO: 6628), SPHKI (SEQ ID NO: 6629), SPHKL (SEQ ID NO: 6630), SPHKY (SEQ ID NO: 6631), SPHTR (SEQ ID NO: 6632), SPHKR (SEQ ID NO: 6633), SPHGA (SEQ ID NO: 6634), SPHSR (SEQ ID NO: 6635), SPHSL (SEQ ID NO: 6636), SPHSS (SEQ ID NO: 6637), SPHFL (SEQ ID NO: 6638), SPHWK (SEQ ID NO: 6639), SPHGK (SEQ ID NO: 6640), SPHTK (SEQ ID NO: 6641), SPHNK (SEQ ID NO: 6642), SPHAK (SEQ ID NO: 6643), SPHKH (SEQ ID NO: 6644), SPHKM (SEQ ID NO: 6645), or SPHRS (SEQ ID NO: 6646).
   8. The AAV particle of any one of embodiments 1-7, wherein [N2]-[N3] is or comprises:

(i)
(SEQ ID NO: 941)
SPHSKA,
(SEQ ID NO: 946)
SPHKSG,
(SEQ ID NO: 947)
SPHARM,
(SEQ ID NO: 948)
SPHVKS,
(SEQ ID NO: 949)
SPHASR,
(SEQ ID NO: 950)
SPHVKI,
(SEQ ID NO: 954)
SPHKKN,
(SEQ ID NO: 955)
SPHVRM,
(SEQ ID NO: 956)
SPHRKA,
(SEQ ID NO: 957)
SPHKFG,
(SEQ ID NO: 958)
SPHKIG,
(SEQ ID NO: 959)
SPHKLG,
(SEQ ID NO: 963)
SPHKTS,
(SEQ ID NO: 964)
SPHKTT,
(SEQ ID NO: 965)
SPHKTY,
(SEQ ID NO: 966)
SPHKYG,
(SEQ ID NO: 967)
SPHSKD,
(SEQ ID NO: 968)
SPHSKP,
(SEQ ID NO: 972)
SPHTRG,
(SEQ ID NO: 973)
SPHVRG,
(SEQ ID NO: 974)
SPHKRG,
(SEQ ID NO: 975)
SPHGAR,
(SEQ ID NO: 977)
SPHKSA,
(SEQ ID NO: 951)
SPHKSR,
(SEQ ID NO: 960)
SPHSKL,
(SEQ ID NO: 969)
SPHSRA,
(SEQ ID NO: 978)
SPHSKR,
(SEQ ID NO: 952)
SPHSLR,
(SEQ ID NO: 961)
SPHSRG,
(SEQ ID NO: 970)
SPHSSR,
(SEQ ID NO: 979)
SPHFLR,
(SEQ ID NO: 953)
SPHSKW,
(SEQ ID NO: 962)
SPHSKS,
(SEQ ID NO: 971)
SPHWKA,
(SEQ ID NO: 980)
SPHVRR,
(SEQ ID NO: 6647)
SPHSKT,
(SEQ ID NO: 6648)
SPHSKG,
(SEQ ID NO: 6649)
SPHGKA,
(SEQ ID NO: 6650)
SPHNKA,
(SEQ ID NO: 6651)
SPHSKN,
(SEQ ID NO: 6652)
SPHAKA,
(SEQ ID NO: 6653)
SPHSKV,
(SEQ ID NO: 6654)
SPHKTG,
(SEQ ID NO: 6655)
SPHTKA,
(SEQ ID NO: 6656)
SPHKSL,
(SEQ ID NO: 6657)
SPHKSE,
(SEQ ID NO: 6658)
SPHKSV,
(SEQ ID NO: 6659)
SPHKSW,
(SEQ ID NO: 6660)
SPHKSN,
(SEQ ID NO: 6661)
SPHKHG,
(SEQ ID NO: 6662)
SPHKSQ,
(SEQ ID NO: 6663)
SPHKSK,
(SEQ ID NO: 6664)
SPHKLW,
(SEQ ID NO: 6665)
SPHWKG,
(SEQ ID NO: 6666)
SPHKMG,
(SEQ ID NO: 6667)
SPHKMA,
or
(SEQ ID NO: 976)
SPHRSG;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   9. The AAV particle of any one of embodiments 1-8, wherein the AAV capsid variant comprises an amino acid other than G at position 453 (e.g., V, R, D, E, M, T, I, S, A, N, L, K, H, P, W, or C), an amino acid other than S at position 454 (V, L, N, D, H, R, P, G, T, I, A, E, Y, M, or Q), and/or a G at position 455 (e.g., C, L, D, E, Y, H, V, A, N, P, or S), numbered according to any one of SEQ ID NOs: 36-59, 138, 981 or 982.
   10. The AAV particle of any one of embodiments 1-8, wherein the AAV capsid variant comprises the amino acid G at position 453, the amino acid S at position 454, and the amino acid G at position 455, numbered according to SEQ ID NO: 138 or 981.
   11. The AAV particle of any one of embodiments 1-9, wherein the AAV capsid variant comprises the amino acid G at position 453, the amino acid H at position 454, and the amino acid D at position 455, numbered according to SEQ ID NO: 138 or 982.
   12. The AAV particle of any one of embodiments 1-11, wherein [N1] comprises X1. X2, and X3, wherein at least one of X1, X2, or X3 is G.
   13. The AAV particle of any one of embodiments 1-12, wherein:
(a) position X1 of [N1] is: G, V, R, D, E, M, T, I, S, A, N, L, K, H, P, W, or C;
(b) position X2 of [N1] is: S, V, L, N, D, H, R, P, G, T, I, A, E, Y, M, or Q; and/or
(c) position X3 of [N1] is: G, C, L, D, E, Y, H, V, A, N, P, or S;
optionally wherein the AAV capsid variant comprises an amino acid modification, e.g., a conservative substitution, of any of the aforesaid amino acids in (a)-(c).
   14. The AAV particle of any one of embodiments 1-13, wherein [N1] comprises GS, SG, GH, HD, GQ, QD, VS, CS, GR, RG, QS, SH, MS, RN, TS, IS, GP, ES, SS, GN, AS, NS, LS, GG, KS, GT, PS, RS, GI, WS, DS, ID, GL, DA, DG, ME, EN, KN, KE, AI, NG, PG, TG, SV, IG, LG, AG, EG, SA, YD, HE, HG, RD, ND, PD, MG, QV, DD, HN, HP, GY, GM, GD, or HS.
   15. The AAV particle of any one of embodiments 1-14, wherein [N1] is or comprises GSG, GHD, GQD, VSG, CSG, GRG, CSH, GQS, GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AITG, GYD, GHG, GRD, GND, GPD, GMG, GQV, GHN, GHP, or GHS.
   16. The AAV particle of any one of embodiments 1-15, wherein [N1]-[N2] comprises

(i)
(SEQ ID NO: 6668)
SGSPH,
(SEQ ID NO: 6619)
HDSPH,
(SEQ ID NO: 6669)
QDSPH,
(SEQ ID NO: 6670)
RGSPH,
(SEQ ID NO: 6671)
SHSPH,
(SEQ ID NO: 6672)
QSSPH,
(SEQ ID NO: 6673)
DDSPH,
(SEQ ID NO: 6674)
HESPH,
(SEQ ID NO: 6675)
NYSPH,
(SEQ ID NO: 6676)
VGSPH,
(SEQ ID NO: 6677)
SCSPH,
(SEQ ID NO: 6678)
LLSPH,
(SEQ ID NO: 6679)
NGSPH,
(SEQ ID NO: 6680)
PGSPH,
(SEQ ID NO: 6681)
GGSPH,
(SEQ ID NO: 6682)
TGSPH,
(SEQ ID NO: 6683)
SVSPH,
(SEQ ID NO: 6684)
IGSPH,
(SEQ ID NO: 6685)
DGSPH,
(SEQ ID NO: 6686)
LGSPH,
(SEQ ID NO: 6687)
AGSPH,
(SEQ ID NO: 6688)
EGSPH,
(SEQ ID NO: 6689)
SASPH,
(SEQ ID NO: 6690)
YDSPH,
(SEQ ID NO: 6691)
HGSPH,
(SEQ ID NO: 6692)
RDSPH,
(SEQ ID NO: 6693)
NDSPH,
(SEQ ID NO: 6694)
PDSPH,
(SEQ ID NO: 6695)
MGSPH,
(SEQ ID NO: 6696)
QVSPH,
(SEQ ID NO: 6697)
HNSPH,
(SEQ ID NO: 6698)
HPSPH,
or
(SEQ ID NO: 6699)
HSSPH;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   17. The AAV article of an one of embodiments 1-16, wherein N1-N2 is or comprises:

(i)
(SEQ ID NO: 6700)
GSGSPH,
(SEQ ID NO: 6701)
GHDSPH,
(SEQ ID NO: 6702)
GQDSPH,
(SEQ ID NO: 6703)
VSGSPH,
(SEQ ID NO: 6704)
CSGSPH,
(SEQ ID NO: 6705)
GRGSPH,
(SEQ ID NO: 6706)
CSHSPH,
(SEQ ID NO: 6707)
GQSSPH,
(SEQ ID NO: 6708)
GSHSPH,
(SEQ ID NO: 6709)
GDDSPH,
(SEQ ID NO: 6710)
GHESPH,
(SEQ ID NO: 6711)
GNYSPH,
(SEQ ID NO: 6712)
RVGSPH,
(SEQ ID NO: 6713)
GSCSPH,
(SEQ ID NO: 6714)
GLLSPH,
(SEQ ID NO: 6715)
MSGSPH,
(SEQ ID NO: 6716)
RNGSPH,
(SEQ ID NO: 6717)
TSGSPH,
(SEQ ID NO: 6718)
ISGSPH,
(SEQ ID NO: 6719)
GPGSPH,
(SEQ ID NO: 6720)
ESGSPH,
(SEQ ID NO: 6721)
SSGSPH,
(SEQ ID NO: 6722)
GNGSPH,
(SEQ ID NO: 6723)
ASGSPH,
(SEQ ID NO: 6724)
NSGSPH,
(SEQ ID NO: 6725)
LSGSPH,
(SEQ ID NO: 6726)
GGGSPH,
(SEQ ID NO: 6727)
KSGSPH,
(SEQ ID NO: 6728)
HSGSPH,
(SEQ ID NO: 6729)
GTGSPH,
(SEQ ID NO: 6730)
PSGSPH,
(SEQ ID NO: 6731)
GSVSPH,
(SEQ ID NO: 6732)
RSGSPH,
(SEQ ID NO: 6733)
GIGSPH,
(SEQ ID NO: 6734)
WSGSPH,
(SEQ ID NO: 6735)
DSGSPH,
(SEQ ID NO: 6736)
IDGSPH,
(SEQ ID NO: 6737)
GLGSPH,
(SEQ ID NO: 6738)
DAGSPH,
(SEQ ID NO: 6739)
DGGSPH,
(SEQ ID NO: 6740)
MEGSPH,
(SEQ ID NO: 6741)
ENGSPH,
(SEQ ID NO: 6742)
GSASPH,
(SEQ ID NO: 6743)
KNGSPH,
(SEQ ID NO: 6744)
KEGSPH,
(SEQ ID NO: 6745)
AIGSPH,
(SEQ ID NO: 6746)
GYDSPH,
(SEQ ID NO: 6747)
GHGSPH,
(SEQ ID NO: 6748)
GRDSPH,
(SEQ ID NO: 6749)
GNDSPH,
(SEQ ID NO: 6750)
GPDSPH,
(SEQ ID NO: 6751)
GMGSPH,
(SEQ ID NO: 6752)
GQVSPH,
(SEQ ID NO: 6753)
GHNSPH,
(SEQ ID NO: 6754)
GHPSPH,
or
(SEQ ID NO: 6755)
GHSSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   18. The AAV particle of any one of embodiments 1-17, wherein [N1]-[N2]-[N3] comprises:

(i)
(SEQ ID NO: 6756)
SGSPHSK,
(SEQ ID NO: 6757)
HDSPHKS,
(SEQ ID NO: 6758)
SGSPHAR,
(SEQ ID NO: 6759)
SGSPHVK,
(SEQ ID NO: 6760)
QDSPHKS,
(SEQ ID NO: 6761)
SGSPHKK,
(SEQ ID NO: 6762)
SGSPHVR,
(SEQ ID NO: 6763)
SGSPHAS,
(SEQ ID NO: 6764)
SGSPHRK,
(SEQ ID NO: 6765)
SGSPHKT,
(SEQ ID NO: 6766)
SHSPHKS,
(SEQ ID NO: 6767)
QSSPHRS,
(SEQ ID NO: 6768)
RGSPHAS,
(SEQ ID NO: 6769)
RGSPHSK,
(SEQ ID NO: 6770)
SGSPHKF,
(SEQ ID NO: 6771)
SGSPHKI,
(SEQ ID NO: 6772)
SGSPHKL,
(SEQ ID NO: 6773)
SGSPHKY,
(SEQ ID NO: 6774)
SGSPHTR,
(SEQ ID NO: 6775)
SHSPHKR,
(SEQ ID NO: 6776)
SGSPHGA,
(SEQ ID NO: 6777)
HDSPHKR,
(SEQ ID NO: 6778)
DDSPHKS,
(SEQ ID NO: 6779)
HESPHKS,
(SEQ ID NO: 6780)
NYSPHKI,
(SEQ ID NO: 6781)
SGSPHSR,
(SEQ ID NO: 6782)
SGSPHSL,
(SEQ ID NO: 6783)
SGSPHSS,
(SEQ ID NO: 6784)
VGSPHSK,
(SEQ ID NO: 6785)
SCSPHRK,
(SEQ ID NO: 6786)
SGSPHFL,
(SEQ ID NO: 6787)
LLSPHWK,
(SEQ ID NO: 6788)
NGSPHSK,
(SEQ ID NO: 6789)
PGSPHSK,
(SEQ ID NO: 6790)
GGSPHSK,
(SEQ ID NO: 6791)
TGSPHSK,
(SEQ ID NO: 6792)
SVSPHGK,
(SEQ ID NO: 6793)
SGSPHTK,
(SEQ ID NO: 6794)
IGSPHSK,
(SEQ ID NO: 6795)
DGSPHSK,
(SEQ ID NO: 6796)
SGSPHNK,
(SEQ ID NO: 6797)
LGSPHSK,
(SEQ ID NO: 6798)
AGSPHSK,
(SEQ ID NO: 6799)
EGSPHSK,
(SEQ ID NO: 6800)
SASPHSK,
(SEQ ID NO: 6801)
SGSPHAK,
(SEQ ID NO: 6802)
HDSPHKI,
(SEQ ID NO: 6803)
YDSPHKS,
(SEQ ID NO: 6804)
HDSPHKT,
(SEQ ID NO: 6805)
RGSPHKR,
(SEQ ID NO: 6806)
HGSPHSK,
(SEQ ID NO: 6807)
RDSPHKS,
(SEQ ID NO: 6808)
NDSPHKS,
(SEQ ID NO: 6809)
QDSPHKI,
(SEQ ID NO: 6810)
PDSPHKI,
(SEQ ID NO: 6811)
PDSPHKS,
(SEQ ID NO: 6812)
MGSPHSK,
(SEQ ID NO: 6813)
HDSPHKH,
(SEQ ID NO: 6814)
QVSPHKS,
(SEQ ID NO: 6815)
HNSPHKS,
(SEQ ID NO: 6816)
NGSPHKR,
(SEQ ID NO: 6817)
HDSPHKY,
(SEQ ID NO: 6818)
NDSPHKI,
(SEQ ID NO: 6819)
HDSPHKL,
(SEQ ID NO: 6820)
HPSPHWK,
(SEQ ID NO: 6821)
HDSPHKM,
or
(SEQ ID NO: 6822)
HSSPHRS;

(ii) an amino acid sequence comprising any portion of an amino acid sequence in (i), e.g., any 2, 3, 4, 5, or 6 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   19. The AAV particle of any one of embodiments 1-18, wherein [N1]-[N2]-[N3] is or comprises:

(i)
(SEQ ID NO: 6613)
GSGSPHSKA,
(SEQ ID NO: 6615)
GHDSPHKSG,
(SEQ ID NO: 6823)
GSGSPHARM,
(SEQ ID NO: 6824)
GSGSPHVKS,
(SEQ ID NO: 6825)
GQDSPHKSG,
(SEQ ID NO: 6826)
GSGSPHASR,
(SEQ ID NO: 6827)
GSGSPHVKI,
(SEQ ID NO: 6828)
GSGSPHKKN,
(SEQ ID NO: 6829)
GSGSPHVRM,
(SEQ ID NO: 6830)
VSGSPHSKA,
(SEQ ID NO: 6831)
CSGSPHSKA,
(SEQ ID NO: 6832)
GSGSPHRKA,
(SEQ ID NO: 6833)
CSGSPHKTS,
(SEQ ID NO: 6834)
CSHSPHKSG,
(SEQ ID NO: 6835)
GQSSPHRSG,
(SEQ ID NO: 6836)
GRGSPHASR,
(SEQ ID NO: 6837)
GRGSPHSKA,
(SEQ ID NO: 6838)
GSGSPHKFG,
(SEQ ID NO: 6839)
GSGSPHKIG,
(SEQ ID NO: 6840)
GSGSPHKLG,
(SEQ ID NO: 6841)
GSGSPHKTS,
(SEQ ID NO: 6842)
GSGSPHKTT,
(SEQ ID NO: 6843)
GSGSPHKTY,
(SEQ ID NO: 6844)
GSGSPHKYG,
(SEQ ID NO: 6845)
GSGSPHSKD,
(SEQ ID NO: 6846)
GSGSPHSKP,
(SEQ ID NO: 6847)
GSGSPHTRG,
(SEQ ID NO: 6848)
GSGSPHVRG,
(SEQ ID NO: 6849)
GSHSPHKRG,
(SEQ ID NO: 6850)
GSHSPHKSG,
(SEQ ID NO: 6851)
VSGSPHASR,
(SEQ ID NO: 6852)
VSGSPHGAR,
(SEQ ID NO: 6853)
VSGSPHKFG,
(SEQ ID NO: 6854)
GHDSPHKRG,
(SEQ ID NO: 6855)
GDDSPHKSG,
(SEQ ID NO: 6856)
GHESPHKSA,
(SEQ ID NO: 6857)
GHDSPHKSA,
(SEQ ID NO: 6858)
GNYSPHKIG,
(SEQ ID NO: 6859)
GHDSPHKSR,
(SEQ ID NO: 6860)
GSGSPHSKL,
(SEQ ID NO: 6861)
GSGSPHSRA,
(SEQ ID NO: 6862)
GSGSPHSKR,
(SEQ ID NO: 6863)
GSGSPHSLR,
(SEQ ID NO: 6864)
GSGSPHSRG,
(SEQ ID NO: 6865)
GSGSPHSSR,
(SEQ ID NO: 6866)
RVGSPHSKA,
(SEQ ID NO: 6867)
GSCSPHRKA,
(SEQ ID NO: 6868)
GSGSPHFLR,
(SEQ ID NO: 6869)
GSGSPHSKW,
(SEQ ID NO: 6870)
GSGSPHSKS,
(SEQ ID NO: 6871)
GLLSPHWKA,
(SEQ ID NO: 6872)
GSGSPHVRR,
(SEQ ID NO: 6873)
GSGSPHSKV,
(SEQ ID NO: 6874)
MSGSPHSKA,
(SEQ ID NO: 6875)
RNGSPHSKA,
(SEQ ID NO: 6876)
TSGSPHSKA,
(SEQ ID NO: 6877)
ISGSPHSKA,
(SEQ ID NO: 6878)
GPGSPHSKA,
(SEQ ID NO: 6879)
GSGSPHSKT,
(SEQ ID NO: 6880)
ESGSPHSKA,
(SEQ ID NO: 6881)
SSGSPHSKA,
(SEQ ID NO: 6882)
GNGSPHSKA,
(SEQ ID NO: 6883)
ASGSPHSKA,
(SEQ ID NO: 6884)
NSGSPHSKA,
(SEQ ID NO: 6885)
LSGSPHSKA,
(SEQ ID NO: 6886)
GGGSPHSKA,
(SEQ ID NO: 6887)
KSGSPHSKA,
(SEQ ID NO: 6888)
GGGSPHSKS,
(SEQ ID NO: 6889)
GSGSPHSKG,
(SEQ ID NO: 6890)
HSGSPHSKA,
(SEQ ID NO: 6891)
GTGSPHSKA,
(SEQ ID NO: 6892)
PSGSPHSKA,
(SEQ ID NO: 6893)
GSVSPHGKA,
(SEQ ID NO: 6894)
RSGSPHSKA,
(SEQ ID NO: 6895)
GSGSPHTKA,
(SEQ ID NO: 6896)
GIGSPHSKA,
(SEQ ID NO: 6897)
WSGSPHSKA,
(SEQ ID NO: 6898)
DSGSPHSKA,
(SEQ ID NO: 6899)
IDGSPHSKA,
(SEQ ID NO: 6900)
GSGSPHNKA,
(SEQ ID NO: 6901)
GLGSPHSKS,
(SEQ ID NO: 6902)
DAGSPHSKA,
(SEQ ID NO: 6903)
DGGSPHSKA,
(SEQ ID NO: 6904)
MEGSPHSKA,
(SEQ ID NO: 6905)
ENGSPHSKA,
(SEQ ID NO: 6906)
GSASPHSKA,
(SEQ ID NO: 6907)
GNGSPHSKS,
(SEQ ID NO: 6908)
KNGSPHSKA,
(SEQ ID NO: 6909)
KEGSPHSKA,
(SEQ ID NO: 6910)
AIGSPHSKA,
(SEQ ID NO: 6911)
GSGSPHSKN,
(SEQ ID NO: 6912)
GSGSPHAKA,
(SEQ ID NO: 6913)
GHDSPHKIG,
(SEQ ID NO: 6914)
GYDSPHKSG,
(SEQ ID NO: 6915)
GHESPHKSG,
(SEQ ID NO: 6916)
GHDSPHKTG,
(SEQ ID NO: 6917)
GRGSPHKRG,
(SEQ ID NO: 6825)
GQDSPHKSG,
(SEQ ID NO: 6918)
GHDSPHKSL,
(SEQ ID NO: 6919)
GHGSPHSKA,
(SEQ ID NO: 6920)
GHDSPHKSE,
(SEQ ID NO: 6830)
VSGSPHSKA,
(SEQ ID NO: 6921)
GRDSPHKSG,
(SEQ ID NO: 6922)
GNDSPHKSV,
(SEQ ID NO: 6923)
GQDSPHKIG,
(SEQ ID NO: 6924)
GHDSPHKSV,
(SEQ ID NO: 6925)
GPDSPHKIG,
(SEQ ID NO: 6926)
GPDSPHKSG,
(SEQ ID NO: 6927)
GHDSPHKSW,
(SEQ ID NO: 6928)
GHDSPHKSN,
(SEQ ID NO: 6929)
GMGSPHSKT,
(SEQ ID NO: 6930)
GHDSPHKHG,
(SEQ ID NO: 6931)
GQVSPHKSG,
(SEQ ID NO: 6932)
GDDSPHKSV,
(SEQ ID NO: 6933)
GHNSPHKSG,
(SEQ ID NO: 6934)
GNGSPHKRG,
(SEQ ID NO: 6935)
GHDSPHKYG,
(SEQ ID NO: 6936)
GHDSPHKSQ,
(SEQ ID NO: 6937)
GNDSPHKIG,
(SEQ ID NO: 6938)
GHDSPHKSK,
(SEQ ID NO: 6939)
GHDSPHKLW,
(SEQ ID NO: 6940)
GHPSPHWKG,
(SEQ ID NO: 6941)
GHDSPHKMG,
(SEQ ID NO: 6942)
GHDSPHKMA,
or
(SEQ ID NO: 6943)
GHSSPHRSG;

(ii) an amino acid sequence comprising any portion of an amino acid sequence in (i), e.g., any 2, 3, 4, 5, 6, 7, or 8 amino acids. e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   20. The AAV particle of any one of embodiments 1-19, wherein [N3] comprises SK, KA, KS, or SG.
   21. The AAV particle of any one of embodiments 1-20, wherein [N3] is or comprises SKA, KSG, or KYG.
   22. The AAV particle of any one of embodiments 1-21, wherein [N2]-[N3] comprises SPHSK (SEQ ID NO: 6617), SPHKS (SEQ ID NO: 6620), or SPHKY (SEQ ID NO: 6631).
   23. The AAV particle of any one of embodiments 1-22, wherein [N2]-[N3] is or comprises SPHSKA (SEQ ID NO: 941).
   24. The AAV particle of any one of embodiments 1-22, wherein [N2]-[N3] is or comprises SPHKSG (SEQ ID NO: 946).
   25. The AAV particle of any one of embodiments 1-22, wherein [N2]-[N3] is or comprises SPHKYG (SEQ ID NO: 966).
   26. The AAV particle of any one of embodiments 1-25, wherein [N1] comprises GS, SG, GH, or HD.
   27. The AAV particle of any one of embodiments 1-26, wherein [N1] is or comprises GSG.
   28. The AAV particle of any one of embodiments 1-26, wherein [N1] is or comprises GHD.
   29. The AAV particle of any one of embodiments 1-23 or 26-27, wherein [N1]-[N2]-[N3] comprises SGSPHSK (SEQ ID NO: 6756).
   30. The AAV particle of any one of embodiments 1-22, 24, 26, or 28, wherein [N1]-[N2]-[N3] comprises HDSPHKS (SEQ ID NO: 6757).
   31. The AAV particle of any one of embodiments 1-22 or 25-27, wherein [N1]-[N2]-[N3] comprises SGSPHKYG (SEQ ID NO: 6944).
   32. The AAV particle of any one of embodiments 1-8, 10, 12-23, 26-27, or 29, wherein [N1]-[N2]-[N3] is or comprises GSGSPHSKA (SEQ ID NO: 6613).
   33. The AAV particle of any one of embodiments 1-9, 11-22, 24, 26, 28, or 30, wherein [N1]-[N2]-[N3] is or comprises GHDSPHKSG (SEQ ID NO: 6615).
   34. The AAV particle of any one of embodiments 1-8, 10, 12-22, 25-27, or 31, wherein [N1]-[N2]-[N3] is or comprises GSGSPHKYG (SEQ ID NO: 6844).
   35. The AAV particle of any one of embodiments 1-34, wherein [N1]-[N2]-[N3] replaces positions 453-455, numbered according to the amino acid sequence of SEQ ID NO: 138.
   36. The AAV particle of any one of embodiments 1-35, wherein the AAV capsid variant comprises an amino acid other than Q at position 456 (e.g., W, K, R, G, L, V, S, P, H, K, I, M, A, E, or F), an amino acid other than N at position 457 (e.g., Y, C, K, T, H, R, D, V, S, P, G, W, E, F, A, I, M, Q, or L), an amino acid other than Q at position 458 (e.g., G, K, H, R, T, L, D, A, P, I, F, V, M, W, Y, S, E, N, or Y), and/or an amino acid other than Q at position 459 (e.g., H, L, R, W, K, A, P, E, M, 1, S, G, N, Y, C, V, T, D, or V), relative to a reference sequence numbered according to SEQ ID NO: 138.
   37. The AAV capsid particle of any one of embodiments 1-36, wherein the AAV capsid variant comprises an amino acid other than Q at position 462 (e.g., W, K, R, G, L, V, S, P, H, K, I, M, A, E, or F), an amino acid other than N at position 463 (e.g., Y, C, K, T, H, R, D, V, S, P, G, W, E, F, A, I, M, Q, or L), an amino acid other than Q at position 464 (e.g., G, K, H, R, T, L, D, A, P, 1, F, V, M, W, Y, S, E, N, or Y), and/or an amino acid other than Q at position 465 (e.g., H, L, R, W, K, A, P, E, M, I, S, G, N, Y, C, V, T, D, or V), relative to a reference sequence numbered according to SEQ ID NO: 981, 982, 36, 37, 39, 40, 42-46, 48, 49, 50, 52, 53, 56, or 57.
   38. The AAV particle of any one of embodiments 1-37, wherein the AAV capsid variant comprises:
(a) the amino acid Q at position 456, the amino acid N at position 457, the amino acid Q at position 458, and/or the amino acid Q at position 459, relative to a reference sequence numbered according to SEQ ID NO: 138; or
(b) the amino acid Q at position 462, the amino acid N at position 463, the amino acid Q at position 464, and/or the amino acid Q at position 465, relative to a reference sequence numbered according to SEQ ID NO: 981, 982, 36, 37, 39, 40, 42-46, 48, 49, 50, 52, 53, 56, or 57.
   39. The AAV particle of any one of embodiments 1-38, wherein the AAV capsid variant further comprises [N4], wherein [N4] comprises X7 X8 X9 X10, and wherein:
(a) position X7 is: Q, W, K, R, G, L, V, S, P, H, K, I, M, A, E, or F;
(b) position X8 is: N, Y, C, K, T, H, R, D, V, S, P, G, W, E, F, A, I, M, Q, or L;
(c) position X9 is: Q, G, K, H, R, T, L, D, A, P, I, F, V, M, W, Y, S, E, N, or Y; and
(d) position X10 is: Q, H, L, R, W, K, A, P, E, M, I, S, G, N, Y, C, V, T, D, or V;
   optionally wherein the AAV capsid variant comprises an amino acid modification, e.g., a conservative substitution, of any of the aforesaid amino acids in (a)-(d).
   40. The AAV particle of embodiment 39, wherein:
(a) position X7 of [N4] is Q or R;
(b) position X8 of [N4] is N or R;
(c) position X9 of [N4] is Q or R; and
(d) position X10 of [N4] is Q, L, or R.
   41. The AAV particle of embodiment 39 or 40, wherein [N4] is or comprises:

(i)
(SEQ ID NO: 6945)
QNQQ,
(SEQ ID NO: 6946)
WNQQ,
(SEQ ID NO: 6947)
QYYV,
(SEQ ID NO: 6948)
RRQQ,
(SEQ ID NO: 6949)
GCGQ,
(SEQ ID NO: 6950)
LRQQ,
(SEQ ID NO: 6951)
RNQQ,
(SEQ ID NO: 6952)
VNQQ,
(SEQ ID NO: 6953)
FRLQ,
(SEQ ID NO: 6954)
FNQQ,
(SEQ ID NO: 6955)
LLQQ,
(SEQ ID NO: 6956)
SNQQ,
(SEQ ID NO: 6957)
RLQQ,
(SEQ ID NO: 6958)
LNQQ,
(SEQ ID NO: 6959)
QRKL,
(SEQ ID NO: 6960)
LRRQ,
(SEQ ID NO: 6961)
QRLR,
(SEQ ID NO: 6962)
QRRL,
(SEQ ID NO: 6963)
RRLQ,
(SEQ ID NO: 6964)
RLRQ,
(SEQ ID NO: 6965)
SKRQ,
(SEQ ID NO: 6966)
QLYR,
(SEQ ID NO: 6967)
QLTV,
(SEQ ID NO: 6968)
QNKQ,
(SEQ ID NO: 6969)
KNQQ,
(SEQ ID NO: 6970)
QKQQ,
(SEQ ID NO: 6971)
QTQQ,
(SEQ ID NO: 6972)
QNHQ,
(SEQ ID NO: 6973)
QHQQ,
(SEQ ID NO: 6974)
QNQH,
(SEQ ID NO: 6975)
QHRQ,
(SEQ ID NO: 6976)
LTQQ,
(SEQ ID NO: 6977)
QNQW,
(SEQ ID NO: 6978)
QNTH,
(SEQ ID NO: 6979)
RRRQ,
(SEQ ID NO: 6980)
QYQQ,
(SEQ ID NO: 6981)
QNDQ,
(SEQ ID NO: 6982)
QNRH,
(SEQ ID NO: 6983)
RDQQ,
(SEQ ID NO: 6984)
PNLQ,
(SEQ ID NO: 6985)
HVRQ,
(SEQ ID NO: 6986)
PNQH,
(SEQ ID NO: 6987)
HNQQ,
(SEQ ID NO: 6988)
QSQQ,
(SEQ ID NO: 6989)
QPAK,
(SEQ ID NO: 6990)
QNLA,
(SEQ ID NO: 6991)
QNQL,
(SEQ ID NO: 6992)
QGQQ,
(SEQ ID NO: 6993)
LNRQ,
(SEQ ID NO: 6994)
QNPP,
(SEQ ID NO: 6995)
QNLQ,
(SEQ ID NO: 6996)
QDQE,
(SEQ ID NO: 6997)
QDQQ,
(SEQ ID NO: 6998)
HWQQ,
(SEQ ID NO: 6999)
PNQQ,
(SEQ ID NO: 7000)
PEQQ,
(SEQ ID NO: 7001)
QRTM,
(SEQ ID NO: 7002)
LHQH,
(SEQ ID NO: 7003)
QHRI,
(SEQ ID NO: 7004)
QYIH,
(SEQ ID NO: 7005)
QKFE,
(SEQ ID NO: 7006)
QFPS,
(SEQ ID NO: 7007)
QNPL,
(SEQ ID NO: 7008)
QAIK,
(SEQ ID NO: 7009)
QNRQ,
(SEQ ID NO: 7010)
QYQH,
(SEQ ID NO: 7011)
QNPQ,
(SEQ ID NO: 7012)
QHQL,
(SEQ ID NO: 7013)
QSPP,
(SEQ ID NO: 7014)
QAKL,
(SEQ ID NO: 7015)
KSQQ,
(SEQ ID NO: 7016)
QDRP,
(SEQ ID NO: 7017)
QNLG,
(SEQ ID NO: 7018)
QAFH,
(SEQ ID NO: 7019)
QNAQ,
(SEQ ID NO: 7020)
HNQL,
(SEQ ID NO: 7021)
QKLN,
(SEQ ID NO: 7022)
QNVQ,
(SEQ ID NO: 7023)
QAQQ,
(SEQ ID NO: 7024)
QTPP,
(SEQ ID NO: 7025)
QPPA,
(SEQ ID NO: 7026)
QERP,
(SEQ ID NO: 7027)
QDLQ,
(SEQ ID NO: 7028)
QAMH,
(SEQ ID NO: 7029)
QHPS,
(SEQ ID NO: 7030)
PGLQ,
(SEQ ID NO: 7031)
QGIR,
(SEQ ID NO: 7032)
QAPA,
(SEQ ID NO: 7033)
QIPP,
(SEQ ID NO: 7034)
QTQL,
(SEQ ID NO: 7035)
QAPS,
(SEQ ID NO: 7036)
QNTY,
(SEQ ID NO: 7037)
QDKQ,
(SEQ ID NO: 7038)
QNHL,
(SEQ ID NO: 7039)
QIGM,
(SEQ ID NO: 7040)
LNKQ,
(SEQ ID NO: 7041)
PNQL,
(SEQ ID NO: 7042)
QLQQ,
(SEQ ID NO: 7043)
QRMS,
(SEQ ID NO: 7044)
QGIL,
(SEQ ID NO: 7045)
QDRQ,
(SEQ ID NO: 7046)
RDWQ,
(SEQ ID NO: 7047)
QERS,
(SEQ ID NO: 7048)
QNYQ,
(SEQ ID NO: 7049)
QRTC,
(SEQ ID NO: 7050)
QIGH,
(SEQ ID NO: 7051)
QGAI,
(SEQ ID NO: 7052)
QVPP,
(SEQ ID NO: 7053)
QVQQ,
(SEQ ID NO: 7054)
LMRQ,
(SEQ ID NO: 7055)
QYSV,
(SEQ ID NO: 7056)
QAIT,
(SEQ ID NO: 7057)
QKTL,
(SEQ ID NO: 7058)
QLHH,
(SEQ ID NO: 7059)
QNII,
(SEQ ID NO: 7060)
QGHH,
(SEQ ID NO: 7061)
QSKV,
(SEQ ID NO: 7062)
QLPS,
(SEQ ID NO: 7063)
IGKQ,
(SEQ ID NO: 7064)
QAIH,
(SEQ ID NO: 7065)
QHGL,
(SEQ ID NO: 7066)
QFMC,
(SEQ ID NO: 7067)
QNQM,
(SEQ ID NO: 7068)
QHLQ,
(SEQ ID NO: 7069)
QPAR,
(SEQ ID NO: 7070)
QSLQ,
(SEQ ID NO: 7071)
QSQL,
(SEQ ID NO: 7072)
HSQQ,
(SEQ ID NO: 7073)
QMPS,
(SEQ ID NO: 7074)
QGSL,
(SEQ ID NO: 7075)
QVPA,
(SEQ ID NO: 7076)
HYQQ,
(SEQ ID NO: 7077)
QVPS,
(SEQ ID NO: 7078)
RGEQ,
(SEQ ID NO: 7079)
PGQQ,
(SEQ ID NO: 7080)
LEQQ,
(SEQ ID NO: 7081)
QNQS,
(SEQ ID NO: 7082)
QKVI,
(SEQ ID NO: 7083)
QNND,
(SEQ ID NO: 7084)
QSVH,
(SEQ ID NO: 7085)
QPLG,
(SEQ ID NO: 7086)
HNQE,
(SEQ ID NO: 7087)
QIQQ,
(SEQ ID NO: 7088)
QVRN,
(SEQ ID NO: 7089)
PSNQ,
(SEQ ID NO: 7090)
QVGH,
(SEQ ID NO: 7091)
QRDI,
(SEQ ID NO: 7092)
QMPN,
(SEQ ID NO: 7093)
RGLQ,
(SEQ ID NO: 7094)
PSLQ,
(SEQ ID NO: 7095)
QRDQ,
(SEQ ID NO: 7096)
QAKG,
(SEQ ID NO: 7097)
QSAH,
(SEQ ID NO: 7098)
QSTM,
(SEQ ID NO: 7099)
QREM,
(SEQ ID NO: 7100)
QYRA,
(SEQ ID NO: 7101)
QRQQ,
(SEQ ID NO: 7102)
QWQQ,
(SEQ ID NO: 7103)
QRMN,
(SEQ ID NO: 7104)
GDSQ,
(SEQ ID NO: 7105)
QKIS,
(SEQ ID NO: 7106)
PSMQ,
(SEQ ID NO: 7107)
SPRQ,
(SEQ ID NO: 7108)
MEQQ,
(SEQ ID NO: 7109)
QYQN,
(SEQ ID NO: 7110)
QIRQ,
(SEQ ID NO: 7111)
QSVQ,
(SEQ ID NO: 7112)
RSQQ,
(SEQ ID NO: 7113)
QNKL,
(SEQ ID NO: 7114)
QIQH,
(SEQ ID NO: 7115)
PRQQ,
(SEQ ID NO: 7116)
HTQQ,
(SEQ ID NO: 7117)
QRQH,
(SEQ ID NO: 7118)
RNQE,
(SEQ ID NO: 7119)
QSKQ,
(SEQ ID NO: 7120)
QNQP,
(SEQ ID NO: 7121)
QSPQ,
(SEQ ID NO: 7122)
QTRQ,
(SEQ ID NO: 7123)
QNLH,
(SEQ ID NO: 7124)
QNQE,
(SEQ ID NO: 7125)
LNQP,
(SEQ ID NO: 7126)
QNQD,
(SEQ ID NO: 7127)
QNLL,
(SEQ ID NO: 7128)
QLVI,
(SEQ ID NO: 7129)
RTQE,
(SEQ ID NO: 7130)
QTHQ,
(SEQ ID NO: 7131)
QDQH,
(SEQ ID NO: 7132)
QSQH,
(SEQ ID NO: 7133)
VRQQ,
(SEQ ID NO: 7134)
AWQQ,
(SEQ ID NO: 7135)
QSVP,
(SEQ ID NO: 7136)
QNIQ,
(SEQ ID NO: 7137)
LDQQ,
(SEQ ID NO: 7138)
PDQQ,
(SEQ ID NO: 7139)
ESQQ,
(SEQ ID NO: 7140)
QRQL,
(SEQ ID NO: 7141)
QIIV,
(SEQ ID NO: 7142)
QKQS,
(SEQ ID NO: 7143)
QSHQ,
(SEQ ID NO: 7144)
QFVV,
(SEQ ID NO: 7145)
QSQP,
(SEQ ID NO: 7146)
QNEQ,
(SEQ ID NO: 7147)
INQQ,
(SEQ ID NO: 7148)
RNRQ,
(SEQ ID NO: 7149)
RDQK,
(SEQ ID NO: 7150)
QWKR,
(SEQ ID NO: 7151)
ENRQ,
(SEQ ID NO: 7152)
QTQP,
(SEQ ID NO: 7153)
QKQL,
(SEQ ID NO: 7154)
RNQL,
(SEQ ID NO: 7155)
ISIQ,
(SEQ ID NO: 7156)
QTVC,
(SEQ ID NO: 7157)
QQIM,
(SEQ ID NO: 7158)
LNHQ,
(SEQ ID NO: 7159)
QNQA,
(SEQ ID NO: 7160)
QMIH,
(SEQ ID NO: 7161)
RNHQ,
or
(SEQ ID NO:7162)
QKMN;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   42. The AAV particle of any one of embodiments 39-41, wherein [N1]-[N2]-[N3]-[N4] is or comprises:
(i) the amino acid sequence of any of SEQ ID NOs: 1800-2241;
(ii) an amino acid sequence comprising any portion of an amino acid sequence in (i), e.g., any 2, 3, 4, 5, 6, 7, 8, 9, 10, 11, or 12 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   43. The AAV particle of any one of embodiments 37-42, wherein [N1]-[N2]-[N3]-[N4] is or comprises

(SEQ ID NO: 1801)
GSGSPHSKAQNQQ.

44. The AAV particle of any one of embodiments 37-42, wherein [N1]-[N2]-[N3]-[N4] is or comprises

(SEQ ID NO: 1800)
GHDSPHKSGQNQQ.

45. The AAV particle of any one of embodiments 37-42, wherein [N1]-[N2]-[N3]-[N4] is or comprises

(SEQ ID NO: 910)
GSGSPHKYGQNQQT.

46. The AAV particle of any one of embodiments 1-45, wherein the AAV capsid variant comprises an amino acid other than T at position 450 (e.g., S, Y, M, A, C, I, R, L, D, F, V, Q, N, H, E, or G), an amino acid other than I at position 451 (e.g., M, P, E, N, D, S, A, T, G, Q, F, V, L, C, H, R, W, or L), and/or an amino acid other than N at position 452 (e.g., M, E, G, Y, W, T, I, Q, F, V, A, L, I, P, K, R, H, S, D, or S), relative to a reference sequence numbered according to any one of SEQ ID NOs: 36-59, 138, 981 or 982.
47. The AAV particle of any one of embodiments 1-46, wherein the AAV capsid variant comprises the amino acid T at position 450, the amino acid I at position 451, and/or the amino acid N at position 452, relative to a reference sequence numbered according to any one of SEQ ID NOs: 138, 981, or 982.
48. The AAV particle of any one of embodiments 1-47, wherein the AAV capsid variant further comprises [N0], wherein [N0] comprises XA XB and XC, and wherein:
(a) position XA is: T, S, Y, M, A, C, I, R, L, D, F, V, Q, N, H, E, or G;
(b) position XB is: I, M, P, E, N, D, S, A, T, G, Q, F, V, L, C, H, R, W, or L; and
(c) position XC is: N, M, E, G, Y, W, T, I, Q, F, V, A, L, I, P, K, R, H, S, D, or S; and
optionally wherein the AAV capsid variant comprises an amino acid modification, e.g., a conservative substitution, of any of the aforesaid amino acids in (a)-(c).
   49. The AAV particle of embodiment 48, wherein [N0] is or comprises TIN, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TII, TFP, TEK, EIN, TVN, TFN, SIN, TER, TSY, ELH, AIN, SVN, TDN, TFH, TVH, TEN, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAI, AEN, AET, ETA, NNL, or any dipeptide thereof.
   50. The AAV particle of embodiment 48 or 49, wherein [N0]-[N1]-[N2]-[N3]-[N4] is or comprises:
(i) the amino acid sequence of any one of SEQ ID NOs: 2242-2886;
(ii) an amino acid sequence comprising any portion of an amino acid sequence in (i), e.g., any 2, 3, 4, 5, 6, 7, 8, 9, 10, 11, 12, 13, 14, or 15 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   51. The AAV particle of any one of embodiments 48-50, wherein [N0]-[N1]-[N2]-[N3]-[N4] is or comprises TINGSGSPHSKAQNQQ (SEQ ID NO: 2242).
   52. The AAV particle of any one of embodiments 48-50, wherein [N0]-[N1]-[N2]-[N3]-[N4] is or comprises TINGHDSPHKSGQNQQ (SEQ ID NO: 2243).
   53. The AAV particle of any one of embodiments 48-50, wherein [N0]-[N1]-[N2]-[N3]-[N4] is or comprises TINGSGSPHKYGQNQQT (SEQ ID NO: 6551).
   54. The AAV particle of any one of embodiments 1-53, wherein [N1]-[N2]-[N3] is present in loop IV of the AAV capsid variant.
   55. The AAV particle of any one of embodiments 48-54, wherein [N0] and [N4] are present in loop IV of the AAV capsid variant.
   56. The AAV particle of any one of embodiments 48-55, wherein [N0] is present immediately subsequent to position 449, relative to a reference sequence numbered according to the amino acid sequence of SEQ ID NO: 138.
   57. The AAV particle of any one of embodiments 48-55, wherein [N0] is present immediately subsequent to position 449, relative to a reference sequence numbered according to the amino acid sequence of any one of SEQ ID NO: 36-59, 981, or 982.
   58. The AAV particle of any one of embodiments 48-57, wherein [N0] replaces positions 450, 451, and 452 (e.g., T450, I451, and N452), relative to a reference sequence numbered according to SEQ ID NO: 138.
   59. The AAV particle of any one of embodiments 48-58, wherein [N0] replaces positions 450-452 (e.g., T450, I451, and N452), relative to a reference sequence numbered according to any one of SEQ ID NOs: 36-59, 981, or 982.
   60. The AAV particle of any one of embodiments 48-59, wherein [N0] corresponds to positions 450-452 of any one of SEQ ID NOs: 36-59, 138, 981 or 982.
   61. The AAV particle of any one of embodiments 48-60, wherein [N0] is present immediately subsequent to position 449 and wherein [N0] replaces positions 450-452 (e.g., T450, I451, and N452), relative to a reference sequence numbered according to SEQ ID NO: 138.
   62. The AAV particle of any one of embodiments 48-61, wherein [N0] is present immediately subsequent to position 449 and wherein [N0] replaces positions 450-452 (e.g., T450, I451, and N452), relative to a reference sequence numbered according to any one of SEQ ID NOs: 36-59, 981 or 982.
   63. The AAV particle of any one of embodiments 1-62, wherein [N1] is present immediately subsequent to position 452, relative to a reference sequence numbered according to the amino acid sequence of SEQ ID NO: 138.
   64. The AAV particle of any one of embodiments 1-63, wherein [N1] is present immediately subsequent to position 452, relative to a reference sequence numbered according to the amino acid sequence of SEQ ID NO: 981 or 982.
   65. The AAV particle of any one of embodiments 1-64, wherein [N1] replaces positions 453-455 (e.g., G453, S454, and G455), relative to a reference sequence numbered according to SEQ ID NO: 138.
   66. The AAV particle of any one of embodiments 1-65, wherein [N1] replaces positions 453 (e.g., G453), relative to a reference sequence numbered according to SEQ ID NO: 138.
   67. The AAV particle of any one of embodiments 1-66, wherein [N1] replaces positions 453-455 (e.g., G453, 5454, and G455), relative to a reference sequence numbered according to SEQ ID NO: 981.
   68. The AAV particle of any one of embodiments 1-66 or 67, wherein [N1] replaces positions 453-455, relative to a reference sequence numbered according to SEQ ID NO: 982.
   69. The AAV particle of any one of embodiments 1-68, wherein [N1] is present immediately subsequent to position 452 and wherein [N1] replaces positions 453-455 (e.g., G453, S454, and G455), relative to a reference sequence numbered according to SEQ ID NO: 138.
   70. The AAV particle of any one of embodiments 1-64, or 66, wherein [N1] is present immediately subsequent to position 452 and wherein [N1] replaces positions 453 (e.g., G453), relative to a reference sequence numbered according to SEQ ID NO: 138.
   71. The AAV particle of any one of embodiments 1-64, 66, or 70, wherein [N1] is present immediately subsequent to position 452 and wherein [N1] replaces positions 453-455, relative to a reference sequence numbered according to SEQ ID NO: 981 or 982.
   72. The AAV particle of any one of embodiments 1-71, wherein [N1] corresponds to positions 453-455 of any one of SEQ ID NOs: 36-59, 981 or 982.
   73. The AAV particle of any one of embodiment 1-72, wherein the AAV capsid variant comprises an amino acid other than S at position 454 and/or an amino acid other than G at position 455, numbered according to SEQ ID NO: 138, 981, or 982.
   74. The AAV particle of any one of embodiments 1-73, wherein the AAV capsid variant comprises the amino acid H at position 454 and the amino acid D at position 455, numbered according to SEQ ID NO: 138.
   75. The AAV particle of any one of embodiments 1-74, wherein the AAV capsid variant comprises a substitution at position 454 (e.g., S454H) and/or a substitution at position 455 (e.g., G455D), numbered according to SEQ ID NO: 138 or 982.
   76. The AAV particle of any one of embodiments 1-75, wherein the AAV capsid variant comprises the amino acid H at position 454 and the amino acid D at position 455, and further comprises the amino acid sequence SPHSKA (SEQ ID NO: 941) immediately subsequent to position 455, relative to a reference sequence numbered according to SEQ ID NO: 138.
   77. The AAV particle of any one of embodiments 1-71, wherein the AAV capsid variant comprises the amino acid H at position 454 and the amino acid D at position 455, relative to a reference sequence numbered according to SEQ ID NO: 982.
   78. The AAV particle of any one of embodiments 1-77, wherein the AAV capsid variant comprises the amino acid H at position 454 and the amino acid D at position 455, and further comprises the amino acid sequence SPHSKA (SEQ ID NO: 941) immediately subsequent to position 455, relative to a reference sequence numbered according to SEQ ID NO: 982.
   79. The AAV particle of any one of embodiments 1-71, wherein the AAV capsid variant comprises the amino acid S at position 454 and the amino acid G at position 455, relative to a reference sequence numbered according to SEQ ID NO: 138.
   80. The AAV particle of any one of embodiments 1-71 or 79, wherein the AAV capsid variant comprises the amino acid S at position 454 and the amino acid G at position 455, and further comprises the amino acid sequence SPHSKA (SEQ ID NO: 941) immediately subsequent to position 455, relative to a reference sequence numbered according to SEQ ID NO: 138.
   81. The AAV particle of any one of embodiments 1-71, 79, or 80, wherein the AAV capsid variant comprises the amino acid S at position 454 and the amino acid G at position 455, relative to a reference sequence numbered according to SEQ ID NO: 981.
   82. The AAV particle of any one of embodiments 1-71 or 79-81, wherein the AAV capsid variant comprises the amino acid S at position 454 and the amino acid G at position 455, and further comprises the amino acid sequence SPHSKA (SEQ ID NO: 941) immediately subsequent to position 455, relative to a reference sequence numbered according to SEQ ID NO: 981.
   83. The AAV particle of any one of embodiments 1-82, wherein [N2] is present immediately subsequent to position 455, relative to a reference sequence numbered according to the amino acid sequence of SEQ ID NO: 138.
   84. The AAV particle of any one of embodiments 1-82, wherein [N2] corresponds to positions 456-458 (e.g., 5456, P457, H458) of SEQ ID NO: 981 or 982.
   85. The AAV particle of any one of embodiments 1-84, wherein [N2] corresponds to positions 456-458 (e.g., S456, P457, H458) of any one of SEQ ID NOs: 36-59.
   86. The AAV particle of any one of embodiments 1-85, wherein [N2]-[N3] is present immediately subsequent to position 455, relative to a reference sequence numbered according to the amino acid sequence of SEQ ID NO: 138.
   87. The AAV particle of any one of embodiments 1-86, wherein [N2] is present immediately subsequent to position 455, relative to a reference sequence numbered according to the amino acid sequence of SEQ ID NO: 981 or 982.
   88. The AAV particle of any one of embodiments 1-87, wherein [N2]-[N3] is present immediately subsequent to position 455, relative to a reference sequence numbered according to the amino acid sequence of SEQ ID NO: 981 or 982.
   89. The AAV particle of any one of embodiments 1-88, wherein [N2]-[N3] corresponds to positions 456-461 (e.g., S456, P457, H458, S459, K460, A461) of SEQ ID NO: 981.
   90. The AAV particle of any one of embodiments 1-88, wherein [N2]-[N3] corresponds to positions 456-461 (e.g., S456, P457, H458, K459, 5460, G461) of SEQ ID NO: 982.
   91. The AAV particle of any one of embodiments 1-90, wherein [N2] is present immediately subsequent to [N1].
   92. The AAV particle of any one of embodiments 1-64, 66, 70, or 71, wherein [N3] is present immediately subsequent to [N2] and replaces positions 454 and 455 (e.g., S454 and G455), numbered according to SEQ ID NO: 138.
   93. The AAV particle of any one of embodiments 1-64, 66, 70, 71, or 92, wherein [N3] is present immediately subsequent to [N1]-[N2] and replaces positions 454 and 455 (e.g., S454 and G455), numbered according to SEQ ID NO: 138.
   94. The AAV particle of any one of embodiments 39-93, wherein [N4] is present immediately subsequent to position 455, relative to a reference sequence numbered according to the amino acid sequence of SEQ ID NO: 138.
   95. The AAV particle of any one of embodiments 39-94, wherein [N4] replaces positions 456-459 (e.g., Q456, N457, Q458, and Q459), relative to a reference sequence numbered according to the amino acid sequence of SEQ ID NO: 138.
   96. The AAV particle of any one of embodiments 39-95, wherein [N4] corresponds to positions 462-465 (e.g., Q462, N463, Q464, Q465) of SEQ ID NO: 981 or 982.
   97. The AAV particle of any one of embodiments 39-96, wherein [N2]-[N3]-[N4] replaces positions 456-459 (e.g., Q456, N457, Q458, and Q459), relative to a reference sequence numbered according to the amino acid sequence of SEQ ID NO: 138.
   98. The AAV particle of any one of embodiments 39-97, wherein [N2]-[N3]-[N4] is present immediately subsequent to position 455, and wherein [N2]-[N3]-[N4] replaces positions 456-459 (e.g., Q456, N457, Q458, and Q459), relative to a reference sequence numbered according to the amino acid sequence of SEQ ID NO: 138.
   99. The AAV particle of any one of embodiments 39-98, wherein [N2]-[N3]-[N4] corresponds to positions 456-465 (e.g., 5456, P457, H458, S459, K460, A461, Q462, N463, Q464, Q465) of SEQ ID NO: 981.
   100. The AAV particle of any one of embodiments 39-99, wherein [N2]-[N3]-[N4] corresponds to positions 456-465 (e.g., S456, P457, H458, K459, 5460, G461, Q462, N463, Q464, Q465) of SEQ ID NO: 982.
   101. The AAV particle of any one of embodiments 39-100, wherein [N2]-[N3]-[N4] corresponds to positions 456-465 of any one of SEQ ID NOs: 36-59.
   102. The AAV particle of any one of embodiments 39-101, wherein [N1]-[N2]-[N3]-[N4] replaces positions 453-459 (e.g., G453, 5454, G455, Q456, N457, Q458, and Q459), relative to a reference sequence numbered according to the amino acid sequence of SEQ ID NO: 138.
   103. The AAV particle of any one of embodiments 39-102, wherein [N1]-[N2]-[N3]-[N4] is present immediately subsequent to position 452, and wherein [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138.
   104. The AAV particle of any one of embodiments 39-99, 102, or 103, wherein [N1]-[N2]-[N3]-[N4] corresponds to positions 453-465 (e.g., G453, S454, G455, S456, P457, H458, S459, K460, A461, Q462, N463, Q464, Q465) of SEQ ID NO: 981.
   105. The AAV particle of any one of embodiments 39-98 or 100-103, wherein [N1]-[N2]-[N3]-[N4] corresponds to positions 453-465 (e.g., G453, H454, D455, S456, P457, H458, K459, S460, G461, Q462. N463, Q464, Q465) of SEQ ID NO: 982.
   106. The AAV particle of any one of embodiments 39-98, or 101-103, wherein [N1]-[N2]-[N3]-[N4] corresponds to positions 453-465 of any one of SEQ ID NOs: 36-59.
   107. The AAV particle of any one of embodiments 1-98 or 102-104 wherein [N1]-[N2]-[N3] corresponds to positions 453-461 (e.g., G453, S454, G455, S456, P457, H458, S459, K460, A461) of SEQ ID NO: 981.
   108. The AAV particle of any one of embodiments 1-98, 100, 102, 103, or 105, wherein [N1]-[N2]-[N3] corresponds to positions 453-461 (e.g., G453, H454, D455, S456, P457, H458, K459, S460, G461) of SEQ ID NO: 982.
   109. The AAV particle of any one of embodiments 40-98, 101-103, or 106, wherein [N1]-[N2]-[N3] corresponds to positions 453-461 of any one of SEQ ID NOs: 36-59.
   110. The AAV particle of any one of embodiments 39-109, wherein [N0]-[N1]-[N2]-[N3]-[N4] replaces positions 450-459 (e.g., T450, I451. N452, G453, S454, G455, Q456, N457, Q458, and Q459), relative to a reference sequence numbered according to the amino acid sequence of SEQ ID NO: 138.
   111. The AAV particle of any one of embodiments 39-110, wherein [N0]-[N1]-[N2]-[N3]-[N4] is present immediately subsequent to position 449, and wherein [N0]-[N1]-[N2]-[N3]-[N4] replaces positions 450-459 (e.g., T450, I451, N452, G453, S454, G455, Q456, N457, Q458, and Q459), relative to a reference sequence numbered according to the amino acid sequence of SEQ ID NO: 138.
   112. The AAV particle of any one of embodiments 39-99, 102-104, 110 or 111, wherein [N0]-[N1]-[N2]-[N3]-[N4] corresponds to positions 450-465 (e.g., T450, I451, N452, G453, S454, G455, S456, P457, H458, S459, K460, A461, Q462, N463, Q464. Q465) of SEQ ID NO: 981.
   113. The AAV particle of any one of embodiments 39-98, 100-103, or 105-111, wherein [N0]-[N1]-[N2]-[N3]-[N4] corresponds to positions 450-465 (e.g., T450, I451, N452, G453, H454, D455, S456, P457, H458, K459, S460, G461, Q462, N463, Q464. Q465) of SEQ ID NO: 982.
   114. The AAV particle of any one of embodiments 48-98, 101-103, 106, or 109, wherein [N0]-[N1]-[N2]-[N3]-[N4] corresponds to positions 450-465 of any one of SEQ ID NOs: 36-59.
   115. The AAV particle of any one of embodiments 38-99, wherein [N4] replaces positions 462-465 (e.g., Q462, N463, Q464, and Q465), relative to a reference sequence numbered according to the amino acid sequence of SEQ ID NO: 981 or 982.
   116. The AAV particle of any one of embodiments 39-115, wherein [N2]-[N3]-[N4] replaces positions 462-465 (e.g., Q462, N463, Q464, and Q465), relative to a reference sequence numbered according to the amino acid sequence of SEQ ID NO: 981 or 982.
   117. The AAV particle of any one of embodiments 39-116, wherein [N2]-[N3]-[N4] is present immediately subsequent to position 455, and wherein [N2]-[N3]-[N4] replaces positions 462-465 (e.g., Q462, N463, Q464, and Q465), relative to a reference sequence numbered according to the amino acid sequence of SEQ ID NO: 981 or 982.
   118. The AAV particle of any one of embodiments 1-117, wherein [N3] is present immediately subsequent to [N2].
   119. The AAV particle of any one of embodiments 1-118, wherein the AAV capsid variant comprises from N-terminus to C-terminus, [N2]-[N3].
   120. The AAV particle of any one of embodiments 1-119, wherein the AAV capsid variant comprises from N-terminus to C-terminus, [N1]-[N2]-[N3].
   121. The AAV particle of any one of embodiments 28-120, wherein the AAV capsid variant comprises from N-terminus to C-terminus, [N0]-[N1]-[N2]-[N3].
   122. The AAV particle of any one of embodiments 25-121, wherein the AAV capsid variant comprises from N-terminus to C-terminus, [N1]-[N2]-[N3]-[N4].
   123. The AAV particle of any one of embodiments 25-122, wherein the AAV capsid variant comprises from N-terminus to C-terminus, [N0]-[N1]-[N2]-[N3]-[N4].
   124. The AAV particle of any one of the preceding embodiments, wherein the AAV capsid variant comprises an amino acid other T at position 460 (e.g., N, I, C, H, R, L, D, Y, A, M, Q, I, E, K, P, G or S), numbered according to the amino acid sequence of SEQ ID NO: 138.
   125. The AAV particle of any one of the preceding embodiments, wherein the AAV capsid variant comprises the amino acid N, I, C, H, R, L, D, Y, A, M, Q, I, E, K, P, G or S at position 460, numbered according to the amino acid sequence of SEQ ID NO: 138.
   126. The AAV particle of any one of the preceding embodiments, wherein the AAV capsid variant comprises an amino acid other T at position 466 (e.g., N, I, C, H, R, L, D, Y, A, M, Q, I, E, K, P, G or S), numbered according to the amino acid sequence of any one of SEQ ID NOs: 36-59, 981 or 982.
   127. The AAV particle of any one of the preceding embodiments, wherein the AAV capsid variant comprises the amino acid N, I, C, H, R, L, D, Y, A, M, Q, I, E, K, P, G or S at position 466, numbered according to the amino acid sequence of any one of SEQ ID NOs: 36-59, 981 or 982.
   128. The AAV particle of any one of the preceding embodiments, wherein the AAV capsid variant comprises an amino acid other K at position 449 (e.g., an E, an N, or a T), numbered according to the amino acid sequence of any one of SEQ ID NOs: 36-59, 138, 981, or 982.
   129. The AAV particle of any one of the preceding embodiments, wherein the AAV capsid variant comprises the amino E, N, or T at position 449, numbered according to the amino acid sequence of any one of SEQ ID NOs: 36-59, 138, 981 or 982.
   130. An AAV particle comprising an AAV capsid variant and a nucleic acid comprising a transgene encoding an antibody molecule which binds to HER2/neu (e.g., human HER2/neu), wherein the AAV capsid variant comprises an amino acid sequence having the following formula: [N1]-[N2]-[N3], wherein:
(i) [N1] comprises positions X1, X2, and X3, wherein position X2 is S and position X3 is G;
(ii) [N2] comprises the amino acid sequence SPH; and
(iii) [N3] comprises positions X4, X5, and X6, wherein position X5 is K.
   131. The AAV particle of embodiment 130, wherein:
(i) X4 of [N3] is S, T, N, or A; and
(ii) X5 of [N3] is A, V, T, S, G, R, L, or N;
optionally wherein the AAV capsid variant comprises an amino acid modification, e.g., a conservative substitution, of any of the aforesaid amino acids in (i) or (ii).
   132. The AAV particle of embodiment 130 or 131, wherein X4 is S and/or X5 is A.
   133. The AAV particle of any one of embodiments 130-132, wherein [N3] comprises SK, TK, NK, AK, KA, KV, KT, KS, KG, KR, KL, or KN.
   134. The AAV particle of any one of embodiments 130-133, wherein [N3] is or comprises SKA, SKV, SKT, SKS, SKG, SKR, TKA, NKA, SKL, SKN, or AKA.
   135. The AAV particle of any one of embodiments 130-134, wherein [N3] is or comprises SKA.
   136. The AAV particle of any one of embodiments 130-135, wherein [N2]-[N3] comprises SPHSK (SEQ ID NO: 6617), SPHTK (SEQ ID NO: 6641), SPHNK (SEQ ID NO: 6642), or SPHAK (SEQ ID NO: 6643).
   137. The AAV particle of any one of embodiments 130-136, wherein [N2]-[N3] is or comprises:

(i)
(SEQ ID NO: 941)
SPHSKA,
(SEQ ID NO: 6653)
SPHSKV,
(SEQ ID NO: 6647)
SPHSKT,
(SEQ ID NO: 962)
SPHSKS,
(SEQ ID NO: 6648)
SPHSKG,
(SEQ ID NO: 978)
SPHSKR,
(SEQ ID NO: 6655)
SPHTKA,
(SEQ ID NO: 6650)
SPHNKA,
(SEQ ID NO: 960)
SPHSKL,
(SEQ ID NO: 6651)
SPHSKN,
or
(SEQ ID NO: 6652)
SPHAKA;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   138. The AAV particle of any one of embodiments 130-137, wherein [N2]-[N3] is or comprises SPHSKA (SEQ ID NO: 941).
   139. The AAV particle of any one of embodiments 130-138, which comprises an amino acid other than G at position 453 (e.g., M, T, I, E, S, A, N, V, L, K, H, P, R, W, or D), numbered according to SEQ ID NO: 138 or 981.
   140. The AAV particle of any one of embodiments 130-139, which comprises the amino acid G at position 453, numbered according to SEQ ID NO: 138 or 981.
   141. The AAV particle of any one of embodiments 130-140, wherein X1 of [N1] is chosen from: G, M, T, I, E, S, A, N, V, L, K, H, P, R, W, or D; optionally wherein the AAV capsid variant comprises an amino acid modification, e.g., a conservative substitution, of any of the aforesaid amino acids.
   142. The AAV particle of any one of embodiments 130-141, wherein [N1] comprises SG, GS, MS, TS, IS, ES, SS, AS, NS, VS, LS, KS, HS, PS, RS, WS, or DS.
   143. The AAV particle of any one of embodiments 130-142, wherein [N1] is or comprises: GSG, MSG, TSG, ISG, ESG, SSG, ASG, NSG, VSG, LSG, KSG, HSG, PSG, RSG, WSG, or DSG.
   144. The AAV particle of any one of embodiments 130-143, wherein [N1] is or comprises GSG.
   145. The AAV particle of any one of embodiments 130-144, wherein [N1]-[N2] comprises SGSPH (SEQ ID NO: 6668).
   146. The AAV particle of any one of embodiments 130-145, wherein [N1]-[N2] is or comprises:

(i)
(SEQ ID NO: 6700)
GSGSPH,
(SEQ ID NO: 6715)
MSGSPH,
(SEQ ID NO: 6717)
TSGSPH,
(SEQ ID NO: 6718)
ISGSPH,
(SEQ ID NO: 6720)
ESGSPH,
(SEQ ID NO: 6721)
SSGSPH,
(SEQ ID NO: 6723)
ASGSPH,
(SEQ ID NO: 6724)
NSGSPH,
(SEQ ID NO: 6703)
VSGSPH,
(SEQ ID NO: 6725)
LSGSPH,
(SEQ ID NO: 6727)
KSGSPH,
(SEQ ID NO: 6728)
HSGSPH,
(SEQ ID NO: 6730)
PSGSPH,
(SEQ ID NO: 6732)
RSGSPH,
(SEQ ID NO: 6734)
WSGSPH,
(SEQ ID NO: 6735)
DSGSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   147. The AAV particle of any one of embodiments 130-146, wherein [N1]-[N2]-[N3] is or comprises:

(i)
(SEQ ID NO: 6613)
GSGSPHSKA,
(SEQ ID NO: 6873)
GSGSPHSKV,
(SEQ ID NO: 6874)
MSGSPHSKA,
(SEQ ID NO: 6876)
TSGSPHSKA,
(SEQ ID NO: 6877)
ISGSPHSKA,
(SEQ ID NO: 6879)
GSGSPHSKT,
(SEQ ID NO: 6880)
ESGSPHSKA,
(SEQ ID NO: 6881)
SSGSPHSKA,
(SEQ ID NO: 6870)
GSGSPHSKS,
(SEQ ID NO: 6883)
ASGSPHSKA,
(SEQ ID NO: 6884)
NSGSPHSKA,
(SEQ ID NO: 6830)
VSGSPHSKA,
(SEQ ID NO: 6885)
LSGSPHSKA,
(SEQ ID NO: 6887)
KSGSPHSKA,
(SEQ ID NO: 6889)
GSGSPHSKG,
(SEQ ID NO: 6862)
GSGSPHSKR,
(SEQ ID NO: 6890)
HSGSPHSKA,
(SEQ ID NO: 6892)
PSGSPHSKA,
(SEQ ID NO: 6894)
RSGSPHSKA,
(SEQ ID NO: 6895)
GSGSPHTKA,
(SEQ ID NO: 6897)
WSGSPHSKA,
(SEQ ID NO: 6898)
DSGSPHSKA,
(SEQ ID NO: 6900)
GSGSPHNKA,
(SEQ ID NO: 6860)
GSGSPHSKL,
(SEQ ID NO: 6911)
GSGSPHSKN,
or
(SEQ ID NO: 6912)
GSGSPHAKA;

(ii) an amino acid sequence comprising any portion of an amino acid sequence in (i), e.g., any 2, 3, 4, 5, 6, 7, 8, or 9 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   148. The AAV particle of any one of embodiments 130-147, wherein [N1]-[N2]-[N3] is or comprises

(SEQ ID NO: 6613)
GSGSPHSKA.

149. The AAV particle of any one of embodiments 130-148, wherein the AAV capsid variant comprises an amino acid other than Q at position 456 (e.g., R, P, H, L, K, I, G, S, M, or E), an amino acid other than N at position 457 (e.g., D, V, S, P, T, G, Y, W, E, R, H, K, F, A, I, L, or M), an amino acid other than Q at position 458 (e.g., R, L, A, P, H, T, I, F, K, V, M, G, W, Y, S, E, N, or D), an amino acid other than Q at position 459 (e.g., H, K, A, L, P, E, M, I, S, N, R, Y, C, V, T, W, D, G), and/or an amino acid other than T at position 460 (e.g., I, N, S, H, R, L, D, Y, A, or Q), relative to a reference sequence numbered according to SEQ ID NO: 138.
150. The AAV particle of any one of embodiments 130-149, wherein the AAV capsid variant comprises an amino acid other than Q at position 462 (e.g., R, P, H, L, K, I, G, S, M, or E), an amino acid other than N at position 463 (e.g., D, V, S, P, T, G, Y, W, E, R, H, K, F, A, I, L, or M), an amino acid other than Q at position 464 (e.g., R, L, A, P, H, T, I, F, K, V, M, G, W, Y, S, E, N, or D), an amino acid other than Q at position 465 (e.g., H, K, A, L, P, E, M, I, S, N, R, Y, C, V, T, W, D, G), and/or an amino acid other than T at position 466 (e.g., I, N, S, H, R, L, D, Y, A, or Q), relative to a reference sequence numbered according to SEQ ID NO: 981.
151. The AAV particle of any one of embodiments 130-150, wherein the AAV capsid variant comprises the amino acid Q at position 456, the amino acid N at position 457, the amino acid Q at position 458, the amino acid Q at position 459, and/or the amino acid T at position 460, relative to a reference sequence numbered according to SEQ ID NO: 138.
152. The AAV particle of any one of embodiments 130-151, wherein the AAV capsid variant comprises the amino acid Q at position 462, the amino acid N at position 463, the amino acid Q at position 464, the amino acid Q at position 465, and/or the amino acid T at position 466, numbered according to SEQ ID NO: 981
153. The AAV particle of any one of embodiments 130-152, wherein the AAV capsid variant further comprises [N4] wherein [N4] comprises X7, X8, X9, X10, and X11, wherein:
(a) X7 is Q, R, P, H, L, K, I, G, S, M, or E;
(b) X8 is N, D, V, S, P, T, G, Y, W, E, R, H, K, F, A, I, L, or M;
(c) X9 is Q, R, L, A, P, H, T, I, F, K, V, M, G, W, Y, S, E, N, D;
(d) X10 is Q, H, K, A, L, P, E, M, I, S, N, R, Y, C, V, T, W, D, G; and
(e) X11 is T, I, N, S, H, R, L, D, Y, A, Q;
optionally wherein the AAV capsid variant comprises an amino acid modification, e.g., a conservative substitution, of any of the aforesaid amino acids in (a)-(e).
   154. The AAV particle of embodiment 153, wherein [N4] is or comprises:

(i)
(SEQ ID NO: 7163)
QNQQT,
(SEQ ID NO: 7164)
QNRHT,
(SEQ ID NO: 7165)
RDQQT,
(SEQ ID NO: 7166)
PNLQT,
(SEQ ID NO: 7167)
HVRQT,
(SEQ ID NO: 7168)
PNQHT,
(SEQ ID NO: 7169)
QSQQT,
(SEQ ID NO: 7170)
QNQQI,
(SEQ ID NO: 7171)
QPAKT,
(SEQ ID NO: 7172)
QTQQN,
(SEQ ID NO: 7173)
QNLAT,
(SEQ ID NO: 7174)
QNQLT,
(SEQ ID NO: 7175)
QGQQT,
(SEQ ID NO: 7176)
LNRQS,
(SEQ ID NO: 7177)
HNQQT,
(SEQ ID NO: 7178)
QNPPT,
(SEQ ID NO: 7179)
QNLQT,
(SEQ ID NO: 7180)
QYQQT,
(SEQ ID NO: 7181)
QDQET,
(SEQ ID NO: 7182)
QNHQT,
(SEQ ID NO: 7183)
QDQQT,
(SEQ ID NO: 7184)
HWQQT,
(SEQ ID NO: 7185)
PNQQT,
(SEQ ID NO: 7186)
QNQLI,
(SEQ ID NO: 7187)
PEQQT,
(SEQ ID NO: 7188)
QRTMT,
(SEQ ID NO: 7189)
QNQQH,
(SEQ ID NO: 7190)
LHQHT,
(SEQ ID NO: 7191)
QHRIT,
(SEQ ID NO: 7192)
QYIHT,
(SEQ ID NO: 7193)
QKFET,
(SEQ ID NO: 7194)
QFPST,
(SEQ ID NO: 7195)
HNQQR,
(SEQ ID NO: 7196)
QAIKT,
(SEQ ID NO: 7197)
QNRQT,
(SEQ ID NO: 7198)
QYQHT,
(SEQ ID NO: 7199)
QNPQS,
(SEQ ID NO: 7200)
QHQLT,
(SEQ ID NO: 7201)
QSPPT,
(SEQ ID NO: 7202)
QAKLT,
(SEQ ID NO: 7203)
KSQQT,
(SEQ ID NO: 7204)
QDRPT,
(SEQ ID NO: 7205)
QSQQL,
(SEQ ID NO: 7206)
QAFHT,
(SEQ ID NO: 7207)
QKQQD,
(SEQ ID NO: 7208)
QNAQT,
(SEQ ID NO: 7209)
HNQLT,
(SEQ ID NO: 7210)
QNQQY,
(SEQ ID NO: 7211)
QKLNT,
(SEQ ID NO: 7212)
QNVQT,
(SEQ ID NO: 7213)
QAQQT,
(SEQ ID NO: 7214)
QNLQA,
(SEQ ID NO: 7215)
QTPPT,
(SEQ ID NO: 7216)
QYQHA,
(SEQ ID NO: 7217)
QGQQA,
(SEQ ID NO: 7218)
QPPAT,
(SEQ ID NO: 7219)
QERPT,
(SEQ ID NO: 7220)
QDLQT,
(SEQ ID NO: 7221)
QAMHT,
(SEQ ID NO: 7222)
LNQQT,
(SEQ ID NO: 7223)
QHPST,
(SEQ ID NO: 7224)
PGLQT,
(SEQ ID NO: 7225)
QGIRT,
(SEQ ID NO: 7226)
QAPAT,
(SEQ ID NO: 7227)
QSQQI,
(SEQ ID NO: 7228)
QIPPT,
(SEQ ID NO: 7229)
QTQLT,
(SEQ ID NO: 7230)
QAPST,
(SEQ ID NO: 7231)
QNTYA,
(SEQ ID NO: 7232)
QNQHI,
(SEQ ID NO: 7233)
QNHLT,
(SEQ ID NO: 7234)
QIGMT,
(SEQ ID NO: 7235)
LNKQT,
(SEQ ID NO: 7236)
QLQQT,
(SEQ ID NO: 7237)
QRMST,
(SEQ ID NO: 7238)
QGILT,
(SEQ ID NO: 7239)
QDRQT,
(SEQ ID NO: 7240)
RDWQT,
(SEQ ID NO: 7241)
QNTHD,
(SEQ ID NO: 7242)
PNLQI,
(SEQ ID NO: 7243)
QERST,
(SEQ ID NO: 7244)
QNYQT,
(SEQ ID NO: 7245)
QRTCT,
(SEQ ID NO: 7246)
QIGHT,
(SEQ ID NO: 7247)
QGAIT,
(SEQ ID NO: 7248)
QVPPT,
(SEQ ID NO: 7249)
QVQQI,
(SEQ ID NO: 7250)
LMRQT,
(SEQ ID NO: 7251)
QYSVT,
(SEQ ID NO: 7252)
QAITT,
(SEQ ID NO: 7253)
QKTLT,
(SEQ ID NO: 7254)
QNQWT,
(SEQ ID NO: 7255)
QLHHT,
(SEQ ID NO: 7256)
QNIII,
(SEQ ID NO: 7257)
QGHHT,
(SEQ ID NO: 7258)
QSKVT,
(SEQ ID NO: 7259)
QLPST,
(SEQ ID NO: 7260)
IGKQT,
(SEQ ID NO: 7261)
QAIHT,
(SEQ ID NO: 7262)
QHGLT,
(SEQ ID NO: 7263)
QFMCT,
(SEQ ID NO: 7264)
QHLQT,
(SEQ ID NO: 7265)
QNHQN,
(SEQ ID NO: 7266)
QPART,
(SEQ ID NO: 7267)
QSLQT,
(SEQ ID NO: 7268)
QSQLT,
(SEQ ID NO: 7269)
QDRQS,
(SEQ ID NO: 7270)
QMPST,
(SEQ ID NO: 7271)
QGSLT,
(SEQ ID NO: 7272)
QVPAT,
(SEQ ID NO: 7273)
QDKQT,
(SEQ ID NO: 7274)
HYQQT,
(SEQ ID NO: 7275)
QVPST,
(SEQ ID NO: 7276)
RGEQT,
(SEQ ID NO: 7277)
PGQQT,
(SEQ ID NO: 7278)
QSLQI,
(SEQ ID NO: 7279)
LEQQT,
(SEQ ID NO: 7280)
QNQST,
(SEQ ID NO: 7281)
QKVIT,
(SEQ ID NO: 7282)
QNNDQ,
(SEQ ID NO: 7283)
QSVHT,
(SEQ ID NO: 7284)
QPLGT,
(SEQ ID NO: 7285)
HNQET,
(SEQ ID NO: 7286)
QNLQI,
(SEQ ID NO: 7287)
QIQQT,
(SEQ ID NO: 7288)
QVRNT,
(SEQ ID NO: 7289)
PSNQT,
(SEQ ID NO: 7290)
QVGHT,
(SEQ ID NO: 7291)
QRDIT,
(SEQ ID NO: 7292)
QMPNT,
(SEQ ID NO: 7293)
RGLQT,
(SEQ ID NO: 7294)
QKQQT,
(SEQ ID NO: 7295)
PSLQT,
(SEQ ID NO: 7296)
QRDQT,
(SEQ ID NO: 7297)
QAKGT,
(SEQ ID NO: 7298)
QSAHT,
(SEQ ID NO: 7299)
QSTMT,
(SEQ ID NO: 7300)
QREMT,
(SEQ ID NO: 7301)
QYRAT,
(SEQ ID NO: 7302)
QWQQT,
(SEQ ID NO: 7303)
QRMNT,
(SEQ ID NO: 7304)
GDSQT,
(SEQ ID NO: 7305)
QKIST,
(SEQ ID NO: 7306)
PSMQT,
(SEQ ID NO: 7307)
SPRQT,
(SEQ ID NO: 7308)
MEQQT,
(SEQ ID NO: 7309)
QYQNT,
(SEQ ID NO: 7310)
QHQQT,
(SEQ ID NO: 7311)
INQQT,
(SEQ ID NO: 7312)
PNQQH,
(SEQ ID NO: 7313)
ENRQT,
(SEQ ID NO: 7314)
QTQQA,
or
(SEQ ID NO: 7315)
QNQAT;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   155. The AAV particle of embodiment 153 or 154, wherein [N1]-[N2]-[N3]-[N4] is or comprises:
(i) the amino acid sequence of any of SEQ ID NOs: 200 or 2887-3076;
(ii) an amino acid sequence comprising any portion of an amino acid sequence in (i), e.g., any 2, 3, 4, 5, 6, 7, 8, 9, 10, 11, 12, or 13 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   156. The AAV particle of any one of embodiments 153-155, wherein [N1]-[N2]-[N3]-[N4] is or comprises GSGSPHSKAQNQQT (SEQ ID NO: 200).
   157. The AAV particle of any one of embodiments 153-155, wherein [N1]-[N2]-[N3]-[N4] is or comprises VSGSPHSKAQNQQT (SEQ ID NO: 903).
   158. The AAV particle of any one of embodiments 130-157, wherein the AAV capsid variant comprises an amino acid other than K at position 449 (e.g., T, E, or N), T at position 450 (e.g., S, E, A, N, V, Q, or G), an amino acid other than I at position 451 (e.g., F, E, V, L, D, S, C, T, A, N, H, R, G, or W), and/or an amino acid other than N at position 452 (e.g., I, P, K, R, H, S, M, Q, D, T, L, A, Y, V, F, E, W, or G), relative to a reference sequence numbered according to SEQ ID NO: 138.
   159. The AAV particle of any one of embodiments 130-157, wherein the AAV capsid variant comprises the amino acid K at position 449, the amino acid T at position 450, the amino acid I at position 451, and/or the amino acid N at position 452, relative to a reference sequence numbered according to SEQ ID NO: 138 or 981.
   160. The AAV particle of any one of embodiments 130-159, wherein the AAV capsid variant further comprises [N0], wherein [N0] comprises X_{A}, X_{B}, X_{C}, and X_{D}, wherein:
(a) X_{A} is K, T, E, or N;
(b) X_{b} is T, S, E, A, N, V, Q, or G;
(c) X_{C} is I, F, E, V, L, D, S, C, T, A, N, H, R, G, or W; and
(d) X_{D} is N, I, P, K, R, H, S, M, Q, D, T, L, A, Y, V, F, E, W, or G;
optionally wherein the AAV capsid variant comprises an amino acid modification, e.g., a conservative substitution, of any of the aforesaid amino acids in (a)-(d).
   161. The AAV article of embodiment 160, wherein [N0] is or comprises:

(i)
(SEQ ID NO: 7316)
KTII,
(SEQ ID NO: 7317)
KTFP,
(SEQ ID NO: 7318)
KTEK,
(SEQ ID NO: 7319)
KTVN,
(SEQ ID NO: 7320)
KTFN,
(SEQ ID NO: 7321)
KTIN,
(SEQ ID NO: 7322)
TTIN,
(SEQ ID NO: 7323)
KSIN,
(SEQ ID NO: 7324)
KTER,
(SEQ ID NO: 7325)
KELH,
(SEQ ID NO: 7326)
KAIN,
(SEQ ID NO: 7327)
KTDN,
(SEQ ID NO: 7328)
KTFH,
(SEQ ID NO: 7329)
KTSN,
(SEQ ID NO: 7330)
ETIN,
(SEQ ID NO: 7331)
NTIN,
(SEQ ID NO: 7332)
KTEN,
(SEQ ID NO: 7333)
KTSS,
(SEQ ID NO: 7334)
KTCN,
(SEQ ID NO: 7335)
KTEH,
(SEQ ID NO: 7336)
KAEM,
(SEQ ID NO: 7337)
KATN,
(SEQ ID NO: 7338)
KAIK,
(SEQ ID NO: 7339)
KTDK,
(SEQ ID NO: 7340)
KTFK,
(SEQ ID NO: 7341)
KSDQ,
(SEQ ID NO: 7342)
KTEI,
(SEQ ID NO: 7343)
KTID,
(SEQ ID NO: 7344)
KNTN,
(SEQ ID NO: 7345)
KTET,
(SEQ ID NO: 7346)
KTEL,
(SEQ ID NO: 7347)
KNIN,
(SEQ ID NO: 7348)
KTEA,
(SEQ ID NO: 7349)
KTAN,
(SEQ ID NO: 7350)
NTIY,
(SEQ ID NO: 7351)
KTFS,
(SEQ ID NO: 7352)
KTES,
(SEQ ID NO: 7353)
KTTN,
(SEQ ID NO: 7354)
KTED,
(SEQ ID NO: 7355)
KTNN,
(SEQ ID NO: 7356)
KEVH,
(SEQ ID NO: 7357)
KTIS,
(SEQ ID NO: 7358)
KTVR,
(SEQ ID NO: 7359)
KTDR,
(SEQ ID NO: 7360)
ETIK,
(SEQ ID NO: 7361)
KNHI,
(SEQ ID NO: 7362)
KESD,
(SEQ ID NO: 7363)
KTIK,
(SEQ ID NO: 7364)
KTDL,
(SEQ ID NO: 7365)
KTVP,
(SEQ ID NO: 7366)
KTVI,
(SEQ ID NO: 7367)
KAEH,
(SEQ ID NO: 7368)
KNCL,
(SEQ ID NO: 7369)
KTVK,
(SEQ ID NO: 7370)
KNAD,
(SEQ ID NO: 7371)
KTIT,
(SEQ ID NO: 7372)
KNCV,
(SEQ ID NO: 7373)
KNAL,
(SEQ ID NO: 7374)
KVIN,
(SEQ ID NO: 7375)
KTEF,
(SEQ ID NO: 7376)
KTRE,
(SEQ ID NO: 7377)
KQGE,
(SEQ ID NO: 7378)
KSEK,
(SEQ ID NO: 7379)
KNVN,
(SEQ ID NO: 7380)
KGGE,
(SEQ ID NO: 7381)
KEFV,
(SEQ ID NO: 7382)
KSDK,
(SEQ ID NO: 7383)
KTEQ,
(SEQ ID NO: 7384)
KEVQ,
(SEQ ID NO: 7385)
KTEY,
(SEQ ID NO: 7386)
KNCW,
(SEQ ID NO: 7387)
KTDV,
(SEQ ID NO: 7388)
KSDI,
(SEQ ID NO: 7389)
KNSI,
(SEQ ID NO: 7390)
KNSL,
(SEQ ID NO: 7391)
KEVV,
(SEQ ID NO: 7392)
KTEP,
(SEQ ID NO: 7393)
KSEL,
(SEQ ID NO: 7394)
KTWQ,
(SEQ ID NO: 7395)
KTEV,
(SEQ ID NO: 7396)
KAVN,
(SEQ ID NO: 7397)
KGVL,
(SEQ ID NO: 7398)
KTEG,
(SEQ ID NO: 7399)
KTRD,
(SEQ ID NO: 7400)
KTGN,
(SEQ ID NO: 7401)
KNAI,
(SEQ ID NO: 7402)
KAEN,
(SEQ ID NO: 7403)
KAET,
(SEQ ID NO: 7404)
KTVH,
(SEQ ID NO: 7405)
KETA,
(SEQ ID NO: 7406)
KNNL,
(SEQ ID NO: 7407)
EAIN,
(SEQ ID NO: 7408)
KSLN,
(SEQ ID NO: 7409)
KTIP,
or
(SEQ ID NO: 7410)
KTIH;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   162. The AAV particle of embodiment 160 or 161, wherein [N0]-[N1]-[N2]-[N3]-[N4] is or comprises:
(i) the amino acid sequence of any one of SEQ ID NOs: 3239-3526 or 3591-3605;
(ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   163. The AAV particle of any one of embodiments 160-162, wherein [N0]-[N1]-[N2]-[N3]-[N4] is or comprises KTINGSGSPHSKAQNQQT (SEQ ID NO: 7411).
   164. The AAV particle of any one of embodiments 160-162, wherein [N0]-[N1]-[N2]-[N3]-[N4] is or comprises KTERVSGSPHSKAQNQQT (SEQ ID NO: 3589).
   165. An AAV particle comprising an AAV capsid variant and a nucleic acid comprising a transgene encoding an antibody molecule which binds to HER2/neu (e.g., human HER2/neu), wherein the AAV capsid variant comprises an amino acid sequence having the following formula: [N1]-[N2]-[N3], wherein:
(i) [N1] comprises positions X1, X2, and X3, wherein position X2 is an amino acid other than S and position X3 is an amino acid other than G;
(ii) [N2] comprises the amino acid sequence SPH; and
(iii) [N3] comprises positions X4, X5, and X6, wherein position X4 is K.
   166. The AAV particle of embodiment 165, wherein:
(i) X5 of [N3] is S, I, T, R, H, Y, L, or M; and
(ii) X6 of [N3] is G, A, L, E, V, R, W, N, Q, or K;
optionally wherein the AAV capsid variant comprises an amino acid modification, e.g., a conservative substitution, of any of the aforesaid amino acids in (i) or (ii).
   167. The AAV particle of embodiment 165 or 166, wherein X5 is S and/or X₆ is G.
   168. The AAV particle of any one of embodiments 165-167, wherein [N3] comprises KS, KI, KT, KR, KH, KY, KL, KM, SG, IG, TG, RG, SA, SL, SE, SV, SR, SW, SN, HG, YG, SQ, IV, SK, LW, MG, or MA.
   169. The AAV particle of any one of embodiments 165-168, wherein [N3] is or comprises KSG, KIG, KTG, KRG, KSA, KSL, KSE, KSV, KSR, KSW, KSN, KHG, KYG, KSQ, KIV, KSK, KLW, KMG, or KMA.
   170. The AAV particle of any one of embodiments 165-169, wherein [N3] is or comprises KSG.
   171. The AAV particle of any one of embodiments 165-170, wherein [N2]-[N3] comprises SPHKS (SEQ ID NO: 6620), SPHKI (SEQ ID NO: 6629), SPHKT (SEQ ID NO: 6627), SPHKR (SEQ ID NO: 6633), NPHKS (SEQ ID NO: 7412), SPHKH (SEQ ID NO: 6644), SPHKY (SEQ ID NO: 6631), SPHKL (SEQ ID NO: 6630), or SPHKM (SEQ ID NO: 6645).
   172. The AAV particle of any one of embodiments 165-171, wherein [N2]-[N3] is or comprises:

(i)
(SEQ ID NO: 946)
SPHKSG,
(SEQ ID NO: 958)
SPHKIG,
(SEQ ID NO: 6654)
SPHKTG,
(SEQ ID NO: 974)
SPHKRG,
(SEQ ID NO: 7413)
NPHKSG,
(SEQ ID NO: 977)
SPHKSA,
(SEQ ID NO: 6656)
SPHKSL,
(SEQ ID NO: 6657)
SPHKSE,
(SEQ ID NO: 6658)
SPHKSV,
(SEQ ID NO: 951)
SPHKSR,
(SEQ ID NO: 6659)
SPHKSW,
(SEQ ID NO: 6660)
SPHKSN,
(SEQ ID NO: 6661)
SPHKHG,
(SEQ ID NO: 966)
SPHKYG,
(SEQ ID NO: 6662)
SPHKSQ,
(SEQ ID NO: 7414)
SPHKIV,
(SEQ ID NO: 6663)
SPHKSK,
(SEQ ID NO: 6664)
SPHKLW,
(SEQ ID NO: 6666)
SPHKMG,
or
(SEQ ID NO: 6667)
SPHKMA;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   173. The AAV particle of any one of embodiments 165-172, wherein [N2]-[N3] is or comprises SPHKSG (SEQ ID NO: 946).
   174. The AAV particle of any one of embodiments 165-173, wherein the AAV capsid variant comprises an amino acid other than G at position 453 (e.g., A, K, W, R, L, I, M, N, T, E, Q, Y, H, F, or V), numbered according to SEQ ID NO: 138 or 981.
   175. The AAV particle of any one of embodiments 165-173, wherein the AAV capsid variant comprises the amino acid G at position 453, numbered according to SEQ ID NO: 138 or 981.
   176. The AAV particle of any one of embodiments 165-175, wherein:
(i) position X1 of [N1] is G, A, K, W, R, L, I, M, N, T, E, Q, Y, H, F, or V;
(ii) position X2 of [N1] is H, Y, R, Q, N, P, or D;
(iii) position X3 of [N1] is D, E, G, V, or N;
optionally wherein the AAV capsid variant comprises an amino acid modification, e.g., a conservative substitution, of any of the aforesaid amino acids in (i), (ii), or (iii).
   177. The AAV particle of any one of embodiments 165-176, wherein position X2 of [N1] is H and position X3 of [N1] is D.
   178. The AAV particle of any one of embodiments 165-177, wherein position X1 of [N1] is G, position X2 of [N1] is H and position X3 of [N1] is D.
   179. The AAV particle of any one of embodiments 165-178, wherein [N1] comprises GH, HD, GY, GR, GQ, AH, GN, KH, GP, WH, RH, LH, IH, MH, GD, NH, TH, EH, QH, YH, HH, FH, VH, YD, HE, RG, QD, RD, ND, PD, QV, DD, HN, or NG 180. The AAV particle of any one of embodiments 165-179, wherein [N1] is or comprises GHD, GYD, GHE, GRG, GQD, GRD, AHD, GND, KHD, GPD, WHD, RHD, LHD, GQV, IHD, MHD, GDD, GHN, NHD, THD, GNG, EHD, QHD, YHD, HHD, FHD, or VHD.
   181. The AAV particle of any one of embodiments 165-180, wherein [N1] is or comprises GHD.
   182. The AAV particle of any one of embodiments 165-181, wherein [N1]-[N2] comprises HDSPH (SEQ ID NO: 6619).
   183. The AAV particle of any one of embodiments 165-182, wherein [N1]-[N2] is or comprises:

(i)
(SEQ ID NO: 6701)
GHDSPH,
(SEQ ID NO: 6746)
GYDSPH,
(SEQ ID NO: 6710)
GHESPH,
(SEQ ID NO: 6705)
GRGSPH,
(SEQ ID NO: 7415)
GHDNPH,
(SEQ ID NO: 6702)
GQDSPH,
(SEQ ID NO: 6748)
GRDSPH,
(SEQ ID NO: 7416)
AHDSPH,
(SEQ ID NO: 6749)
GNDSPH,
(SEQ ID NO: 7417)
KHDSPH,
(SEQ ID NO: 6750)
GPDSPH,
(SEQ ID NO: 7418)
WHDSPH,
(SEQ ID NO: 7419)
RHDSPH,
(SEQ ID NO: 7420)
LHDSPH,
(SEQ ID NO: 6752)
GQVSPH,
(SEQ ID NO: 7421)
IHDSPH,
(SEQ ID NO: 7422)
MHDSPH,
(SEQ ID NO: 6709)
GDDSPH,
(SEQ ID NO: 6753)
GHNSPH,
(SEQ ID NO: 7423)
NHDSPH,
(SEQ ID NO: 7424)
THDSPH,
(SEQ ID NO: 6722)
GNGSPH,
(SEQ ID NO: 7425)
EHDSPH,
(SEQ ID NO: 7426)
QHDSPH,
(SEQ ID NO: 7427)
YHDSPH,
(SEQ ID NO: 7428)
HHDSPH,
(SEQ ID NO: 7429)
FHDSPH,
or
(SEQ ID NO: 7430)
VHDSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   184. The AAV particle of any one of embodiments 165-183, wherein [N1]-[N2]-[N3] is or comprises:

(i)
(SEQ ID NO: 6615)
GHDSPHKSG,
(SEQ ID NO: 6913)
GHDSPHKIG,
(SEQ ID NO: 6914)
GYDSPHKSG,
(SEQ ID NO: 6915)
GHESPHKSG,
(SEQ ID NO: 6916)
GHDSPHKTG,
(SEQ ID NO: 6917)
GRGSPHKRG,
(SEQ ID NO: 7431)
GHDNPHKSG,
(SEQ ID NO: 6825)
GQDSPHKSG,
(SEQ ID NO: 6857)
GHDSPHKSA,
(SEQ ID NO: 6918)
GHDSPHKSL,
(SEQ ID NO: 6920)
GHDSPHKSE,
(SEQ ID NO: 6921)
GRDSPHKSG,
(SEQ ID NO: 7432)
AHDSPHKSG,
(SEQ ID NO: 6922)
GNDSPHKSV,
(SEQ ID NO: 7433)
AHDSPHKIG,
(SEQ ID NO: 6856)
GHESPHKSA,
(SEQ ID NO: 6923)
GQDSPHKIG,
(SEQ ID NO: 6924)
GHDSPHKSV,
(SEQ ID NO: 6859)
GHDSPHKSR,
(SEQ ID NO: 7434)
KHDSPHKSG,
(SEQ ID NO: 6925)
GPDSPHKIG,
(SEQ ID NO: 6926)
GPDSPHKSG,
(SEQ ID NO: 6927)
GHDSPHKSW,
(SEQ ID NO: 7435)
WHDSPHKSG,
(SEQ ID NO: 7436)
RHDSPHKSG,
(SEQ ID NO: 6928)
GHDSPHKSN,
(SEQ ID NO: 6854)
GHDSPHKRG,
(SEQ ID NO: 6930)
GHDSPHKHG,
(SEQ ID NO: 7437)
LHDSPHKSG,
(SEQ ID NO: 6931)
GQVSPHKSG,
(SEQ ID NO: 7438)
IHDSPHKSG,
(SEQ ID NO: 7439)
MHDSPHKSG,
(SEQ ID NO: 6932)
GDDSPHKSV,
(SEQ ID NO: 6933)
GHNSPHKSG,
(SEQ ID NO: 7440)
NHDSPHKSG,
(SEQ ID NO: 7441)
THDSPHKSG,
(SEQ ID NO: 6934)
GNGSPHKRG,
(SEQ ID NO: 7442)
EHDSPHKSG,
(SEQ ID NO: 6935)
GHDSPHKYG,
(SEQ ID NO: 6936)
GHDSPHKSQ,
(SEQ ID NO: 7443)
QHDSPHKSG,
(SEQ ID NO: 7444)
RHDSPHKIV,
(SEQ ID NO: 7445)
YHDSPHKSG,
(SEQ ID NO: 6937)
GNDSPHKIG,
(SEQ ID NO: 7446)
HHDSPHKSG,
(SEQ ID NO: 6938)
GHDSPHKSK,
(SEQ ID NO: 7447)
FHDSPHKSG,
(SEQ ID NO: 6939)
GHDSPHKLW,
(SEQ ID NO: 7448)
VHDSPHKSG,
(SEQ ID NO: 6941)
GHDSPHKMG,
(SEQ ID NO: 6942)
GHDSPHKMA,
or
(SEQ ID NO: 6855)
GDDSPHKSG;

(ii) an amino acid sequence comprising any portion of an amino acid sequence in (i), e.g., any 2, 3, 4, 5, 6, 7, 8, or 9 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   185. The AAV particle of any one of embodiments 165-184, wherein [N1]-[N2]-[N3] is or comprises GHDSPHKSG (SEQ ID NO: 6615).
   186. The AAV particle of any one of embodiments 165-185, wherein the AAV capsid variant comprises an amino acid other than Q at position 456 (e.g., R, P, H, K, L, V, A, E, or I), an amino acid other than N at position 457 (e.g., I, K, S, H, R, T, D, Y, L, W, F, A, Q, or M), an amino acid other than Q at position 458 (e.g., R, V, K, P, Y, H, L, I, E, or M), an amino acid other than Q at position 459 (e.g., H, L, E, P, W, D, I, V, S, K, R, C, M, or N), and/or an amino acid other than T at position 460 (e.g., A, E, K, S, I, P, G, or N), relative to a reference sequence numbered according to SEQ ID NO: 138.
   187. The AAV particle of any one of embodiments 165-186, wherein the AAV capsid variant comprises an amino acid other than Q at position 462 (e.g., R, P, H, K, L, V, A, E, or I), an amino acid other than N at position 463 (e.g., I, K, S, H, R, T, D, Y, L, W, F, A, Q, or M), an amino acid other than Q at position 464 (e.g., R, V, K, P, Y, H, L, I, E, or M), an amino acid other than Q at position 465 (e.g., H, L, E, P, W, D, I, V, S, K, R, C, M, or N), and/or an amino acid other than T at position 466 (e.g., A, E, K, S, I, P, G, or N), relative to a reference sequence numbered according to SEQ ID NO: 982.
   188. The AAV particle of any one of embodiments 165-187, wherein the AAV capsid variant comprises the amino acid Q at position 456, the amino acid N at position 457, the amino acid Q at position 458, the amino acid Q at position 459, and/or the amino acid T at position 460, relative to a reference sequence numbered according to SEQ ID NO: 138.
   189. The AAV particle of any one of embodiments 165-188, wherein the AAV capsid variant comprises the amino acid Q at position 462, the amino acid N at position 463, the amino acid Q at position 464, the amino acid Q at position 465, and/or the amino acid T at position 466, relative to a reference sequence numbered according to SEQ ID NO: 982.
   190. The AAV particle of any one of embodiments 165-189, wherein the AAV capsid variant further comprises [N4] wherein [N4] comprises X7, X8, X9, X10, and X11, wherein:
(a) X7 is Q, R, P, H, L, K, I, G, S, M, or E;
(b) X8 is N, D, V, S, P, T, G, Y, W, E, R, H, K, F, A, I, L, or M;
(c) X9 is Q, R, L, A, P, H, T, I, F, K, V, M, G, W, Y, S, E, N, D;
(d) X10 is Q, H, K, A, L, P, E, M, I, S, N, R, Y, C, V, T, W, D, G; and
(e) X11 is T, I, N, S, H, R, L, D, Y, A, Q;
optionally wherein the AAV capsid variant comprises an amino acid modification, e.g., a conservative substitution, of any of the aforesaid amino acids in (a)-(e).
   191. The AAV particle of embodiment 190, wherein [N4] is or comprises:

(i)
(SEQ ID NO: 7163)
QNQQT,
(SEQ ID NO: 7449)
QIRQT,
(SEQ ID NO: 7450)
QNQHA,
(SEQ ID NO: 7294)
QKQQT,
(SEQ ID NO: 7451)
QSVQT,
(SEQ ID NO: 7452)
RSQQT,
(SEQ ID NO: 7453)
QNKLE,
(SEQ ID NO: 7454)
QNQQK,
(SEQ ID NO: 7455)
QHQQA,
(SEQ ID NO: 7456)
QIQHT,
(SEQ ID NO: 7457)
PRQQT,
(SEQ ID NO: 7458)
HTQQT,
(SEQ ID NO: 7459)
QRQHT,
(SEQ ID NO: 7169)
QSQQT,
(SEQ ID NO: 7460)
QNQQS,
(SEQ ID NO: 7461)
RNQET,
(SEQ ID NO: 7229)
QTQLT,
(SEQ ID NO: 7462)
KNQQT,
(SEQ ID NO: 7183)
QDQQT,
(SEQ ID NO: 7177)
HNQQT,
(SEQ ID NO: 7174)
QNQLT,
(SEQ ID NO: 7463)
QTQQT,
(SEQ ID NO: 7464)
QTQQI,
(SEQ ID NO: 7465)
QSKQA,
(SEQ ID NO: 7466)
QNQPP,
(SEQ ID NO: 7467)
QSPQT,
(SEQ ID NO: 7244)
QNYQT,
(SEQ ID NO: 7182)
QNHQT,
(SEQ ID NO: 7197)
QNRQT,
(SEQ ID NO: 7468)
QNQQG,
(SEQ ID NO: 7233)
QNHLT,
(SEQ ID NO: 7198)
QYQHT,
(SEQ ID NO: 7254)
QNQWT,
(SEQ ID NO: 7469)
QNQHT,
(SEQ ID NO: 7470)
QTRQT,
(SEQ ID NO: 7471)
QNLHT,
(SEQ ID NO: 7222)
LNQQT,
(SEQ ID NO: 7472)
QNQET,
(SEQ ID NO: 7264)
QHLQT,
(SEQ ID NO: 7473)
LNQPT,
(SEQ ID NO: 7474)
QNQDT,
(SEQ ID NO: 7475)
RNQQT,
(SEQ ID NO: 7476)
QNLLT,
(SEQ ID NO: 7477)
QLVIT,
(SEQ ID NO: 7478)
RTQET,
(SEQ ID NO: 7479)
QTHQT,
(SEQ ID NO: 7480)
QNQPA,
(SEQ ID NO: 7481)
QDQHT,
(SEQ ID NO: 7482)
QSQHT,
(SEQ ID NO: 7483)
RNQQI,
(SEQ ID NO: 7484)
VRQQT,
(SEQ ID NO: 7485)
QNQHS,
(SEQ ID NO: 7486)
AWQQT,
(SEQ ID NO: 7487)
QSVPT,
(SEQ ID NO: 7488)
QNIQP,
(SEQ ID NO: 7489)
QNHLN,
(SEQ ID NO: 7490)
LDQQT,
(SEQ ID NO: 7491)
PDQQS,
(SEQ ID NO: 7492)
ESQQT,
(SEQ ID NO: 7493)
QNKQT,
(SEQ ID NO: 7494)
QRQLT,
(SEQ ID NO: 7495)
QIIVT,
(SEQ ID NO: 7496)
QKQST,
(SEQ ID NO: 7497)
QSHQT,
(SEQ ID NO: 7498)
QFVVT,
(SEQ ID NO: 7179)
QNLQT,
(SEQ ID NO: 7170)
QNQQI,
(SEQ ID NO: 7499)
QSQPT,
(SEQ ID NO: 7500)
QNEQT,
(SEQ ID NO: 7267)
QSLQT,
(SEQ ID NO: 7501)
RNRQT,
(SEQ ID NO: 7502)
QSKQT,
(SEQ ID NO: 7503)
QNPLT,
(SEQ ID NO: 7504)
RDQKT,
(SEQ ID NO: 7505)
HNQQN,
(SEQ ID NO: 7506)
QWKRT,
(SEQ ID NO: 7227)
QSQQI,
(SEQ ID NO: 7213)
QAQQT,
(SEQ ID NO: 7507)
QNHQI,
(SEQ ID NO: 7508)
QNQQA,
(SEQ ID NO: 7509)
QNQLN,
(SEQ ID NO: 7510)
QTQPT,
(SEQ ID NO: 7311)
INQQT,
(SEQ ID NO: 7511)
QKQLT,
(SEQ ID NO: 7512)
RNQLA,
(SEQ ID NO: 7513)
RNQQS,
(SEQ ID NO: 7514)
ISIQT,
(SEQ ID NO: 7515)
QNQQN,
(SEQ ID NO: 7516)
QSQQS,
(SEQ ID NO: 7517)
QTVCT,
(SEQ ID NO: 7518)
QYQQI,
(SEQ ID NO: 7519)
QQIMT,
(SEQ ID NO: 7520)
QNEQS,
(SEQ ID NO: 7521)
LNHQT,
(SEQ ID NO: 7522)
QMIHT,
(SEQ ID NO: 7523)
RNHQS,
(SEQ ID NO: 7524)
QKMNT,
(SEQ ID NO: 7525)
QSQQN,
(SEQ ID NO: 7216)
QYQHA;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   192. The AAV particle of embodiment 190 or 191, wherein [N1]-[N2]-[N3]-[N4] is or comprises:
(i) the amino acid sequence of any of SEQ ID NOs: 201 or 3160-3237;
(ii) an amino acid sequence comprising any portion of an amino acid sequence in (i), e.g., any 2, 3, 4, 5, 6, 7, 8, 9, 10, 11, 12, or 13 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   193. The AAV particle of any one of embodiments 190-192, wherein [N1]-[N2]-[N3]-[N4] is or comprises GHDSPHKSGQNQQT (SEQ ID NO: 201).
   194. The AAV particle of any one of embodiments 165-193, wherein the AAV capsid variant comprises an amino acid other than K at position 449 (e.g., T), T at position 450 (e.g., A, S, I, V, N, E, Y, C, G, W, or Q), an amino acid other than I at position 451 (e.g., E, V, S, T, N, D, C, G, Q, L, P, A), and/or an amino acid other than N at position 452 (e.g., S, Y, I, K, F, T, D, E, G, V, L, A, M, Q, H, P, or R), relative to a reference sequence numbered according to SEQ ID NO: 138 or 982.
   195. The AAV particle of any one of embodiments 165-193, wherein the AAV capsid variant comprises the amino acid K at position 449, the amino acid T at position 450, the amino acid I at position 451, and/or the amino acid N at position 452, relative to a reference sequence numbered according to SEQ ID NO: 138.
   196. The AAV particle of any one of embodiments 165-195, wherein the AAV capsid variant further comprises [N0], wherein [N0] comprises X_{A}, X_{B}, X_{C}, and X_{D}, wherein:
(a) X_{A} is K or T;
(b) X_{b} is T, A, S, I, V, N, E, Y, C, G, W, or Q;
(c) X_{C} is I, E, V, S, T, N, D, C, G, Q, L, P, A; and
(d) X_{D} is N, S, Y, I, K, F, T, D, E, G, V, L, A, M, Q, H, P, or R;
optionally wherein the AAV capsid variant comprises an amino acid modification, e.g., a conservative substitution, of any of the aforesaid amino acids in (a)-(d).
   197. The AAV particle of embodiment 196, wherein [N0] is or comprises:

(i)
(SEQ ID NO: 7326)
KAIN,
(SEQ ID NO: 7321)
KTIN,
(SEQ ID NO: 7352)
KTES,
(SEQ ID NO: 7322)
TTIN,
(SEQ ID NO: 7323)
KSIN,
(SEQ ID NO: 7319)
KTVN,
(SEQ ID NO: 7526)
KSIY,
(SEQ ID NO: 7329)
KTSN,
(SEQ ID NO: 7353)
KTTN,
(SEQ ID NO: 7527)
KIIN,
(SEQ ID NO: 7357)
KTIS,
(SEQ ID NO: 7528)
KAII,
(SEQ ID NO: 7363)
KTIK,
(SEQ ID NO: 7375)
KTEF,
(SEQ ID NO: 7371)
KTIT,
(SEQ ID NO: 7355)
KTNN,
(SEQ ID NO: 7343)
KTID,
(SEQ ID NO: 7529)
KAIS,
(SEQ ID NO: 7530)
KTVD,
(SEQ ID NO: 7531)
KTIE,
(SEQ ID NO: 7398)
KTEG,
(SEQ ID NO: 7374)
KVIN,
(SEQ ID NO: 7396)
KAVN,
(SEQ ID NO: 7532)
KTIY,
(SEQ ID NO: 7327)
KTDN,
(SEQ ID NO: 7334)
KTCN,
(SEQ ID NO: 7533)
KNVV,
(SEQ ID NO: 7346)
KTEL,
(SEQ ID NO: 7534)
KTDA,
(SEQ ID NO: 7395)
KTEV,
(SEQ ID NO: 7393)
KSEL,
(SEQ ID NO: 7535)
KTEM,
(SEQ ID NO: 7383)
KTEQ,
(SEQ ID NO: 7316)
KTII,
(SEQ ID NO: 7536)
KIVN,
(SEQ ID NO: 7318)
KTEK,
(SEQ ID NO: 7332)
KTEN,
(SEQ ID NO: 7537)
KIGN,
(SEQ ID NO: 7538)
KEVM,
(SEQ ID NO: 7539)
KYQV,
(SEQ ID NO: 7348)
KTEA,
(SEQ ID NO: 7337)
KATN,
(SEQ ID NO: 7335)
KTEH,
(SEQ ID NO: 7540)
KTVE,
(SEQ ID NO: 7541)
KAID,
(SEQ ID NO: 7542)
KTIM,
(SEQ ID NO: 7543)
KEVG,
(SEQ ID NO: 7544)
KSEM,
(SEQ ID NO: 7545)
KAQQ,
(SEQ ID NO: 7546)
KCGE,
(SEQ ID NO: 7547)
KASN,
(SEQ ID NO: 7345)
KTET,
(SEQ ID NO: 7548)
KTIG,
(SEQ ID NO: 7549)
KTDP,
(SEQ ID NO: 7550)
KELV,
(SEQ ID NO: 7551)
KELM,
(SEQ ID NO: 7552)
KNEI,
(SEQ ID NO: 7553)
KTPN,
(SEQ ID NO: 7554)
KITN,
(SEQ ID NO: 7555)
KTDI,
(SEQ ID NO: 7556)
KTDQ,
(SEQ ID NO: 7557)
KGIN,
(SEQ ID NO: 7558)
KSEI,
(SEQ ID NO: 7378)
KSEK,
(SEQ ID NO: 7559)
KWSA,
(SEQ ID NO: 7560)
KELA,
(SEQ ID NO: 7561)
KQTQ,
(SEQ ID NO: 7562)
KGAD,
(SEQ ID NO: 7563)
KVGE,
(SEQ ID NO: 7564)
KANE,
(SEQ ID NO: 7565)
KTDT,
(SEQ ID NO: 7566)
KTCI,
(SEQ ID NO: 7567)
KELR,
(SEQ ID NO: 7568)
KCQI,
(SEQ ID NO: 7569)
KGVM,
(SEQ ID NO: 7570)
KACD,
(SEQ ID NO: 7571)
KNEL,
(SEQ ID NO: 7572)
KAAE,
(SEQ ID NO: 7573)
KGQN,
(SEQ ID NO: 7574)
KNEF,
(SEQ ID NO: 7575)
KTSI,
(SEQ ID NO: 7367)
KAEH,
(SEQ ID NO: 7576)
KCDQ,
(SEQ ID NO: 7577)
KEIL,
(SEQ ID NO: 7324)
KTER,
(SEQ ID NO: 7401)
KNAI,
(SEQ ID NO: 7339)
KTDK,
(SEQ ID NO: 7578)
KTPD,
(SEQ ID NO: 7410)
KTIH,
or
(SEQ ID NO: 7342)
KTEI;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   198. The AAV particle of embodiment 196 or 197, wherein [N0]-[N1]-[N2]-[N3]-[N4] is or comprises:
(i) the amino acid sequence of any one of SEQ ID NOs: 3606-3836;
(ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof;
(iii) an amino acid sequence comprising one, two, or three but no more than four modifications, e.g., substitutions (e.g., conservative substitutions), insertions, or deletions, relative to any of the amino acid sequences in (i); or
(iv) an amino acid sequence comprising one, two, or three but no more than four different amino acids, relative to any one of the amino acid sequences in (i).
   199. The AAV particle of any one of embodiments 196-198, wherein [N0]-[N1]-[N2]-[N3]-[N4] is or comprises KTINGHDSPHKSGQNQQT (SEQ ID NO: 7579).
   200. The AAV particle of any one of embodiments 196-198, wherein [N0]-[N1]-[N2]-[N3]-[N4] is or comprises KAEIGHDSPHKSGQNQQT (SEQ ID NO: 1754).
   201. The AAV particle of any one of embodiments 196-198, wherein [N0]-[N1]-[N2]-[N3]-[N4] is or comprises KTEKMSGSPHSKAQNQQT (SEQ ID NO: 3241).
   202. The AAV particle of any one of embodiments 130-201, wherein [N1]-[N2]-[N3] is present in loop IV of the AAV capsid variant.
   203. The AAV particle of any one of embodiments 160-164 or 196-202, wherein [N0] and [N4] are present in loop IV of the AAV capsid variant.
   204. The AAV particle of any one of embodiments 160-164 or 196-203, wherein [N0] is present immediately subsequent to position 448, relative to a reference sequence numbered according to the amino acid sequence of SEQ ID NO: 138, 981, or 982.
   205. The AAV particle of any one of embodiments 160-164 or 196-204, wherein [N0] replaces positions 449-452 (e.g., K449, T450, I451, and N452), relative to a reference sequence numbered according to SEQ ID NO: 138, 981, or 982.
   206. The AAV particle of any one of embodiments 160-164 or 196-205, wherein [N0] is present immediately subsequent to position 448 and wherein [N0] replaces positions 449-452 (e.g., K449, T450, I451, and N452), relative to a reference sequence numbered according to SEQ ID NO: 138, 981, or 982.
   207. The AAV particle of any one of embodiments 160-164 or 196-206, wherein [N0] corresponds to positions 449-452 (e.g., K449, T450, I451, and N452) of SEQ ID NO: 981 or 982.
   208. The AAV particle of any one of embodiments 130-207, wherein [N1] is present immediately subsequent to position 452, relative to a reference sequence numbered according to the amino acid sequence of SEQ ID NO: 138, 981, or 982.
   209. The AAV particle of any one of embodiments 130-208, wherein [N1] replaces positions 453-455 (e.g., G453, S454, and G455), relative to a reference sequence numbered according to SEQ ID NO: 138, 981, or 982.
   210. The AAV particle of any one of embodiments 130-208, wherein [N1] replaces position 453 (e.g., G453), relative to a reference sequence numbered according to SEQ ID NO: 138, 981, or 982.
   211. The AAV particle of any one of embodiments 130-164 or 200-210, wherein:
(i) position X1 of [N1] replaces position 453 (e.g., G453);
(ii) position X2 of [N1] corresponds to position 454 (e.g., 5454); and
(iii) position X3 of [N1] corresponds to position 455 (e.g., G455), wherein (i), (ii), and (iii) are numbered according to SEQ ID NO: 138 or SEQ ID NO: 981.
   212. The AAV particle of any one of embodiments 130-164 or 200-210, wherein:
(i) position X1 of [N1] corresponds to position 453 (e.g., G453);
(ii) position X2 of [N1] corresponds to position 454 (e.g., S454); and
(iii) position X3 of [N1] corresponds to position 455 (e.g., G455), wherein (i), (ii), and (iii) are numbered according to SEQ ID NO: 138 or SEQ ID NO: 981.
   213. The AAV particle of any one of embodiments 165-210, wherein:
(i) position X1 of [N1] corresponds to position 453 (e.g., G453);
(ii) position X2 of [N1] replaces position 454 (e.g., S454); and
(iii) position X3 of [N1] replaces position 455 (e.g., G455), wherein (i), (ii), and (iii) are numbered according to SEQ ID NO: 138 or SEQ ID NO: 982.
   214. The AAV particle of any one of embodiments 165-210, wherein [N1] corresponds to positions 453-455 (e.g., G453, H454, D455) of SEQ ID NO 982.
   215. The AAV particle of any one of embodiments 165-210, wherein [N1] corresponds to positions 453-455 (e.g., G453, S454, G455) of SEQ ID NO: 138 or 981.
   216. The AAV particle of any one of embodiments 130-215, wherein [N2] is present immediately subsequent to position 455, relative to a reference sequence numbered according to the amino acid sequence of SEQ ID NO: 138, 981, or 982.
   217. The AAV particle of any one of embodiments 130-216, wherein [N2] corresponds to positions 456-458 (e.g., S456, P457, and H458) of SEQ ID NO: 981 or 982.
   218. The AAV particle of any one of embodiments 130-216, wherein [N2] corresponds to positions 456-458 (e.g., S456, P457, and H458) of any one of SEQ ID NOs: 36-59.
   219. The AAV particle of any one of embodiments 130-218, wherein [N2] is present immediately subsequent to [N1].
   220. The AAV particle of any one of embodiments 130-219, wherein [N3] corresponds to positions 459-460 (e.g., S459, K460, A461) of SEQ ID NO 981.
   221. The AAV particle of any one of embodiments 130-220, wherein [N3] corresponds to positions 459-460 (e.g., S459, K460, A461) of SEQ ID NO: 36, 38, 39, 40, 41, 42, 43, 44, 45, 46, 47, 48, 49, 50, 51, 52, 53, 54, 55, 57, or 59
   222. The AAV particle of any one of embodiments 130-220, wherein [N2]-[N3] is present immediately subsequent to position 455, relative to a reference sequence numbered according to the amino acid sequence of SEQ ID NO: 138, 981, or 982.
   223. The AAV particle of any one of embodiments 130-164 or 200-222, wherein [N2]-[N3] is present immediately subsequent to position 455, relative to a reference sequence numbered according to the amino acid sequence of SEQ ID NO: 981.
   224. The AAV particle of any one of embodiments 130-164 or 200-223, wherein [N2]-[N3] corresponds to positions 456-461 (e.g., S456, P457, H458, S459, K460, A461) of SEQ ID NO: 981.
   225. The AAV particle of any one of embodiments 130-164 or 200-223, wherein [N2]-[N3] corresponds to positions 456-461 (e.g., S456, P457, H458, S459, K460, A461) of any one of SEQ ID NOs: 36, 38, 39, 40, 41, 42, 43, 44, 45, 46, 47, 48, 49, 50, 51, 52, 53, 54, 55, 57, or 59.
   226. The AAV particle of any one of embodiments 165-222, wherein [N3] corresponds to positions 459-460 (e.g., K459, S460, G461) of SEQ ID NO: 982.
   227. The AAV particle of any one of embodiments 165-219 or 222-226, wherein [N3] corresponds to positions 459-460 (e.g., K459, S460, G461) of SEQ ID NO: 37.
   228. The AAV particle of any one of embodiments 165-222 or 226, wherein [N2]-[N3] is present immediately subsequent to position 455, relative to a reference sequence numbered according to the amino acid sequence of SEQ ID NO: 982.
   229. The AAV particle of any one of embodiments 165-228, wherein [N3] replaces positions 454 and 455 (e.g., S454 and G455), numbered according to SEQ ID NO: 138.
   230. The AAV particle of any one of embodiments 165-229, wherein [N3] is present immediately subsequent to [N2] and replaces positions 454 and 455 (e.g., S454 and G455), numbered according to SEQ ID NO: 138.
   231. The AAV particle of any one of embodiments 165-230, wherein [N3] is present immediately subsequent to [N1]-[N2] and replaces positions 454 and 455 (e.g., S454 and G455), numbered according to SEQ ID NO: 138.
   232. The AAV particle of any one of embodiments 165-222, 226, or 228, wherein [N2]-[N3] corresponds to positions 456-461 (e.g., S456, P457, H458, K459, S460, G461) of SEQ ID NO: 982.
   233. The AAV particle of any one of embodiments 165-222, 226, or 228, wherein [N2]-[N3] corresponds to positions 456-461 (e.g., S456, P457, H458, K459, S460, G461) of SEQ ID NO: 37.
   234. The AAV particle of any one of embodiments 153-164 or 190-233, wherein [N4] is present immediately subsequent to position 455, relative to a reference sequence numbered according to the amino acid sequence of SEQ ID NO: 138.
   235. The AAV particle of any one of embodiments 153-164 or 190-234, wherein [N4] replaces positions 456-460 (e.g., Q456, N457, Q458, Q459, and T460), relative to a reference sequence numbered according to the amino acid sequence of SEQ ID NO: 138.
   236. The AAV particle of any one of embodiments 153-164 or 190-235, wherein [N4] corresponds to positions 462-466 (e.g., Q462, N463, Q464, Q465, and T466) of SEQ ID NO: 981 or 982.
   237. The AAV particle of any one of embodiments 153-164 or 190-235, wherein [N4] corresponds to positions 462-466 of any one of SEQ ID NOs: 36-59.
   238. The AAV particle of any one of embodiments 153-164 or 190-235, wherein [N4] corresponds to positions 456-460 (e.g., Q456, N457, Q458, Q459, and T460) of SEQ ID NO: 138.
   239. The AAV particle of any one of embodiments 153-164 or 190-236, wherein [N2]-[N3]-[N4] replaces positions 456-460 (e.g., Q456, N457, Q458, Q459, and T460), relative to a reference sequence numbered according to the amino acid sequence of SEQ ID NO: 138.
   240. The AAV particle of any one of embodiments 153-164 or 190-239, wherein [N2]-[N3]-[N4] is present immediately subsequent to position 455, and wherein [N2]-[N3]-[N4] replaces positions 456-460 (e.g., Q456, N457, Q458, Q459, and T460), relative to a reference sequence numbered according to the amino acid sequence of SEQ ID NO: 138.
   241. The AAV particle of any one of embodiments 153-164 or 190-240, wherein [N1]-[N2]-[N3]-[N4] replaces positions 453-460 (e.g., G453, S454, G455, Q456, N457, Q458, Q459, and Q460), relative to a reference sequence numbered according to the amino acid sequence of SEQ ID NO: 138.
   242. The AAV particle of any one of embodiments 153-164 or 190-241, wherein [N1]-[N2]-[N3]-[N4] is present immediately subsequent to position 452, and wherein [N1]-[N2]-[N3]-[N4] replaces positions 453-460 (e.g., G453, S454, G455, Q456, N457, Q458, Q459, and Q460), relative to a reference sequence numbered according to the amino acid sequence of SEQ ID NO: 138.
   243. The AAV particle of any one of embodiments 153-164 or 190-242, wherein [N1]-[N2]-[N3]-[N4] corresponds to positions 453-466 (e.g., G453, S454, G455, 5456, P457, H458, S459, K460, A461, Q462, N463, Q464, Q465, Q466) of SEQ ID NO: 981.
   244. The AAV particle of any one of embodiments 153-164 or 190-243, wherein [N1]-[N2]-[N3] corresponds to positions 453-461 (e.g., G453, S454, G455, S456, P457, H458, S459, K460, A461) of SEQ ID NO: 981.
   245. The AAV particle of any one of embodiments 153-164 or 190-242, wherein [N1]-[N2]-[N3]-[N4] corresponds to positions 453-466 (e.g., G453, H454, D455, S456, P457, H458, K459, S460, G461, Q462. N463, Q464, Q465, Q466) of SEQ ID NO: 982.
   246. The AAV particle of any one of embodiments 153-164, 190-242, or 245, wherein [N1]-[N2]-[N3] corresponds to positions 453-461 (e.g., G453, H454, D455, S456, P457, H458, K459, S460, G461) of SEQ ID NO: 982.
   247. The AAV particle of any one of embodiments 153-164, 190-242, or 245, wherein [N1]-[N2]-[N3]-[N4] corresponds to positions 453-466 of any one of SEQ ID NOs: 36-59
   248. The AAV particle of any one of embodiments 160-164 or 190-245, wherein [N0]-[N1]-[N2]-[N3]-[N4] replaces positions 449-460 (e.g., K449, T450, I451, N452, G453, S454, G455, Q456, N457, Q458, Q459, T460), relative to a reference sequence numbered according to the amino acid sequence of SEQ ID NO: 138.
   249. The AAV particle of any one of embodiments 160-164 or 190-248, wherein [N0]-[N1]-[N2]-[N3]-[N4] is present immediately subsequent to position 448, and wherein [N0]-[N1]-[N2]-[N3]-[N4] replaces positions 449-460 (e.g., K449, T450, I451, N452, G453, S454, G455, Q456, N457, Q458, Q459, Q460), relative to a reference sequence numbered according to the amino acid sequence of SEQ ID NO: 138.
   250. The AAV particle of any one of embodiments 160-164 or 202-249, wherein [N0]-[N1]-[N2]-[N3]-[N4] corresponds to positions 449-466 (e.g., K449, T450, I451, N452, G453, S454, G455, S456, P457, H458, S459, K460, A461, Q462, N463, Q464. Q465, T466) of SEQ ID NO: 981.
   251. The AAV particle of any one of embodiments 202-249, wherein [N0]-[N1]-[N2]-[N3]-[N4] corresponds to positions 449-466 (e.g., K449, T450, I451, N452, G453, H454, D455, S456, P457, H458, K459, S460, G461, Q462, N463, Q464, Q465. T466) of SEQ ID NO: 982.
   252. The AAV particle of any one of embodiments 202-249, wherein [N0]-[N1]-[N2]-[N3]-[N4] corresponds to positions 449-466 of any one of SEQ ID NOs: 36-59.
   253. The AAV particle of any one of embodiments 153-164 or 190-251, wherein [N4] is present immediately subsequent to position 461, relative to a reference sequence numbered according to the amino acid sequence of SEQ ID NO: 981 or 982.
   254. The AAV particle of any one of embodiments 153-164 or 190-253, wherein [N4] replaces positions 462-466 (e.g., Q462, N463, Q464, Q465, and T466), relative to a reference sequence numbered according to the amino acid sequence of SEQ ID NO: 981 or 982.
   255. The AAV particle of any one of embodiments 153-164 or 190-154, wherein [N2]-[N3]-[N4] replaces positions 462-466 (e.g., Q462, N463, Q464, Q465, and T466), relative to a reference sequence numbered according to the amino acid sequence of SEQ ID NO: 981 or 982.
   256. The AAV particle of any one of embodiments 153-164 or 190-255, wherein [N2]-[N3]-[N4] is present immediately subsequent to position 455, and wherein [N2]-[N3]-[N4] replaces positions 462-466 (e.g., Q462, N463, Q464, Q465, and T466), relative to a reference sequence numbered according to the amino acid sequence of SEQ ID NO: 981 or 982.
   257. The AAV particle of any one of embodiments 130-256, wherein the AAV capsid variant comprises from N-terminus to C-terminus, [N2]-[N3].
   258. The AAV particle of any one of embodiments 130-257, wherein the AAV capsid variant comprises from N-terminus to C-terminus, [N1]-[N2]-[N3].
   259. The AAV particle of any one of embodiments 130-258, wherein the AAV capsid variant comprises from N-terminus to C-terminus, [N0]-[N1]-[N2]-[N3].
   260. The AAV particle of any one of embodiments 130-259, wherein the AAV capsid variant comprises from N-terminus to C-terminus, [N1]-[N2]-[N3]-[N4].
   261. The AAV particle of any one of embodiments 130-260, wherein the AAV capsid variant comprises from N-terminus to C-terminus, [N0]-[N1]-[N2]-[N3]-[N4]. 262. An AAV particle comprising an AAV capsid variant and a nucleic acid comprising a transgene encoding an antibody molecule which binds to HER2/neu (e.g., human HER2/neu), wherein the AAV capsid variant:
(i) is enriched, e.g., at least about 10, 15, 20, 25, 30, 35, 40, 45, 50, 55, 60, 65, 70, 75, 80, 85, 90, 95, 100, 105, 115, 120, 125, 130, 135, 140, 145, 150, 155, 160, 165, 170, 175, 180, 190, 200, 205, or 210-fold, in the brain of at least two to three species, e.g., a non-human primate and rodent (e.g., mouse), e.g., as compared to a reference sequence of SEQ ID NO: 138, optionally wherein the at least two to three species are Macaca fascicularis, Chlorocebus sabaeus, Callithrix jacchus, and/or mouse (e.g., BALB/c mice, C57Bl/6 mice, and/or CD-1 outbred mice); and/or
(ii) is capable of transducing neuronal and non-neuronal cells, e.g., glial cells, oligodendrocytes (e.g., Olig2 positive oligodendrocytes), and/or astrocytes (e.g., Olig2 positive astrocytes).
   263. An AAV particle comprising an AAV capsid variant and a nucleic acid comprising a transgene encoding an antibody molecule which binds to HER2/neu (e.g., human HER2/neu), wherein the AAV capsid variant comprises:
(a) the amino acid sequence of any of the sequences provided in Tables 1A, 2A, 2B, 9-11, 15, or 16;
(b) an amino acid sequence comprising at least 3, 4, 5, 6, 7, 8, 9, 10, 11, 12, 13, 14, 15, 16, or 17 consecutive amino acids from any one of the sequences provided in Tables 1A, 2A, 2B, 9-11, 15, or 16; or
(c) an amino acid sequence comprising at least one, two, or three but no more than four different amino acids, relative to any one of the sequences provided in Tables 1A, 2A, 2B, 9-11, 15, or 16; or
(d) an amino acid sequence comprising at least one, two, or three but no more than four modifications, e.g., substitutions (e.g., conservative substitutions), relative to the amino acid sequence of any one of the sequences provided in Tables 1A, 2A, 2B, 9-11, 15, or 16.
   264. An AAV particle comprising an AAV capsid variant and a nucleic acid comprising a transgene encoding an antibody molecule which binds to HER2/neu (e.g., human HER2/neu), wherein the AAV capsid variant comprises:
(a) the amino acid sequence of any of SEQ ID NOs: 945-980 or 985-986;
(b) an amino acid sequence comprising at least 3, 4, or 5 consecutive amino acids from any one of SEQ ID NOs: 945-980 or 985-986; or
(c) an amino acid sequence comprising at least one, two, or three but no more than four different amino acids, relative to the amino acid sequence of any one of SEQ ID NOs: 945-980 or 985-986;
(d) an amino sequence comprising at least one, two, or three but no more than four modifications, e.g., substitutions (e.g., conservative substitutions), relative to the amino acid sequence of any one of SEQ ID NOs: 945-980 or 985-986.
   265. An AAV particle comprising an AAV capsid variant and a nucleic acid comprising a transgene encoding an antibody molecule which binds to HER2/neu (e.g., human HER2/neu), wherein the AAV capsid variant comprises:
(a) the amino acid sequence of any of SEQ ID NOs: 2, 200, 201, 941, 943, 204, 208, 404, or 903-909;
(b) an amino acid sequence comprising at least 3, 4, 5, 6, 7, 8, 9, 10, 11, 12, or 13 consecutive amino acids from any one of SEQ ID NOs: 2, 200, 201, 941, 943, 204, 208, 404, or 903-909;
(c) an amino acid sequence comprising at least one, two, or three but no more than four different amino acids, relative to the amino acid sequence of any one of SEQ ID NOs: 2, 200, 201, 941, 943, 204, 208, 404, or 903-909; or
(d) an amino acid sequence comprising at least one, two, or three but no more than four modifications, e.g., substitutions (e.g., conservative substitutions), relative to the amino acid sequence of any one of SEQ ID NOs: 2, 200, 201, 941, 943, 204, 208, 404, or 903-909.
   266. The AAV particle of any one of embodiments 262, 263 or 265, wherein the AAV capsid variant comprises an amino acid sequence comprising at least 4, 5, 6, 7, 8, 9, 10, 11, 12, or 13 consecutive amino acids from any one of SEQ ID NOs: 2, 200, 201, 941, 943, 204, 208, 404, or 903-909.
   267. The AAV particle of any one of embodiments 262-266, wherein the 3 consecutive amino acids comprise SPH.
   268. The AAV particle of any one of embodiments 262-267, wherein the 4 consecutive amino acids comprise SPHS (SEQ ID NO: 6616).
   269. The AAV particle of any one of embodiments 262-268, wherein the 5 consecutive amino acids comprise SPHSK (SEQ ID NO: 6617).
   270. The AAV particle of any one of embodiments 262-269, wherein the 6 consecutive amino acids comprise SPHSKA (SEQ ID NO: 941).
   271. The AAV particle of embodiment 262-266, wherein the 3 consecutive amino acids comprise HDS.
   272. The AAV particle of any one of embodiments 262-266 or 271, wherein the 4 consecutive amino acids comprise HDSP (SEQ ID NO: 6618).
   273. The AAV particle of any one of embodiments 262-266, 271, or 272, wherein the 5 consecutive amino acids comprise HDSPH (SEQ ID NO: 6619).
   274. The AAV particle of any one of embodiments 262-266 or 271-273, wherein the 6 consecutive amino acids comprise HDSPHK (SEQ ID NO: 2).
   275. The AAV particle of any one of embodiments 262-267, wherein:
(i) the 3 consecutive amino acids comprise SPH;
(ii) the 4 consecutive amino acids comprise SPHK (SEQ ID NO: 7580);
(iii) the 5 consecutive amino acids comprise SPHKY (SEQ ID NO: 6631); and/or (iv) the 6 consecutive amino acids comprise SPHKYG (SEQ ID NO: 966).
   276. The AAV particle of embodiment 262, 263, 265, or 266, wherein the AAV capsid variant comprises an amino acid sequence comprising at least one, two, or three but no more than four modifications, e.g., substitutions (e.g., conservative substitutions), relative to the amino acid sequence of any one of SEQ ID NOs: 2, 200, 201, 941, 943, 204, 208, 404, or 903-909.
   277. The AAV particle of any one of embodiments 262-270 or 276, wherein the AAV capsid variant comprises an amino acid sequence comprising at least one, two, or three but no more than four modifications, e.g., substitutions (e.g., conservative substitutions), relative to the amino acid sequence of SPHSKA (SEQ ID NO: 941).
   278. The AAV particle of any one of embodiments 262-266, 271-274, or 276, wherein the AAV capsid variant comprises an amino acid sequence comprising at least one, two, or three but no more than four modifications, e.g., substitutions (e.g., conservative substitutions), relative to the amino acid sequence of

(SEQ ID NO: 2)
HDSPHK.

279. The AAV particle of any one of embodiments 262-267, 275, or 276, wherein the AAV capsid variant comprises an amino acid sequence comprising at least one, two, or three but no more than four modifications, e.g., substitutions (e.g., conservative substitutions), relative to the amino acid sequence of

(SEQ ID NO: 966)
SPHKYG.

280. The AAV particle of embodiment 262 or 263, wherein the AAV capsid variant comprises:
(i) an amino acid sequence comprising at least one, two, or three but no more than four modifications, e.g., substitutions (e.g., conservative substitutions), relative to the amino acid sequence of

(SEQ ID NO: 3589)
KTERVSGSPHSKAQNQQT;

(ii) an amino acid sequence comprising at least one, two, or three but no more than four modifications, e.g., substitutions (e.g., conservative substitutions), relative to the amino acid sequence of

(SEQ ID NO: 1754)
KAEIGHDSPHKSGQNQQT;

(iii) an amino acid sequence comprising at least one, two, or three but no more than four modifications, e.g., substitutions (e.g., conservative substitutions), relative to the amino acid sequence of KTEKMSGSPHSKAQNQQT (SEQ ID NO: 3241);

(SEQ ID NO: 3241)
KTEKMSGSPHSKAQNQQT;

(iv) an amino acid sequence comprising at least one, two, or three but no more than four modifications, e.g., substitutions (e.g., conservative substitutions), relative to the amino acid sequence of KTINGHDSPHSKAQNLQT (SEQ ID NO: 4100); or

(SEQ ID NO: 4100)
KTINGHDSPHSKAQNLQT;

(v) an amino acid sequence comprising at least one, two, or three but no more than four modifications, e.g., substitutions (e.g., conservative substitutions), relative to the amino acid sequence of

(SEQ ID NO: 4062)
KTVNGHDSPHSKAQNQQT.

281. The AAV particle of any one of embodiments 262, 263, 265, 266, or 276, wherein the AAV capsid variant comprises an amino acid sequence comprising at least one, two, or three but no more than four different amino acids relative to the amino acid sequence of any one of SEQ ID NOs: 200, 201, 941, 943, 204, 208, 404, or 903-909.

282. The AAV particle of any one of embodiments 262-270, 276, 277, or 281, wherein the AAV capsid variant comprises an amino acid sequence comprising at least one, two, or three but no more than four different amino acids relative to the amino acid sequence of SPHSKA (SEQ ID NO: 941).
283. The AAV particle of any one of embodiments 262-266, 271-274, 276, 278, or 281, wherein the AAV capsid variant comprises an amino acid sequence comprising at least one, two, or three but no more than four different amino acids relative to the amino acid sequence of HDSPHK (SEQ ID NO: 2).
284. The AAV particle of any one of embodiments 262-267, 275, 276, or 281, wherein the AAV capsid variant comprises an amino acid sequence comprising at least one, two, or three but no more than four different amino acids relative to the amino acid sequence of SPHKYG (SEQ ID NO: 966).
285. The AAV particle of any one of embodiments 262, 263 or 280, wherein the AAV capsid variant comprises:
(i) an amino acid sequence comprising at least one, two, or three but no more than four different amino acids relative to the amino acid sequence of KTERVSGSPHSKAQNQQT (SEQ ID NO: 3589);
(ii) an amino acid sequence comprising at least one, two, or three but no more than four different amino acids relative to the amino acid sequence of KAEIGHDSPHKSGQNQQT (SEQ ID NO: 1754);
(iii) an amino acid sequence comprising at least one, two, or three but no more than four different amino acids relative to the amino acid sequence of KTEKMSGSPHSKAQNQQT (SEQ ID NO: 3241);
(iv) an amino acid sequence comprising at least one, two, or three but no more than four different amino acids relative to the amino acid sequence of KTINGHDSPHSKAQNLQT (SEQ ID NO: 4100);
(v) an amino acid sequence comprising at least one, two, or three but no more than four different amino acids relative to the amino acid sequence of KTVNGHDSPHSKAQNQQT (SEQ ID NO: 4062); or (vi) an amino acid sequence comprising at least one, two, or three but no more than four different amino acids relative to the amino acid sequence of KTENVSGSPHSKAQNQQT (SEQ ID NO: 3272).
   286. The AAV particle of any one of embodiments 1-129, 262, 263, 265-279, or 281-284, wherein the AAV capsid variant comprises the amino acid sequence of any of SEQ ID NOs: 200, 201, 941, 943, 204, 208, 404, or 903-909.
   287. The AAV particle of any one of embodiments 262, 263, 267-270, 276, 277, 280-282, 285, or 286, wherein the AAV capsid variant comprises the amino acid sequence of ERVSGSPHSKA (SEQ ID NO: 7581), optionally wherein the amino acid sequence is present immediately subsequent to position 450 and replaces positions 451-455, numbered according to SEQ ID NO: 138.
   288. The AAV particle of any one of embodiments 262, 263, 267-270, 276, 277, 280-282, or 285-287, wherein the AAV capsid variant comprises the amino acid sequence of KTERVSGSPHSKAQNQQT (SEQ ID NO: 3589), optionally wherein the amino acid sequence is present immediately subsequent to position 448 and replaces positions 449-460, numbered according to SEQ ID NO: 138.
   289. The AAV particle of any one of embodiments 262, 263, 271-274, 276, 278, 280, 281, 283, 285, or 286, wherein the AAV capsid variant comprises the amino acid sequence of AEIGHDSPHKSG (SEQ ID NO: 7582), optionally wherein the amino acid sequence is present immediately subsequent to position 449 and replaces positions 450-455, numbered according to SEQ ID NO: 138.
   290. The AAV particle of any one of embodiments 262, 263, 271-274, 276, 278, 280, 281, 283, 285, 286, or 289, wherein the AAV capsid variant comprises the amino acid sequence of KAEIGHDSPHKSGQNQQT (SEQ ID NO: 1754), optionally wherein the amino acid sequence is present immediately subsequent to position 448 and replaces positions 449-460, (e.g., K449, T450, I451, N452, G453, S454, G455, Q456, N457, Q458, Q459. T460), numbered according to SEQ ID NO: 138.
   291. The AAV particle of any one of embodiments 262, 263, 271-274, 276, 278, 280, 281, 283, 285, or 286, which comprises the amino acid sequence of EKMSGSPHSKA (SEQ ID NO: 6401), optionally wherein the amino acid sequence is present immediately subsequent to position 450 and replaces positions 451-455 (e.g., I451, N452, G453, S454, G455), numbered according to SEQ ID NO: 138.
   292. The AAV particle of any one of embodiments 262, 263, 271-274, 276, 278, 280, 281, 283, 285, 286, or 291, wherein the AAV capsid variant comprises the amino acid sequence of KTEKMSGSPHSKAQNQQT (SEQ ID NO: 3241), optionally wherein the amino acid sequence is present immediately subsequent to position 448 and replaces positions 449-460 (e.g., K449, T450, I451, N452, G453, S454, G455, Q456, N457, Q458. Q459, T460), numbered according to SEQ ID NO: 138.
   293. The AAV particle of any one of embodiments 262, 263, 271-274, 276, 278, 280, 281, 283, 285, or 286, wherein the AAV capsid variant comprises the amino acid sequence of HDSPHSKAQNL (SEQ ID NO: 6402), optionally wherein the amino acid sequence is present immediately subsequent to position 453 and replaces positions 456-458 (e.g., Q456, N457, Q458), numbered according to SEQ ID NO: 138.
   294. The AAV particle of any one of embodiments 262, 263, 271-274, 276, 278, 280, 281, 283, 285, 286, or 293, wherein the AAV capsid variant comprises the amino acid sequence of KTINGHDSPHSKAQNLQT (SEQ ID NO: 4100), optionally wherein the amino acid sequence is present immediately subsequent to position 448 and replaces positions 449-460 (e.g., K449, T450, I451, N452, G453, S454, G455, Q456, N457, Q458, Q459. T460), numbered according to SEQ ID NO: 138.
   295. The AAV particle of any one of embodiments 262, 263, 271-274, 276, 278, 280, 281, 283, 285, or 286, wherein the AAV capsid variant comprises the amino acid sequence of VNGHDSPHSKA (SEQ ID NO: 6403), optionally wherein the amino acid sequence is present immediately subsequent to position 450 and replaces positions 451-455 (e.g., I451, N452, G453, S454, G455), numbered according to SEQ ID NO: 138.
   296. The AAV particle of any one of embodiments 262, 263, 271-274, 276, 278, 280, 281, 283, 285, 286, or 295, which comprises the amino acid sequence of KTVNGHDSPHSKAQNQQT (SEQ ID NO: 4062), optionally wherein the amino acid sequence is present immediately subsequent to position 448 and replaces positions 449-460 (e.g., K449, T450, I451, N452, G453, S454, G455, Q456, N457, Q458, Q459, T460), numbered according to SEQ ID NO: 138.
   297. The AAV particle of any one of embodiments 262, 263, 271-274, 276, 278, 280, 281, 283, 285, or 286, wherein the AAV capsid variant comprises the amino acid sequence of ENVSGSPHSKA, optionally wherein the amino acid sequence is present immediately subsequent to position 450 and replaces positions 451-455 (e.g., I451, N452, G453, S454, G455), numbered according to SEQ ID NO: 138; or is present at positions 451 to 461 numbered according to SEQ ID NO: 981.
   298. The AAV particle of any one of embodiments 262, 263, 271-274, 276, 278, 280, 281, 283, 285, 286, or 293, wherein the AAV capsid variant comprises the amino acid sequence of KTENVSGSPHSKAQNQQT (SEQ ID NO: 3272), optionally wherein the amino acid sequence is present immediately subsequent to position 448 and replaces positions 449-460 (e.g., K449, T450, I451, N452, G453, S454, G455, Q456, N457, Q458, Q459. T460), numbered according to SEQ ID NO: 138; or is present at positions 449 to 466 numbered according to SEQ ID NO: 981.
   299. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, or 286-288, wherein the AAV capsid variant comprises an amino acid sequence encoded by: the nucleotide sequence of SEQ ID NO: 942; a nucleotide sequence comprising at least one, two, three, four, five, six, or seven modifications, e.g., substitutions (e.g., conservative substitutions), but no more than ten modifications, e.g., substitutions (e.g., conservative substitutions), relative to the nucleotide sequence of SEQ ID NO: 942; or a nucleotide sequence comprising at least one, two, three, four, five, six, or seven, but no more than ten different nucleotides relative to the nucleotide sequence of SEQ ID NO: 942.
   300. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, or 290, wherein the AAV capsid variant comprises an amino acid sequence encoded by: the nucleotide sequence of SEQ ID NO: 3; a nucleotide sequence comprising at least one, two, three, four, five, six, or seven modifications, e.g., substitutions (e.g., conservative substitutions), but no more than ten modifications, e.g., substitutions (e.g., conservative substitutions), relative to the nucleotide sequence of SEQ ID NO: 3; or a nucleotide sequence comprising at least one, two, three, four, five, six, or seven, but no more than ten different nucleotides relative to the nucleotide sequence of SEQ ID NO: 3.
   301. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, or 299, wherein the nucleotide sequence encoding the AAV capsid variant comprises the nucleotide sequence of SEQ ID NO: 942; a nucleotide sequence comprising at least one, two, three, four, five, six, or seven modifications, e.g., substitutions (e.g., conservative substitutions), but no more than ten modifications, e.g., substitutions (e.g., conservative substitutions), relative to the nucleotide sequence of SEQ ID NO: 942; or a nucleotide sequence comprising at least one, two, three, four, five, six, or seven, but no more than ten different nucleotides relative to the nucleotide sequence of SEQ ID NO: 942.
   302. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, or 300, wherein the nucleotide sequence encoding the AAV capsid variant comprises the nucleotide sequence of SEQ ID NO: 3; a nucleotide sequence comprising at least one, two, three, four, five, six, or seven modifications, e.g., substitutions (e.g., conservative substitutions), but no more than ten modifications, e.g., substitutions (e.g., conservative substitutions), relative to the nucleotide sequence of SEQ ID NO: 3; or a nucleotide sequence comprising at least one, two, three, four, five, six, or seven, but no more than ten different nucleotides relative to the nucleotide sequence of SEQ ID NO: 3.
   303. The AAV particle of any one of embodiments 262-302, wherein the amino acid sequence is present in loop IV of the AAV capsid variant.
   304. The AAV particle of any one of embodiments 262-303, wherein the amino acid sequence is present immediately subsequent to position 448, 449, 450, 451, 452, 453, 454, or 455, relative to a reference sequence numbered according to the amino acid sequence of SEQ ID NO: 138.
   305. The AAV particle of any one of embodiments 262-304, wherein the amino acid sequence replaces amino acids 449, 450, 451, 452, 453, 454, 455, 456, 457, 458, 459, and/or 460 (e.g., K449, T450, I451, N452, G453, S454, G455, Q456, N457, Q458, Q459, and/or T460), relative to a reference sequence numbered according to the amino acid sequence of SEQ ID NO: 138.
   306. The AAV particle of any one of embodiments 262-305, wherein the amino acid sequence is present immediately subsequent to position 455, relative to a reference sequence numbered according to the amino acid sequence of SEQ ID NO: 138.
   307. The AAV particle of any one of embodiments 262-306, wherein the amino acid sequence is present immediately subsequent to position 453, relative to a reference sequence numbered according to the amino acid sequence of SEQ ID NO: 138.
   308. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, or 303-307, wherein the AAV capsid variant comprises the amino acid sequence of SPHSKA (SEQ ID NO: 941), wherein the amino acid sequence is present immediately subsequent to position 455, relative to a reference sequence numbered according to the amino acid sequence of SEQ ID NO: 138.
   309. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, or 303-308, wherein the AAV capsid variant comprises the amino acid sequence of SPHSKA (SEQ ID NO: 941), wherein the amino acid sequence is present immediately subsequent to position 455, relative to a reference sequence numbered according to the amino acid sequence of SEQ ID NO: 981.
   310. The AAV particle of embodiment 308 or 309, wherein the AAV capsid variant further comprises an amino acid other than I at position 451, an amino acid other than N at position 452, and an amino acid other than G at position 453, numbered according to any one of SEQ ID NOs: 36, 138, or 981.
   311. The AAV particle of any one of embodiments 308-310, wherein the AAV capsid variant further comprises E at position 451, R at position 452, and V at position 453, numbered according to any one of SEQ ID NOs: 36, 138, or 981.
   312. The AAV particle of any one of embodiments 308-311, wherein the AAV capsid variant further comprises the substitutions I451E, N452R, and G453V, numbered according to any one of SEQ ID NOs: 36, 138, or 981.
   313. The AAV particle of any one of embodiments 308-312, wherein the AAV capsid variant comprises:
(i) E at position 451, R at position 452, and V at position 453, numbered according to any one of SEQ ID NOs: 36, 138, or 981; and
(ii) the amino acid sequence of SPHSKA (SEQ ID NO: 941), wherein the amino acid sequence is present immediately subsequent to position 455, numbered according to any one of SEQ ID NOs: 36, 138, or 981.
   314. The AAV particle of embodiment 308 or 309, which further comprises an amino acid other than I at position 451, an amino acid other than N at position 452, and/or G at position 453, numbered according to SEQ ID NO: 39 or 138.
   315. The AAV particle of any one of embodiments 308, 309, or 314, which further comprises E at position 451, K at position 452, and/or M at position 453, numbered according to SEQ ID NO: 138 or 39.
   316. The AAV particle of any one of embodiments 308, 309, 314, or 315, which further comprises the substitutions I451E, N452K, and G453M, numbered according to SEQ ID NO: 39 or 138.
   317. The AAV particle of any one of embodiments 308, 309, or 314-316, which comprises:
(i) E at position 451, K at position 452, and M at position 453, numbered according to SEQ ID NO: 39 or 138; and
(ii) the amino acid sequence of SPHSKA (SEQ ID NO: 941), wherein the amino acid sequence is present immediately subsequent to position 455, numbered according to SEQ ID NO: 39 or 138.
   318. The AAV particle of embodiment 308 or 309, which further comprises an amino acid other than S at position 454, an amino acid other than G at position 455, and/or Q at position 458, numbered according to SEQ ID NO: 138.
   319. The AAV particle of any one of embodiments 308, 309, or 318, which further comprises H at position 454, D at position 455, and/or L at position 458, numbered according to SEQ ID NO: 138.
   320. The AAV particle of any one of embodiments 308, 309, 318, or 319, which further comprises the substitutions S454H, G455D, and Q458L, numbered according to SEQ ID NO: 138.
   321. The AAV particle of any one of embodiments 308, 309, or 318-320, which comprises:
(i) H at position 454, D at position 455, and/or L at position 458, numbered according to SEQ ID NO: 138; and
(ii) the amino acid sequence of SPHSKA (SEQ ID NO: 941), wherein the amino acid sequence is present immediately subsequent to position 455, numbered according to SEQ ID NO: 138.
   322. The AAV particle of embodiment 308 or 309, which further comprises an amino acid other than I at position 451, an amino acid other than S at position 454, and/or an amino acid other than G at position 455, numbered according to SEQ ID NO: 52 or 138.
   323. The AAV particle of any one of embodiments 308, 309, or 322, which further comprises V at position 451, H at position 454, and/or D at position 455, numbered according to SEQ ID NO: 52 or 138.
   324. The AAV particle of any one of embodiments 308, 309, 322, or 323, wherein the AAV capsid variant further comprises the substitutions I451V, S454H, and/or G455D, numbered according to SEQ ID NO: 52 or 138.
   325. The AAV particle of any one of embodiments 308, 309, or 322-324, wherein the AAV capsid variant comprises:
(i) V at position 451, H at position 454, and/or D at position 455, numbered according to SEQ ID NO: 52 or 138; and
(ii) the amino acid sequence of SPHSKA (SEQ ID NO: 941), wherein the amino acid sequence is present immediately subsequent to position 455, numbered according to SEQ ID NO: 52 or 138.
   326. The AAV particle of embodiment 308 or 309, wherein the AAV capsid variant comprises an amino acid other than I at position 451 and/or G at position 453, numbered according to SEQ ID NO: 138.
   327. The AAV particle of any one of embodiments 308, 309, or 326, wherein the AAV capsid variant comprises E at position 451 and/or V at position 453, numbered according to SEQ ID NO: 138 or 1A.
   328. The AAV particle of any one of embodiments 308, 309, 326, or 327, wherein the AAV capsid variant comprises:
(i) E at position 451 and V at position 453, numbered according to SEQ ID NO: 981 or 138; and
(ii) the amino acid sequence of SPHSKA (SEQ ID NO: 941), wherein the amino acid sequence is present immediately subsequent to position 455, at positions 456-461, numbered according to SEQ ID NO: 981 or 138.
   329. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, wherein the AAV capsid variant comprises the amino acid sequence of HDSPHK (SEQ ID NO: 2), wherein the amino acid sequence is present immediately subsequent to position 453, numbered relative to a reference sequence numbered according to the amino acid sequence of SEQ ID NO: 138.
   330. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, or 329, wherein the AAV capsid variant comprises the amino acid sequence of HDSPHK (SEQ ID NO: 2), wherein the amino acid sequence is present immediately subsequent to position 453, numbered relative to a reference sequence numbered according to the amino acid sequence of SEQ ID NO: 982.
   331. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329, or 330, wherein the AAV capsid variant comprises the amino acid sequence of SPHKSG (SEQ ID NO: 946), wherein the amino acid sequence is present immediately subsequent to position 455, numbered relative to a reference sequence numbered according to the amino acid sequence of SEQ ID NO: 982.
   332. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, or 329-331, wherein the AAV capsid variant comprises:
(i) the amino acid sequence of HDSPHSKA (SEQ ID NO: 4486), which is present immediately subsequent to position 453; and
(ii) a deletion of amino acids SG at position 454 and 455;
wherein (i) and (ii) are numbered according to SEQ ID NO: 138.
   333. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, or 329-332, wherein the AAV capsid variant comprises the amino acids HD at position 454 and 455, and further comprises the amino acid sequence of SPHSKA (SEQ ID NO: 941), which is present immediately subsequent to position 455, numbered relative to SEQ ID NO: 138.
   334. The AAV particle of any one of embodiments 329-333, wherein the AAV capsid variant further comprises an amino acid other than T at position 450, an amino acid other than I at position 451, and an amino acid other than N at position 452, numbered according to SEQ ID NO: 138 or 982.
   335. The AAV particle of any one of embodiments 329-334, wherein the AAV capsid variant further comprises A at position 450, E at position 451, and I at position 452, numbered according to SEQ ID NO: 138 or 982.
   336. The AAV particle of any one of embodiments 329-335, wherein the AAV capsid variant further comprises the substitutions T450A, I451E, and N452I, numbered according to SEQ ID NO: 138 or 982.
   337. The AAV particle of any one of embodiments 329-336, wherein the AAV capsid variant comprises:
(i) A at position 450, E at position 451, and I at position 452, numbered according to SEQ ID NO: 138 or 982; and
(ii) the amino acid sequence of HDSPHK (SEQ ID NO: 2), which is present immediately subsequent to positions 453, numbered according to SEQ ID NO: 138 or 982.
   338. The AAV particle of any one of embodiments 1-22, 25-27, 31, 34-42, 45-50, 53-63, 69, 79, 83-86, 94-98, 102, 103, 110, 111, 118-129, 262-267, 275, 276, 281, 284, 286, or 303-307, wherein the AAV capsid variant comprises the amino acid sequence of SPHKYG (SEQ ID NO: 966), wherein the amino acid sequence is present immediately subsequent to position 455, relative to a reference sequence numbered according to the amino acid sequence of SEQ ID NO: 138.
   339. An AAV particle comprising an AAV capsid variant and a nucleic acid comprising a transgene encoding an antibody molecule which binds to HER2/neu (e.g., human HER2/neu), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 982.
   340. An AAV particle comprising an AAV capsid variant and a nucleic acid comprising a transgene encoding an antibody molecule which binds to HER2/neu (e.g., human HER2/neu), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 981.
   341. An AAV particle comprising an AAV capsid variant and a nucleic acid comprising a transgene encoding an antibody molecule which binds to HER2/neu (e.g., human HER2/neu), wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 37, and optionally further comprising:
(i) one, two, or all of an amino acid other than T at position 450, an amino acid other than I at position 541, and/or an amino acid other than N at position 452, numbered according to SEQ ID NO: 138 or 37;
(ii) one, two, or all of A at position 450, E at position 451, and/or I at position 452, numbered according to SEQ ID NO: 138 or 37.
   342. An AAV particle comprising an AAV capsid variant and a nucleic acid comprising a transgene encoding an antibody molecule which binds to HER2/neu (e.g., human HER2/neu), wherein the AAV capsid variant comprises the amino acid sequence of SPHSKA (SEQ ID NO: 941), wherein the amino acid sequence is present immediately subsequent to position 455, numbered according to the amino acid sequence of any one of SEQ ID NO: 36, 38-55, 57, or 59.
   343. The AAV particle, of any one of the preceding embodiments, wherein the AAV capsid variant further comprises:
(i) a modification, e.g., an insertion, substitution (e.g., conservative substitution), and/or deletion, in loop I, II, VI and/or VIII; and/or
(ii) a substitution at position K449, e.g., a K449R substitution, numbered according to SEQ ID NO: 138.
   344. The AAV particle of any one of the preceding embodiments, wherein the AAV capsid variant comprises:
(i) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 138; or
(ii) an amino acid sequence comprising at least one, two or three, but no more than 30, 20 or 10 different amino acids relative to the amino acid sequence of SEQ ID NO: 138.
   345. The AAV particle of any one of the preceding embodiments, wherein the AAV capsid variant comprises the amino acid sequence of SEQ ID NO: 138, or an amino acid sequence with at least 80% (e.g., at least about 85, 90, 95, 96, 97, 98, or 99%) sequence identity thereto.
   346. The AAV particle of any one of the preceding embodiments, wherein the AAV capsid variant comprises the amino acid sequence of SEQ ID NO: 138.
   347. The AAV particle of any one of the preceding embodiments, wherein the AAV capsid variant comprises an amino acid sequence encoded by the nucleotide sequence of SEQ ID NO: 137, or a sequence with at least 80% (e.g., at least about 85, 90, 95, 96, 97, 98, or 99%) sequence identity thereto.
   348. The AAV particle of any one of the preceding embodiments, wherein the nucleotide sequence encoding the AAV capsid variant comprises the nucleotide sequence of SEQ ID NO: 137, or a sequence with at least 80% (e.g., at least about 85, 90, 95, 96, 97, 98, or 99%) sequence identity thereto.
   349. The AAV particle of any one of the preceding embodiments, wherein the AAV capsid variant comprises a VP1 protein, a VP2 protein, a VP3 protein, or a combination thereof.
   350. The AAV particle of any one of embodiments 1-349, wherein the AAV capsid variant comprises the amino acid sequence corresponding to positions 138-742, e.g., a VP2, of SEQ ID NO: 981 or 982, or a sequence with at least 80% (e.g., at least about 85, 90, 95, 96, 97, 98, or 99%) sequence identity thereto.
   351. The AAV particle of any one of embodiments 1-349, wherein the AAV capsid variant comprises the amino acid sequence corresponding to positions 203-742, e.g., a VP3, of SEQ ID NO: 981 or 982, or a sequence with at least 80% (e.g., at least about 85, 90, 95, 96, 97, 98, or 99%) sequence identity thereto.
   352. The AAV particle of any one of embodiments 1-349, wherein the AAV capsid variant comprises the amino acid sequence corresponding to positions 138-736, e.g., a VP2, of SEQ ID NO: 138, or a sequence with at least 80% (e.g., at least about 85, 90, 95, 96, 97, 98, or 99%) sequence identity thereto.
   353. The AAV particle of any one of embodiments 1-349, wherein the AAV capsid variant comprises the amino acid sequence corresponding to positions 203-736, e.g., a VP3, of SEQ ID NO: 138, or a sequence with at least 80% (e.g., at least about 85, 90, 95, 96, 97, 98, or 99%) sequence identity thereto.
   354. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, or 343, wherein the AAV capsid variant comprises an amino acid sequence comprising at least 3, 4, 5, or 6 consecutive amino acids from the amino acid sequence of SPHSKA (SEQ ID NO: 941), wherein:
(i) the 3 consecutive amino acids comprise SPH;
(ii) the 4 consecutive amino acids comprise SPHS (SEQ ID NO: 6616);
(iii) the 5 consecutive amino acids comprise SPHSK (SEQ ID NO: 6617); or
(iv) the 6 consecutive amino acids comprise SPHSKA (SEQ ID NO: 941);
wherein the AAV capsid variant comprises: (a) a VP1 protein comprising the amino acid sequence of SEQ ID NO: 138 or SEQ ID NO: 981; (b) a VP2 protein comprising the amino acid sequence of positions 138-736 of SEQ ID NO: 138 or positions 138-742 of SEQ ID NO: 981; (c) a VP3 protein comprising the amino acid sequence of positions 203-736 of SEQ ID NO: 138 or positions 203-742 of SEQ ID NO: 981; or (d) an amino acid sequence with at least 90% (e.g., at least about 95, 96, 97, 98, or 99%) sequence identity to any of the amino acid sequences in (a)-(c).
   355. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, 343, or 354, wherein the AAV capsid variant comprises an amino acid sequence comprising at least 3, 4, 5, or 6 consecutive amino acids from the amino acid sequence of SPHSKA (SEQ ID NO: 941), wherein:
(i) the 3 consecutive amino acids comprise SPH;
(ii) the 4 consecutive amino acids comprise SPHS (SEQ ID NO: 6616);
(iii) the 5 consecutive amino acids comprise SPHSK (SEQ ID NO: 6617); or
(iv) the 6 consecutive amino acids comprise SPHSKA (SEQ ID NO: 941);
wherein the AAV capsid variant comprises an amino acid sequence at least 90% (e.g., at least about 95, 96, 97, 98, or 99%) identical to the amino acid sequence of SEQ ID NO: 981.
   356. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, 343, 354, or 355, wherein the AAV capsid variant comprises one or two, but no more than three substitutions relative to the amino acid sequence of SPHSKA (SEQ ID NO: 941), wherein the AAV capsid variant comprises:
(a) a VP1 protein comprising the amino acid sequence of SEQ ID NO: 138 or SEQ ID NO: 981;
(b) a VP2 protein comprising the amino acid sequence of positions 138-736 of SEQ ID NO: 138 or positions 138-742 of SEQ ID NO: 981;
(c) a VP3 protein comprising the amino acid sequence of positions 203-736 of SEQ ID NO: 138 or positions 203-742 of SEQ ID NO: 981; or
(d) an amino acid sequence with at least 90% (e.g., at least about 95, 96, 97, 98, or 99%) sequence identity to any of the amino acid sequences in (a)-(c).
   357. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, 343, or 354-356, wherein the AAV capsid variant comprises one or two, but no more than three substitutions relative to the amino acid sequence of SPHSKA (SEQ ID NO: 941), wherein the AAV capsid variant comprises an amino acid sequence at least 90% (e.g., at least about 95, 96, 97, 98, or 99%) identical to the amino acid sequence of SEQ ID NO: 138 or SEQ ID NO: 981.
   358. The AAV particle of any one of embodiments 354-357, wherein the amino acid sequence is present immediately subsequent to position 455, numbered according to SEQ ID NO: 138 or 981.
   359. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, or 343, wherein the AAV capsid variant comprises an amino acid sequence comprising at least 3, 4, 5, or 6 consecutive amino acids from the amino acid sequence of HDSPHK (SEQ ID NO: 2), wherein:
(i) the 3 consecutive amino acids comprise HDS;
(ii) the 4 consecutive amino acids comprise HDSP (SEQ ID NO: 6618);
(iii) the 5 consecutive amino acids comprise HDSPH (SEQ ID NO: 6619); or
(iv) the 6 consecutive amino acids comprise HDSPHK (SEQ ID NO: 2);
wherein the AAV capsid variant comprises: (a) a VP1 protein comprising the amino acid sequence of SEQ ID NO: 138 or SEQ ID NO: 982; (b) a VP2 protein comprising the amino acid sequence of positions 138-736 of SEQ ID NO: 138 or positions 138-742 of SEQ ID NO: 982; (c) a VP3 protein comprising the amino acid sequence of positions 203-736 of SEQ ID NO: 138 or positions 203-742 of SEQ ID NO: 982; or (d) an amino acid sequence with at least 90% (e.g., at least about 95, 96, 97, 98, or 99%) sequence identity to any of the amino acid sequences in (a)-(c).
   360. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, or 359, wherein the AAV capsid variant comprises an amino acid sequence comprising at least 3, 4, 5, or 6 consecutive amino acids from the amino acid sequence of HDSPHK (SEQ ID NO: 2), wherein:
(i) the 3 consecutive amino acids comprise HDS;
(ii) the 4 consecutive amino acids comprise HDSP (SEQ ID NO: 6618);
(iii) the 5 consecutive amino acids comprise HDSPH (SEQ ID NO: 6619); or
(iv) the 6 consecutive amino acids comprise HDSPHK (SEQ ID NO: 2);
wherein the AAV capsid variant comprises an amino acid sequence at least 90% (e.g., at least about 95, 96, 97, 98, or 99%) identical to the amino acid sequence of SEQ ID NO: 982.
   361. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, 359, or 360, wherein the AAV capsid variant comprises one or two, but no more than three substitutions relative to the amino acid sequence of HDSPHK (SEQ ID NO: 2), wherein the AAV capsid variant comprises:
(a) a VP1 protein comprising the amino acid sequence of SEQ ID NO: 138 or SEQ ID NO: 982;
(b) a VP2 protein comprising the amino acid sequence of positions 138-736 of SEQ ID NO: 138 or positions 138-742 of SEQ ID NO: 982;
(c) a VP3 protein comprising the amino acid sequence of positions 203-736 of SEQ ID NO: 138 or positions 203-742 of SEQ ID NO: 982; or
(d) an amino acid sequence with at least 90% (e.g., at least about 95, 96, 97, 98, or 99%) sequence identity to any of the amino acid sequences in (a)-(c).
   362. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, 359-361, wherein the AAV capsid variant comprises one or two, but no more than three substitutions relative to the amino acid sequence of HDSPHK (SEQ ID NO: 2), wherein the AAV capsid variant comprises an amino acid sequence at least 90% (e.g., at least about 95, 96, 97, 98, or 99%) identical to the amino acid sequence of SEQ ID NO: 982.
   363. The AAV particle of any one of embodiments 359-362, wherein the amino acid sequence is present immediately subsequent to position 453, numbered according to SEQ ID NO: 138 or 982.
   364. The AAV particle of any one of embodiments 1-362, wherein the AAV capsid variant comprises the amino acid sequence of any one of SEQ ID NO: 981 or 982, or an amino acid sequence with at least 80% (e.g., at least about 85, 90, 95, 96, 97, 98, or 99%) sequence identity thereto.
   365. The AAV particle of any one of embodiments 1-364, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 981 or 982.
   366. The AAV particle of any one of embodiments, 1-365, wherein the AAV capsid variant comprises an amino acid sequence comprising at least one, two or three, but not more than 30, 20 or 10 different amino acids, relative to the amino acid sequence of SEQ ID NO: 981 or 982.
   367. The AAV particle of any one of embodiments 1-23, 26-29, 32, 35-43, 46-51, 54-71, 79-89, 94-99, 102-104, 100-112, 115-, 164, 203-, 209, 212, 216-, 224, 234-, 243, 248-, 250, 253-, 270, 276, 277, 281, 282, 286-288, 299, 301, 303-309, 343, 354-357, or 364-366, wherein the AAV capsid variant comprises the amino acid sequence of SEQ ID NO: 981, or an amino acid sequence with at least 80% (e.g., at least about 85, 90, 95, 96, 97, 98, or 99%) sequence identity thereto.
   368. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, 343, 354-357, or 364-367, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 981.
   369. The AAV particle of any one of embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, 343, 354-357, or 364-368, wherein the AAV capsid variant comprises an amino acid sequence comprising at least one, two or three, but not more than 30, 20 or 10 different amino acids, relative to the amino acid sequence of SEQ ID NO: 981.
   370. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, or 359-366, wherein the AAV capsid variant comprises the amino acid sequence of SEQ ID NO: 982, or an amino acid sequence with at least 80% (e.g., at least about 85, 90, 95, 96, 97, 98, or 99%) sequence identity thereto.
   371. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, 359-366, or 370, wherein the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 982.
   372. The AAV particle of any one of embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, 359-366, 370, or 371, wherein the AAV capsid variant comprises an amino acid sequence comprising at least one, two or three, but not more than 30, 20 or 10 different amino acids, relative to the amino acid sequence of SEQ ID NO: 982.
   373. The AAV particle of any one of embodiments 1-372, wherein the AAV capsid variant comprises an amino acid sequence encoded by the nucleotide sequence of SEQ ID NO: 983 or 984, or a nucleotide sequence with at least 80% (e.g., at least about 85, 90, 95, 96, 97, 98, or 99%) sequence identity thereto.
   374. The AAV particle of any one of the preceding embodiments 1-373, wherein the nucleotide sequence encoding the AAV capsid variant comprises the nucleotide sequence of SEQ ID NOs: 983 or 984, or a nucleotide sequence with at least 80% (e.g., at least about 85, 90, 95, 96, 97, 98, or 99%) sequence identity thereto.
   375. The AAV particle of any one of the preceding embodiments 1-23, 26-29, 32, 35-43, 46-51, 54-71, 79-89, 94-99, 102-104, 100-112, 115-164, 203-209, 212, 216-224, 234-243, 248-250, 253-270, 276, 277, 281, 282, 286-288, 299, 301, 303-309, 343, 354-357, 364-369, 373, or 374, wherein the nucleotide sequence encoding the AAV capsid variant comprises the nucleotide sequence of SEQ ID NO: 983, or a nucleotide sequence with at least 80% (e.g., at least about 85, 90, 95, 96, 97, 98, or 99%) sequence identity thereto.
   376. The AAV particle of any one of the preceding embodiments 1-9, 11, 12-22, 24, 26, 28, 30, 33, 35-42, 44, 46-50, 52, 54-78, 83-88, 90-98, 100-103, 105-111, 113-129, 165-211, 213-222, 226-242, 245-249, 251-266, 271-274, 276, 278, 280, 281, 283, 285, 286, 289, 290, 300, 302-307, 329-333, 343, 359-366, 370, or 371-374, wherein the nucleotide sequence encoding the AAV capsid variant comprises the nucleotide sequence of SEQ ID NO: 984, or a nucleotide sequence with at least 80% (e.g., at least about 85, 90, 95, 96, 97, 98, or 99%) sequence identity thereto.
   377. The AAV particle of any one of the preceding embodiments, wherein the nucleotide sequence encoding the AAV capsid variant is codon optimized.
   378. An AAV particle comprising an AAV capsid variant and a nucleic acid comprising a transgene encoding an antibody molecule which binds to HER2/neu (e.g., human HER2/neu), wherein AAV capsid variant comprising the amino acid sequence of SEQ ID NO: 981.
   379. The AAV particle of embodiment 378, wherein the nucleotide sequence encoding the AAV capsid variant comprises the nucleotide sequence of SEQ ID NO: 983, or a nucleotide sequence at least 90%, 95%, or 99% identical thereto.
   380. An AAV particle comprising an AAV capsid variant and a nucleic acid comprising a transgene encoding an antibody molecule which binds to HER2/neu (e.g., human HER2/neu), wherein AAV capsid variant comprising the amino acid sequence of SEQ ID NO: 982.
   381. The AAV particle of embodiment 380, wherein the nucleotide sequence encoding the AAV capsid variant comprises the nucleotide sequence of SEQ ID NO: 984, or a nucleotide sequence at least 90%, 95%, or 99% identical thereto.
   382. An AAV particle comprising an AAV capsid variant and a nucleic acid comprising a transgene encoding an antibody molecule which binds to HER2/neu (e.g., human HER2/neu), wherein the AAV capsid variant comprises the amino acid sequence of any one of SEQ ID NOs: 36-59.
   383. An AAV particle comprising an AAV capsid variant and a nucleic acid comprising a transgene encoding an antibody molecule which binds to HER2/neu (e.g., human HER2/neu), comprising an amino acid sequence encoded by the nucleotide sequence of any one of SEQ ID NOs: 6553-6556 or 16-35, or a nucleotide sequence at least 95% identical thereto.
   384. The AAV particle of embodiment 382 or 383, wherein the nucleotide sequence encoding the AAV capsid variant comprises the nucleotide sequence of any one of SEQ ID NOs: 6553-6556 or 16-35, or a nucleotide sequence at least 95% identical thereto.
   385. The AAV particle of any one of the preceding embodiments, wherein the AAV capsid variant has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.
   386. The AAV particle of any one of embodiments 1-385, wherein the AAV capsid variant transduces a brain region, e.g., a midbrain region (e.g., the hippocampus, or thalamus) or the brain stem, optionally wherein the level of transduction is at least 5, 10, 15, 20, 25, 30, 35, 40, 45, 50, 55, 60, or 65-fold greater as compared to a reference sequence of SEQ ID NO: 138, e.g., when measured by an assay, e.g., an immunohistochemistry assay or a qPCR assay, e.g., as described in Example 11.
   387. The AAV particle of any one of embodiments 1-386, wherein the AAV capsid variant transduces a brain region, e.g., a midbrain region (e.g., the hippocampus, or thalamus) or the brain stem, optionally wherein the level of transduction is at least 30, 35, 40, 45, 50, 55, 60, or 65-fold greater as compared to a reference sequence of SEQ ID NO: 138, e.g., when measured by an assay, e.g., an immunohistochemistry assay or a qPCR assay, e.g., as described in Example 11.
   388. The AAV particle of any one of embodiments 1-387, wherein the AAV capsid variant is enriched at least about 3, 4, 5, 6, 7, 8, 9, or 10-fold, in the brain compared to a reference sequence of SEQ ID NO: 138, e.g., when measured by an assay as described in Example 10.
   389. The AAV particle of any one of embodiments 1-388, wherein the AAV capsid variant is enriched at least about 20, 25, 30, 35, 40, 45, 50, 55, 60, 65, 70, 75, 80 or 85-fold, in the brain compared to a reference sequence of SEQ ID NO: 138, e.g., when measured by an assay as described in Example 10.
   390. The AAV particle of any one of embodiments 1-389, wherein the AAV capsid variant is enriched in the brain of at least two to three species, e.g., a non-human primate and rodent (e.g., mouse), e.g., as compared to a reference sequence of SEQ ID NO: 138.
   391. The AAV particle of any one of embodiments 1-390, wherein the AAV capsid variant is enriched at least about 10, 15, 20, 25, 30, 35, 40, 45, 50, 55, 60, 65, 70, 75, 80, 85, 90, 95, 100, 105, 115, 120, 125, 130, 135, 140, 145, 150, 155, 160, 165, 170, 175, 180, 190, 200, 205, or 210-fold, in the brain of at least two to three species, e.g., a non-human primate and rodent (e.g., mouse), compared to a reference sequence of SEQ ID NO: 138, e.g., when measured by an assay as described in Example 10 or 13.
   392. The AAV particle of embodiment 390 or 391, wherein the at least two to three species are Macaca fascicularis, Chlorocebus sabaeus, Callithrix jacchus, and/or mouse (e.g., BALB/c mice, C57Bl/6 mice, and/or CD-1 outbred mice).
   393. The AAV particle of any one of embodiments 1-392, wherein the AAV capsid variant delivers an increased level of a payload to a brain region, optionally wherein the level of the payload is increased by at least 10, 12, 15, 20, 25, 30, 35, 40, 45, 50, 55, 60, 65, or 70-fold, as compared to a reference sequence of SEQ ID NO: 138, e.g., when measured by an assay, e.g., a qRT-PCR or a qPCR assay (e.g., as described in Example 11 or 17).
   394. The AAV particle of any one of embodiments 1-393, wherein the AAV capsid variant delivers an increased level of viral genomes to a brain region, optionally wherein the level of viral genomes is increased by at least 5, 10, 15, 17, 18, 19, 20, 25, 30, 35, 40, 45, or 50-fold, as compared to a reference sequence of SEQ ID NO: 138, e.g., when measured by an assay, e.g., a qRT-PCR or a qPCR assay (e.g., as described in Example 11 or 17).
   395. The AAV particle of embodiment 393 or 394, wherein the brain region is a midbrain region (e.g., the hippocampus or thalamus), frontal cortex, temporal cortex, motor cortex, cerebral cortex, caudate, putamen, dentate nucleus, substantia nigra, or the brainstem.
   396. The AAV particle of any one of embodiments 1-395, wherein the AAV capsid variant is enriched at least about 5, 10, 15, 20, 25, 30, or 35-fold, in the spinal cord compared to a reference sequence of SEQ ID NO: 138, e.g., when measured by an assay as described in Example 10 or 17, optionally wherein the region of the spinal cord is a thoracic spinal cord region, cervical spinal cord region, C5 ventral horn region, lumbar spinal cord region, or L5 ventral horn region.
   397. The AAV particle of any one of embodiments 1-396, wherein the AAV capsid variant shows preferential transduction in a brain region relative to the transduction in the dorsal root ganglia (DRG).
   398. The AAV particle of any one of embodiments 1-397, wherein the AAV capsid variant shows preferential transduction in a brain region relative to the liver.
   399. The AAV particle of any one of embodiments 1-398, wherein the AAV capsid variant shows preferential transduction in a brain region relative to the transduction in the heart.
   400. The AAV particle of any one of embodiments 1-399, wherein the AAV capsid variant shows preferential transduction in a brain region relative to the transduction in the dorsal root ganglia (DRG) and the heart.
   401. The AAV particle of any one of the preceding embodiments, wherein the AAV capsid variant is capable of transducing non-neuronal cells, e.g., glial cells (e.g., oligodendrocytes or astrocytes).
   402. The AAV particle of embodiment 401, wherein the non-neuronal cells comprise glial cells, oligodendrocytes (e.g., Olig2 positive oligodendrocytes), or astrocytes (e.g., Olig2 positive astrocytes).
   403. The AAV particle of any one of the preceding embodiments, wherein the AAV capsid variant is capable of transducing Olig2 positive cells, e.g., Olig2 positive astrocytes or Olig2 positive oligodendrocytes, e.g., as measured by an assay as described in Example 15.
   404. The recombinant AAV particle of any one of the preceding embodiments, wherein the encoded antibody molecule binds domain I of HER2, domain II of HER2, domain III of HER2, and/or domain IV of HER2.
   405. The recombinant AAV particle of any one of the preceding embodiments, wherein the encoded antibody molecule binds domain IV of HER2.
   406. The recombinant AAV particle of any one of embodiments 1-405, wherein the encoded antibody molecule binds domain IT of HER2.
   407. The AAV particle of any one of the preceding embodiments, wherein the encoded antibody molecule comprises:
(i) a heavy chain variable region comprising one, two, or three HC CDR sequence of any of the CDR sequences of Table 3A-3C; and/or
(ii) a light chain variable region comprising one, two, or three LC CDR sequence of any of the CDR sequences of Table 3A-3C.
   408. The AAV particle of any one of embodiments 1-407, wherein the encoded antibody comprises at least 1-4, e.g., at least one, two, three, or four, modifications, e.g., substitutions, in a HC CDR3 region, e.g., an HC CDR3 according to SEQ ID NO: 5001.
   409. The AAV particle of any one of embodiments 1-408, wherein the encoded heavy chain variable region or the encoded heavy chain comprises:
(i) an amino acid substitution at one, two, three, or all of positions 102 (e.g., D102W), 107 (e.g., M107F), 108 (e.g., D108A), and/or 109 (e.g., Y109L), numbered according to the amino acid sequence of SEQ ID NO: 5001;
(ii) one, two, three, or all of an amino acid other than D at position 102, an amino acid other than M at position 107, an amino acid other than D at position 108, and/or an amino acid other than Y at position 109, numbered according to the amino acid sequence of SEQ ID NO: 5001; or
(iii) one, two, three or all of the amino acid W at position 102, the amino acid F at position 107, A at position 108, and/or L at position 109, numbered according to the amino acid sequence of SEQ ID NO: 5001.
   410. The AAV particle of any one of embodiments 1-409, wherein the encoded heavy chain variable region or the encoded heavy chain comprises:
(a) the amino acid W at position 102, the amino acid F at position 107, the amino acid A position 108, and the amino acid L at position 109, relative to a reference sequence numbered according to the amino acid sequence of SEQ ID NO: 5001;
(b) the amino acid W at position 102, the amino acid F at position 107, and the amino acid A position 108, relative to a reference sequence numbered according to the amino acid sequence of SEQ ID NO: 5001;
(c) the amino acid W at position 102, the amino acid F at position 107, and the amino acid L at position 109, relative to a reference sequence numbered according to the amino acid sequence of SEQ ID NO: 5001;
(d) the amino acid W at position 102, the amino acid A position 108, and the amino acid L at position 109, relative to a reference sequence numbered according to the amino acid sequence of SEQ ID NO: 5001;
(e) the amino acid F at position 107, the amino acid A position 108, and the amino acid L at position 109, relative to a reference sequence numbered according to the amino acid sequence of SEQ ID NO: 5001.
   411. The AAV particle of any one of embodiments 1-410, wherein the encoded heavy chain variable region or the encoded heavy chain comprises:
(i) the amino acid substitutions D98W, M100F, D101A, and Y102L, numbered according to Kabat;
(ii) the amino acid substitutions D98W, M100F, and D101A, numbered according to Kabat;
(iii) the amino acid substitutions D98W, M100F, and Y102L, numbered according to Kabat;
(iv) the amino acid substitutions D98W, D101A, and Y102L, numbered according to Kabat;
(v) the amino acid substitutions M100F, D101A, and Y102L, numbered according to Kabat.
   412. The AAV particle of any one of embodiments 1-411, wherein the encoded heavy chain variable region or the encoded heavy chain comprises an HC CDR1, a HC CDR2, a HC CDR3, wherein:
(i) the HC CDR1, HC CDR2, HC CDR3 comprise the sequences of SEQ ID NO: 5281, 5282, 6510, respectively;
(ii) the HC CDR1, HC CDR2, HC CDR3 comprise the sequences of SEQ ID NO: 5281, 5282, 6515, respectively;
(iii) the HC CDR1, HC CDR2, HC CDR3 comprise the sequences of SEQ ID NO: 5281, 5282, 6520, respectively;
(iv) the HC CDR1, HC CDR2, HC CDR3 comprise the sequences of SEQ ID NO: 5281, 5282, 6525, respectively; or
(v) the HC CDR1, HC CDR2, HC CDR3 comprise the sequences of SEQ ID NO: 5281, 5282, 6530, respectively.
   413. The AAV particle of any one of embodiments 1-412, wherein the encoded antibody comprises:
(i) a heavy chain variable region comprising a HC CDR1 amino acid sequence of SEQ ID NO: 5281, a HC CDR2 amino acid sequence of SEQ ID NO: 5282, and an HC CDR3 amino acid sequence of SEQ ID NO: 5283, 6510, 6515, 6520, 6525 or 6530; and
(ii) a light chain variable region comprising an LC CDR1 amino acid sequence of SEQ ID NO: 5287, an LC CDR2 amino acid sequence of SEQ ID NO: 5288, and an LC CDR3 amino acid sequence of SEQ ID NO: 5289.
   414. The AAV particle of any one of embodiments 1-413, wherein the encoded antibody comprises:
(i) a heavy chain variable region comprising a HC CDR1 amino acid sequence of SEQ ID NO: 5284, a HC CDR2 amino acid sequence of SEQ ID NO: 5285, and an HC CDR3 amino acid sequence of SEQ ID NO: 5286; and
(ii) a light chain variable region comprising an LC CDR1 amino acid sequence of SEQ ID NO: 5287, an LC CDR2 amino acid sequence of SEQ ID NO: 5291, and an LC CDR3 amino acid sequence of SEQ ID NO: 5292;
or a sequence substantially identical (e.g., having at least 70%, 75%, 80%, 85%, 90%, 95%, or 99% sequence identity) to the any of the aforesaid sequences.
   415. The AAV particle of any one of embodiments 1-414, wherein the encoded antibody comprises:
(i) a HC CDR1, a HC CDR2, a HC CDR3, a LC CDR1, a LC CDR2 and an LC CDR3 comprising the amino acid sequences of SEQ ID NO: 5281, 5282, 6510, 5287, 5288, and 5289, respectively;
(ii) a HC CDR1, a HC CDR2, a HC CDR3, a LC CDR1, a LC CDR2 and an LC CDR3 comprising the amino acid sequences of SEQ ID NO: 5281, 5282, 6515, 5287, 5288, and 5289, respectively;
(iii) a HC CDR1, a HC CDR2, a HC CDR3, a LC CDR1, a LC CDR2 and an LC CDR3 comprising the amino acid sequences of SEQ ID NO: 5281, 5282, 6530, 5287, 5288, and 5289, respectively;
(iv) a HC CDR1, a HC CDR2, a HC CDR3, a LC CDR1, a LC CDR2 and an LC CDR3 comprising the amino acid sequences of SEQ ID NO: 5281, 5282, 6530, 5287, 5288, and 5289, respectively.
   416. The AAV particle of any one of embodiments 1-415, wherein the encoded antibody molecule comprises:
(i) a heavy chain variable region (VH) comprising an amino acid sequence of any of the VH sequences of Table 3A-3C, or a sequence having at least 80% (e.g., 85, 90, 95, 96, 97, 98, or 99%) sequence identity to any of the VH sequences of Table 3A-3C; and/or
(ii) a light chain variable region (VL) comprising an amino acid sequence of any of the VL sequences of Table 3A-3C, or a sequence having at least 80% (e.g., 85, 90, 95, 96, 97, 98, or 99%) sequence identity to any of the VL sequences of Table 3A-3C.
   417. The AAV particle of any one of embodiments 1-416, wherein the encoded antibody comprises a heavy chain variable region comprising an amino acid selected from SEQ ID NO: 5001, 5367, 5172, 5106, 5010, 5069, 5192, 5224, 5090, 5110, 5254, 5258, 5130, 5262, 5270, 5326, 6511, 6516, 6521, 6526, 6531, 6536, 6539, 6542, 6545, or 6548, or a sequence at least about 80%, 85%, 90%, 92%, 95%, 97%, 98%, or 99% identical thereto.
   418. The AAV particle of any one of embodiments 1-417, wherein the encoded antibody comprises:
(i) a heavy chain variable region comprising the amino acid sequence of SEQ ID NO: 5270, 6511, 6516, 6521, 6526 or 6531, or a sequence substantially identical (e.g., having at least 70%, 75%, 80%, 85%, 90%, 95%, or 99% sequence identity) thereto; and/or
(i) a light chain variable region comprising the amino acid sequence of SEQ ID NO: 5274, or a sequence substantially identical (e.g., having at least 70%, 75%, 80%, 85%, 90%, 95%, or 99% sequence identity) thereto.
   419. The AAV particle of any one of embodiments 1-418, wherein the encoded antibody molecule comprises:
(i) a heavy chain constant region comprising an amino acid sequence of any of the heavy chain constant region sequences of Table 3A-3C or 20, or a sequence having at least 80% (e.g., 85, 90, 95, 96, 97, 98, or 99%) sequence identity to the heavy chain constant region sequences of Table 3A-3C or 20; and/or
(ii) a light chain constant region (CL) comprising an amino acid sequence of any of the CL sequences of Table 3A-3C or 20, or a sequence having at least 80% (e.g., 85, 90, 95, 96, 97, 98, or 99%) sequence identity to any of the CL sequences of Table 3A-3C or 20.
   420. The AAV particle of any one of embodiments 1-419, wherein the encoded antibody molecule comprises:
(i) a heavy chain comprising an amino acid sequence of any of the heavy chain sequences of Table 3A-3C, or a sequence having at least 80% (e.g., 85, 90, 95, 96, 97, 98, or 99%) sequence identity to any of the heavy chain sequences of Table 3A-3C; and/or
(ii) a light chain comprising an amino acid sequence of any of the light chain sequences of Table 3A-3C, or a sequence having at least 80% (e.g., 85, 90, 95, 96, 97, 98, or 99%) sequence identity to any of the light chain sequences of Table 3A-3C.
   421. The AAV particle of any one of embodiments 1-420, wherein the encoded antibody comprises:
(i) a heavy chain comprising the amino acid sequence of SEQ ID NO: 5272, 7589, 6513, 6518, 6523, 6528, 6533, or a sequence substantially identical (e.g., having at least 70%, 75%, 80%, 85%, 90%, 95%, or 99% sequence identity) thereto; and/or a light chain comprising the amino acid sequence of SEQ ID NO: 5276 or a sequence substantially identical (e.g., having at least 70%, 75%, 80%, 85%, 90%, 95%, or 99% sequence identity) thereto;
(ii) a heavy chain comprising the amino acid sequence of SEQ ID NO: 5272, or a sequence substantially identical (e.g., having at least 70%, 75%, 80%, 85%, 90%, 95%, or 99% sequence identity) thereto; and/or a light chain comprising the amino acid sequence of SEQ ID NO: 5276 or a sequence substantially identical (e.g., having at least 70%, 75%, 80%, 85%, 90%, 95%, or 99% sequence identity) thereto; or
(iii) a heavy chain comprising the amino acid sequence of SEQ ID NO: 7589, or a sequence substantially identical (e.g., having at least 70%, 75%, 80%, 85%, 90%, 95%, or 99% sequence identity) thereto; and/or a light chain comprising the amino acid sequence of SEQ ID NO: 5276 or a sequence substantially identical (e.g., having at least 70%, 75%, 80%, 85%, 90%, 95%, or 99% sequence identity) thereto.
   422. The AAV particle of any one of embodiments 1-421, wherein the encoded antibody molecule that binds to HER2/neu, comprises:
(i) a nucleotide sequence encoding a heavy chain variable region (VH) comprising a nucleotide sequence with at least 90% (e.g., at least about 95, 96, 97, 98, or 99%) sequence identity to the nucleotide sequence of SEQ ID NO: 5269; and
(ii) a nucleotide sequence encoding a light chain variable region (VL) comprising a nucleotide sequence with at least 90% (e.g., at least about 95, 96, 97, 98, or 99%) sequence identity to the nucleotide sequence of SEQ ID NO: 5273 or 5245.
   423. The AAV particle of embodiment 422, wherein the nucleotide sequence encoding the VH comprises the nucleotide sequence of SEQ ID NO: 5269.
   424. The AAV particle of embodiments 422 or 423, wherein the nucleotide sequence encoding the VL comprises the nucleotide sequence of SEQ ID NO: 5273 or 5245.
   425. The AAV particle of embodiments 422-424, wherein the nucleotide sequence encoding the VH comprises the nucleotide sequence of SEQ ID NO: 5269 and the nucleotide sequence encoding the VL comprises the nucleotide sequence of SEQ ID NO: 5273 or 5245.
   426. The AAV particle of any one of embodiments 422-425, wherein the encoded antibody molecule is a full-length antibody, a bispecific antibody, a Fab, a F(ab′)2, a Fv, a single chain Fv fragment (scFv), single domain antibody, or a camelid antibody.
   427. The AAV particle of any one of embodiments 422-426, wherein the encoded antibody comprises a heavy chain constant region, encoded by a nucleotide sequence having at least 90% (e.g., at least about 95, 96, 97, 98, or 99%) sequence identity to the nucleotide sequence of SEQ ID NO: 5219.
   428. The AAV particle of any one of embodiments 422-427, wherein the encoded antibody molecule comprises a light chain constant region encoded by a nucleotide sequence having at least 90% (e.g., at least about 95, 96, 97, 98, or 99%) sequence identity to the nucleotide sequence of SEQ ID NO: 5221.
   429. The AAV particle of any one of embodiments 422-428, wherein the encoded antibody molecule comprises a heavy chain encoded by a nucleotide sequence having at least 90% (e.g., at least about 95, 96, 97, 98, or 99%) sequence identity to the nucleotide sequence of SEQ ID NO: 5271, 5244, or 7590.
   430. The AAV particle of embodiment 429, wherein the nucleotide sequence encoding the heavy chain comprises the nucleotide sequence of SEQ ID NO: 5271, 5244, or 7590.
   431. The AAV particle of any one of embodiments 1-421, wherein the encoded antibody molecule that binds to HER2/ncu, comprises:
(i) a nucleotide sequence encoding a heavy chain comprising a nucleotide sequence with at least 90% (e.g., at least about 95, 96, 97, 98, or 99%) sequence identity to the nucleotide sequence of SEQ ID NO: 5271, 5244, or 7590; and
(ii) a nucleotide sequence encoding a light chain comprising a nucleotide sequence with at least 90% (e.g., at least about 95, 96, 97, 98, or 99%) sequence identity to the nucleotide sequence of SEQ ID NO: 5275 or 5246.
   432. The AAV particle of embodiment 431, wherein the nucleotide sequence encoding the heavy chain comprises the nucleotide sequence of SEQ ID NO: 5271, 5244, or 7590, and the nucleotide sequence encoding the light chain comprises the nucleotide sequence of SEQ ID NO: 5275 or 5246.
   433. The AAV particle of any one of embodiments 1-421, wherein the encoded antibody molecule that binds to HER2/neu, comprises:
(i) a nucleotide sequence encoding a heavy chain variable region (VH) comprising a nucleotide sequence with at least 90% (e.g., at least about 95, 96, 97, 98, or 99%) sequence identity to the nucleotide sequence of SEQ ID NO: 5109; and
(ii) a nucleotide sequence encoding a light chain variable region (VL) comprising a nucleotide sequence with at least 90% (e.g., at least about 95, 96, 97, 98, or 99%) sequence identity to the nucleotide sequence of SEQ ID NO: 5113.
   434. The AAV particle of embodiment 433, wherein the nucleotide sequence encoding VH comprises the nucleotide sequence of SEQ ID NO: 5109.
   435. The AAV particle of embodiments 433 or 434, wherein the nucleotide sequence encoding the VL comprises the nucleotide sequence of SEQ ID NO: 5113.
   436. The AAV particle of any one of embodiments 433-435, wherein the encoded antibody molecule is a full-length antibody, a bispecific antibody, a Fab, a F(ab′)2, a Fv, a single chain Fv fragment (scFv), single domain antibody, or a camelid antibody.
   437. The AAV particle of any one of embodiments 433-435, wherein the encoded antibody molecule comprises a heavy chain constant region encoded by a nucleotide sequence having at least 90% (e.g., at least about 95, 96, 97, 98, or 99%) sequence identity to the nucleotide sequence of SEQ ID NO: 5017.
   438. The AAV particle of any one embodiments 433-437, wherein the encoded antibody molecule comprises a light chain constant region, encoded by a nucleotide sequence having at least 90% (e.g., at least about 95, 96, 97, 98, or 99%) sequence identity to the nucleotide sequence of SEQ ID NO: 5007.
   439. The AAV particle of any one of embodiments 433-438, wherein the encoded antibody molecule comprises a heavy chain encoded by a nucleotide sequence having at least 90% (e.g., at least about 95, 96, 97, 98, or 99%) sequence identity to the nucleotide sequence of SEQ ID NO: 5111.
   440. The AAV particle of embodiment 439, wherein the nucleotide sequence encoding the heavy chain comprises the nucleotide sequence of SEQ ID NO: 5111.
   441. The AAV particle of any one of embodiments 1-421, wherein the encoded antibody molecule that binds to HER2/neu, comprises:
(i) a nucleotide sequence encoding a heavy chain comprising a nucleotide sequence with at least 90% (e.g., at least about 95, 96, 97, 98, or 99%) sequence identity to the nucleotide sequence of SEQ ID NO: 5111; and
(ii) a nucleotide sequence encoding a light chain comprising a nucleotide sequence with at least 90% (e.g., at least about 95, 96, 97, 98, or 99%) sequence identity to the nucleotide sequence of SEQ ID NO: 5115.
   442. The AAV particle of embodiment 441, wherein the nucleotide sequence encoding the heavy chain comprises the nucleotide sequence of SEQ ID NO: 5111, and the nucleotide sequence encoding the light chain comprises the nucleotide sequence of SEQ ID NO: 5115.
   443. The AAV particle of any one of embodiments 1-421, wherein the encoded antibody molecule that binds to HER2/neu comprises:
(i) a nucleotide sequence encoding a heavy chain variable region (VH) comprising a nucleotide sequence with at least 90% (e.g., at least about 95, 96, 97, 98, or 99%) sequence identity to the nucleotide sequence of SEQ ID NO: 5002; and
(ii) a nucleotide sequence encoding a light chain variable region (VL) comprising a nucleotide sequence with at least 90% (e.g., at least about 95, 96, 97, 98, or 99%) sequence identity to the nucleotide sequence of SEQ ID NO: 5005.
   444. The AAV particle of embodiment 443, wherein the nucleotide sequence encoding the VH comprises the nucleotide sequence of SEQ ID NO: 5002.
   445. The AAV particle of embodiments 443 or 444, wherein the nucleotide sequence encoding the VL comprises the nucleotide sequence of SEQ ID NO: 5005.
   446. The AAV particle of any one of embodiments 443-445, wherein the encoded antibody molecule is a full-length antibody, a bispecific antibody, a Fab, a F(ab′)2, a Fv, a single chain Fv fragment (scFv), single domain antibody, or a camelid antibody.
   447. The AAV particle of embodiment 446, wherein the antibody molecule is a scFv and the nucleotide sequence encoding the scFv comprises the nucleotide sequence of SEQ ID NO: 5352, or a nucleotide sequence with at least 90% (e.g., at least about 95, 96, 97, 98, or 99%) sequence identity thereto.
   448. The AAV particle of any one embodiments 443-446, wherein the encoded antibody molecule comprises a heavy chain constant region encoded by a nucleotide sequence having at least 80% (e.g., at least about 85, 90, 95, 96, 97, 98, or 99%) sequence identity to the nucleotide sequence of SEQ ID NO: 5003.
   449. The AAV particle of any one of embodiments 443-446, wherein the encoded antibody molecule comprises a light chain constant region encoded by a nucleotide sequence having at least 80% (e.g., at least about 85, 90, 95, 96, 97, 98, or 99%) sequence identity to the nucleotide sequence of SEQ ID NO: 5007.
   450. The AAV particle of any of the preceding embodiments, wherein the encoded antibody molecule comprises a heavy chain encoded by a nucleotide sequence having at least 90% (e.g., at least about 95, 96, 97, 98, or 99%) sequence identity to the nucleotide sequence of SEQ ID NO: 5033 or 5369.
   451. The AAV particle of embodiment 450, wherein the nucleotide sequence encoding the heavy chain comprises the nucleotide sequence of SEQ ID NO: or 5369.
   452. The AAV particle of any one of embodiments 1-421, wherein the antibody molecule that binds to HER2/neu, comprises:
(i) a nucleotide sequence encoding a heavy chain comprising a nucleotide sequence with at least 90% (e.g., at least about 95, 96, 97, 98, or 99%) sequence identity to the nucleotide sequence of SEQ ID NO: 5033; and
(ii) a nucleotide sequence encoding a light chain comprising a nucleotide sequence with at least 90% (e.g., at least about 95, 96, 97, 98, or 99%) sequence identity to the nucleotide sequence of SEQ ID NO: 5035.
   453. The AAV particle of embodiment 452, wherein the nucleotide sequence encoding the heavy chain comprises the nucleotide sequence of SEQ ID NO: 5033, and the nucleotide sequence encoding the light chain constant region comprises the nucleotide sequence of SEQ ID NO: 5035.
   454. The AAV particle of any one of embodiments 1-421, wherein the antibody molecule that binds to HER2/neu, comprises:
(i) a nucleotide sequence encoding a heavy chain variable region (VH) comprising a nucleotide sequence with at least 90% (e.g., at least about 95, 96, 97, 98, or 99%) sequence identity to the nucleotide sequence of SEQ ID NO: 5261; and
(ii) a nucleotide sequence encoding a light chain variable region (VL) comprising a nucleotide sequence with at least 90% (e.g., at least about 95, 96, 97, 98, or 99%) sequence identity to the nucleotide sequence of SEQ ID NO: 5266.
   455. The isolated nucleic acid of embodiment 454, wherein the nucleotide sequence encoding the VH is comprises the nucleotide sequence of SEQ ID NO: 5261.
   456. The isolated nucleic acid of embodiments 454 or 455, wherein the nucleotide sequence encoding the VL comprises the nucleotide sequence of SEQ ID NO: 5266.
   457. The isolated nucleic acid of any one of embodiments 454-456, wherein the encoded antibody molecule is a full-length antibody, a bispecific antibody, a Fab, a F(ab′)2, a Fv, a single chain Fv fragment (scFv), single domain antibody, or a camelid antibody.
   458. The AAV particle of any one of embodiments 454-457, wherein the encoded antibody molecule comprises a nucleotide sequence encoding a heavy chain constant region comprises a nucleotide sequence with at least 90% (e.g., at least about 95, 96, 97, 98, or 99%) sequence identity to the nucleotide sequence of SEQ ID NO: 5215.
   459. The AAV particle of any one of embodiments 454-458, wherein the encoded antibody molecule comprises a nucleotide sequence encoding a light chain constant region comprising a nucleotide sequence with at least 90% (e.g., at least about 95, 96, 97, 98, or 99%) sequence identity to the nucleotide sequence of SEQ ID NO: 5217.
   460. The AAV particle of any one of embodiments 454-459, wherein the encoded antibody molecule comprises a nucleotide sequence encoding a heavy chain comprising a nucleotide sequence with at least 90% (e.g., at least about 95, 96, 97, 98, or 99%) sequence identity to the nucleotide sequence of SEQ ID NO: 5263.
   461. The isolated nucleic acid of embodiment 460, wherein the nucleotide sequence encoding the heavy chain comprises the nucleotide sequence of SEQ ID NO: 5263.
   462. The AAV particle of any one of embodiments 1-421, wherein the encoded antibody molecule that binds to HER2/neu, comprises:
(i) a nucleotide sequence encoding a heavy chain comprising a nucleotide sequence with at least 90% (e.g., at least about 95, 96, 97, 98, or 99%) sequence identity to the nucleotide sequence of SEQ ID NO: 5263; and
(ii) a nucleotide sequence encoding a light chain comprising a nucleotide sequence with at least 90% (e.g., at least about 95, 96, 97, 98, or 99%) sequence identity to the nucleotide sequence of SEQ ID NO: 5268.
   463. The AAV particle of embodiment 462, wherein the nucleotide sequence encoding the heavy chain comprises the nucleotide sequence of SEQ ID NO: 5263, and the nucleotide sequence encoding the light chain comprises the nucleotide sequence of SEQ ID NO: 5268.
   464. The AAV particle of any one of embodiments 1-421, wherein the encoded antibody molecule that binds to HER2/neu, comprises:
(i) a nucleotide sequence encoding a heavy chain comprising a nucleotide sequence with at least 90% (e.g., at least about 95, 96, 97, 98, or 99%) sequence identity to the nucleotide sequence of SEQ ID NO: 5173, and a nucleotide sequence encoding a light chain comprising a nucleotide sequence with at least 90% (e.g., at least about 95, 96, 97, 98, or 99%) sequence identity to the nucleotide sequence of SEQ ID NO: 5177;
(ii) a nucleotide sequence encoding a heavy chain comprising a nucleotide sequence with at least 90% (e.g., at least about 95, 96, 97, 98, or 99%) sequence identity to the nucleotide sequence of SEQ ID NO: 5049, and a nucleotide sequence encoding a light chain comprising a nucleotide sequence with at least 90% (e.g., at least about 95, 96, 97, 98, or 99%) sequence identity to the nucleotide sequence of SEQ ID NO: 5051;
(iii) a nucleotide sequence encoding a heavy chain comprising a nucleotide sequence with at least 90% (e.g., at least about 95, 96, 97, 98, or 99%) sequence identity to the nucleotide sequence of SEQ ID NO: 5065, and a nucleotide sequence encoding a light chain comprising a nucleotide sequence with at least 90% (e.g., at least about 95, 96, 97, 98, or 99%) sequence identity to the nucleotide sequence of SEQ ID NO: 5067;
(iv) a nucleotide sequence encoding a heavy chain comprising a nucleotide sequence with at least 90% (e.g., at least about 95, 96, 97, 98, or 99%) sequence identity to the nucleotide sequence of SEQ ID NO: 5083, and a nucleotide sequence encoding a light chain comprising a nucleotide sequence with at least 90% (e.g., at least about 95, 96, 97, 98, or 99%) sequence identity to the nucleotide sequence of SEQ ID NO: 5087;
(v) a nucleotide sequence encoding a heavy chain comprising a nucleotide sequence with at least 90% (e.g., at least about 95, 96, 97, 98, or 99%) sequence identity to the nucleotide sequence of SEQ ID NO: 5193, and a nucleotide sequence encoding a light chain comprising a nucleotide sequence with at least 90% (e.g., at least about 95, 96, 97, 98, or 99%) sequence identity to the nucleotide sequence of SEQ ID NO: 5197;
(vi) a nucleotide sequence encoding a heavy chain comprising a nucleotide sequence with at least 90% (e.g., at least about 95, 96, 97, 98, or 99%) sequence identity to the nucleotide sequence of SEQ ID NO: 5225, and a nucleotide sequence encoding a light chain comprising a nucleotide sequence with at least 90% (e.g., at least about 95, 96, 97, 98, or 99%) sequence identity to the nucleotide sequence of SEQ ID NO: 5229;
(vii) a nucleotide sequence encoding a heavy chain comprising a nucleotide sequence with at least 90% (e.g., at least about 95, 96, 97, 98, or 99%) sequence identity to the nucleotide sequence of SEQ ID NO: 5091, and a nucleotide sequence encoding a light chain comprising a nucleotide sequence with at least 90% (e.g., at least about 95, 96, 97, 98, or 99%) sequence identity to the nucleotide sequence of SEQ ID NO: 5095; or
(viii) a nucleotide sequence encoding a heavy chain comprising a nucleotide sequence with at least 90% (e.g., at least about 95, 96, 97, 98, or 99%) sequence identity to the nucleotide sequence of SEQ ID NO: 5131, and a nucleotide sequence encoding a light chain comprising a nucleotide sequence with at least 90% (e.g., at least about 95, 96, 97, 98, or 99%) sequence identity to the nucleotide sequence of SEQ ID NO: 5135.
   465. The AAV particle of embodiment 464, wherein:
(i) the nucleotide sequence encoding the heavy chain comprises the nucleotide sequence of SEQ ID NO: 5173, and the nucleotide sequence encoding the light chain comprises the nucleotide sequence of SEQ ID NO: 5177;
(ii) the nucleotide sequence encoding the heavy chain comprises the nucleotide sequence of SEQ ID NO: 5049, and the nucleotide sequence encoding the light chain comprises the nucleotide sequence of SEQ ID NO: 5051;
(iii) the nucleotide sequence encoding the heavy chain comprises the nucleotide sequence of SEQ ID NO: 5065, and the nucleotide sequence encoding the light chain comprises the nucleotide sequence of SEQ ID NO: 5067;
(iv) the nucleotide sequence encoding the heavy chain comprises the nucleotide sequence of SEQ ID NO: 5083, and the nucleotide sequence encoding the light chain comprises the nucleotide sequence of SEQ ID NO: 5087;
(v) the nucleotide sequence encoding the heavy chain comprises the nucleotide sequence of SEQ ID NO: 5193, and the nucleotide sequence encoding the light chain comprises the nucleotide sequence of SEQ ID NO: 5197;
(vi) the nucleotide sequence encoding the heavy chain comprises the nucleotide sequence of SEQ ID NO: 5225, and the nucleotide sequence encoding the light chain comprises the nucleotide sequence of SEQ ID NO: 5229;
(vii) the nucleotide sequence encoding the heavy chain comprises the nucleotide sequence of SEQ ID NO: 5091, and the nucleotide sequence encoding the light chain comprises the nucleotide sequence of SEQ ID NO: 5095; or
(viii) the nucleotide sequence encoding the heavy chain comprises the nucleotide sequence of SEQ ID NO: 5131, and the nucleotide sequence encoding the light chain comprises the nucleotide sequence of SEQ ID NO: 5135.
   466. The AAV particle of any one of the embodiments 404-426, 433-436, 443-446 or 454-457 wherein the encoded antibody comprises an Fc region or functional variant thereof.
   467. The AAV particle of any one of embodiments 404-426, 434-436, 443-446 or 454-457 wherein the encoded antibody comprises an scFv and an Fc region.
   468. The AAV particle of embodiment 466 or 467, wherein the Fc region has reduced affinity, e.g., ablated, affinity for an Fc receptor, e.g., as compared to a reference, wherein the reference is a wild-type Fc receptor.
   469. The AAV particle of any one of embodiments 466-468, wherein the Fc region comprises a mutation at one, two, or all of positions I253 (e.g., I253A), H310 (e.g., H310A or H310Q), and/or H435 (e.g., H435A or H435Q), according to EU numbering.
   470. The AAV particle of any one of embodiments 466-469, wherein the Fc region comprises a mutation at positions I253 (e.g., I253A), H310 (e.g., H310A or H310Q), and H435 (e.g., H435A or H435Q), according to EU numbering.
   471. The AAV particle of any one of embodiments 466-469, wherein the Fc region comprises one, two, or all of the mutations I253A, H310A, and/or H435A, according to EU numbering.
   472. The AAV particle of any one of embodiments 466-469, or 471, wherein the Fc region comprises one, two, or all of A at position 253, A at position 310, and/or A at position 435, according to EU numbering.
   473. The AAV particle of any one of embodiments 466-472, wherein the Fc region comprises the mutations I253A, H310A, and H435A, according to EU numbering.
   474. The AAV particle of any one of embodiments 466-473, wherein the Fc region comprises A at position 253, A at position 310, and A at position 435, according to EU numbering.
   475. The AAV particle of embodiment 466 or 467, wherein the Fc region has reduced effector function (e.g., reduced ADCC), compared to a reference wherein the reference is a wild-type Fc receptor.
   476. The AAV particle of any one of embodiments 466-474, wherein the Fc region comprises a mutation at one, two, three, four, or all of positions L235 (e.g., L235V), F243 (e.g., F243L), R292 (e.g., R292P), Y300 (e.g., Y300L), and/or P396 (e.g., P396L), numbered according to the EU index as in Kabat.
   477. The AAV particle of any one of embodiments 466-476, wherein the Fc region comprises a mutation at positions L235 (e.g., L235V), F243 (e.g., F243L), R292 (e.g., R292P), Y300 (e.g., Y300L), and P396 (e.g., P396L), numbered according to the EU index as in Kabat.
   478. The AAV particle of any one of embodiments 466-477, wherein the Fc region comprises one, two, three, four, or all of the mutations L235V, F243L, R292P, Y300L, and/or P396L, numbered according to the EU index as in Kabat.
   479. The AAV particle of any one of embodiments 466-478, wherein the Fc region comprises one, two, three, four, or all of V at position 235, L at position 243, P at position 292, L at position 300, and/or L at position 396, numbered according to the EU index as in Kabat.
   480. The AAV particle of any one of embodiments 466-479, wherein the Fc region comprises the mutations L235V, F243L, R292P, Y300L, and P396L, numbered according to the EU index as in Kabat.
   481. The AAV particle of any one of embodiments 466-480, wherein the Fc region comprises V at position 235, L at position 243, P at position 292, L at position 300, and L at position 396, numbered according to the EU index as in Kabat.
   482. The AAV particle of any one of embodiments 466-481, wherein the Fc region comprises a mutation at L235 (e.g., L235V), F243 (e.g., F243L), R292 (e.g., R292P), Y300 (e.g., Y300L), P396 (e.g., P396L), I253 (e.g., I253A), H310 (e.g., H310A or H310Q), and H435 (e.g., H435A or H435Q), numbered according to the EU index as in Kabat.
   483. The AAV particle of any one of embodiments 466-482, wherein the Fc region comprises the mutations L235V, F243L, R292P, Y300L, P396L, I253A, H310A, and H435A, numbered according to the EU index as in Kabat.
   484. The AAV particle of any one of embodiments 466-474 or 476-483, wherein the Fc region comprises V at position 235, L at position 243, P at position 292, L at position 300, L at position 396, A at position 253, A at position 310, and A at position 435, numbered according to the EU index as in Kabat.
   485. The AAV particle of embodiment 466 or 467, wherein
(i) the encoded Fc region comprises the amino acid sequence of SEQ ID NO: 5278, or an amino acid sequence with at least 80% (e.g., at least about 85, 90, 95, 96, 97, 98, or 99%) sequence identity thereto; and/or
(ii) the nucleotide sequence encoding the Fc region comprises the nucleotide sequence of SEQ ID NO: 5277, or a nucleotide sequence with at least 80% (e.g., at least about 85, 90, 95, 96, 97, 98, or 99%) sequence identity thereto.
   486. The AAV particle of embodiment 467, wherein the nucleotide sequence encoding the scFv comprises the nucleotide sequence of SEQ ID NO: 5352, or a nucleotide sequence with at least 90% (e.g., at least 95, 96, 97, 98, or 99%) sequence identity thereto, and the nucleotide sequence encoding the Fe region comprises the nucleotide sequence of SEQ ID NO: 5277, or a nucleotide sequence with at least 80% (e.g., at least about 85, 90, 95, 96, 97, 98, or 99%) sequence identity thereto.
   487. The AAV particle of any one of the preceding embodiments, wherein the transgene further encodes an antibody mimetic, e.g., a designed ankyrin repeat protein (DARPIN), optionally wherein the encoded DARPIN comprises the amino acid sequence of SEQ ID NO: 5370, or an amino acid sequence with at least 80% (e.g., 85, 90, 95, 96, 97, 98, or 99%) sequence identity thereto, or the nucleotide sequence encoding the DARPIN comprises the nucleotide sequence of SEQ ID NO: 5371, or a nucleotide sequence with at least 80% (e.g., 85, 90, 95, 96, 97, 98, or 99%) sequence identity thereto.
   488. The AAV particle of any one of the preceding embodiments, wherein the transgene further encodes a fynomer, optionally wherein the encoded fynomer comprises the amino acid sequence of SEQ ID NO: 5156, or an amino acid sequence with at least 80% (e.g., 85, 90, 95, 96, 97, 98, or 99%) sequence identity thereto, or the nucleotide sequence encoding the fynomer comprises the nucleotide sequence of SEQ ID NO: 5155, or a nucleotide sequence with at least 80% (e.g., 85, 90, 95, 96, 97, 98, or 99%) sequence identity thereto.
   489. The AAV particle of any one of the preceding embodiments, further encoding a signal sequence, optionally wherein the signal sequence comprises a nucleotide sequence of any of the signal sequences listed in Table 11A, or a nucleotide sequence with at least 95% sequence identity thereto.
   490. The AAV particle of embodiment 489, further encoding a second signal sequence, optionally wherein the second signal sequence comprises a nucleotide sequence of any of the signal sequences listed in Table 11A, or a nucleotide sequence with at least 95% sequence identity thereto.
   491. The AAV particle of embodiment 490 wherein:
(i) the nucleotide sequence encoding the signal sequence comprises the nucleotide sequence of SEQ ID NO: 5157, and is located 5′ relative to the nucleotide sequence encoding the VH and/or the heavy chain;
(ii) the nucleotide sequence encoding the signal sequence comprises the nucleotide sequence of SEQ ID NO: 5032, and is located 5′ relative to the nucleotide sequence encoding the VH and/or the heavy chain; and/or
(iii) the nucleotide sequence encoding the signal sequence comprises the nucleotide sequence of SEQ ID NO: 5159, and is located 5′ relative to the nucleotide sequence encoding the VL and/or the light chain.
   492. The AAV particle of any one of the preceding embodiments, wherein:
(i) the sequences of the encoded VH and VL are connected directly, e.g., without a linker;
(ii) the sequences of the encoded VH and VL are connected via a linker;
(iii) the sequences of the encoded heavy chain and light chain are connected directly. e.g., without a linker; or
(iv) the sequences of the encoded heavy chain and light chain are connected via a linker.
   493. The AAV particle of embodiment 492, wherein:
(i) the linker comprises a nucleotide sequence of any of the linker sequences listed in Table 41, or a nucleotide sequence with at least 95% sequence identity thereto;
(ii) the linker comprises an encoded furin cleavage site;
(iii) the linker comprises an encoded T2A linker; and/or
(iv) the linker comprises a glycine-serine linker, e.g., a G4S linker (SEQ ID NO: 4535) or a (G4S)3 linker (SEQ ID NO: 4537).
   494. The AAV particle embodiment 492 or 493, wherein:
(i) the linker comprises an encoded furin cleavage site and/or comprises the nucleotide sequence of SEQ ID NO: 1724 or 6557; and/or
(ii) the linker comprises an encoded T2A linker and/or the nucleotide sequence of SEQ ID NO: 1726 or 6558; and/or
(iii) the linker comprises an encoded glycine-serine linker encoded by the nucleotide sequence of SEQ ID NOs: 6597, 5161, 5162, or 5347, or the nucleotide sequence of GGCTCT.
   495. The AAV particle of any one of the preceding embodiments, wherein the transgene encodes a second antigen-binding region having a different binding specificity than the antigen-binding region that binds to HER2/neu.
   496. The AAV particle of embodiment 495, wherein the second antigen-binding region binds to a molecule selected from the group consisting of a cancer- or tumor-associated antigen; a cancer-associated integrin; a T cell and/or NK cell antigen; an angiogenic factor or other cancer-associated growth factor; receptor for an angiogenic factor; and a receptor associated with cancer progression.
   497. The AAV particle of embodiment 495 or 496, wherein the second antigen-binding region binds to carcinoembryonic antigen (CEA), prostate specific antigen (PSA), RAGE (renal antigen), α-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), ganglioside antigens, tyrosinase, gp75, c-Met, C-myc, Mart1., MelanA, MUM-1, MUM-2, MUM-3, HLA-B7, Ep-CAM or a cancer-associated integrin, such as a5133 integrin, a T cell and/or NK cell antigen, such as CD3 or CD16, an angiogenic factor or other cancer-associated growth factor, such as a vascular endothelial growth factor, a fibroblast growth factor, epidermal growth factor, and receptors associated with cancer progression.
   498. The AAV particle of embodiment 495 or 496, wherein the second antigen-binding region binds HER1, HER3, or HER4.
   499. The AAV particle of any one of embodiments 495, 496 or 498, wherein the second antigen-binding site binds a different, preferably non-blocking, site on HER2.
   500. The AAV particle of embodiment 495, wherein the first heavy chain variable region is encoded by the nucleotide sequence of SEQ ID NO: 5253, 5257 or 5261, and the second heavy chain variable is encoded by the nucleotide sequence of SEQ ID NO: 5254, 5258 or 5262.
   501. The AAV particle of any one of embodiments 495-500, wherein the first light chain variable region is encoded by the nucleotide sequence of SEQ ID NO: 5255, 5259 or 5265, and a second light chain variable region encoded by the nucleotide sequence of SEQ ID NO: 5256, 5260 or 5267.
   502. The AAV particle of any one of embodiments 422-501, wherein the encoded antibody is a multispecific antibody molecule, e.g., a bispecific antibody molecule.
   503. The AAV particle of any one of embodiments 422-502, wherein the encoded antibody is a bispecific, e.g., biparatopic, antibody molecule.
   504. The AAV particle of embodiment 503, wherein the encoded bispecific, e.g., biparatopic, antibody molecule comprises at least two antigen binding domains for two different domains of HER2.
   505. The AAV particle of embodiment 503 or 504, wherein the encoded bispecific, e.g., biparatopic, antibody molecule comprises a first antigen binding domain that binds domain IV of HER2 and a second antigen binding domain that binds domain I of HER2.
   506. The AAV particle of embodiment 503 or 504, wherein the encoded bispecific, e.g., biparatopic, antibody molecule comprises a first antigen binding domain that binds domain I of HER2 and a second antigen binding domain that binds domain IV of HER2.
   507. The AAV particle of any one of embodiments 503-506, wherein the first and/or second antigen binding domain comprise an IgG antibody, single-chain Fv (scFv), a scFv fragment, a Fab, a single-chain Fab (scFabs), a single-chain antibody, a diabody, an antibody variable domain, a VHH, a single domain antibody, and/or a nanobody.
   508. The AAV particle of any one of embodiments 503-507, wherein:
(i) the first antigen binding domain comprises an scFv, and the second antigen binding domain comprises a full antibody, e.g., an IgG antibody;
(ii) the first antigen binding domain comprises an antibody mimetic, e.g., a DARPIN, and the second antigen binding domain comprises a full antibody, e.g., an IgG antibody; or
(iii) the first antigen binding domain comprises a Fyn SH3-derived binding polypeptide (e.g., a fynomer), and the second antigen binding domain comprises a full antibody, e.g., an IgG antibody.
   509. The AAV particle of any one of embodiment 503-508, wherein:
(i) the first antigen binding domain comprises a full antibody, e.g., an IgG antibody, and the second antigen binding domain comprises an scFv;
(ii) the first antigen binding domain comprises a full antibody, e.g., an IgG antibody, and the second antigen binding domain comprises an antibody mimetic, e.g., a DARPIN; or
(iii) the first antigen binding domain comprises a full antibody, e.g., an IgG antibody, and the second antigen binding domain comprises a Fyn SH3-derived binding polypeptide (e.g., a fynomer).
   510. The AAV particle of any one of embodiments 503-508, wherein:
(i) the first antigen binding domain that binds domain IV of HER2, e.g., an scFv that binds domain IV of HER2, is situated N-terminal of the second antigen binding domain that binds domain I of HER2, e.g., a full antibody, e.g., an IgG antibody that binds domain I of HER2;
(ii) the first antigen binding domain that binds domain I of HER2, e.g., an antibody mimetic, e.g., a DARPIN, is situated N-terminal of the second antigen binding domain that binds domain IV of HER2, e.g., a full antibody, e.g., an IgG antibody that binds domain IV of HER2; or
(iii) the first antigen binding domain that binds domain I of HER2, e.g., a Fyn SH3-derived binding polypeptide (e.g., a fynomer), is situated N-terminal of the second antigen binding domain that binds domain IV of HER2, e.g., a full antibody, e.g., an IgG antibody that binds domain IV of HER2.
   511. The AAV particle of any one of embodiments, 503-510, wherein the encoded bispecific antibody molecule comprises:
(i) a first polypeptide comprising, from N-terminal to C-terminal: VH of the first binding domain, first peptide linker (e.g., a (G4S)3 linker (SEQ ID NO: 4537)), VL of first binding domain, second peptide linker (e.g., a (G4S) linker (SEQ ID NO: 4535)), VL of the second binding domain and CL; and
(ii) a second polypeptide comprising, from N-terminal to C-terminal: VH of the second binding domain, CH1, CH2, and CH3.
   512. The AAV particle of any one of embodiments, 503-510, wherein the encoded bispecific antibody molecule comprises:
(i) a first polypeptide comprising, from N-terminal to C-terminal: a DARPIN, a peptide linker (e.g., a (G4S)3 linker (SEQ ID NO: 4537)), VL of the second binding domain and CL; and
(ii) a second polypeptide comprising from N-terminal to C-terminal: VH of the second binding domain, CH1, CH2, and CH3.
   513. The AAV particle of any one of embodiments, 503-510, wherein the encoded bispecific antibody molecule comprises:
(i) a first polypeptide comprising, from N-terminal to C-terminal: a Fyn SH3-derived binding polypeptide, a peptide linker (e.g., a (G4S)3 linker (SEQ ID NO: 4537)), VL of the second binding domain and CL; and
(ii) a second polypeptide comprising from N-terminal to C-terminal: VH of the second binding domain, CH1, CH2, and CH3.
   514. The AAV particle of any one of embodiments 503-513, which comprises an Fc region, optionally wherein the Fc region, is mutated to have reduced binding to Fc receptor or reduced ADCC, e.g., an Fc region having the mutations L235V, F243L, R292P, Y300L, and P396L, numbered according to the EU index as in Kabat.
   515. The AAV particle of any one of embodiments 1-422, wherein the encoded antibody molecule is a multispecific, e.g., bispecific, antibody molecule comprising at least two antigen binding domains for two different domains of HER2, optionally wherein the first antigen binding domain binds to domain I of HER2, and the second antigen binding domain binds to domain IV of HER2.
   516. The AAV particle of embodiment 515, wherein:
(i) the first antigen binding domain binds domain I of HER2 and the second antigen binding domain that binds domain IV of HER2;
(ii) the first antigen binding domain that binds domain II of HER2 and the second antigen binding domain that binds domain IV of HER2;
(iii) the first antigen binding domain that binds domain III of HER2 and the second antigen binding domain that binds domain IV of HER2;
(iv) the first antigen binding domain that binds domain I of HER2 and the second antigen binding domain that binds domain II of HER2;
(v) the first antigen binding domain that binds domain I of HER2 and the second antigen binding domain that binds domain III of HER2; or
(vi) the first antigen binding domain that binds domain II of HER2 and the second antigen binding domain that binds domain III of HER2.
   517. The AAV particle of embodiment 515 or 516, wherein the first antigen binding domain binds domain I of HER2 and the second antigen binding domain that binds domain IV of HER2.
   518. The AAV particle of embodiment 515 or 516, wherein the first antigen binding domain binds domain IV of HER2 and the second antigen binding domain that binds domain I of HER2.
   519. The AAV particle of any one of embodiments 515-518, wherein the first antigen binding domain and/or the second antigen binding domain comprise:
(i) a heavy chain variable region comprising one, two, or three HC CDR sequence of any of the CDR sequences of Table 3A-3C; and/or
(ii) a light chain variable region comprising one, two, or three LC CDR sequence of any of the CDR sequences of Table 3A-3C.
   520. The AAV particle of any one of embodiments 515-519, wherein the first antigen binding domain and/or the second antigen binding domain comprise:
(i) a heavy chain variable region (VH) comprising an amino acid sequence of any of the VH sequences of Table 3A-3C, or a sequence having at least 80% (e.g., 85, 90, 95, 96, 97, 98, or 99%) sequence identity to any of the VH sequences of Table 3A-3C; and/or
(ii) a light chain variable region (VL) comprising an amino acid sequence of any of the VL sequences of Table 3A-3C, or a sequence having at least 80% (e.g., 85, 90, 95, 96, 97, 98, or 99%) sequence identity to any of the VL sequences of Table 3A-3C.
   521. The AAV particle of any one of embodiments 515-519, wherein the first antigen binding domain and/or the second antigen binding domain comprise:
(i) a heavy chain constant region comprising an amino acid sequence of any of the heavy chain constant region sequences of Table 3A-3C, or a sequence having at least 80% (e.g., 85, 90, 95, 96, 97, 98, or 99%) sequence identity to the heavy chain constant region sequences of Table 3A-3C; and/or
(ii) a light chain constant region (CL) comprising an amino acid sequence of any of the CL sequences of Table 3A-3C, or a sequence having at least 80% (e.g., 85, 90, 95, 96, 97, 98, or 99%) sequence identity to any of the CL sequences of Table 3A-3C.
   522. The AAV particle of any one of embodiments 515-521, wherein the first antigen binding domain and/or the second antigen binding domain comprise:
(i) a heavy chain comprising an amino acid sequence of any of the heavy chain sequences of Table 3A-3C, or a sequence having at least 80% (e.g., 85, 90, 95, 96, 97, 98, or 99%) sequence identity to any of the heavy chain sequences of Table 3A-3C; and/or
(ii) a light chain comprising an amino acid sequence of any of the light chain sequences of Table 3A-3C, or a sequence having at least 80% (e.g., 85, 90, 95, 96, 97, 98, or 99%) sequence identity to any of the light chain sequences of Table 3A-3C.
   523. The AAV particle of any one of embodiments 515-522, wherein the first antigen binding domain and/or the second antigen binding domain comprises a heavy chain variable region or a heavy chain comprising at least 1-4, e.g., at least one, two, three, or four, modifications, e.g., substitutions, in a HC CDR3 region, e.g., an HC CDR3 according to SEQ ID NO: 5001. 524. The AAV particle of any one of embodiments 515-523, wherein the first antigen binding domain and/or the second antigen binding domain comprises a heavy chain variable region or a heavy chain comprising:
(i) an amino acid substitution at one, two, three, or all of positions 102 (e.g., D102W), 107 (e.g., M107F), 108 (e.g., D108A), and/or 109 (e.g., Y109L), numbered according to the amino acid sequence of SEQ ID NO: 5001;
(ii) one, two, three, or all of an amino acid other than D at position 102, an amino acid other than M at position 107, an amino acid other than D at position 108, and/or an amino acid other than Y at position 109, numbered according to the amino acid sequence of SEQ ID NO: 5001; or
(iii) one, two, three or all of the amino acid W at position 102, the amino acid F at position 107, A at position 108, and/or L at position 109, numbered according to the amino acid sequence of SEQ ID NO: 5001.
   525. The AAV particle of any one of embodiments 515-524, wherein the first antigen binding domain and/or the second antigen binding domain comprises a heavy chain variable region or a heavy chain comprising:
(a) the amino acid W at position 102, the amino acid F at position 107, the amino acid A position 108, and the amino acid L at position 109, relative to a reference sequence numbered according to the amino acid sequence of SEQ ID NO: 5001;
(b) the amino acid W at position 102, the amino acid F at position 107, and the amino acid A position 108, relative to a reference sequence numbered according to the amino acid sequence of SEQ ID NO: 5001;
(c) the amino acid W at position 102, the amino acid F at position 107, and the amino acid L at position 109, relative to a reference sequence numbered according to the amino acid sequence of SEQ ID NO: 5001;
(d) the amino acid W at position 102, the amino acid A position 108, and the amino acid L at position 109, relative to a reference sequence numbered according to the amino acid sequence of SEQ ID NO: 5001;
(e) the amino acid F at position 107, the amino acid A position 108, and the amino acid L at position 109, relative to a reference sequence numbered according to the amino acid sequence of SEQ ID NO: 5001.
   526. The AAV particle of any one of embodiments 515-525, wherein the first antigen binding domain and/or the second antigen binding domain comprises a heavy chain variable region or a heavy chain comprising:
(i) the amino acid substitutions D98W, M100F, D101A, and Y102L, numbered according to Kabat;
(ii) the amino acid substitutions D98W, M100F, and D101A, numbered according to Kabat;
(iii) the amino acid substitutions D98W, M100F, and Y102L, numbered according to Kabat;
(iv) the amino acid substitutions D98W, D101A, and Y102L, numbered according to Kabat;
(v) the amino acid substitutions M100F, D101A, and Y102L, numbered according to Kabat.
   527. The AAV particle of any one of embodiments 515-526, wherein the first antigen binding domain and/or the second antigen binding domain comprises a heavy chain variable region or a heavy chain comprising an HC CDR1, a HC CDR2, a HC CDR3, wherein:
(i) the HC CDR1, HC CDR2, HC CDR3 comprise the sequences of SEQ ID NO: 5281, 5282, 6510, respectively;
(ii) the HC CDR1, HC CDR2, HC CDR3 comprise the sequences of SEQ ID NO: 5281, 5282, 6515, respectively;
(iii) the HC CDR1, HC CDR2, HC CDR3 comprise the sequences of SEQ ID NO: 5281, 5282, 6520, respectively;
(iv) the HC CDR1, HC CDR2, HC CDR3 comprise the sequences of SEQ ID NO: 5281, 5282, 6525, respectively; or
(v) the HC CDR1, HC CDR2, HC CDR3 comprise the sequences of SEQ ID NO: 5281, 5282, 6530, respectively.
   528. The AAV particle of any one of embodiments 515-527, wherein the first antigen binding domain and/or the second antigen binding domain comprises:
(i) a heavy chain variable region comprising a HC CDR1 amino acid sequence of SEQ ID NO: 5281, a HC CDR2 amino acid sequence of SEQ ID NO: 5282, and an HC CDR3 amino acid sequence of SEQ ID NO: 5283, 6510, 6515, 6520, 6525 or 6530; and
(ii) a light chain variable region comprising an LC CDR1 amino acid sequence of SEQ ID NO: 5287, an LC CDR2 amino acid sequence of SEQ ID NO: 5288, and an LC CDR3 amino acid sequence of SEQ ID NO: 5289.
   529. The AAV particle of any one of embodiments 515-528, wherein the first antigen binding domain and/or the second antigen binding domain comprises an HC CDR1, a HC CDR2, a HC CDR3, a LC CDR1, a LC CDR2 and/or an LC CDR3, wherein:
(i) the HC CDR1, HC CDR2, HC CDR3, LC CDR1, LC CDR2, and LC CDR3 sequences comprise the sequences of SEQ ID NO: 5281, 5282, 6510, 5287, 5288, and 5289, respectively;
(ii) the HC CDR1, HC CDR2, HC CDR3, LC CDR1, LC CDR2, and LC CDR3 sequences comprise the sequences of SEQ ID NO: 5281, 5282, 6515, 5287, 5288, and 5289, respectively;
(iii) the HC CDR1, HC CDR2, HC CDR3, LC CDR1, LC CDR2, and LC CDR3 sequences comprise the sequences of SEQ ID NO: 5281, 5282, 6520, 5287, 5288, and 5289, respectively;
(iv) the HC CDR1, HC CDR2, HC CDR3, LC CDR1, LC CDR2, and LC CDR3 sequences comprise the sequences of SEQ ID NO: 5281, 5282, 6525, 5287, 5288, and 5289, respectively;
(v) the HC CDR1, HC CDR2, HC CDR3, LC CDR1, LC CDR2, and LC CDR3 sequences comprise the sequences of SEQ ID NO: 5281, 5282, 6530, 5287, 5288, and 5289, respectively.
   530. The AAV particle of any one of embodiments 515-529, wherein the first antigen binding domain comprises:
(i) a heavy chain variable region comprising a HC CDR1 amino acid sequence of SEQ ID NO: 5358, a HC CDR2 amino acid sequence of SEQ ID NO: 5359, and an HC CDR3 amino acid sequence of SEQ ID NO: 5360, 6535, 6538, 6541, 6544, or 6547; and
(ii) a light chain variable region comprising an LC CDR1 amino acid sequence of SEQ ID NO: 5355, an LC CDR2 amino acid sequence of SEQ ID NO: 5356, and an LC CDR3 amino acid sequence of SEQ ID NO: 5357.
   531. The AAV particle of any one of embodiments 515-530, wherein the second antigen binding domain comprises:
(i) a HC CDR1 amino acid sequence of SEQ ID NO: 5361, a HC CDR2 amino acid sequence of SEQ ID NO: 5362, and an HC CDR3 amino acid sequence of SEQ ID NO: 5363; and
(ii) a light chain variable region comprising an LC CDR1 amino acid sequence of SEQ ID NO: 5317, an LC CDR2 amino acid sequence of SEQ ID NO: 5318, and an LC CDR3 amino acid sequence of SEQ ID NO: 5319.
   532. The AAV particle of any one of one of embodiments 515-531, wherein the first antigen binding domain and/or the second antigen binding domain comprises a heavy chain variable region comprising an amino acid selected from SEQ ID NO: 5001, 5367, 5172, 5106, 5010, 5069, 5192, 5224, 5090, 5110, 5254, 5258, 5130, 5262, 5270, 5326, 6511, 6516, 6521, 6526, 6531, 6536, 6539, 6542, 6545, or 6548, or a sequence substantially identical (e.g., having at least about 80%, 85%, 90%, 92%, 95%, 97%, 98%, or 99% sequence identity) to any of the aforesaid sequences.
   533. The AAV particle of any one of embodiments 515-532, wherein the encoded multispecific antibody comprises:
(i) a first anti-HER2 VH comprising the amino acid sequence of SEQ ID NO: 5262, and a heavy chain constant region comprising the amino acid sequence of SEQ ID NO: 5220; and
(ii) a second chain, which comprises from the N-terminus to the C-terminus, a second anti-HER2 VH comprising the amino sequence of SEQ ID NO: 5290, 6536, 6539, 6542, 6545, or 6548, a (G4S)3 linker (SEQ ID NO: 4537), a first anti-HER2 VL comprising the amino acid sequence of SEQ ID NO: 5266, a (GS) linker, a second anti-HER2 VL comprising the amino acid sequence of SEQ ID NO: 5354, and a light chain constant region (CL) comprising the amino acid sequence of SEQ ID NO: 5218;
or a sequence substantially identical (e.g., having at least 70%, 75%, 80%, 85%, 90%, 95%, or 99% sequence identity) to the any of the aforesaid sequences.
   534. The AAV particle of any one of embodiments 515-533, wherein the encoded antibody molecule comprises an Fc region.
   535. The AAV particle of embodiment 534, wherein the Fc receptor comprises:
(i) a mutation at one or more of (e.g., all of) positions I253 (e.g., I253A), H310 (e.g., H310A or H310Q), and/or H435 (e.g., H435A or H435Q), numbered according to the EU index as in Kabat; and/or
(ii) a mutation at one or more of (e.g., all of) positions L235 (e.g., L235V), F243 (e.g., F243L), R292 (e.g., R292P), Y300 (e.g., Y300L), and P396 (e.g., P396L), numbered according to the EU index as in Kabat.
   536. The AAV particle of any one of embodiments 515-535, wherein the first and/or second antigen binding domain comprise an IgG antibody, single-chain Fv (scFv), a scFv fragment, a Fab, a single-chain Fab (scFabs), a single-chain antibody, a diabody, an antibody variable domain, a VHH, a single domain antibody, and/or a nanobody.
   537. The AAV particle of any one of embodiments 515-536, wherein:
(i) the first antigen binding domain comprises an scFv, and the second antigen binding domain comprises a full antibody, e.g., an IgG antibody;
(ii) the first antigen binding domain comprises an antibody mimetic, e.g., a DARPIN, and the second antigen binding domain comprises a full antibody, e.g., an IgG antibody; or
(iii) the first antigen binding domain comprises a Fyn SH3-derived binding polypeptide (e.g., a fynomer), and the second antigen binding domain comprises a full antibody, e.g., an IgG antibody

538. The AAV particle of any one of embodiments 515-536, wherein:
(i) the first antigen binding domain comprises a full antibody, e.g., an IgG antibody, and the second antigen binding domain comprises an scFv;
(ii) the first antigen binding domain comprises a full antibody, e.g., an IgG antibody, and the second antigen binding domain comprises an antibody mimetic, e.g., a DARPIN; or
(iii) the first antigen binding domain comprises a full antibody, e.g., an IgG antibody, and the second antigen binding domain comprises a Fyn SH3-derived binding polypeptide (e.g., a fynomer).
   539. The AAV particle of any one of embodiments 515-538, wherein:
(i) the first antigen binding domain that binds domain IV of HER2, e.g., an scFv that binds domain IV of HER2, is situated N-terminal of the second antigen binding domain that binds domain I of HER2, e.g., a full antibody, e.g., an IgG antibody that binds domain I of HER2;
(ii) the first antigen binding domain that binds domain I of HER2. e.g., an antibody mimetic, e.g., a DARPIN, is situated N-terminal of the second antigen binding domain that binds domain IV of HER2, e.g., a full antibody, e.g., an IgG antibody that binds domain IV of HER2; or
(iii) the first antigen binding domain that binds domain I of HER2, e.g., a Fyn SH3-derived binding polypeptide (e.g., a fynomer), is situated N-terminal of the second antigen binding domain that binds domain IV of HER2, e.g., a full antibody, e.g., an IgG antibody that binds domain IV of HER2.
   540. The AAV particle of any one of embodiments 515-539, wherein the encoded bispecific antibody molecule comprises:
(i) a first polypeptide comprising, from N-terminal to C-terminal: VH of the first binding domain, first peptide linker (e.g., a (G4S)3 linker (SEQ ID NO: 4537)), VL of first binding domain, second peptide linker (e.g., a (G4S) linker (SEQ ID NO: 4535)), VL of the second binding domain and CL; and
(ii) a second polypeptide comprising, from N-terminal to C-terminal: VH of the second binding domain, CH1, CH2, and CH3.
   541. The AAV particle of any one of embodiments 515-539, wherein the encoded bispecific antibody molecule comprises:
(i) a first polypeptide comprising, from N-terminal to C-terminal: a DARPIN, a peptide linker (e.g., a (G4S)3 linker (SEQ ID NO: 4537)), VL of the second binding domain and CL; and
(ii) a second polypeptide comprising from N-terminal to C-terminal: VH of the second binding domain, CH1, CH2, and CH3.
   542. The AAV particle of any one of embodiments 515-539, wherein the encoded bispecific antibody molecule comprises:
(i) a first polypeptide comprising, from N-terminal to C-terminal: a Fyn SH3-derived binding polypeptide, a peptide linker (e.g., a (G4S)3 linker (SEQ ID NO: 4537)), VL of the second binding domain and CL; and
(ii) a second polypeptide comprising from N-terminal to C-terminal: VH of the second binding domain, CH1, CH2, and CH3.
   543. The AAV particle of any one of embodiments 515-539, the encoded bispecific antibody molecule comprises:
(i) a first chain, which comprises, from the N-terminus to the C-terminus, a first anti-HER2 VH comprising the amino acid sequence of SEQ ID NO: 5262, and a heavy chain constant region comprising the amino acid sequence of SEQ ID NO: 5216; and a second chain, which comprises from the N-terminus to the C-terminus, a second anti-HER2 VH comprising the amino sequence of 5290, a (G4S)3 linker (SEQ ID NO: 4537), a first anti-HER2 VL comprising the amino acid sequence of SEQ ID NO: 5266, a (GS) linker, a second anti-HER2 VL comprising the amino acid sequence of SEQ ID NO: 5354, and a light chain constant region (CL) comprising the amino acid sequence of SEQ ID NO: 5218; or a sequence substantially identical (e.g., having at least 70%, 75%, 80%, 85%, 90%, 95%, or 99% sequence identity) to the any of the aforesaid sequences;
(ii) a first chain comprising a heavy chain comprising the amino acid sequence of SEQ ID NO: 5264; and a second chain comprising an scFv comprising the amino acid sequence of SEQ ID NO: 5351, fused to a light chain comprising the amino acid sequence of SEQ ID NO: 5268; or a sequence substantially identical (e.g., having at least 70%, 75%, 80%, 85%, 90%, 95%, or 99% sequence identity) to the any of the aforesaid sequences; and/or
(iii) a first chain comprising the amino acid sequence of SEQ ID NO: 5264, or an amino acid sequence substantially identical (e.g., having at least 70%, 75%, 80%, 85%, 90%, 95%, or 99% sequence identity) thereto; and/or a second chain comprising the amino acid sequence of SEQ ID NO: 5365, or an amino acid sequence substantially identical (e.g., having at least 70%, 75%, 80%, 85%, 90%, 95%, or 99% sequence identity) thereto.
   544. The AAV particle of any one of embodiments 515-539 the encoded bispecific antibody molecule comprises:
(i) a first chain, which comprises, from the N-terminus to the C-terminus, a first anti-HER2 VH comprising the amino acid sequence of SEQ ID NO: 5262, and a heavy chain constant region comprising the amino acid sequence of SEQ ID NO: 5220; and a second chain, which comprises from the N-terminus to the C-terminus, a second anti-HER2 VH comprising the amino sequence of 5290, a (G4S)3 linker (SEQ ID NO: 4537), a first anti-HER2 VL comprising the amino acid sequence of SEQ ID NO: 5266, a (GS) linker, a second anti-HER2 VL comprising the amino acid sequence of SEQ ID NO: 5354, and a light chain constant region (CL) comprising the amino acid sequence of SEQ ID NO: 5218; or a sequence substantially identical (e.g., having at least 70%, 75%, 80%, 85%, 90%, 95%, or 99% sequence identity) to the any of the aforesaid sequences;
(ii) a first chain comprising a heavy chain comprising the amino acid sequence of SEQ ID NO: 5376; and a second chain comprising an scFv comprising the amino acid sequence of SEQ ID NO: 5351, fused to a light chain comprising the amino acid sequence of SEQ ID NO: 5268; or a sequence substantially identical (e.g., having at least 70%, 75%, 80%, 85%, 90%, 95%, or 99% sequence identity) to the any of the aforesaid sequences; and/or
(iii) a first chain comprising the amino acid sequence of SEQ ID NO: 5376, or an amino acid sequence substantially identical (e.g., having at least 70%, 75%, 80%, 85%, 90%, 95%, or 99% sequence identity) thereto; and/or a second chain comprising the amino acid sequence of SEQ ID NO: 5365, or an amino acid sequence substantially identical (e.g., having at least 70%, 75%, 80%, 85%, 90%, 95%, or 99% sequence identity) thereto.
   545. The AAV particle of any one of embodiments 515-539, the encoded bispecific antibody molecule comprises:
(i) a first chain, comprising from the N-terminus to the C-terminus, an anti-HER2 VH comprising the amino sequence comprising the amino acid sequence of SEQ ID NO: 5001, and a heavy chain constant region comprising the amino acid sequence of SEQ ID NO: 5018; and a second chain, comprising from the N-terminus to the C-terminus, a DARPIN molecule comprising the amino acid sequence of SEQ ID NO: 5370, a (G4S)3 linker (SEQ ID NO: 4537), an anti-HER2 VL comprising the amino acid sequence of SEQ ID NO: 5006, and a light chain constant region (CL) comprising the amino acid sequence of SEQ ID NO: 5008; or a sequence substantially identical (e.g., having at least 70%, 75%, 80%, 85%, 90%, 95%, or 99% sequence identity) to the any of the aforesaid sequences;
(ii) a first chain comprising the amino acid sequence of SEQ ID NO: 5368; and a second chain comprising the amino acid sequence of SEQ ID NO: 5370, a (G4S)3 linker (SEQ ID NO: 4537), and the amino acid sequence comprising of SEQ ID NO: 5063; or a sequence substantially identical (e.g., having at least 70%, 75%, 80%, 85%, 90%, 95%, or 99% sequence identity) to the any of the aforesaid sequences; and/or
(iii) a first chain comprising the amino acid sequence of SEQ ID NO: 5368, and a second chain comprising the amino acid sequence of SEQ ID NO: 5372; or a sequence substantially identical (e.g., having at least 70%, 75%, 80%, 85%, 90%, 95%, or 99% sequence identity) to the any of the aforesaid sequences.
   546. The AAV particle of any one of embodiments 515-539, the encoded bispecific antibody molecule comprises:
(i) a first chain, comprising from the N-terminus to the C-terminus, an anti-HER2 VH comprising the amino sequence comprising the amino acid sequence of SEQ ID NO: 5010, and a heavy chain constant region comprising the amino acid sequence of SEQ ID NO: 5012; and a second chain, comprising from the N-terminus to the C-terminus, a C12 fynomer comprising the amino acid sequence of SEQ ID NO: 5156, a (G4S)3 linker (SEQ ID NO: 4537), an anti-HER2 VL comprising the amino acid sequence of SEQ ID NO: 5014, and a light chain constant region (CL) comprising the amino acid sequence of SEQ ID NO: 5008; or a sequence substantially identical (e.g., having at least 70%, 75%, 80%, 85%, 90%, 95%, or 99% sequence identity) to the any of the aforesaid sequences; and/or
(ii) a first chain comprising the amino acid sequence of SEQ ID NO: 5066; and a second chain comprising the amino acid sequence of SEQ ID NO: 5156, a (G4S)3 linker (SEQ ID NO: 4537), and the amino acid sequence comprising of SEQ ID NO: 5068; or a sequence substantially identical (e.g., having at least 70%, 75%, 80%, 85%, 90%, 95%, or 99% sequence identity) to the any of the aforesaid sequences.
   547. The AAV particle of any one of embodiments 422-546, further comprising a genetic element comprising a promoter operably linked to the transgene.
   548. The AAV particle of embodiment 547, wherein:
(i) the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), or a fragment, e.g., a truncation, or a functional variant thereof; and/or
(ii) the promoter comprises a nucleotide sequence chosen from any one of SEQ ID NOs: 6563-6572, 6593-6594, or 4599, or a nucleotide sequence with at least 95% sequence identity thereto.
   549. The AAV particle of embodiment 548, wherein:
(i) the promoter is selected from a CAG promoter, a CBA promoter (e.g., a minimal CBA promoter), a CB promoter, a CMV(IE) promoter and/or enhancer, a GFAP promoter, a synapsin promoter, an ICAM2 promoter, or a functional variant thereof; and/or
(ii) the promoter comprises a nucleotide sequence selected from any one of SEQ ID NOs: 6565, 6566, 6568, 6569, 4599, 6594, or a nucleotide sequence with at least 95% sequence identity thereto.
   550. The AAV particle of embodiment 547, wherein the promoter is a ubiquitous promoter.
   551. The AAV particle of embodiment 550, wherein the ubiquitous promoter is selected from CMV, CBA (including derivatives CAG, CB6, CBh, etc.), EF-1α, PGK, UBC, GUSB (hGBp), or UCOE (promoter of HNRPA2B1-CBX3).
   552. The AAV particle of embodiment 547, wherein the promoter is a tissue specific promoter, e.g., a GFAP promoter or a synapsin promoter.
   553. The AAV particle of any one of embodiments 547-550, wherein the promoter:
(i) is a CB promoter; and/or
(ii) comprises the nucleotide sequence of SEQ ID NO: 6566 or a nucleotide sequence with at least 95% sequence identity thereto.
   554. The AAV particle of any one of embodiments 547-550, wherein the promoter:
(i) is a CMV(IE) promoter; and/or
(ii) comprises the nucleotide sequence of SEQ ID NO: 6594, or a nucleotide sequence with at least 95% sequence identity thereto.
   555. The AAV particle of any one of embodiments 547, 548, or 552, wherein the promoter:
(i) is a GFAP promoter; and/or
(ii) comprises the nucleotide sequence of SEQ ID NO: 6568, or a nucleotide sequence with at least 95% sequence identity thereto.
   556. The AAV particle of any one of embodiments 547-555, wherein the genetic element further comprises a CMV immediate-early (CMVie) enhancer, optionally wherein the CMVie enhancer comprises the nucleotide sequence of SEQ ID NO: 6564, or a nucleotide sequence with at least 95% sequence identity to SEQ ID NO: 6564.
   557. The AAV particle of any one of embodiments 547-556, wherein the genetic element further comprises a polyadenylation (polyA) signal region, optionally wherein the polyA signal region comprises a rabbit globin polyA signal region.
   558. The AAV particle of embodiment 557, wherein the polyA signal region comprises:
(i) the nucleotide sequence of any of SEQ ID NOs: 6590-6592, or a nucleotide sequence with at least 95% sequence identity to any of SEQ ID NOs: 6590-6592; or
(ii) the nucleotide sequence of SEQ ID NO: 6590, or a nucleotide sequence with at least 95% sequence identity thereto.
   559. The AAV particle of any one of embodiments 547-558, wherein the genetic element further comprises an inverted terminal repeat (ITR) sequence, optionally wherein the ITR sequence is positioned 5′ relative to the encoded transgene and/or ITR sequence is positioned 3′ relative to the encoded transgene.
   560. The AAV particle of any one of embodiments 559, wherein the ITR sequence comprises a nucleotide sequence of any one of SEQ ID NOs: 6559-6562, or a nucleotide sequence with at least 95% sequence identity thereto.
   561. The AAV particle of embodiment 559 or 560, wherein:
(i) the ITR sequence positioned 5′ relative to the encoded transgene comprises the nucleotide sequence of SEQ ID NO: 6559, or a nucleotide sequence with at least 95% sequence identity thereto; and/or
(ii) the ITR sequence positioned 3′ relative to the encoded transgene comprises the nucleotide sequence of SEQ ID NO: 6561, or a nucleotide sequence with at least 95% sequence identity thereto.
   562. The AAV particle of any one of embodiments 547-561, wherein the genetic element further comprises an intron region, optionally wherein the intron region comprises a nucleotide sequence of any one of SEQ ID NOs: 6578-6588, 6595, 6607, 6608, or 6609, or a nucleotide sequence with at least 95% identity thereto.
   563. The AAV particle of embodiment 562, wherein the intron region comprises a human beta-globin intron region, optionally wherein the human beta-globin intron region comprises the nucleotide sequence of SEQ ID NO: 6580 or 6595, or a nucleotide sequence with at least 95% sequence identity thereto.
   564. The AAV particle of embodiment 562, wherein the intron region comprises an ie intron 1 region, optionally wherein the ie intron 1 region comprises the nucleotide sequence of SEQ ID NO: 6578, or a nucleotide sequence with at least 95% sequence identity thereto.
   565. The AAV particle of any one of embodiments 547-564, wherein the genetic element comprises at least 2 intron regions.
   566. The AAV particle of any one of embodiments 547-565, wherein the genetic element comprises:
(i) an intron region comprising the nucleotide sequence of SEQ ID NO: 6578 and SEQ ID NO: 6580;
(ii) an intron region comprising the nucleotide sequence of SEQ ID NO: 6578 and 6595;
(iii) an ie intron 1 region and a human beta-globin intron region;
(iv) an intron region comprising the nucleotide sequence of SEQ ID NO: 6580 and 6595; or
(v) an intron region comprising the nucleotide sequence of 6595.
   567. The AAV particle of any one of embodiments 547-566, wherein the genetic element further comprises an exon region, optionally wherein the exon region comprises a nucleotide sequence of any of SEQ ID NOs: 6573-6577, or a sequence with at least 95% sequence identity thereto.
   568. The AAV particle of embodiment 567, wherein the exon region comprises an ie exon 1 region, optionally wherein the ie exon 1 region comprises the nucleotide sequence of SEQ ID NO: 6573, or a nucleotide sequence with at least 95% sequence identity thereto.
   569. The AAV particle of any one of embodiments 547-568, wherein the genetic element further comprises a Kozak sequence, optionally wherein the Kozak sequence comprises:
(i) the nucleotide sequence of GCCGCCACCATG (SEQ ID NO: 6589) or GAGGAGCCACC (SEQ ID NO: 4543); or
(ii) the nucleotide sequence of GCCGCCACCATG (SEQ ID NO: 6589).
   570. The AAV particle of any one of embodiments 547-569, wherein the genetic element further comprises a nucleotide sequence encoding a miR binding site, e.g., a miR binding site that modulates, e.g., reduces, expression of the antibody molecule encoded by the genetic element in a cell or tissue where the corresponding miRNA is expressed.
   571. The AAV particle of embodiment 570, wherein the encoded miRNA binding site is complementary, e.g., fully complementary or partially complementary, to a miRNA expressed in a cell or tissue of the DRG, liver, heart, hematopoietic, or a combination thereof.
   572. The AAV particle of embodiment 570 or 571, wherein the encoded miR binding site modulates, e.g., reduces, expression of the encoded antibody molecule in a cell or tissue of the DRG, liver, heart, hematopoietic lineage, or a combination thereof.
   573. The AAV particle of any one of embodiments 547-572, wherein the genetic element comprises at least 1-5 copies of the encoded miR binding site, e.g., at least 1, 2, 3, 4, or 5 copies.
   574. The AAV particle of any one of embodiments 547-573, wherein the genetic element comprises at least 3 copies of an encoded miR binding sites, optionally wherein all three copies comprise the same miR binding site, or at least one, two, three, or all of the copies comprise a different miR binding site.
   575. The AAV particle of embodiment 574, wherein the 3 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, but no more than four modifications of GATAGTTA.
   576. The AAV particle of any one of embodiments 547-573, wherein the genetic element comprises at least 4 copies of an encoded miR binding site, optionally wherein all four copies comprise the same miR binding site, or at least one, two, three, or all of the copies comprise a different miR binding site, optionally wherein the 4 copies of the encoded miR binding sites are continuous (e.g., not separated by a spacer), or are separated by a spacer, and further optionally wherein the spacer comprises the nucleotide sequence of GATAGTTA, or a nucleotide sequence having at least one, two, or three modifications, but no more than four modifications of GATAGTTA.
   577. The AAV particle of any one of embodiments 570-576, wherein the encoded miR binding site comprises a miR122 binding site, a miR183 binding site, a miR-142-3p binding site, a mir-1 binding site, or a combination thereof, optionally wherein:
(i) the encoded miR122 binding site comprises the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673;
(ii) the encoded miR183 binding site comprises the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4676; (iii) the encoded miR-142-3p binding site comprises the nucleotide sequence of SEQ ID NO: 4675, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4675; and/or
(iv) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, e.g., substitutions (e.g., conservative substitutions), but no more than ten modifications, e.g., substitutions (e.g., conservative substitutions), relative to SEQ ID NO: 4679.
   578. The AAV particle of any one of embodiments 547-577, wherein the genetic element comprises:
(i) an encoded miR122 binding site;
(ii) an encoded mir122 binding site, an encoded mir-1 binding site, and/or an encoded mir-183 binding site.
   579. The AAV particle of any one of embodiments 547-578, wherein the genetic element comprises at least 1-5 copies, e.g., 1, 2, or 3 copies of a miR122 binding site, a mir-1 binding site, or a combination thereof, optionally wherein each copy is continuous (e.g., not separated by a spacer), or each copy is separated by a spacer, optionally wherein the spacer comprises the nucleotide sequence of GATAGTTA, or a nucleotide sequence having at least one, two, or three modifications, but no more than four modifications of GATAGTTA.
   580. The AAV particle of embodiment 578 or 579, wherein:
(i) the encoded miR122 binding site comprises the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673; and/or
(ii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4679.
   581. The AAV particle of any one of embodiments 547-580, wherein the genetic element comprises:
(A) (i) a first encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673;
   (ii) a first spacer comprising the nucleotide sequence of GATAGTTA, or a nucleotide sequence having at least one, two, or three modifications, but no more than four modifications of GATAGTTA; and
   (iii) a second encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673; or
(B) (i) a first encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673;
   (ii) a first spacer comprising the nucleotide sequence of GATAGTTA, or a nucleotide sequence having at least one, two, or three modifications, but no more than four modifications of GATAGTTA;
   (iii) a second encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673;
   (iv) a second spacer comprising the nucleotide sequence of GATAGTTA, or a nucleotide sequence having at least one, two, or three modifications, but no more than four modifications of GATAGTTA; and
   (v) a third encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 4673.
      582. The AAV particle of any of one of embodiments 547-581, wherein the genetic element comprises:
(i) the nucleotide sequence of any of SEQ ID NOs: 5163-5179 5185-5190, 5343, 5374, 5375, 6500, 6501, 6502, 6503, 6504, 6505, 6506, 6507, 6508, or 6509 or a sequence with at least 95% sequence identity thereto; and/or
(ii) is single stranded.
   583. The AAV particle of any one of embodiments 547-582, wherein the genetic element is self-complementary.
   584. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5002;
(viii) an encoded heavy chain constant region, optionally wherein the nucleotide sequence encoding the heavy chain constant region comprises the nucleotide sequence of SEQ ID NO: 5003;
(ix) an encoded furin cleavage site, optionally wherein the nucleotide sequence encoding the furin cleavage site comprises the nucleotide sequence of SEQ ID NO: 1724;
(x) an encoded T2A linker, optionally wherein the nucleotide sequence encoding the T2A linker comprises the nucleotide sequence of SEQ ID NO: 1726;
(xi) a signal sequence, optionally wherein the signal sequence comprises the nucleotide sequence of SEQ ID NO: 5159;
(xii) a transgene encoding a light chain variable region (VL) comprising a nucleotide sequence with at least 90% (e.g., at least about 95, 96, 97, 98, or 99%) sequence identity to the nucleotide sequence of SEQ ID NO: 5005;
(xiii) an encoded light chain constant region, optionally wherein the nucleotide sequence encoding the light chain constant region comprises the nucleotide sequence of SEQ ID NO: 5007;
(xiv) a rabbit globin polyA signal region, optionally wherein the rabbit globin polyA signal region comprises the nucleotide sequence of SEQ ID NO: 6590; and
(xv) a 3′ AAV ITR, optionally wherein the 3′ AAV ITR comprises the nucleotide sequence of SEQ ID NO: 6561.
   585. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5171;
(viii) an encoded heavy chain constant region, optionally wherein the nucleotide sequence encoding the heavy chain constant region comprises the nucleotide sequence of SEQ ID NO: 5011;
(ix) an encoded furin cleavage site, optionally wherein the nucleotide sequence encoding the furin cleavage site comprises the nucleotide sequence of SEQ ID NO: 1724;
(x) an encoded T2A linker, optionally wherein the nucleotide sequence encoding the T2A linker comprises the nucleotide sequence of SEQ ID NO: 1726;
(xi) a signal sequence, optionally wherein the signal sequence comprises the nucleotide sequence of SEQ ID NO: 5159;
(xii) a transgene encoding a light chain variable region (VL) comprising a nucleotide sequence with at least 90% (e.g., at least about 95, 96, 97, 98, or 99%) sequence identity to the nucleotide sequence of SEQ ID NO: 5175;
(xiii) an encoded light chain constant region, optionally wherein the nucleotide sequence encoding the light chain constant region comprises the nucleotide sequence of SEQ ID NO: 5007;
(xiv) a rabbit globin polyA signal region, optionally wherein the rabbit globin polyA signal region comprises the nucleotide sequence of SEQ ID NO: 6590; and
(xv) a 3′ AAV ITR, optionally wherein the 3′ AAV ITR comprises the nucleotide sequence of SEQ ID NO: 6561.
   586. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CMV(IE) promoter and/or enhancer, optionally wherein the CMV(IE) promoter and/or enhancer comprises the nucleotide sequence of SEQ ID NO: 6594;
(iii) a CB promoter, optionally wherein the CB promoter comprises the nucleotide sequence of SEQ ID NO: 6566;
(iv) a human beta-globin intron region, optionally wherein the human beta-globin intron region comprises the nucleotide sequence of SEQ ID NO: 6595;
(v) a signal sequence, optionally wherein the signal sequence comprises the nucleotide sequence of SEQ ID NO: 5157;
(vi) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5009;
(vii) an encoded heavy chain constant region, optionally wherein the nucleotide sequence encoding the heavy chain constant region comprises the nucleotide sequence of SEQ ID NO: 5011;
(viii) an encoded furin cleavage site, optionally wherein the nucleotide sequence encoding the furin cleavage site comprises the nucleotide sequence of SEQ ID NO: 1724;
(ix) an encoded T2A linker, optionally wherein the nucleotide sequence encoding the T2A linker comprises the nucleotide sequence of SEQ ID NO: 1726;
(x) a signal sequence, optionally wherein the signal sequence comprises the nucleotide sequence of SEQ ID NO: 5159;
(xi) a C12 Fynomer sequence, optionally wherein the C12 Fynomer sequence comprises the nucleotide sequence of SEQ ID NO: 5155;
(xii) an encoded glycine-serine linker, optionally wherein the nucleotide sequence encoding the glycine-serine linker comprises the nucleotide sequence of SEQ ID NO: 5347;
(xiii) a transgene encoding a light chain variable region (VL) comprising a nucleotide sequence with at least 90% (e.g., at least about 95, 96, 97, 98, or 99%) sequence identity to the nucleotide sequence of SEQ ID NO: 5013;
(xiv) an encoded light chain constant region, optionally wherein the nucleotide sequence encoding the light chain constant region comprises the nucleotide sequence of SEQ ID NO: 5007;
(xv) a rabbit globin polyA signal region, optionally wherein the rabbit globin polyA signal region comprises the nucleotide sequence of SEQ ID NO: 6590; and
(xvi) a 3′ AAV ITR, optionally wherein the 3′ AAV ITR comprises the nucleotide sequence of SEQ ID NO: 6561.
   587. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CMV(IE) promoter and/or enhancer, optionally wherein the CMV(IE) promoter and/or enhancer comprises the nucleotide sequence of SEQ ID NO: 6594;
(iii) a CB promoter, optionally wherein the CB promoter comprises the nucleotide sequence of SEQ ID NO: 6566;
(iv) an intron region, optionally wherein the intron region comprises the nucleotide sequence of SEQ ID NO: 6595;
(v) a signal sequence, optionally wherein the signal sequence comprises the nucleotide sequence of SEQ ID NO: 5157;
(vi) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5015;
(vii) an encoded heavy chain constant region, optionally wherein the nucleotide sequence encoding the heavy chain constant region comprises the nucleotide sequence of SEQ ID NO: 5017;
(viii) an encoded furin cleavage site, optionally wherein the nucleotide sequence encoding the furin cleavage site comprises the nucleotide sequence of SEQ ID NO: 1724;
(ix) an encoded T2A linker, optionally wherein the nucleotide sequence encoding the T2A linker comprises the nucleotide sequence of SEQ ID NO: 1726;
(x) a signal sequence, optionally wherein the signal sequence comprises the nucleotide sequence of SEQ ID NO: 5159;
(xi) a transgene encoding a light chain variable region (VL) comprising a nucleotide sequence with at least 90% (e.g., at least about 95, 96, 97, 98, or 99%) sequence identity to the nucleotide sequence of SEQ ID NO: 5019;
(xii) an encoded light chain constant region, optionally wherein the nucleotide sequence encoding the light chain constant region comprises the nucleotide sequence of SEQ ID NO: 5021;
(xiii) a rabbit globin polyA signal region, optionally wherein the rabbit globin polyA signal region comprises the nucleotide sequence of SEQ ID NO: 6590; and
(xiv) a 3′ AAV ITR, optionally wherein the 3′ AAV ITR comprises the nucleotide sequence of SEQ ID NO: 6561.
   588. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CMV(IE) promoter and/or enhancer, optionally wherein the CMV(IE) promoter and/or enhancer comprises the nucleotide sequence of SEQ ID NO: 6594;
(iii) a CB promoter, optionally wherein the CB promoter comprises the nucleotide sequence of SEQ ID NO: 6566;
(iv) an intron region, optionally wherein the intron region comprises the nucleotide sequence of SEQ ID NO: 6595;
(v) a signal sequence, optionally wherein the signal sequence comprises the nucleotide sequence of SEQ ID NO: 5157;
(vi) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5070;
(vii) an encoded heavy chain constant region, optionally wherein the nucleotide sequence encoding the heavy chain constant region comprises the nucleotide sequence of SEQ ID NO: 5025;
(viii) an encoded furin cleavage site, optionally wherein the nucleotide sequence encoding the furin cleavage site comprises the nucleotide sequence of SEQ ID NO: 1724;
(ix) an encoded T2A linker, optionally wherein the nucleotide sequence encoding the T2A linker comprises the nucleotide sequence of SEQ ID NO: 1726;
(x) a signal sequence, optionally wherein the signal sequence comprises the nucleotide sequence of SEQ ID NO: 5159;
(xi) a transgene encoding a light chain variable region (VL) comprising a nucleotide sequence with at least 90% (e.g., at least about 95, 96, 97, 98, or 99%) sequence identity to the nucleotide sequence of SEQ ID NO: 5085;
(xii) an encoded light chain constant region, optionally wherein the nucleotide sequence encoding the light chain constant region comprises the nucleotide sequence of SEQ ID NO: 5007;
(xiii) a rabbit globin polyA signal region, optionally wherein the rabbit globin polyA signal region comprises the nucleotide sequence of SEQ ID NO: 6590; and
(xiv) a 3′ AAV ITR, optionally wherein the 3′ AAV ITR comprises the nucleotide sequence of SEQ ID NO: 6561.
   589. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CMV(IE) promoter and/or enhancer, optionally wherein the CMV(IE) promoter and/or enhancer comprises the nucleotide sequence of SEQ ID NO: 6594;
(iii) a CB promoter, optionally wherein the CB promoter comprises the nucleotide sequence of SEQ ID NO: 6566;
(iv) an intron region, optionally wherein the intron region comprises the nucleotide sequence of SEQ ID NO: 6595;
(v) a signal sequence, optionally wherein the signal sequence comprises the nucleotide sequence of SEQ ID NO: 5157;
(vi) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5089;
(vii) an encoded heavy chain constant region, optionally wherein the nucleotide sequence encoding the heavy chain constant region comprises the nucleotide sequence of SEQ ID NO: 5029;
(viii) an encoded furin cleavage site, optionally wherein the nucleotide sequence encoding the furin cleavage site comprises the nucleotide sequence of SEQ ID NO: 1724;
(ix) an encoded T2A linker, optionally wherein the nucleotide sequence encoding the T2A linker comprises the nucleotide sequence of SEQ ID NO: 1726;
(x) a signal sequence, optionally wherein the signal sequence comprises the nucleotide sequence of SEQ ID NO: 5159;
(xi) a transgene encoding a light chain variable region (VL) comprising a nucleotide sequence with at least 90% (e.g., at least about 95, 96, 97, 98, or 99%) sequence identity to the nucleotide sequence of SEQ ID NO: 5093;
(xii) an encoded light chain constant region, optionally wherein the nucleotide sequence encoding the light chain constant region comprises the nucleotide sequence of SEQ ID NO: 5007;
(xiii) a rabbit globin polyA signal region, optionally wherein the rabbit globin polyA signal region comprises the nucleotide sequence of SEQ ID NO: 6590; and
(xiv) a 3′ AAV ITR, optionally wherein the 3′ AAV ITR comprises the nucleotide sequence of SEQ ID NO: 6561.
   590. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CMV(IE) promoter and/or enhancer, optionally wherein the CMV(IE) promoter and/or enhancer comprises the nucleotide sequence of SEQ ID NO: 6594;
(iii) a CB promoter, optionally wherein the CB promoter comprises the nucleotide sequence of SEQ ID NO: 6566;
(iv) an intron region, optionally wherein the intron region comprises the nucleotide sequence of SEQ ID NO: 6595;
(v) a signal sequence, optionally wherein the signal sequence comprises the nucleotide sequence of SEQ ID NO: 5157;
(vi) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5109;
(vii) an encoded heavy chain constant region, optionally wherein the nucleotide sequence encoding the heavy chain constant region comprises the nucleotide sequence of SEQ ID NO: 5017;
(viii) an encoded furin cleavage site, optionally wherein the nucleotide sequence encoding the furin cleavage site comprises the nucleotide sequence of SEQ ID NO: 1724;
(ix) an encoded T2A linker, optionally wherein the nucleotide sequence encoding the T2A linker comprises the nucleotide sequence of SEQ ID NO: 1726;
(x) a signal sequence, optionally wherein the signal sequence comprises the nucleotide sequence of SEQ ID NO: 5159;
(xi) a transgene encoding a light chain variable region (VL) comprising a nucleotide sequence with at least 90% (e.g., at least about 95, 96, 97, 98, or 99%) sequence identity to the nucleotide sequence of SEQ ID NO: 5113;
(xii) an encoded light chain constant region, optionally wherein the nucleotide sequence encoding the light chain constant region comprises the nucleotide sequence of SEQ ID NO: 5007;
(xiii) a rabbit globin polyA signal region, optionally wherein the rabbit globin polyA signal region comprises the nucleotide sequence of SEQ ID NO: 6590; and
(xiv) a 3′ AAV ITR, optionally wherein the 3′ AAV ITR comprises the nucleotide sequence of SEQ ID NO: 6561.
   591. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5191;
(viii) an encoded heavy chain constant region, optionally wherein the nucleotide sequence encoding the heavy chain constant region comprises the nucleotide sequence of SEQ ID NO: 5211;
(ix) an encoded furin cleavage site, optionally wherein the nucleotide sequence encoding the furin cleavage site comprises the nucleotide sequence of SEQ ID NO: 1724;
(x) an encoded T2A linker, optionally wherein the nucleotide sequence encoding the T2A linker comprises the nucleotide sequence of SEQ ID NO: 1726;
(xi) a signal sequence, optionally wherein the signal sequence comprises the nucleotide sequence of SEQ ID NO: 5159;
(xii) a transgene encoding a light chain variable region (VL) comprising a nucleotide sequence with at least 90% (e.g., at least about 95, 96, 97, 98, or 99%) sequence identity to the nucleotide sequence of SEQ ID NO: 5195;
(xiii) an encoded light chain constant region, optionally wherein the nucleotide sequence encoding the light chain constant region comprises the nucleotide sequence of SEQ ID NO: 5007;
(xiv) a rabbit globin polyA signal region, optionally wherein the rabbit globin polyA signal region comprises the nucleotide sequence of SEQ ID NO: 6590; and
(xv) a 3′ AAV ITR, optionally wherein the 3′ AAV ITR comprises the nucleotide sequence of SEQ ID NO: 6561.
   592. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5223;
(viii) an encoded heavy chain constant region, optionally wherein the nucleotide sequence encoding the heavy chain constant region comprises the nucleotide sequence of SEQ ID NO: 5213;
(ix) an encoded furin cleavage site, optionally wherein the nucleotide sequence encoding the furin cleavage site comprises the nucleotide sequence of SEQ ID NO: 1724;
(x) an encoded T2A linker, optionally wherein the nucleotide sequence encoding the T2A linker comprises the nucleotide sequence of SEQ ID NO: 1726;
(xi) a signal sequence, optionally wherein the signal sequence comprises the nucleotide sequence of SEQ ID NO: 5159;
(xii) a transgene encoding a light chain variable region (VL) encoded by a nucleotide sequence comprising a nucleotide sequence with at least 90% (e.g., at least about 95, 96, 97, 98, or 99%) sequence identity to the nucleotide sequence of SEQ ID NO: 5227;
(xiii) an encoded light chain constant region, optionally wherein the nucleotide sequence encoding the light chain constant region comprises the nucleotide sequence of SEQ ID NO: 5007;
(xiv) a rabbit globin polyA signal region, optionally wherein the rabbit globin polyA signal region comprises the nucleotide sequence of SEQ ID NO: 6590; and
(xv) a 3′ AAV ITR, optionally wherein the 3′ AAV ITR comprises the nucleotide sequence of SEQ ID NO: 6561.
   593. The AAV particle of any one of embodiments 547-583, wherein the genetic element comprises in 5′ to 3′ order:
(i) (i) a 5′ adeno-associated (AAV) ITR, optionally wherein the 5′ AAV ITR comprises the nucleotide sequence of SEQ ID NO: 6559;
(ii) a CMV(IE) promoter and/or enhancer, optionally wherein the CMV(IE) promoter and/or enhancer comprises the nucleotide sequence of SEQ ID NO: 6594;
(iii) a CB promoter, optionally wherein the CB promoter comprises the nucleotide sequence of SEQ ID NO: 6566;
(iv) ie exon 1 region, optionally wherein the ie exon 1 region comprises the nucleotide sequence of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5253;
(viii) an encoded glycine-serine linker, optionally wherein the glycine-serine linker is encoded by the nucleotide sequence of SEQ ID NO: 5347;
(ix) a transgene encoding a light chain variable region (VL) comprising a nucleotide sequence with at least 90% (e.g., at least about 95, 96, 97, 98, or 99%) sequence identity to the nucleotide sequence of SEQ ID NO: 5255;
(x) an encoded light chain constant region, optionally wherein the nucleotide sequence encoding the light chain constant region comprises the nucleotide sequence of SEQ ID NO: 5277;
(xi) a rabbit globin polyA signal region, optionally wherein the rabbit globin polyA signal region comprises the nucleotide sequence of SEQ ID NO: 6590; and
(xii) a 3′ AAV ITR, optionally wherein the 3′ AAV ITR comprises the nucleotide sequence of SEQ ID NO: 6561.
   594. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a GFAP promoter and/or enhancer, optionally wherein the GFAP comprises the nucleotide sequence of SEQ ID NO: 6568;
(iii) a human beta-globin intron region, optionally wherein the human beta-globin intron region comprises the nucleotide sequence of SEQ ID NO 6595;
(iv) a signal sequence, optionally wherein the signal sequence comprises the nucleotide sequence of SEQ ID NO: 5157;
(v) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5129;
(vi) an encoded heavy chain constant region, optionally wherein the nucleotide sequence encoding the heavy chain constant region comprises the nucleotide sequence of SEQ ID NO: 5017;
(vii) an encoded furin cleavage site, optionally wherein the nucleotide sequence encoding the furin cleavage site comprises the nucleotide sequence of SEQ ID NO: 1724;
(viii) an encoded T2A linker, optionally wherein the nucleotide sequence encoding the T2A linker comprises the nucleotide sequence of SEQ ID NO: 1726;
(ix) a signal sequence, optionally wherein the signal sequence comprises the nucleotide sequence of SEQ ID NO: 5159;
(x) a transgene encoding a light chain variable region (VL) comprising a nucleotide sequence with at least 90% (e.g., at least about 95, 96, 97, 98, or 99%) sequence identity to the nucleotide sequence of SEQ ID NO: 5133;
(xi) an encoded light chain constant region, optionally wherein the nucleotide sequence encoding the light chain constant region comprises the nucleotide sequence of SEQ ID NO: 5021;
(xii) a rabbit globin polyA signal region, optionally wherein the rabbit globin polyA signal region comprises the nucleotide sequence of SEQ ID NO: 6590; and
(xiii) a 3′ AAV ITR, optionally wherein the 3′ AAV ITR comprises the nucleotide sequence of SEQ ID NO: 6561.
   595. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) comprising a nucleotide sequence with at least 90% (e.g., at least about 95, 96, 97, 98, or 99%) sequence identity to the nucleotide sequence of SEQ ID NO: 5257;
(viii) an encoded glycine-serine linker, optionally wherein the glycine-serine linker is encoded by the nucleotide sequence of SEQ ID NO: 5347;
(xii) a transgene encoding a light chain variable region (VL) comprising a nucleotide sequence with at least 90% (e.g., at least about 95, 96, 97, 98, or 99%) sequence identity to the nucleotide sequence of SEQ ID NO: 5259;
(xiii) an encoded light chain constant region, optionally wherein the nucleotide sequence encoding the light chain constant region comprises the nucleotide sequence of SEQ ID NO: 5279;
(ix) a rabbit globin polyA signal region, optionally wherein the rabbit globin polyA signal region comprises the nucleotide sequence of SEQ ID NO: 6590; and
(x) a 3′ AAV ITR, optionally wherein the 3′ AAV ITR comprises the nucleotide sequence of SEQ ID NO: 6561.
   596. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5261;
(viii) an encoded heavy chain constant region, optionally wherein the heavy chain constant region is encoded by the nucleotide sequence of SEQ ID NO: 5215;
(ix) an encoded furin cleavage site, optionally wherein the nucleotide sequence encoding the furin cleavage site comprises the nucleotide sequence of SEQ ID NO: 6557;
(x) an encoded T2A linker, optionally wherein the nucleotide sequence encoding the T2A linker comprises the nucleotide sequence of SEQ ID NO: 6558;
(xi) a signal sequence, optionally wherein the signal sequence comprises the nucleotide sequence of SEQ ID NO: 5159;
(xii) a transgene encoding a heavy chain variable region comprising a nucleotide sequence with at least 90% (e.g., at least about 95, 96, 97, 98, or 99%) sequence identity to the nucleotide sequence of SEQ ID NO: 5289;
(xiii) an encoded glycine-serine linker, optionally wherein the glycine-serine linker is encoded by the nucleotide sequence of SEQ ID NO: 5347;
(xiv) a transgene encoding a light chain variable region (VL) comprising a nucleotide sequence with at least 90% (e.g., at least about 95, 96, 97, 98, or 99%) sequence identity to the nucleotide sequence of SEQ ID NO: 6552;
(xv) an encoded glycine-serine linker, optionally wherein the glycine-serine linker is encoded by the nucleotide sequence of GGCTCT;
(xvi) a transgene encoding a light chain variable region (VL) comprising a nucleotide sequence with at least 90% (e.g., at least about 95, 96, 97, 98, or 99%) sequence identity to the nucleotide sequence of SEQ ID NO: 5353;
(xvii) an encoded light chain constant region, optionally wherein the nucleotide sequence encoding the light chain constant region comprises a nucleotide sequence with at least 90% (e.g., at least about 95, 96, 97, 98, or 99%) sequence identity to the nucleotide sequence of SEQ ID NO: 5217;
(xviii) a rabbit globin polyA signal region, optionally wherein the rabbit globin polyA signal region comprises the nucleotide sequence of SEQ ID NO: 6590; and
(xix) a 3′ AAV ITR, optionally wherein the 3′ AAV ITR comprises the nucleotide sequence of SEQ ID NO: 6561.
   597. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5032;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5269;
(viii) an encoded heavy chain constant region, optionally wherein the heavy chain constant region is encoded by a nucleotide sequence comprising a nucleotide sequence with at least 90% (e.g., at least about 95, 96, 97, 98, or 99%) sequence identity to the nucleotide sequence of SEQ ID NO: 5219;
(ix) an encoded furin cleavage site, optionally wherein the nucleotide sequence encoding the furin cleavage site comprises the nucleotide sequence of SEQ ID NO: 6557;
(x) an encoded T2A linker, optionally wherein the nucleotide sequence encoding the T2A linker comprises the nucleotide sequence of SEQ ID NO: 6558;
(xi) a signal sequence, optionally wherein the signal sequence comprises the nucleotide sequence of SEQ ID NO: 5159;
(xii) a transgene encoding a light chain variable region (VL) encoded by a nucleotide sequence comprising a nucleotide sequence with at least 90% (e.g., at least about 95, 96, 97, 98, or 99%) sequence identity to the nucleotide sequence of SEQ ID NO: 5273;
(xiii) an encoded light chain constant region, optionally wherein the nucleotide sequence encoding the light chain constant region comprises the nucleotide sequence of SEQ ID NO: 5221;
(ix) a rabbit globin polyA signal region, optionally wherein the rabbit globin polyA signal region comprises the nucleotide sequence of SEQ ID NO: 6590; and
(x) a 3′ AAV ITR, optionally wherein the 3′ AAV ITR comprises the nucleotide sequence of SEQ ID NO: 6561.
   598. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a GFAP promoter, optionally wherein the GFAP promoter comprises the nucleotide sequence of SEQ ID NO: 6568;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5032;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5269;
(viii) an encoded heavy chain constant region, optionally wherein the heavy chain constant region is encoded by a nucleotide sequence comprising a nucleotide sequence with at least 90% (e.g., at least about 95, 96, 97, 98, or 99%) sequence identity to the nucleotide sequence of SEQ ID NO: 7590;
(ix) an encoded furin cleavage site, optionally wherein the nucleotide sequence encoding the furin cleavage site comprises the nucleotide sequence of SEQ ID NO: 6557;
(x) an encoded T2A linker, optionally wherein the nucleotide sequence encoding the T2A linker comprises the nucleotide sequence of SEQ ID NO: 6558;
(xi) a signal sequence, optionally wherein the signal sequence comprises the nucleotide sequence of SEQ ID NO: 5159;
(xii) a transgene encoding a light chain variable region (VL) encoded by a nucleotide sequence comprising a nucleotide sequence with at least 90% (e.g., at least about 95, 96, 97, 98, or 99%) sequence identity to the nucleotide sequence of SEQ ID NO: 5273;
(xiii) an encoded light chain constant region, optionally wherein the nucleotide sequence encoding the light chain constant region comprises the nucleotide sequence of SEQ ID NO: 5221;
(ix) a rabbit globin polyA signal region, optionally wherein the rabbit globin polyA signal region comprises the nucleotide sequence of SEQ ID NO: 6590; and
(x) a 3′ AAV ITR, optionally wherein the 3′ AAV ITR comprises the nucleotide sequence of SEQ ID NO: 6561.
   599. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a GFAP promoter, optionally wherein the GFAP promoter comprises the nucleotide sequence of SEQ ID NO: 6568;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5032;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5269;
(viii) an encoded heavy chain constant region, optionally wherein the heavy chain constant region is encoded by a nucleotide sequence comprising a nucleotide sequence with at least 90% (e.g., at least about 95, 96, 97, 98, or 99%) sequence identity to the nucleotide sequence of SEQ ID NO: 5219;
(ix) an encoded furin cleavage site, optionally wherein the nucleotide sequence encoding the furin cleavage site comprises the nucleotide sequence of SEQ ID NO: 6557;
(x) an encoded T2A linker, optionally wherein the nucleotide sequence encoding the T2A linker comprises the nucleotide sequence of SEQ ID NO: 6558;
(xi) a signal sequence, optionally wherein the signal sequence comprises the nucleotide sequence of SEQ ID NO: 5159;
(xii) a transgene encoding a light chain variable region (VL) encoded by a nucleotide sequence comprising a nucleotide sequence with at least 90% (e.g., at least about 95, 96, 97, 98, or 99%) sequence identity to the nucleotide sequence of SEQ ID NO: 5273;
(xiii) an encoded light chain constant region, optionally wherein the nucleotide sequence encoding the light chain constant region comprises the nucleotide sequence of SEQ ID NO: 5221;
(ix) a rabbit globin polyA signal region, optionally wherein the rabbit globin polyA signal region comprises the nucleotide sequence of SEQ ID NO: 6590; and
(x) a 3′ AAV ITR, optionally wherein the 3′ AAV ITR comprises the nucleotide sequence of SEQ ID NO: 6561.
   600. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5032;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 6512, 6517, 6522, 6527, or 6532;
(viii) an encoded heavy chain constant region, optionally wherein the heavy chain constant region is encoded by a nucleotide sequence comprising a nucleotide sequence with at least 90% (e.g., at least about 95, 96, 97, 98, or 99%) sequence identity to the nucleotide sequence of SEQ ID NO: 5219;
(ix) an encoded furin cleavage site, optionally wherein the nucleotide sequence encoding the furin cleavage site comprises the nucleotide sequence of SEQ ID NO: 1724;
(x) an encoded T2A linker, optionally wherein the nucleotide sequence encoding the T2A linker comprises the nucleotide sequence of SEQ ID NO: 1726;
(xi) a signal sequence, optionally wherein the signal sequence comprises the nucleotide sequence of SEQ ID NO: 5159;
(xii) a transgene encoding a light chain variable region (VL) encoded by a nucleotide sequence comprising a nucleotide sequence with at least 90% (e.g., at least about 95, 96, 97, 98, or 99%) sequence identity to the nucleotide sequence of SEQ ID NO: 5273;
(xiii) an encoded light chain constant region, optionally wherein the nucleotide sequence encoding the light chain constant region comprises the nucleotide sequence of SEQ ID NO: 5221;
(ix) a rabbit globin polyA signal region, optionally wherein the rabbit globin polyA signal region comprises the nucleotide sequence of SEQ ID NO: 6590; and
(x) a 3′ AAV ITR, optionally wherein the 3′ AAV ITR comprises the nucleotide sequence of SEQ ID NO: 6561.
   601. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5261;
(viii) an encoded heavy chain constant region, optionally wherein the heavy chain constant region is encoded by the nucleotide sequence of SEQ ID NO: 5219;
(ix) an encoded furin cleavage site, optionally wherein the nucleotide sequence encoding the furin cleavage site comprises the nucleotide sequence of SEQ ID NO: 6557;
(x) an encoded T2A linker, optionally wherein the nucleotide sequence encoding the T2A linker comprises the nucleotide sequence of SEQ ID NO: 6558;
(xi) a signal sequence, optionally wherein the signal sequence comprises the nucleotide sequence of SEQ ID NO: 5159;
(xii) a transgene encoding a heavy chain variable region comprising a nucleotide sequence with at least 90% (e.g., at least about 95, 96, 97, 98, or 99%) sequence identity to the nucleotide sequence of SEQ ID NO: 6552, 6512, 6517, 6522, 6527, or 6532;
(xiii) an encoded glycine-serine linker, optionally wherein the glycine-serine linker is encoded by the nucleotide sequence of SEQ ID NO: 5347;
(xiv) a transgene encoding a light chain variable region (VL) comprising a nucleotide sequence with at least 90% (e.g., at least about 95, 96, 97, 98, or 99%) sequence identity to the nucleotide sequence of SEQ ID NO: 5265;
(xv) an encoded glycine-serine linker, optionally wherein the glycine-serine linker is encoded by SEQ ID NO: 5243;
(xvi) a transgene encoding a light chain variable region (VL) comprising a nucleotide sequence with at least 90% (e.g., at least about 95, 96, 97, 98, or 99%) sequence identity to the nucleotide sequence of SEQ ID NO: 5353;
(xvii) an encoded light chain constant region, optionally wherein the nucleotide sequence encoding the light chain constant region comprises a nucleotide sequence with at least 90% (e.g., at least about 95, 96, 97, 98, or 99%) sequence identity to the nucleotide sequence of SEQ ID NO: 5217; (xvii) a rabbit globin polyA signal region, optionally wherein the rabbit globin polyA signal region comprises the nucleotide sequence of SEQ ID NO: 6590; and
(xviii) a 3′ AAV ITR, optionally wherein the 3′ AAV ITR comprises the nucleotide sequence of SEQ ID NO: 6561.
   602. The AAV particle of any one of embodiments 547-583, wherein the genetic element comprises in 5′ to 3′ order:
(i) a 5′ adeno-associated (AAV) ITR, optionally wherein the 5′ AAV ITR comprises the nucleotide sequence of SEQ ID NO: 6559;
(ii) a CB promoter, optionally wherein the CB promoter comprises the nucleotide sequence of SEQ ID NO: 6566;
(iii) an ie exon 1 region, optionally wherein the ie exon 1 region comprises the nucleotide of SEQ ID NO: 6573;
(iv) an intron region, optionally wherein the intron region comprises the nucleotide sequence of SEQ ID NO: 6578 and SEQ ID NO: 6580;
(v) a human beta-globin exon region, optionally wherein the human beta-globin exon region comprises the nucleotide sequence of SEQ ID NO: 6576;
(vi) a signal sequence, optionally wherein the signal sequence comprises the nucleotide sequence of SEQ ID NO: 5157;
(vii) a transgene encoding a heavy chain variable region (VH) encoded by a nucleotide sequence comprising a nucleotide sequence with at least 90% (e.g., at least about 95, 96, 97, 98, or 99%) sequence identity to the nucleotide sequence of SEQ ID NO: 5002;
(viii) an encoded heavy chain constant region, optionally wherein the heavy chain constant region is encoded by the nucleotide sequence of SEQ ID NO: 5017;
(ix) an encoded furin cleavage site, optionally wherein the nucleotide sequence encoding the furin cleavage site comprises the nucleotide sequence of SEQ ID NO: 1724;
(x) an encoded T2A linker, optionally wherein the nucleotide sequence encoding the T2A linker comprises the nucleotide sequence of SEQ ID NO: 1726;
(xi) a signal sequence, optionally wherein the signal sequence comprises the nucleotide sequence of SEQ ID NO: 5159;
(xii) a transgene encoding a DARPIN comprising a nucleotide sequence with at least 90% (e.g., at least about 95, 96, 97, 98, or 99%) sequence identity to the nucleotide sequence of SEQ ID NO: 5371;
(xiii) an encoded glycine-serine linker, optionally wherein the glycine-serine linker is encoded by the nucleotide sequence of SEQ ID NO: 5347;
(xiv) a transgene encoding a light chain variable region (VL) comprising a nucleotide sequence with at least 90% (e.g., at least about 95, 96, 97, 98, or 99%) sequence identity to the nucleotide sequence of SEQ ID NO: 5005;
(xvi) an encoded light chain constant region, optionally wherein the nucleotide sequence encoding the light chain constant region comprises a nucleotide sequence with at least 90% (e.g., at least about 95, 96, 97, 98, or 99%) sequence identity to the nucleotide sequence of SEQ ID NO: 5007;
(xvii) a rabbit globin polyA signal region, optionally wherein the rabbit globin polyA signal region comprises the nucleotide sequence of SEQ ID NO: 6590; and
(xviii) a 3′ AAV ITR, optionally wherein the 3′ AAV ITR comprises the nucleotide sequence of SEQ ID NO: 6561.
   603. An isolated, e.g., recombinant, antibody molecule produced by the AAV particle of any one of the preceding embodiments.
   604. The AAV particle of any one of the preceding embodiments, wherein the genetic element further encodes:
(i) the AAV capsid polypeptide, e.g., the AAV capsid variant, e.g., a structural protein, wherein the capsid protein comprises a VP1 polypeptide, a VP2 polypeptide, and/or a VP3 polypeptide, optionally wherein the VP1 polypeptide, the VP2 polypeptide, and/or the VP3 polypeptide are encoded by at least one Cap gene; and/or
(ii) a Rep protein, e.g., a non-structural protein, wherein the Rep protein comprises a Rep78 protein, a Rep68, Rep52 protein, and/or a Rep40 protein (e.g., a Rep52 protein and/or Rep78 protein), optionally wherein the Rep78 protein, the Rep68 protein, the Rep52 protein, and/or the Rep40 protein are encoded by at least one Rep gene.
   605. The AAV particle of any one of embodiments 1-604, wherein the genetic element further comprises a nucleic acid encoding:
(i) the AAV capsid polypeptide, e.g., the AAV capsid variant, e.g., a structural protein, wherein the capsid protein comprises a VP1 polypeptide, a VP2 polypeptide, and/or a VP3 polypeptide, optionally wherein the VP1 polypeptide, the VP2 polypeptide, and/or the VP3 polypeptide are encoded by at least one Cap gene; and/or
(ii) a Rep protein, e.g., a non-structural protein, wherein the Rep protein comprises a Rep78 protein, a Rep68, Rep52 protein, and/or a Rep40 protein (e.g., a Rep52 protein and/or Rep78 protein), optionally wherein the Rep78 protein, the Rep68 protein, the Rep52 protein, and/or the Rep40 protein are encoded by at least one Rep gene.
   606. A cell, e.g., a host cell, comprising the AAV particle of any one of embodiments 1-202, optionally wherein the cell is a mammalian cell, an insect cell, or a bacterial cell.
   607. A method of making the AAV particle of any one of embodiments 1-605, the method comprising
(i) providing a host cell a the genetic element; and
(ii) incubating the host cell under conditions suitable to enclose the genetic element in an AAV capsid variant;
thereby making the isolated AAV particle.
   608. The method of embodiment 607, further comprising, prior to step (i), introducing a first nucleic acid molecule comprising the genetic element into the host cell.
   609. The method of embodiment 607 or 608, wherein the host cell comprises a second nucleic acid encoding an AAV capsid variant.
   610. The method of embodiment 609, further comprising introducing the second nucleic acid into the cell, optionally wherein the second nucleic acid molecule is introduced into the host cell prior to, concurrently with, or after the first nucleic acid molecule.
   611. A pharmaceutical composition comprising an AAV particle of any one of embodiments 1-605, and a pharmaceutically acceptable excipient.
   612. The pharmaceutical composition of embodiment 611, wherein the pharmaceutically acceptable excipient comprises a buffer, a gel, a hydrogel, or artificial cerebrospinal fluid.
   613. A method of delivering an exogenous antibody molecule that binds to HER2/neu, to a subject, comprising administering an effective amount of the AAV particle of any one of embodiments 1-605 or the pharmaceutical composition of embodiment 611 or 612.
   614. The method of embodiment 613, wherein:
(i) the subject has, has been diagnosed with having, or is at risk of having a disease associated with expression of HER2/neu; and/or
(ii) the subject has, has been diagnosed with having, or is at risk of having a cancer expressing HER2/neu.
   615. A method of treating a subject having or diagnosed with having cancer expressing HER2/neu, comprising administering to the subject an effective amount of the AAV particle of any one of embodiments 1-605 or the pharmaceutical composition of embodiment 611 or 612.
   616. The method of any one of embodiments 613-615, wherein the disease associated with HER2/neu expression is a HER2/neu-positive solid tumor.
   617. The method of embodiment 616, wherein the HER2/neu positive tumor is metastatic.
   618. The method of any one of embodiments 616 or 617, wherein the HER/neu positive cancer is breast cancer, gastric cancer, gastroesophageal junction cancer, colorectal cancer, lung cancer (e.g., non-small cell lung carcinoma), pancreatic cancer, bladder cancer, salivary duct cancer, ovarian cancer (e.g., epithelial ovarian cancer), endometrial cancer, prostate cancer, bone cancer and brain cancer.
   619. The method of any one of embodiments 616-618, wherein the HER2/neu positive cancer has metastasized to the central nervous system (CNS).
   620. The method of any one of embodiments 616-619, wherein the HER2/neu positive cancer is breast cancer.
   621. The method of any one of embodiments 613-620, wherein the subject is a human.
   622. The method of any one of embodiments 613-621, wherein the subject has previously undergone localized therapy for a HER2/neu-positive solid tumor.
   623. The method of any one of embodiments 613-622, wherein the subject has previously undergone surgical resection of a HER2/neu-positive solid tumor.
   624. The method of any one of embodiments 613-623, wherein the subject has previously undergone radiotherapy for a HER2/neu-positive solid tumor.
   625. The method of any one of embodiments 613-624, wherein the subject has previously undergone immunotherapy and/or chemotherapy for a HER2/neu-positive solid tumor.
   626. The method of any one of embodiments 613-625, wherein the subject has triple-negative breast cancer.
   627. The method of any one of embodiments 613-626, wherein the HER2/neu positive tumor is refractory.
   628. The method of any one of embodiments 613-627, wherein the method reduces or prevents metastases.
   629. The method of embodiment 628, wherein the metastases are brain metastases.
   630. The method of any one of embodiments 613-629, wherein the AAV particle is administered to the subject intramuscularly, intravenously, intratumorally, intracerebrally, intrathecally, intraarterially, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM).
   631. The method of any one of embodiments 613-630, wherein the AAV particle is administered to the subject intravenously.
   632. The method of any one of embodiments 613-630, wherein the AAV particle is administered to the subject intratumorally.
   633. The method of any one of embodiments 613-630, wherein the AAV particle is administered to the subject via intra-cisterna magna (ICM) injection.
   634. The method of any one of embodiments 613-630, wherein the AAV particle is administered prior to, concurrently with, or post a surgical resection of a HER2/neu-positive solid tumor.
   635. The method of any one of embodiments 613-630, wherein the AAV particle is administered to a site of surgical resection of a HER2/neu-positive solid tumor in the subject.
   636. The method of any one of embodiments 613-633, wherein the AAV particle is administered to the area around a site of surgical resection of a HER2/neu-positive solid tumor, e.g., the margins of the tumor, in the subject.
   637. The method of any one of embodiments 613-636, further comprising administration of an additional therapeutic agent and/or therapy suitable for treatment or prevention of a disorder associated with HER2/neu expression.
   638. The method of embodiment 637, wherein the additional therapeutic agent comprises:
(i) trastuzumab, pertuzumab, a chemotherapeutic agent, or a combination thereof;
(ii) trastuzumab emtansine; and/or
(iii) trastuzumab, tucatinib, capecitabine, Fam-trastuzumab deruxtecan-nxki, Lapatanib/capecitabine, Lapatanib, Margetuximab, a chemotherapeutic agent, Neratanib/capecitabine, or a combination thereof.
   639. The method of any one of embodiments 613-638, wherein the method reduces tumor cell proliferation.
   640. The method of embodiment 639, wherein the method induces an innate immune response to HER2/neu expressing tumor cells.
   641. The method of embodiment 640, wherein the innate immune response comprises an increase in CD45⁺ cells at the tumor site.
   642. The method of embodiment 640 or 641, wherein the innate immune response comprises an increase in the proportion of one or more of the following cell types at the tumor site
(i) active proliferating microglia;
(ii) dendritic cells (DC);
(iii) natural killer cells (NK); and/or
(iv) innate lymphocytic cells (ILC).
   643. The AAV particle of any one of embodiments 1-605, or the pharmaceutical composition of embodiment 611 or 612, for use in the manufacture of a medicament.
   644. The AAV particle of any one of embodiments 1-605, or the pharmaceutical composition of embodiment 611 or 612, for use in the treatment of a disease associated with expression of HER2/neu or a cancer expressing HER2/neu.
   645. Use of the AAV particle of any one of embodiments 1-605, or the pharmaceutical composition of embodiment 611 or 612, in the manufacture of a medicament.
   646. Use of the AAV particle of any one of embodiments 1-605, or the pharmaceutical composition of embodiment 611 or 612, in the manufacture of a medicament for treating a disease associated with expression of HER2/neu or a cancer expressing HER2/neu.

### BRIEF DESCRIPTION OF THE DRAWINGS

FIGS. 1A-1C are graphical representation of the data from the codon optimization of the anti-HER2 monospecific vectorized antibody genetic elements. FIGS. 1A and 1B show the expression comparison of HER-53 (SEQ ID NO: 5168), HER-75 (SEQ ID NO: 5190) and HER-77 (non-optimized) in HEK expi293 cells (adherent) 3 days post-transfection (FIG. 1A), and in HEK expi293 cells (suspension) at 2, 3, and 7 days post-transfection (FIG. 1B). The antibody concentration (μg/mL) is displayed on the Y-axis, and the antibody is indicated on the X-axis. Points on the columns indicate individual transfections. FIG. 1C shows the results from the codon optimization of the anti-HER2 monospecific vectorized antibody genetic elements in assays using HEK-Blue human TLR9 (hTLR9) cells. The Y-axis shows the OD630 value from the cell-based reporter gene assay, a surrogate for TLR9 activation level, and the synthesized DNA oligos from the codon optimized antibody coding sequence and the oligos from the corresponding non-codon optimized sequence is indicated on the X-axis. The codon optimization resulted in reduced NF-kB response to the DNA sequences in hTLR9 HEK Blue cells.

FIGS. 2A-2D are graphical representations of the BT-474 cell proliferation assay of the vectorized anti-HER2 antibodies HER-04 (SEQ ID NO: 5163) (FIG. 2A), HER-05 (SEQ ID NO: 5170) (FIG. 2B), HER-10 (SEQ ID NO: 5164) (FIG. 2C), and HER-15 (SEQ ID NO: 5165) (FIG. 2D), expressed by Expi293 cells. The relative viability is shown on the Y-axis as percent of human IgG1 isotype control antibody signal, and the antibody concentration is shown on the X-axis in μg/mL. BT-474 cell proliferation was measured using the CellTiter-Glo 2.0 reagent after four days of antibody treatment. Error bars: mean and standard deviation from four replicates.

FIG. 3 is a graphical representation of the BT-474 cell proliferation assay comparing the HER-53 (SEQ ID NO: 5168), human IgG1 isotype control antibody, HER-04 (SEQ ID NO: 5163), and HER-10 (SEQ ID NO: 5164) vectorized anti-HER2 antibodies expressed by Expi293 cells. The relative viability is shown on the Y-axis as percent of human IgG1 isotype control antibody signal, and the antibody concentration is shown on the X-axis in μg/mL. Similar cell growth inhibition was observed between the HER-53 antibody with an enhanced Fc region and HER-04. HER-10 demonstrated increased growth inhibition compared to HER-53 or HER-04. BT-474 cell proliferation was measured using the CellTiter-Glo 2.0 reagent after four days of antibody treatment. Error bars: mean and standard deviation from four replicates.

FIGS. 4A-4D are graphical representations of the MDA-MB-361-luc cell proliferation assay comparing the HER-04 (SEQ ID NO: 5163) (FIG. 4A), HER-10 (SEQ ID NO: 5164) (FIG. 4C) and HER-15 (SEQ ID NO: 5165) (FIG. 4D) vectorized anti-HER2 antibodies expressed by Expi293 cells with a recombinant reference antibody control (Creative Biolabs cat. #TAB-761) (FIG. 4B). The relative viability is shown on the Y-axis as percent of human IgG1 isotype control antibody signal, and the antibody concentration is shown on the X-axis in μg/mL. The vectorized anti-HER2 antibodies inhibited cell growth of MDA-MB-361-luc cells in a dose-dependent manner. Cell proliferation was measured using the CellTiter-Glo 2.0 reagent after 13 days of antibody treatment. Error bars: mean and standard deviation from three replicates.

FIGS. 5A-5G are graphical representations of the MDA-MB-361-luc cell proliferation assay with an expanded dose range comparing the human IgG1 isotype control antibody (FIG. 5A), HER-04 (SEQ ID NO: 5163) (FIG. 5B), HER-10 (SEQ ID NO: 5164) (FIG. 5D), HER-15 (SEQ ID NO: 5165) (FIG. 5E), and HER-53 (SEQ ID NO: 5168) (FIGS. 5F-5G), vectorized anti-HER2 antibodies expressed by Expi293 cells with a recombinant reference (naked)antibody control (Creative Biolabs cat. #TAB-761) (FIG. 5C). The relative viability is shown on the Y-axis as percent of human IgG1 isotype control antibody signal, and the antibody concentration is shown on the X-axis in μg/mL. The vectorized anti-HER2 antibodies inhibited cell growth of MDA-MB-361-luc cells in a dose-dependent manner. Cell proliferation was measured using the CellTiter-Glo 2.0 reagent after 13 days of antibody treatment. Error bars: mean and standard deviation from three replicates.

FIGS. 6A-6B are graphical representations of the MDA-MB-361-luc cell proliferation assay, with an expanded dose range, comparing the recombinant reference antibody control (Creative Biolabs cat. #TAB-761) (FIG. 6A), and HER-10 (SEQ ID NO: 5164) (FIG. 6B) vectorized anti-HER2 antibody expressed by Expi293 cells. The relative viability is shown on the Y-axis as percent of human IgG1 isotype control antibody signal, and the antibody concentration is shown on the X-axis in μg/mL. Error bars: mean and standard deviation from three replicates. The vectorized HER-10 antibody inhibited cell growth of MDA-MB-361-luc cells in a dose-dependent manner. Cell proliferation was measured using the CellTiter-Glo 2.0 reagent after 13 days of antibody treatment. Error bars: mean and standard deviation from three replicates.

FIGS. 7A-7B are graphical representations of the ADCC activity of human IgG1 isotype control antibody, HER-04 (SEQ ID NO: 5163), and HER-10 (SEQ ID NO: 5164) expressed by Expi293 cells in comparison to a reference antibody employing the high affinity (FIG. 7A) and low affinity (FIG. 7B) variants of the human FcgRIIIa receptor. The fold induction (relative to no antibody control) is shown on the Y-axis, and the antibody concentration is shown on the X-axis in μg/mL. HER-10 contains enhancements in the Fc region that promote ADCC activity. HER-10 ADCC activity is similar a reference anti-HER2 antibody, and is improved over HER-04 in assays employing both the high affinity and low affinity variants of the FcgRIIIa receptor. Error bars: mean and standard deviation from three replicates.

FIGS. 8A-8B are graphical representations of the ADCC activity of HER-53 (SEQ ID NO: 5168) in comparison to HER-10 (SEQ ID NO: 5164) and HER-04 (SEQ ID NO: 5163) vectorized anti-HER2 antibodies expressed by Expi293 cells, employing the high affinity (FIG. 8A) and low affinity (FIG. 8B) variants of the human FcgRIIIa receptor. The fold induction (relative to no antibody control) is shown on the Y-axis, and the antibody concentration is shown on the X-axis in μg/mL. ADCC activity is similar between HER-53 and HER-10 while both display increased activity over HER-04 in assays employing both the high affinity and low affinity variants of the FcgRIIIa receptor. Error bars: mean and standard deviation from three replicates.

FIGS. 9A-9D are graphical representations of the BT-474 cell (FIGS. 9A and 9B) and SK-BR-3 cell (FIGS. 9C and 9D) proliferation assays with tucatinib and HER-53 (SEQ ID NO: 5168), HER-75 (SEQ ID NO: 5190), human IgG1 isotype control antibody, HER-04 (SEQ ID NO: 5163), and HER-10 (SEQ ID NO: 5164) vectorized anti-HER2 antibodies expressed by Expi293 cells. The relative viability is shown on the Y-axis as percent of human IgG1 isotype control antibody signal, and the antibody concentration is shown on the X-axis in μg/mL. Similar cell growth inhibition was observed between the HER-53 and HER-04. Tucatinib-treated cells served as positive control for cell growth inhibition. Cell proliferation was measured using the CellTiter-Glo 2.0 reagent after four days. Error bars: mean and standard deviation from four replicates (FIG. 9A, tucatinib only: two replicates).

FIGS. 10A-10B are graphical representations of the BT-474 cell proliferation assay of HER-43 (SEQ ID NO: 5185) in comparison to HER-47 (SEQ ID NO: 5167) vectorized anti-HER2 antibodies expressed by Expi293 cells (FIG. 10A), and HER-46 (SEQ ID NO: 5186) in comparison to HER-47 (SEQ ID NO: 5167) vectorized anti-HER2 antibodies expressed by Expi293 cells (FIG. 10B). The relative viability is shown on the Y-axis as percent of human IgG1 isotype control signal, and the antibody concentration is shown on the X-axis in μg/mL. Similar cell growth inhibition was observed between HER-04 and engineered antibodies containing mutations that abrogate FcRn-binding (HER-43, HER-46, and HER-47). BT-474 cell proliferation was measured using the CellTiter-Glo 2.0 reagent after four days of antibody treatment. Error bars: mean and standard deviation from four replicates.

FIG. 11 is a graphical representation of the SK-BR-3 cell proliferation assay of the vectorized anti-HER2 antibody HER-57 (SEQ ID NO: 5187) in comparison to HER-04 (SEQ ID NO: 5163), human IgG1 isotype control antibody, HER-10 (SEQ ID NO: 5164) (expressed by Expi293 cells) and tucatinih. The relative viability is shown on the Y-axis as percent of human IgG1 isotype control antibody signal, and the antibody concentration is shown on the X-axis in μg/mL. SK-BR-3 cells were sensitive to cell growth inhibition by the HER-57 vectorized antibody. Tucatinib-treated cells served as positive control for cell growth inhibition. SK-BR-3 cell proliferation was measured using the CellTiter-Glo 2.0 reagent after four days. Error bars: mean and standard deviation from four replicates (tucatinib only: two replicates).

FIGS. 12A-12B are graphical representations of the SK-BR-3 cell proliferation assay (FIG. 12A) and the BT-474 cell proliferation assay (FIG. 12B) of the vectorized anti-HER2 antibody HER-53 (SEQ ID NO: 5168) in comparison to HER-75 (SEQ ID NO: 5190), human IgG1 isotype control antibody, HER-88 (SEQ ID NO: 6500) and tucatinib. The relative viability is shown on the Y-axis as percent of human IgG1 isotype control antibody signal, and the antibody concentration is shown on the X-axis in μg/mL. Tucatinib-treated cells served as positive control for cell growth inhibition.

FIGS. 13A-13C are images of mouse brain immunohistochemical staining depicting the tissue transduction distributions after IV administration of AAV vectors (4.0E11 vector genomes per mouse) carrying a CBA-promoter driven EGFP transgene in Fox Chase SCID CB17 mice (Charles River Labs, #236). After perfusion of animals, brains were dissected and then fixed in 10% Neutral Buffered Formalin. The fixed brain tissues were then paraffin embedded, sectioned, and stained with anti-EGFP and anti-human nucleoli antibodies. Immunohistochemistry (IHC) was performed with a chromogenic dye that correlated with the level of EGFP protein present in the tissue. IHC results are displayed on both ipsilateral (left side of diagram) and contralateral (right side of diagram) sagittal section in order of increasing brain distribution, where FIG. 13A is the VOY101 capsid, FIG. 13B is the VOY9P33 capsid. Tumor distinguished by human nucleoli staining is demarcated by black oval. FIG. 13C is the VOY9P39 capsid. Representative images are displayed for n=5 per cohort (AAV type). Scale bar indicates relative size in millimeters.

FIGS. 14A-14D are graphical representations of the mouse pharmacokinetics and pharmacodynamics of antibody expressed from IV administration of the HER10 construct, vectorized in the VOY9P39 capsid. Antibody concentration in mouse serum (FIG. 14A), CSF (FIG. 14B), and brain tissue (FIGS. 14C and 14D) after IV administration of AAV in Fox Chase SCID CB17 (Charles River Labs, #236). Human IgG1 was measured by an AlphaLISA assay. FIGS. 14A-14D X-axis indicates the days post treatment. In FIG. 14A the mean serum levels of human IgG1 (ug/ml) indicates increasing levels with time since injection of the AAV. In FIG. 14B the mean CSF levels remain constant (n=3 mice/cohort). In FIG. 14C, the mean brain levels also increase with time since AAV injection. In FIGS. 14A and 14B the Y-axis shows the concentration of hIgG1 (ug/ml), and in FIG. 14C the concentration of the hIgG1 is shown as a percent of total protein. In FIG. 14D, the number of viral vector genomes (VG) per diploid cell as measured by droplet digital PCR (ddPCR) indicates a decrease in mean VG with time since injection. In FIG. 14D the Y-axis depicts the number of viral vector genomes per diploid cell. The +/− standard error of the mean are depicted by whiskers in all panels.

FIGS. 15A-15D are graphical representation of the data from the prophylactic treatment of vectorized HER10. Prophylactic treatment 12 days prior to xenograft with IV administration of AAV particles (5.0E11 vector genomes per mouse) comprising a VOY9P39 capsid and either a genetic element comprising the HER-10 sequence (SEQ ID NO: 5164) (5.0E11 VG/mouse) or a sequence encoding an human IgG1 isotype control antibody to Fox Chase SCID CB17 mice (Charles River Labs, #236). FIG. 15A is a graph of mean bioluminescent imaging data (BLI) with photons/second displayed (Total Flux). Statistically significant reductions in tumor burden after HER10 treatment are denoted by stars, based on discovery determined using the two-stage linear step-up procedure of Benjamini, Krieger and Yekutieli, with Q=5% (Benjamini, Y., Krieger, A. M., & Yekutieli, D., 2006, Biometrika. 93, 491-507). Each row was analyzed individually, without assuming a consistent SD. Number of t tests: 3. Q values are adjusted for multiple comparison. Days 17; q=0.014, Day 24; q=0.031, Day 28; q=0.016 (n=10 mice/cohort). FIG. 15B shows plots of the mean values of IgG1 quantification at day 30 post xenograft from serum (ug/ml, P<0.001) and FIG. 15C is a plot of human IgG concentrations in the brain homogenate from mice, 30 days after xenotransplantation of MDA-MB-361-Luc tumor cells in the brain (% total protein, P<0.004). FIG. 15D is a plot of the mean values for vector genomes per diploid cell by group as measured from brain tissue homogenates by ddPCR quantification at day 30 post xenotransplantation of tumor cells. The +/− standard error of the mean are depicted by whiskers in all panels.

FIGS. 16A-16E are graphical representations of data generated from studies of Fox Chase SCID CB17 (Charles River Labs, #236) mice that underwent vectorized antibody treatment following tumor xenograft. Mice were xenotransplanted in the brain with MDA-MB-361-luc tumor cells 2 days prior to IV treatment (2.5e11 vector genomes per mouse). After allowing the engraftment of tumors for 2 days, mice were administered a genetic element comprising the HER-10 (FIGS. 16A-16C) or HER-53 (FIG. 16D-16E) sequence vectorized in a VOY9P39 capsid or a genetic element encoding a human IgG1 isotype control antibody vectorized in a VOY9P39 capsid. FIGS. 16A and 16D are graphs of the mean bioluminescent imaging data (BLI) for mice from each treatment group, with photons/second normalized to day 7 baseline reading. Statistically significant reductions in tumor burden after HER-10 and HER-53 treatment are denoted by stars, based on discovery determined using the two-stage linear step-up procedure of Benjamini, Krieger and Yekutieli, with Q=5% (Benjamini, Y., Krieger, A. M., & Yekutieli, D., 2006, Biometrika. 93, 491-507). Each row was analyzed individually, without assuming a consistent SD. FIG. 16A: number of t tests: 7; Q values are adjusted for multiple comparison; Days 21 & 28: q=0.001, Day 35; q=0.0015, Day 44; q=0.026, Day 49; q=0.0064 (n=5 mice/cohort). FIG. 16B is a plot of IgG1 concentrations in mouse serum as quantified by AlphaLISA at day 43 (q<0.0012. t-test), 63 (q=0.0019) and 99 (q=0.029) post AAV administration. Multiple t-tests, FDR(q) values reported. The +/− standard error of the mean are depicted by whiskers in all panels. FIGS. 16C and 16E are plots of the Kaplan-Meier curves depicting significant survival difference (P=0.0035; Log-rank (Mantel-Cox test) between HER-10 (FIG. 16C), HER-53 (FIG. 16E) and a human IgG1 isotype control antibody transduced mouse.

FIGS. 17A-17B are graphical representations of the data from the intracranial treatment of tumor xenografted mice with HER-10 (SEQ ID NO: 5164) or isotype control antibody vectorized in a VOY-101 capsid (6.0E10 vector genomes per mouse). FIG. 17A is a plot of the mean bioluminescent imaging data (BLI) for AAV treatment at Day 2 post xenograft, with photons/second normalized to day 7 baseline reading. FIG. 17B is a plot of the Kaplan-Meier curves depicting a survival difference between HER-10 and a human IgG1 isotype control antibody transduced mouse. HER-10 demonstrates persistent and significant tumor growth suppression as compared to vectorized human IgG1 isotype control treated CB17/SCID mice. Statistically significant reductions in tumor burden after HER-10 treatment are evident at indicated time points, based on discovery determined using multiple t-tests comparing human IgG1 isotype control vs HER-10. Each row was analyzed individually, without assuming a consistent SD. +/− standard error of the mean are depicted by whiskers in all panels.

FIG. 18 is an image of the immunohistochemical staining of anti mouse Cd11b (black) of MDA-MB-361 orthotopic xenografts in a mouse brain. The monocytes aggregate around the tumor periphery and infiltrate within the human tumor mass at 28 days post AAV9P39 treatment.

FIGS. 19A-19D are graphical representations of the cell proliferation assays of the vectorized anti-HER2 bispecific antibody HER-73 (SEQ ID NO: 5189) and HER-78 (SEQ ID NO: 5375) in comparison to human IgG1 isotype control antibody, HER-10 (SEQ ID NO: 5164) (expressed by Expi293 cells), ZW-25, and tucatinib in BT-474 cells (FIGS. 19A and 19C) and SK-BR-3 cells (FIGS. 19B and 19D). The relative viability is shown on the Y-axis as percent of human IgG1 isotype control antibody signal, and the antibody concentration is shown on the X-axis in μg/mL. The HER-73 and HER-78 antibodies demonstrated an increased cell growth inhibition of BT-474 cells in comparison to HER-10 or the ZW-25 bispecific antibody. Tucatinib-treated cells served as positive control for cell growth inhibition. BT-474 cell proliferation was measured using the CellTiter-Glo 2.0 reagent after four days. Similar to BT-474 cells, SK-BR-3 cells were sensitive to HER-73 and HER-78. Tucatinib-treated cells served as positive control for cell growth inhibition. SK-BR-3 cell proliferation was measured using the CellTiter-Glo 2.0 reagent after four days. Error bars: mean and standard deviation from four replicates (tucatinib only: two replicates).

FIG. 20A shows tumor burden as measured by total flux (photons/second) treated intravenously with AAV_TTM-002.HER-75 or AAV_TTM-002.Control particles over the days post xenograft (MDA-MB-361L cells) (n=5 mice per group, and p-values as measured by a 2 Way Anova Sidak's multiple comparison test). FIG. 20B shows the quantification of vectorized HER-75 antibodies or the isotype control (hIgG1 (μg/mL)) by alphaLISA in the CSF following treatment intravenous treatment with AAV_TTM-002.HER-75 or AAV_TTM-002.Control particles at day 53 post xenograft.

FIG. 20C provides the vector genome copies per diploid cell as measured by ddPCR in the brain following treatment intravenous treatment with AAV_TTM-002.HER-75 or AAV_TTM-002.Control particles at day 53 post xenograft. In FIGS. 20A-20C, the AAV particles were administered prophylactically to the mice 10 days prior to injection of the xenograft.

FIG. 21A shows tumor burden as measured by total flux (photons/second) treated intravenously with AAV_TTM-002.HER-75 or AAV_TTM-002.Control particles over the days post xenograft (MDA-MB-361L cells) (n=5 mice per group, and p-values as measured by a Dunnett's Multiple comparison test, 2 Way ANOVA) at the indicated doses. FIG. 21B shows the quantification of vectorized HER-103 antibodies or the isotype control (hIgG1 (μg/mL)) by alphaLISA in the serum following treatment intravenous treatment with AAV_TTM-002.HER-103 or AAV_TTM-002.Control particles at day 28 post treatment.

### DETAILED DESCRIPTION

Described herein, inter alia, are compositions comprising isolated, e.g., recombinant, viral particles, e.g., AAV particles, for delivery, e.g., vectorized delivery, of an antibody molecule and methods of making and using the same. In some embodiments, the antibody molecule is an antibody molecule that binds to HER2, e.g., an anti-HER2 antibody molecule described herein. Generally, the recombinant AAV particles will include a genetic element comprising a nucleotide sequence, e.g., encoding a transgene encoding an antibody molecule that binds to HER2.

Antibodies typically have short half-lives, presenting a challenge for antibody-based therapies. To achieve a sufficiently high concentration of an antibody for long lasting therapeutic effects, said antibody-based therapies are traditionally delivered by repeated administration, e.g. by multiple injections. These repeated dosing regimens can result in inconsistent levels of antibody throughout the treatment period, limited efficiency per administration, high cost of administration and consumption of the antibody. Hence, there is a need for improved methods of delivering antibodies and antibody-based therapeutics that increase duration and efficacy of the response and result in sustained, high levels of the therapeutic antibody.

Additionally, treatment modalities for brain and CNS diseases (e.g., HER2-positive metastatic cancer) are extremely limited due to the impermeability of the brain's blood vessels to most substances carried in the blood stream. The blood vessels of the brain, referred to collectively as the blood-brain barrier (BBB), are unique when compared to the blood vessels found in the periphery of the body. Tight apposition of BBB endothelial cells (EC) to neural cells like astrocytes, pericytes and neurons induces phenotypic features that contribute to the observed impermeability. Tight junctions between ECs comprising the BBB limit paracellular transport, while the lack of pinocytotic vesicles and fenestrae limit non-specific transcellular transport. These factors combine to restrict molecular flux from the blood to the brain to those molecules that are less than 500 daltons and also lipophilic. Thus, using the large mass transfer surface area of the bloodstream as a delivery vehicle is largely infeasible except in those circumstances where a drug with the desired pharmacological properties fortuitously possesses the size and lipophilicity attributes allowing it to pass freely through the blood vessel. Because of such restrictions, it has been estimated that greater than 98% of all small molecule pharmaceuticals and nearly 100% of the emerging class of protein (e.g., antibodies) and gene therapeutics do not cross the BBB.

Adeno-associated viral (AAV) vectors and particles are commonly used in gene therapy approaches as a result of a number of advantageous features. AAVs are typically non-replicating in infected cells, and therefore are generally not associated with disease. Further, AAVs may be introduced to a variety of host cells, do not integrate into the genome of the host cell, and are capable of infecting both quiescent and dividing cells. AAVs transduce non-replicating and long-lived cells in vivo, resulting in long term expression of the protein of interest. Further, AAVs can be manipulated with cellular and molecular biology techniques to produce non-toxic, isolated, recombinant, AAV particles comprising a payload that can be delivered to a target tissue or set of cells with limited or no side-effects. Without wishing to be bound by theory, it is believed in some embodiments, that expression vectors, e.g., an adeno-associated viral vector (AAVs) or AAV particle, e.g., an AAV particle described herein, can be used to administer and/or deliver antibody molecules, e.g., antibodies that bind to HER2, in order to achieve sustained, high concentrations, allowing for longer lasting efficacy, fewer dose treatments, and/or more consistent levels of the antibody throughout the treatment period.

Using a vectorized antibody delivery (VAD) approach of an anti-HER2 antibody described herein, an AAV particle is used as the delivery modality for a nucleotide sequence, e.g., an AAV vector, genetic element, or nucleic acid described herein, encoding a transgene encoding the anti-HER2 antibody molecule. In some embodiments, vectorized delivery of a functional anti-HER2 antibody molecule described herein, results in in vivo expression of the encoded antibody. In some embodiments, upon delivery of an AAV particle comprising genetic element comprising a nucleotide sequence encoding a transgene encoding an antibody molecule, the AAV particle enters the cell via endocytosis and is transported to the nucleus wherein the genetic element is released and converted into a double-stranded episomal molecule of DNA by the host cell. In some embodiments, the transcriptionally active episome results in the expression of encoded anti-HER2 antibodies (e.g., an anti-HER2 antibody molecule described herein) that is then secreted from the cell into the circulation.

Without wishing to be bound by theory, it is believed in some embodiments, that the use of an AAV particle or plurality of AAV particles for the vectorized delivery of an antibody molecule that binds to HER2 (e.g. an anti-HER2 antibody described herein) would lead to increased exposure in the central nervous system (CNS), and one-time administration would result in long-term, robust expression of anti-HER2 antibodies in the subject, e.g., a subject having or diagnosed with having a disease associated with over expression of HER2 (e.g., HER2+ metastatic cancer).

Without wishing to be bound by theory, it believed in some embodiments, that HER2+ tumors manifesting in the CNS exhibit sub-therapeutic thresholds of anti-HER2 antibody concentrations. In some embodiments, this sub-therapeutic thresholds of anti-HER2 antibody concentrations can result from blood-brain barrier that limits the entry of large biomolecules (e.g., antibodies) into the CNS. Additionally, rapid efflux of antibodies out of the CNS, has also been observed, e.g., by the HER2+ mouse brain xenograft studies with passive administration doses of trastuzumab that far exceed the clinical levels in addition to clinical trials of high-dose Trastuzumab+Pertuzumab combinations (Lin et al., Journal of Clinical Oncology 39(24): (2021)).

Without wishing to be bound by theory, it is believed that the anti-HER2 vectorized antibodies of the present disclosure generate durable expression of HER2-directed antibodies by both “factory” cells in the CNS (neurons, astrocytes, glial cells) in addition to the metastatic tumors that have infiltrated the CNS. In some embodiments, these cells transduced with AAV transgenes secrete antibody into the brain parenchyma, ISF, CSF, and tumor micro-environment. Without wishing to be bound by theory, it is believed in some embodiments, that this this can result in high levels of target engagement on HER2 amplified tumors leading to both disruption of aberrant HER-family receptor signaling and tumor killing by ADCC. In some embodiments, an AAV particle described herein encoding an anti-HER2 antibody molecule described herein may be administered through intravenous (IV), intra cisterna magna injection (ICM), and direct intra-tumoral injections. In some embodiments, each of the aforesaid routes of administration can complement the existing standard of care.

Also contemplated herein, inter alia, are compositions comprising an AAV capsid polypeptide, e.g., an AAV capsid variant, e.g., an AAV capsid variant described herein for delivery, e.g., vectorized delivery, of an anti-HER antibody molecule described herein, and methods of making and using the same. Generally, the AAV capsid variant has enhanced tropism for a cell or tissue, e.g., for the delivery of a payload to said cell or tissue, for example a CNS tissue or a CNS cell.

As demonstrated in the Examples herein below, certain AAV capsid variants described herein show multiple advantages over wild-type AAV9, including (i) increased penetrance through the blood brain barrier following intravenous administration, (ii) wider distribution throughout the multiple brain regions, e.g., frontal cortex, sensory cortex, motor cortex, putamen, thalamus, cerebellar cortex, dentate nucleus, caudate, and/or hippocampus, and/or (iii) elevated payload expression in multiple brain regions. Without wishing to be being bound by theory, it is believed that these advantages may be due, in part, to the dissemination of the AAV capsid variants through the brain vasculature. In some embodiments, the AAV capsids described herein enhance the delivery of a payload, e.g., an anti-HER-2 antibody molecule described herein, to multiple regions of the brain including for example, the frontal cortex, sensory cortex, motor cortex, putamen, midbrain thalamus, cerebellar cortex, dentate nucleus, caudate, and/or hippocampus. In some embodiments, the AAV capsids described herein enhance the expression of a payload, e.g., an anti-HER-2 antibody molecule described herein or mRNA encoding an anti-HER2 antibody molecule, to multiple cell types in the CNS, e.g., neurons and/or non-neuronal cells (e.g., oligodendrocytes, astrocytes, and/or glial cells). Without wishing to be bound by theory, an AAV particle comprising an AAV capsid polypeptide, e.g., an AAV capsid variant described herein, for the vectorized delivery of an antibody molecule that binds to HER2/neu described here will result in increased penetrance through the blood brain barrier and transduction in non-neuronal cells (e.g., glial cells, e.g., astrocytes and oligodendrocytes), e.g., following intravenous administration, and/or increased biodistribution of the antibody molecule that binds to HER2/neu in the central nervous system, e.g., the brain and the spinal cord. In some embodiments, an AAV capsid variant disclosed herein comprises a modification in loop IV of AAV9, e.g., at positions between 449-460, e.g., at position 454 and/or 455, numbered relative to SEQ ID NO: 138, 981, or 982. In some embodiments, loop (e.g., loop IV) is used interchangeably herein with the term variable region (e.g., variable region IV), or VR (e.g., VR-TV). In some embodiments loop IV comprises positions 449-475 (e.g., amino acids KTINGSGQNQQTLKFSVAGPSNMAVQG (SEQ ID NO: 7583)), numbered according to SEQ ID NO: 138. In some embodiments loop IV comprises positions 449-460 (e.g., amino acids KTINGSGQNQQT (SEQ ID NO: 7584)), numbered according to SEQ ID NO: 138. In some embodiments, loop IV or variable region IV (VR-IV) is as described in DiMattia et al. “Structural Insights into the Unique Properties of the Adeno-Associated Virus Serotype 9,” Journal of Virology, 12(86):6947-6958 (the contents of which are hereby incorporated by reference in their entirety), e.g., comprising positions 452-460 (e.g., NGSGQNQQT (SEQ ID NO: 7585)), numbered according to SEQ ID NO: 138.

### I. DEFINITIONS

Unless defined otherwise, all technical and scientific terms used herein have the same meaning as commonly understood by one of ordinary skill in the art to which the invention pertains.

Throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

The term “a” and “an” refers to one or to more than one (i.e., to at least one) of the grammatical object of the article. By way of example, “an element” means one element or more than one element.

The term “or” is used herein to mean, and is used interchangeably with the term “and/or”, unless context clearly indicates otherwise.

As used herein, the term “about” or “approximately” when referring to a measurable value such as an amount, a temporal duration, and the like, are meant to encompass variations of ±20% or in some instances ±10%, or in some instances ±5%, or in some instances ±1%, or in some instances ±0.1% from the specified value, as such variations are appropriate to perform the disclosed methods.

The term “AAV particle” or “AAV viral particle” refers to a particle or a virion comprising an AAV capsid, e.g., an AAV capsid variant, and a polynucleotide, e.g., a genetic element and/or a vector. In some embodiments, the genetic element of the AAV particle comprises at least one payload and at least one ITR region. In some embodiments, the AAV particle is capable of delivering a transgene encoding a payload to cells, typically, mammalian, e.g., human, cells. In some embodiments, the AAV particle may be produced recombinantly. In some embodiments, an AAV particle described herein may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted.

As used herein, the term “capsid” refers to the exterior, e.g., a protein shell, of a virus particle, e.g., an AAV particle, that is substantially (e.g., >50%, >>90%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

As used herein, the term “genetic element” refers to a nucleic acid sequence, generally in a particle, e.g., an AAV particle. The genetic element can be produced as naked DNA and optionally further assembled into a capsid. A particle, e.g., an AAV particle can insert its genetic element into a cell. For example, a payload of a genetic element described herein can be a polypeptide or a polynucleotide. In some embodiments, the genetic element comprises at least one inverted terminal repeat (ITR) and at least one payload. In some embodiments, the genetic element comprises a polynucleotide sequence encoding a payload flanked on one side by an ITR. In some embodiments, the genetic element comprises a polynucleotide sequence encoding a payload flanked on both sides by an ITR.

As used herein, a “viral genome” or “vector genome” is a polynucleotide comprising the genetic element. In some embodiments, the genetic element comprises at least one inverted terminal repeat (ITR) and at least one transgene encoding a payload, e.g., a payload region.

As used herein, a “transgene encoding a payload” or a “payload region” refers to a polynucleotide or polynucleotide region, e.g., within a viral genome, e.g., a genetic element, which encodes an expression product, e.g., a payload. In some embodiments, the payload is, or comprises, a polypeptide, e.g., an antibody molecule. In some embodiments, the payload comprises a transgene, a polynucleotide encoding a polypeptide or multi-polypeptide, e.g., antibody molecule, or a modulatory nucleic acid or regulatory nucleic acid.

As used herein, a “vector” is any molecule or moiety which transports, transduces or otherwise acts as a carrier of a heterologous molecule. In some embodiments, vectors may be plasmids. Vectors of the present disclosure may be produced recombinantly. The heterologous molecule may be a polynucleotide and/or a polypeptide.

As used herein, the term “administered in combination” or “combined administration” means that two (or more) agents are delivered to a subject during the course of the subject's affliction with the disorder, for example, the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, for example, an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

As used herein, the term “amelioration” or “ameliorating” refers to a decreasing, e.g., lessening, of the severity of at least one indicator or parameter of a condition or disease. In some embodiments, amelioration of an indicator of a condition or disease in a subject results from treating the subject with a treatment described herein, as compared to a second subject that has not received the treatment. In some embodiments, the indicator or parameter of a condition or disease comprises a sign and/or symptom of the disorder. For example, in the context of neurodegeneration disorder, amelioration includes the reduction of neuron loss.

As used herein, the term “antibody” or “antibody molecule” refers to a protein comprising at least one immunoglobulin variable domain sequence. Antibodies, for example, can be polyclonal or monoclonal, multiple or single chain, or intact immunoglobulins, and may be derived from natural sources or from recombinant sources. Antibodies can be tetramers of immunoglobulin molecules. In some embodiments, an antibody molecule comprises a full-length antibody, or a full-length immunoglobulin chain. In some embodiments, an antibody molecule comprises an antigen binding or functional fragment of a full-length antibody, or a full-length immunoglobulin chain. In some embodiments, the term antibody includes functional fragments thereof. In some embodiments, constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

As used herein, the term “antibody fragment” refers to at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, for example, two, Fab fragments linked by a disulfide bridge at the hinge region, or two or more, for example, two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The terms “complementarity determining region” or “CDR,” as used herein, refer to the sequences of amino acids within antibody variable regions which confer antigen specificity and binding affinity. For example, in general, there are three CDRs in each heavy chain variable region (for example, HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, for example, with respect to the N-terminal and C-terminal ends of the polypeptide, the scFv may comprise VL-linker-VH or may comprise VH-linker-VL. In some embodiments, the scFv may comprise the structure of NH2-VL-linker-VH-COOH or NH2-VH-linker-VL-COOH.

As used herein, an “immunoglobulin variable domain sequence” or “variable domain” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to a polypeptide, or an epitope thereof. With respect to proteins (or protein mimetics), the antigen-binding site typically includes one or more loops (of at least, e.g., four amino acids or amino acid mimics) that form an interface that binds to a polypeptide. Typically, the antigen-binding site of an antibody molecule includes at least one or two CDRs and/or hypervariable loops, or more typically at least three, four, five or six CDRs and/or hypervariable loops.

As used herein, the term “epitope” refers to the moieties of an antigen that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

The term “antibody heavy chain,” or “heavy chain” refers to the larger of the two types of polypeptide chains present in antibody molecules in their naturally occurring conformations, and which normally determines the class to which the antibody belongs.

The term “antibody light chain,” or “light chain” refers to the smaller of the two types of polypeptide chains present in antibody molecules in their naturally occurring conformations. Kappa (κ) and lambda (λ) light chains refer to the two major antibody light chain isotypes.

As used herein, the terms “multibody” or “multispecific antibody” refer to an antibody comprising a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap or substantially overlap. In some embodiments, the first and second epitopes do not overlap or do not substantially overlap. In some embodiments, the first and second epitopes are on different antigens, e.g., different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or tetraspecific antibody molecule.

As used herein, the term “bispecific antibody” refers to an antibody that has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap or substantially overlap. In some embodiments, the first and second epitopes do not overlap or do not substantially overlap (e.g., a biparatopic antibody). In some embodiments, the first and second epitopes are on different antigens, e.g., different proteins (or different subunits of a multimeric protein). In some embodiments, a bispecific antibody is able to bind two different antigens simultaneously or sequentially. Methods for making bispecific antibodies are well known in the art. Various formats for combining two antibodies are also known in the art. Forms of bispecific antibodies of the invention include, but are not limited to, a diabody, a single-chain diabody, Fab dimerization (Fab-Fab), Fab-scFv, and a tandem antibody, as known to those of skill in the art.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods). “Humanized” forms of non-human (for example, murine) antibodies are chimeric immunoglobulins, immunoglobulin chains or fragments thereof (such as Fv. Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” as used herein refers to an immunoglobulin, such as an antibody or antibody fragment, where the whole molecule is of human origin or consists of an amino acid sequence identical to a human form of the antibody or immunoglobulin.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule. In some embodiments, the domains in the CAR polypeptide construct are in the same polypeptide chain, for example, comprise a chimeric fusion protein. In some embodiments, the domains in the CAR polypeptide construct are not contiguous with each other, for example, are in different polypeptide chains.

As used herein, the terms “associated with,” “conjugated,” “linked,” “attached,” “coupled,” and “tethered,” when used with respect to two or more moieties, means that the moieties are associated or connected, e.g., physically or chemically, with one another, either directly or via one or more additional moieties that serves as a linking agent, to form a structure that is sufficiently stable so that the moieties remain physically associated under the conditions in which the structure is used, e.g., physiological conditions. In some embodiments, the two or more moieties are covalently or non-covalently linked, coupled, or attached. In some embodiments, an association is through direct covalent chemical bonding. In other embodiments, the association is through ionic or hydrogen bonding or a hybridization based connectivity sufficiently stable such that the associated or linked entities remain physically associated.

As used herein, the term “complementary” when used to describe a first nucleotide sequence in relation to a second nucleotide sequence, refers to the ability of an oligonucleotide or polynucleotide comprising the first nucleotide sequence to hybridize and form base pairs, e.g., a duplex, with an oligonucleotide or polynucleotide comprising the second nucleotide sequence. In some embodiments, base pairs are typically formed by hydrogen bonds between nucleotide units in antiparallel polynucleotide strands. In some embodiments, complementary polynucleotide or oligonucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. The term “complementary” as used herein can encompass fully complementary, partially complementary, or substantially complementary.

“Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand. Where a first sequence is referred to as “substantially complementary” with respect to a second sequence herein, the two sequences can be fully complementary or they may form one or more, but generally not more than 5, 4, 3, or 2 mismatched or non-complimentary base pairs upon hybridization for a duplex, while still retaining the ability to hybridize under the conditions most relevant to their ultimate application. In some embodiments, two strands in which some but not all nucleotide units can base pair are considered substantially complementary or to have less than perfect complementarity. For example, for two 20-mers, if only two base pairs on each strand can base pair with each other, the polynucleotide strands exhibit 10% complementarity. In the same example, if 18 base pairs on each strand can base pair with each other, the polynucleotide strands exhibit 90% complementarity. In some embodiments, a siRNA (e.g., the antisense strand) that is substantially complementary to a desired target mRNA, has a sequence (e.g., the antisense strand) which is sufficient to bind the desired target mRNA, and to trigger the RNA silencing of the target mRNA.

As used herein, “control elements”, “regulatory control elements”, or “regulatory sequences” refers to elements used for expression of a gene or gene product. In some embodiments, these “control elements”, “regulatory control elements”, or “regulatory sequences” comprise promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

As used herein, the term “encapsulate” means to enclose, surround or encase. As an example, a capsid protein, e.g., an AAV capsid variant, often encapsulates a genetic element. In some embodiments, encapsulate within a capsid, e.g., an AAV capsid variant, encompasses 100% coverage by a capsid, as well as less than 100% coverage, e.g., 95% or less. For example, gaps or discontinuities may be present in the capsid so long as the genetic element is retained in the capsid, e.g., prior to entry into a cell.

As used herein, the term “effective amount” which can be used interchangeably herein, refer to an amount of a compound, formulation, material, or composition, as described herein to achieve a particular biological result. In some embodiments, an effective amount is a “therapeutically effective amount.” In some embodiments, the effective amount of an agent is that amount sufficient to effect beneficial or desired results, for example, clinical results. For example, in the context of administering an agent that treats cancer, an effective amount of an agent is, for example, an amount sufficient to achieve treatment, as defined herein, of cancer, as compared to the response obtained without administration of the agent.

As used herein, “expression” refers to transcription and/or translation of a particular nucleotide sequence. In some embodiments, expression comprises one or more of the following: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein the term “heterologous” region or element (e.g., a nucleic acid sequence or an amino acid sequence), refers to a region or element that would not be considered a homologous region or element. In some embodiments, the heterologous region or element when used with respect to another region or element, refers to regions or elements that would not naturally be found together, e.g., in a wild-type virus, e.g., an AAV. In some embodiments, a heterologous nucleic acid sequence may be present in the same nucleic acid as a naturally occurring nucleic acid sequence (e.g., a sequence that is naturally occurring in the AAV). In some embodiments, a heterologous region or element is exogenous relative to an AAV from which other (e.g., the remainder of) elements/regions of the AAV particle are based.

As used herein the term “homologous region” refers to a region which is similar in position, structure, evolution origin, character, form or function.

As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

As used herein, the term “identity” refers to the subunit sequence identity between two polymeric molecules, e.g., between two nucleic acid molecules (e.g. two DNA molecules and/or two RNA molecules) and/or between two polypeptide molecules. When a subunit position in both of the two molecules is occupied by the same monomeric subunit; for example, if a position in each of two DNA molecules is occupied by adenine, then they are identical at that position. The identity between two sequences is a direct function of the number of matching positions; for example, if half (for example, five positions in a polymer ten subunits in length) of the positions in two sequences are homologous, the two sequences are 50% identical; if 90% of the positions (for example, 9 of 10), are matched, the two sequences are 90% identical.

Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, at least 99%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part 1, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein

As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

As used herein “linker” refers to a molecule or group of molecules which connects two molecules, such as to link a variable heavy and a variable light chain in the context of an scFv or an antibody. In some embodiments, the linker is a nucleic acid sequence connecting two nucleic acid sequences encoding two different polypeptides. In some embodiments, the linker may or may not be translated. In some embodiments, the linker is a cleavable linker. In some embodiments, the linker is a polypeptide linker, e.g., a flexible polypeptide linker, that comprises amino acids such as glycine or serine residues used alone or in combination.

The term “Fyn SH3-derived polypeptide”, used interchangeably herein with the term “Fynomer”, refers to a non-immunoglobulin-derived binding polypeptide (e.g. a so-called scaffold) derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12). FynomAbs are fusion proteins of an antibody and a Fyn SH3-derived binding protein (Brack et al. (2014), Mol Cancer Ther 13(8):2030-2039).

As used herein, the phrase “operably linked” refers to a functional connection between two or more molecules, constructs, transcripts, entities, moieties or the like. In some embodiments, operably linked refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, a “microRNA (miRNA) binding site” or a “miR binding site” comprises a nucleic acid sequence (whether RNA or DNA, e.g., differ by “U” of RNA or “T” in DNA) that is capable of binding, or binds, in whole or in part to a microRNA (miR) through complete or partial hybridization. Typically, such binding occurs between the miR and the miR binding site in the reverse complement orientation. In some embodiments, the miR binding site is transcribed from the AAV genetic element encoding the miR binding site.

In some embodiments, a miR binding site may be encoded or transcribed in series. Such a “miR binding site series” or “miR BSs” may include two or more miR binding sites having the same or different nucleic acid sequence.

As used herein, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

As used herein, the term “polypeptide” refers to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures. In some embodiments, the polypeptide is greater than 50 amino acids in length.

As used herein, “peptide” is less than or equal to 50 amino acids long, e.g., about 5, 10, 15, 20, 25, 30, 35, 40, 45, or 50 amino acids long.

The term “polypeptide variant” refers to molecules which differ in their amino acid sequence from a native or reference sequence. In some embodiments, the amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

As used herein the term “conservative sequence modification” refers to the modification of an amino acid that does not significantly affect or alter the characteristics of the protein, e.g., the binding characteristics of an antibody or antibody fragment. Such conservative modifications include substitutions, additions, or deletions. Modifications can be introduced into a sequence described herein, e.g., an antibody or antibody fragment described herein, by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (for example, lysine, arginine, histidine), acidic side chains (for example, aspartic acid, glutamic acid), uncharged polar side chains (for example, glycine, asparagine, glutamine, serine, threonine, tyrosine, cysteine, tryptophan), nonpolar side chains (for example, alanine, valine, leucine, isoleucine, proline, phenylalanine, methionine), beta-branched side chains (for example, threonine, valine, isoleucine) and aromatic side chains (for example, tyrosine, phenylalanine, tryptophan, histidine).

“Insertional variants” when referring to proteins are those with one or more amino acids inserted, e.g., immediately adjacent or subsequent, to a position in an amino acid sequence. “Immediately adjacent” or “immediately subsequent” to an amino acid means connected to either the alpha-carboxy or alpha-amino functional group of the amino acid.

“Deletional variants” when referring to proteins, are those with one or more amino acids in deleted from a reference protein.

The term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

The term “functional variant” refers to a polypeptide variant or a polynucleotide variant that has at least one activity of the reference sequence.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “preventing,” “prevent,” and “prevention” refer to an action that occurs before the subject begins to suffer from the condition, or relapse of the condition. Prevention need not result in a complete prevention of the condition; partial prevention or reduction of the condition or a symptom of the condition, or reduction of the risk of developing the condition, is encompassed by this term.

As used herein, a “prophylaxis” means the prevention of or protective treatment for a disease or disease state.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

As used herein, a “self-complementary viral particle” is a particle comprised of at least two components, a capsid and a polynucleotide sequence encoding a self-complementary genome (e.g., a genetic element) enclosed within the capsid. As used herein, the phrase “signal sequence” refers to a sequence which can direct the transport or localization of a protein.

As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, an animal refers to any member of the animal kingdom. In some embodiments, animal refers to a human at any stage of development. In some embodiments, animal refers to a non-human animal at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone. In some embodiments, the subject is a patient.

An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” is generally used herein to refer to compositions, e.g., compositions described herein, that are not naturally occurring.

As used herein, “naturally occurring” or “wild-type” refers to a substance or entity that has not been altered, e.g., structurally altered, or removed from the natural state, e.g., removed from at least some of component with which it is associated in the natural state. In some embodiments, a naturally occurring when referring to sequence refers to a sequence identical to a wild-type sequence or a naturally occurring variant thereof.

As used herein, “transfection,” “transformed,” or “transduced” refers to a process by which an exogenous nucleic acid is transferred or introduced into a host cell. A “transfected” or “transformed” or “transduced” cell is one which has been transfected, transformed, or transduced with exogenous nucleic acid. The cell includes the primary subject cell and its progeny.

As used herein, the terms “treat,” “treatment,” and “treating” refer to partially or completely alleviating, ameliorating, improving, relieving, delaying onset of, inhibiting progression of, reducing duration of, reducing severity of, and/or reducing incidence of one or more symptoms or features (preferably, one or more discernable symptoms) of an infection, disease, disorder, and/or condition, resulting from administration of one or more therapies (for example one or more therapeutic agents such as an AAV particle of the invention). In some embodiments, the terms “treat”, “treatment” and “treating” refer to the amelioration of at least one measurable physical parameter of a disorder. In other embodiments, the terms “treat”, “treatment” and “treating” refer to the inhibition of the progression of a disorder, either physically by, for example, stabilization of a discernible symptom, physiologically by, for example, stabilization of a physical parameter, or both. In other embodiments the terms “treat”, “treatment” and “treating” refer to the reduction or stabilization of a symptom of the disorder. For example, “treating” a cancer may refer to inhibiting survival, growth, and/or spread of a tumor or a reduction or stabilization of tumor size or cancerous cell count. In some embodiments, treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “Her-2,” “ErbB2,” “c-Erb-B2,” “HER2,” “Her2,” and “neu” are used interchangeably and refer to native HER2, and allelic variants thereof, as described, for example, in Semba et al., 1985, P.N.A.S. USA 82:6497-650 and Yamamoto et al., 1986, Nature 319:230-234 and Genebank accession number X03363. Unless indicated otherwise, the terms “HER2,” “ErbB2,” “c-Erb-B2,” “HER2,” and “Her2” when used herein refer to the human protein. The gene encoding Her2 is referred to herein as “ErbB2.”

As used herein, the term “HER2/ErbB2 status” refers to assessment of expression of HER2/ErbB2 in a patient, or patient's cells (e.g., cancer cells) as a biomarker, and the status typically is reported as “HER2/ErbB2 positive” when the biomarker is present in overabundance as compared to a normal healthy non-cancer breast tissue sample or “HER2/ErbB2 negative” when the biomarker is present at a level no greater than a normal healthy non-cancer breast tissue sample as determined by an IHC stain test of a fixed tissue sample. Various methods are known in the art for assessing HER2/ErbB2 status, typically focusing on the amount of the receptor (IHC), or mRNA levels (qPCR), or gene copy number (FISH), that is expressed by a patient's cells to thereby diagnose a patient as HER2/ErbB positive (when this receptor is overexpressed or amplified in the patient's cells) or HER2/ErbB negative (when this receptor is not overexpressed or not amplified on patient's cells). Overexpression and amplification are terms of art describing levels elevated above those found in similar tissue from a normal disease-free individual.

As used herein, the term “chemotherapy” or “chemotherapeutic agent” refers to treatment with a cytostatic or cytotoxic agent (i.e., a compound) to reduce or eliminate the growth or proliferation of undesirable cells, for example cancer cells. Thus, as used herein, “chemotherapy” or “chemotherapeutic agent” refers to a cytotoxic or cytostatic agent used to treat a proliferative disorder, for example cancer.

As used herein, the term “targeted pathway drug,” “pathway drug,” or “targeted drug,” refers to any molecule or antibody with therapeutic capacity designed to bind to a specific biomolecule (e.g., protein) involved in a disease process, thereby regulating its activity.

As used herein, the terms “HER2 therapy” or “HER2-targeted therapy” refer to treatments using one or more therapeutic agents that are designed to specifically target the HER2 molecule and/or signaling pathway(s), including but not limited to, for example antibodies and small molecules that target the HER2 molecule and/or signaling pathway(s). Such HER2 therapies may also target other members of the HER family, for example therapies that target both HER1 and HER2, HER1, HER2, and HER4, or HER3 alone.

As used herein, the term “CNS neoplasms” includes primary or metastatic cancers, which may be located in the brain (intracranial), meninges (connective tissue layer covering brain and spinal cord), or spinal cord.

As used herein, the term “progression-free survival (PFS)” refers to the time from treatment to first disease progression or death. For example, it is the time that the subject remains alive, without return of the cancer, e.g., for a defined period of time such as about 1 month, 1.2 months, 2 months, 2.4 months, 2.9 months, 3 months, 3.5 months, 4, months, 6 months, 7 months, 8 months, 9 months, 1 year, about 2 years, about 3 years, etc., from initiation of treatment or from initial diagnosis.

As used herein, the term “overall survival (OS)” refers to the subject remaining alive for a defined period of time, such as about 1 year, about 2 years, about 3 years, about 4 years, about 5 years, about 10 years, etc., from initiation of treatment or from initial diagnosis.

### II. COMPOSITIONS

According to the present disclosure, compositions for delivering functional anti-HER2 antibodies by adeno-associated virus particles (AAVs) are provided. In some embodiments, an AAV particle, e.g., an AAV particle as described herein, or plurality of particles, may be provided, e.g., delivered, via any of several routes of administration, to a cell, tissue, organ, or organism, in vivo, ex vivo, or in vitro.

In some embodiments, AAV particles, nucleic acids, e.g., nucleic acid molecules encoding an antibody molecule, and/or payloads, e.g., an antibody molecule, and methods of using and making the same are described in WO2017189963, the contents of which are herein incorporated by reference in their entirety.

In some embodiments, the nucleic acid sequences, genetic elements, AAV vectors, and polypeptides disclosed herein may be engineered to contain modular elements and/or sequence motifs assembled to enable expression of an antibody molecule or fragment thereof, e.g., an antibody molecule described herein. In some embodiments, the genetic element comprises a nucleotide sequence encoding a transgene encoding an antibody molecule (e.g., an antibody molecule described herein). In some embodiments, the nucleic acid sequence encodes an antibody molecule comprising one or more of the CDRs (e.g., heavy chain and/or light chain CDRs) of an antibody molecule, a variable heavy (VH) chain region and/or variable light (VL) chain region, a heavy and/or light chain constant region, a heavy and/or light chain, or a combination thereof. In some embodiments, the nucleic acid sequence encoding the antibody molecule may also encode a linker, e.g., such that the VH/heavy chain and the VL/light chain of the encoded antibody molecule are connected via a linker. In some embodiments, the order of expression, structural position, or concatemer count (e.g., the VH, VL, heavy chain, light chain, and/or linker) may be different within or among genetic element sequences. In some embodiments, the identity, position, and number of linkers expressed by a genetic element described herein may vary. In some embodiments, the genetic element may further comprise an internal repeat (ITR) sequence, promoter region, an intron region, an exon region, a Kozak sequence, an enhancer, a polyadenylation sequence, or combination thereof.

In some embodiments, the present disclosure provides methods for delivering an antibody molecule (e.g., an anti-HER2 antibody described herein) and/or a nucleic acid sequence encoding an antibody molecule (e.g., an anti-HER2 antibody described herein) comprised within the genetic element comprised within a recombinant, AAV particle (e.g., an AAV particle described herein) to a cell, tissue, organ, or subject.

### Adeno-Associated Viruses (AAVs) and AAV Particles

In some embodiments, AAV are used as a biological tool due to a relatively simple structure, their ability to infect a wide range of cells (including quiescent and dividing cells) without integration into the host genome and without replicating, and their relatively benign immunogenic profile. In some embodiments, the genome, e.g., genetic element, of the virus may be manipulated to contain a minimum of components for the assembly of a functional recombinant virus, or viral particle, which is loaded with or engineered to target a particular tissue and express or deliver a desired payload, e.g., an antibody molecule (e.g., an anti-HER2 antibody molecule).

In some embodiments, the AAV, e.g., naturally occurring (e.g., wild-type) AAV or a recombinant AAV, comprises a genetic element which is a linear, single-stranded nucleic acid molecule, e.g., DNA (ssDNA). In some embodiments, the genetic element, e.g., of a naturally occurring (e.g., wild-type) AAV, is approximately 5,000 nucleotides (nt) in length. In some embodiments, inverted terminal repeats (ITRs) traditionally cap the viral genome at both the 5′ and the 3′ end, providing origins of replication for the viral genome. In some embodiments, an AAV genetic element comprises two ITR sequences. In some embodiments, the ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In some embodiments, the AAV particle, e.g., an AAV particle (e.g., ssAAVs) described herein comprises a viral genome, e.g., genetic element and/or AAV vector, that is self-complementary (scAAV). In some embodiments, the ssAAV comprises nucleic acid molecules, e.g., DNA strands, that anneal together to form double stranded DNA. In some embodiments, a scAAV allows for rapid expression in a transduced cell as it bypasses second strand synthesis.

In some embodiments, the AAV genetic element further comprises nucleotide sequences for two open reading frames, one for the four non-structural Rep proteins (e.g., Rep78, Rep68, Rep52, and/or Rep40 (e.g., Rep52 and Rep78), encoded by Rep genes) and one for the three capsid, or structural, proteins (e.g., VP1, VP2, VP3, encoded by capsid genes or Cap genes). The Rep proteins are used for replication and packaging, while the capsid proteins are assembled to create the protein shell of the AAV particle, or AAV capsid. In some embodiments, alternative splicing and alternate initiation codons and promoters result in the generation of four different Rep proteins from a single open reading frame and the generation of three capsid proteins from a single open reading frame. For example, in some embodiments, for the AAV9/hu.14 serotype (SEQ ID NO: 138 and 137; e.g., as described in U.S. Pat. No. 7,906,111, the contents of which are herein incorporated by reference in their entirety), VP1 refers to amino acids 1-736, VP2 refers to amino acids 138-736, and VP3 refers to amino acids 203-736. In some embodiments, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleotide sequence encoding these proteins can be similarly described. In some embodiments, the three capsid proteins assemble to create the AAV capsid protein. In some embodiments, the AAV capsid protein typically comprises a molar ratio of 1:1:10 of VP1:VP2:VP3.

In some embodiments, a genetic element of a wild-type, e.g., naturally occurring, AAV can be modified to replace the rep/cap sequences with a nucleic acid comprising a transgene encoding a payload, e.g., an antibody molecule, wherein the genetic element comprises at least one ITR region. In some embodiments, the genetic element of a recombinant AAV comprises two ITR regions, e.g., a 5′ITR or a 3′ITR. In some embodiments, the rep/cap sequences can be provided in trans during production to generate AAV particles. In some embodiments, the genetic element of an AAV is comprised in an AAV vector, which further encodes a capsid protein e.g., a structural protein, wherein the capsid protein comprises a VP1 polypeptide, a VP2 polypeptide, and/or a VP3 polypeptide; and/or a Rep protein, e.g., a non-structural protein, wherein the Rep protein comprises a Rep78 protein, a Rep68, Rep52 protein, and/or a Rep40 protein (e.g., a Rep25 protein and/or a Rep78 protein).

In some embodiments, in addition to the genetic element comprising a nucleic acid encoding a transgene encoding a payload (e.g., an antibody molecule, e.g., an anti-HER2 antibody molecule), an AAV particle, e.g., an AAV particle described herein, may comprise the genetic element, in whole or in part, of any naturally occurring and/or recombinant AAV serotype nucleotide sequence or variant. In some embodiments, AAV variants may have sequences of significant homology at the nucleic acid (genetic element or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, AAV particles of the present disclosure are recombinant AAV particles which are replication defective and lacking the nucleotide sequences encoding functional Rep and Cap proteins. In some embodiments, these defective AAV particles may lack most or all parental coding sequences and carry only one or two AAV ITR sequences and the nucleic acid of interest for delivery to a cell, a tissue, an organ, or an organism.

In some embodiments, the genetic element or the AAV vector of the AAV particles described herein comprise at least one control element which provides for the replication, transcription, and translation of a coding sequence encoded therein. In some embodiments, a sufficient number of control elements are present such that the coding sequence of the transgene encoded by the genetic element is capable of being replicated, transcribed, and/or translated in a host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

In some embodiments, the recombinant AAV particles of the present disclosure are capable of providing, e.g., delivering, a transgene to a mammalian cell. In some embodiments, the recombinant AAV particles of the present disclosure are capable of vectorized delivery of an antibody molecule (e.g., an anti-HER2 antibody molecule) or fragment thereof.

In some embodiments, the AAV particles, vectors, genetic elements, and/or nucleic acids of the present disclosure may be produced recombinantly and may be based on adeno-associated virus (AAV) parent or reference sequences. Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety). In some embodiments, the AAV particles described herein may be modified to enhance the efficiency of delivery, e.g., delivery of a transgene encoding a payload, e.g., an antibody molecule. Without wishing to be bound by theory, it is believed in some embodiments, that a modified, e.g., recombinant, AAV particle can be packaged efficiently and successfully infect target cells at high frequency and with minimal toxicity. In some embodiments, the capsid protein of the AAV particles is engineered according to the methods described in US Publication Number US20130195801, the contents of which are incorporated herein by reference in their entirety.

### AAV Capsids and Variants Thereof

In some embodiments, an AAV particle, e.g., an AAV particle for the vectorized delivery of an antibody molecule described herein (e.g., an HER-2 antibody molecule), may comprise an AAV capsid polypeptide, e.g., an AAV capsid variant. In some embodiments, the AAV capsid polypeptide, e.g., an AAV capsid variant comprises a VOY101 capsid polypeptide, a VOY9P39 capsid polypeptide, a VOY9P33 capsid polypeptide, a AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 1, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide comprises any one of the nucleotide sequence in Table 1, or a nucleotide sequence substantially identical (e.g., having at least 70%, 75%, 80%, 85%, 90%, 92%, 95%, 97%, 98%, or 99% sequence identity) thereto.

TABLE 1
Exemplary full length capsid sequences

| | SEQ | |
|---|---|---|
| | ID | |
| Description | NO: | Sequence Information |
| VOY101 | 1 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9/hu.14 | 138 | MAADGYLPDWLEDNLSEGIREWWALKPGAPQPKANQQHQDNARGLVLPGYKYLGPGNGL |
| WT (amino | | DKGEPVNAADAAALEHDKAYDQQLKAGDNPYLKYNHADAEFQERLKEDTSFGGNLGRAV |
| acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 137 | ATGGCTGCCGATGGTTATCTTCCAGATTGGCTCGAGGACAACCTTAGTGAAGGAATTCG |
| WT (DNA) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| VOY9P39 | 12 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSEGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTG |
| | | STTGWAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| VOY9P39 | 13 | ATGGCTGCCGATGGTTATCTTCCAGattqqcTCGAGGACAACCTTAGTGAAGGAATTCG |
| (DNA) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGACGGAACAGGA |
| | | AGCACAACAGGATGGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGgTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| VOY9P33 | 14 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTG |
| | | QVTGWAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| VOY9P33 | 15 | ATGGCTGCCGATGGTTATCTTCCAGattggcTCGAGGACAACCTTAGTGAAGGAATTCG |
| (DNA) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGACGGAACAGGA |
| | | CAAGTCACAGGATGGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGgTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |

In some embodiments, the AAV capsid polypeptide, e.g., the AAV capsid variant, comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications but no more than 30, 20, or 10 modifications, e.g., substitutions, relative to the amino acid sequence of SEQ ID NO: 138. In some embodiments, the AAV capsid polypeptide, e.g., the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide, e.g., the AAV capsid variant, comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the AAV capsid polypeptide, e.g., the AAV capsid variant, comprises substitution at position K449, e.g., a K449R substitution, numbered according to SEQ ID NO: 138.

In some embodiments, the AAV capsid polypeptide, e.g., the AAV capsid variant, comprises the amino acid sequence of SEQ ID NO: 12 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications but no more than 30, 20, or 10 modifications, e.g., substitutions, relative to the amino acid sequence of SEQ ID NO: 12. In some embodiments, the AAV capsid polypeptide, e.g., the AAV capsid variant, comprises an amino acid sequence encoded by the nucleotide sequence of SEQ ID NO: 13 or a nucleotide sequence substantially identical (e.g., having at least 70%, 75%, 80%, 85%, 90%, 92%, 95%, 97%, 98%, or 99% sequence identity) thereto.

In some embodiments, the AAV capsid variant, comprises the amino acid sequence of SEQ ID NO: 14 or an amino acid sequence substantially identical (e.g., having at least 70%, 75%, 80%, 85%, 90%, 92%, 95%, 97%, 98%, or 99% sequence identity) thereto. In some embodiments the AAV capsid polypeptide, e.g., the AAV capsid variant, comprises an amino acid sequence comprising at least one, two, or three modifications but no more than 30, 20, or 10 modifications, e.g., substitutions, relative to the amino acid sequence of SEQ ID NO: 14. In some embodiments, the AAV capsid polypeptide, e.g., the AAV capsid variant, comprises an amino acid sequence encoded by the nucleotide sequence of SEQ ID NO: 15 or a nucleotide sequence substantially identical (e.g., having at least 70%, 75%, 80%, 85%, 90%, 92%, 95%, 97%, 98%, or 99% sequence identity) thereto.

In some embodiments, the AAV capsid variant, comprises immediately subsequent to position 448, 449, 452, 453, 455, numbered relative to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12-15, PHP.N, PHP.B, or an AAV serotype as provided in Table 6 of WO 2021/230987 (the contents of which are hereby incorporated by reference in their entirety)), at least 3, 4, 5, 6, 7, 8, 9, 10, 11, 12, or 13 consecutive amino acids of any of amino acid sequence provided in Tables 1A, 2A, 2B, 9-11, 15, and 16. In some embodiments, the at least 3, 4, 5, 6, 7, 8, 9, 10, 11, 12, or 13 consecutive amino acids of any of amino acid sequence provided in Tables 1A, 2A, 2B, 9-11, 15, and 16 replaces at least one, two, three, four, five, six, seven, eight, nine, ten, or all of positions K449, T450, I451, N452, G453, 5454, G455, Q456, N457, Q458, and/or Q459, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12, SEQ ID NO: 14, PHP.N, PHP.B, or an AAV serotype as provided in Table 6 of WO 2021/230987 (the contents of which are hereby incorporated by reference in their entirety). In some embodiments, the at least 3, 4, 5, 6, 7, 8, 9, 10, 11, 12, or 13 consecutive amino acids of any of amino acid sequence provided in Tables 1A, 2A, 2B, 9-11, 15, and 16 replaces positions S454, G455, or both positions S454 and G455, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12, SEQ ID NO: 14, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at one, two, three, four, five, six, seven, eight, nine or all of positions T450, I451, N452, G453, S454, G455, Q456, N457, Q458, and/or Q459, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12, SEQ ID NO: 14, PHP.N, PHP.B, or an AAV serotype as provided in Table 6 of WO 2021/230987 (the contents of which are hereby incorporated by reference in their entirety). In some embodiments, the AAV capsid variant comprises an amino acid other than the wild-type, e.g., native, amino acid, at position S454, G455, or both positions S454 and G455, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12, or SEQ ID NO: 14, PHP.N, PHP.B, or an AAV serotype as provided in Table 6 of WO 2021/230987 (the contents of which are hereby incorporated by reference in their entirety)). In some embodiments, the AAV capsid variant comprises a modification, e.g., substitution, at one, two, three, four, five, six, seven, eight, nine, ten or all of positions K449, T450, I451, N452, G453, S454, G455, Q456, N457, Q458, and/or Q459, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12, SEQ ID NO: 14, PHP.N, PHP.B, or an AAV serotype as provided in Table 6 of WO 2021/230987 (the contents of which are hereby incorporated by reference in their entirety). In some embodiments, the AAV capsid variant comprises a modification, e.g., substitution, at position S454, G455, or both positions S454 and G455, numbered according to SEQ ID NO: 138 or corresponding to equivalent positions in any other AAV serotype (e.g., AAV1, AAV2, AAV3, AAV3b, AAV4, AAV5, AAV6, AAV7, AAV8, AAVrh8, AAVrh10, AAVrh32.33, AAVrh74, SEQ ID NO: 1, SEQ ID NO: 12, SEQ ID NO: 14, PHP.N, PHP.B, or an AAV serotype as provided in Table 6 of WO 2021/230987 (the contents of which are hereby incorporated by reference in their entirety).

In some embodiments, an AAV capsid variant, e.g., an AAV capsid variant described herein (e.g., an AAV capsid variant comprising a peptide described herein). In some embodiments, an AAV capsid variant comprises a peptide as set forth in any of Tables 1A, 2A, 2B3, 9-11, 15, or 16.

TABLE 1A
Exemplary Peptide Sequences

| | SEQ | | SEQ | | SEQ | | SEQ |
|---|---|---|---|---|---|---|---|
| Peptide | ID | Peptide | ID | Peptide | ID | Peptide | ID |
| Sequence | NO: | Sequence | NO: | Sequence | NO: | Sequence | NO: |
| GSGSPHSKAQN | 200 | GSLHHDNHGQN | 385 | GSVFGVPSG | 570 | GSIAMTSHG | 755 |
| QQT | | QQT | | QNQQT | | QNQQT | |
| GHDSPHKSGQN | 201 | GIMARDSSGQN | 386 | GSGLPDRNL | 571 | GSPGVSPSG | 756 |
| QQT | | QQT | | QNQQT | | QNQQT | |
| GSGSPHARMQN | 202 | GVVHITNSGQN | 387 | GSGTHNSAIQ | 572 | GSGQNQQTG | 757 |
| QQT | | QQT | | NQQT | | SSSRV | |
| GSGSPHVKSQN | 203 | GSGQNQHSAPF | 388 | GSGMIIASMQ | 573 | GSGQHLPLL | 758 |
| QQT | | NQT | | NQQT | | GNQQT | |
| GQDSPHKSGQN | 204 | GSGQTSGLKQN | 389 | GGITWTDSG | 574 | GSDHSHRGG | 759 |
| QQT | | QQT | | QNQQT | | QNQQT | |
| GSGSPHASRQN | 205 | GSGQNQQTSLS | 390 | GSGQNQQAS | 575 | GSGIVTKLG | 760 |
| QQT | | NTA | | GRQQT | | QNQQT | |
| GSGSPHASRQN | 206 | GSGQNQAVHNK | 391 | GSGQNQQPH | 576 | GSGQDVTKT | 761 |
| KQT | | SQT | | LKSLT | | GNQQT | |
| GSGSPHVKIQNQ | 207 | GVHTHLPSGQN | 392 | GPPQHMTSG | 577 | GSGQNQQSH | 762 |
| QT | | QQT | | QNQQT | | GRIGT | |
| GSGSPHSKAKN | 208 | GHLTMHNSGQN | 393 | GSGQNQQAS | 578 | GSGQNQQIN | 763 |
| QQT | | HQT | | LPSRT | | HRSPT | |
| GSGSPHKKNQN | 209 | GSGSSSRPYQNQ | 394 | GSGQIVSTQT | 579 | GSGDDSRVG | 764 |
| QQT | | QT | | NQQT | | QNQQT | |
| GSGSPHVRMQN | 210 | GILLATPSGQNQ | 395 | GSGKGHSAG | 580 | GSGQSTLKRI | 765 |
| QQT | | QT | | QNQQT | | NQQT | |
| GSGSPHASRQK | 211 | GSGQNAGSIPN | 396 | GSGQNTRLQ | 581 | GSGSQHSKA | 766 |
| QQT | | QQT | | LGQQT | | QNQQT | |
| GHSSPHRSGQN | 212 | GSRDGHTVGQN | 397 | GSVGSRPVG | 582 | GSGQNQQHA | 767 |
| QQT | | QQT | | QNQQT | | SSNNT | |
| GMRTYHLSGQN | 213 | GSLLISTSGQNQ | 398 | GSSHTLALG | 583 | GSRTYQVSG | 768 |
| QQT | | QT | | QNQQT | | QNQQT | |
| GSGSPHTRGQN | 214 | GSGAMPSHGQN | 399 | GMYEYSQSG | 584 | GSGQNQGLL | 769 |
| QQT | | QQT | | QNQQT | | SSPQT | |
| GSGIIPVSSQNQ | 215 | GALVSPISGQNQ | 400 | GNGQNQQHS | 585 | GSGGGLQHN | 770 |
| QT | | QT | | ILHGT | | QNQQT | |
| GSEYGHKSGQN | 216 | GSLSSHGVGQN | 401 | GSGYNQPHL | 586 | GSGQNQQTT | 771 |
| QQT | | QQT | | QNQQT | | AATRM | |
| GRGQNVSSVHR | 217 | GSGQNQQASLA | 402 | GPLVNASSG | 587 | GSGQNQRAS | 772 |
| QQT | | MRT | | QNQQT | | ILVQT | |
| GSSHRFYGGQN | 218 | GPGLGSHSGQN | 403 | GSGQNQQVL | 588 | GSGQNLGLL | 773 |
| QQT | | QQT | | TTART | | GAQQT | |
| GYFVAAWSGQN | 219 | GHDSQHKSGQN | 404 | GSGQNQHSV | 589 | GSLDLGRSG | 774 |
| QQT | | QQT | | HNDQT | | QNQQT | |
| GSVLHSHAAQN | 220 | GSGLTLSATQN | 405 | GAGLIMHSG | 590 | GNSQVKVSG | 775 |
| QQT | | QQT | | QNQQT | | QNQQT | |
| GSGDLVVSTQN | 221 | GSGQVVAHVGN | 406 | GMGRHSASG | 591 | GSSGSHQYG | 776 |
| QQT | | QQT | | QNQQT | | QNQQT | |
| GSYGMAASGQN | 222 | GSGLRTMTTQN | 407 | GSHSQSGHG | 592 | GSGQNQQQR | 777 |
| QQT | | QQT | | QNQQT | | DGTLT | |
| GLNHFGASGQN | 223 | GSGQVGRLLQN | 408 | GSSTTIVSGQ | 593 | GRGQHVSVA | 778 |
| QQT | | QQT | | NHQT | | NNQQT | |
| GSTGSHSAGQN | 224 | GSGQLSHQSVN | 409 | GRHLVTASG | 594 | GDSSSRISGQ | 779 |
| QHT | | QQT | | QNQQT | | NQQT | |
| GLAGHTVSGQN | 225 | GSGDRYQTLQN | 410 | GSGQNQQHA | 595 | GSGQNQQHS | 780 |
| QQT | | QQT | | NLNQT | | LSSQT | |
| GIILGASSGQNQ | 226 | GSGQNQQLKSS | 411 | GSGSTHSKA | 596 | GSLMDVHRG | 781 |
| QT | | AQT | | QNQQT | | QNQQT | |
| GSGVSTYNIQNQ | 227 | GSGQNQYSIPVA | 412 | GSGQNKQML | 597 | GSIQYQSSGQ | 782 |
| QT | | QT | | SGNTT | | NQQT | |
| GSLVSVQTGQN | 228 | GSGERLHLTQN | 413 | GSGQVHNPT | 598 | GLGSKNPSG | 783 |
| QQT | | QQT | | QNQQT | | QNQQT | |
| GQSSPHRSGQN | 229 | GSGHNQQVRTA | 414 | GSGQNQQIP | 599 | GSGQLVLTL | 784 |
| QQT | | PNT | | HVHQT | | QNQQT | |
| GREYGHKSGQN | 230 | GGLSHVMSGQN | 415 | GSLHAGLSG | 600 | GSGQNQQTS | 785 |
| QQT | | QQT | | QNQQT | | QPLPG | |
| GHTLTLSSGQN | 231 | GSGQSHRDVLN | 416 | GPAQHGTSG | 601 | GSGQNQQNL | 786 |
| QQT | | QQT | | QNQQT | | GKLNT | |
| GSITLIPSGQNQ | 232 | GSGQNLAGRMD | 417 | GEKAVTSSG | 602 | GTTAHQPSG | 787 |
| QT | | QQT | | QNQQT | | QNQQT | |
| GSNGFTALGQN | 233 | GSGQNQQTNRG | 418 | GSGQNQQTM | 603 | GSGQNRAQI | 788 |
| QQT | | NPM | | ANGQR | | GTQQT | |
| GSGHSSHSVQN | 234 | GSGQSYQRDHN | 419 | GSGSPHSKD | 604 | GSGQYVHVS | 789 |
| QQT | | QQT | | QNQQT | | SNQQT | |
| GSGIPQRSGKNQ | 235 | GSLLSAGMGQN | 420 | GSFSMGYGG | 605 | GSGQNQQTA | 790 |
| QT | | QHT | | QNQQT | | HAFNI | |
| GSGDTLHMLQN | 236 | GSGQNQQTAIY | 421 | GSGTHLVSL | 606 | GSGQNQRTM | 791 |
| QQT | | RNI | | QNQQT | | VATQT | |
| GERHTVLSGQN | 237 | GSGQNQQTSGT | 422 | GSGQMQPHV | 607 | GSGQNPIRG | 792 |
| QQT | | TNC | | QNQQT | | AMQQT | |
| GSGMPQSHIQN | 238 | GMTSHSVSGQN | 423 | GSGQNQQVA | 608 | GSGYVITGS | 793 |
| QQT | | QQT | | GLNNT | | QNQQT | |
| GSGQLSGIGGNQ | 239 | GSSQSTGYQPN | 424 | GSSQNQQHD | 609 | GRGPKQSNI | 794 |
| QT | | QQT | | MRLRT | | QNQQT | |
| GSGQNRKPASF | 240 | GSLKPTTLGQN | 425 | GPASLPISGQ | 610 | GSGQNQQT | 795 |
| AQT | | QQT | | NQQT | | MLGKPC | |
| GSGSVSQLGQN | 241 | GRMFSLGSGQN | 426 | GSGQNQQPP | 611 | GSGQNQQVG | 796 |
| QQT | | QQT | | LATRT | | STVRT | |
| GSDFLGTHGQN | 242 | GSGQNQQTALG | 427 | GSSRVPVSG | 612 | GNVTTQKSG | 797 |
| QQT | | VKC | | QNQQT | | QNQQT | |
| GQIVQNPSGQN | 243 | GAMVSHSSGQN | 428 | GSGQNQQTN | 613 | GSGNPVSHL | 798 |
| QQT | | QQT | | LGHTT | | QNQQT | |
| GSGTQIPSQQNQ | 244 | GSGQNQQRNSD | 429 | GSGQNQQLV | 614 | GSLSHMESG | 799 |
| QT | | SVT | | SRVQT | | QNQQT | |
| GSGQNQQSARE | 245 | GSGQSMTLHLN | 430 | GPNSYPVSG | 615 | GRAPTNLSG | 800 |
| GLT | | QQT | | QKQQT | | QNQQT | |
| GSGLGMSTGQN | 246 | GSGQVHQAEVN | 431 | GHAHYQASG | 616 | GSGQNQQTV | 801 |
| QQT | | QQT | | QNQQT | | MTARA | |
| GSGLPVLSGQN | 247 | GSGQNQSQNHL | 432 | GSGQALLST | 617 | GSGMPASRL | 802 |
| QQT | | QQT | | GNQQT | | QNQQT | |
| GSGHSIRTDQNQ | 248 | GSLLTTASGQN | 433 | GSGQLPRQM | 618 | GVVRNHQSG | 803 |
| QT | | QQT | | TNQQT | | QNQQT | |
| GSGQSVQTVVN | 249 | GSGLIRTAAQNQ | 434 | GSGFPKSTEQ | 619 | GSGQNQHSV | 804 |
| QQT | | QT | | NQQT | | QVRQT | |
| GSGQNRAQSRF | 250 | GSGQNQQTVSR | 435 | GSRETSLSGQ | 620 | GSGQNTGHL | 805 |
| QQT | | QST | | NQQT | | TMQQT | |
| GGGDLGRSSQN | 251 | GSGQYANHGIN | 436 | GSGQNQQGT | 621 | GSGQNQQYA | 806 |
| QQT | | QQT | | GVSHT | | GKILT | |
| GGGTKMDSGQN | 252 | GSRSTGPSGQN | 437 | GSRTVPVYG | 622 | GSGNPHVRN | 807 |
| QQT | | QQT | | QNQQT | | QNQQT | |
| GSGSPHPSRQNQ | 253 | GRGVQQKLQQN | 438 | GSNAQSAHG | 623 | GSGQNGGSS | 808 |
| QT | | QQT | | QNQQT | | NRQQT | |
| GSGQITNAGMN | 254 | GSGQNQQVHLS | 439 | GAFHLAASG | 624 | GSGQRLSQG | 809 |
| QQT | | TGT | | QNQQT | | VNHQT | |
| GGRNGHTVGQN | 255 | GSGQNQQLSAK | 440 | GSGQYRSSS | 625 | GSGQNAHAK | 810 |
| QQT | | SST | | DNQQT | | EGQQT | |
| GSGFGPQTGQN | 256 | GSGYKAARPQN | 441 | GSGQVYISTP | 626 | GSSPAPNSG | 811 |
| QQT | | QQT | | NQQT | | QNQQT | |
| GRTDSHTSGQN | 257 | GSAGISPSGQNQ | 442 | GSGVSTQLL | 627 | GLAHKTSSG | 812 |
| QQT | | QT | | QNQQT | | QNQQT | |
| GYEVLGSSGQN | 258 | GSGQNRAHAFL | 443 | GSGQLGLSV | 628 | GSGQNQQTP | 813 |
| QQT | | QQT | | TNQQT | | GAHKT | |
| GSVHLSVTGQN | 259 | GSGLSGITMQN | 444 | GSGSNMRLS | 629 | GSGQNQQSL | 814 |
| QQT | | QQT | | QNQQT | | SGSFT | |
| GFMSYKGSGQN | 260 | GPGSAHSSGQN | 445 | GSGQNLHSG | 630 | GSGQNQQST | 815 |
| QQT | | QQT | | LPQQT | | GTSRT | |
| GNIAGSVSGQN | 261 | GSSHTQALGQN | 446 | GSSHTLALG | 631 | GSGQNQQTV | 816 |
| QQT | | QQT | | QNKQT | | QSNLV | |
| GSGSHRDVSQN | 262 | GSGVHGVSSQN | 447 | GSGQNQHSL | 632 | GSGQNQQLG | 817 |
| QQT | | QQT | | PAHRT | | SRQCT | |
| GGLGSMSSGQN | 263 | GSSGRDMGGQN | 448 | GSGQNQGTV | 633 | GSGQNQYLR | 818 |
| QQT | | QQT | | YPNQT | | LELQT | |
| GSGHLPQSAQN | 264 | GERAFPTSGQN | 449 | GSGQNQQPS | 634 | GSGQNQQTS | 819 |
| QQT | | QQT | | LRQST | | PRLQT | |
| GGVLVGGSGQN | 265 | GGRIVSLSGQNQ | 450 | GSGQNARLK | 635 | GSGQNQQTT | 820 |
| QQT | | QT | | DNQQT | | SSNMT | |
| GTHPYTSSGQN | 266 | GSGQNSYSHTS | 451 | GHAGSTGSG | 636 | GTASTYNSG | 821 |
| QQT | | QQT | | QNQQT | | QNQQT | |
| GSGQNQQLKEN | 267 | GLGYPGSSGQN | 452 | GSGQALSSSG | 637 | GSGQNQQT | 822 |
| RST | | QQT | | NQQT | | MPQHKI | |
| GSGQNQQTSPH | 268 | GSGPQSHTGQN | 453 | GSGASESHR | 638 | GSGQSHLHT | 823 |
| NHT | | QQT | | QNQQT | | GNQQT | |
| GSGTLYPQSQN | 269 | GSGQNQQLSRD | 454 | GVGVITSSGQ | 639 | GVKGVGHSG | 824 |
| QQT | | AST | | NQQT | | QNQQT | |
| GSGQNQQSNWI | 270 | GSGQILHSVPNQ | 455 | GSLYGQSLG | 640 | GSGKVTKQS | 825 |
| TKT | | QT | | QNQQT | | QNQQT | |
| GSGYTSLFLQNQ | 271 | GSGFHTDSRQN | 456 | GSGQMSDVH | 641 | GSGQNQQTA | 826 |
| QT | | QQT | | GNQQT | | LEKSL | |
| GSGVMTHVLQN | 272 | GSGQSHSLATN | 457 | GSGQNQQHS | 642 | GSGYKDTYG | 827 |
| QQT | | QQT | | SKATT | | QNQQT | |
| GSVSDVRAGQN | 273 | GSGQNQQTLSK | 458 | GSGQNQQTS | 643 | GSGQNQQSG | 828 |
| QQT | | PWT | | VSQQT | | TFLST | |
| GSGQSHMATLN | 274 | GSGHAAISQQN | 459 | GSGQKMWK | 644 | GSGQNTGQH | 829 |
| QQT | | QQT | | LDNQQT | | MMQQT | |
| GSGLSVHLAQN | 275 | GSGQNQQQIGG | 460 | GSGQNVSMQ | 645 | GSGKNQQRP | 830 |
| QQT | | NST | | VNQQT | | GLDQT | |
| GSGLSHATQQN | 276 | GGGPMAGSGQN | 461 | GSGQNQRAT | 646 | GSGQSREISL | 831 |
| QQT | | QQT | | LSNQT | | NQQT | |
| GSGLSVQSGQN | 277 | GMRMEYQSGQ | 462 | GSGQASSKS | 647 | GTPTSPSSGQ | 832 |
| QQT | | NQQT | | ANQQT | | NQQT | |
| GSGHMTYREKN | 278 | GSGQNQQGTLL | 463 | GSGKNQTPIP | 648 | GKPAGGLSG | 833 |
| QQT | | HQT | | KGQT | | QNQQT | |
| GSKGVPTPGQN | 279 | GSGQNQRSSGG | 464 | GSGQNQQTR | 649 | GSGQNHRSA | 834 |
| QQT | | VQT | | QEGST | | DMQQT | |
| GSGLLPLSSQNQ | 280 | GSGQNQRGALA | 465 | GASSLATSG | 650 | GSGQNQQTL | 835 |
| QT | | TQT | | QNQQT | | PSLSL | |
| GNGLYAVSGQN | 281 | GSGTVHAATQN | 466 | GSGQRGSLT | 651 | GSPYMGATG | 836 |
| QQT | | QQT | | ENQQT | | QNQQT | |
| GFNGSPSSGQNQ | 282 | GSRMTQQFGQN | 467 | GSEQTRQRG | 652 | GSGHAKAVG | 837 |
| QT | | QQT | | QNQQT | | QNQQT | |
| GSGQIRHSDQN | 283 | GSSSPGASGQN | 468 | GSGQNQQTL | 653 | GHMKGVTS | 838 |
| QQT | | QQT | | TASKE | | GQNQQT | |
| GGQVAPSSGQN | 284 | GHPSPHVSGQN | 469 | GSGTSGKTG | 654 | GSGQNQKIL | 839 |
| QQT | | QQT | | KNQQT | | TLDQT | |
| GSFSMHTHGQN | 285 | GSGSHHASRQN | 470 | GQLVTFTSG | 655 | GSGQNQQTK | 840 |
| QQT | | QQT | | QNQQT | | VGHSA | |
| GSGQNQQVIQG | 286 | GAVGHSYSGQN | 471 | GSGQNQQSA | 656 | GIARTTISGQ | 841 |
| SNT | | QQT | | NKILT | | NQQT | |
| GRVLHSHAGQN | 287 | GSRSQYDIGQN | 472 | GSGQNQQHH | 657 | GSGQNQQTS | 842 |
| QQT | | QQT | | SSHTT | | VGFRT | |
| GSGQNQQTSLQ | 288 | GSGQGPQERGN | 473 | GSGQNQKG | 658 | GSGQNQQT | 843 |
| DQT | | QQT | | MQPNQT | | MIANIR | |
| GSGLGRAPVQN | 289 | GSIAHVGTGQN | 474 | GSGQLVSGL | 659 | GDMTRSSSG | 844 |
| QQT | | QQT | | YNQQT | | QNQQT | |
| GNGFSSASGQN | 290 | GSGQNQQKQNH | 475 | GSSVGVPSG | 660 | GSGHMSDLR | 845 |
| QQT | | GNT | | QNQQT | | QNQQT | |
| GSGQMASRESN | 291 | GSGQNQQALGS | 476 | GSGQNQQW | 661 | GRGAVMAS | 846 |
| QQT | | QRT | | DSRRQT | | GQNQQT | |
| GPGLPNHSGQN | 292 | GSGAITHMPQN | 477 | GSEQTRQSG | 662 | GSGQNQQLS | 847 |
| QQT | | QQT | | QNQQT | | GKSVT | |
| GNIQWQGSGQN | 293 | GSGQRNPLLLN | 478 | GSGIGSHIPQ | 663 | GSHTLVVSG | 848 |
| QQT | | QQT | | NQQT | | QNQQT | |
| GMSAHMSSGQN | 294 | GSSGIPVSHQNQ | 479 | GSGQNQRLH | 664 | GSGPWSAGL | 849 |
| QQT | | QT | | GVDQT | | QNQQT | |
| GHSFVNRSGQN | 295 | GVHSLTPSGQN | 480 | GEVSRVLSG | 665 | GSGQHSPHA | 850 |
| QQT | | QQT | | QNQQT | | LNQQT | |
| GRAVMDHSGQ | 296 | GVIVLHGSGQN | 481 | GSGQNQQKV | 666 | GSGQNQQPN | 851 |
| NQQT | | QQT | | SPLLT | | SGSMT | |
| GALTVMQSGQN | 297 | GGTRVVDSGQN | 482 | GSGLALERS | 667 | GSGLAHLGG | 852 |
| QQT | | QQT | | QNQQT | | QNQQT | |
| GSGQRSPVLPNQ | 298 | GSGGVTYQSQN | 483 | GPDRIGSSGQ | 668 | GSSVRYEPK | 853 |
| QT | | QQT | | NQQT | | QNQQT | |
| GSGQNGHLSLK | 299 | GSGQNQAGHGP | 484 | GSGQNQDHQ | 669 | GSGQNQQAR | 854 |
| QQT | | GQT | | NKQQT | | PLELT | |
| GSLPRGTSDQN | 300 | GSGQLVTSGPN | 485 | GSGQNQQTA | 670 | GSGQPRSTGI | 855 |
| QQT | | QQT | | LYNNT | | NQQT | |
| GVAGSLVSGQN | 301 | GSGIAAQRTQN | 486 | GSGAVHLTA | 671 | GSGQNQAN | 856 |
| QQT | | QQT | | QNQQT | | WVKVQT | |
| GRGGIPQSGQN | 302 | GSTPAGVGGQN | 487 | GSLVSTQSG | 672 | GSGHLFQSG | 857 |
| QQT | | QQT | | QNQQT | | QNQQT | |
| GSGQYASSIPNQ | 303 | GSGQNQQTSTG | 488 | GSGVSARMV | 673 | GSGQNRGISI | 858 |
| QT | | VHS | | QNQQT | | SQQT | |
| GTDFGRQSSQN | 304 | GSGQIRQLVDN | 489 | GSGQTRMPL | 674 | GSGTHYDNR | 859 |
| QQT | | QQT | | ANQQT | | QNQQT | |
| GIFMQTPSGQNQ | 305 | GSLIGMQSGQN | 490 | GSGISSRNMQ | 675 | GSGQNQQTS | 860 |
| QT | | QQT | | NQQT | | TTPLP | |
| GSGQNQQTRLV | 306 | GSGQIKGKMDN | 491 | GSGEKVHSG | 676 | GSGQVHASQ | 861 |
| DLT | | QQT | | QNQQT | | VNQKT | |
| GTREMPLSGQN | 307 | GSGSDMSSWQN | 492 | GSGQNQQKL | 677 | GSSGHRESG | 862 |
| QQT | | QQT | | SSMST | | QNQQT | |
| GSRLVHVHGQN | 308 | GRGQNQQHTGL | 493 | GSGQNQQTG | 678 | GLSAEKSSG | 863 |
| QQT | | ATT | | QHMRV | | QNQQT | |
| GSGRLVPNGPN | 309 | GSGQNQQTLYS | 494 | GSGMIHTTA | 679 | GSGQEHRSL | 864 |
| QQT | | SNT | | QNQQT | | ANQQT | |
| GSGYLRESPQN | 310 | GSGQTQVLKSN | 495 | GSGQNWPAL | 680 | GSGQTVVRI | 865 |
| QQT | | QQT | | KGQQT | | ANQQT | |
| GARIQNASGQK | 311 | GSRTLSNVGQN | 496 | GASHMSISG | 681 | GSGQNVSSV | 866 |
| QQT | | QQT | | QNQQT | | HRQQT | |
| GLSNPMPSGQN | 312 | GSGVQHSLPQN | 497 | GSDQNQQLG | 682 | GSGASRMSI | 867 |
| QQT | | QQT | | YSKQT | | QNQQT | |
| GSTVQDTRGQN | 313 | GNYLHQASGQN | 498 | GIPSIRESGQ | 683 | GVAFIGSSGQ | 868 |
| QQT | | QQT | | NQQT | | NQQT | |
| GPFGMPSSGQN | 314 | GSGGTSVHQQN | 499 | GSGIPSVKFQ | 684 | GSGQNQQTV | 869 |
| QQT | | QQT | | NQQT | | PTRQT | |
| GSGQNHGVLSN | 315 | GMDHSRPSGQN | 500 | GSGQNQQTS | 685 | GSGQAAKSS | 870 |
| QQT | | QQT | | VSQNV | | QNQQT | |
| GSGYSMSQAQN | 316 | GSGQNQQSMGT | 501 | GSGQNQQIG | 686 | GSGQNQQVA | 871 |
| QQT | | FTT | | ESRMT | | IRTST | |
| GSGMLTHTLQN | 317 | GSGQNQQTPLR | 502 | GSGSSSMSFQ | 687 | GSVHMQNA | 872 |
| QQT | | PPT | | NQQT | | GQNQQT | |
| GRGSPHASRQN | 318 | GSGQNQHHSVS | 503 | GSGQKQERA | 688 | GSGMRQAG | 873 |
| QQT | | QQT | | VSKQT | | VQNQQT | |
| GLSWPSTSGQN | 319 | GSGQLRSLSTNQ | 504 | GCTTRLNSG | 689 | GSGQNQQVG | 874 |
| QQT | | QT | | QNQQT | | GKTVT | |
| GNSMERTSGQN | 320 | GSGSPRQLSQN | 505 | GSGQNQQIIS | 690 | GVHDMRVS | 875 |
| QQT | | QQT | | TKIT | | GQNQQT | |
| GSGMSPSTLQN | 321 | GSGQNQQTTAS | 506 | GSGQNQQKS | 691 | GSGQHVSVA | 876 |
| QQT | | SHT | | LNGNT | | NNQQT | |
| GSGHGQVLSQN | 322 | GRGQVVSTHQN | 507 | GSGIPAPRLQ | 692 | GSAAMSVRG | 877 |
| QQT | | QQT | | NQQT | | QNQQT | |
| GRGQIYSTGGN | 323 | GSAQVSMVGQN | 508 | GSGQIRESM | 693 | GVSRGGPSG | 878 |
| QQT | | QQT | | GNQQT | | QNQQT | |
| GVVAAHNSGQN | 324 | GSSTLVTIGKNQ | 509 | GSGQNSGVH | 694 | GSGQMVHTI | 879 |
| QQT | | QT | | FNQQT | | GNQQT | |
| GDSSLRHSGQN | 325 | GFAHQASSGQN | 510 | GSGQNIGHSL | 695 | GRGGSMAET | 880 |
| QQT | | QQT | | PQQT | | QNQQT | |
| GSLVSQGAGQN | 326 | GSGQPVLSISNQ | 511 | GSGERSISVQ | 696 | GSGHTNPTR | 881 |
| QQT | | QT | | NQQT | | QNQQT | |
| GSLLQAHSGQN | 327 | GSGQSHRSELN | 512 | GSGLKPNVL | 697 | GSGEAARYE | 882 |
| QQT | | QQT | | QNQQT | | QNQQT | |
| GSGHIYVGIQNQ | 328 | GSSVGSPIGQNQ | 513 | GSGQVAYAQ | 698 | GSGQNERHL | 883 |
| QT | | QT | | GNQQT | | VLQQT | |
| GHHTTVQSGQN | 329 | GSGMPIRNVQN | 514 | GSGQSSYGS | 699 | GSGQNQQSK | 884 |
| QQT | | QQT | | GNQQT | | QQVLT | |
| GSRQSKRNELN | 330 | GSSTRVDSGQN | 515 | GSGQNQAMT | 700 | GSGQARAHR | 885 |
| QQT | | QQT | | HGDQT | | GNQQT | |
| GSGQNQQHVSS | 331 | GSGQNQQTAMR | 516 | GSGQNQALV | 701 | GSGQNQQPL | 886 |
| PRT | | STT | | SMGQT | | DTSRT | |
| GSSKELLWGQN | 332 | GSGQNQQHSSS | 517 | GSGQNPSFM | 702 | GSGQNQQLA | 887 |
| QQT | | HLT | | RGQQT | | NMVTT | |
| GSLSTPSSGQNQ | 333 | GSRNGHAVGQN | 518 | GSGQNQQSH | 703 | GSGQMKDL | 888 |
| QT | | QQT | | LRTNT | | HRNQQT | |
| GSIGYAGQGQN | 334 | GLGAYQSSGQN | 519 | GYTRLETSG | 704 | GSGQNQHLS | 889 |
| QQT | | QQT | | QNQQT | | SFVQT | |
| GSGQNQRVSNS | 335 | GPGLSGHSGQN | 520 | GSGQSYDMR | 705 | GSGQNQQPS | 890 |
| QQT | | QQT | | GNQQT | | SRVTT | |
| GSGYASHVQQN | 336 | GSTGIVSSGQNQ | 521 | GSRTTQDIGQ | 706 | GSGQNQQLA | 891 |
| QQT | | QT | | NQQT | | ITLGT | |
| GSGEYSRSGQN | 337 | GSRTTQVIGQNQ | 522 | GSGHPYKAA | 707 | GSGQNQQTV | 892 |
| QQT | | QT | | QNQQT | | GNPAT | |
| GSVSTHSSGQN | 338 | GSGLLHRAQQN | 523 | GRLSNAHGG | 708 | GSGQNQGRA | 893 |
| QQT | | QQT | | QNQQT | | HPMQT | |
| GSGQNQHSLGN | 339 | GSGQNAQQAAA | 524 | GSGQNQRAV | 709 | GSGQLIASV | 894 |
| YQT | | QQT | | LNDQT | | VNQQT | |
| GSGGLDTRGQN | 340 | GSGQNQQSALR | 525 | GGSHTYGGG | 710 | GSSVRSLVG | 895 |
| QQT | | TQT | | QNQQT | | QNQQT | |
| GNILHATSGQN | 341 | GSGFLSDTRQN | 526 | GSSVNSMIG | 711 | GGAGSAHSG | 896 |
| QQT | | QQT | | QNQQT | | QNQQT | |
| GSGQSYTMTQN | 342 | GSGLLYHDQQN | 527 | GNSSMMGSG | 712 | GSDQNQQT | 897 |
| QQT | | QQT | | QNQQT | | MSSTRT | |
| GSGQNQHSAPN | 343 | GSGQNQHYSLH | 528 | GNRDRPSSG | 713 | GSGQNQQM | 898 |
| SQT | | KQT | | QNQQT | | AGAFRT | |
| GSGQNQQTMDH | 344 | GSGHSPLPQQN | 529 | GSGNMHASR | 714 | GSLGNLQRG | 899 |
| NRT | | QQT | | QNQQT | | QNQQT | |
| GSNGGVGTGQN | 345 | GNGHSMRPNQN | 530 | GFIFPKVSGQ | 715 | GSGPSISHGQ | 900 |
| QQT | | QQT | | NQQT | | NQQT | |
| GAGSIIPSGQNQ | 346 | GSGLKWSTLQN | 531 | GSGQNQQLK | 716 | GSGQNQQSS | 901 |
| QT | | QQT | | NSTST | | FNVQT | |
| GSGQTHGGQHN | 347 | GSGQMGRQAV | 532 | GSGQNQQSQ | 717 | GSGQNQQTG | 902 |
| QQT | | NQQT | | YMPRT | | QATHN | |
| GSNLSFQSGQN | 348 | GSGQNQQTSGV | 533 | GSGQRMADI | 718 | VSGSPHSKA | 903 |
| QQT | | LTL | | GNQQT | | QNQQT | |
| GATLQVHSGQN | 349 | GSGQNQQALHN | 534 | GSGQNQSHY | 719 | CSGSPHSKA | 904 |
| QQT | | PHT | | PSQQT | | QNQQT | |
| GSGFNQRSEQN | 350 | GSGQNQQVIPNS | 535 | GSDGKMHRG | 720 | GSGSPHRKA | 905 |
| QQT | | KT | | QNQQT | | QNQQT | |
| GSGSLRDFDQN | 351 | GSPLQDRVGQN | 536 | GSGSVGFIGQ | 721 | GRGSPHSKA | 906 |
| QQT | | QQT | | NQQT | | QNQQT | |
| GSGDSITGKQNQ | 352 | GSGQNQYSSTN | 537 | GLHGMTLSG | 722 | GSGSPHSKA | 907 |
| QT | | PQT | | QNQQT | | QNKQT | |
| GSGQDRNIVQN | 353 | GAMTVTISGQN | 538 | GSDQSKRGD | 723 | GSGSPHSKA | 908 |
| QQT | | QQT | | SNQQT | | QTQQT | |
| GSGLSHSHQQN | 354 | GSGQNQQLQTLI | 539 | GSLFLATGG | 724 | GSGSTHASR | 909 |
| QQT | | RT | | QNQQT | | QNQQT | |
| GSGQNQQTGMS | 355 | GSGLRQTSQQN | 540 | GSGQNQQPS | 725 | GSGSPHKYG | 910 |
| SVK | | QQT | | AFSKT | | QNQQT | |
| GSVTHGISGQNQ | 356 | GSGQNQQTGLR | 541 | GSGQLPQSG | 726 | GSGSPHKFG | 911 |
| QT | | QQT | | LNQQT | | QNQQT | |
| GVVAHQPSGQN | 357 | GSGQTRQMKDN | 542 | GSGSKQNAL | 727 | VSGSPHKFG | 912 |
| QQT | | QQT | | QNQQT | | QNQQT | |
| GSGPILGQLQNQ | 358 | GSGQNHGLQSG | 543 | GSGQRRELS | 728 | GSGSPHSKA | 913 |
| QT | | QQT | | QNQQT | | QNHQT | |
| GSGHVPNSGLN | 359 | GSGQSHRQPEN | 544 | GSGQREPKA | 729 | GSGSPHSKA | 914 |
| QQT | | QQT | | SNQQT | | QHQQT | |
| GDAGVRSSGQN | 360 | GSGQDRHIVQN | 545 | GSGQNQQHP | 730 | GSGSPHKTY | 915 |
| QQT | | QQT | | STQQT | | QNQQT | |
| GSGSQLMSLQN | 361 | GSGQNQQLPHS | 546 | GSQSTLGLG | 731 | VSGSPHASR | 916 |
| QQT | | NLT | | QNQQT | | QNQQT | |
| GSGLDYSQRQN | 362 | GSGQLSVPYDN | 547 | GSGQNQQMP | 732 | GSGSPHKFG | 917 |
| QQT | | QQT | | GLSST | | KNQQT | |
| GSGQSSGRLINK | 363 | GSGRNPQTQPL | 548 | GSGQNQQTV | 733 | GSGSPHASR | 918 |
| QT | | QQT | | GGKNL | | QNQHT | |
| GSSVSPSSGQNQ | 364 | GSGQPYSTGLN | 549 | GSSREFHSGQ | 734 | GSHSPHKSG | 919 |
| QT | | QQT | | NQQT | | QNQQT | |
| GSGQVVGLSGN | 365 | GSGQNQQTHGG | 550 | GSGQNQQTV | 735 | GSGQNQQRR | 920 |
| QQT | | LRD | | PSNLV | | MSPST | |
| GSNMGVPLGQN | 366 | GAYGMVSSGQN | 551 | GSGQNAYSS | 736 | GSGSPHSKP | 921 |
| QQT | | QQT | | QAQQT | | QNQQT | |
| GSFYPSSTGQNQ | 367 | GSGIQSSYSQNQ | 552 | GSGQNKDHS | 737 | GSGSPHKFG | 922 |
| QT | | QT | | TRRQT | | QKQQT | |
| GSGQNQQTRLT | 368 | GPRLSDQSGQN | 553 | GQLGSVGSG | 738 | VSGSPHGAR | 923 |
| DLT | | QQT | | QDQQT | | QNQQT | |
| GPTNGRSSGQN | 369 | GSGQNQQTHPS | 554 | GSGQHAAPG | 739 | GSGSPHSKA | 924 |
| QQT | | PCT | | HNQQT | | QKQQT | |
| GSGLLHGKLQN | 370 | GSGQSFQMHTN | 555 | GSGQNQQTS | 740 | GSHSPHKRG | 925 |
| QQT | | QQT | | QSPPT | | QNQQT | |
| GANMGHVSGQ | 371 | GSGQNQQTGNP | 556 | GSGNYRDHE | 741 | GSGQNRQRL | 926 |
| NQQT | | KHT | | QNQQT | | KGLET | |
| GSGQNQQSGRG | 372 | GFSSAVHSGQN | 557 | GSGQHSNQH | 742 | GSGSPHKLG | 927 |
| DLT | | QQT | | VNQQT | | QNQQT | |
| GSHGHYASGQK | 373 | GSGQNQQTSMS | 558 | GSGQTARNG | 743 | GSGSPHKTS | 928 |
| QQT | | NAT | | INQQT | | KNQQT | |
| GSGDLRISPQNQ | 374 | GSGQDMKQHH | 559 | GSGQNQQHY | 744 | GSGSPHKIGQ | 929 |
| QT | | NQQT | | GSQGT | | NQQT | |
| GSGMPVILGQN | 375 | GLRLSTPSGQNQ | 560 | GSGSPQASR | 745 | GSGQDSPHV | 930 |
| QQT | | QT | | QNQQT | | RNQQT | |
| GRGVITSSGQNH | 376 | GSGQNQQTSVY | 561 | GSGFSHSMG | 746 | GSGSPHKTS | 931 |
| QT | | MNT | | KNQQT | | QNQQT | |
| GSGHSVSGPQN | 377 | GSGQNQYSQSS | 562 | GSGQSHSLET | 747 | GSGSPHASR | 932 |
| QQT | | MQT | | NQQT | | KNQQT | |
| GSRNGHTVGRN | 378 | GSGQNQQSMAD | 563 | GTEQTRQSG | 748 | GSHSPHKSG | 933 |
| QQT | | HTT | | QNQQT | | QKQQT | |
| GAGVHMVSGQ | 379 | GWERSFVSGQN | 564 | GSGRHLASV | 749 | GSGSPHKTS | 934 |
| NQQT | | QQT | | QNQQT | | QKQQT | |
| GSGQNHRPSVL | 380 | GLLAGKSSGQN | 565 | GLGSKNHSG | 750 | GSGSPHVRG | 935 |
| QQT | | QQT | | QNQQT | | QNKQT | |
| GSGSPRDSIQNQ | 381 | GKSFVPQSGQN | 566 | GSGQNQQTS | 751 | GSGSPHKTT | 936 |
| QT | | QQT | | HFPSA | | QNQQT | |
| GSGQGIHSSVNQ | 382 | GSGQMQSAGSN | 567 | GSGQLSGTP | 752 | GSGPVRALR | 937 |
| QT | | QQT | | QNQQT | | QNQQT | |
| GSGQQLSITPNQ | 383 | GSDQNQRLTSS | 568 | GSGQNQQAP | 753 | GSGSPHVRG | 938 |
| QT | | MQT | | HKKET | | QKQQT | |
| GGYHSQTSGQN | 384 | GESRAVLSGQN | 569 | GSGQNQQTL | 754 | CSGSPHKTS | 939 |
| QQT | | QQT | | RGSLE | | QNQQT | |
| CSHSPHKSGQN | 940 | DAGSPHSKAQN | 1909 | GSGSPHASR | 2019 | GNDSPHKSV | 2129 |
| QQT | | QQ | | QNQQ | | QNQQ | |
| GHDSPHKSGQN | 1800 | GSGSPHSKGQN | 1910 | GSGSPHASR | 2020 | GHDSPHKSA | 2130 |
| QQ | | QQ | | QNKQ | | QNYQ | |
| GSGSPHSKAQN | 1801 | DGGSPHSKAQN | 1911 | GSGSPHVKIQ | 2021 | GSASPHSKA | 2131 |
| QQ | | QQ | | NQQ | | LNQQ | |
| GSGSPHSKAQN | 1802 | ASGSPHSKAHN | 1912 | GSGSPHSKA | 2022 | GHESPHKSA | 2132 |
| RH | | QQ | | KNQQ | | QNRQ | |
| GSGSPHSKVQN | 1803 | GSGSPHSKAQN | 1913 | GSGSPHKKN | 2023 | GQDSPHKIG | 2133 |
| QQ | | TY | | QNQQ | | QNQQ | |
| MSGSPHSKAQN | 1804 | GSGSPHSKSQN | 1914 | GSGSPHVRM | 2024 | GHDSPHKSG | 2134 |
| QQ | | QH | | QNQQ | | QNHL | |
| GRGSPHSKAQN | 1805 | GGGSPHSKAQD | 1915 | GSGSPHASR | 2025 | GHDSPHKSG | 2135 |
| QQ | | KQ | | QKQQ | | QYQH | |
| RNGSPHSKAQN | 1806 | GSGSPHSKAQN | 1916 | GHSSPHRSG | 2026 | GNDSPHKSV | 2136 |
| QQ | | HL | | QNQQ | | QNHQ | |
| GSGSPHSKARD | 1807 | GSGSPHSKAQIG | 1917 | GSGSPHTRG | 2027 | GHDSPHKSG | 2137 |
| QQ | | M | | QNQQ | | QNQW | |
| GSGSPHSKAPNL | 1808 | GSGSPHSKALN | 1918 | CSGSPHSKA | 2028 | GHDSPHKSV | 2138 |
| Q | | KQ | | QNQQ | | QNQH | |
| TSGSPHSKAQN | 1809 | GGGSPHSKAQN | 1919 | GSGSPHRKA | 2029 | GHDSPHKSG | 2139 |
| QQ | | PQ | | QNQQ | | QNQH | |
| GSGSPHSKAHV | 1810 | GTGSPHSKAPN | 1920 | GSGSPHSKA | 2030 | GHDSPHKSG | 2140 |
| RQ | | QL | | QNKQ | | QTRQ | |
| GSGSPHSKAPNQ | 1811 | GSGSPHSKAQL | 1921 | GSGSPHSKA | 2031 | GHDSPHKSG | 2141 |
| H | | QQ | | QTQQ | | QNLH | |
| ISGSPHSKAQNQ | 1812 | GGGSPHSKAQY | 1922 | CSGSPHKTSQ | 2032 | GHDSPHKSA | 2142 |
| Q | | QQ | | NQQ | | QNQE | |
| GPGSPHSKAHN | 1813 | GGGSPHSKAQH | 1923 | CSHSPHKSG | 2033 | GHDSPHKSG | 2143 |
| QQ | | QQ | | QNQQ | | QHLQ | |
| GSGSPHSKTQSQ | 1814 | GSGSPHSKAQR | 1924 | GQSSPHRSG | 2034 | GHDSPHKSR | 2144 |
| Q | | MS | | QNQQ | | LNQP | |
| ESGSPHSKAQN | 1815 | GSGSPHSKAQGI | 1925 | GRGSPHASR | 2035 | GQDSPHKSG | 2145 |
| QQ | | L | | QNQQ | | QNQD | |
| GSGSPHSKAQPA | 1816 | GSGSPHSKAQD | 1926 | GSGSPHASR | 2036 | GHDSPHKSG | 2146 |
| K | | RQ | | KNQQ | | RNQQ | |
| SSGSPHSKAQNQ | 1817 | GSGSPHSKARD | 1927 | GSGSPHASR | 2037 | GHDSPHKSG | 2147 |
| Q | | WQ | | QNQH | | QNLL | |
| GNGSPHSKAQN | 1818 | GSGSPHSKAQN | 1928 | GSGSPHKIG | 2038 | GHDSPHKSG | 2148 |
| QQ | | TH | | KNQQ | | QLVI | |
| GSGSPHSKSQTQ | 1819 | GSGSPHSKAQE | 1929 | GSGSPHKFG | 2039 | GHDSPHKSR | 2149 |
| Q | | RS | | QKQQ | | QSQQ | |
| ASGSPHSKAQN | 1820 | GSGSPHSKAQN | 1930 | GSGSPHKFG | 2040 | GHDSPHKSG | 2150 |
| QQ | | YQ | | QNQQ | | RTQE | |
| GSGSPHSKAQN | 1821 | GSGSPHSKAQR | 1931 | GSGSPHKIGQ | 2041 | GHDSPHKSV | 2151 |
| LA | | TC | | NQQ | | QTHQ | |
| GSGSPHSKSQNQ | 1822 | GSGSPHSKAQIG | 1932 | GSGSPHKLG | 2042 | GHDSPHKSG | 2152 |
| L | | H | | QNQQ | | QNQP | |
| NSGSPHSKAQN | 1823 | GSGSPHSKAQG | 1933 | GSGSPHKTSK | 2043 | GHDSPHKSG | 2153 |
| QQ | | AI | | NQQ | | QTQQ | |
| GSGSPHSKAQG | 1824 | GSGSPHSKAQV | 1934 | GSGSPHKTSQ | 2044 | GPDSPHKIGQ | 2154 |
| QQ | | PP | | KQQ | | NQQ | |
| VSGSPHSKAQN | 1825 | GSGSPHSKAQV | 1935 | GSGSPHKTSQ | 2045 | GHDSPHKSV | 2155 |
| QQ | | QQ | | NQQ | | QNQQ | |
| GSGSPHSKALNR | 1826 | GSGSPHSKALM | 1936 | GSGSPHKTT | 2046 | GHDSPHKSR | 2156 |
| Q | | RQ | | QNQQ | | QDQH | |
| LSGSPHSKAQN | 1827 | GSGSPHSKAQY | 1937 | GSGSPHKTY | 2047 | GPDSPHKSG | 2157 |
| QQ | | SV | | QNQQ | | QKQQ | |
| GSGSPHSKAHN | 1828 | GSGSPHSKVPNL | 1938 | GSGSPHKYG | 2048 | GHDSPHKSR | 2158 |
| QQ | | Q | | QNQQ | | QSQH | |
| GSGSPHSKTQN | 1829 | GSGSPHSKAQAI | 1939 | GSGSPHSKA | 2049 | GHDSPHKSV | 2159 |
| QQ | | T | | QHQQ | | QNQL | |
| GGGSPHSKAQT | 1830 | GSGSPHSKAQK | 1940 | GSGSPHSKD | 2050 | GYDSPHKSG | 2160 |
| QQ | | TL | | QNQQ | | QYQH | |
| GSGSPHSKAQNP | 1831 | GSGSPHSKAQN | 1941 | GSGSPHSKPQ | 2051 | GHDSPHKSR | 2161 |
| P | | QW | | NQQ | | QNQQ | |
| GSGSPHSKAQN | 1832 | GSGSPHSKAQL | 1942 | GSGSPHVRG | 2052 | GHDSPHKSW | 2162 |
| LQ | | HH | | QKQQ | | VRQQ | |
| GGGSPHSKAQN | 1833 | GSGSPHSKAQNI | 1943 | GSGSPHVRG | 2053 | GHESPHKSG | 2163 |
| QQ | | I | | QNKQ | | QNQH | |
| GSGSPHSKAQY | 1834 | MEGSPHSKAQN | 1944 | GSHSPHKRG | 2054 | GHDSPHKIG | 2164 |
| QQ | | QQ | | QNQQ | | HNQQ | |
| GGGSPHSKAQN | 1835 | GSGSPHSKAQG | 1945 | GSHSPHKSG | 2055 | GHDSPHKSN | 2165 |
| KQ | | HH | | QKQQ | | AWQQ | |
| GSGSPHSKAQD | 1836 | GSGSPHSKAQS | 1946 | GSHSPHKSG | 2056 | GHDSPHKSG | 2166 |
| QE | | KV | | QNQQ | | QSVP | |
| KSGSPHSKAQN | 1837 | GSGSPHSKAQLP | 1947 | VSGSPHASR | 2057 | GHESPHKSG | 2167 |
| QQ | | S | | QNQQ | | QNIQ | |
| GGGSPHSKAQN | 1838 | GSGSPHSKAIGK | 1948 | VSGSPHGAR | 2058 | GHDSPHKSV | 2168 |
| QL | | Q | | QNQQ | | QNHL | |
| GSGSPHSKAQN | 1839 | GGGSPHSKSQN | 1949 | VSGSPHKFG | 2059 | GHDSPHKIG | 2169 |
| HQ | | QQ | | QNQQ | | LDQQ | |
| GSGSPHSKAQD | 1840 | GSGSPHSKAQAI | 1950 | GSGSPHSKA | 2060 | ASGSPHSKA | 2170 |
| QQ | | H | | QYYV | | QHQQ | |
| GGGSPHSKSQN | 1841 | GSGSPHSKAQH | 1951 | GSGSPHSKLR | 2061 | GHDSPHKRG | 2171 |
| QL | | GL | | RQQ | | PDQQ | |
| GNGSPHSKAQN | 1842 | GSGSPHSKAQF | 1952 | GSGSPHSKA | 2062 | GMGSPHSKT | 2172 |
| KQ | | MC | | GCGQ | | QNQQ | |
| GSGSPHSKGHW | 1843 | VSGSPHSKAQG | 1953 | GSGSPHSRA | 2063 | GHDSPHKSG | 2173 |
| QQ | | QQ | | QNQQ | | ESQQ | |
| GSGSPHSKAPNQ | 1844 | GGGSPHSKAQN | 1954 | GSGSPHSKRL | 2064 | GHDSPHKHG | 2174 |
| Q | | QM | | RQQ | | QNHQ | |
| GSGSPHSKAQN | 1845 | GSGSPHSKAQH | 1955 | GSGSPHSLRR | 2065 | GTGSPHSKA | 2175 |
| QL | | LQ | | NQQ | | QNQL | |
| GSGSPHSKRPEQ | 1846 | ENGSPHSKAQN | 1956 | GSGSPHSRGR | 2066 | GHDSPHKSV | 2176 |
| Q | | QQ | | NQQ | | QNKQ | |
| GSGSPHSKAQRT | 1847 | GSGSPHSKTQN | 1957 | GSGSPHSSRR | 2067 | GQVSPHKSG | 2177 |
| M | | HQ | | NQQ | | QNQQ | |
| GNGSPHSKAQN | 1848 | GSGSPHSKAQP | 1958 | GSGSPHSKAF | 2068 | GHDSPHKSG | 2178 |
| QH | | AR | | RLQ | | QRQL | |
| HSGSPHSKAQN | 1849 | GSGSPHSKAQSL | 1959 | GSCSPHRKA | 2069 | GHDSPHKIG | 2179 |
| QQ | | Q | | QNQQ | | QNQL | |
| GGGSPHSKALN | 1850 | GSGSPHSKSQSQ | 1960 | GSGSPHFLRQ | 2070 | GHDSPHKSG | 2180 |
| QQ | | L | | NQQ | | QIIV | |
| GSGSPHSKALH | 1851 | GSASPHSKAHS | 1961 | GSGSPHSLRF | 2071 | GYDSPHKSG | 2181 |
| QH | | QQ | | NQQ | | QKQS | |
| GTGSPHSKAQN | 1852 | GSGSPHSKAQM | 1962 | GSGSPHSKW | 2072 | GNGSPHSKA | 2182 |
| HQ | | PS | | LLQQ | | QNQE | |
| GSGSPHSKAQH | 1853 | GSGSPHSKAQG | 1963 | GSGSPHSKRR | 2073 | GDDSPHKSV | 2183 |
| RI | | SL | | LQQ | | QNQQ | |
| GSGSPHSKAQYI | 1854 | GSGSPHSKSQN | 1964 | GSGSPHSKA | 2074 | GHDSPHKSV | 2184 |
| H | | QQ | | QRKL | | QSHQ | |
| GGGSPHSKAHN | 1855 | GNGSPHSKSQN | 1965 | GSGSPHSKAL | 2075 | GHDSPHKSG | 2185 |
| QQ | | QQ | | RRQ | | QFVV | |
| GSGSPHSKAQKF | 1856 | GSGSPHSKAQV | 1966 | GSGSPHSKA | 2076 | GHDSPHKSR | 2186 |
| E | | PA | | QRLR | | QNLQ | |
| ESGSPHSKAQN | 1857 | GNGSPHSKAQN | 1967 | GSGSPHSKA | 2077 | GHNSPHKSG | 2187 |
| HQ | | LQ | | QRRL | | QNQE | |
| GSGSPHSKAQFP | 1858 | GSGSPHSKAQD | 1968 | GSGSPHSKA | 2078 | GHDSPHKSG | 2188 |
| S | | KQ | | RRQQ | | QSQP | |
| PSGSPHSKAQNQ | 1859 | GSGSPHSKAHY | 1969 | GSGSPHSKA | 2079 | GHESPHKSG | 2189 |
| Q | | QQ | | RRLQ | | QNEQ | |
| GNGSPHSKAQN | 1860 | GSGSPHSKAQV | 1970 | GSGSPHSKSR | 2080 | GHDSPHKSG | 2190 |
| PL | | PS | | RQQ | | QNQL | |
| GGGSPHSKAQS | 1861 | GGGSPHSKAQN | 1971 | GLLSPHWKA | 2081 | GHDSPHKSA | 2191 |
| QQ | | HQ | | QNQQ | | QNLL | |
| GSGSPHSKAQAI | 1862 | GSGSPHSKARG | 1972 | GSGSPHSKA | 2082 | ASGSPHSKAI | 2192 |
| K | | EQ | | RLRQ | | NQQ | |
| GSGSPHSKGQN | 1863 | GGGSPHSKAQY | 1973 | GSGSPHSKAS | 2083 | GNGSPHKRG | 2193 |
| RQ | | QH | | KRQ | | QNQQ | |
| GSGSPHSKAQSQ | 1864 | GSGSPHSKAPG | 1974 | GSGSPHVRR | 2084 | GHDSPHKSG | 2194 |
| Q | | QQ | | QNQQ | | QSLQ | |
| GSVSPHGKAQN | 1865 | KNGSPHSKAQN | 1975 | GSGSPHSKA | 2085 | GHDSPHKSA | 2195 |
| QL | | QQ | | QLYR | | QNHQ | |
| ASGSPHSKAQN | 1866 | GSGSPHSKRLEQ | 1976 | GSGSPHSKA | 2086 | GHDSPHKSG | 2196 |
| QL | | Q | | QLTV | | RNRQ | |
| RSGSPHSKAQN | 1867 | GSGSPHSKAQN | 1977 | GHDSPHKRG | 2087 | GHDSPHKYG | 2197 |
| QQ | | QS | | QHRQ | | QNEQ | |
| GSGSPHSKAQY | 1868 | GSGSPHSKAQK | 1978 | GHDSPHKSG | 2088 | GNGSPHSKA | 2198 |
| QH | | VI | | QKQQ | | PNLQ | |
| GSGSPHTKAQN | 1869 | GSGSPHSKAQN | 1979 | GHDSPHKSG | 2089 | GHDSPHKSQ | 2199 |
| PQ | | ND | | LTQQ | | QNQQ | |
| GSGSPHSKGQNP | 1870 | GSGSPHSKAQS | 1980 | GDDSPHKSG | 2090 | GHDSPHKSV | 2200 |
| P | | VH | | RNQQ | | QSKQ | |
| GSGSPHSKAQH | 1871 | GSGSPHSKAQPL | 1981 | GHDSPHKSG | 2091 | GNDSPHKIG | 2201 |
| QL | | G | | LNQQ | | HNQQ | |
| GSGSPHSKAQSP | 1872 | KEGSPHSKAQN | 1982 | GHESPHKSA | 2092 | GGGSPHSKA | 2202 |
| P | | QQ | | QNHQ | | QDQQ | |
| GSGSPHSKAQA | 1873 | GSGSPHSKAHN | 1983 | GHDSPHKSA | 2093 | GQDSPHKSG | 2203 |
| KL | | QE | | QNQW | | QNPL | |
| GSGSPHSKTKSQ | 1874 | GSGSPHSKAQIQ | 1984 | GHDSPHKSG | 2094 | ASGSPHSKA | 2204 |
| Q | | Q | | QNTH | | QNHQ | |
| GSGSPHSKAQD | 1875 | GSGSPHSKAQV | 1985 | GHDSPHKSG | 2095 | GHDSPHKSG | 2205 |
| RP | | RN | | RRRQ | | RDQK | |
| GIGSPHSKAQNL | 1876 | GSGSPHSKAPSN | 1986 | GHDSPHKSA | 2096 | GHDSPHKSV | 2206 |
| G | | Q | | QNQQ | | HNQQ | |
| GSGSPHSKAQAF | 1877 | GSGSPHSKAQV | 1987 | GHDSPHKSG | 2097 | GHDSPHKSG | 2207 |
| H | | GH | | QYQQ | | QWKR | |
| GSGSPHSKAQK | 1878 | GSGSPHSKAQR | 1988 | GNYSPHKIG | 2098 | GSGSPHSKA | 2208 |
| QQ | | DI | | QNQQ | | ENRQ | |
| GSGSPHSKAQN | 1879 | GSGSPHSKAQM | 1989 | GHDSPHKSR | 2099 | GHDSPHKSG | 2209 |
| AQ | | PN | | QNDQ | | QSQQ | |
| WSGSPHSKAQN | 1880 | AIGSPHSKAQNQ | 1990 | GHDSPHKSG | 2100 | GHDSPHKSR | 2210 |
| QQ | | Q | | QIRQ | | QAQQ | |
| GSGSPHSKAHN | 1881 | GSGSPHSKARG | 1991 | GHDSPHKIG | 2101 | GHDSPHKSV | 2211 |
| QL | | LQ | | QNQH | | QNHQ | |
| GNGSPHSKAQN | 1882 | GSGSPHSKLQK | 1992 | GYDSPHKSG | 2102 | GHDSPHKSK | 2212 |
| HQ | | QQ | | QKQQ | | QNQQ | |
| GGGSPHSKAQN | 1883 | GSGSPHSKAPSL | 1993 | GHDSPHKSG | 2103 | GHDSPHKSA | 2213 |
| LQ | | Q | | QSVQ | | QNQL | |
| GSGSPHSKAQK | 1884 | GSGSPHSKAQR | 1994 | GHESPHKSG | 2104 | GHDSPHKSG | 2214 |
| LN | | DQ | | RSQQ | | QTQP | |
| GGGSPHSKSQN | 1885 | GSGSPHSKNRD | 1995 | GHDSPHKSG | 2105 | GHDSPHKLW | 2215 |
| QH | | QQ | | QNKL | | INQQ | |
| GSGSPHSKSQNV | 1886 | GSGSPHSKAQA | 1996 | GHDSPHKTG | 2100 | GPDSPHKSG | 2216 |
| Q | | KG | | QNQQ | | QNQQ | |
| GSGSPHSKAQA | 1887 | GSGSPHSKAQS | 1997 | GRGSPHKRG | 2107 | GHDSPHKSV | 2217 |
| QQ | | AH | | QNQQ | | QKQL | |
| DSGSPHSKAQN | 1888 | GNGSPHSKSQN | 1998 | GSGSPHTKA | 2108 | GHPSPHWKG | 2218 |
| QQ | | QH | | QNPP | | QNQQ | |
| ASGSPHSKAPNQ | 1889 | GSGSPHSKSQN | 1999 | GQDSPHKSG | 2109 | GHDSPHKSG | 2219 |
| Q | | HQ | | QHQQ | | RNQL | |
| GSGSPHSKAQTP | 1890 | RSGSPHSKAQD | 2000 | GHDSPHKSG | 2110 | GSGSPHSKV | 2220 |
| P | | QQ | | QIQH | | QDQQ | |
| IDGSPHSKAQNQ | 1891 | GSGSPHSKAQST | 2001 | GHDSPHKSG | 2111 | GHDSPHKMG | 2221 |
| Q | | M | | PRQQ | | RNQQ | |
| GSGSPHNKAQN | 1892 | GSGSPHSKAQR | 2002 | GHDSPHKSG | 2112 | GHDSPHKSGI | 2222 |
| HQ | | EM | | HTQQ | | SIQ | |
| GSGSPHSKAQPP | 1893 | GGGSPHSKSQN | 2003 | GHDSPHKSG | 2113 | GHDSPHKSV | 2223 |
| A | | RQ | | QRQH | | QNLQ | |
| GSGSPHSKAQER | 1894 | GSGSPHSKAQY | 2004 | GSGSPHTKA | 2114 | GHDSPHKMA | 2224 |
| P | | RA | | QNQQ | | HNQQ | |
| GSGSPHSKAQD | 1895 | GGGSPHSKAQR | 2005 | GHDSPHKSA | 2115 | GHDSPHKHG | 2225 |
| LQ | | QQ | | QSQQ | | QNQQ | |
| GGGSPHSKAQN | 1896 | GSGSPHSKNQW | 2006 | GHESPHKSG | 2116 | GHDSPHKSV | 2226 |
| PP | | QQ | | QNQQ | | QSQQ | |
| GSGSPHSKAQA | 1897 | GSGSPHSKAQR | 2007 | GHDSPHKSL | 2117 | GHDSPHKSG | 2227 |
| MH | | MN | | QNQQ | | QTVC | |
| GSGSPHSKALN | 1898 | GSGSPHAKAQN | 2008 | GHGSPHSKA | 2118 | GQDSPHKSG | 2228 |
| QQ | | HQ | | QNPQ | | QYQQ | |
| GSGSPHSKAQHP | 1899 | GSGSPHSKAGD | 2009 | GHDSPHKSG | 2119 | GHDSPHKSG | 2229 |
| S | | SQ | | RNQE | | QQIM | |
| GLGSPHSKSQN | 1900 | GSGSPHSKLKSQ | 2010 | GHDSPHKSG | 2120 | GHDSPHKSR | 2230 |
| QQ | | 0 | | QTQL | | QNEQ | |
| GTGSPHSKAQN | 1901 | GSGSPHSKAQKI | 2011 | GHDSPHKSE | 2121 | GHDSPHKSG | 2231 |
| QQ | | S | | KNQQ | | LNHQ | |
| GSGSPHSKAPGL | 1902 | GSGSPHSKAPS | 2012 | GRDSPHKSG | 2122 | GYDSPHKSG | 2232 |
| Q | | MQ | | QDQQ | | QNQQ | |
| GSGSPHSKAQGI | 1903 | GSGSPHSKASPR | 2013 | GHDSPHKTG | 2123 | GHDSPHKSG | 2233 |
| R | | Q | | HNQQ | | QNLQ | |
| GSGSPHSKAQAP | 1904 | GSGSPHSKRME | 2014 | GYDSPHKSG | 2124 | GHDSPHKSR | 2234 |
| A | | QQ | | QTQQ | | QDQQ | |
| GSGSPHSKSQSQ | 1905 | GSGSPHSKAQY | 2015 | GHESPHKSG | 2125 | GDDSPHKSG | 2235 |
| Q | | QN | | QTQQ | | QKQL | |
| GSGSPHSKAQIP | 1906 | GSGSPHARMQN | 2016 | GHDSPHKSG | 2126 | GSGSPHSKA | 2236 |
| P | | QQ | | QSKQ | | QNQA | |
| GSGSPHSKAQT | 1907 | GSGSPHVKSQN | 2017 | GHDSPHKTG | 2127 | GDDSPHKSG | 2237 |
| QL | | QQ | | QNQP | | HNQQ | |
| GSGSPHSKAQAP | 1908 | GQDSPHKSGQN | 2018 | GHDSPHKSG | 2128 | GHDSPHKSG | 2238 |
| S | | QQ | | QSPQ | | QMIH | |
| GHDSPHKSGRN | 2239 | GHDSPHKSVQN | 2240 | GHDSPHKSG | 2241 | TINGHDSPH | 2728 |
| HQ | | RQ | | QKMN | | KSRLNQP | |
| TINGSGSPHSKA | 2242 | TDRGSGSPHSK | 2404 | TINGSGSPHS | 2566 | TVDGHDSPH | 2729 |
| QNQQ | | AQNQQ | | KAQSTM | | KSGQKQQ | |
| TINGHDSPHKSG | 2243 | TINGSGSPHSKA | 2405 | TVNASGSPHS | 2567 | TINGQDSPH | 2730 |
| QNQQ | | QIPP | | KAQNQL | | KSGQNQD | |
| TIIGSGSPHSKAQ | 2244 | TVKGSGSPHSK | 2406 | TINGSGSPHS | 2568 | TIEGHDSPHK | 2731 |
| NRH | | AQDQQ | | KAQREM | | SGRNQQ | |
| TFPGSGSPHSKV | 2245 | NADGSGSPHSK | 2407 | TVHGSGSPHS | 2569 | TTNGHDSPH | 2732 |
| QNQQ | | AQNQQ | | KAQSQQ | | KSGQNLL | |
| TEKMSGSPHSK | 2246 | TDKVSGSPHSK | 2408 | TINGGGSPHS | 2570 | TINGHDSPH | 2733 |
| AQNQQ | | AQNQQ | | KSQNRQ | | KSGQLVI | |
| EINGRGSPHSKA | 2247 | TITGSGSPHSKA | 2409 | TINGSGSPHS | 2571 | TVNGHDSPH | 2734 |
| QNQQ | | QTQL | | KAQYRA | | KSRQSQQ | |
| TVNRNGSPHSK | 2248 | TINGSGSPHSKA | 2410 | TINGGGSPHS | 2572 | TINGHDSPH | 2735 |
| AQNQQ | | QAPS | | KAQRQQ | | KSGRTQE | |
| TVNGSGSPHSK | 2249 | NCVGSGSPHSK | 2411 | TEPMSGSPHS | 2573 | TINGHDSPH | 2736 |
| ARDQQ | | AQNQQ | | KAQNQQ | | KSVQTHQ | |
| TFNGSGSPHSKA | 2250 | TIRDAGSPHSKA | 2412 | TINGSGSPHS | 2574 | TSNGHDSPH | 2737 |
| PNLQ | | QNQQ | | KNQWQQ | | KSGQNQP | |
| TEKTSGSPHSKA | 2251 | TVKDSGSPHSK | 2413 | ETAGSGSPHS | 2575 | VINGHDSPH | 2738 |
| QNQQ | | AQNQQ | | KAQNQQ | | KSGQTQQ | |
| TINGSGSPHSKA | 2252 | NALGSGSPHSK | 2414 | TINGSGSPHS | 2576 | TINGPDSPHK | 2739 |
| HVRQ | | AQNQQ | | KAQRMN | | IGQNQQ | |
| TVNGSGSPHSK | 2253 | VINGSGSPHSKG | 2415 | NNLGSGSPHS | 2577 | AVNGHDSPH | 2740 |
| APNQH | | QNQQ | | KAQNQQ | | KSVQNQQ | |
| TEKISGSPHSKA | 2254 | TVNGGGSPHSK | 2416 | TINGSGSPHA | 2578 | TINGHDSPH | 2741 |
| QNQQ | | AQNQQ | | KAQNHQ | | KSRQDQH | |
| TINGPGSPHSKA | 2255 | TIQDGGSPHSKA | 2417 | TIIKNGSPHS | 2579 | AINGPDSPHK | 2742 |
| HNQQ | | QNQQ | | KAQNQQ | | SGQKQQ | |
| TVNGSGSPHSKT | 2256 | TISGGGSPHSKA | 2418 | TINGSGSPHS | 2580 | TINGHDSPH | 2743 |
| QSQQ | | QNQQ | | KAGDSQ | | KSRQSQH | |
| SINESGSPHSKA | 2257 | TSNASGSPHSKA | 2419 | TINGSGSPHS | 2581 | TIYGHDSPH | 2744 |
| QNQQ | | HNQQ | | KLKSQQ | | KSVQNQL | |
| TERTSGSPHSKA | 2258 | TINGSGSPHSKA | 2420 | TINGSGSPHS | 2582 | TVNGHDSPH | 2745 |
| QNQQ | | QNTY | | KAQKIS | | KSGQNLL | |
| TINGSGSPHSKA | 2259 | TINGSGSPHSKS | 2421 | TEYNSGSPHS | 2583 | TENKSGSPHS | 2746 |
| QPAK | | QNQH | | KAQNQQ | | KAQNQQ | |
| TEKSSGSPHSKA | 2260 | TINGGGSPHSKA | 2422 | TINGSGSPHS | 2584 | TTNGQDSPH | 2747 |
| QNQQ | | QDKQ | | KAPSMQ | | KSGQNQQ | |
| TSYGNGSPHSK | 2261 | TEFVSGSPHSKA | 2423 | AINGSGSPHS | 2585 | TDKGSGSPH | 2748 |
| AQNQQ | | QNQQ | | KAQNQQ | | SKAQNQQ | |
| TEKGSGSPHSKA | 2262 | TVNGSGSPHSK | 2424 | TINGSGSPHS | 2586 | TIDGHDSPH | 2749 |
| QNQQ | | AQNHL | | KASPRQ | | KSGRNQQ | |
| TINGSGSPHSKS | 2263 | TREISGSPHSKA | 2425 | TINGSGSPHS | 2587 | TINGYDSPH | 2750 |
| QTQQ | | QNQQ | | KRMEQQ | | KSGQYQH | |
| TERISGSPHSKA | 2264 | TINGSGSPHSKA | 2426 | TINGSGSPHS | 2588 | TDNGHDSPH | 2751 |
| QNQQ | | QIGM | | KAQYQN | | KSRQNQQ | |
| TERASGSPHSKA | 2265 | TIDGSGSPHSKA | 2427 | TINGSGSPHS | 2589 | TINGHDSPH | 2752 |
| QNQQ | | LNKQ | | KAQYYV | | KSWVRQQ | |
| ELHGSGSPHSKA | 2266 | TIIGGGSPHSKA | 2428 | TINGSGSPHS | 2590 | TINGHESPHK | 2753 |
| QNQQ | | QNPQ | | KLRRQQ | | SGQNQH | |
| AINGSGSPHSKA | 2267 | QGEGSGSPHSK | 2429 | TINGSGSPHS | 2591 | TVNGHDSPH | 2754 |
| QNLA | | AQNQQ | | KAGCGQ | | KIGHNQQ | |
| TVNGSGSPHSKS | 2268 | TINGTGSPHSKA | 2430 | SMNGSGSPH | 2592 | TCNGHDSPH | 2755 |
| QNQL | | PNQL | | SRAQNQQ | | KSGRNQQ | |
| TERNSGSPHSKA | 2269 | TVNGSGSPHSK | 2431 | TINGSGSPHS | 2593 | TINGNGSPHS | 2756 |
| QNQQ | | AQLQQ | | KRLRQQ | | KAQNHQ | |
| SVNGNGSPHSK | 2270 | TENGGGSPHSK | 2432 | TINGSGSPHS | 2594 | NVVGHDSPH | 2757 |
| AQNQQ | | AQYQQ | | LRRNQQ | | KSGQNQQ | |
| TFNGSGSPHSKA | 2271 | SINGSGSPHSKT | 2433 | TINGSGSPHS | 2595 | TINGHDSPH | 2758 |
| QGQQ | | QSQQ | | RGRNQQ | | KSNAWQQ | |
| TERVSGSPHSKA | 2272 | TVNGGGSPHSK | 2434 | TINGSGSPHS | 2596 | TDAGHDSPH | 2759 |
| QNQQ | | AQHQQ | | SRRNQQ | | KSGQNQQ | |
| TINGSGSPHSKA | 2273 | SEKGSGSPHSKA | 2435 | TINGSGSPHS | 2597 | TEVGHDSPH | 2760 |
| LNRQ | | QNQQ | | KAFRLQ | | KSGQNQQ | |
| TERLSGSPHSKA | 2274 | NVNGSGSPHSK | 2436 | TINGSCSPHR | 2598 | SELGHDSPH | 2761 |
| QNQQ | | AQNQQ | | KAQNQQ | | KSGQNQQ | |
| TDNGSGSPHSK | 2275 | GGEGSGSPHSK | 2437 | TINGSGSPHF | 2599 | TINGHDSPH | 2762 |
| AHNQQ | | AQNQQ | | LRQNQQ | | KSGQSVP | |
| TFHGSGSPHSKT | 2276 | TINGSGSPHSKA | 2438 | TINGSGSPHS | 2600 | TINGHESPHK | 2763 |
| QNQQ | | QRMS | | LRFNQQ | | SGQNIQ | |
| TINGGGSPHSKA | 2277 | TINGSGSPHSKA | 2439 | TINGSGSPHS | 2601 | TINGHDSPH | 2764 |
| QTQQ | | QGIL | | KWLLQQ | | KSVQNHL | |
| TSNGSGSPHSKA | 2278 | EFVGSGSPHSKA | 2440 | TINGSGSPHS | 2602 | TINGHDSPH | 2765 |
| QNPP | | QNQQ | | KRRLQQ | | KIGLDQQ | |
| TINGSGSPHSKA | 2279 | TIIGSGSPHSKAQ | 2441 | TINGSGSPHS | 2603 | TSNASGSPHS | 2766 |
| QNLQ | | DRQ | | KAQRKL | | KAQHQQ | |
| TVHGNGSPHSK | 2280 | SDKGSGSPHSK | 2442 | TINGSGSPHS | 2604 | TINGHDSPH | 2767 |
| AQNQQ | | AQNQQ | | KALRRQ | | KRGPDQQ | |
| TINGGGSPHSKA | 2281 | TEQVSGSPHSKA | 2443 | TINGSGSPHS | 2605 | TINGMGSPH | 2768 |
| QNQQ | | QNQQ | | KAQRLR | | SKTQNQQ | |
| TENMSGSPHSK | 2282 | TEHVSGSPHSKA | 2444 | YLSGSGSPHS | 2606 | TIKGHDSPH | 2769 |
| AQNQQ | | QNQQ | | KAQNQQ | | KSGESQQ | |
| TENVSGSPHSKA | 2283 | TINGSGSPHSKA | 2445 | TINGSGSPHS | 2607 | TINGHDSPH | 2770 |
| QNQQ | | RDWQ | | KAQRRL | | KHGQNHQ | |
| TSSGSGSPHSKA | 2284 | TENASGSPHSKA | 2446 | TINGSGSPHS | 2608 | TVNGTGSPH | 2771 |
| QYQQ | | QNQQ | | KARRQQ | | SKAQNQL | |
| TIDGGGSPHSKA | 2285 | EVQGSGSPHSK | 2447 | TINGSGSPHS | 2609 | TIIGHDSPHK | 2772 |
| QNKQ | | AQNQQ | | KARRLQ | | SGQYQH | |
| TEKVSGSPHSKA | 2286 | TINGSGSPHSKA | 2448 | TINGSGSPHS | 2610 | TSNGHDSPH | 2773 |
| QNQQ | | QNTH | | KSRRQQ | | KSVQNKQ | |
| AINGSGSPHSKA | 2287 | TINGSGSPHSKA | 2449 | TINGLLSPHW | 2611 | IVNGQVSPH | 2774 |
| QDQE | | PNLQ | | KAQNQQ | | KSGQNQQ | |
| TCNKSGSPHSKA | 2288 | TINGSGSPHSKA | 2450 | TINGSGSPHS | 2612 | TVNGHDSPH | 2775 |
| QNQQ | | QERS | | KARLRQ | | KSGQRQL | |
| TINGGGSPHSKA | 2289 | TSNGSGSPHSKA | 2451 | TINGSGSPHS | 2613 | TVNGHDSPH | 2776 |
| QNQL | | QNYQ | | KASKRQ | | KIGQNQL | |
| NINGGGSPHSKA | 2290 | TEYISGSPHSKA | 2452 | TINGSGSPHV | 2614 | TINGHDSPH | 2777 |
| QNQQ | | QNQQ | | RRQNQQ | | KSGQIIV | |
| TEHLSGSPHSKA | 2291 | TINGSGSPHSKA | 2453 | TINGSGSPHS | 2615 | IGNGHESPH | 2778 |
| QNQQ | | QRTC | | KAQLYR | | KSGQNQQ | |
| AEMGSGSPHSK | 2292 | TINGSGSPHSKA | 2454 | GLSGSGSPHS | 2616 | EVMGHDSPH | 2779 |
| AQNQQ | | QIGH | | KAQNQQ | | KSGQNQQ | |
| ATNGSGSPHSK | 2293 | NCWGSGSPHSK | 2455 | TINGSGSPHS | 2617 | TINGYDSPH | 2780 |
| AQNHQ | | AQNQQ | | KAQLTV | | KSGQKQS | |
| AIKGSGSPHSKA | 2294 | TINGSGSPHSKA | 2456 | TINGHDSPHK | 2618 | TIHGNGSPHS | 2781 |
| QDQQ | | QGAI | | RGQHRQ | | KAQNQE | |
| TINGGGSPHSKS | 2295 | TDVNSGSPHSK | 2457 | MPEGHDSPH | 2619 | YQVGHDSPH | 2782 |
| QNQL | | AQNQQ | | KSGQNQQ | | KSGQNQQ | |
| TVNGNGSPHSK | 2296 | SDIGSGSPHSKA | 2458 | MEGGHDSPH | 2620 | TIKGDDSPH | 2783 |
| AQNKQ | | QNQQ | | KSGQNQQ | | KSVQNQQ | |
| TINGSGSPHSKG | 2297 | TINGSGSPHSKA | 2459 | MEYGHDSPH | 2621 | TINGHDSPH | 2784 |
| HWQQ | | QVPP | | KSGQNQQ | | KSVQSHQ | |
| TDKTSGSPHSKA | 2298 | TINGSGSPHSKA | 2460 | AEWGHDSPH | 2622 | TINGHDSPH | 2785 |
| QNQQ | | QVQQ | | KSGQNQQ | | KSGQFVV | |
| TFKGSGSPHSKA | 2299 | TINGSGSPHSKA | 2461 | CEWGHDSPH | 2623 | TVNGHDSPH | 2786 |
| PNQQ | | LMRQ | | KSGQNQQ | | KSRQNLQ | |
| TVNGSGSPHSK | 2300 | TINGSGSPHSKA | 2462 | ANNGQDSPH | 2624 | ATNGHNSPH | 2787 |
| AQNQL | | QYSV | | KSGQNQQ | | KSGQNQE | |
| TINGSGSPHSKR | 2301 | NSIGSGSPHSKA | 2463 | IPEGHDSPHK | 2625 | AINGHDSPH | 2788 |
| PEQQ | | QNQQ | | SGQNQQ | | KSAQNQQ | |
| TINGSGSPHSKA | 2302 | TINGSGSPHSKV | 2464 | ADMGHDSPH | 2626 | TEHGHDSPH | 2789 |
| QRTM | | PNLQ | | KSGQNQQ | | KSGQNQQ | |
| TEKASGSPHSKA | 2303 | AINGSGSPHSKA | 2465 | IEYGHDSPHK | 2627 | TIY GHDSPH | 2790 |
| QNQQ | | QSQQ | | SGQNQQ | | KSGQSQP | |
| SDQGSGSPHSKA | 2304 | TINGSGSPHSKA | 2466 | ADYGHDSPH | 2628 | TISGHESPHK | 2791 |
| QNQQ | | QAIT | | KSGQNQQ | | SGQNEQ | |
| TEITSGSPHSKA | 2305 | TINGSGSPHSKA | 2467 | IETGHDSPHK | 2629 | AIIGHDSPHK | 2792 |
| QNQQ | | QKTL | | SGQNQQ | | SAQNQQ | |
| TDKSSGSPHSKA | 2306 | TVNGSGSPHSK | 2468 | MEWGHDSPH | 2630 | AIDGHDSPH | 2793 |
| QNQQ | | AQNQW | | KSGQNQQ | | KSGQNQL | |
| TIDGSGSPHSKA | 2307 | TINGSGSPHSKA | 2469 | CEYGHDSPH | 2631 | TIMGHDSPH | 2794 |
| QNQQ | | QLHH | | KSGQNQQ | | KSVQNQQ | |
| TVNGNGSPHSK | 2308 | TEQTSGSPHSKA | 2470 | RINGHDSPHK | 2632 | EVGGHDSPH | 2795 |
| AQNQH | | QNQQ | | SGQKQQ | | KSGQNQQ | |
| NTNGSGSPHSK | 2309 | TINGSGSPHSKA | 2471 | MEIGHDSPH | 2633 | TINGHDSPH | 2796 |
| AQNQQ | | QNII | | KSGQNQQ | | KSAQNLL | |
| TETHSGSPHSKA | 2310 | NSLGSGSPHSKA | 2472 | LEYGHDSPH | 2634 | TINASGSPHS | 2797 |
| QNQQ | | QNQQ | | KSGQNQQ | | KAINQQ | |
| TINGGGSPHSKA | 2311 | TIPMEGSPHSKA | 2473 | ADWGHDSPH | 2635 | AINGNGSPH | 2798 |
| LNQQ | | QNQQ | | KSGQNQQ | | KRGQNQQ | |
| TINGSGSPHSKA | 2312 | TINGSGSPHSKA | 2474 | IEIGHDSPHK | 2636 | SEMGHDSPH | 2799 |
| LHQH | | QGHH | | SGQNQQ | | KSGQNQQ | |
| TINGTGSPHSKA | 2313 | TDRTSGSPHSKA | 2475 | DIMGHDSPH | 2637 | AQQGHDSPH | 2800 |
| QNHQ | | QNQQ | | KSGQNQQ | | KSGQNQQ | |
| TINGSGSPHSKA | 2314 | TINGSGSPHSKA | 2476 | FEQGHDSPH | 2638 | AINGHDSPH | 2801 |
| QHRI | | QSKV | | KSGQNQQ | | KSGQSLQ | |
| TINGSGSPHSKA | 2315 | EVVGSGSPHSK | 2477 | MEFGHDSPH | 2639 | TINGSGSPHS | 2802 |
| QYIH | | AQNQQ | | KSGQNQQ | | KAPNQQ | |
| TENISGSPHSKA | 2316 | TINGSGSPHSKA | 2478 | CDQGHDSPH | 2640 | CGEGHDSPH | 2803 |
| QNQQ | | QLPS | | KSGQNQQ | | KSGQNQQ | |
| TIIGGGSPHSKA | 2317 | TINGSGSPHSKAI | 2479 | LPEGHDSPH | 2641 | TVNGHDSPH | 2804 |
| HNQQ | | GKQ | | KSGQNQQ | | KSAQNHQ | |
| TINGSGSPHSKA | 2318 | TEPTSGSPHSKA | 2480 | IENGHDSPHK | 2642 | TVNGHDSPH | 2805 |
| QKFE | | QNQQ | | SGQNQQ | | KSGQTQL | |
| TSNESGSPHSKA | 2319 | TVNGGGSPHSK | 2481 | MESGHDSPH | 2643 | TNNGHDSPH | 2806 |
| QNHQ | | SQNQQ | | KSGQNQQ | | KSGRNRQ | |
| TINGSGSPHSKA | 2320 | TINGSGSPHSKA | 2482 | AEIGHDSPHK | 2644 | TINGHDSPH | 2807 |
| QFPS | | QAIH | | SGQNQQ | | KYGQNEQ | |
| TERPSGSPHSKA | 2321 | TINGSGSPHSKA | 2483 | VEYGHDSPH | 2645 | TINGNGSPHS | 2808 |
| QNQQ | | QHGL | | KSGQNQQ | | KAPNLQ | |
| TINGNGSPHSKA | 2322 | SELGSGSPHSKA | 2484 | IINGHDSPHK | 2646 | SINGHDSPHK | 2809 |
| QNPL | | QNQQ | | SGLTQQ | | SQQNQQ | |
| SIKGNGSPHSKA | 2323 | TINGSGSPHSKA | 2485 | TSNGDDSPH | 2647 | TIGGHDSPH | 2810 |
| QNQQ | | QFMC | | KSGRNQQ | | KSGQNQQ | |
| TERMSGSPHSK | 2324 | TINVSGSPHSKA | 2486 | IEVGHDSPHK | 2648 | TINGHDSPH | 2811 |
| AQNQQ | | QGQQ | | SGQNQQ | | KSVQSKQ | |
| TERSSGSPHSKA | 2325 | TINGGGSPHSKA | 2487 | MEMGHDSPH | 2649 | ELVGHDSPH | 2812 |
| QNQQ | | QNQM | | KSGQNQQ | | KSGQNQQ | |
| TELHSGSPHSKA | 2326 | TVNGSGSPHSK | 2488 | AEVGHDSPH | 2650 | ELMGHDSPH | 2813 |
| QNQQ | | AQHLQ | | KSGQNQQ | | KSGQNQQ | |
| TELTSGSPHSKA | 2327 | TIRENGSPHSKA | 2489 | MDAGHDSPH | 2651 | TINGNDSPH | 2814 |
| QNQQ | | QNQQ | | KSGQNQQ | | KIGHNQQ | |
| TINGSGSPHSKA | 2328 | TINGSGSPHSKT | 2490 | VEWGHDSPH | 2652 | TIKGGGSPHS | 2815 |
| HNQQ | | QNHQ | | KSGQNQQ | | KAQDQQ | |
| TINGGGSPHSKA | 2329 | TINGSGSPHSKA | 2491 | AEQGHDSPH | 2653 | TVNGHDSPH | 2816 |
| QSQQ | | QPAR | | KSGQNQQ | | KSGQTQQ | |
| TINGSGSPHSKA | 2330 | TVNGSGSPHSK | 2492 | LEWGHDSPH | 2654 | TINGQDSPH | 2817 |
| QAIK | | AQSLQ | | KSGQNQQ | | KSGQNPL | |
| TENTSGSPHSKA | 2331 | TINGSGSPHSKS | 2493 | MELGHDSPH | 2655 | TVNASGSPH | 2818 |
| QNQQ | | QSQL | | KSGQNQQ | | SKAQNHQ | |
| TIDGSGSPHSKG | 2332 | TINGSASPHSKA | 2494 | METGHDSPH | 2656 | TINGHDSPH | 2819 |
| QNRQ | | HSQQ | | KSGQNQQ | | KSGRDQK | |
| NINGSGSPHSKA | 2333 | TWQNSGSPHSK | 2495 | MEAGHDSPH | 2657 | TINGHDSPH | 2820 |
| QSQQ | | AQNQQ | | KSGQNQQ | | KSVHNQQ | |
| TINGSVSPHGKA | 2334 | TINGSGSPHSKA | 2496 | IESGHDSPHK | 2658 | TINGHDSPH | 2821 |
| QNQL | | QDRQ | | SGQNQQ | | KSGQWKR | |
| TSNASGSPHSKA | 2335 | TINGSGSPHSKA | 2497 | MEVGHDSPH | 2659 | TIDGSGSPHS | 2822 |
| QNQL | | QMPS | | KSGQNQQ | | KAENRQ | |
| TEARSGSPHSKA | 2336 | TNNGGGSPHSK | 2498 | CEIGHDSPHK | 2660 | NEIGHDSPH | 2823 |
| QNQQ | | AQNLQ | | SGQNQQ | | KSGQNQQ | |
| TEKNSGSPHSKA | 2337 | TINGSGSPHSKA | 2499 | ATNGHDSPH | 2661 | AINGHDSPH | 2824 |
| QNQQ | | QGSL | | KSGLNQQ | | KSGQSQQ | |
| TANGSGSPHSK | 2338 | TEVTSGSPHSKA | 2500 | MDGGHDSPH | 2662 | IINGHDSPHK | 2825 |
| AQYQQ | | QNQQ | | KSGQNQQ | | SRQAQQ | |
| TVNGSGSPHSK | 2339 | SINGGGSPHSKA | 2501 | QEVGHDSPH | 2663 | TPNGHDSPH | 2826 |
| AQYQH | | QYQQ | | KSGQNQQ | | KSGQNQQ | |
| TINGSGSPHTKA | 2340 | TVIGSGSPHSKS | 2502 | ADQGHDSPH | 2664 | ITNGHDSPH | 2827 |
| QNPQ | | QNQQ | | KSGQNQQ | | KSGQTQQ | |
| TINGSGSPHSKG | 2341 | AVNVSGSPHSK | 2503 | TINGHESPHK | 2665 | TINGHDSPH | 2828 |
| QNPP | | AQNQQ | | SAQNHQ | | KSVQNHQ | |
| TIIGSGSPHSKAQ | 2342 | TVNGNGSPHSK | 2504 | TINGHDSPHK | 2666 | TINGHDSPH | 2829 |
| HQL | | SQNQQ | | SAQNQW | | KSKQNQQ | |
| TINGSGSPHSKA | 2343 | TDRNSGSPHSK | 2505 | NMNGHDSPH | 2667 | TINGHDSPH | 2830 |
| QSPP | | AQNQQ | | KSGQNTH | | KSAQNQL | |
| TIYGSGSPHSKA | 2344 | TINGSGSPHSKA | 2506 | IEMGHDSPH | 2668 | TVNGHDSPH | 2831 |
| QNQQ | | QVPA | | KSGQNQQ | | KSGQTQP | |
| TINGSGSPHSKA | 2345 | GVLGSGSPHSK | 2507 | TINGHDSPHK | 2669 | TDQGHDSPH | 2832 |
| QAKL | | AQNQQ | | SGRRRQ | | KSGQNQQ | |
| TDKNSGSPHSK | 2346 | TLNGNGSPHSK | 2508 | ISNGHDSPHK | 2670 | TINGHDSPH | 2833 |
| AQNQQ | | AQNLQ | | SAQNQQ | | KLWINQQ | |
| TINGSGSPHSKT | 2347 | AINGSGSPHSKA | 2509 | TGNGHDSPH | 2671 | GINGPDSPHK | 2834 |
| KSQQ | | QDKQ | | KSGQYQQ | | SGQNQQ | |
| TINGSGSPHSKA | 2348 | TSNGSGSPHSKA | 2510 | TINGNYSPHK | 2672 | SEIGHDSPHK | 2835 |
| QDRP | | HYQQ | | IGQNQQ | | SGQNQQ | |
| TINGIGSPHSKA | 2349 | TINGSGSPHSKA | 2511 | TINGHDSPHK | 2673 | TINGHDSPH | 2836 |
| QNLG | | QVPS | | SRQNDQ | | KSVQKQL | |
| TINGSGSPHSKA | 2350 | TELRSGSPHSKA | 2512 | QQQGHDSPH | 2674 | TINGHPSPH | 2837 |
| QSQQ | | QNQQ | | KSGQNQQ | | WKGQNQQ | |
| TENLSGSPHSKA | 2351 | NINGSGSPHSKA | 2513 | HDWGHDSPH | 2675 | TVNGHDSPH | 2838 |
| QNQQ | | QNHQ | | KSGQNQQ | | KSGRNQL | |
| TINGSGSPHSKA | 2352 | TVNGGGSPHSK | 2514 | IEGGHDSPHK | 2676 | TIKGSGSPHS | 2839 |
| QAFH | | AQNHQ | | SGQNQQ | | KVQDQQ | |
| TINGSGSPHSKA | 2353 | TINGSGSPHSKA | 2515 | TFNRSGSPHS | 2677 | SEKGHDSPH | 2840 |
| QKQQ | | RGEQ | | KAQNQQ | | KSGQNQQ | |
| TFSGSGSPHSKA | 2354 | TINGGGSPHSKA | 2516 | AINGHDSPH | 2678 | WSAGHDSPH | 2841 |
| QNLQ | | QYQH | | KSGQIRQ | | KSGQNQQ | |
| AINGSGSPHSKA | 2355 | TEDLSGSPHSKA | 2517 | TINGHDSPHK | 2679 | ELAGHDSPH | 2842 |
| QNAQ | | QNQQ | | IGQNQH | | KSGQNQQ | |
| TESWSGSPHSKA | 2356 | TINGSGSPHSKA | 2518 | AINGYDSPH | 2680 | TINGHDSPH | 2843 |
| QNQQ | | PGQQ | | KSGQKQQ | | KMGRNQQ | |
| TTNGSGSPHSKA | 2357 | TIPKNGSPHSKA | 2519 | TESGHDSPH | 2681 | TINGHDSPH | 2844 |
| HNQL | | QNQQ | | KSGQNQQ | | KSGISIQ | |
| TVNGNGSPHSK | 2358 | TINGSGSPHSKA | 2520 | TINGHDSPHK | 2682 | TSNGHDSPH | 2845 |
| AQNHQ | | QSLQ | | SGQSVQ | | KSVQNLQ | |
| TEDKSGSPHSKA | 2359 | TINGSGSPHSKR | 2521 | TINGHESPHK | 2683 | QTQGHDSPH | 2846 |
| QNQQ | | LEQQ | | SGRSQQ | | KSGQNQQ | |
| TESASGSPHSKA | 2360 | TERGSGSPHSKA | 2522 | TINGHDSPHK | 2684 | TINGHDSPH | 2847 |
| QNQQ | | QNQQ | | SGQNKL | | KMAHNQQ | |
| TNNGSGSPHSK | 2361 | TVNGSGSPHSK | 2523 | TINGHDSPHK | 2685 | AINGSGSPHS | 2848 |
| AQNQQ | | APNQQ | | TGQNQQ | | KAQTQQ | |
| TSNGGGSPHSK | 2362 | TSNGSGSPHSKA | 2524 | TINGRGSPHK | 2686 | TINGHDSPH | 2849 |
| AQNLQ | | QNQS | | RGQNQQ | | KHGQNQQ | |
| TDKMSGSPHSK | 2363 | TINGSGSPHSKA | 2525 | TINGSGSPHT | 2687 | GADGHDSPH | 2850 |
| AQNQQ | | QKVI | | KAQNPP | | KSGQNQQ | |
| EVHGSGSPHSK | 2364 | TEGISGSPHSKA | 2526 | TINGQDSPHK | 2688 | VGEGHDSPH | 2851 |
| AQNQQ | | QNQQ | | SGQHQQ | | KSGQNQQ | |
| TINGSGSPHSKA | 2365 | TINGSGSPHSKA | 2527 | SINGHDSPHK | 2689 | ANEGHDSPH | 2852 |
| QKLN | | QNND | | SGQIQH | | KSGQNQQ | |
| TINGGGSPHSKS | 2366 | TINGSGSPHSKA | 2528 | AINGHDSPH | 2690 | TEAKSGSPHS | 2853 |
| QNQH | | QSVH | | KSGPRQQ | | KAQNQQ | |
| TVNGGGSPHSK | 2367 | TINGSGSPHSKA | 2529 | TVNGHDSPH | 2691 | TINGHDSPH | 2854 |
| AQSQQ | | QPLG | | KSGHTQQ | | KSVQSQQ | |
| TTNGSGSPHSKA | 2368 | TINKEGSPHSKA | 2530 | SINGHDSPHK | 2692 | TIPGSGSPHS | 2855 |
| QYQH | | QNQQ | | SGQRQH | | KAQNLQ | |
| TISGSGSPHSKA | 2369 | TCNASGSPHSK | 2531 | SLNGSGSPHT | 2693 | TINGHDSPH | 2856 |
| QYQH | | AQNQQ | | KAQNQQ | | KSGQTVC | |
| TESTSGSPHSKA | 2370 | AINGSGSPHSKA | 2532 | AINGHDSPH | 2694 | ELRGHDSPH | 2857 |
| QNQQ | | HNQE | | KSAQSQQ | | KSGQNQQ | |
| TINGSGSPHSKS | 2371 | TEGLSGSPHSKA | 2533 | SIYGHESPHK | 2695 | CQIGHDSPH | 2858 |
| QNVQ | | QNQQ | | SGQNQQ | | KSGQNQQ | |
| SINGSGSPHSKA | 2372 | TRDASGSPHSK | 2534 | TVNGHDSPH | 2696 | GVMGHDSPH | 2859 |
| QAQQ | | AQNQQ | | KSLQNQQ | | KSGQNQQ | |
| TVNGSGSPHSK | 2373 | TSNGSGSPHSKA | 2535 | TINGHGSPHS | 2697 | ACDGHDSPH | 2860 |
| AQNLQ | | QNLQ | | KAQNPQ | | KSGQNQQ | |
| TVRDSGSPHSKA | 2374 | TGNGSGSPHSK | 2536 | TSNGYDSPH | 2698 | TINGQDSPH | 2861 |
| QNQQ | | AQIQQ | | KSGQKQQ | | KSGQYQQ | |
| TFNASGSPHSKA | 2375 | TVNGGGSPHSK | 2537 | TVNGHDSPH | 2699 | TINGHDSPH | 2862 |
| PNQQ | | AQNLQ | | KSGRNQE | | KSGQQIM | |
| TDRMSGSPHSK | 2376 | TDRSSGSPHSKA | 2538 | TTNGHDSPH | 2700 | TINGHDSPH | 2863 |
| AQNQQ | | QNQQ | | KSGQTQL | | KSRQNEQ | |
| TINGSGSPHSKA | 2377 | TINGSGSPHSKA | 2539 | AINGHDSPH | 2701 | ASNGHDSPH | 2864 |
| QTPP | | QVRN | | KSEKNQQ | | KSGLNHQ | |
| TIKGSGSPHSKA | 2378 | TINGSGSPHSKA | 2540 | IINGRDSPHK | 2702 | TVNGHDSPH | 2865 |
| QNQQ | | PSNQ | | SGQDQQ | | KSGQSQP | |
| NHIGSGSPHSKA | 2379 | TINGSGSPHSKA | 2541 | TISGHDSPHK | 2703 | NELGHDSPH | 2866 |
| QNQQ | | QVGH | | TGHNQQ | | KSGQNQQ | |
| TINGSGSPHSKA | 2380 | NAIGSGSPHSKA | 2542 | SINGYDSPHK | 2704 | AAEGHDSPH | 2867 |
| QYQH | | QNQQ | | SGQTQQ | | KSGQNQQ | |
| TIPIDGSPHSKAQ | 2381 | AENGSGSPHSK | 2543 | TINGHESPHK | 2705 | GQNGHDSPH | 2868 |
| NQQ | | AQNQQ | | SGQTQQ | | KSGQNQQ | |
| TINGSGSPHSKA | 2382 | TINGSGSPHSKA | 2544 | TINGHDSPHK | 2706 | NEFGHDSPH | 2869 |
| QGQQ | | QRDI | | SGQSKQ | | KSGQNQQ | |
| TFNGSGSPHNK | 2383 | TINGSGSPHSKA | 2545 | AIIGHESPHK | 2707 | TSIGYDSPHK | 2870 |
| AQNHQ | | QMPN | | SGQNQQ | | SGQNQQ | |
| ESDGSGSPHSKA | 2384 | TVNGSGSPHSKS | 2546 | TINGHDSPHK | 2708 | TDNGHDSPH | 2871 |
| QNQQ | | QNQQ | | TGQNQP | | KSGQNLQ | |
| TINGSGSPHSKA | 2385 | TIPAIGSPHSKAQ | 2547 | AINGHDSPH | 2709 | TITGHDSPHK | 2872 |
| QPPA | | NQQ | | KSGQSPQ | | SRQDQQ | |
| TINGSGSPHSKA | 2386 | TINGSGSPHSKA | 2548 | TIKGNDSPHK | 2710 | AEHGHDSPH | 2873 |
| QERP | | RGLQ | | SVQNQQ | | KSGQNQQ | |
| TIKGSGSPHSKA | 2387 | TELGSGSPHSKA | 2549 | TEFGHDSPH | 2711 | TINGDDSPH | 2874 |
| QDLQ | | QNQQ | | KSGQNQQ | | KSGQKQL | |
| TDLKSGSPHSKA | 2388 | AETGSGSPHSKA | 2550 | TINGHDSPHK | 2712 | EILGHDSPHK | 2875 |
| QNQQ | | QNQQ | | SAQNYQ | | SGQNQQ | |
| TINGGGSPHSKA | 2389 | TINGSGSPHSKL | 2551 | TFNGSASPHS | 2713 | TIHGSGSPHS | 2876 |
| QNPP | | QKQQ | | KALNQQ | | KAQNQA | |
| TINGSGSPHSKA | 2390 | TINGSGSPHSKA | 2552 | TINGHESPHK | 2714 | AINGDDSPH | 2877 |
| QAMH | | PSLQ | | SAQNRQ | | KSGHNQQ | |
| TVPNSGSPHSKA | 2391 | TINGSGSPHSKA | 2553 | TTNGHDSPH | 2715 | TSNGHNSPH | 2878 |
| QNQQ | | QRDQ | | KSGQNQQ | | KSGQNQE | |
| TVIGSGSPHSKA | 2392 | TDVGSGSPHSK | 2554 | TIKGQDSPHK | 2716 | TINGHDSPH | 2879 |
| LNQQ | | AQNQQ | | IGQNQQ | | KSGQMIH | |
| TINGSGSPHSKA | 2393 | TINGSGSPHSKN | 2555 | TVNGHDSPH | 2717 | NAIGHDSPH | 2880 |
| QHPS | | RDQQ | | KSGQNHL | | KSGQNQQ | |
| TINGLGSPHSKS | 2394 | SINGSGSPHSKA | 2556 | SINGHDSPHK | 2718 | VINGHDSPH | 2881 |
| QNQQ | | PNLQ | | SGQYQH | | KSGRNHQ | |
| TINGTGSPHSKA | 2395 | TINGSGSPHSKA | 2557 | TINGNDSPHK | 2719 | TITGHDSPHK | 2882 |
| QNQQ | | QAKG | | SVQNHQ | | SVQNRQ | |
| TINGSGSPHSKA | 2396 | TVNGSGSPHSK | 2558 | TITGHDSPHK | 2720 | TINGHDSPH | 2883 |
| PGLQ | | AQDKQ | | SGQNQW | | KSGQKMN | |
| TINGSGSPHSKA | 2397 | TINGGGSPHSKA | 2559 | TNNGHDSPH | 2721 | TIHGHDSPH | 2884 |
| QGIR | | QNPQ | | KSVQNQH | | KSGQSQQ | |
| TESHSGSPHSKA | 2398 | TINGSGSPHSKA | 2560 | TIDGHDSPHK | 2722 | TEIGHDSPHK | 2885 |
| QNQQ | | QSAH | | SGQNQH | | SGQNQQ | |
| TINGSGSPHSKA | 2399 | TINGNGSPHSKS | 2561 | TVNGHDSPH | 2723 | TINGHDSPH | 2886 |
| QAPA | | QNQH | | KSGQTRQ | | KSGQYQH | |
| TINGSGSPHSKS | 2400 | TVPTSGSPHSKA | 2562 | TVNGHDSPH | 2724 | NCLGSGSPH | 2403 |
| QSQQ | | QNQQ | | KSGQNLH | | SKAQNQQ | |
| AEHGSGSPHSK | 2401 | TIDGSGSPHSKS | 2563 | AISGHDSPHK | 2725 | AINRSGSPHS | 2565 |
| AQNQQ | | QNHQ | | SGLNQQ | | KAQDQQ | |
| TEDRSGSPHSKA | 2402 | TDVKSGSPHSK | 2564 | AINGHDSPH | 2726 | TITGHDSPHK | 2727 |
| QNQQ | | AQNQQ | | KSAQNQE | | SGQHLQ | |
| GSGSPHSKAQN | 2887 | GSGSPHSKAQS | 2936 | GSGSPHSKA | 2985 | GSGSPHSKA | 3034 |
| RHT | | QQT | | QRMST | | QNNDQ | |
| GSGSPHSKVQN | 2888 | ASGSPHSKAQN | 2937 | GSGSPHSKA | 2986 | GSGSPHSKA | 3035 |
| QQT | | QLT | | QGILT | | QSVHT | |
| MSGSPHSKAQN | 2889 | RSGSPHSKAQN | 2938 | GSGSPHSKA | 2987 | GSGSPHSKA | 3036 |
| QQT | | QQT | | QDRQT | | QPLGT | |
| GSGSPHSKARD | 2890 | GSGSPHSKAQY | 2939 | GSGSPHSKA | 2988 | GSGSPHSKA | 3037 |
| QQT | | QHT | | RDWQT | | HNQET | |
| GSGSPHSKAPNL | 2891 | GSGSPHTKAQN | 2940 | GSGSPHSKA | 2989 | GSGSPHSKA | 3038 |
| QT | | PQS | | QNTHD | | QNLQI | |
| TSGSPHSKAQN | 2892 | GSGSPHSKGQN | 2941 | GSGSPHSKAP | 2990 | GSGSPHSKA | 3039 |
| QQT | | PPT | | NLQI | | QIQQT | |
| GSGSPHSKAHV | 2893 | GSGSPHSKAQH | 2942 | GSGSPHSKA | 2991 | GSGSPHSKA | 3040 |
| RQT | | QLT | | QERST | | QVRNT | |
| GSGSPHSKAPNQ | 2894 | GSGSPHSKAQSP | 2943 | GSGSPHSKA | 2992 | GSGSPHSKA | 3041 |
| HT | | PT | | QNYQT | | PSNQT | |
| ISGSPHSKAQNQ | 2895 | GSGSPHSKAQA | 2944 | GSGSPHSKA | 2993 | GSGSPHSKA | 3042 |
| QT | | KLT | | QRTCT | | QVGHT | |
| GSGSPHSKTQSQ | 2896 | GSGSPHSKTKSQ | 2945 | GSGSPHSKA | 2994 | GSGSPHSKA | 3043 |
| QT | | QT | | QIGHT | | QRDIT | |
| GSGSPHSKAQN | 3032 | GSGSPHSKAQD | 2946 | GSGSPHSKA | 2995 | GSGSPHSKA | 3044 |
| QST | | RPT | | QGAIT | | QMPNT | |
| ESGSPHSKAQN | 2898 | GSGSPHSKAQS | 2947 | GSGSPHSKA | 2996 | GSGSPHSKA | 3045 |
| QQI | | QQL | | QVPPT | | RGLQT | |
| GSGSPHSKAQPA | 2899 | GSGSPHSKAQA | 2948 | GSGSPHSKA | 2997 | GSGSPHSKL | 3046 |
| KT | | FHT | | QVQQI | | QKQQT | |
| SSGSPHSKAQNQ | 2900 | GSGSPHSKAQK | 2949 | GSGSPHSKAL | 2998 | GSGSPHSKA | 3047 |
| QT | | QQD | | MRQT | | PSLQT | |
| GSGSPHSKSQTQ | 2901 | GSGSPHSKAQN | 2950 | GSGSPHSKA | 2999 | GSGSPHSKA | 3048 |
| QN | | AQT | | QYSVT | | QRDQT | |
| ASGSPHSKAQN | 2902 | WSGSPHSKAQN | 2951 | GSGSPHSKVP | 3000 | GSGSPHSKN | 3049 |
| QQT | | QQT | | NLQT | | RDQQT | |
| GSGSPHSKAQN | 2903 | GSGSPHSKAHN | 2952 | GSGSPHSKA | 3001 | GSGSPHSKA | 3050 |
| LAT | | QLT | | QSQQI | | QAKGT | |
| GSGSPHSKSQNQ | 2904 | GSGSPHSKAQN | 2953 | GSGSPHSKA | 3002 | GSGSPHSKA | 3051 |
| LT | | QQY | | QAITT | | QSAHT | |
| NSGSPHSKAQN | 2905 | GSGSPHSKAQK | 2954 | GSGSPHSKA | 3003 | GSGSPHSKS | 3052 |
| QQT | | LNT | | QKTLT | | QNHQT | |
| GSGSPHSKAQG | 2906 | GSGSPHSKSQN | 2955 | GSGSPHSKA | 3004 | RSGSPHSKA | 3053 |
| QQT | | VQT | | QNQWT | | QDQQT | |
| VSGSPHSKAQN | 2907 | GSGSPHSKAQA | 2956 | GSGSPHSKA | 3005 | GSGSPHSKA | 3054 |
| QQT | | QQT | | QLHHT | | QSTMT | |
| GSGSPHSKALNR | 2908 | GSGSPHSKAQN | 2957 | GSGSPHSKA | 3006 | GSGSPHSKA | 3055 |
| QS | | LQA | | QNIII | | QREMT | |
| LSGSPHSKAQN | 2909 | DSGSPHSKAQN | 2958 | GSGSPHSKA | 3007 | GSGSPHSKA | 3056 |
| QQT | | QQT | | QGHHT | | QYRAT | |
| GSGSPHSKAHN | 2910 | ASGSPHSKAPN | 2959 | GSGSPHSKA | 3008 | GSGSPHSKN | 3057 |
| QQT | | QQT | | QSKVT | | QWQQT | |
| GSGSPHSKTQN | 2911 | GSGSPHSKAQTP | 2960 | GSGSPHSKA | 3009 | GSGSPHSKA | 3058 |
| QQT | | PT | | QLPST | | QRMNT | |
| GSGSPHSKAQNP | 2912 | GSGSPHSKAQY | 2961 | GSGSPHSKAI | 3010 | GSGSPHAKA | 3059 |
| PT | | QHA | | GKQT | | QNHQT | |
| GSGSPHSKAQN | 2913 | GSGSPHSKAQG | 2962 | GSGSPHSKA | 3011 | GSGSPHSKA | 3060 |
| LQT | | QQA | | QAIHT | | GDSQT | |
| GSGSPHSKAQY | 2914 | GSGSPHNKAQN | 2963 | GSGSPHSKA | 3012 | GSGSPHSKL | 3061 |
| QQT | | HQT | | QHGLT | | KSQQT | |
| GSGSPHSKAQD | 2915 | GSGSPHSKAQPP | 2964 | GSGSPHSKA | 3013 | GSGSPHSKA | 3062 |
| QET | | AT | | QFMCT | | QKIST | |
| KSGSPHSKAQN | 2916 | GSGSPHSKAQE | 2965 | VSGSPHSKA | 3014 | GSGSPHSKA | 3063 |
| QQT | | RPT | | QGQQT | | PSMQT | |
| GSGSPHSKAQN | 2917 | GSGSPHSKAQD | 2966 | GSGSPHSKA | 3015 | GSGSPHSKA | 3064 |
| HQT | | LQT | | QHLQT | | SPRQT | |
| GSGSPHSKAQD | 2918 | GSGSPHSKAQA | 2967 | GSGSPHSKTQ | 3016 | GSGSPHSKR | 3065 |
| QQT | | MHT | | NH?N | | MEQQT | |
| GSGSPHSKGHW | 2919 | GSGSPHSKALN | 2968 | GSGSPHSKA | 3017 | GSGSPHSKA | 3066 |
| QQT | | QQT | | QPART | | QYQNT | |
| GSGSPHSKAPNQ | 2920 | GSGSPHSKAQH | 2969 | GSGSPHSKA | 3018 | RSGSPHSKA | 3067 |
| QT | | PST | | QSLQT | | QNQQI | |
| GSGSPHSKAQN | 2921 | GSGSPHSKAPGL | 2970 | GSGSPHSKSQ | 3019 | GSGSPHTKA | 3068 |
| QLI | | QT | | SQLT | | QNPPT | |
| GSGSPHSKRPEQ | 2922 | GSGSPHSKAQGI | 2971 | GSGSPHSKA | 3020 | GSGSPHTKA | 3069 |
| QT | | RT | | QDRQS | | QNQQT | |
| GSGSPHSKAQRT | 2923 | GSGSPHSKAQA | 2972 | GSGSPHSKA | 3021 | ASGSPHSKA | 3070 |
| MT | | PAT | | QMPST | | QHQQT | |
| GSGSPHSKAQN | 2924 | GSGSPHSKSQSQ | 2973 | GSGSPHSKA | 3022 | ASGSPHSKAI | 3071 |
| QQH | | QI | | QGSLT | | NQQT | |
| HSGSPHSKAQN | 2925 | GSGSPHSKAQIP | 2974 | GSGSPHSKSQ | 3023 | GSGSPHSKA | 3072 |
| QQT | | PT | | NQQT | | PNQQH | |
| GSGSPHSKALH | 2926 | GSGSPHSKAQT | 2975 | GSGSPHSKA | 3024 | ASGSPHSKA | 3073 |
| QHT | | QLT | | QVPAT | | QNHQT | |
| GSGSPHSKAQH | 2927 | GSGSPHSKAQA | 2976 | GSGSPHSKA | 3025 | GSGSPHSKA | 3074 |
| RIT | | PST | | QDKQT | | ENRQT | |
| GSGSPHSKAQYI | 2928 | GSGSPHSKGQN | 2977 | GSGSPHSKA | 3026 | GSGSPHSKV | 3075 |
| HT | | QQT | | HYQQT | | QDQQT | |
| GSGSPHSKAQKF | 2929 | ASGSPHSKAHN | 2978 | GSGSPHSKA | 3027 | GSGSPHSKA | 3076 |
| ET | | QQT | | QVPST | | QTQQA | |
| ESGSPHSKAQN | 2930 | GSGSPHSKAQN | 2979 | GSGSPHSKA | 3028 | GSGSPHSKA | 3077 |
| HQT | | TYA | | RGEQT | | QNQAT | |
| GSGSPHSKAQFP | 2931 | GSGSPHSKSQN | 2980 | GSGSPHSKAP | 3029 | GSGSPHSKG | 2935 |
| ST | | QHI | | GQQT | | QNRQT | |
| PSGSPHSKAQNQ | 2932 | GSGSPHSKAQN | 2981 | GSGSPHSKA | 3030 | GSGSPHSKA | 2984 |
| QT | | HLT | | QSLQI | | QLQQT | |
| GSGSPHSKAHN | 2933 | GSGSPHSKAQIG | 2982 | GSGSPHSKRL | 3031 | GSGSPHSKA | 3033 |
| QQR | | MT | | EQQT | | QKVIT | |
| GSGSPHSKAQAI | 2934 | GSGSPHSKALN | 2983 | GHDSPHKHG | 3160 | GHDSPHKSG | 3202 |
| KT | | KQT | | QNHQT | | QWKRT | |
| GHDSPHKSGQIR | 3078 | GHDSPHKSGQN | 3119 | LHDSPHKSG | 3161 | GHDSPHKSG | 3203 |
| QT | | QHA | | QNQQT | | QSQQI | |
| GHDSPHKIGQN | 3079 | GHDSPHKSGQT | 3120 | GHDSPHKSV | 3162 | GHDSPHKSR | 3204 |
| QHA | | RQT | | QNKQT | | QAQQT | |
| GYDSPHKSGQK | 3080 | GHDSPHKSGQN | 3121 | GQVSPHKSG | 3163 | GHDSPHKSV | 3205 |
| QQT | | LHT | | QNQQT | | QNHQI | |
| GHDSPHKSGQN | 3081 | GHDSPHKSGLN | 3122 | GHDSPHKSG | 3164 | GHDSPHKSK | 3206 |
| QQT | | QQT | | QRQLT | | QNQQA | |
| GHDSPHKSGQS | 3082 | GHDSPHKSAQN | 3123 | GHDSPHKIG | 3165 | GHDSPHKSA | 3207 |
| VQT | | QET | | QNQLT | | QNQLN | |
| GHESPHKSGRSQ | 3083 | GHDSPHKSGQH | 3124 | GHDSPHKSG | 3166 | GHDSPHKSG | 3208 |
| QT | | LQT | | QIIVT | | QTQPT | |
| GHDSPHKSGQN | 3084 | GHDSPHKSRLN | 3125 | IHDSPHKSGQ | 3167 | FHDSPHKSG | 3209 |
| KLE | | QPT | | NQQT | | QNQQT | |
| GHDSPHKTGQN | 3085 | GHDSPHKSGQK | 3126 | GYDSPHKSG | 3168 | GHDSPHKLW | 3210 |
| QQK | | QQT | | QKQST | | INQQT | |
| GRGSPHKRGQN | 3086 | GQDSPHKSGQN | 3127 | MHDSPHKSG | 3169 | GPDSPHKSG | 3211 |
| QQT | | QDT | | QNQQT | | QNQQT | |
| GHDNPHKSGQN | 3087 | GHDSPHKSGRN | 3128 | GDDSPHKSV | 3170 | GHDSPHKSV | 3212 |
| QQT | | QQT | | QNQQT | | QKQLT | |
| GQDSPHKSGQH | 3088 | GHDSPHKSGQN | 3129 | GHDSPHKSV | 3171 | GHDSPHKSG | 3213 |
| QQA | | LLT | | QSHQT | | RNQLA | |
| GHDSPHKSGQIQ | 3089 | GHDSPHKSGQL | 3130 | GHDSPHKSG | 3172 | VHDSPHKSG | 3214 |
| HT | | VIT | | QFVVT | | QNQQS | |
| GHDSPHKSGPR | 3090 | GHDSPHKSRQS | 3131 | GHDSPHKSR | 3173 | GHDSPHKMG | 3215 |
| QQT | | QQT | | QNLQT | | RNQQS | |
| GHDSPHKSGHT | 3091 | GHDSPHKSGRT | 3132 | GHNSPHKSG | 3174 | GHDSPHKSGI | 3216 |
| QQT | | QET | | QNQET | | SIQT | |
| GHDSPHKSGQR | 3092 | GHDSPHKSVQT | 3133 | GHDSPHKSA | 3175 | VHDSPHKSG | 3217 |
| QHT | | HQT | | QNQQI | | QNQQT | |
| GHDSPHKSAQS | 3093 | GHDSPHKSGQN | 3134 | NHDSPHKSG | 3176 | GHDSPHKSV | 3218 |
| QQT | | QPA | | QNQQT | | QNLQT | |
| GHESPHKSGQN | 3094 | KHDSPHKSGQN | 3135 | GHDSPHKSG | 3177 | GHDSPHKMA | 3219 |
| QQS | | QQT | | QSQPT | | HNQQT | |
| GHDSPHKSLQN | 3095 | GHDSPHKSGQT | 3136 | GHESPHKSG | 3178 | GHDSPHKHG | 3220 |
| QQT | | QQT | | QNEQT | | QNQQN | |
| GHDSPHKSGRN | 3096 | GPDSPHKIGQNQ | 3137 | GHDSPHKSA | 3179 | GHDSPHKSV | 3221 |
| QET | | QS | | QNQQT | | QSQQS | |
| GHDSPHKSGQT | 3097 | GHDSPHKSVQN | 3138 | GHDSPHKSG | 3180 | GHDSPHKSG | 3222 |
| QLT | | QQT | | QNQLT | | QTVCT | |
| GHDSPHKSEKN | 3098 | GHDSPHKSRQD | 3139 | GHDSPHKSA | 3181 | GQDSPHKSG | 3223 |
| QQT | | QHT | | QNLLT | | QYQQI | |
| GRDSPHKSGQD | 3099 | GPDSPHKSGQK | 3140 | THDSPHKSG | 3182 | GHDSPHKSG | 3224 |
| QQT | | QQT | | QNQQT | | QQIMT | |
| GHDSPHKTGHN | 3100 | GHDSPHKSRQS | 3141 | GNGSPHKRG | 3183 | GHDSPHKSR | 3225 |
| QQT | | QHT | | QNQQT | | QNEQS | |
| AHDSPHKSGQN | 3101 | GHDSPHKSVQN | 3142 | GHDSPHKSG | 3184 | GHDSPHKSG | 3226 |
| QLT | | QLT | | QSLQT | | LNHQT | |
| GYDSPHKSGQT | 3102 | AHDSPHKSGQN | 3143 | GHDSPHKSA | 3185 | GYDSPHKSG | 3227 |
| QQT | | QQT | | QNHQT | | QNQQT | |
| GHESPHKSGQT | 3103 | GQDSPHKSGQN | 3144 | GHDSPHKSG | 3186 | GHDSPHKSG | 3228 |
| QQI | | QQS | | RNRQT | | QNLQT | |
| GHDSPHKSGQS | 3104 | GHDSPHKSGRN | 3145 | EHDSPHKSG | 3187 | GHDSPHKSR | 3229 |
| KQA | | QQI | | QNQQT | | QDQQT | |
| GHESPHKSGQN | 3105 | GYDSPHKSGQY | 3146 | GHDSPHKYG | 3188 | GDDSPHKSG | 3230 |
| QQT | | QHT | | QNEQT | | QKQLT | |
| GHDSPHKTGQN | 3106 | GHDSPHKSRQN | 3147 | RHDSPHKSG | 3189 | GDDSPHKSG | 3231 |
| QPP | | QQT | | HNQQT | | HNQQT | |
| GHDSPHKSGQSP | 3107 | GHDSPHKSWVR | 3148 | GHDSPHKSQ | 3190 | GHDSPHKSG | 3232 |
| QT | | QQT | | QNQQT | | QMIHT | |
| GNDSPHKSVQN | 3108 | GHESPHKSGQN | 3149 | GHDSPHKSG | 3191 | GHDSPHKSG | 3233 |
| QQT | | QHS | | QNQQI | | RNHQS | |
| GHDSPHKSAQN | 3109 | GHDSPHKIGHN | 3150 | QHDSPHKSG | 3192 | GHDSPHKSV | 3234 |
| YQT | | QQT | | QNQQT | | QNRQT | |
| AHDSPHKIGQN | 3110 | WHDSPHKSGQN | 3151 | RHDSPHKIVQ | 3193 | GHDSPHKSG | 3235 |
| HQT | | QQT | | NQQT | | QKMNT | |
| GHESPHKSAQN | 3111 | RHDSPHKSGQN | 3152 | YHDSPHKSG | 3194 | GHDSPHKSG | 3236 |
| RQT | | QQT | | QNQQT | | QSQQN | |
| GHDSPHKSGQN | 3112 | GHDSPHKSNAW | 3153 | GHDSPHKSV | 3195 | GHDSPHKSG | 3237 |
| QQG | | QQT | | QSKQT | | QYQHA | |
| GQDSPHKIGQN | 3113 | GHDSPHKSGQS | 3154 | GNDSPHKIG | 3196 | GHDSPHKSG | 3117 |
| QQT | | VPT | | HNQQT | | QNQWT | |
| GHDSPHKSGQN | 3114 | GHESPHKSGQNI | 3155 | HHDSPHKSG | 3197 | GHDSPHKRG | 3158 |
| HLT | | QP | | QNQQT | | PDQQS | |
| GHDSPHKSGQY | 3115 | GHDSPHKSVQN | 3156 | GHDSPHKSG | 3198 | GHDSPHKSG | 3200 |
| QHT | | HLN | | QTQQI | | RDQKT | |
| GNDSPHKSVQN | 3116 | GHDSPHKIGLDQ | 3157 | GQDSPHKSG | 3199 | GHDSPHKSV | 3201 |
| HQT | | QT | | QNPLT | | HNQQN | |
| GHDSPHKSVQN | 3118 | GHDSPHKSGES | 3159 | KTISKRGSPH | 4098 | KGLGGSGSP | 4099 |
| QHT | | QQT | | SKAQNQQT | | HSKAQNQQT | |
| KTINGHDSPHSK | 4100 | KTINGSGSPHSK | 4196 | KEIYGSGSPH | 4292 | KTINGSGSPH | 4388 |
| AQNLQT | | TCIQQT | | SKAQNQQT | | KRGQKQQT | |
| KTINGHDSPHSK | 4101 | KTINGSGSPHSK | 4197 | KELSGSGSPH | 4293 | KTINGSGSPH | 4389 |
| AQNQQI | | WLTQQT | | SKAQNQQT | | KRGQNQET | |
| KTINGSGSPHFT | 4102 | KTINGSGSPHSK | 4198 | KETIGSGSPH | 4294 | KTINGSGSPH | 4390 |
| RQNQQT | | WVVQQT | | SKAQNQQT | | KRGQNQLT | |
| KTINGSGSPHSL | 4103 | KTINGSGSPHSK | 4199 | KEVLGSGSP | 4295 | KTINGSGSPH | 4391 |
| PWNQQT | | YRLQQT | | HSKAQNQQT | | KRGRNQQT | |
| KTINGHDSPHSK | 4104 | KTINGSGSPHSK | 4200 | KFALGHDSP | 4296 | KTINGSGSPH | 4392 |
| AQNHQT | | YSKQQT | | HKSGQKQQT | | KSGGNQQT | |
| KTMNGHDSPHS | 4105 | KTINGSGSPHSK | 4201 | KIINGHDSPH | 4297 | KTINGSGSPH | 4393 |
| KAQNQQT | | YSRQQT | | KSGQNLVL | | KSGHNQET | |
| KPYKGSGSPHSK | 4106 | KTINGSGSPHSL | 4202 | KIINGHDSPH | 4298 | KTINGSGSPH | 4394 |
| AQNQQT | | KRNQQT | | KSGQRNYT | | KSGHNQLT | |
| KRLWGSGSPHS | 4107 | KTINGSGSPHSL | 4203 | KIINGHDSPH | 4299 | KTINGSGSPH | 4395 |
| KAQNQQT | | WFNQQT | | SKAQNQQT | | KSGHNQQN | |
| KRMRGSGSPHS | 4108 | KTINGSGSPHSL | 4204 | KLNPGHDSP | 4300 | KTINGSGSPH | 4396 |
| KAQNQQT | | WPNQQT | | HKSGQTQQT | | KSGLNQLT | |
| KRTYGSGSPHSK | 4109 | KTINGSGSPHSL | 4205 | KLNRGHDSP | 4301 | KTINGSGSPH | 4397 |
| AQNQQT | | WTNQQT | | HKSGQNQQS | | KSGPNQQT | |
| KTINCLRSPHSK | 4110 | KTINGSGSPHSM | 4206 | KLSSGHDSPH | 4302 | KTINGSGSPH | 4398 |
| AQNQQT | | RRNQQT | | KSGQNQQN | | KSGQGQQT | |
| KTINFSRSPHSK | 4111 | KTINGSGSPHSP | 4207 | KNINGHDSP | 4303 | KTINGSGSPH | 4399 |
| AQNQQT | | CLNQQT | | HSKAQNQQT | | KSGQHLQT | |
| KTINGLRSPHFK | 4112 | KTINGSGSPHSQ | 4208 | KNNDGSGSP | 4304 | KTINGSGSPH | 4400 |
| AQNQQT | | WQNQQT | | HSKAQNQQT | | KSGQHQQT | |
| KTINGNRSPHNK | 4113 | KTINGSGSPHSR | 4209 | KNVMGSGSP | 4305 | KTINGSGSPH | 4401 |
| AQNQQT | | CANQQT | | HSKAQNQQT | | KSGQKHQT | |
| KTINGPRSPHYK | 4114 | KTINGSGSPHSRI | 4210 | KPINGHDSPH | 4306 | KTINGSGSPH | 4402 |
| AQNQQT | | RNQQT | | KSGQNKLS | | KSGQKQQS | |
| KTINGQASPHW | 4115 | KTINGSGSPHSR | 4211 | KPINGHDSPH | 4307 | KTINGSGSPH | 4403 |
| KAQNQQT | | KSNQQT | | KSGQNLSS | | KSGQNEQT | |
| KTINGRCSPHSK | 4116 | KTINGSGSPHSR | 4212 | KPINGHDSPH | 4308 | KTINGSGSPH | 4404 |
| AQNQQT | | LWNQQT | | SKAQNQQT | | KSGQNHQT | |
| KTINGRHSPHSK | 4117 | KTINGSGSPHSR | 4213 | KRINGHDSPH | 4309 | KTINGSGSPH | 4405 |
| AQNQQT | | RFNQQT | | SKAQNQQT | | KSGQNKQT | |
| KTINGRKSPHRK | 4118 | KTINGSGSPHSR | 4214 | KSCSGHDSP | 4310 | KTINGSGSPH | 4406 |
| AQNQQT | | RPNQQT | | HKSGQNQQS | | KSGQNKTS | |
| KTINGRLSPHW | 4119 | KTINGSGSPHSR | 4215 | KSINGHDSPH | 4311 | KTINGSGSPH | 4407 |
| KAQNQQT | | SCNQQT | | KSGQNLAS | | KSGQNQEA | |
| KTINGRLSPHYK | 4120 | KTINGSGSPHSR | 4216 | KSINGHDSPH | 4312 | KTINGSGSPH | 4408 |
| AQNQQT | | SKNQQT | | KSGQNLFL | | KSGQNQET | |
| KTINGRPSPHMK | 4121 | KTINGSGSPHSR | 4217 | KSINGHDSPH | 4313 | KTINGSGSPH | 4409 |
| AQNQQT | | TKNQQT | | KSGQNLLM | | KSGQNQKT | |
| KTINGRSSPHWK | 4122 | KTINGSGSPHSR | 4218 | KSINGHDSPH | 4314 | KTINGSGSPH | 4410 |
| AQNQQT | | WLNQQT | | KSGQNLLQ | | KSGQNQQI | |
| KTINGRWSPHSK | 4123 | KTINGSGSPHSS | 4219 | KSINGHDSPH | 4315 | KTINGSGSPH | 4411 |
| AQNQQT | | VCNQQT | | KSGQNSLG | | KSGQNQQR | |
| KTINGSGSPHAP | 4124 | KTINGSGSPHSS | 4220 | KSINGHDSPH | 4316 | KTINGSGSPH | 4412 |
| CQNQQT | | WRNQQT | | KSGQNTLQ | | KSGQNQRT | |
| KTINGSGSPHA | 4125 | KTINGSGSPHSV | 4221 | KSINGHDSPH | 4317 | KTINGSGSPH | 4413 |
| WAQNQQT | | CQNQQT | | KSSSNQQT | | KSGQNQYT | |
| KTINGSGSPHCM | 4126 | KTINGSGSPHSV | 4222 | KSINGHDSPH | 4318 | KTINGSGSPH | 4414 |
| RQNQQT | | LCNQQT | | KYKLNQQT | | KSGQRQQT | |
| KTINGSGSPHFC | 4127 | KTINGSGSPHSV | 4223 | KSINGSGSPH | 4319 | KTINGSGSPH | 4415 |
| SQNQQT | | RRNQQT | | KSGQKQQT | | KSGQSQQT | |
| KTINGSGSPHFL | 4128 | KTINGSGSPHSV | 4224 | KSINGSGSPH | 4320 | KTINGSGSPH | 4416 |
| FQNQQT | | SCNQQT | | KSGQNQQT | | KSGQYQRT | |
| KTINGSGSPHFW | 4129 | KTINGSGSPHSW | 4225 | KSINGSGSPH | 4321 | KTINGSGSPH | 4417 |
| AQNQQT | | ALNQQT | | SKAQGLST | | KSGRNQQA | |
| KTINGSGSPHLC | 4130 | KTINGSGSPHSW | 4226 | KSINGSGSPH | 4322 | KTINGSGSPH | 4418 |
| AQNQQT | | ITNQQT | | SKAQLLGT | | KSRHNQQT | |
| KTINGSGSPHLR | 4131 | KTINGSGSPHSW | 4227 | KSINGSGSPH | 4323 | KTINGSGSPH | 4419 |
| YQNQQT | | PMNQQT | | SKTSWQQT | | KSRQYQQT | |
| KTINGSGSPHLY | 4132 | KTINGSGSPHSW | 4228 | KSMNGHDSP | 4324 | KTINGSGSPH | 4420 |
| YQNQQT | | RSNQQT | | HSKAQNQQT | | RKAQAPGT | |
| KTINGSGSPHPL | 4133 | KTINGSGSPHSY | 4229 | KSTLGSGSPH | 4325 | KTINGSGSPH | 4421 |
| CQNQQT | | FLNQQT | | SKAQNQHT | | SKAAMKQT | |
| KTINGSGSPHRI | 4134 | KTINGSGSPHSY | 4230 | KSTLGSGSPH | 4326 | KTINGSGSPH | 4422 |
| RQNQQT | | TYNQQT | | SKAQNQQN | | SKAGRQQT | |
| KTINGSGSPHRL | 4135 | KTINGSGSPHSY | 4231 | KSTVGSGSPH | 4327 | KTINGSGSPH | 442 |
| FQNQQT | | WQNQQT | | SKAQTQQT | | SKAGRTQT | |
| KTINGSGSPHSC | 4136 | KTINGSGSPHTL | 4232 | KTCKESGSPH | 4328 | KTINGSGSPH | 4424 |
| GQNQQT | | CQNQQT | | SKAQNQQT | | SKAKSNQT | |
| KTINGSGSPHSC | 4137 | KTINGSGSPHWL | 4233 | KTCKGSGSP | 4329 | KTINGSGSPH | 4425 |
| LRNQQT | | RQNQQT | | HSKAQNQQT | | SKALKTQT | |
| KTINGSGSPHSC | 4138 | KTINGSGSPHWP | 4234 | KTCKSSGSPH | 4330 | KTINGSGSPH | 4426 |
| LSNQQT | | SQNQQT | | SKAQNQQT | | SKAPRTQT | |
| KTINGSGSPHSC | 4139 | KTINGSGSPHYL | 4235 | KTDMGSGSP | 4331 | KTINGSGSPH | 4427 |
| RLNQQT | | RQNQQT | | HSKAQNQQT | | SKAQAART | |
| KTINGSGSPHSC | 4140 | KTINGSGSPHYT | 4236 | KTDNGIGSPH | 4332 | KTINGSGSPH | 4428 |
| SLNQQT | | RQNQQT | | SKAQNQQT | | SKAQAILT | |
| KTINGSGSPHSK | 4141 | KTINGSLSPHLW | 4237 | KTEGGSGSP | 4333 | KTINGSGSPH | 4429 |
| ACTLQT | | AQNQQT | | HSKAQNQQT | | SKAQCRGT | |
| KTINGSGSPHSK | 4142 | KTINGSPSPHCQ | 4238 | KTEHHSGSP | 4334 | KTINGSGSPH | 4430 |
| AFRAQT | | AQNQQT | | HSKAQNQQT | | SKAQGLRT | |
| KTINGSGSPHSK | 4143 | KTINGSRSPHLC | 4239 | KTEKDSGSP | 4335 | KTINGSGSPH | 4431 |
| AIRKQT | | AQNQQT | | HSKAQNQQT | | SKAQKGVL | |
| KTINGSGSPHSK | 4144 | KTINGSRSPHWR | 4240 | KTELGHDSP | 4336 | KTINGSGSPH | 4432 |
| AQASRT | | AQNQQT | | HKRGQNQQT | | SKAQKSNT | |
| KTINGSGSPHSK | 4145 | KTINGSVSPHWL | 4241 | KTESVSGSPH | 4337 | KTINGSGSPH | 4433 |
| AQFELT | | AQNQQT | | SKAQNQQT | | SKAQNNKF | |
| KTINGSGSPHSK | 4146 | KTINGTFSPHRK | 4242 | KTETNSGSPH | 4338 | KTINGSGSPH | 4434 |
| AQIVIT | | AQNQQT | | SKAQNQQT | | SKAQNRRT | |
| KTINGSGSPHSK | 4147 | KTINGWTSPHR | 4243 | KTETYSGSPH | 4339 | KTINGSGSPH | 4435 |
| AQLART | | KAQNQQT | | SKAQNQQT | | SKAQPKQT | |
| KTINGSGSPHSK | 4148 | KTINRGISPHSK | 4244 | KTEWLSGSP | 4340 | KTINGSGSPH | 4436 |
| AQLQRT | | AQNQQT | | HSKAQNQQT | | SKAQRAPT | |
| KTINGSGSPHSK | 4149 | KTINTVRSPHSK | 4245 | KTENGSGSPH | 4341 | KTINGSGSPH | 4437 |
| AQNARR | | AQNQQT | | KSGQNQQT | | SKAQREHT | |
| KTINGSGSPHSK | 4150 | KTKLRSGSPHSK | 4246 | KTGLRHDSP | 4342 | KTINGSGSPH | 4438 |
| AQNCPR | | AQNQQT | | HKSGQKQQT | | SKAQRFGT | |
| KTINGSGSPHSK | 4151 | KTRLRSGSPHSK | 4247 | KTGLRHDSP | 4343 | KTINGSGSPH | 4439 |
| AQNMRR | | AQNQQT | | HKSGQNQQS | | SKAQRPCT | |
| KTINGSGSPHSK | 4152 | KWLLGSGSPHS | 4248 | KTGVTHDSP | 4344 | KTINGSGSPH | 4440 |
| AQNRRV | | KAQNQQT | | HKSGQKQQT | | SKAQRQAT | |
| KTINGSGSPHSK | 4153 | KWSQGSGSPHS | 4249 | KTIDGHESPH | 4345 | KTINGSGSPH | 4441 |
| AQPSRT | | KAQNQQT | | SKAQNQQT | | SKAQRQPT | |
| KTINGSGSPHSK | 4154 | KWYLGSGSPHS | 4250 | KTIEGHDSPH | 4346 | KTINGSGSPH | 4442 |
| AQQVKT | | KAQNQQT | | KSGQTQQT | | SKAQTKLT | |
| KTINGSGSPHSK | 4155 | KYHSGSGSPHS | 4251 | KTIHGHDSPH | 4347 | KTINGSGSPH | 4443 |
| AQQVRT | | KAQNQQT | | SKAQNQQT | | SKAQTTHT | |
| KTINGSGSPHSK | 4156 | KYLPGSGSPHSK | 4252 | KTIHGHESPH | 4348 | KTINGSGSPH | 4444 |
| AQRLKT | | AQNQQT | | SKAQNQQT | | SKAQVQRT | |
| KTINGSGSPHSK | 4157 | KAINGGGSPHSK | 4253 | KTIIGHDSPH | 4349 | KTINGSGSPH | 4445 |
| AQRRAT | | TQNQQT | | KSGQNRSS | | SKAQVVRT | |
| KTINGSGSPHSK | 4158 | KAINGHDSPHK | 4254 | KTIIGHDSPH | 4350 | KTINGSGSPH | 4446 |
| AQRRGT | | RSPNQQT | | KSGQRLGT | | SKAQWPNT | |
| KTINGSGSPHSK | 4159 | KAINGHDSPHKS | 4255 | KTIIGSGSPH | 4351 | KTINGSGSPH | 4447 |
| AQRRRT | | FSPQQT | | KSGQNQQT | | SKAQYPST | |
| KTINGSGSPHSK | 4160 | KAINGHDSPHKS | 4256 | KTIKGHDSPH | 4352 | KTINGSGSPH | 4448 |
| AQRTRT | | GENQQP | | KSGQNMLF | | SKARALQT | |
| KTINGSGSPHSK | 4161 | KAINGHDSPHKS | 4257 | KTILGSGSPH | 4353 | KTINGSGSPH | 4449 |
| AQRVHT | | GQLART | | SKAQNLQT | | SKARDQHT | |
| KTINGSGSPHSK | 4162 | KAINGHDSPHKS | 4258 | KTINGCSSPH | 4354 | KTINGSGSPH | 4450 |
| AQTYRT | | GQNAFL | | WKAQNQQT | | SKARFQQT | |
| KTINGSGSPHSK | 4163 | KAINGHDSPHKS | 4259 | KTINGGGSTH | 4355 | KTINGSGSPH | 4451 |
| AQVRKT | | GQNAYT | | SKAQNQQT | | SKARRTQT | |
| KTINGSGSPHSK | 4164 | KAINGHDSPHKS | 4260 | KTINGHDSPH | 4356 | KTINGSGSPH | 4452 |
| ARGRQT | | GQNFAS | | KAGQSQQT | | SKARSLQT | |
| KTINGSGSPHSK | 4165 | KAINGHDSPHKS | 4261 | KTINGHDSPH | 4357 | KTINGSGSPH | 4453 |
| ARLCQT | | GQNLAS | | KRGQNVPS | | SKARVIQT | |
| KTINGSGSPHSK | 4166 | KAINGHDSPHKS | 4262 | KTINGHDSPH | 4358 | KTINGSGSPH | 4454 |
| ARLKQT | | GQNLGS | | KRGRSYQT | | SKAWYLQT | |
| KTINGSGSPHSK | 4167 | KAINGHDSPHKS | 4263 | KTINGHDSPH | 4359 | KTINGSGSPH | 4455 |
| ARNSQT | | GQNLKF | | KTGQNPPT | | SKGGGQQT | |
| KTINGSGSPHSK | 4168 | KAINGHDSPHKS | 4264 | KTINGHDSPH | 4360 | KTINGSGSPH | 4456 |
| ARWVQT | | GQNLLK | | SKAENQQT | | SKGSRQQT | |
| KTINGSGSPHSK | 4169 | KAINGHDSPHKS | 4265 | KTINGHDSPH | 4361 | KTINGSGSPH | 4457 |
| AVRWQT | | GQNLSR | | SKALSLQT | | SKLQRQQT | |
| KTINGSGSPHSK | 4170 | KAINGHDSPHKS | 4266 | KTINGHDSPH | 4362 | KTINGSGSPH | 4458 |
| AYTRQT | | GQNLSS | | SKAQGQQT | | SKMLRQQT | |
| KTINGSGSPHSK | 4171 | KAINGHDSPHKS | 4267 | KTINGHDSPH | 4363 | KTINGSGSPH | 4459 |
| CQSQQT | | GQNSLG | | SKAQHQQT | | SKSSIKQT | |
| KTINGSGSPHSK | 4172 | KAINGHDSPHKS | 4268 | KTINGHDSPH | 4364 | KTINGSGSPH | 4460 |
| FLRQQT | | GQNTLQ | | SKAQIQQT | | SKVRFQQT | |
| KTINGSGSPHSK | 4173 | KAINGHDSPHKS | 4269 | KTINGHDSPH | 4365 | KTINGSGSPH | 4461 |
| FRFQQT | | GQNTSL | | SKAQKQQT | | SVVWNQQT | |
| KTINGSGSPHSK | 4174 | KAINGHDSPHKS | 4270 | KTINGHDSPH | 4366 | KTINGSTSPH | 4462 |
| FRLQQT | | GQRLGT | | SKAQNLSS | | KLAQNQQP | |
| KTINGSGSPHSK | 4175 | KAINGHDSPHKS | 4271 | KTINGHDSPH | 4367 | KTINRHDSPH | 4463 |
| FRRQQT | | GQRNYT | | SKAQNPQT | | KSGQRPST | |
| KTINGSGSPHSK | 4176 | KAINGHDSPHKS | 4272 | KTINGHDSPH | 4368 | KTINRIMSPH | 4464 |
| GMKQQT | | GQRPST | | SKAQNQET | | SKAQNQQT | |
| KTINGSGSPHSK | 4177 | KAINGHDSPHKS | 4273 | KTINGHDSPH | 4369 | KTINTARSPH | 4465 |
| KLRQQT | | GQRPVT | | SKAQNQHT | | SKAQNQQT | |
| KTINGSGSPHSK | 4178 | KAINGHDSPHKS | 4274 | KTINGHDSPH | 4370 | KTISGHDSPH | 4466 |
| KRPQQT | | GQVPST | | SKAQNQLT | | SKAQNQQT | |
| KTINGSGSPHSK | 4179 | KAINGHDSPHKS | 4275 | KTINGHDSPH | 4371 | KTISGSGSPH | 4467 |
| KSRQQT | | LSNQQT | | SKAQNQPT | | KSGQNQQT | |
| KTINGSGSPHSK | 4180 | KAINGHDSPHKS | 4276 | KTINGHDSPH | 4372 | KTITGHDSPH | 4468 |
| LYRQQT | | VLSQQT | | SKAQNQQA | | KSGQRLGT | |
| KTINGSGSPHSK | 4181 | KAINGHDSPHKT | 4277 | KTINGHDSPH | 4373 | KTITGSGSPH | 4469 |
| LYWQQT | | LQNQQT | | SKAQNTGS | | KSGQNQQT | |
| KTINGSGSPHSK | 4182 | KAINGHNSPHSK | 4278 | KTINGHDSPH | 4374 | KTIYGHDSP | 4470 |
| PRMQQT | | AQNQQT | | SKAQSQQT | | HKSGQRLGT | |
| KTINGSGSPHSK | 4183 | KAINGLDSPHSK | 4279 | KTINGHDSPH | 4375 | KTLNGHDSP | 4471 |
| RFPQQT | | AQNQQT | | SKAQTQQT | | HKSGQNLFL | |
| KTINGSGSPHSK | 4184 | KAINGSGSPHKS | 4280 | KTINGHDSPH | 4376 | KTLNGHDSP | 4472 |
| RFRQQT | | GQNQQT | | SKAQYQQT | | HKSGQNLSS | |
| KTINGSGSPHSK | 4185 | KAINGSGSPHSK | 4281 | KTINGHDSPH | 4377 | KTLSFHDSPH | 4473 |
| RPYQQT | | AQGQQT | | SKARNQQT | | KSGQNQQS | |
| KTINGSGSPHSK | 4186 | KAINGSGSPHSK | 4282 | KTINGHDSPH | 4378 | KTSNGSGSP | 4474 |
| RRMQQT | | AQLSGT | | SKLPGQQT | | HSKAQNTMT | |
| KTINGSGSPHSK | 4187 | KAINGSGSPHSK | 4283 | KTINGHDSPH | 4379 | KTTNGHDSP | 4475 |
| RSKQQT | | AQNGSL | | SKSPNQQT | | HSKAQNQQT | |
| KTINGSGSPHSK | 4188 | KAINGSGSPHSK | 4284 | KTINGHESPH | 4380 | KTVNGGGSP | 4476 |
| RSRQQT | | AQNSLL | | KSGQNAFL | | HSKAQNQQT | |
| KTINGSGSPHSK | 4189 | KAINGSGSPHSK | 4285 | KTINGIGSPH | 4381 | KTVNGHDSP | 4477 |
| RTMQQT | | AVGLQT | | SKAPNEQT | | HKSGQNVSL | |
| KTINGSGSPHSK | 4190 | KAINGSGSPHSK | 4286 | KTINGQDSPH | 4382 | KTVNGHDSP | 4478 |
| RTRQQT | | SLLQQT | | KSGQNLHM | | HKSGQRPST | |
| KTINGSGSPHSK | 4191 | KAINGSGSPHSK | 4287 | KTINGRGSPH | 4383 | KTVNGHDSP | 4479 |
| RVRQQT | | SLPQQT | | SKAQIGMT | | HKSGQTQQA | |
| KTINGSGSPHSK | 4192 | KAINGSGSPHSK | 4288 | KTINGRGSPH | 4384 | KTVNGHESP | 4480 |
| RYIQQT | | STFQQT | | SKAQNQVL | | HSKAQNQQT | |
| KTINGSGSPHSK | 4193 | KAITGHDSPHSK | 4289 | KTINGRGSPH | 4385 | KTVNGSGSP | 4481 |
| RYNQQT | | AQNQQT | | SKAQSPTT | | HSKAQGLST | |
| KTINGSGSPHSK | 4194 | KDVMGSGSPHS | 4290 | KTINGRGSPH | 4386 | KTVNGSGSP | 4482 |
| RYPQQT | | KAQNQQT | | SKATSFQT | | HSKAQNVTS | |
| KTINGSGSPHSK | 4195 | KEIVGSGSPHSK | 4291 | KTINGSGSPH | 4387 | KTVPASGSP | 4483 |
| RYSQQT | | AQNQQT | | FVVQNQQT | | HSKAQNQQT | |
| NTINGSGSPHSK | 4484 | TTINGGGSPHSK | 4485 | | | | |
| AHNQQT | | AQNQQT | | | | | |

TABLE 2A
Exemplary Peptide Sequences

| SEQ | Amino | SEQ | |
|---|---|---|---|
| ID | Acid | ID | |
| NO: | Sequence | NO: | Nucleotide Sequence |
| 941 | SPHSKA | 942 | AGCCCACACAGCAAAGCA |
| 943 | HDSPHKSG | 944 | CACGACAGCCCACACAAAAGCGGA |
| 2 | HDSPHK | 3 | CACGACAGCCCACACAAA |

TABLE 2B
Exemplary Peptide Sequences

| Amino | SEQ | Amino | SEQ | Amino | SEQ | Amino | SEQ |
|---|---|---|---|---|---|---|---|
| Acid | ID | Acid | ID | Acid | ID | Acid | ID |
| Sequence | NO: | Sequence | NO: | Sequence | NO: | Sequence | NO: |
| SPHSKA | 945 | SPHKKN | 954 | SPHKTS | 963 | SPHTRG | 972 |
| SPHKSG | 946 | SPHVRM | 955 | SPHKTT | 964 | SPHVRG | 973 |
| SPHARM | 947 | SPHRKA | 956 | SPHKTY | 965 | SPHKRG | 974 |
| SPHVKS | 948 | SPHKFG | 957 | SPHKYG | 966 | SPHGAR | 975 |
| SPHASR | 949 | SPHKIG | 958 | SPHSKD | 967 | SPHRSG | 976 |
| SPHVKI | 950 | SPHKLG | 959 | SPHSKP | 968 | SPHKSA | 977 |
| SPHKSR | 951 | SPHSKL | 960 | SPHSRA | 969 | SPHSKR | 978 |
| SPHSLR | 952 | SPHSRG | 961 | SPHSSR | 970 | SPHFLR | 979 |
| SPHSKW | 953 | SPHSKS | 962 | SPHWKA | 971 | SPHVRR | 980 |
| STHASR | 985 | SQHKSG | 986 | HDSPHK | 2 | HDSPHSKA | 4486 |

In some embodiments, an AAV capsid variant described herein comprises an amino acid sequence having the formula [N1]-[N2]-[N3], wherein [N2] comprises the amino acid sequence of SPH and [N3] comprises X4, X5, and X6, wherein at least one of X4, X5, or X6 is a basic amino acid, e.g., a K or R. In some embodiments, position X4 of [N2] is K. In some embodiments, position X5 of [N2] is K.

In some embodiments, [N1] comprises X1, X2, and X3, wherein at least one of X1, X2, or X3 is G. In some embodiments, position X1 of [N1] is: G, V, R, D, E, M, T, I, S, A, N, L, K, H, P, W, or C. In some embodiments, position X2 of [N1] is: S, V, L, N, D, H, R, P, G, T, I, A, E, Y, M, or Q. In some embodiments, position X3 of [N1] is: G, C, L, D, E, Y, H, V, A, N, P, or S. In some embodiments, [N1] comprises GS, SG, GH, HD, GQ, QD, VS, CS, GR, RG, QS, SH, MS, RN, TS, IS, GP, ES, SS, GN, AS, NS, LS, GG, KS, GT, PS, RS, GI, WS, DS, ID, GL, DA, DG, ME, EN, KN, KE, AI, NG, PG, TG, SV, IG, LG, AG, EG, SA, YD, HE, HG, RD, ND, PD, MG, QV, DD, HN, HP, GY, GM, GD, or HS. In some embodiments, [N1] comprises GS, SG, GH, or HD. In some embodiments [N1] is or comprises GSG, GHD, GQD, VSG, CSG, CSH, GQS, GRG, GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS. In some embodiments, [N1] is or comprises GSG. In some embodiments, [N1] is or comprises GHD. In some embodiments, [N1]-[N2] comprises SGSPH (SEQ ID NO: 6668), HDSPH (SEQ ID NO: 6619), QDSPH (SEQ ID NO: 6669), RGSPH (SEQ ID NO: 6670), SHSPH (SEQ ID NO: 6671), QSSPH (SEQ ID NO: 6672), DDSPH (SEQ ID NO: 6673), HESPH (SEQ ID NO: 6674), NYSPH (SEQ ID NO: 6675), VGSPH (SEQ ID NO: 6676), SCSPH (SEQ ID NO: 6677), LLSPH (SEQ ID NO: 6678), NGSPH (SEQ ID NO: 6679), PGSPH (SEQ ID NO: 6680), GGSPH (SEQ ID NO: 6681), TGSPH (SEQ ID NO: 6682), SVSPH (SEQ ID NO: 6683), IGSPH (SEQ ID NO: 6684), DGSPH (SEQ ID NO: 6685), LGSPH (SEQ ID NO: 6686), AGSPH (SEQ ID NO: 6687), EGSPH (SEQ ID NO: 6688), SASPH (SEQ ID NO: 6689), YDSPH (SEQ ID NO: 6690), HGSPH (SEQ ID NO: 6691), RDSPH (SEQ ID NO: 6692), NDSPH (SEQ ID NO: 6693), PDSPH (SEQ ID NO: 6694), MGSPH (SEQ ID NO: 6695), QVSPH (SEQ ID NO: 6696), HNSPH (SEQ ID NO: 6697), HPSPH (SEQ ID NO: 6698), or HSSPH (SEQ ID NO: 6699); an amino acid sequence comprising any portion of any of the aforesaid amino acid sequences (e.g., any 2, 3, or 4 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2] is or comprises GSGSPH (SEQ ID NO: 6700), GHDSPH (SEQ ID NO: 6701), GQDSPH (SEQ ID NO: 6702), VSGSPH (SEQ ID NO: 6703), CSGSPH (SEQ ID NO: 6704), GRGSPH (SEQ ID NO: 6705), CSHSPH (SEQ ID NO: 6706), GQSSPH (SEQ ID NO: 6707), GSHSPH (SEQ ID NO: 6708), GDDSPH (SEQ ID NO: 6709), GHESPH (SEQ ID NO: 6710), GNYSPH (SEQ ID NO: 6711), RVGSPH (SEQ ID NO: 6712), GSCSPH (SEQ ID NO: 6713), GLLSPH (SEQ ID NO: 6714), MSGSPH (SEQ ID NO: 6715), RNGSPH (SEQ ID NO: 6716), TSGSPH (SEQ ID NO: 6717), ISGSPH (SEQ ID NO: 6718), GPGSPH (SEQ ID NO: 6719), ESGSPH (SEQ ID NO: 6720), SSGSPH (SEQ ID NO: 6721), GNGSPH (SEQ ID NO: 6722), ASGSPH (SEQ ID NO: 6723), NSGSPH (SEQ ID NO: 6724), LSGSPH (SEQ ID NO: 6725), GGGSPH (SEQ ID NO: 6726), KSGSPH (SEQ ID NO: 6727), HSGSPH (SEQ ID NO: 6728), GTGSPH (SEQ ID NO: 6729), PSGSPH (SEQ ID NO: 6730), GSVSPH (SEQ ID NO: 6731), RSGSPH (SEQ ID NO: 6732), GIGSPH (SEQ ID NO: 6733), WSGSPH (SEQ ID NO: 6734), DSGSPH (SEQ ID NO: 6735), IDGSPH (SEQ ID NO: 6736), GLGSPH (SEQ ID NO: 6737), DAGSPH (SEQ ID NO: 6738), DGGSPH (SEQ ID NO: 6739), MEGSPH (SEQ ID NO: 6740), ENGSPH (SEQ ID NO: 6741), GSASPH (SEQ ID NO: 6742), KNGSPH (SEQ ID NO: 6743), KEGSPH (SEQ ID NO: 6744), AIGSPH (SEQ ID NO: 6745), GYDSPH (SEQ ID NO: 6746), GHGSPH (SEQ ID NO: 6747), GRDSPH (SEQ ID NO: 6748), GNDSPH (SEQ ID NO: 6749), GPDSPH (SEQ ID NO: 6750), GMGSPH (SEQ ID NO: 6751), GQVSPH (SEQ ID NO: 6752), GHNSPH (SEQ ID NO: 6753), GHPSPH (SEQ ID NO: 6754), or GHSSPH (SEQ ID NO: 6755); an amino acid sequence comprising any portion of any of the aforesaid amino acid sequences (e.g., any 2, 3, 4, or 5 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2] is or comprises GSGSPH (SEQ ID NO: 6700). In some embodiments, [N1]-[N2] is or comprises GHDSPH (SEQ ID NO: 6701).

In some embodiments, X4, X5, or both of [N3] are K. In some embodiments, X4, X5, or X6 of [N3] is R. In some embodiments, position X4 of [N3] is: A, K, V, S, T, G, F, W, V, N, or R. In some embodiments, position X5 of [N3] is: S, K, T, F, I, L, Y, H, M, or R. In some embodiments, position X6 of [N3] is: G, R, A, M, I, N, T, Y, D, P, V, L, E, W, N, Q, K, or S. In some embodiments, [N3] comprises SK, KA, KS, AR, RM, VK, AS, SR, VK, KR, KK, KN, VR, RS, RK, KT, TS, KF, FG, KI, IG, KL, LG, TT, TY, KY, YG, KD, KP, TR, RG, VR, GA, SL, SS, FL, WK, SA, RA, LR, KW, RR, GK, TK, NK, AK, KV, KG, KH, KM, TG, SE, SV, SW, SN, HG, SQ, LW, MG, MA, or SG. In some embodiments, [N3] comprises SK, KA, KS, or SG. In some embodiments, [N3] is or comprises SKA, KSG, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG. In some embodiments, [N3] is or comprises SKA. In some embodiments, [N3] is or comprises KSG. In some embodiments, [N2]-[N3] comprises SPHSK (SEQ ID NO: 6617), SPHKS (SEQ ID NO: 6620), SPHAR (SEQ ID NO: 6621), SPHVK (SEQ ID NO: 6622), SPHAS (SEQ ID NO: 6623), SPHKK (SEQ ID NO: 6624), SPHVR (SEQ ID NO: 6625), SPHRK (SEQ ID NO: 6626), SPHKT (SEQ ID NO: 6627), SPHKF (SEQ ID NO: 6628), SPHKI (SEQ ID NO: 6629), SPHKL (SEQ ID NO: 6630), SPHKY (SEQ ID NO: 6631), SPHTR (SEQ ID NO: 6632), SPHKR (SEQ ID NO: 6633), SPHGA (SEQ ID NO: 6634), SPHSR (SEQ ID NO: 6635), SPHSL (SEQ ID NO: 6636), SPHSS (SEQ ID NO: 6637), SPHFL (SEQ ID NO: 6638), SPHWK (SEQ ID NO: 6639), SPHGK (SEQ ID NO: 6640), SPHTK (SEQ ID NO: 6641), SPHNK (SEQ ID NO: 6642), SPHAK (SEQ ID NO: 6643), SPHKH (SEQ ID NO: 6644), SPHKM (SEQ ID NO: 6645), or SPHRS (SEQ ID NO: 6646). In some embodiments [N2]-[N3] comprises SPHSK (SEQ ID NO: 6617) or SPHKS (SEQ ID NO: 6620). In some embodiments, [N2]-[N3] is or comprises SPHSKA (SEQ ID NO: 941), SPHKSG (SEQ ID NO: 946), SPHARM (SEQ ID NO: 947), SPHVKS (SEQ ID NO: 948), SPHASR (SEQ ID NO: 949), SPHVKI (SEQ ID NO: 950), SPHKKN (SEQ ID NO: 954), SPHVRM (SEQ ID NO: 955), SPHRKA (SEQ ID NO: 956), SPHKFG (SEQ ID NO: 957), SPHKIG (SEQ ID NO: 958), SPHKLG (SEQ ID NO: 959), SPHKTS (SEQ ID NO: 963), SPHKTT (SEQ ID NO: 964), SPHKTY (SEQ ID NO: 965), SPHKYG (SEQ ID NO: 966), SPHSKD (SEQ ID NO: 967), SPHSKP (SEQ ID NO: 968), SPHTRG (SEQ ID NO: 972), SPHVRG (SEQ ID NO: 973), SPHKRG (SEQ ID NO: 974), SPHGAR (SEQ ID NO: 975), SPHKSA (SEQ ID NO: 977), SPHKSR (SEQ ID NO: 951), SPHSKL (SEQ ID NO: 960), SPHSRA (SEQ ID NO: 969), SPHSKR (SEQ ID NO: 978), SPHSLR (SEQ ID NO: 952), SPHSRG (SEQ ID NO: 961), SPHSSR (SEQ ID NO: 970), SPHFLR (SEQ ID NO: 979), SPHSKW (SEQ ID NO: 953), SPHSKS SPHWKA (SEQ ID NO: 7586), SPHVRR (SEQ ID NO: 980), SPHSKT (SEQ ID NO: 6647), SPHSKG (SEQ ID NO: 6648), SPHGKA (SEQ ID NO: 6649), SPHNKA (SEQ ID NO: 6650), SPHSKN (SEQ ID NO: 6651), SPHAKA (SEQ ID NO: 6652), SPHSKV (SEQ ID NO: 6653), SPHKTG (SEQ ID NO: 6654), SPHTKA (SEQ ID NO: 6655), SPHKSL (SEQ ID NO: 6656), SPHKSE (SEQ ID NO: 6657), SPHKSV (SEQ ID NO: 6658), SPHKSW (SEQ ID NO: 6659), SPHKSN (SEQ ID NO: 6660), SPHKHG (SEQ ID NO: 6661), SPHKSQ (SEQ ID NO: 6662), SPHKSK (SEQ ID NO: 6663), SPHKLW (SEQ ID NO: 6664), SPHWKG (SEQ ID NO: 6665), SPHKMG (SEQ ID NO: 6666), SPHKMA (SEQ ID NO: 6667), or SPHRSG (SEQ ID NO: 976). In some embodiments, [N2]-[N3] is SPHSKA (SEQ ID NO: 941). In some embodiments, [N2]-[N3] is or comprises SPHKSG (SEQ ID NO: 946).

In some embodiments, [N1]-[N2]-[N3] comprises SGSPHSK (SEQ ID NO: 6756), HDSPHKS (SEQ ID NO: 6757), SGSPHAR (SEQ ID NO: 6758), SGSPHVK (SEQ ID NO: 6759), QDSPHKS (SEQ ID NO: 6760), SGSPHKK (SEQ ID NO: 6761), SGSPHVR (SEQ ID NO: 6762), SGSPHAS (SEQ ID NO: 6763), SGSPHRK (SEQ ID NO: 6764), SGSPHKT (SEQ ID NO: 6765), SHSPHKS (SEQ ID NO: 6766), QSSPHRS (SEQ ID NO: 6767), RGSPHAS (SEQ ID NO: 6768), RGSPHSK (SEQ ID NO: 6769), SGSPHKF (SEQ ID NO: 6770), SGSPHKI (SEQ ID NO: 6771), SGSPHKL (SEQ ID NO: 6772), SGSPHKY (SEQ ID NO: 6773), SGSPHTR (SEQ ID NO: 6774), SHSPHKR (SEQ ID NO: 6775), SGSPHGA (SEQ ID NO: 6776), HDSPHKR (SEQ ID NO: 6777), DDSPHKS (SEQ ID NO: 6778), HESPHKS (SEQ ID NO: 6779), NYSPHKI (SEQ ID NO: 6780), SGSPHSR (SEQ ID NO: 6781), SGSPHSL (SEQ ID NO: 6782), SGSPHSS (SEQ ID NO: 6783), VGSPHSK (SEQ ID NO: 6784), SCSPHRK (SEQ ID NO: 6785), SGSPHFL (SEQ ID NO: 6786), LLSPHWK (SEQ ID NO: 6787), NGSPHSK (SEQ ID NO: 6788), PGSPHSK (SEQ ID NO: 6789), GGSPHSK (SEQ ID NO: 6790), TGSPHSK (SEQ ID NO: 6791), SVSPHGK (SEQ ID NO: 6792), SGSPHTK (SEQ ID NO: 6793), IGSPHSK (SEQ ID NO: 6794), DGSPHSK (SEQ ID NO: 6795), SGSPHNK (SEQ ID NO: 6796), LGSPHSK (SEQ ID NO: 6797), AGSPHSK (SEQ ID NO: 6798), EGSPHSK (SEQ ID NO: 6799), SASPHSK (SEQ ID NO: 6800), SGSPHAK (SEQ ID NO: 6801), HDSPHKI (SEQ ID NO: 6802), YDSPHKS (SEQ ID NO: 6803), HDSPHKT (SEQ ID NO: 6804), RGSPHKR (SEQ ID NO: 6805), HGSPHSK (SEQ ID NO: 6806), RDSPHKS (SEQ ID NO: 6807), NDSPHKS (SEQ ID NO: 6808), QDSPHKI (SEQ ID NO: 6809), PDSPHKI (SEQ ID NO: 6810), PDSPHKS (SEQ ID NO: 6811), MGSPHSK (SEQ ID NO: 6812), HDSPHKH (SEQ ID NO: 6813), QVSPHKS (SEQ ID NO: 6814), HNSPHKS (SEQ ID NO: 6815), NGSPHKR (SEQ ID NO: 6816), HDSPHKY (SEQ ID NO: 6817), NDSPHKI (SEQ ID NO: 6818), HDSPHKL (SEQ ID NO: 6819), HPSPHWK (SEQ ID NO: 6820), HDSPHKM (SEQ ID NO: 6821), or HSSPHRS (SEQ ID NO: 6822). In some embodiments, [N1]-[N2]-[N3] is GSGSPHSKA (SEQ ID NO: 6613), GHDSPHKSG (SEQ ID NO: 6615), GSGSPHARM (SEQ ID NO: 6823), GSGSPHVKS (SEQ ID NO: 6824), GQDSPHKSG (SEQ ID NO: 6825), GSGSPHASR (SEQ ID NO: 6826), GSGSPHVKI (SEQ ID NO: 6827), GSGSPHKKN (SEQ ID NO: 6828), GSGSPHVRM (SEQ ID NO: 6829), VSGSPHSKA (SEQ ID NO: 6830), CSGSPHSKA (SEQ ID NO: 6831), GSGSPHRKA (SEQ ID NO: 6832), CSGSPHKTS (SEQ ID NO: 6833), CSHSPHKSG (SEQ ID NO: 6834), GQSSPHRSG (SEQ ID NO: 6835), GRGSPHASR (SEQ ID NO: 6836), GRGSPHSKA (SEQ ID NO: 6837), GSGSPHKFG (SEQ ID NO: 6838), GSGSPHKIG (SEQ ID NO: 6839), GSGSPHKLG (SEQ ID NO: 6840), GSGSPHKTS (SEQ ID NO: 6841), GSGSPHKTT (SEQ ID NO: 6842), GSGSPHKTY (SEQ ID NO: 6843), GSGSPHKYG (SEQ ID NO: 6844), GSGSPHSKD (SEQ ID NO: 6845), GSGSPHSKP (SEQ ID NO: 6846), GSGSPHTRG (SEQ ID NO: 6847), GSGSPHVRG (SEQ ID NO: 6848), GSHSPHKRG (SEQ ID NO: 6849), GSHSPHKSG (SEQ ID NO: 6850), VSGSPHASR (SEQ ID NO: 6851), VSGSPHGAR (SEQ ID NO: 6852), VSGSPHKFG (SEQ ID NO: 6853), GHDSPHKRG (SEQ ID NO: 6854), GDDSPHKSG (SEQ ID NO: 6855), GHESPHKSA (SEQ ID NO: 6856), GHDSPHKSA (SEQ ID NO: 6857), GNYSPHKIG (SEQ ID NO: 6858), GHDSPHKSR (SEQ ID NO: 6859), GSGSPHSKL (SEQ ID NO: 6860), GSGSPHSRA (SEQ ID NO: 6861), GSGSPHSKR (SEQ ID NO: 6862), GSGSPHSLR (SEQ ID NO: 6863), GSGSPHSRG (SEQ ID NO: 6864), GSGSPHSSR (SEQ ID NO: 6865), RVGSPHSKA (SEQ ID NO: 6866), GSCSPHRKA (SEQ ID NO: 6867), GSGSPHFLR (SEQ ID NO: 6868), GSGSPHSKW (SEQ ID NO: 6869), GSGSPHSKS (SEQ ID NO: 6870), GLLSPHWKA (SEQ ID NO: 6871), GSGSPHVRR (SEQ ID NO: 6872), GSGSPHSKV (SEQ ID NO: 6873), MSGSPHSKA (SEQ ID NO: 6874), RNGSPHSKA (SEQ ID NO: 6875), TSGSPHSKA (SEQ ID NO: 6876), ISGSPHSKA (SEQ ID NO: 6877), GPGSPHSKA (SEQ ID NO: 6878), GSGSPHSKT (SEQ ID NO: 6879), ESGSPHSKA (SEQ ID NO: 6880), SSGSPHSKA (SEQ ID NO: 6881), GNGSPHSKA (SEQ ID NO: 6882), ASGSPHSKA (SEQ ID NO: 6883), NSGSPHSKA (SEQ ID NO: 6884), LSGSPHSKA (SEQ ID NO: 6885), GGGSPHSKA (SEQ ID NO: 6886), KSGSPHSKA (SEQ ID NO: 6887), GGGSPHSKS (SEQ ID NO: 6888), GSGSPHSKG (SEQ ID NO: 6889), HSGSPHSKA (SEQ ID NO: 6890), GTGSPHSKA (SEQ ID NO: 6891), PSGSPHSKA (SEQ ID NO: 6892), GSVSPHGKA (SEQ ID NO: 6893), RSGSPHSKA (SEQ ID NO: 6894), GSGSPHTKA (SEQ ID NO: 6895), GIGSPHSKA (SEQ ID NO: 6896), WSGSPHSKA (SEQ ID NO: 6897), DSGSPHSKA (SEQ ID NO: 6898), IDGSPHSKA (SEQ ID NO: 6899), GSGSPHNKA (SEQ ID NO: 6900), GLGSPHSKS (SEQ ID NO: 6901), DAGSPHSKA (SEQ ID NO: 6902), DGGSPHSKA (SEQ ID NO: 6903), MEGSPHSKA (SEQ ID NO: 6904), ENGSPHSKA (SEQ ID NO: 6905), GSASPHSKA (SEQ ID NO: 6906), GNGSPHSKS (SEQ ID NO: 6907), KNGSPHSKA (SEQ ID NO: 6908), KEGSPHSKA (SEQ ID NO: 6909), AIGSPHSKA (SEQ ID NO: 6910), GSGSPHSKN (SEQ ID NO: 6911), GSGSPHAKA (SEQ ID NO: 6912), GHDSPHKIG (SEQ ID NO: 6913), GYDSPHKSG (SEQ ID NO: 6914), GHESPHKSG (SEQ ID NO: 6915), GHDSPHKTG (SEQ ID NO: 6916), GRGSPHKRG (SEQ ID NO: 6917), GQDSPHKSG (SEQ ID NO: 6825), GHDSPHKSL (SEQ ID NO: 6918), GHGSPHSKA (SEQ ID NO: 6919), GHDSPHKSE (SEQ ID NO: 6920), VSGSPHSKA (SEQ ID NO: 6830), GRDSPHKSG (SEQ ID NO: 6921), GNDSPHKSV (SEQ ID NO: 6922), GQDSPHKIG (SEQ ID NO: 6923), GHDSPHKSV (SEQ ID NO: 6924), GPDSPHKIG (SEQ ID NO: 6925), GPDSPHKSG (SEQ ID NO: 6926), GHDSPHKSW (SEQ ID NO: 6927), GHDSPHKSN (SEQ ID NO: 6928), GMGSPHSKT (SEQ ID NO: 6929), GHDSPHKHG (SEQ ID NO: 6930), GQVSPHKSG (SEQ ID NO: 6931), GDDSPHKSV (SEQ ID NO: 6932), GHNSPHKSG (SEQ ID NO: 6933), GNGSPHKRG (SEQ ID NO: 6934), GHDSPHKYG (SEQ ID NO: 6935), GHDSPHKSQ (SEQ ID NO: 6936), GNDSPHKIG (SEQ ID NO: 6937), GHDSPHKSK (SEQ ID NO: 6938), GHDSPHKLW (SEQ ID NO: 6939), GHPSPHWKG (SEQ ID NO: 6940), GHDSPHKMG (SEQ ID NO: 6941), GHDSPHKMA (SEQ ID NO: 6942), or GHSSPHRSG (SEQ ID NO: 6943); an amino acid sequence comprising any portion of any of the aforesaid amino acid sequences (e.g., any 2, 3, 4, 5, 6, 7, or 8 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2]-[N3] is or comprises GSGSPHSKA (SEQ ID NO: 6613). In some embodiments, [N1]-[N2]-[N3] is or comprises GHDSPHKSG (SEQ ID NO: 6615).

In some embodiments, the AAV capsid variant comprising an amino acid sequence having the formula [N1]-[N2]-[N3], further comprises [N4], wherein [N4] comprises X7 X8 X9 X10. In some embodiments, position X7 of [N4] is: W, Q, K, R, G, L, V, S, P, H, K, I, M, A, E, or F. In some embodiments, position X8 of [N4] is: N, Y, C, K, T, H, R, D, V, S, P, G, W, E, F, A, I, M, Q, or L. In some embodiments, position X9 of [N4] is: Q, G, K, H, R, T, L, D, A, P, I, F, V, M, W, Y, S, E, N, or Y. In some embodiments, position X10 of [N4] is: Q, H, L, R, W, K, A, P, E, M, I, S, G, N, Y, C, V, T, D, or V. In some embodiments [N4] comprises QNQQ (SEQ ID NO: 6945), WNQQ (SEQ ID NO: 6946), QYYV (SEQ ID NO: 6947), RRQQ (SEQ ID NO: 6948), GCGQ (SEQ ID NO: 6949), LRQQ (SEQ ID NO: 6950), RNQQ (SEQ ID NO: 6951), VNQQ (SEQ ID NO: 6952), FRLQ (SEQ ID NO: 6953), FNQQ (SEQ ID NO: 6954), LLQQ (SEQ ID NO: 6955), SNQQ (SEQ ID NO: 6956), RLQQ (SEQ ID NO: 6957), LNQQ (SEQ ID NO: 6958), QRKL (SEQ ID NO: 6959), LRRQ (SEQ ID NO: 6960), QRLR (SEQ ID NO: 6961), QRRL (SEQ ID NO: 6962), RRLQ (SEQ ID NO: 6963), RLRQ (SEQ ID NO: 6964), SKRQ (SEQ ID NO: 6965), QLYR (SEQ ID NO: 6966), QLTV (SEQ ID NO: 6967), QNKQ (SEQ ID NO: 6968), KNQQ (SEQ ID NO: 6969), QKQQ (SEQ ID NO: 6970), QTQQ (SEQ ID NO: 6971), QNHQ (SEQ ID NO: 6972), QHQQ (SEQ ID NO: 6973), QNQH (SEQ ID NO: 6974), QHRQ (SEQ ID NO: 6975), LTQQ (SEQ ID NO: 6976), QNQW (SEQ ID NO: 6977), QNTH (SEQ ID NO: 6978), RRRQ (SEQ ID NO: 6979), QYQQ (SEQ ID NO: 6980), QNDQ (SEQ ID NO: 6981), QNRH (SEQ ID NO: 6982), RDQQ (SEQ ID NO: 6983), PNLQ (SEQ ID NO: 6984), HVRQ (SEQ ID NO: 6985), PNQH (SEQ ID NO: 6986), HNQQ (SEQ ID NO: 6987), QSQQ (SEQ ID NO: 6988), QPAK (SEQ ID NO: 6989), QNLA (SEQ ID NO: 6990), QNQL (SEQ ID NO: 6991), QGQQ (SEQ ID NO: 6992), LNRQ (SEQ ID NO: 6993), QNPP (SEQ ID NO: 6994), QNLQ (SEQ ID NO: 6995), QDQE (SEQ ID NO: 6996), QDQQ (SEQ ID NO: 6997), HWQQ (SEQ ID NO: 6998), PNQQ (SEQ ID NO: 6999), PEQQ (SEQ ID NO: 7000), QRTM (SEQ ID NO: 7001), LHQH (SEQ ID NO: 7002), QHRI (SEQ ID NO: 7003), QYIH (SEQ ID NO: 7004), QKFE (SEQ ID NO: 7005), QFPS (SEQ ID NO: 7006), QNPL (SEQ ID NO: 7007), QAIK (SEQ ID NO: 7008), QNRQ (SEQ ID NO: 7009), QYQH (SEQ ID NO: 7010), QNPQ (SEQ ID NO: 7011), QHQL (SEQ ID NO: 7012), QSPP (SEQ ID NO: 7013), QAKL (SEQ ID NO: 7014), KSQQ (SEQ ID NO: 7015), QDRP (SEQ ID NO: 7016), QNLG (SEQ ID NO: 7017), QAFH (SEQ ID NO: 7018), QNAQ (SEQ ID NO: 7019), HNQL (SEQ ID NO: 7020), QKLN (SEQ ID NO: 7021), QNVQ (SEQ ID NO: 7022), QAQQ (SEQ ID NO: 7023), QTPP (SEQ ID NO: 7024), QPPA (SEQ ID NO: 7025), QERP (SEQ ID NO: 7026), QDLQ (SEQ ID NO: 7027), QAMH (SEQ ID NO: 7028), QHPS (SEQ ID NO: 7029), PGLQ (SEQ ID NO: 7030), QGIR (SEQ ID NO: 7031), QAPA (SEQ ID NO: 7032), QIPP (SEQ ID NO: 7033), QTQL (SEQ ID NO: 7034), QAPS (SEQ ID NO: 7035), QNTY (SEQ ID NO: 7036), QDKQ (SEQ ID NO: 7037), QNHL (SEQ ID NO: 7038), QIGM (SEQ ID NO: 7039), LNKQ (SEQ ID NO: 7040), PNQL (SEQ ID NO: 7041), QLQQ (SEQ ID NO: 7042), QRMS (SEQ ID NO: 7043), QGIL (SEQ ID NO: 7044), QDRQ (SEQ ID NO: 7045), RDWQ (SEQ ID NO: 7046), QERS (SEQ ID NO: 7047), QNYQ (SEQ ID NO: 7048), QRTC (SEQ ID NO: 7049), QIGH (SEQ ID NO: 7050), QGAI (SEQ ID NO: 7051), QVPP (SEQ ID NO: 7052), QVQQ (SEQ ID NO: 7053), LMRQ (SEQ ID NO: 7054), QYSV (SEQ ID NO: 7055), QAIT (SEQ ID NO: 7056), QKTL (SEQ ID NO: 7057), QLHH (SEQ ID NO: 7058), QNII (SEQ ID NO: 7059), QGHH (SEQ ID NO: 7060), QSKV (SEQ ID NO: 7061), QLPS (SEQ ID NO: 7062), IGKQ (SEQ ID NO: 7063), QAIH (SEQ ID NO: 7064), QHGL (SEQ ID NO: 7065), QFMC (SEQ ID NO: 7066), QNQM (SEQ ID NO: 7067), QHLQ (SEQ ID NO: 7068), QPAR (SEQ ID NO: 7069), QSLQ (SEQ ID NO: 7070), QSQL (SEQ ID NO: 7071), HSQQ (SEQ ID NO: 7072), QMPS (SEQ ID NO: 7073), QGSL (SEQ ID NO: 7074), QVPA (SEQ ID NO: 7075), HYQQ (SEQ ID NO: 7076), QVPS (SEQ ID NO: 7077), RGEQ (SEQ ID NO: 7078), PGQQ (SEQ ID NO: 7079), LEQQ (SEQ ID NO: 7080), QNQS (SEQ ID NO: 7081), QKVI (SEQ ID NO: 7082), QNND (SEQ ID NO: 7083), QSVH (SEQ ID NO: 7084), QPLG (SEQ ID NO: 7085), HNQE (SEQ ID NO: 7086), QIQQ (SEQ ID NO: 7087), QVRN (SEQ ID NO: 7088), PSNQ (SEQ ID NO: 7089), QVGH (SEQ ID NO: 7090), QRDI (SEQ ID NO: 7091), QMPN (SEQ ID NO: 7092), RGLQ (SEQ ID NO: 7093), PSLQ (SEQ ID NO: 7094), QRDQ (SEQ ID NO: 7095), QAKG (SEQ ID NO: 7096), QSAH (SEQ ID NO: 7097), QSTM (SEQ ID NO: 7098), QREM (SEQ ID NO: 7099), QYRA (SEQ ID NO: 7100), QRQQ (SEQ ID NO: 7101), QWQQ (SEQ ID NO: 7102), QRMN (SEQ ID NO: 7103), GDSQ (SEQ ID NO: 7104), QKIS (SEQ ID NO: 7105), PSMQ (SEQ ID NO: 7106), SPRQ (SEQ ID NO: 7107), MEQQ (SEQ ID NO: 7108), QYQN (SEQ ID NO: 7109), QIRQ (SEQ ID NO: 7110), QSVQ (SEQ ID NO: 7111), RSQQ (SEQ ID NO: 7112), QNKL (SEQ ID NO: 7113), QIQH (SEQ ID NO: 7114), PRQQ (SEQ ID NO: 7115), HTQQ (SEQ ID NO: 7116), QRQH (SEQ ID NO: 7117), RNQE (SEQ ID NO: 7118), QSKQ (SEQ ID NO: 7119), QNQP (SEQ ID NO: 7120), QSPQ (SEQ ID NO: 7121), QTRQ (SEQ ID NO: 7122), QNLH (SEQ ID NO: 7123), QNQE (SEQ ID NO: 7124), LNQP (SEQ ID NO: 7125), QNQD (SEQ ID NO: 7126), QNLL (SEQ ID NO: 7127), QLVI (SEQ ID NO: 7128), RTQE (SEQ ID NO: 7129), QTHQ (SEQ ID NO: 7130), QDQH (SEQ ID NO: 7131), QSQH (SEQ ID NO: 7132), VRQQ (SEQ ID NO: 7133), AWQQ (SEQ ID NO: 7134), QSVP (SEQ ID NO: 7135), QNIQ (SEQ ID NO: 7136), LDQQ (SEQ ID NO: 7137), PDQQ (SEQ ID NO: 7138), ESQQ (SEQ ID NO: 7139), QRQL (SEQ ID NO: 7140), QIIV (SEQ ID NO: 7141), QKQS (SEQ ID NO: 7142), QSHQ (SEQ ID NO: 7143), QFVV (SEQ ID NO: 7144), QSQP (SEQ ID NO: 7145), QNEQ (SEQ ID NO: 7146), INQQ (SEQ ID NO: 7147), RNRQ (SEQ ID NO: 7148), RDQK (SEQ ID NO: 7149), QWKR (SEQ ID NO: 7150), ENRQ (SEQ ID NO: 7151), QTQP (SEQ ID NO: 7152), QKQL (SEQ ID NO: 7153), RNQL (SEQ ID NO: 7154), ISIQ (SEQ ID NO: 7155), QTVC (SEQ ID NO: 7156), QQIM (SEQ ID NO: 7157), LNHQ (SEQ ID NO: 7158), QNQA (SEQ ID NO: 7159), QMIH (SEQ ID NO: 7160), RNHQ (SEQ ID NO: 7161), or QKMN (SEQ ID NO: 7162), or any dipeptide or tripeptide thereof. In some embodiments, [N1]-[N2]-[N3]-[N4] is or comprises: the amino acid sequence of any of SEQ ID NOs: 1800-2241; an amino acid sequence comprising any portion of any of the aforesaid amino acid sequences (e.g., any 2, 3, 4, 5, 6, 7, 8, 9, 10, 11, or 12 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N1]-[N2]-[N3]-[N4] is or comprises GSGSPHSKAQNQQ (SEQ ID NO: 1801). In some embodiments, [N1]-[N2]-[N3]-[N4] is or comprises GHDSPHKSGQNQQ (SEQ ID NO: 1800).

In some embodiments, the AAV capsid variant comprising an amino acid sequence having the formula [N1]-[N2]-[N3], further comprises [N0], wherein [N0] comprises XA XB and XC. In some embodiments, XA of [N0] is: T, S, Y, M, A, C, I, R, L, D, F, V, Q, N, H, E, or G. In some embodiments, XB of [N0] is: I, M, P, E, N, D, S, A, T, G, Q, F, V, L, C, H, R, W, or L. In some embodiments, XC of [N0] is: N, M, E, G, Y, W, T, I, Q, F, V, A, L, I, P, K, R, H, S, D, or S. In some embodiments, [N0] comprises TIN, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TII, TFP, TEK, EIN, TVN, TFN, SIN, TER, TSY, ELH, AIN, SVN, TDN, TFH, TVH, TEN, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAI, AEN, AET, ETA, NNL, or any dipeptide thereof. In some embodiments, [N0]-[N1]-[N2]-[N3]-[N4] is or comprises the amino acid sequence of any one of SEQ ID NOs: 2242-2886; an amino acid sequence comprising any portion of any of the aforesaid amino acid sequences (e.g., any 2, 3, 4, 5, 6, 7, 8, 9, 10, 11, 12, 13, 14, or 15 amino acids, e.g., consecutive amino acids) thereof; an amino acid sequence comprising one, two, or three but no more than four modifications, e.g., substitutions (e.g., conservative substitutions), insertions, or deletions, relative to any of the aforesaid amino acid sequences; or an amino acid sequence comprising one, two, or three but no more than four different amino acids, relative to any one of the aforesaid amino acid sequences. In some embodiments, [N0]-[N1]-[N2]-[N3]-[N4] is or comprises TINGSGSPHSKAQNQQ (SEQ ID NO: 2242). In some embodiments, [N0]-[N1]-[N2]-[N3]-[N4] is or comprises TINGHDSPHKSGQNQQ (SEQ ID NO: 2243).

In some embodiments, [N1]-[N2]-[N3] is present in loop IV of the AAV capsid variant. In some embodiments [N0] and [N4] are present in loop IV of the AAV capsid variant. In some embodiments, [N0]-[N1]-[N2]-[N3]-[N4] is present in loop IV of the AAV capsid variant.

In some embodiments, [N0] is present immediately subsequent to position 449, relative to a reference sequence numbered according to the amino acid sequence of SEQ ID NO: 138. In some embodiments, wherein [N0] is present immediately subsequent to position 449, relative to a reference sequence numbered according to the amino acid sequence of SEQ ID NO: 981 or 982. In some embodiments, [N0] replaces positions 450, 451, and 452 (e.g., amino acids T450, I451, and N452), relative to a reference sequence numbered according to SEQ ID NO: 138, 981, or 982. In some embodiments, [N0] is present immediately subsequent to position 449 and wherein [N0] replaces positions 450-452 (e.g., T450, I451, and N452), relative to a reference sequence numbered according to SEQ ID NO: 138, 981, or 982. In some embodiments, [N1] is present immediately subsequent to position 452, relative to a reference sequence numbered according to the amino acid sequence of SEQ ID NO: 138, 981 or 982. In some embodiments, wherein [N1] replaces positions 453-455 (e.g., G453, 5454, and G455), relative to a reference sequence numbered according to SEQ ID NO: 138, 981, or 982. In some embodiments, [N1] is present immediately subsequent to position 452 and wherein [N1] replaces positions 453-455 (e.g., G453, S454, and G455), relative to a reference sequence numbered according to SEQ ID NO: 138, 981, or 982. In some embodiments, [N2] is present immediately subsequent to position 455, relative to a reference sequence numbered according to the amino acid sequence of SEQ ID NO: 138, 981, or 982. In some embodiments, [N2]-[N3] is present immediately subsequent to position 455, relative to a reference sequence numbered according to the amino acid sequence of SEQ ID NO: 138, 981, or 982. In some embodiments [N1]-[N2]-[N3] is present immediately subsequent to position 452, numbered relative to SEQ ID NO: 138, 981, or 982. In some embodiments, [N1]-[N2]-[N3] replaces positions 453-455 (e.g., G453, S454, and G455), relative to a reference sequence numbered according to SEQ ID NO: 138, 981, or 982. In some embodiments, [N1] is present immediately subsequent to position 452 and wherein [N1]-[N2]-[N3] replaces positions 453-455 (e.g., G453, S454, and G455), relative to a reference sequence numbered according to SEQ ID NO: 138, 981, or 982. In some embodiments, [N4] is present immediately subsequent to position 455, relative to a reference sequence numbered according to the amino acid sequence of SEQ ID NO: 138. In some embodiments, [N4] replaces positions 456-459 (e.g., Q456, N457, Q458, and Q459), relative to a reference sequence numbered according to the amino acid sequence of SEQ ID NO: 138. In some embodiments, [N4] is present immediately subsequent to position 455, and [N4] replaces positions 456-459 (e.g., Q456, N457, Q458, and Q459), relative to a reference sequence numbered according to the amino acid sequence of SEQ ID NO: 138. In some embodiments, [N2]-[N3]-[N4] replaces positions 456-459 (e.g., Q456, N457, Q458, and Q459), relative to a reference sequence numbered according to the amino acid sequence of SEQ ID NO: 138. In some embodiments, [N2]-[N3]-[N4] is present immediately subsequent to position 455, and wherein [N2]-[N3]-[N4] replaces positions 456-459 (e.g., Q456, N457, Q458, and Q459), relative to a reference sequence numbered according to the amino acid sequence of SEQ ID NO: 138. In some embodiments, [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138. In some embodiments, [N1]-[N2]-[N3]-[N4] is present immediately subsequent to position 452, and wherein [N1]-[N2]-[N3]-[N4] replaces positions 453-459 (e.g., G453, S454, G455, Q456, N457, Q458, and Q459), relative to a reference sequence numbered according to the amino acid sequence of SEQ ID NO: 138. In some embodiments, [N0]-[N1]-[N2]-[N3]-[N4] replaces positions 450-459 (e.g., T450, I451, N452, G453, S454, G455, Q456, N457, Q458, and Q459), relative to a reference sequence numbered according to the amino acid sequence of SEQ ID NO: 138. In some embodiments, [N0]-[N1]-[N2]-[N3]-[N4] is present immediately subsequent to position 449, and wherein [N0]-[N1]-[N2]-[N3]-[N4] replaces positions 450-459 (e.g., T450, I451. N452, G453, S454, G455, Q456, N457, Q458, and Q459), relative to a reference sequence numbered according to the amino acid sequence of SEQ ID NO: 138.

In some embodiments, [N3] is present immediately subsequent to [N2].

In some embodiments, the AAV capsid variant comprises from N-terminus to C-terminus, [N2]-[N3]. In some embodiments, the AAV capsid variant comprises from N-terminus to C-terminus, [N1]-[N2]-[N3]. In some embodiments, the AAV capsid variant comprises from N-terminus to C-terminus, [N1]-[N2]-[N3]-[N4]. In some embodiments, the AAV capsid variant comprises from N-terminus to C-terminus, [N0]-[N1]-[N2]-[N3]. In some embodiments, the AAV capsid variant comprises from N-terminus to C-terminus, [N0]-[N1]-[N2]-[N3]-[N4].

In some embodiments, an AAV capsid variant described herein comprises an amino acid sequence comprising at least 3, 4, 5, 6, 7, 8, 9, 10, 11, 12, 13, 16, or 17 consecutive amino acids from any one of the sequences provided in Tables 1A, 2A, 2B, 15, or 16. In some embodiments, the AAV capsid variant comprises an amino acid sequence comprising at least 3, 4, or 5 consecutive amino acids from any one of SEQ ID NOs: 945-980 or 985-986. In some embodiments, the AAV capsid variant comprises an amino acid sequence comprising at least 3, 4, 5, 6, 7, 8, 9, 10, 11, 12, or 13 consecutive amino acids from any one of SEQ ID NOs: 200, 201, 941, 943, 204, 208, 404, or 903-909. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138, 981, or 982. In some embodiments, the amino acid sequence is present immediately subsequent to position 455, numbered according to SEQ ID NO: 982. In some embodiments, the amino acid sequence is present immediately subsequent to position 455, numbered according to SEQ ID NO: 138. In some embodiments, the amino acid sequence is present immediately subsequent to position 453, numbered according to SEQ ID NO: 981. In some embodiments, the amino acid sequence is present immediately subsequent to position 453, numbered according to SEQ ID NO: 138. In some embodiments, the amino acid sequence replaces 1, 2, 3, 4, 5, 6, 7, 8, 9, 10, 11, or all of positions 499 (e.g., K499), 450 (e.g., T450), 451 (e.g., I451), 452 (e.g., N452), 453 (e.g., G453), 454 (e.g., S454), 455 (e.g., G455), 456 (e.g., Q456), 457 (e.g., N457), 458 (e.g., Q458), 459 (e.g., Q459), and 460 (e.g., T460), numbered according to SEQ ID NO: 138. In some embodiments, the AAV capsid variant comprises one or more amino acid substitutions at positions 499 (e.g., K499), 450 (e.g., T450), 451 (e.g., I451), 452 (e.g., N452), 453 (e.g., G453), 454 (e.g., S454), 455 (e.g., G455), 456 (e.g., Q456), 457 (e.g., N457), 458 (e.g., Q458), 459 (e.g., Q459), and 460 (e.g., T460), numbered according to SEQ ID NO: 138.

In some embodiments, the 3 consecutive amino acids comprise SPH. In some embodiments, the 4 consecutive amino acids comprise SPHS (SEQ ID NO: 6616). In some embodiments, the 5 consecutive amino acids comprise SPHSK (SEQ ID NO: 6617). In some embodiments, the 6 consecutive amino acids comprise SPHSKA (SEQ ID NO: 941).

In some embodiments, 3 consecutive amino acids comprise HDS. In some embodiments, the 4 consecutive amino acids comprise HDSP (SEQ ID NO: 6618). In some embodiments, the 5 consecutive amino acids comprise HDSPH (SEQ ID NO: 6619). In some embodiments, the 6 consecutive amino acids comprise HDSPHK (SEQ ID NO: 2).

In some embodiments, an AAV capsid variant described herein comprises an amino acid sequence comprising at least one, two, or three but no more than four modifications, e.g., substitutions (e.g., conservative substitutions), insertions, or deletions, relative to the amino acid sequence of any one of the sequences provided in Tables 1A, 2A, 2B, 15, or 16. In some embodiments, the AAV capsid variant comprises an amino acid sequence comprising at least one, two, or three but no more than four different amino acids, relative to the amino acid sequence of any one of the sequences provided in Tables 1A, 2A, 2B, 15, or 16. In some embodiments, the AAV capsid variant comprises an amino acid sequence comprising at least one, two, or three but no more than four modifications, e.g., substitutions (e.g., conservative substitutions), insertions, or deletions, relative to the amino acid sequence of any one of SEQ ID NOs: 945-980 or 985-986. In some embodiments, the AAV capsid variant comprises an amino acid sequence comprising at least one, two, or three but no more than four different amino acids, relative to the amino acid sequence of any one of SEQ ID NOs: 945-980 or 985-986. In some embodiments, the AAV capsid variant comprises an amino acid sequence comprising at least one, two, or three but no more than four modifications, e.g., substitutions (e.g., conservative substitutions), insertions, or deletions, relative to the amino acid sequence of any one of SEQ ID NOs: 2, 200, 201, 941, 943, 204, 208, 404, or 903-909. In some embodiments, the AAV capsid variant comprises an amino acid sequence comprising at least one, two, or three but no more than four different amino acids, from the amino acid sequence of any one of SEQ ID NOs: 2, 200, 201, 941, 943, 204, 208, 404, or 903-909. In some embodiments, the amino acid sequence is present in loop IV. In some embodiments, the amino acid sequence is present immediately subsequent to position 448, 452, 453, 455, relative to a reference sequence numbered according to the amino acid sequence of SEQ ID NO: 138, 981, or 982. In some embodiments, the amino acid sequence is present immediately subsequent to position 455, numbered according to SEQ ID NO: 982. In some embodiments, the amino acid sequence is present immediately subsequent to position 455, numbered according to SEQ ID NO: 138. In some embodiments, the amino acid sequence is present immediately subsequent to position 453, numbered according to SEQ ID NO: 981. In some embodiments, the amino acid sequence is present immediately subsequent to position 453, numbered according to SEQ ID NO: 138. In some embodiments, the amino acid sequence replaces 1, 2, 3, 4, 5, 6, 7, 8, 9, 10, 11, or all of positions 499 (e.g., K499), 450 (e.g., T450), 451 (e.g., I451), 452 (e.g., N452), 453 (e.g., G453), 454 (e.g., S454), 455 (e.g., G455), 456 (e.g., Q456), 457 (e.g., N457), 458 (e.g., Q458), 459 (e.g., Q459), and 460 (e.g., T460), numbered according to SEQ ID NO: 138.

In some embodiments, the AAV capsid variant comprises an amino acid sequence comprising at least one, two, or three but no more than four modifications, e.g., substitutions (e.g., conservative substitutions), insertions, or deletions, relative to the amino acid sequence of SPHSKA (SEQ ID NO: 941). In some embodiments, the AAV capsid variant comprises an amino acid sequence comprising at least one, two, or three, but no more than four different amino acids from the amino acid sequence of SPHSKA (SEQ ID NO: 941).

In some embodiments, the AAV capsid variant comprises an amino acid sequence comprising at least one, two, or three but no more than four modifications, e.g., substitutions (e.g., conservative substitutions), insertions, or deletions, relative to the amino acid sequence of HDSPHK (SEQ ID NO: 2). In some embodiments, the AAV capsid variant comprises an amino acid sequence comprising at least one, two, or three, but no more than four different amino acids that relative to the amino acid sequence of HDSPHK (SEQ ID NO: 2).

In some embodiments, the AAV capsid variant, comprises the amino acid sequence of any of the sequences provided in Tables 1A, 2A, 2B, 15, or 16. In some embodiments, the peptide comprises the amino acid sequence of any of SEQ ID NOs: 945-980 or 985-986. In some embodiments, the AAV capsid variant comprises the amino acid sequence of any of SEQ ID NOs: 2, 200, 201, 941, 943, 204, 208, 404, or 903-909. In some embodiments, the AAV capsid variant comprises the amino acid sequence of SEQ ID NO: 2. In some embodiments, the AAV capsid variant comprises the amino acid sequence of SEQ ID NO: 941. In some embodiments, the AAV capsid variant comprises the amino acid sequence of SEQ ID NO: 943. In some embodiments, the AAV capsid variant comprises the amino acid sequence of SEQ ID NO: 3589. In some embodiments, the AAV capsid variant comprises the amino acid sequence of SEQ ID NO: 1754. In some embodiments, the AAV capsid variant comprises the amino acid sequence of SEQ ID NO: 3241. In some embodiments, the AAV capsid variant comprises the amino acid sequence of SEQ ID NO: 4100. In some embodiments, the AAV capsid variant comprises the amino acid sequence of SEQ ID NO: 4062. In some embodiments, the AAV capsid variant comprises the amino acid sequence of SEQ ID NO: 4486. In some embodiments, the amino acid sequence is present in loop IV. In some embodiments, the amino acid sequence is present immediately subsequent to position 448, relative to a reference sequence numbered according to the amino acid sequence of SEQ ID NO: 138. In some embodiments, the amino acid sequence replaces positions 449-460 (e.g., K449, T450, I451, N452, G453, 5454, G455, Q456, N457, Q458, Q459, and T460), numbered relative to SEQ ID NO: 138. In some embodiments, the amino acid sequence is present immediately subsequent to position 448 and replaces positions 449-460 (e.g., K449, T450, I451, N452, G453, S454, G455, Q456, N457, Q458, Q459, and T460), numbered relative to SEQ ID NO: 138. In some embodiments, the amino acid sequence is present immediately subsequent to position 449, relative to a reference sequence numbered according to the amino acid sequence of SEQ ID NO: 138. In some embodiments, the amino acid sequence replaces positions 450-460 (e.g., T450, I451, N452, G453, S454, G455, Q456, N457, Q458, Q459, and T460), numbered relative to SEQ ID NO: 138. In some embodiments, the amino acid sequence is present immediately subsequent to position 449, and replaces positions 450-460 (e.g., T450, I451, N452, G453, S454, G455, Q456, N457, Q458, Q459, and T460), numbered relative to SEQ ID NO: 138. In some embodiments, the amino acid sequence is present immediately subsequent to position 450, relative to a reference sequence numbered according to the amino acid sequence of SEQ ID NO: 138. In some embodiments, the amino acid sequence replaces positions 451-460 (e.g., I451, N452, G453, S454, G455, Q456, N457, Q458, Q459, and T460), numbered relative to SEQ ID NO: 138. In some embodiments, the amino acid sequence is present immediately subsequent to position 450 and replaces positions 451-460 (e.g., I451, N452, G453, S454, G455, Q456, N457, Q458, Q459, and T460), numbered relative to SEQ ID NO: 138. In some embodiments, the amino acid sequence is present immediately subsequent to position 451, relative to a reference sequence numbered according to the amino acid sequence of SEQ ID NO: 138. In some embodiments, the amino acid sequence replaces positions 452-460 (e.g., N452, G453, S454, G455, Q456, N457, Q458, Q459, and T460), numbered relative to SEQ ID NO: 138. In some embodiments, the amino acid sequence is present immediately subsequent to position 451 and replaces positions 452-460 (e.g., N452, G453, S454, G455, Q456, N457, Q458, Q459, and T460), numbered relative to SEQ ID NO: 138. In some embodiments, the amino acid sequence is present immediately subsequent to position 452, relative to a reference sequence numbered according to the amino acid sequence of SEQ ID NO: 138. In some embodiments, the amino acid sequence replaces positions 453-460 (e.g., G453, S454, G455, Q456, N457, Q458, Q459, and T460), numbered relative to SEQ ID NO: 138. In some embodiments, the amino acid sequence is present immediately subsequent to position 452, and replaces positions 453-460 (e.g., G453, S454, G455, Q456, N457, Q458, Q459, and T460), numbered relative to SEQ ID NO: 138. In some embodiments, the amino acid sequence is present immediately subsequent to position 453, relative to a reference sequence numbered according to the amino acid sequence of SEQ ID NO: 138. In some embodiments, the amino acid sequence replaces positions 454 and 455 (e.g., S454 and G455), numbered according to SEQ ID NO: 138. In some embodiments, the amino acid sequence is present immediately subsequent to position 453, and replaces positions 454 and 455 (e.g., S454 and G455), numbered according to SEQ ID NO: 138. In some embodiments, the amino acid sequence replaces positions 454-460 (e.g., S454, G455, Q456, N457, Q458, Q459, and T460), numbered relative to SEQ ID NO: 138. In some embodiments, the amino acid sequence is present immediately subsequent to position 453, and replaces positions 454-460 (e.g., 5454, G455, Q456, N457, Q458, Q459, and T460), numbered relative to SEQ ID NO: 138. In some embodiments, the amino acid sequence is present immediately subsequent to position 454, relative to a reference sequence numbered according to the amino acid sequence of SEQ ID NO: 138. In some embodiments, the amino acid sequence is present immediately subsequent to position 454, relative to a reference sequence numbered according to the amino acid sequence of SEQ ID NO: 981. In some embodiments, the amino acid sequence replaces positions 455-460 (e.g., positions G455, Q456, N457, Q458, Q459, and T460), numbered relative to SEQ ID NO: 138. In some embodiments, the amino acid sequence is present immediately subsequent to positions 454, and replaces positions 455-460 (e.g., positions G455, Q456, N457, Q458, Q459, and T460), numbered relative to SEQ ID NO: 138. In some embodiments, the amino acid sequence is present immediately subsequent to position 455, relative to a reference sequence numbered according to the amino acid sequence of SEQ ID NO: 138. In some embodiments, the amino acid sequence is present immediately subsequent to position 455, relative to a reference sequence numbered according to the amino acid sequence of SEQ ID NO: 982. In some embodiments, the amino acid sequence replaces positions 456-460 (e.g., Q456, N457, Q458, Q459, and T460), numbered relative to SEQ ID NO: 138. In some embodiments, the amino acid sequence is present immediately subsequent to position 455, and replaces positions 456-460 (e.g., Q456, N457, Q458, Q459, and T460), numbered relative to SEQ ID NO: 138.

In some embodiments, the AAV capsid variant (e.g., an AAV capsid variant described herein), comprises an amino acid sequence encoded by the nucleotide sequence of SEQ ID NO: 3 or 942, or a nucleotide sequence substantially identical (e.g., having at least 70%, 75%, 80%, 85%, 90%, 92%, 95%, 97%, 98%, or 99% sequence identity) thereto. In some embodiments, the AAV capsid variant described herein, comprises an amino acid sequence encoded by the nucleotide sequence of SEQ ID NO: 3 or 942, or a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequence of SEQ ID NO: 3 or 942. In some embodiments, the AAV capsid variant comprises an amino acid sequence encoded by a nucleotide sequence comprising at least one, two, three, four, five, six, or seven, but no more than ten different nucleotides relative to the nucleotide sequence of SEQ ID NO: 3 or 942.

In some embodiments, the nucleotide sequence encoding the AAV capsid variant (e.g., an AAV capsid variant described herein), comprises the nucleotide sequence of SEQ ID NO: 942, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 942. In some embodiments, the nucleotide sequence encoding an AAV capsid variant described herein comprises a nucleotide sequence comprising at least one, two, three, four, five, six, or seven, but no more than ten different nucleotides, relative to the nucleotide sequence of SEQ ID NO: 942.

In some embodiments, the nucleotide sequence encoding the AAV capsid variant (e.g., an AAV capsid variant described herein), comprises the nucleotide sequence of SEQ ID NO: 3, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, relative to the nucleotide sequences of SEQ ID NO: 3. In some embodiments, the nucleotide sequence encoding an AAV capsid variant described herein comprises a nucleotide sequence comprising at least one, two, three, four, five, six, or seven, but no more than ten different nucleotides relative to the nucleotide sequence of SEQ ID NO: 3.

In some embodiments, an AAV capsid variant described herein comprises the amino acid sequence of SPHSKA (SEQ ID NO: 941), wherein the amino acid sequence is present immediately subsequent to position 455, relative to a reference sequence numbered according to the amino acid sequence of SEQ ID NO: 138. In some embodiments, an AAV capsid variant described herein comprises the amino acid sequence of SPHSKA (SEQ ID NO: 941), wherein the amino acid sequence is present immediately subsequent to position 455, relative to a reference sequence numbered according to the amino acid sequence of SEQ ID NO: 981.

In some embodiments, an AAV capsid variant described herein comprises the amino acid sequence of HDSPHK (SEQ ID NO: 2), wherein the amino acid sequence is present immediately subsequent to position 453, relative to a reference sequence numbered according to the amino acid sequence of SEQ ID NO: 138. In some embodiments, an AAV capsid variant described herein comprises the amino acid sequence of HDSPHK (SEQ ID NO: 2), wherein the amino acid sequence is present immediately subsequent to position 453, relative to a reference sequence numbered according to the amino acid sequence of SEQ ID NO: 982.

In some embodiments, an AAV capsid variant described herein comprises (i) the amino acid sequence of HDSPHK (SEQ ID NO: 2), which is present immediately subsequent to position 453; and (ii) a deletion of amino acids SG at position 454 and 455; wherein (i) and (ii) are numbered according to SEQ ID NO: 138.

In some embodiments, an AAV capsid variant described herein comprises an amino acid other than S at position 454 and/or an amino acid other than G at position 455, numbered according to SEQ ID NO: 138. In some embodiments, the AAV capsid variant comprises the amino acid H at position 454 and the amino acid D at position 455, numbered according to SEQ ID NO: 138. In some embodiments, the AAV capsid variant further comprises the amino acid sequence of SPHSKA (SEQ ID NO: 941). In some embodiments, the AAV capsid variant comprises: (i) the amino acid H at position 454 and the amino acid D at position 455, and (ii) the amino acid sequence SPHSKA (SEQ ID NO: 941), wherein the amino acid sequence of SPHSKA (SEQ ID NO: 941) is present immediately subsequent to position 455, wherein (i) and (ii) are numbered according to SEQ ID NO: 138.

In some embodiments, an AAV capsid variant described herein comprises a modification, e.g., substitution, relative to SEQ ID NO: 138. In some embodiments, the AAV capsid variant comprises a modification, e.g., substitution, at position S454 and/or G455, numbered relative to SEQ ID NO: 138. In some embodiments, the AAV capsid variant comprises a S454H substitution and/or G455D substitution, numbered relative to SEQ ID NO: 138. In some embodiments, the AAV capsid variant comprises a S454H substitution and a G455D substitution, numbered relative to SEQ ID NO: 138. In some embodiments, the AAV capsid variant further comprises the amino acid sequence of SPHSKA (SEQ ID NO: 941). In some embodiments, the AAV capsid variant comprises: (i) a S454H substitution and a G455D substitution, and (ii) the amino acid sequence SPHSKA (SEQ ID NO: 941), wherein the amino acid sequence of SPHSKA (SEQ ID NO: 941) is present immediately subsequent to position 455, wherein (i) and (ii) are numbered according to SEQ ID NO: 138.

In some embodiments, the AAV capsid variant further comprises one, two, or all of an amino acid other than T at position 450 (e.g., S, Y, or G), an amino acid other than I at position 451 (e.g., M or L), and/or an amino acid other than N at position 452 (e.g., S), relative to a reference sequence numbered according to the amino acid sequence of SEQ ID NO: 138. In some embodiments, the AAV capsid variant further comprises an S at position 450 and an M at position 451, relative to a reference sequence numbered according to the amino acid sequence of SEQ ID NO: 138. In some embodiments, the AAV capsid variant further comprises a Y at position 450, an L at position 451, and an S at position 452, relative to a reference sequence numbered according to the amino acid sequence of SEQ ID NO: 138. In some embodiments, the AAV capsid variant further comprises a G at position 450, an L at position 451, and an S at position 452, relative to a reference sequence numbered according to the amino acid sequence of SEQ ID NO: 138.

In some embodiments, the AAV capsid variant further comprises one, two, three, four, or all of an amino acid other than Q at position 456 (e.g., R or L), N at position 457 (e.g., H, K, or R), Q at position 458 (e.g., R or T), Q at position 459 (H), and/or T at position 460 (N or S), relative to a reference sequence numbered according to the amino acid sequence of SEQ ID NO: 138. In some embodiments, the AAV capsid variant further comprises an R at position 456, relative to a reference sequence numbered according to the amino acid sequence of SEQ ID NO: 138. In some embodiments, the AAV capsid variant further comprises an L at position 456, relative to a reference sequence numbered according to the amino acid sequence of SEQ ID NO: 138. In some embodiments, the AAV capsid variant further comprises an H at position 457 and an R at position 458, relative to a reference sequence numbered according to the amino acid sequence of SEQ ID NO: 138. In some embodiments, the AAV capsid variant further comprises a K at position 457 and an N at position 460, relative to a reference sequence numbered according to the amino acid sequence of SEQ ID NO: 138. In some embodiments, the AAV capsid variant further comprises a T at position 458, an H at position 459, and an S at position 460, relative to a reference sequence numbered according to the amino acid sequence of SEQ ID NO: 138. In some embodiments, the AAV capsid variant further comprises an R at position 456, an R at position 457, and an R at position 458, relative to a reference sequence numbered according to the amino acid sequence of SEQ ID NO: 138.

In some embodiments, an capsid variant described herein comprises an amino acid other than I at position 451, an amino acid other than N at position 452, and an amino acid other than G at position 453, numbered according to SEQ ID NO: 138 or 981. In some embodiments, the AAV capsid variant comprises E at position 451, R at position 452, and V at position 453, numbered according to SEQ ID NO: 138 or 981. In some embodiments, the AAV capsid variant comprises the substitutions I451E, N452R, and G453V, numbered according to SEQ ID NO: 138 or 981.

In some embodiments, the AAV capsid variant comprises the amino acid sequence of SPHSKA (SEQ ID NO: 941), wherein the amino acid sequence is present immediately subsequent to position 455 and wherein the AAV capsid variant comprises the E at position 451, R at position 452, and V at position 453, numbered according to the amino acid sequence of SEQ ID NO: 138 or 981. In some embodiments, the AAV capsid variant comprises the substitutions I451E, N452R, and G453V, and further comprises the amino acid sequence of SPHSKA (SEQ ID NO: 941), wherein the amino acid sequence is present immediately subsequent to position 455, all numbered according to SEQ ID NO: 138 or 981. In some embodiments, the AAV capsid variant comprises the amino acid sequence of ERVSGSPHSKA (SEQ ID NO: 7581), and wherein the amino acid sequence is present immediately subsequent to position 449 and replaces positions 450-455, numbered according to SEQ ID NO: 138. In some embodiments, the AAV capsid variant comprises the amino acid sequence of KTERVSGSPHSKAQNQQT (SEQ ID NO: 3589), wherein the amino acid sequence is present immediately subsequent to position 448 and replaces positions 449-460, numbered according to SEQ ID NO: 138.

In some embodiments, an AAV capsid variant described herein comprises an amino acid other than T at position 450, an amino acid other than I at position 451, and an amino acid other than N at position 452, numbered according to SEQ ID NO: 138 or 982. In some embodiments, the AAV capsid variant comprises A at position 450, E at position 451, and I at position 452, numbered according to SEQ ID NO: 138 or 982. In some embodiments, the AAV capsid variant comprises the substitutions T450A, I451E, and N452I, numbered according to SEQ ID NO: 138 or 982.

In some embodiments, the AAV capsid variant comprises the amino acid sequence of SPHKSG (SEQ ID NO: 946), which is present immediately subsequent to positions 455, and further comprises A at position 450, E at position 451, I at position 452, H at position 454, and D at position 455, all numbered according to SEQ ID NO: 138 or 982. In some embodiments, the AAV capsid variant comprises the substitutions T450A, I451E, or N452I, and further comprises the amino acid sequence HDSPHK (SEQ ID NO: 2) present immediately subsequent to position 453, all numbered according to SEQ ID NO: 138 or 982. In some embodiments, the AAV capsid variant comprises the amino acid sequence of AEIGHDSPHKSG (SEQ ID NO: 7582), wherein the amino acid sequence is present immediately subsequent to position 449 and replaces positions 450-455, numbered according to SEQ ID NO: 138. In some embodiments, the AAV capsid variant comprises the amino acid sequence of KAEIGHDSPHKSGQNQQT (SEQ ID NO: 1754), wherein the amino acid sequence is present immediately subsequent to position 448 and replaces positions 449-460, numbered according to SEQ ID NO: 138.

In some embodiments, the AAV capsid variant, further comprises a substitution at position K449, e.g., a K449R substitution, numbered according to SEQ ID NO: 138. In some embodiments, the AAV capsid variant, further comprises an amino acid other than K at position 449 (e.g., R), relative to a reference sequence numbered according to the amino acid sequence of SEQ ID NO: 138. In some embodiments, the AAV capsid variant comprises an R at position 449, relative to a reference sequence numbered according to the amino acid sequence of SEQ ID NO: 138. In some embodiments, the AAV capsid variant further comprises a modification, e.g., an insertion, substitution, and/or deletion in loop I, II, VI, and/or VIII.

In some embodiments, the AAV capsid variant, further comprises an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, of the amino acid sequence of SEQ ID NO: 138. In some embodiments, the AAV capsid variant, further comprises an amino acid sequence comprising at least one, two or three, but not more than 30, 20 or 10 amino acids that differ from the amino acid sequence of SEQ ID NO: 138. In some embodiments, the AAV capsid variant further comprises the amino acid sequence of SEQ ID NO: 138, or an amino acid sequence with at least 70% (e.g., at least about 80, 85, 90, 95, 96, 97, 98, or 99%) sequence identity thereto.

In some embodiments, the AAV capsid variant further comprises (a) a VP1 protein comprising the amino acid sequence of SEQ ID NO: 138, 981, or 982; (b) a VP2 protein comprising the amino acid sequence of positions 138-736 of SEQ ID NO: 138 or positions 138-742 of SEQ ID NO: 981 or 982; (c) a VP3 protein comprising the amino acid sequence of positions 203-736 of SEQ ID NO: 138 or positions 203-742 of SEQ ID NO: 981 or 982; or (d) an amino acid sequence with at least 70% (e.g., at least about 80, 85, 90, 95, 96, 97, 98, or 99%) sequence identity to any of the amino acid sequences in (a)-(c), an amino acid sequence comprising at least one, two or three, but not more than 30, 20 or 10 different amino acids relative to any of the amino acid sequences in (a)-(c), or an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to any of the amino acid sequences in (a)-(c).

In some embodiments, the AAV capsid variant further comprises an amino acid sequence encoded by the nucleotide sequence of SEQ ID NO: 137, or a sequence with at least 70% (e.g., at least about 80, 85, 90, 95, 96, 97, 98, or 99%) sequence identity thereto. In some embodiments, the AAV capsid variant further comprises an amino acid sequence encoded by a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 137. In some embodiments, the AAV capsid variant further comprises an amino acid sequence encoded by a nucleotide sequence comprising at least one, two or three, but not more than 30, 20 or 10 different nucleotides, relative to the amino acid sequence of SEQ ID NO: 137.

In some embodiments, the nucleotide sequence encoding the AAV capsid variant further comprises the nucleotide sequence of SEQ ID NO: 137, or a sequence with at least 70% (e.g., at least about 80, 85, 90, 95, 96, 97, 98, or 99%) sequence identity thereto. In some embodiments, the nucleotide sequence encoding the AAV capsid variant further comprises a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 137. In some embodiments, the nucleotide sequence encoding the AAV capsid variant further comprises a nucleotide sequence comprising at least one, two or three, but not more than 30, 20 or 10 different nucleotides, relative to the amino acid sequence of SEQ ID NO: 137.

In some embodiments, an AAV capsid variant of the present disclosure comprises an amino acid sequence as described herein, e.g., an amino acid sequence of an AAV capsid variant of TTM-001 or TTM-002, e.g., as described in Tables 3 and 4. In some embodiments, an AAV capsid variant of the present disclosure comprises an amino acid sequence as described herein, e.g., an amino acid sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, or TTM-026, e.g., as described in Table 4.

In some embodiments, an AAV capsid variant described herein comprises a VP1, VP2, and/or VP3 protein comprising an amino acid sequence described herein, e.g., an amino acid sequence of an AAV capsid variant of TTM-001 or TTM-002, e.g., as described in Tables 3 and 4. In some embodiments, an AAV capsid variant described herein comprises a VP1, VP2, and/or VP3 protein comprising an amino acid sequence described herein, e.g., an amino acid sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, or TTM-026, e.g., as described in Table 4. In some embodiments, an AAV capsid variant described herein comprises an amino acid sequence encoded by a nucleotide sequence as described herein, e.g., a nucleotide sequence of an AAV capsid variant of TTM-001 or TTM-002, e.g., as described in Tables 3 and 5. In some embodiments, an AAV capsid variant described herein comprises an amino acid sequence encoded by a nucleotide sequence as described herein, e.g., a nucleotide sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, or TTM-026, e.g., as described in Table 5.

Sn some embodiments, a polynucleotide or nucleic acid encoding an AAV capsid variant, of the present disclosure comprises a nucleotide sequence described herein, e.g., a nucleotide sequence of an AAV capsid variant of TTM-001 or TTM-002, e.g., as described in Tables 3 and 5. In some embodiments, a polynucleotide or nucleic acid encoding an AAV capsid variant, of the present disclosure comprises a nucleotide sequence described herein, e.g., a nucleotide sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TTM-024, TTM-025, or TTM-026, e.g., as described in Table 5.

TABLE 3
Exemplary full length capsid sequences

| | VP1 | VP1 | Peptide | Peptide |
|---|---|---|---|---|
| | DNA SEQ | (amino acid) | (amino acid) | DNA SEQ |
| Name | ID NO: | SEQ ID NO: | SEQ ID NO: | ID NO: |
| TTM-001 | 983 | 981 | 941 | 942 |
| TTM-002 | 984 | 982 | 2 | 3 |

TABLE 4
Exemplary full length capsid amino acid sequences

| | SEQ | |
|---|---|---|
| Name and | ID | |
| Annotation | NO: | Amino Acid Sequence |
| TTM-001 | 981 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVEMIPQYGYLTLNDGSQAVGRS |
| position 455); | | SFYCLEYFPSQMLRTGNNFQFSYEFENVPFHSSYAHSQSLDRLMNPLIDQYLYYLS |
| 742 aa | | KTINGSGSPHSKAQNQQTLKFSVAGPSNMAVOGRNYIPGPSYRQQRVSTTVTONNN |
| | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-002 | 982 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 454 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 453); | | SFYCLEYFPSQMLRTGNNFQFSYEFENVPFHSSYAHSQSLDRLMNPLIDQYLYYLS |
| 742 aa | | KTINGHDSPHKSGQNQQTLKFSVAGPSNMAVOGRNYIPGPSYRQQRVSTTVTQNNN |
| | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-003 | 36 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTERVSGSPHSKAQNQQTLKFSVAGPSNMAVOGRNYIPGPSYRQQRVSTTVTONNN |
| positions 451, | | SEFAWPGASSWALNGRNSLMNPGPAMASHKEGEDRFFPLSGSLIFGKQGTGRDNVD |
| 452, and 453 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-004 | 37 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 454 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 453); | | SFYCLEYFPSQMLRTGNNFQFSYEFENVPFHSSYAHSQSLDRLMNPLIDQYLYYLS |
| modifications at | | KAEIGHDSPHKSGQNQQTLKFSVAGPSNMAVQGRNYIPGPSYRQQRVSTTVTQNNN |
| positions 450, | | SEFAWPGASSWALNGRNSLMNPGPAMASHKEGEDRFFPLSGSLIFGKQGTGRDNVD |
| 451, and 452 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSLITQYSTGQVSVEIEWELQKENSKRWNPEIQYTSNYYKSNNVEFAVNTEGVYS |
| | | EPRPIGTRYLTRNL |
| TTM-005 | 38 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTIIGSGSPHSKAQNRHTLKFSVAGPSNMAVOGRNYIPGPSYRQQRVSTTVTQNNN |
| positions 452, | | SEFAWPGASSWALNGRNSLMNPGPAMASHKEGEDRFFPLSGSLIFGKQGTGRDNVD |
| 464, and 465 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-006 | 39 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSEGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTEKMSGSPHSKAQNQQTLKFSVAGPSNMAVQGRNYIPGPSYRQQRVSTTVTQNNN |
| positions 451, | | SEFAWPGASSWALNGRNSLMNPGPAMASHKEGEDRFFPLSGSLIFGKQGTGRDNVD |
| 452, and 453 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-007 | 40 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KEINGRGSPHSKAQNQQTLKFSVAGPSNMAVQGRNYIPGPSYRQQRVSTTVTQNNN |
| positions 450 | | SEFAWPGASSWALNGRNSLMNPGPAMASHKEGEDRFFPLSGSLIFGKQGTGRDNVD |
| and 454 | | ADKVMITNEEEIKTTNPVATESYGQVATNHQSAQAQAQTGWVONQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-008 | 41 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTFNGSGSPHSKAPNLQTLKFSVAGPSNMAVQGRNYIPGPSYRQQRVSTTVTONNN |
| positions 451, | | SEFAWPGASSWALNGRNSLMNPGPAMASHKEGEDRFFPLSGSLIFGKQGTGRDNVD |
| 462, and 464 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-009 | 42 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSEGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTEKTSGSPHSKAQNQQTLKFSVAGPSNMAVOGRNYIPGPSYRQQRVSTTVTONNN |
| positions 451, | | SEFAWPGASSWALNGRNSLMNPGPAMASHKEGEDRFFPLSGSLIFGKQGTGRDNVD |
| 452, and 453 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-010 | 43 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTMNGHDSPHSKAQNQQTLKFSVAGPSNMAVQGRNYIPGPSYRQQRVSTTVTQNNN |
| positions 451, | | SEFAWPGASSWALNGRNSLMNPGPAMASHKEGEDRFFPLSGSLIFGKQGTGRDNVD |
| 454, and 455 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-011 | 44 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDOYLYYLS |
| modifications at | | KTIDGHDSPHSKAQNQQTLKFSVAGPSNMAVOGRNYIPGPSYRQQRVSTTVTQNNN |
| positions 452, | | SEFAWPGASSWALNGRNSLMNPGPAMASHKEGEDRFFPLSGSLIFGKQGTGRDNVD |
| 454, and 455 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-012 | 45 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTNNGHDSPHSKAQNQQTLKFSVAGPSNMAVQGRNYIPGPSYRQQRVSTTVTQNNN |
| positions 452, | | SEFAWPGASSWALNGRNSLMNPGPAMASHKEGEDRFFPLSGSLIFGKQGTGRDNVD |
| 454, and 455 | | ADKVMITNEEEIKTTNPVATESYGQVATNHQSAQAQAQTGWVQNQGILPGMVWQDR |
| underlined; | | DVYLOGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-013 | 46 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTQRKSGSPHSKAQNQQTLKFSVAGPSNMAVQGRNYIPGPSYRQQRVSTTVTQNNN |
| positions 451, | | SEFAWPGASSWALNGRNSLMNPGPAMASHKEGEDRFFPLSGSLIFGKQGTGRDNVD |
| 452, and 453 | | ADKVMITNEEEIKTTNPVATESYGQVATNHQSAQAQAQTGWVONQGILPGMVWQDR |
| underlined; | | DVYLOGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-014 | 47 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTINGSGSPHSKAQARKTLKFSVAGPSNMAVQGRNYIPGPSYRQQRVSTTVTQNNN |
| positions 463, | | SEFAWPGASSWALNGRNSLMNPGPAMASHKEGEDRFFPLSGSLIFGKQGTGRDNVD |
| 464, and 465 | | ADKVMITNEEEIKTTNPVATESYGQVATNHQSAQAQAQTGWVONQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-015 | 48 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KYIVGSGSPHSKAQNQQTLKFSVAGPSNMAVQGRNYIPGPSYRQQRVSTTVTQNNN |
| positions 450 | | SEFAWPGASSWALNGRNSLMNPGPAMASHKEGEDRFFPLSGSLIFGKQGTGRDNVD |
| and 452 | | ADKVMITNEEEIKTTNPVATESYGQVATNHQSAQAQAQTGWVONQGILPGMVWODR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-016 | 49 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTISKRGSPHSKAQNQQTLKFSVAGPSNMAVQGRNYIPGPSYRQQRVSTTVTONNN |
| positions 452, | | SEFAWPGASSWALNGRNSLMNPGPAMASHKEGEDRFFPLSGSLIFGKQGTGRDNVD |
| 453, and 454 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-017 | 50 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KGLGGSGSPHSKAQNQQTLKFSVAGPSNMAVQGRNYIPGPSYRQQRVSTTVTQNNN |
| positions 450, | | SEFAWPGASSWALNGRNSLMNPGPAMASHKEGEDRFFPLSGSLIFGKQGTGRDNVD |
| 451, and 452 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-018 | 51 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTINGHDSPHSKAQNLQTLKFSVAGPSNMAVOGRNYIPGPSYRQQRVSTTVTQNNN |
| positions 454, | | SEFAWPGASSWALNGRNSLMNPGPAMASHKEGEDRFFPLSGSLIFGKQGTGRDNVD |
| 455, and 464 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-019 | 52 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTVNGHDSPHSKAQNQQTLKFSVAGPSNMAVQGRNYIPGPSYRQQRVSTTVTQNNN |
| positions 451, | | SEFAWPGASSWALNGRNSLMNPGPAMASHKEGEDRFFPLSGSLIFGKQGTGRDNVD |
| 454, and 455 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-020 | 53 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTINGHDSPHSKALNQQTLKFSVAGPSNMAVQGRNYIPGPSYRQQRVSTTVTQNNN |
| positions 454, | | SEFAWPGASSWALNGRNSLMNPGPAMASHKEGEDRFFPLSGSLIFGKQGTGRDNVD |
| 455, and 462 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-021 | 54 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | NLTSTVQVFTDSDYQLPYVLGSAHEGCLPPFPADVFMIPQYGYLTLNDGSQAVGRS |
| subsequent to | | WGYFDFNRFHCHFSPRDWORLINNNWGFRPKRLNFKLFNIQVKEVTDNNGVKTIAN |
| position 455); | | NLTSTVQVFTDSDYQLPYVLGSAHEGCLPPFPADVFMIPQYGYLTLNDGSQAVGRS |
| modifications at | | SFYCLEYFPSQMLRTGNNFQFSYEFENVPFHSSYAHSQSLDRLMNPLIDQYLYYLS |
| positions 454, | | KTINGHDSPHSKAQNQQSLKFSVAGPSNMAVQGRNYIPGPSYRQQRVSTTVTQNNN |
| 455, and 466 | | SEFAWPGASSWALNGRNSLMNPGPAMASHKEGEDRFFPLSGSLIFGKQGTGRDNVD |
| underlined; | | ADKVMITNEEEIKTTNPVATESYGQVATNHQSAQAQAQTGWVQNQGILPGMVWQDR |
| 742 aa | | DVYLOGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-022 | 55 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTINGHDSPHSKAQNQQILKFSVAGPSNMAVQGRNYIPGPSYRQQRVSTTVTQNNN |
| positions 454, | | SEFAWPGASSWALNGRNSLMNPGPAMASHKEGEDRFFPLSGSLIFGKQGTGRDNVD |
| 455, and 466 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-023 | 56 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| 742 aa | | KTINGSGSPHFTRQNQQTLKFSVAGPSNMAVQGRNYIPGPSYRQQRVSTTVTONNN |
| | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVONQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-024 | 57 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSEGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modifications at | | KTSNGHDSPHSKAQNQQTLKFSVAGPSNMAVQGRNYIPGPSYRQQRVSTTVTQNNN |
| positions 451, | | SEFAWPGASSWALNGRNSLMNPGPAMASHKEGEDRFFPLSGSLIFGKQGTGRDNVD |
| 454, and 455 | | ADKVMITNEEEIKTTNPVATESYGQVATNHQSAQAQAQTGWVONQGILPGMVWODR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-025 | 58 | MAADGYLPDWLEDNLSEGIREWWALKPGAPQPKANQQHODNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modification at | | KTINGSGSPHSLPWNQQTLKFSVAGPSNMAVQGRNYIPGPSYRQQRVSTTVTQNNN |
| position 462 | | SEFAWPGASSWALNGRNSLMNPGPAMASHKEGEDRFFPLSGSLIFGKOGTGRDNVD |
| underlined; | | ADKVMITNEEEIKTTNPVATESYGQVATNHQSAQAQAQTGWVQNQGILPGMVWQDR |
| 742 aa | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-026 | 59 | MAADGYLPDWLEDNLSEGIREWWALKPGAPQPKANQQHQDNARGLVLPGYKYLGPG |
| 6 mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at | | KRLNFGQTGDTESVPDPQPIGEPPAAPSGVGSLTMASGGGAPVADNNEGADGVGSS |
| position 456 | | SGNWHCDSQWLGDRVITTSTRTWALPTYNNHLYKQISNSTSGGSSNDNAYFGYSTP |
| (immediately | | WGYFDFNRFHCHFSPRDWORLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| position 455); | | SFYCLEYFPSQMLRTGNNFQFSYEFENVPFHSSYAHSQSLDRLMNPLIDQYLYYLS |
| modification at | | KTINGHDSPHSKAQNHQTLKFSVAGPSNMAVOGRNYIPGPSYRQQRVSTTVTQNNN |
| positions 454, | | SEFAWPGASSWALNGRNSLMNPGPAMASHKEGEDRFFPLSGSLIFGKQGTGRDNVD |
| 455, and 464 | | ADKVMITNEEEIKTTNPVATESYGQVATNHQSAQAQAQTGWVONQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |

TABLE 5
Exemplary full length capsid nucleic acid sequences

| | SEQ | |
|---|---|---|
| Name and | ID | |
| Annotation | NO: | NT Sequence |
| TTM-001 | 983 | ATGGCTGCCGATGGTTATCTTCCAGattggcTCGAGGACAACCTTAGTGAAGGAAT |
| 9 mer peptide | | TCGCGAGTGGTGGGCTTTGAAACCTGGAGCCCCTCAACCCAAGGCAAATCAACAAC |
| underlined | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGACCAATACTTGTAtTActTgagt |
| | | AAaACaATTAACGGAAGCGGAAGCCCACACAGCAAAGCACAAAACCAACAGACCET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTaTAA |
| TTM-002 | 984 | ATGGCTGCCGATGGTTATCTTCCAGattggcTCGAGGACAACCTTAGTGAAGGAAT |
| 7 mer peptide | | TCGCGAGTGGTGGGCTTTGAAACCTGGAGCCCCTCAACCCAAGGCAAATCAACAAC |
| underlined | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGACCAATACTTGTAtTActTgagt |
| | | AAaACaATTAACGGACACGACAGCCCACACAAAAGCGGACAAAACCAACAGACCET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTaTAA |
| TTM-023 | 6553 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGAGCGTGTGTCTGGTTCTCCGCATTCTAAGGCGCAGAATCAGCAGACGtT |
| | | gAAgTTttcgGTaGCtGGtCCtAGcAAcATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-004 | 6554 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAt TActTgagt |
| | | AAGGCGGAGATTGGTCATGATTCTCCGCATAAGTCTGGTCAGAATCAGCAGACGET |
| | | gAAgTTttcgGTaGCtGGtCCtAGCAAcATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTAATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAtTAACgaGgAActTa |
| TTM-005 | 6555 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTATTGGTTCTGGTTCTCCGCATTCTAAGGCGCAGAATCGTCATACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-006 | 6556 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGAGAAGATGTCTGGTTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-007 | 16 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGGAGATTAATGGTCGTGGTTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-008 | 17 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | CACCCGAACCTGGGCCCTGCCCACCTACAACAATCACCTCTACAAGCAAATCTCCA |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGTTTAATGGTTCTGGTTCTCCGCATTCTAAGGCGCCGAATCTGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-009 | 18 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGAGAAGACGTCTGGTTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-010 | 19 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATGAATGGTCATGATTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-011 | 20 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCITTCCATAGCAGCTACGCTCACAGCC |
| | | AAAGCCTGGACCGACTAATGAATCCACTCATCGAtCAgTAtcTGTAtTActTgagt |
| | | AAGACGATTGATGGTCATGATTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-012 | 21 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGITCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGAATAATGGTCATGATTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-013 | 22 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGCAGCGTAAGTCTGGTTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-014 | 23 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTTCTGGTTCTCCGCATTCTAAGGCGCAGGCGCGTAAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-015 | 24 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGTATATTGTGGGTTCTGGTTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-016 | 25 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTTCTAAGCGTGGTTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-017 | 26 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGGGTCTGGGTGGTTCTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | GCGGACAAAGTCATGATAACCAACGAAGAAGAAATTAAAACTACTAACCCGGTAGC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-018 | 27 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCTGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAtTAACgaGgAActTa |
| TTM-019 | 28 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGTGAATGGTCATGATTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-020 | 29 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCTGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-021 | 30 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGITTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCAGCAGTCTET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-022 | 31 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | GCTCGGGTCGGCTCACGAGGGCTGCCTCCCGCCGTTCCCAGCGGACGTTTTCATGA |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCAGCAGATTET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-023 | 32 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTTCTGGTTCTCCGCATTTTACGCGTCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-024 | 33 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGTCTAATGGTCATGATTCTCCGCATTCTAAGGCGCAGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-025 | 34 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAGACGATTAATGGTTCTGGTTCTCCGCATTCTCTGCCGTGGAATCAGCAGACGET |
| | | AAAGCCTGGACCGACTAATGAATCCACTCATCGAtCAgTAtcTGTAtTActTgagt |
| | | AAGACGATTAATGGTTCTGGTTCTCCGCATTCTCTGCCGTGGAATCAGCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-026 | 35 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCATCAGACGET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCITTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |

In some embodiments, the polynucleotide encoding an AAV capsid variant, described herein comprises the nucleotide sequence of SEQ ID NO: 983 or 984, or a nucleotide sequence with at least 70% (e.g., at least about 80, 85, 90, 95, 96, 97, 98, or 99%) sequence identity thereto.

In some embodiments, the polynucleotide encoding an AAV capsid variant, described herein comprises the nucleotide sequence of any one of SEQ ID NOs: 6553-6556 or 16-35, or a nucleotide sequence with at least 70% (e.g., at least about 80, 85, 90, 95, 96, 97, 98, or 99%) sequence identity thereto.

In some embodiments, the polynucleotide encoding an AAV capsid variant described herein comprises the nucleotide sequence of SEQ ID NO: 983, or a nucleotide sequence with at least 70% (e.g., at least about 80, 85, 90, 95, 96, 97, 98, or 99%) sequence identity thereto. In some embodiments, the nucleotide sequence encoding an AAV capsid variant described herein, comprises a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 983. In some embodiments, the nucleotide sequence encoding an AAV capsid variant described herein, comprises a nucleotide sequence comprising at least one, two or three, but not more than 30, 20 or 10 different nucleotides relative to the amino acid sequence of SEQ ID NO: 983. In some embodiments, the nucleic acid sequence encoding an AAV capsid variant described herein is codon optimized.

In some embodiments, the polynucleotide encoding an AAV capsid variant described herein comprises the nucleotide sequence of SEQ ID NO: 984, or a nucleotide sequence with at least 70% (e.g., at least about 80, 85, 90, 95, 96, 97, 98, or 99%) sequence identity thereto. In some embodiments, the nucleotide sequence encoding an AAV capsid variant described herein, comprises a nucleotide sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the nucleotide sequence of SEQ ID NO: 984. In some embodiments, the nucleotide sequence encoding an AAV capsid variant described herein, comprises a nucleotide sequence comprising at least one, two or three, but not more than 30, 20 or 10 different nucleotides, relative to the amino acid sequence of SEQ ID NO: 984. In some embodiments, the nucleic acid sequence encoding an AAV capsid variant described herein is codon optimized.

In some embodiments, an AAV capsid variant described herein comprises the amino acid sequence of SEQ ID NO: 36-59, 981, or 982, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 36-59, 981, or 982. In some embodiments, the AAV capsid variant, comprises an amino acid sequence comprising at least one, two or three, but not more than 30, 20 or 10 different amino acids, relative to the amino acid sequence of SEQ ID NO: 36-59, 981, or 982.

In some embodiments, an AAV capsid variant described herein comprises the amino acid sequence of SEQ ID NO: 36, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 36. In some embodiments, the AAV capsid variant, comprises an amino acid sequence comprising at least one, two or three, but not more than 30, 20 or 10 different amino acids, relative to the amino acid sequence of SEQ ID NO: 36.

In some embodiments, an AAV capsid variant described herein comprises the amino acid sequence of SEQ ID NO: 39, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 39. In some embodiments, the AAV capsid variant, comprises an amino acid sequence comprising at least one, two or three, but not more than 30, 20 or 10 different amino acids, relative to the amino acid sequence of SEQ ID NO: 39.

In some embodiments, an AAV capsid variant described herein comprises the amino acid sequence of SEQ ID NO: 51, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 51. In some embodiments, the AAV capsid variant, comprises an amino acid sequence comprising at least one, two or three, but not more than 30, 20 or 10 different amino acids, relative to the amino acid sequence of SEQ ID NO: 51.

In some embodiments, an AAV capsid variant described herein comprises the amino acid sequence of SEQ ID NO: 52, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 52. In some embodiments, the AAV capsid variant, comprises an amino acid sequence comprising at least one, two or three, but not more than 30, 20 or 10 different amino acids, relative to the amino acid sequence of SEQ ID NO: 52.

In some embodiments, an AAV capsid variant described herein, comprises the amino acid sequence of SEQ ID NO: 981, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 981. In some embodiments, an AAV capsid variant described herein comprises an amino acid sequence comprising at least one, two or three, but not more than 30, 20 or 10 different amino acids, relative to the amino acid sequence of SEQ ID NO: 981.

In some embodiments, an AAV capsid variant described herein comprises the amino acid sequence of SEQ ID NO: 982, or an amino acid sequence with at least 70% (e.g., at least about 80, 85, 90, 95, 96, 97, 98, or 99%) sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 982. In some embodiments, the AAV capsid variant, comprises an amino acid sequence comprising at least one, two or three, but not more than 30, 20 or 10 different amino acids, relative to the amino acid sequence of SEQ ID NO: 982.

In some embodiments, an AAV capsid variant described herein comprises an amino acid sequence encoded by the nucleotide sequence of SEQ ID NO: 983 or 984, or a nucleotide sequence with at least 70% (e.g., at least about 80, 85, 90, 95, 96, 97, 98, or 99%) sequence identity thereto. In some embodiments, an AAV capsid variant described herein comprises an amino acid sequence encoded by a nucleotide sequence comprising at least one, two or three, but not more than 30, 20 or 10 different nucleotides, relative to the amino acid sequence of SEQ ID NO: 983 or 984. In some embodiments, an AAV capsid variant described herein comprises an amino acid sequence encoded by a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 983 or 984.

In some embodiments, an AAV capsid variant described herein comprises an amino acid sequence encoded by the nucleotide sequence of any one of SEQ ID NOs: 6553-6556 or 16-35, or a nucleotide sequence with at least 70% (e.g., at least about 80, 85, 90, 95, 96, 97, 98, or 99%) sequence identity thereto. In some embodiments, an AAV capsid variant described herein comprises an amino acid sequence encoded by a nucleotide sequence comprising at least one, two or three, but not more than 30, 20 or 10 different nucleotides, relative to the amino acid sequence of any one of SEQ ID NOs: 6553-6556 or 16-35. In some embodiments, an AAV capsid variant described herein comprises an amino acid sequence encoded by a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of any one of SEQ ID NOs: 6553-6556 or 16-35.

In some embodiments, an AAV capsid variant described herein comprises a VP1, VP2, VP3 protein, or a combination thereof. In some embodiments, an AAV capsid variant comprises the amino acid sequence corresponding to positions 138-742, e.g., a VP2, of SEQ ID NO: 981 or 982, or a sequence with at least 70% (e.g., at least about 80, 85, 90, 95, 96, 97, 98, or 99%) sequence identity thereto. In some embodiments, the AAV capsid protein comprises the amino acid sequence corresponding to positions 203-742, e.g., a VP3, of SEQ ID NO: 981 or 982, or a sequence with at least 70% (e.g., at least about 80, 85, 90, 95, 96, 97, 98, or 99%) sequence identity thereto. In some embodiments, the AAV capsid variant comprises the amino acid sequence corresponding to positions 1-742, e.g., a VP1, of SEQ ID NO: 981 or 982, or an amino acid sequence with at least 70% (e.g., at least about 80, 85, 90, 95, 96, 97, 98, or 99%) sequence identity thereto.

In some embodiments, an AAV capsid variant described herein comprises a VP1, VP2, VP3 protein, or a combination thereof. In some embodiments, an AAV capsid variant comprises the amino acid sequence corresponding to positions 138-742, e.g., a VP2, of any one of SEQ ID NOs: 36-59, or a sequence with at least 70% (e.g., at least about 80, 85, 90, 95, 96, 97, 98, or 99%) sequence identity thereto. In some embodiments, the AAV capsid protein comprises the amino acid sequence corresponding to positions 203-742, e.g., a VP3, of any one of SEQ ID NOs: 36-59, or a sequence with at least 70% (e.g., at least about 80, 85, 90, 95, 96, 97, 98, or 99%) sequence identity thereto. In some embodiments, the AAV capsid variant comprises the amino acid sequence corresponding to positions 1-742, e.g., a VP1, of any one of SEQ ID NOs: 36-59, or an amino acid sequence with at least 70% (e.g., at least about 80, 85, 90, 95, 96, 97, 98, or 99%) sequence identity thereto.

In some embodiments, an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid variant described herein transduces a brain region, e.g., a midbrain region (e.g., the hippocampus, or thalamus) or the brain stem. In some embodiments, the level of transduction is at least 5, 10, 15, 20, 25, 30, 35, 40, 45, 50, 55, 60, or 65-fold greater as compared to a reference sequence of SEQ ID NO: 138. In some embodiments, the level of transduction is at least 30, 35, 40, 45, 50, 55, 60, or 65-fold greater as compared to a reference sequence of SEQ ID NO: 138.

In some embodiments, an AAV capsid variant described herein is enriched at least about 3, 4, 5, 6, 7, 8, 9, or 10-fold in the brain compared to a reference sequence of SEQ ID NO: 138. In some embodiments, an AAV capsid variant described herein is enriched at least about 20, 25, 30, 35, 40, 45, 50, 55, 60, 65, 70, 75, 80 or 85-fold in the brain compared to a reference sequence of SEQ ID NO: 138.

In some embodiments, an AAV capsid variant described herein is enriched in the brain of at least two to three species, e.g., a non-human primate and rodent (e.g., mouse) species, compared to a reference sequence of SEQ ID NO: 138. In some embodiments, an AAV capsid variant described herein is enriched at least about 10, 15, 20, 25, 30, 35, 40, 45, 50, 55, 60, 65, 70, 75, 80, 85, 90, 95, or 100-fold in the brain of at least two to three species, e.g., a non-human primate and rodent (e.g., mouse) species, compared to a reference sequence of SEQ ID NO: 138. In some embodiments, the at least two to three species are Macaca fascicularis, Chlorocebus sabaeus, Callithrix jacchus, and/or mouse (e.g., BALB/c mice, C57Bl/6 mice, and/or CD-1 outbred mice).

In some embodiments, an AAV capsid variant described herein is enriched at least about 2, 2.5, 3, 3.5, 4, 4.5, 5, 5.5, 6, 6.5, 7, 7.5, or 8-fold, in the brain compared to a reference sequence of SEQ ID NO: 981. In some embodiments, an AAV capsid variant described herein is enriched about 2, 2.5, 3, 3.5, 4, 4.5, 5, or 5.5-fold, in the brain compared to a reference sequence of SEQ ID NO: 982.

In some embodiments, an AAV capsid variant described herein delivers an increased level of viral genomes to a brain region. In some embodiments, the level of viral genomes is increased by at least 20, 25, 30, 35, 40, 45, or 50-fold, as compared to a reference sequence of SEQ ID NO: 138. In some embodiments, the brain region comprises a midbrain region (e.g., the hippocampus or thalamus) and/or the brainstem.

In some embodiments, an AAV capsid variant described herein delivers an increased level of a payload to a brain region. In some embodiments, the level of the payload is increased by at least 20, 25, 30, 35, 40, 45, 50, 55, 60, 65, or 70-fold, as compared to a reference sequence of SEQ ID NO: 138. In some embodiments, the brain region comprises a midbrain region (e.g., the hippocampus or thalamus) and/or the brainstem.

In some embodiments, an AAV capsid variant described herein is enriched at least about 5, 10, 15, 20, 25, 30, or 35-fold, in the spinal cord compared to a reference sequence of SEQ ID NO: 138.

In some embodiments, an AAV capsid variant described herein shows preferential transduction in a brain region relative to the transduction in the dorsal root ganglia (DRG). In some embodiments, the AAV capsid variant shows preferential transduction in a brain region relative to the transduction in the liver. In some embodiments, the AAV capsid variant shows preferential transduction in a brain region relative to the transduction in the liver and the DRG. In some embodiments, the AAV capsid variant shows preferential transduction in a brain region relative to the transduction in the heart. In some embodiments, the AAV capsid variant shows preferential transduction in a brain region relative to the transduction in the heart and DRG. In some embodiments, the AAV capsid variant shows preferential transduction in a brain region relative to the transduction in the heart, DRG, and liver.

In some embodiments, an AAV capsid variant described herein is capable of transducing non-neuronal cells, e.g., glial cells (e.g., oligodendrocytes or astrocytes). In some embodiments, the AAV capsid variant described herein is capable of transducing neuronal cells and non-neuronal cells, e.g., glial cells (e.g., oligodendrocytes or astrocytes). In some embodiments, the non-neuronal cells are glial cells, oligodendrocytes (e.g., Olig2 positive oligodendrocytes), or astrocytes (e.g., Olig2 positive astrocytes). In some embodiments, the AAV capsid variant is capable of transducing Olig2 positive cells, e.g., Olig2 positive astrocytes or Olig2 positive oligodendrocytes.

In some embodiments, an AAV capsid variant of the present disclosure has decreased tropism for the liver. In some embodiments, an AAV capsid variant comprises a modification, e.g., substitution (e.g., conservative substitution), insertion, or deletion, that results in reduced tropism (e.g., de-targeting) and/or activity in the liver. In some embodiments, the reduced tropism in the liver is compared to an otherwise similar capsid that does not comprise the modification, e.g., a wild-type capsid. In some embodiments, an AAV capsid variant described herein comprises a modification, e.g., substitution (e.g., conservative substitution), insertion, or deletion that results in one or more of the following properties: (1) reduced tropism in the liver; (2) reduced, e.g., de-targeted, expression in the liver; (3) reduced activity in the liver; and/or (4) reduced binding to galactose. In some embodiments, the reduction in any one, or all of properties (1)-(3) is compared to an otherwise similar AAV capsid variant that does not comprise the modification. Exemplary modifications are provided in WO 2018/119330; Pulicherla et al. (2011) Mol. Ther. 19(6): 1070-1078; Adachi et al. (2014) Nature Communications 5(3075), DOI: 10.1038/ncomms4075; and Bell et al. (2012) J. Virol. 86(13): 7326-33; the contents of which are hereby incorporated by reference in their entirety. In some embodiments, the AAV capsid variant comprises a modification e.g., substitution (e.g., conservative substitution), insertion, or deletion, at position N470 (e.g., N470A), D271 (e.g., D271A), N272 (e.g., N272A), Y446 (e.g., Y446A), N498 (e.g., N498Y or N4981), W503 (e.g., W503R or W503A), L620 (e.g., L620F), or a combination thereof, relative to a reference sequence numbered according to SEQ ID NO: 138. In some embodiments, the AAV capsid variant comprises one, two, three, four, five or all of an amino acid other than N at position 470 (e.g., A), an amino acid other than D at position 271 (e.g., A), an amino acid other than N at position 272 (e.g., A), an amino acid other than Y at position 446 (e.g., A), and amino acid other than N at position 498 (e.g., Y or I), and amino acid other than W at position 503 (e.g., R or A), and amino acid other than L at position 620 (e.g., F), relative to a reference sequence numbered according to SEQ ID NO: 138. In some embodiments, the AAV capsid variant comprises a modification e.g., substitution (e.g., conservative substitution), insertion, or deletion, at position N470 (e.g., N470A), D271 (e.g., D271A), N272 (e.g., N272A), Y446 (e.g., Y446A), and W503 (e.g., W503R or W503A), relative to a reference sequence numbered according to SEQ ID NO: 138. In some embodiments, the AAV capsid variant comprises a modification e.g., substitution (e.g., conservative substitution), insertion, or deletion, at N498 (e.g., N498Y) and L620 (e.g., L620F).

In some embodiments, an AAV capsid variant comprised herein comprises a modification as described in Adachi et al. (2014) Nature Communications 5(3075), DOI: 10.1038/ncomms4075, the contents of which are hereby incorporated by reference in its entirety. Exemplary modifications that alter or do not alter tissue transduction in at least the brain, liver, heart, lung, and/or kidney can be found in Supplementary Data 2 showing the AAV Barcode-Seq data obtained with AAV9-AA-VBCLib of Adachi et al. (supra), the contents of which are hereby incorporated by reference in its entirety.

In some embodiments, an AAV capsid variant, of the present disclosure is isolated, e.g., recombinant. In some embodiments, a polynucleotide encoding an AAV capsid polypeptide, e.g., an AAV capsid variant, of the present disclosure is isolated, e.g., recombinant.

Also provided herein are polynucleotide sequences encoding any of the AAV capsid variants described above and AAV particles, vectors, and cells comprising the same.

### Genetic Element

In some embodiments, an AAV particle, e.g., an AAV particle for the vectorized delivery of an antibody molecule described herein (e.g., an anti-HER2 antibody molecule), comprises a genetic element.

In some embodiments, the genetic element comprises a nucleotide sequence encoding a transgene, wherein the transgene encodes a payload, e.g., an antibody molecule (e.g., an anti-HER2 antibody molecule described herein). In some embodiments, the genetic element further comprises an inverted terminal repeat (ITR) region, a promoter, an enhancer, an intron region, an exon region, a nucleic acid encoding a transgene encoding a payload, a poly A signal region, or a combination thereof. In some embodiments, a genetic element comprises a 5′AAV ITR and/or a 3′ AAV ITR. In some embodiments, any or all components of the genetic elements can be modified or optimized to improve expression or targeting of the payload (e.g., an antibody molecule) encoded by the transgene.

### Genetic Element Component: Inverted Terminal Repeats (ITRs)

In some embodiments, the genetic element comprises an ITR region. In some embodiments, the genetic element comprises at least one ITR region and a nucleic acid encoding a transgene encoding a payload, e.g., an antibody molecule (e.g., an anti-HER2 antibody molecule). In some embodiments, the genetic element comprises two ITRs. In some embodiments, the two ITRs flank the nucleic acid encoding the transgene at the 5′ and 3′ ends. In some embodiments, the ITR functions as an origin of replication comprising recognition sites for replication. In some embodiments, the ITRs comprise sequence regions which can be complementary and symmetrically arranged. In some embodiments, the ITR incorporated into a genetic element may be comprised of naturally occurring nucleotide sequences or recombinantly derived nucleotide sequences.

In some embodiments, the ITR may be of the same AAV serotype as the capsid, e.g., a capsid protein selected from any of the AAV serotypes listed in Table 1, or a functional variant thereof. In some embodiments, the ITR may be of a different AAV serotype than the capsid protein. In some embodiments, the AAV particle comprises a genetic element comprising two ITRs wherein the two ITRs of the genetic element are of the same AAV serotype. In other embodiments, the two ITRs of a genetic element are of different AAV serotypes. In some embodiments both ITRs of the genetic element of the AAV particle are AAV2 ITRs or a functional variant thereof.

In some embodiments, the ITR may be about 100 to about 150 nucleotides in length. In some embodiments, the ITR comprises about 120-140 nucleotides in length, e.g., about 130 nucleotides in length. In some embodiments, the ITR comprises about 140-150 nucleotides in length, about 141 nucleotides in length. In some embodiments, the genetic element comprises an ITR region comprising the nucleotide sequence of any of the sequences provided in Table 7 or a nucleotide sequence with at least 70%, 75%, 80%, 85%, 90%, 95% or 99% sequence identity thereto. In some embodiments, the genetic element comprises two ITR regions comprising the nucleotide sequence of any of the sequences provided in Table 7 or a nucleotide sequence with at least 70%, 75%, 80%, 85%, 90%, 95% or 99% sequence identity thereto, wherein the first and second ITR comprise the same sequence or wherein the first and second ITR comprise different sequences.

TABLE 7
Inverted Terminal Repeat (ITR) Sequence Regions

| Sequence | | SEQ | |
|---|---|---|---|
| Region | | ID | |
| Name | Length | NO | Sequence |
| ITR1 | 130 | 6559 | ctgcgcgctcgctcgctcactgagg |
| | | | ccgcccgggcaaagcccgggcgtcg |
| | | | ggcgacctttggtcgcccggcctca |
| | | | gtgagcgagcgagcgcgcagagagg |
| | | | gagtggccaactccatcactagggg |
| | | | ttcct |
| ITR2 | 141 | 6560 | cctgcaggcagctgcgcgctcgctc |
| | | | gctcactgaggccgcccgggcaaag |
| | | | cccgggcgtcgggcgacctttggtc |
| | | | gcccggcctcagtgagcgagcgagc |
| | | | gcgcagagagggagtggccaactcc |
| | | | atcactaggggttcct |
| ITR3 | 130 | 6561 | aggaacccctagtgatggagttggc |
| | | | cactccctctctgcgcgctcgctcg |
| | | | ctcactgaggccgggcgaccaaagg |
| | | | tcgcccgacgcccgggctttgcccg |
| | | | ggcggcctcagtgagcgagcgagcg |
| | | | cgcag |
| ITR4 | 141 | 6562 | aggaacccctagtgatggagttggc |
| | | | cactccctctctgcgcgctcgctcg |
| | | | ctcactgaggccgggcgaccaaagg |
| | | | tcgcccgacgcccgggctttgcccg |
| | | | ggcggcctcagtgagcgagcgagcg |
| | | | cgcagctgcctgcagg |

In some embodiment, the genetic element comprises an ITR provided in Table 7. In some embodiments, the genetic element comprises an ITR chosen from any one of ITR1-ITR4 or a functional variant thereof. In some embodiments, the genetic element comprises ITR1. In some embodiments, the genetic element comprises ITR1. In some embodiments, the ITR comprises the nucleotide sequence of any one of SEQ ID NOs: 6559-6562 as provided in Table 7, or a sequence with at least 70%, 75%, 80%, 85%, 90%, 95% or 99% sequence identity thereto. In some embodiments, the ITR comprises the nucleotide sequence of SEQ ID NO: 6559 or a nucleotide sequence with at least 70%, 75%, 80%, 85%, 90%, 95% or 99% sequence identity thereto. In some embodiments, the ITR comprises the nucleotide sequence of SEQ ID NO: 6561 or a nucleotide sequence with at least 70%, 75%, 80%, 85%, 90%, 95% or 99% sequence identity thereto.

In some embodiments, the genetic element comprises ITR1 and ITR2. In some embodiments, the genetic element comprises ITR1 at the 5′ end and ITR2 at the 3′ end. In some embodiments, the genetic element comprises an ITR, e.g., a 5′ ITR, comprising the nucleotide sequence of SEQ ID NO: 6559 or a sequence with at least with at least 70%, 75%, 80%, 85%, 90%, 95% or 99% sequence identity thereto; and an ITR, e.g., a 3′ ITR, comprising the nucleotide sequence of SEQ ID NO: 6561 or a nucleotide sequence at least 70%, 75%, 80%, 85%, 90%, 95% or 99% sequence identity thereto.

### Genetic Element Component: Promoters

In some embodiments, the genetic element comprises an element to enhance the transgene target specificity and/or expression (See e.g., Powell et al. Viral Expression Cassette Elements to Enhance Transgene Target Specificity and Expression in Gene Therapy, 2015; the contents of which are herein incorporated by reference in its entirety). In some embodiments, an element to enhance the transgene target specificity and/or expression comprise a promoter, an enhancer, e.g., a CMV enhancer, or both. In some embodiments, the genetic element comprises a promoter operably linked to a transgene encoded by a nucleic acid molecule encoding a payload, e.g., antibody molecule (e.g., an anti-HER2 antibody molecule). In some embodiments, the genetic element comprises an enhancer, e.g., a CMV enhancer. In some embodiment, the genetic element comprises at least two promoters.

In some embodiments, the genetic element comprises a promoter that is species specific, inducible, tissue-specific, and/or cell cycle-specific (e.g., as described in Parr et al., Nat. Med. 3:1145-9 (1997); the contents of which are herein incorporated by reference in their entirety). In some embodiments, the genetic element comprises a promoter that is sufficient for expression, e.g., in a target cell, of a payload (e.g., an antibody molecule, e.g., an anti-HER2 molecule) encoded by a transgene.

In some embodiments, the promoter may be a naturally occurring promoter, or a non-naturally occurring promoter. In some embodiments, the promoter is from a naturally expressed protein. In some embodiments, the promoter is an engineered promoter. In some embodiments, the promoter comprises a viral promoter, plant promoter, and/or a mammalian promoter. In some embodiments, the promoter may be a human promoter. In some embodiments, the promoter may be truncated. In some embodiments, the promoter is not a cell specific promoter.

In some embodiments, the genetic element comprises a promoter that results in expression in one or more, e.g., multiple, cells and/or tissues, e.g., a ubiquitous promoter. In some embodiments, a promoter that results in expression in one or more tissues includes but is not limited to a human elongation factor 1α-subunit (EF1α) promoter, a cytomegalovirus (CMV) immediate-early enhancer and/or promoter, a chicken β-actin (CBA) promoter and its derivative CAG, a β glucuronidase (GUSB) promoter, or ubiquitin C (UBC) promoter. In some embodiments, a tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes. In some embodiments, the genetic element comprises a ubiquitous promoter as described in Yu et al. (Molecular Pain 2011, 7:63), Soderblom et al. (E. Neuro 2015), Gill et al., (Gene Therapy 2001, Vol. 8, 1539-1546), and Husain et al. (Gene Therapy 2009), each of which are incorporated by reference in their entirety. In some embodiments, the genetic element comprises a ubiquitous promoter chosen from CMV, CBA (including derivatives CAG, CB6, CBh, etc.), EF-1α, PGK, UBC, GUSB (hGBp), or UCOE (promoter of HNRPA2B1-CBX3).

In some embodiments, the genetic element comprises a muscle-specific promoter, e.g., a promoter that results in expression in a muscle cell. In some embodiments, a muscle-specific promoter includes but is not limited to a mammalian muscle creatine kinase (MCK) promoter, a mammalian desmin (DES) promoter, a mammalian troponin I (TNNI2) promoter, a synthetic C5-12 promoter, and a mammalian skeletal alpha-actin (ASKA) promoter (see, e.g. U.S. Patent Publication US20110212529, the contents of which are herein incorporated by reference in their entirety).

In some embodiments, the genetic element comprises a nervous system specific promoter, e.g., a promoter that results in expression of a payload in a neuron, an astrocyte, and/or an oligodendrocyte. In some embodiments, a nervous system specific promoter that results in expression in neurons includes but is not limited to a neuron-specific enolase (NSE) promoter, a platelet-derived growth factor (PDGF) promoter, a platelet-derived growth factor B-chain (PDGF-β) promoter, a synapsin (Syn) promoter, a methyl-CpG binding protein 2 (MeCP2) promoter, a Ca²⁺/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, a enkephalin (Enk) promoter, and an excitatory amino acid transporter 2 (EAAT2) promoter. In some embodiments, a nervous system specific promoter that results in expression in astrocytes includes but is not limited to a glial fibrillary acidic protein (GFAP) promoter and an EAAT2 promoter. In some embodiments, a nervous system specific promoter that results in expression in oligodendrocytes includes but is not limited to a myelin basic protein (MBP) promoter. In some embodiments, the genetic element comprises a nervous system specific promoter as described in Husain et al. (Gene Therapy 2009), Passini and Wolfe (J. Virol. 2001, 12382-12392), Xu et al. (Gene Therapy 2001, 8, 1323-1332), Drews et al. (Mamm Genome (2007) 18:723-731), and Raymond et al. (Journal of Biological Chemistry (2004) 279(44) 46234-46241), each of which are incorporated by reference in their entirety.

In some embodiments, the genetic element comprises a liver promoter, e.g. a promoter that results in expression a liver cell. In some embodiments, the liver promoter is chosen from human α-1-antitrypsin (hAAT) or thyroxine binding globulin (TBG). In some embodiments, the genetic element comprises an RNA pol III promoter. In some embodiments, the RNA pol III promoter is chosen from U6 or H1. In some embodiments, the genetic element comprises an endothelial cell promoter, e.g., a promoter that results in expression in an endothelial cell. In some embodiments, the endothelial cell promoter is an intercellular adhesion molecule 2 (ICAM2) promoter.

In some embodiments, the genetic element comprises a promoter chosen from a CAG promoter, a CBA promoter (e.g., a minimal CBA promoter), a CB promoter, a CMV(IE) promoter and/or enhancer, a GFAP promoter, a synapsin promoter, an ICAM2 promoter, or a functional variant thereof. In some embodiments, the genetic element comprises a CAG promoter, a CMVie enhancer, and a minimal CBA promoter. In some embodiments, the genetic element comprises a CMV(IE) promoter and a CB promoter.

In some embodiments, the CAG promoter comprises the nucleotide sequence of SEQ ID NO: 6563 or nucleotide sequence with at least 70%, 75%, 80%, 85%, 90%, 95%, or 99% sequence identity thereto. In some embodiments, the CBA promoter (e.g., a minimal CBA promoter) comprises the nucleotide sequence of SEQ ID NO: 6565 or nucleotide sequence with at least 70%, 75%, 80%, 85%, 90%, 95%, or 99% sequence identity thereto. In some embodiments, the CB promoter comprises the nucleotide sequence of SEQ ID NO: 6566 or nucleotide sequence with at least 70%, 75%, 80%, 85%, 90%, 95%, or 99% sequence identity thereto. In some embodiments, the GFAP promoter comprises the nucleotide sequence of SEQ ID NO: 6568 or nucleotide sequence with at least 70%, 75%, 80%, 85%, 90%, 95%, or 99% sequence identity thereto. In some embodiments, the synapsin promoter comprises the nucleotide sequence of SEQ ID NO: 6569 or nucleotide sequence with at least 70%, 75%, 80%, 85%, 90%, 95%, or 99% sequence identity thereto. In some embodiments, the CMV(IE) promoter comprises the nucleotide sequence of SEQ ID NO: 6594 or nucleotide sequence with at least 70%, 75%, 80%, 85%, 90%, 95%, or 99% sequence identity thereto. In some embodiments, the CMV(ie) enhancer comprises the nucleotide sequence of SEQ ID NO: 6564 or nucleotide sequence with at least 70%, 75%, 80%, 85%, 90%, 95%, or 99% sequence identity thereto.

In some embodiments, the genetic element comprises a promoter provided in Table 8. In some embodiments, the promoter is chosen from any one of Promoter 1-Promoter 12, or a functional variant thereof. In some embodiments, the promoter comprises the nucleotide sequence of any one of SEQ ID NOs: 6563-6572, or 6593-6594, or a sequence with at least 70%, 75%, 80%, 85%, 90%, 95% or 99% sequence identity thereto. In some embodiments, the promoter comprises the nucleotide sequence of SEQ ID NO: 6563 or a sequence with at least 70%, 75%, 80%, 85%, 90%, 95% or 99% sequence identity thereto. In some embodiments, the promoter comprises the nucleotide sequence of SEQ ID NO: 6564, or a sequence with at least 70%, 75%, 80%, 85%, 90%, 95% or 99% sequence identity thereto. In some embodiments, the promoter comprises the nucleotide sequence of SEQ ID NO: 6565, or a sequence with at least 70%, 75%, 80%, 85%, 90%, 95% or 99% sequence identity thereto. In some embodiments, the promoter comprises the nucleotide sequence of SEQ ID NO: 6566, or a sequence with at least 70%, 75%, 80%, 85%, 90%, 95% or 99% sequence identity thereto. In some embodiments, the promoter comprises the nucleotide sequence of SEQ ID NO: 6568, or a sequence with at least 70%, 75%, 80%, 85%, 90%, 95% or 99% sequence identity thereto. In some embodiments, the promoter comprises the nucleotide sequence of SEQ ID NO: 6569, or a sequence with at least 70%, 75%, 80%, 85%, 90%, 95% or 99% sequence identity thereto. In some embodiments, the promoter comprises the nucleotide sequence of SEQ ID NO: 6594, or a sequence with at least 70%, 75%, 80%, 85%, 90%, 95% or 99% sequence identity thereto. In some embodiments, the promoter comprises the nucleotide sequence of SEQ ID NO: 4599, or a sequence with at least 70%, 75%, 80%, 85%, 90%, 95% or 99% sequence identity thereto.

TABLE 8
Promoter Sequence Regions

| | SEQ | |
|---|---|---|
| | ID | Promoter Sequence Regions |
| Name | NO | Sequence |
| Promoter 1 | 6563 | CTAGTTATTAATAGTAATCAATTACGGGGTCATTAGTTCATAGCCCATATATGGAGTTCC |
| | | GCGTTACATAACTTACGGTAAATGGCCCGCCTGGCTGACCGCCCAACGACCCCCGCCCAT |
| | | TGACGTCAATAATGACGTATGTTCCCATAGTAACGCCAATAGGGACTTTCCATTGACGTC |
| | | AATGGGTGGAGTATTTACGGTAAACTGCCCACTTGGCAGTACATCAAGTGTATCATATGC |
| | | CAAGTACGCCCCCTATTGACGTCAATGACGGTAAATGGCCCGCCTGGCATTATGCCCAGT |
| | | ACATGACCTTATGGGACTTTCCTACTTGGCAGTACATCTACGTATTAGTCATCGCTATTA |
| | | CCATGGTCGAGGTGAGCCCCACGTTCTGCTTCACTCTCCCCATCTCCCCCCCCTCCCCAC |
| | | CCCCAATTTTGTATTTATTTATTTTTTAATTATTTTGTGCAGCGATGGGGGCGGGGGGGG |
| | | GGGGGGGGCGCGCGCCAGGCGGGGCGGGGCGGGGCGAGGGGCGGGGCGGGGCGAGGCGGA |
| | | GAGGTGCGGCGGCAGCCAATCAGAGCGGCGCGCTCCGAAAGTTTCCTTTTATGGCGAGGC |
| | | GGCGGCGGCGGCGGCCCTATAAAAAGCGAAGCGCGCGGCGGGCGGGAGTCGCTGCGCGCT |
| | | GCCTTCGCCCCGTGCCCCGCTCCGCCGCCGCCTCGCGCCGCCCGCCCCGGCTCTGACTGA |
| | | CCGCGTTACTCCCACAGGTGAGCGGGCGGGACGGCCCTTCTCCTCCGGGCTGTAATTAGC |
| | | GCTTGGTTTAATGACGGCTTGTTTCTTTTCTGTGGCTGCGTGAAAGCCTTGAGGGGCTCC |
| | | GGGAGGGCCCTTTGTGCGGGGGGAGCGGCTCGGGGGGTGCGTGCGTGTGTGTGTGCGTGG |
| | | GGAGCGCCGCGTGCGGCTCCGCGCTGCCCGGCGGCTGTGAGCGCTGCGGGCGCGGCGCGG |
| | | GGCTTTGTGCGCTCCGCAGTGTGCGCGAGGGGAGCGCGGCCGGGGGCGGTGCCCCGCGGT |
| | | GCGGGGGGGGCTGCGAGGGGAACAAAGGCTGCGTGCGGGGTGTGTGCGTGGGGGGGTGAG |
| | | CAGGGGGTGTGGGCGCGTCGGTCGGGCTGCAACCCCCCCTGCACCCCCCTCCCCGAGTTG |
| | | CTGAGCACGGCCCGGCTTCGGGTGCGGGGCTCCGTACGGGGCGTGGCGCGGGGCTCGCCG |
| | | TGCCGGGCGGGGGGTGGCGGCAGGTGGGGGTGCCGGGGGGGGGGGGGCCGCCTCGGGCCG |
| | | GGGAGGGCTCGGGGGAGGGGCGCGGCGGCCCCCGGAGCGCCGGCGGCTGTCGAGGCGCGG |
| | | CGAGCCGCAGCCATTGCCTTTTATGGTAATCGTGCGAGAGGGCGCAGGGACTTCCTTTGT |
| | | CCCAAATCTGTGCGGAGCCGAAATCTGGGAGGCGCCGCCGCACCCCCTCTAGCGGGCGCG |
| | | GGGCGAAGCGGTGCGGCGCCGGCAGGAAGGAAATGGGGGGGGAGGGCCTTCGTGCGTCGC |
| | | CGCGCCGCCGTCCCCTTCTCCCTCTCCAGCCTCGGGGCTGTCCGCGGGGGGACGGCTGCC |
| | | TTCGGGGGGGACGGGGCAGGGCGGGGTTCGGCTTCTGGCGTGTGACCGGCGGCTCTAGAG |
| | | CCTCTGCTAACCATGTTCATGCCTTCTTCTTTTTCCTACAGCTCCTGGGCAACGTGCTGG |
| | | TTATTGTGCTGTCTCATCATTTTGGCAAAGAATTC |
| Promoter 2 | 6564 | TAGCCCATATATGGAGTTCCGCGTTACATAACTTACGGTAAATGGCCCGCCTGGCTGACC |
| | | GCCCAACGACCCCCGCCCATTGACGTCAATAATGACGTATGTTCCCATAGTAACGCCAAT |
| | | AGGGACTTTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTAAATGGCC |
| | | CGCCTGGCATTATGCCCAGTACATGACCTTATGGGACTTTCCTACTTGGCAGTACATCT |
| Promoter 3 | 6565 | CATGGTCGAGGTGAGCCCCACGTTCTGCTTCACTCTCCCCATCTCCCCCCCCTCCCCACC |
| | | CCCAATTTTGTATTTATTTATTTTTTAATTATTTTGTGCAGCGATGGGGGCGGGGGGGGG |
| | | GGGGGGGCGCGCGCCAGGCGGGGCGGGGCGGGGCGAGGGGCGGGGCGGGGCGAGGCGGAG |
| | | AGGTGCGGCGGCAGCCAATCAGAGCGGCGCGCTCCGAAAGTTTCCTTTTATGGCGAGGCG |
| | | GCGGCGGCGGCGGCCCTATAAAAAGCGAAGCGCGCGGCGGGCG |
| Promoter 4 | 6566 | CCACGTTCTGCTTCACTCTCCCCATCTCCCCCCCCTCCCCACCCCCAATTTTGTATTTAT |
| | | TTATTTTTTAATTATTTTGTGCAGCGATGGGGGCGGGGGGGGGGGGCGCGCGCCAGGCGG |
| | | GGCGGGGCGGGGCGAGGGGGGGGGCGGGGCGAGGCGGAGAGGTGCGGCGGCAGCCAATCA |
| | | GAGCGGCGCGCTCCGAAAGTTTCCTTTTATGGCGAGGCGGCGGCGGCGGCGGCCCTATAA |
| | | AAAGCGAAGCGCGCGGCGGG |
| Promoter 5 | 6567 | CGACATTGATTATTGACTAGTTATTAATAGTAATCAATTACGGGGTCATTAGTTCATAGC |
| | | CCATATATGGAGTTCCGCGTTACATAACTTACGGTAAATGGCCCGCCTGGCTGACCGCCC |
| | | AACGACCCCCGCCCATTGACGTCAATAATGACGTATGTTCCCATAGTAACGCCAATAGGG |
| | | ACTTTCCATTGACGTCAATGGGTGGAGTATTTACGGTAAACTGCCCACTTGGCAGTACAT |
| | | CAAGTGTATCATATGCCAAGTACGCCCCCTATTGACGTCAATGACGGTAAATGGCCCGCC |
| | | TGGCATTATGCCCAGTACATGACCTTATGGGACTTTCCTACTTGGCAGTACATCTACGTA |
| | | TTAGTCATCGCTATTACCATGTCGAGGCCACGTTCTGCTTCACTCTCCCCATCTCCCCCC |
| | | CCTCCCCACCCCCAATTTTGTATTTATTTATTTTTTAATTATTTTGTGCAGCGATGGGGG |
| | | CGGGGGGGGGGGGCGCGCGCCAGGCGGGGCGGGGCGGGGCGAGGGGCGGGGCGGGGCGAG |
| | | GCGGAGAGGTGCGGCGGCAGCCAATCAGAGCGGCGCGCTCCGAAAGTTTCCTTTTATGGC |
| | | GAGGCGGCGGCGGCGGCGGCCCTATAAAAAGCGAAGCGCGCGGCGGGCGGGAGC |
| Promoter 6 | 6568 | GATCTAACATATCCTGGTGTGGAGTAGCGGACGCTGCTATGACAGAGGCTCGGGGGCCTG |
| | | AGCTGGCTCTGTGAGCTGGGGAGGAGGCAGACAGCCAGGCCTTGTCTGCAAGCAGACCTG |
| | | GCAGCATTGGGCTGGCCGCCCCCCAGGGCCTCCTCTTCATGCCCAGTGAATGACTCACCT |
| | | TGGCACAGACACAATGTTCGGGGTGGGCACAGTGCCTGCTTCCCGCCGCACCCCAGCCCC |
| | | CCTCAAATGCCTTCCGAGAAGCCCATTGAGCAGGGGGCTTGCATTGCACCCCAGCCTGAC |
| | | AGCCTGGCATCTTGGGATAAAAGCAGCACAGCCCCCTAGGGGCTGCCCTTGCTGTGTGGC |
| | | GCCACCGGCGGTGGAGAACAAGGCTCTATTCAGCCTGTGCCCAGGAAAGGGGATCAGGGG |
| | | ATGCCCAGGCATGGACAGTGGGTGGCAGGGGGGGAGAGGAGGGCTGTCTGCTTCCCAGAA |
| | | GTCCAAGGACACAAATGGGTGAGGGGAGAGCTCTCCCCATAGCTGGGCTGCGGCCCAACC |
| | | CCACCCCCTCAGGCTATGCCAGGGGGTGTTGCCAGGGGCACCCGGGCATCGCCAGTCTAG |
| | | CCCACTCCTTCATAAAGCCCTCGCATCCCAGGAGCGAGCAGAGCCAGAGCAGGTTGGAGA |
| | | GGAGACGCATCACCTCCGCTGCTCGCGGGGATCCTCTAG |
| Promoter 7 | 6569 | TAGTATCTGCAGAGGGCCCTGCGTATGAGTGCAAGTGGGTTTTAGGACCAGGATGAGGCG |
| | | GGGTGGGGGTGCCTACCTGACGACCGACCCCGACCCACTGGACAAGCACCCAACCCCCAT |
| | | TCCCCAAATTGCGCATCCCCTATCAGAGAGGGGGAGGGGAAACAGGATGCGGCGAGGCGC |
| | | GTGCGCACTGCCAGCTTCAGCACCGCGGACAGTGCCTTCGCCCCCGCCTGGCGGCGCGCG |
| | | CCACCGCCGCCTCAGCACTGAAGGCGCGCTGACGTCACTCGCCGGTCCCCCGCAAACTCC |
| | | CCTTCCCGGCCACCTTGGTCGCGTCCGCGCCGCCGCCGGCCCAGCCGGACCGCACCACGC |
| | | GAGGCGCGAGATAGGGGGGCACGGGCGCGACCATCTGCGCTGCGGCGCCGGCGACTCAGC |
| | | GCTGCCTCAGTCTGCGGTGGGCAGCGGAGGAGTCGTGTCGTGCCTGAGAGCGCAGCTGTG |
| | | CTCCTGGGCACCGCGCAGTCCGCCCCCGCGGCTCCTGGCCAGACCACCCCTAGGACCCCC |
| | | TGCCCCAAGTCGCAGCC |
| Promoter 8 | 6570 | CTAGTCGACATTGATTATTGACTAGTTATTAATAGTAATCAATTACGGGGTCATTAGTTC |
| | | ATAGCCCATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTAACGCCAA |
| | | TAGGGACTTTCCATTGACGTCAATGGGTGGAGTATTTACGGTAAACTGCCCACTTGGCAG |
| | | TACATCAAGTGTATCATATGCCAAGTACGCCCCCTATTGACGTCAATGACGGTAAATGGC |
| | | CCGCCTGGCATTATGCCCAGTACATGACCTTATGGGACTTTCCTACTTGGCAGTACATCT |
| | | ACGTATTAGTCATCGCTATTAC |
| Promoter 9 | 6571 | CTAGTCGACATTGATTATTGACTAGTTATTAATAGTAATCAATTACGGGGTCATTAGTTC |
| | | ATAGCCCATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTAACGCCAA |
| | | TAGGGACTTTCCATTGACGTCAATGGGTGGAGTATTTACGGTAAACTGCCCACTTGGCAG |
| | | TACATCAAGTGTATCATATGCCAAGTACGCCCCCTATTGACGTCAATGACGGTAAATGGC |
| | | CCGCCTGGCATTATGCCCAGTACATGACCTTATGGGACTTTCCTACTTGGCAGTACATCT |
| | | ACGTATTAGTCATCGCTATTACCATGGTCGAGGTGAGCCCCACGTTCTGCTTCACTCTCC |
| | | CCATCTCCCCCCCCTCCCCACCCCCAATTTTGTATTTATTTATTTTTTAATTATTTTGTG |
| | | CAGCGATGGGGGCGGGGGGGGGGGGGGGGCGCGCGCCAGGCGGGCGGGGGCGGGGCGAGG |
| | | GGCGGGCGGGGGCGAGGCGGAGAGGTGCGGCGGCAGCCAATCAGAGCGGCGCGCTCCGAA |
| | | AGTTTCCTTTTATGGCGAGGCGGCGGCGGCGGCGGCCCTATAAAAAGCGAAGCGCGCGGC |
| | | GGGCGGGAGTCGCTGCGCGCTGCCTTCGCCCCGTGCCCCGCTCCGCCGCCGCCTCGCGCC |
| | | GCCCGCCCCGGCTCTGACTGACCGCGTTACTCCCACAGGTGAGCGGGCGGGACGGCCCTT |
| | | CTCCTCCGGGCTGTAATTAGCGCTTGGTTTAATGACGGCTTGTTTCTTTTCTGTGGCTGC |
| | | GTGAAAGCCTTGAGGGGCTCCGGGAGGGCCCTTTGTGCGGGGGGAGCGGCTCGGGGGGTG |
| | | CGTGCGTGTGTGTGTGCGTGGGGAGCGCCGCGTGCGGCTCCGCGCTGCCCGGCGGCTGTG |
| | | AGCGCTGCGGGCGCGGCGCGGGGCTTTGTGCGCTCCGCAGTGTGCGCGAGGGGAGCGCGG |
| | | CCGGGGGCGGTGCCCCGCGGTGCGGGGGGGGCTGCGAGGGGAACAAAGGCTGCGTGCGGG |
| | | GTGTGTGCGTGGGGGGGTGAGCAGGGGGTGTGGGCGCGTCGGTCGGGCTGCAACCCCCCC |
| | | TGCACCCCCCTCCCCGAGTTGCTGAGCACGGCCCGGCTTCGGGTGCGGGGCTCCGTACGG |
| | | GGCGTGGCGCGGGGCTCGCCGTGCCGGGCGGGGGGTGGCGGCAGGTGGGGGTGCCGGGCG |
| | | GGGCGGGGCCGCCTCGGGCCGGGGAGGGCTCGGGGGAGGGGCGCGGCGGCCCCCGGAGCG |
| | | CCGGCGGCTGTCGAGGCGCGGCGAGCCGCAGCCATTGCCTTTTATGGTAATCGTGCGAGA |
| | | GGGCGCAGGGACTTCCTTTGTCCCAAATCTGTGCGGAGCCGAAATCTGGGAGGCGCCGCC |
| | | GCACCCCCTCTAGCGGGCGCGGGGCGAAGCGGTGCGGCGCCGGCAGGAAGGAAATGGGCG |
| | | GGGAGGGCCTTCGTGCGTCGCCGCGCCGCCGTCCCCTTCTCCCTCTCCAGCCTCGGGGCT |
| | | GTCCGCGGGGGGACGGCTGCCTTCGGGGGGGACGGGGCAGGGCGGGGTTCGGCTTCTGGC |
| | | GTGTGACCGGCGGCTCTAGAGCCTCTGCTAACCATGTTCATGCCTTCTTCTTTTTCCTAC |
| | | AGCTCCTGGGCAACGTGCTGGTTATTGTGCTGTCTCATCATTTTGGCAAAGAATTC |
| Promoter | 6572 | CTAGTCGACATTGATTATTGACTAGTTATTAATAGTAATCAATTACGGGGTCATTAGTTC |
| 10 | | ATAGCCCATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTAACGCCAA |
| | | TAGGGACTTTCCATTGACGTCAATGGGTGGAGTATTTACGGTAAACTGCCCACTTGGCAG |
| | | TACATCAAGTGTATCATATGCCAAGTACGCCCCCTATTGACGTCAATGACGGTAAATGGC |
| | | CCGCCTGGCATTATGCCCAGTACATGACCTTATGGGACTTTCCTACTTGGCAGTACATCT |
| | | ACGTA |
| Promoter | 6593 | CTAGTTATTAATAGTAATCAATTACGGGGTCATTAGTTCATAGCCCATATATGGAGTTCC |
| 11 | | GCGTTACATAACTTACGGTAAATGGCCCGCCTGGCTGACCGCCCAACGACCCCCGCCCAT |
| | | TGACGTCAATAATGACGTATGTTCCCATAGTAACGCCAATAGGGACTTTCCATTGACGTC |
| | | AATGGGTGGAGTATTTACGGTAAACTGCCCACTTGGCAGTACATCAAGTGTATCATATGC |
| | | CAAGTACGCCCCCTATTGACGTCAATGACGGTAAATGGCCCGCCTGGCATTATGCCCAGT |
| | | ACATGACCTTATGGGACTTTCCTACTTGGCAGTACATCTACGTATTAGTCATCGCTATTA |
| | | CCATGGTCGAGGTGAGCCCCACGTTCTGCTTCACTCTCCCCATCTCCCCCCCCTCCCCAC |
| | | CCCCAATTTTGTATTTATTTATTTTTTAATTATTTTGTGCAGCGATGGGGGCGGGGGGGG |
| | | GGGGGGGGCGCGCGCCAGGCGGGGCGGGGCGGGGCGAGGGGCGGGGCGGGGCGAGGCGGA |
| | | GAGGTGCGGCGGCAGCCAATCAGAGCGGCGCGCTCCGAAAGTTTCCTTTTATGGCGAGGC |
| | | GGCGGCGGCGGCGGCCCTATAAAAAGCGAAGCGCGCGGCGGGCGGGAGTCGCTGCGCGCT |
| | | GCCTTCGCCCCGTGCCCCGCTCCGCCGCCGCCTCGCGCCGCCCGCCCCGGCTCTGACTGA |
| | | CCGCGTTACTCCCACAGGTGAGCGGGCGGGACGGCCCTTCTCCTCCGGGCTGTAATTAGC |
| | | GCTTGGTTTAATGACGGCTTGTTTCTTTTCTGTGGCTGCGTGAAAGCCTTGAGGGGCTCC |
| | | GGGAGGGCCCTTTGTGCGGGGGGAGCGGCTCGGGGGGTGCGTGCGTGTGTGTGTGCGTGG |
| | | GGAGCGCCGCGTGCGGCTCCGCGCTGCCCGGCGGCTGTGAGCGCTGCGGGCGCGGCGCGG |
| | | GGCTTTGTGCGCTCCGCAGTGTGCGCGAGGGGAGCGCGGCCGGGGGCGGTGCCCCGCGGT |
| | | GCGGGGGGGGCTGCGAGGGGAACAAAGGCTGCGTGCGGGGTGTGTGCGTGGGGGGGTGAG |
| | | CAGGGGGTGTGGGCGCGTCGGTCGGGCTGCAACCCCCCCTGCACCCCCCTCCCCGAGTTG |
| | | CTGAGCACGGCCCGGCTTCGGGTGCGGGGCTCCGTACGGGGCGTGGCGCGGGGCTCGCCG |
| | | TGCCGGGCGGGGGGTGGCGGCAGGTGGGGGTGCCGGGGGGGGCGGGGCCGCCTCGGGCCG |
| | | GGGAGGGCTCGGGGGAGGGGCGCGGCGGCCCCCGGAGCGCCGGCGGCTGTCGAGGCGCGG |
| | | CGAGCCGCAGCCATTGCCTTTTATGGTAATCGTGCGAGAGGGCGCAGGGACTTCCTTTGT |
| | | CCCAAATCTGTGCGGAGCCGAAATCTGGGAGGCGCCGCCGCACCCCCTCTAGCGGGCGCG |
| | | GGGCGAAGCGGTGCGGCGCCGGCAGGAAGGAAATGGGGGGGGAGGGCCTTCGTGCGTCGC |
| | | CGCGCCGCCGTCCCCTTCTCCCTCTCCAGCCTCGGGGCTGTCCGCGGGGGGACGGCTGCC |
| | | TTCGGGGGGGACGGGGCAGGGCGGGGTTCGGCTTCTGGCGTGTGACCGGCGGCTCTAGAG |
| | | CCTCTGCTAACCATGTTCATGCCTTCTTCTTTTTCCTACAGCTCCTGGGCAACGTGCTGG |
| | | TTATTGTGCTGTCTCATCATTTTGGCAAAGAATT |
| Promoter | 6594 | GACATTGATTATTGACTAGTTATTAATAGTAATCAATTACGGGGTCATTAGTTCATAGCC |
| 12 | | CATATATGGAGTTCCGCGTTACATAACTTACGGTAAATGGCCCGCCTGGCTGACCGCCCA |
| | | ACGACCCCCGCCCATTGACGTCAATAATGACGTATGTTCCCATAGTAACGCCAATAGGGA |
| | | CTTTCCATTGACGTCAATGGGTGGAGTATTTACGGTAAACTGCCCACTTGGCAGTACATC |
| | | AAGTGTATCATATGCCAAGTACGCCCCCTATTGACGTCAATGACGGTAAATGGCCCGCCT |
| | | GGCATTATGCCCAGTACATGACCTTATGGGACTTTCCTACTTGGCAGTACATCTACGTAT |
| | | TAGTCATCGCTATTACCATG |

In some embodiments, the genetic element comprises Promoter 1. In some embodiments, the genetic element comprises Promoter 2. In some embodiments, the genetic element comprises Promoter 3. In some embodiments, the genetic element comprises Promoter 4. In some embodiments, the genetic element comprises Promoter 5. In some embodiments, the genetic element comprises Promoter 6. In some embodiments, the genetic element comprises Promoter 12.

In some embodiments, the promoter of the genetic element further comprises at least one promoter sub-region. In some embodiments, the promoter comprises Promoter 1, further comprising Promoter 2 and Promoter 3 sub-regions. In some embodiments, the genetic element comprises at least 2 or more promoters. In some embodiments, the genetic element comprises Promoter 12 and Promoter 4.

### Genetic Element Component: Untranslated Regions (UTRs)

In some embodiments, the genetic element comprises an untranslated region (UTR). In some embodiments, a wild-type UTR of a gene are transcribed but not translated. In some embodiments, the 5′ UTR starts at the transcription start site and ends at the start codon and the 3′ UTR starts immediately following the stop codon and continues until the termination signal for transcription.

In some embodiments, a UTR comprises a feature found in abundantly expressed genes of specific target organs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the genetic element of an AAV particle described herein to enhance expression in hepatic cell lines or liver.

In some embodiments, a genetic element comprises a 5′UTR, e.g., a wild-type (e.g., naturally occurring) 5′UTR or a recombinant (e.g., non-naturally occurring) 5′UTR. In some embodiments, a 5′ UTR comprises a feature which plays a role in translation initiation. In some embodiments, a UTR, e.g., a 5′ UTR, comprises a Kozak sequence. In some embodiments, a Kozak sequence is involved in the process by which the ribosome initiates translation of many genes. In some embodiments, a Kozak sequence has the consensus sequence of CCR(A/G)CCAUGG, where R is apurine (adenine or guanine) three bases upstream of the start codon (ATG), which is followed by another ‘G’. In some embodiments, a Kozak sequence comprises the nucleotide sequence of GAGGAGCCACC (SEQ ID NO: 4543) or a nucleotide sequence with at least 95-99% sequence identity thereto. In some embodiments, a Kozak sequence comprises the nucleotide sequence of GCCGCCACCATG (SEQ ID NO: 6589), or a nucleotide sequence with at least 95-99% sequence identity thereto. In some embodiments, a genetic element comprises a 5′UTR comprising a Kozak sequence. In some embodiments, a genetic element comprises a 5′UTR that does not comprise a Kozak sequence.

In some embodiments, the genetic element comprises a 3′UTR, e.g., a wild-type (e.g., naturally occurring) 3′UTR or a recombinant (e.g., non-naturally occurring) 3′UTR. In some embodiments, a 3′ UTR comprises an element that modulates, e.g., increases or decreases, stability of a nucleic acid. In some embodiments, a 3′ UTR comprises stretches of Adenosines and Uridines embedded therein, e.g., an AU rich signature. These AU rich signatures are generally prevalent in genes with high rates of turnover and are described, e.g., in Chen et al, 1995, the contents of which are herein incorporated by reference in its entirety. In some embodiments, an AR rich signature comprises an AU rich element (ARE). In some embodiments, a 3′UTR comprises an ARE chosen from a class I ARE (e.g., c-Myc and MyoD), a class II ARE (e.g., GM-CSF and TNF-α), a class TUI ARE (e.g., c-Jun and Myogenin), or combination thereto. Tn some embodiments, a class I ARE comprises several dispersed copies of an AUUUA motif within U-rich regions. In some embodiments, a class II ARE comprises two or more overlapping UUAUUUA(U/A)(U/A) nonamers. In some embodiments, a class III ARE comprises U rich regions and/or do not contain an AUUUA motif. In some embodiments, an ARE destabilizes the messenger.

In some embodiments, a 3′UTR comprises a binding site for a protein member of the ELAV family. In some embodiments, a 3′ UTR comprises a binding site for an HuR protein. In some embodiments, an HuR protein binds to an ARE of any one of classes I-III and/or increases the stability of mRNA. Without wishing to be bound by theory, it is believed in some embodiments, that a 3′UTR comprising an HuR specific binding sites will lead to HuR binding and, stabilization of a message in vivo.

In some embodiments, the 3′ UTR of the genetic element comprises an oligo(dT) sequence for templated addition of a poly-A tail.

In some embodiments, the genetic element comprises a miRNA seed, binding site and/or full sequence. Generally, microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, the microRNA sequence comprises a seed region, e.g., a sequence in the region of positions 2-8 of the mature microRNA, which sequence has perfect Watson-Crick complementarity to the miRNA target sequence of the nucleic acid. In some embodiments, the genetic element or viral genome may be engineered to include, alter or remove at least one miRNA binding site, sequence, or seed region.

In some embodiments, a UTR from any gene known in the art may be incorporated into the genetic element of an AAV particle described herein. These UTRs, or portions thereof, may be placed in the same orientation as in the gene from which they were selected, or they may be altered in orientation or location. In some embodiments, the UTR used in the genetic element of the AAV particle may be inverted, shortened, lengthened, made with one or more other 5′ UTRs or 3′ UTRs known in the art. In some embodiments, an altered UTR, comprises a UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. In some embodiments, the genetic element comprises an artificial UTR, e.g., a UTR that is not a variant of a wild-type, e.g., a naturally occurring, UTR. In some embodiments, the genetic element comprises a UTR selected from a family of transcripts whose proteins share a common function, structure, feature or property.

### Genetic Element Component: miR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the genetic element of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a genetic element of an AAV particle described herein in a cell or tissue where the corresponding mRNA is expressed.

In some embodiments, the genetic element of an AAV particle described herein comprises a nucleotide sequence encoding a microRNA binding site, e.g., a detargeting site. In some embodiments, the genetic element of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the genetic element (e.g., 3′ relative to the nucleotide sequence encoding a payload), e.g., before the polyA sequence, 5′-UTR region of the genetic element (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complimentary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous.

In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complimentary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 4673), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4673, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCACACTCC A (SEQ ID NO: 4674), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4674, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 4675), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4675, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4679), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4679, e.g., wherein the modification can result in a mismatch between the encoded miR binding site and the corresponding miRNA. In some embodiments, the genetic element comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions, relative to the nucleotide sequence of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 4676), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4676, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g. an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 4677), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4677, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the spacer sequence comprises one or more of (i) GGAT; (ii) CACGTG; (iii) GCATGC, or a repeat of one or more of (i)-(iii).

In certain embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 4678), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4678, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR142 binding site, a miR183 binding site, a miR182 binding site, a miR 96 binding site, a mir-1 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, an encoded miR binding site series comprises at least 2-5 copies (e.g., 2 or 3 copies) of a combination of at least two, three, four, five, or all of a miR122 binding site, a miR142 binding site, a miR183 binding site, a miR182 binding site, a miR96 binding site, and/or a mir-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

### Genetic Element Component: Polyadenylation Sequence

In some embodiments, the genetic element of the AAV particles of the present disclosure comprise a polyadenylation sequence. In some embodiments, the genetic element comprises a polyadenylation (referred to herein as poly A, polyA, or poly-A) sequence between the 3′ end of the transgene encoding the payload and the 5′ end of the 3′ITR. In some embodiments, the genetic element comprises two or more polyA sequences. In some embodiments, the genetic element does not comprise a polyA sequence.

In some embodiments, the genetic element comprises a rabbit globin polyA signal region. In some embodiments, the rabbit globin polyA signal region comprises the nucleotide sequence of SEQ ID NO: 6590, or a nucleotide sequence with at least 70%, 75%, 80%, 85%, 90%, 95%, or 99% sequence identity thereto.

TABLE 13
PolyA Signal Sequence Regions

| | SEQ ID | |
|---|---|---|
| Name | NO | Sequence |
| PolyA1 | 6590 | GATCTTTTTCCCTCTGCCAAAAATTATGGGGACATCATGAAGCCCCTTGAGCATCTGA |
| | | CTTCTGGCTAATAAAGGAAATTTATTTTCATTGCAATAGTGTGTTGGAATTTTTTGTG |
| | | TCTCTCACTCG |
| PolyA2 | 6591 | GGGTGGCATCCCTGTGACCCCTCCCCAGTGCCTCTCCTGGCCCTGGAAGTTGCCACTC |
| | | CAGTGCCCACCAGCCTTGTCCTAATAAAATTAAGTTGCATCATTTTGTCTGACTAGGT |
| | | GTCCTTCTATAATATTATGGGGTGGAGGGGGGTGGTATGGAGCAAGGGGCAAGITGGG |
| | | AAGACAACCTGTAGGGCCTGCGGGGTCTATTGGGAACCAAGCTGGAGTGCAGTGGCAC |
| | | AATCTTGGCTCACTGCAATCTCCGCCTCCTGGGTTCAAGCGATTCTCCTGCCTCAGCC |
| | | TCCCGAGTTGTTGGGATTCCAGGCATGCATGACCAGGCTCAGCTAATTTTTGTTTTTT |
| | | TGGTAGAGACGGGGTTTCACCATATTGGCCAGGCTGGTCTCCAACTCCTAATCTCAGG |
| | | TGATCTACCCACCTTGGCCTCCCAAATTGCTGGGATTACAGGCGTGAACCACTGCTCC |
| | | CTTCCCTGTCCTT |
| PolyA3 | 6592 | GAATTCACTCCTCAGGTGCAGGCTGCCTATCAGAAGGTGGTGGCTGGTGTGGCCAATG |
| | | CCCTGGCTCACAAATACCACTGAGATCTTTTTCCCTCTGCCAAAAATTATGGGGACAT |
| | | CATGAAGCCCCTTGAGCATCTGACTTCTGGCTAATAAAGGAAATTTATTTTCATTGCA |
| | | ATAGTGTGTTGGAATTTTTTGTGTCTCTCACTCGGAAGGACATATGGGAGGGCAAATC |
| | | ATTTAAAACATCAGAATGAGTATTTGGTTTAGAGTTTGGCAACATATGCCCATATGCT |
| | | GGCTGCCATGAACAAAGGTTGGCTATAAAGAGGTCATCAGTATATGAAACAGCCCCCT |
| | | GCTGTCCATTCCTTATTCCATAGAAAAGCCTTGACTTGAGGTTAGATTTTTTTTATAT |
| | | TTTGTTTTGTGTTATTTTTTTCTTTAACATCCCTAAAATTTTCCTTACATGTTTTACT |
| | | AGCCAGATTTTTCCTCCTCTCCTGACTACTCCCAGTCATAGCTGTCCCTCTTCTCTTA |
| | | TGGAGATCCCTCGACCTGCAGCCCAAGCTT |

In some embodiments, the polyA signal region is provided in Table 13. In some embodiments, the genetic element comprises a polyA sequence region chosen from polyA1, polyA2, polyA3, or a functional variant thereof. In some embodiments, the genetic element comprises the polyA signal region of PolyA1 or a functional variant thereof. In some embodiments, the polyA signal region comprises the nucleotide sequence of any one of SEQ ID NOs: 6590-6592, or a sequence with at least 70%, 75%, 80%, 85%, 90%, 95%, or 99% sequence identity thereto. In some embodiments, the polyA signal region comprises the nucleotide sequence of SEQ ID NO: 6590, or a nucleotide sequence with at least 70%, 75%, 80%, 85%, 90%, 95%, or 99% sequence identity thereto.

### Genetic Element Component: Introns

In some embodiments, the genetic element comprises at least one element to enhance the expression of a transgene encoding a payload. In some embodiments, an element that enhances expression of a transgene comprises an introns or functional variant thereof. In some embodiments, the genetic element comprises an intron or functional variant thereof. In some embodiments, the genetic element comprises at least two intron regions, e.g., at least 2 intron regions, at least 3 intron regions, at least 4 intron regions, or 5 or more intron regions.

In some embodiments, the genetic element comprises an intron chosen from a MVM intron (67-97 bps), an F.IX truncated intron 1 (300 bps), an β-globin SD/immunoglobulin heavy chain splice acceptor intron (250 bps), an adenovirus splice donor/immunoglobin splice acceptor intron (500 bps), SV40 late splice donor/splice acceptor intron (19S/16S) (180 bps), or a hybrid adenovirus splice donor/IgG splice acceptor intron (230 bps). In some embodiments, the genetic element comprises a human beta-globin intron region. In some embodiments, the human beta-globin intron region comprises the nucleotide sequence of SEQ ID NO: 6580 or 6595, or a nucleotide sequence with at least 70%, 75%, 80%, 85%, 90%, 95% or 99% sequence identity to SEQ ID NO: 6580 or 6595. In some embodiments, the genetic element comprises an ie intron 1 region. In some embodiments, the ie intron 1 region comprises the nucleotide sequence of SEQ ID NO: 6578, or a nucleotide sequence with at least 70%, 75%, 80%, 85%, 90%, 95% or 99% sequence identity thereto. In some embodiments, the genetic element comprises a first human beta-globin intron region, e.g., a first human beta-globin intron region comprising SEQ ID NO: 6595, and a second human beta-globin intron region, e.g., a second human beta-globin intron region comprising SEQ ID NO: 6580. In some embodiments, an ie intron 1 region, e.g., an ie intron 1 region comprising SEQ ID NO: 6578, and a human beta-globin intron region, e.g., a human beta-globin intron region comprising SEQ ID NO: 6595.

In some embodiments, the genetic element comprises an intron region provided in Table 67. In some embodiments, the genetic element comprises an intron region chosen from any one of Intron1 to Intron5, or a functional variant thereof. In some embodiments, the genetic element comprises Intron1. In some embodiments, the genetic element comprises Intron3. In some embodiments, the genetic element comprises Intron12. In some embodiments, the genetic element comprises Intron 12 and Intron3. In some embodiments, the genetic element comprises Intron1 and Intron12. In some embodiments, the genetic element comprises an intron region of any one of SEQ ID NOs: 6578-6588, 6595, or 6607-6609, or a sequence with at least 70%, 75%, 80%, 85%, 90%, 95%, or 99% sequence identity thereto. In some embodiments, the intron region comprises the nucleotide sequence of SEQ ID NO: 6578, or a nucleotide sequence with at least 70%, 75%, 80%, 85%, 90%, 95%, or 99% sequence identity thereto. In some embodiments, the intron region comprises the nucleotide sequence of SEQ ID NO: 6580, or a nucleotide sequence with at least 70%, 75%, 80%, 85%, 90%, 95%, or 99% sequence identity thereto. In some embodiments, the intron region comprises the nucleotide sequence of SEQ ID NO: 2040A, or a nucleotide sequence with at least 70%, 75%, 80%, 85%, 90%, 95%, or 99% sequence identity thereto.

TABLE 67
Intron Sequence Regions

| | SEQ | |
|---|---|---|
| Name | ID NO | Sequence |
| Intron 1 | 6578 | GTAAGTACCGCCTATAGAGTCTATAGGCCCAC |
| Intron2 | 6579 | GTAAGTACCGCCTAT |
| Intron3 | 6580 | AAAAAATGCTTTCTTCTTTTAATATACTTTTTTGTTTATCTTATTTCTAATACTT |
| | | TCCCTAATCTCTTTCTTTCAGGGCAATAATGATACAATGTATCATGCCTCTTTGC |
| | | ACCATTCTAAAGAATAACAGTGATAATTTCTGGGTTAAGGCAATAGCAATATTTC |
| | | TGCATATAAATATTTCTGCATATAAATTGTAACTGATGTAAGAGGTTTCATATTG |
| | | CTAATAGCAGCTACAATCCAGCTACCATTCTGCTTTTATTTTATGGTTGGGATAA |
| | | GGCTGGATTATTCTGAGTCCAAGCTAGGCCCTTTTGCTAATCATGTTCATACCTC |
| | | TTATCTTCCTCCCACAG |
| Intron4 | 6581 | TCTGCATATAAATTGTAACTGATGTAAGAGGTTTCATATTGCTAATAGCAGCTAC |
| | | AATCCAGCTACCATTCTGCTTTTATTTTATGGTTGGGATAAGGCTGGATTATTCT |
| | | GAGTCCAAGCTAGGCCCTTTTGCTAATCATGTTCATACCTCTTATCTTCCTCCCA |
| | | CAG |
| Intron5 | 6582 | AAAAAATGCTTTCTTCTTTTAATATACTTTTCTTTTGCTAATCATGTTCATACCT |
| | | CTTATCTTCCTCCCACAG |
| Intron6 | 6583 | AGGCTGGATTATTCTGAGTCCAAGCTAGGCCCTTTTGCTAATCATGTTCATACCT |
| | | CTTATCTTCCTCCCACAG |
| Intron7 | 6584 | AAAAAATGCTTTCTTCTTTTCAAGCTAGGCCCTTTTGCTAATCATGTTCATACCT |
| | | CTTATCTTCCTCCCACAG |
| Intron8 | 6585 | CAAGCTAGGCCCTTTTGCTAATCATGTTCATACCTCTTATCTTCCTCCCACAG |
| Intron9 | 6586 | GAACTGAAAAACCAGAAAGTTAACTGGTAAGTTTAGTCTTTTTGTCTTTTATTTC |
| | | AGGTCCCGGATCCGGTGGTGGTGCAAATCAAAGAACTGCTCCTCAGTGGATGTTG |
| | | CCTTTACTTCTAGGCCTGTACGGAAGTGTTACTTCTGCTCTAAAAGCTGCGGAAT |
| | | TGTACCC |
| Intron10 | 6587 | GGAGTCGCTGCGCGCTGCCTTCGCCCCGTGCCCCGCTCCGCCGCCGCCTCGCGCC |
| | | GCCCGCCCCGGCTCTGACTGACCGCGTTACTCCCACAGGTGAGCGGGCGGGACGG |
| | | CCCTTCTCCTCCGGGCTGTAATTAGCGCTTGGTTTAATGACGGCTTGTTTCTTTT |
| | | CTGTGGCTGCGTGAAAGCCTTGAGGGGCTCCGGGAGGGCCCTTTGTGCGGGGGGA |
| | | GCGGCTCGGGGGGTGCGTGCGTGTGTGTGTGCGTGGGGAGCGCCGCGTGCGGCTC |
| | | CGCGCTGCCCGGCGGCTGTGAGCGCTGCGGGCGCGGCGCGGGGCTTTGTGCGCTC |
| | | CGCAGTGTGCGCGAGGGGAGCGCGGCCGGGGGCGGTGCCCCGCGGTGCGGGGGGG |
| | | GCTGCGAGGGGAACAAAGGCTGCGTGCGGGGTGTGTGCGTGGGGGGGTGAGCAGG |
| | | GGGTGTGGGCGCGTCGGTCGGGCTGCAACCCCCCCTGCACCCCCCTCCCCGAGTT |
| | | GCTGAGCACGGCCCGGCTTCGGGTGCGGGGCTCCGTACGGGGCGTGGCGCGGGGC |
| | | TCGCCGTGCCGGGCGGGGGGTGGCGGCAGGTGGGGGTGCCGGGGGGGGCGGGGCC |
| | | GCCTCGGGCCGGGGAGGGCTCGGGGGAGGGGCGCGGCGGCCCCCGGAGCGCCGGC |
| | | GGCTGTCGAGGCGCGGCGAGCCGCAGCCATTGCCTTTTATGGTAATCGTGCGAGA |
| | | GGGCGCAGGGACTTCCTTTGTCCCAAATCTGTGCGGAGCCGAAATCTGGGAGGCG |
| | | CCGCCGCACCCCCTCTAGCGGGCGCGGGGCGAAGCGGTGCGGCGCCGGCAGGAAG |
| | | GAAATGGGGGGGGAGGGCCTTCGTGCGTCGCCGCGCCGCCGTCCCCTTCTCCCTC |
| | | TCCAGCCTCGGGGCTGTCCGCGGGGGGACGGCTGCCTTCGGGGGGGACGGGGCAG |
| | | GGCGGGGTTCGGCTTCTGGCGTGTGACCGGCGGCTCTAGAGCCTCTGCTAACCAT |
| | | GTTCATGCCTTCTTCTTTTTCCTACAGCTCCTGGGCAACGTGCTGGTTATTGTGC |
| | | TGTCTCATCATTTTGGCAAAGAATTCGAG |
| Intron 11 | 6588 | CCTCTGCTAACCATGTTCATGCCTTCTTCTTTTTCCTACAG |
| Intron 12 | 6595 | TCAGATCGCCTGGAGACGCCATCCACGCTGTTTTGACCTCCATAGAAGACACCGG |
| | | GACCGATCCAGCCTCCGCGGATTCGAATCCCGGCCGGGAACGGTGCATTGGAACG |
| | | CGGATTCCCCGTGCCAAGAGTGACGTAAGTACCGCCTATAGAGTCTATAGGCCCA |
| | | CAAAAAATGCTTTCTTCTTTTAATATACTTTTTTGTTTATCTTATTTCTAATACT |
| | | TTCCCTAATCTCTTTCTTTCAGGGCAATAATGATACAATGTATCATGCCTCTTTG |
| | | CACCATTCTAAAGAATAACAGTGATAATTTCTGGGTTAAGGCAATAGCAATATTT |
| | | CTGCATATAAATATTTCTGCATATAAATTGTAACTGATGTAAGAGGTTTCATATT |
| | | GCTAATAGCAGCTACAATCCAGCTACCATTCTGCTTTTATTTTATGGTTGGGATA |
| | | AGGCTGGATTATTCTGAGTCCAAGCTAGGCCCTTTTGCTAATCATGTTCATACCT |
| | | CTTATCTTCCTCCCACAGCTCCTGGGCAACGTGCTGGTCTGTGTGCTGGCCCATC |
| | | ACTTTGGCAAAGAATT |
| Intron 13 | 6607 | ATTCGAATCCCGGCCGGGAACGGTGCATTGGAACGCGGATTCCCCGTGCCAAGAG |
| | | TGACGTAAGTACCGCCTATAGAGTCTATAGGCCCACAAAAAATGCTTTCTTCTTT |
| | | TAATATACTTTTTTGTTTATCTTATTTCTAATACTTTCCCTAATCTCTTTCTTTC |
| | | AGGGCAATAATGATACAATGTATCATGCCTCTTTGCACCATTCTAAAGAATAACA |
| | | GTGATAATTTCTGGGTTAAGGCAATAGCAATATTTCTGCATATAAATATTTCTGC |
| | | ATATAAATTGTAACTGATGTAAGAGGTTTCATATTGCTAATAGCAGCTACAATCC |
| | | AGCTACCATTCTGCTTTTATTTTATGGTTGGGATAAGGCTGGATTATTCTGAGTC |
| | | CAAGCTAGGCCCTTTTGCTAATCATGTTCATACCTCTTATCTTCCTCCCACAGCT |
| | | CCTGGGCAACGTGCTGGTCTGTGTGCTGGCCCATCACTTTGGCAAAGAATT |
| Intron 14 | 6608 | TCAGATCGCCTGGAGACGCCATCCACGCTGTTTTGACCTCCATAGAAGACACCGG |
| | | GACCGATCCAGCCTCCGCGGATTCGAATCCCGGCCGGGAACGGTGCATTGGAACG |
| | | CGGATTCCCCGTGCCAAGAGTGACGTAAGTACCGCCTATAGAGTCTATAGGCCCA |
| | | CTCTGCATATAAATTGTAACTGATGTAAGAGGTTTCATATTGCTAATAGCAGCTA |
| | | CAATCCAGCTACCATTCTGCTTTTATTTTATGGTTGGGATAAGGCTGGATTATTC |
| | | TGAGTCCAAGCTAGGCCCTTTTGCTAATCATGTTCATACCTCTTATCTTCCTCCC |
| | | ACAGCTCCTGGGCAACGTGCTGGTCTGTGTGCTGGCCCATCACTTTGGCAAAGAA |
| | | TT |
| Intron 15 | 6609 | TCAGATCGCCTGGAGACGCCATCCACGCTGTTTTGACCTCCATAGAAGACACCGG |
| | | GACCGATCCAGGTAAGTACCGCCTATAGAGTCTATAGGCCCACAAAAAATGCTTT |
| | | CTTCTTTTAATATACTTTTTTGTTTATCTTATTTCTAATACTTTCCCTAATCTCT |
| | | TTCGCTGGATTATTCTGAGTCCAAGCTAGGCCCTTTTGCTAATCATGTTCATACC |
| | | TCTTATCTTCCTCCCACAGCTCCTGGGCAACGTGCTGGTCTGTGTGCTGGCCCAT |
| | | CACTTTGGCAAAGAATT |

### Genetic Element Component: Exons

In some embodiments, the genetic element comprises an exon sequence region. In some embodiments, the genetic element comprises at least 2, at least 3, at least 4, or at least 5 exon regions. In some embodiments, the genetic element comprises 2 exon regions.

In some embodiments, the genetic element comprises an ie exon 1 region. In some embodiments, the ie exon 1 region comprises the nucleotide sequence of SEQ ID NO: 6573, or a nucleotide sequence with at least 70%, 75%, 80%, 85%, 90%, 95%, or 99% sequence identity thereto. In some embodiments, the genetic element comprises a human beta-globin exon region. In some embodiments, the human beta-globin exon region comprises a nucleotide sequence of SEQ ID NO: 6576 or a sequence with at least 70%, 75%, 80%, 85%, 90%, 95%, or 99% sequence identity thereto. Tn some embodiments, the genetic element comprises an ie exon 1 region and a human beta-globin exon region.

In some embodiments, the exon region is provided in Table 66. In some embodiments, the genetic element comprises an exon region chosen from Exon1, Exon2, Exon3, Exon4, or a function variant thereof. In some embodiments, the genetic element comprises Exon1. In some embodiments, the genetic element comprises Exon4. In some embodiments, the genetic element comprises Exon1 and Exon4. In some embodiments, the exon region comprises the nucleotide sequence of any one of SEQ ID NOs: 6573-6577, or a nucleotide sequence with at least 70%, 75%, 80%, 85%, 90%, 95%, or 99% sequence identity thereto. In some embodiments, the exon region comprises the nucleotide sequence of SEQ ID NO: 6573, or a nucleotide sequence with at least 70%, 75%, 80%, 85%, 90%, 95%, or 99% sequence identity thereto. In some embodiments, the exon region comprises the nucleotide sequence of SEQ ID NO: 6576, or a nucleotide sequence with at least 70%, 75%, 80%, 85%, 90%, 95%, or 99% sequence identity thereto.

TABLE 66
Exon Sequence Regions

| SEQ | |
|---|---|
| ID | |
| NO | Sequence |
| 6573 | TCAGATCGCCTGGAGACGCCATCCACGCTG |
| | TTTTGACCTCCATAGAAGACACCGGGACCG |
| | ATCCAGCCTCCGCGGATTCGAATCCCGGCC |
| | GGGAACGGTGCATTGGAACGCGGATTCCCC |
| | GTGCCAAGAGTGAC |
| 6574 | TTGACCTCCATAGAAGACACCGGGACCGAT |
| | CCAGCCTCCGCGGATTCGAATCCCGGCCGG |
| | GAACGGTGCATTGGAACGCGGATTCCCCGT |
| | GCCAAGAGTGAC |
| 6575 | ATTCGAATCCCGGCCGGGAACGGTGCATTG |
| | GAACGCGGATTCCCCGTGCCAAGAGTGAC |
| 6576 | CTCCTGGGCAACGTGCTGGTCTGTGTGCTG |
| | GCCCATCACTTTGGCAAAGAATT |
| 6577 | CTCCTGGGCAACGTGCTGGTTATTGTGCTG |
| | TCTCATCATTTTGGCAAAGAATTC |

### Payloads

In some embodiments, an AAV particle, e.g., an AAV particle for the vectorized delivery of an antibody molecule described herein (e.g., an anti-HER2 antibody molecule), comprises a payload. In some embodiments, an AAV particle described herein comprises at least two, at least three, or at least 4 payloads. In some embodiments, an AAV particle, e.g., an AAV particle for the vectorized delivery of an antibody molecule described herein (e.g., an anti-HER2 antibody molecule), comprises a nucleic acid comprising a transgene encoding a payload. In some embodiments, the payload comprises an antibody molecule, e.g., an anti-HER2 antibody molecule. In some embodiments, the payload comprises a secreted protein, an intracellular protein, an extracellular protein, a membrane protein, a structural protein, a functional protein, or a protein, e.g., a mammalian protein, involved in immune system regulation. In some embodiments, a nucleic acid comprises a transgene encoding an antibody molecule that binds to HER2.

In some embodiments, the nucleic acid molecule comprising the transgene encoding a payload further comprises a nucleotide sequence encoding a linker (e.g., a linker connecting a heavy chain variable region (VH) and a light chain variable region (VL) of an antibody molecule) and/or a cleavage site. In some embodiments, the nucleic acid molecule comprising the transgene encoding a payload further comprises a nucleotide sequence encoding a signal sequence.

In some embodiments, the nucleic acid encoding the payload may be constructed, e.g., organized, similar to, e.g., mirroring, the natural organization of an mRNA. In some embodiments, the nucleic acid encoding the payload may comprise coding and/or non-coding nucleotide sequences. In some embodiments, the nucleic acid encoding the payload may encode a coding and/or a non-coding RNA.

In such an embodiment, the nucleic acid comprising a transgene encoding a payload (e.g., an antibody molecule) is replicated and packaged into an AAV particle. In some embodiments, following transduction of a cell with an AAV particle comprising a payload (e.g., an antibody molecule), the cell expresses the payload. In some embodiments, the payload, e.g., antibody molecule, produced by a cell transduced by an AAV particle comprising the payload, is secreted from the cell.

### Antibody Molecules

In some embodiments, the nucleic acid comprises a transgene encoding an antibody molecule. In some embodiments, the encoded antibody molecule binds to HER2. For example, the encoded antibody molecule binds to an epitope, e.g., a linear or conformational epitope on HER2.

In some embodiments, the encoded antibody molecule comprises at least one immunoglobulin variable domain sequence. An antibody molecule may include, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The encoded antibodies of the present disclosure can be monoclonal or polyclonal. The encoded antibody can also be a human, humanized, CDR-grafted, or in vitro generated antibody. The encoded antibody can have a heavy chain constant region chosen from, e.g., IgG1, IgG2, IgG3, or IgG4. The encoded antibody can also have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005).

In some embodiments, an encoded antibody molecule of the present disclosure comprises a functional fragment or variant thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In some embodiments, the encoded antibody molecule can be single domain antibody. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

In some embodiments, the VH and VL regions of the encoded antibody molecule can be subdivided into regions of hypervariability, termed complementarity determining regions (CDR), interspersed with regions that are more conserved, termed framework regions (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

Complementarity determining region, and CDR, as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (Kabat numbering scheme), Al-Lazikani et al., (1997) JMB 273, 927-948 (Chothia numbering scheme). In some embodiments, the CDRs defined according the Chothia number scheme are also sometimes referred to as hypervariable loops.

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

In some embodiments, the antigen binding domain of the encoded antibody molecules of the present disclosure is the part of the encoded antibody molecule that comprises determinants that form an interface that binds to the HER2 protein or an epitope thereof. With respect to proteins (or protein mimetics), the antigen-binding site typically includes one or more loops (of at least four amino acids or amino acid mimics) that form an interface that binds to the HER2 protein. Typically, the antigen-binding site of an antibody molecule includes at least one or two CDRs and/or hypervariable loops, or more typically at least three, four, five or six CDRs and/or hypervariable loops.

The encoded antibody molecule can be a monoclonal antibody molecule or a polyclonal antibody molecule. In some embodiments, a monoclonal antibody or a monoclonal antibody composition refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

In some embodiments, the sequences of an antibody molecule to be included in an encoded payload described herein can be generated by recombinant libraries, e.g., generated by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In some embodiments, the sequences of an antibody molecule to be included in an encoded payload described herein can be generated from an antibody molecule that is designed using the VERSITOPE™ Antibody Generation or BIOATLA®, e.g., in US20130303399, US20130281303, WO2012009026, WO2016033331, WO2016036916, and U.S. Pat. No. 8,859,467, the contents of which are herein incorporated by reference in their entirety. In some embodiments, the sequences of an antibody molecule to be included in an encoded payload described herein can be derived from an antibody molecule that is designed and/or produced using the methods described, e.g., in WO2017189959 and WO2020223276, the contents of which are herein incorporated by reference in their entirety.

In some embodiments, the encoded antibody comprises an amino acid sequence of a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

In some embodiments, the encoded antibody comprises an amino acid sequence of an antibody in which the variable region, or a portion thereof, e.g., the CDRs, are generated in a non-human organism, e.g., a rat or mouse. Encoded antibody molecules comprising chimeric, CDR-grafted, and humanized antibodies are within the invention. Encoded antibody molecules comprising the sequences of antibodies generated in a non-human organism, e.g., a rat or mouse, and then modified, e.g., in the variable framework or constant region, to decrease antigenicity in a human are within the invention.

An effectively human protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the donor and the immunoglobulin providing the framework is called the acceptor. In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

In some embodiments, the consensus sequence refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. In some embodiments, the consensus framework refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

In some embodiments, the encoded antibodies comprise the sequences of humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In some embodiments, the encoded antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3. IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not recruit effector cells; or fix complement. In other embodiments, the antibody has reduced or no ability to bind an Fc receptor. For example, it is an isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). In some embodiments, a derivatized antibody molecule is one that has been modified. Methods of derivatization include but are not limited to the addition of a fluorescent moiety, a radionucleotide, a toxin, an enzyme or an affinity ligand such as biotin. In some embodiments, the nucleic acid sequence comprising the transgene encoding the antibody molecule further encodes a fluorescent moiety, a radionucleotide, a toxin, an enzyme or an affinity ligand such as biotin. Accordingly, the encoded antibody molecules of the invention are intended to include derivatized and otherwise modified forms of the antibodies described herein, including immunoadhesion molecules. For example, an encoded antibody molecule can be functionally linked (by genetic fusion or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag). In some embodiments, the nucleic acid sequence comprising the transgene encoding the antibody molecule further encodes a another antibody (e.g., a bispecific antibody or a diabody), a detectable agent, a cytotoxic agent, a pharmaceutical agent, and/or a protein or peptide that can mediate association of the antibody or antibody portion with another molecule (e.g., a tag, e.g., a tag described herein).

### Multispecific Antibody Molecules

In some embodiments, the encoded antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or tetraspecific antibody molecule. In some embodiments, the encoded anti-HER2 molecule is a multispecific antibody molecule.

In some embodiments, an encoded multispecific antibody molecule is an encoded bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv, or fragment thereof, have binding specificity for a first epitope and a scFv, or fragment thereof, have binding specificity for a second epitope. In some embodiment, the encoded anti-HER2 antibody is a bispecific antibody molecule.

In another aspect, the invention relates to a multispecific, e.g., bispecific (e.g., biparatopic), antibody molecule comprising one or more HER-2 antigen binding domains, such as but not limited to an anti-HER2 antibody or antibody fragment comprising one or more CDRs, VH, heavy chain, VL, and/or light chain thereof. Non-limiting anti-HER2 antibody molecule sequences are provided in Tables 3A-3C and 20.

In some embodiments, the antibody molecule encoded by a transgene described herein is an anti-HER2 bispecific antibody molecule. In some embodiments, the anti-HER2 bispecific antibody molecule is monovalent or bivalent. In some embodiments, the anti-HER2 bispecific antibody molecule is a biparatopic antibody molecule. In some embodiments, the anti-HER2 bispecific antibody molecule comprises an Fc region or a variant thereof, e.g., as provided in Table 20. In some embodiments, the Fc region has reduced, e.g., ablated, affinity for an Fc receptor, e.g., an Fc receptor described herein. In some embodiments, the reduced affinity is compared to an otherwise similar antibody with a wild type Fc region. In some embodiments, the Fc receptor comprises a mutation at one or more of (e.g., all of) positions I253 (e.g., I253A), H310 (e.g., H310A or H310Q), and/or H435 (e.g., H435A or H435Q), numbered according to the EU index as in Kabat. In some embodiments, a bispecific antibody molecule described herein comprises a variant Fc region with reduced effector function (e.g., reduced ADCC). In some embodiments, the Fc region comprises a mutation at one or more of (e.g., all of) positions L235 (e.g., L235V), F243 (e.g., F243L), R292 (e.g., R292P), Y300 (e.g., Y300L), and P396 (e.g., P396L), numbered according to the EU index as in Kabat.

In some embodiments, an anti-HER2 bispecific antibody molecule comprises a first antigen binding domain and a second antigen binding domain. In some embodiments, the first antigen binding domain binds domain I of HER2 and the second antigen binding domain that binds domain IV of HER2. In some embodiments, the first antigen binding domain that binds domain II of HER2 and the second antigen binding domain that binds domain IV of HER2. In some embodiments, the first antigen binding domain that binds domain III of HER2 and the second antigen binding domain that binds domain IV of HER2. In some embodiments, the first antigen binding domain that binds domain I of HER2 and the second antigen binding domain that binds domain II of HER2. In some embodiments, the first antigen binding domain that binds domain I of HER2 and the second antigen binding domain that binds domain III of HER2. In some embodiments, the first antigen binding domain that binds domain II of HER2 and the second antigen binding domain that binds domain III of HER2. In some embodiments, the first and/or second antigen binding domain comprise an IgG antibody, single-chain Fv (scFv), a scFv fragment, a Fab, a single-chain Fab (scFabs), a single-chain antibody, a diabody, an antibody variable domain, a VHH, a single domain antibody, and/or a nanobody.

In some embodiments, the first antigen binding domain (e.g., an scFv) is located at the N-terminus of the VL of the second binding domain (e.g., a full antibody, e.g., an IgG antibody), e.g., linked via a peptide linker. In some embodiments, a polypeptide of the bispecific antibody molecule comprises the following sequences, from N-terminal to C-terminal: VH of the first binding domain, first peptide linker (e.g., a (G4S)3 linker (SEQ ID NO: 4537)), VL of first binding domain, second peptide linker (e.g., a (G4S) linker (SEQ ID NO: 4535)), VL of the second binding domain and CL. In some embodiments, a polypeptide of the bispecific antibody molecule comprises the following sequences: from N-terminal to C-terminal: VH of the second binding domain, CH1, CH2, and CH3. In some embodiments, the bispecific antibody molecule comprises an Fc region that is mutated to have reduced binding to Fc receptor or reduced ADCC, e.g., an Fc region having the mutations L235V, F243L, R292P, Y300L, and P396L, numbered according to the EU index as in Kabat. In some embodiments, the first antigen binding fragment comprises an anti-HER2 binding domain that binds domain IV of HER2, e.g., an anti-HER2 scFv that binds domain IV of HER2, e.g., comprising an anti-HER2 scFv sequence disclosed in Tables 3A-3C. In some embodiments, the second antigen binding domain comprises an anti-HER2 binding domain that binds domain I of HER2, e.g., an anti-HER2 antibody molecule (e.g., an IgG antibody) that binds domain I of HER2, comprising an anti-HER2 CDR, VH, VL, HC, and/or LC sequence as provided in Tables 3A-3C.

In some embodiments, the first antigen binding domain (e.g., an antibody mimetic, e.g., a designed ankyrin repeat protein (DARPIN)) is located at the N-terminus of the VL of the second binding domain (e.g., a full antibody, e.g., an IgG antibody), e.g., linked via a peptide linker. In some embodiments, a polypeptide of the bispecific antibody molecule comprises the following sequences, from N-terminal to C-terminal: an antibody mimetic, e.g., a designed ankyrin repeat protein (DARPIN), a peptide linker (e.g., a (G4S)3 linker (SEQ ID NO: 4537)), VL of the second binding domain and CL. In some embodiments, a polypeptide of the bispecific antibody molecule comprises the following sequences: from N-terminal to C-terminal: VH of the second binding domain, CH1, CH2, and CH3. In some embodiments, the bispecific antibody molecule comprises an Fc region that is mutated to have reduced binding to Fc receptor or reduced ADCC, e.g., an Fc region having the mutations L235V, F243L, R292P, Y300L, and P396L, numbered according to the EU index as in Kabat. In some embodiments, the first antigen binding fragment comprises an anti-HER2 binding domain that binds domain I of HER2, e.g., an anti-HER2 antibody mimetic, e.g., DARPIN that binds domain I of HER2, e.g., comprising an anti-HER2 antibody mimetic, e.g., DARPIN, sequence disclosed in Tables 3A-3C. In some embodiments, the second antigen binding domain comprises an anti-HER2 binding domain that binds domain IV of HER2, e.g., an anti-HER2 antibody molecule (e.g., an IgG antibody) that binds domain IV of HER2, comprising an anti-HER2 CDR, VH, VL, HC, and/or LC sequence as provided in Tables 3A-3C.

In some embodiments, the first antigen binding domain (e.g., a Fyn SH3-derived binding polypeptide (e.g., a fynomer)) is located at the N-terminus of the VL of the second binding domain (e.g., a full antibody, e.g., an IgG antibody), e.g., linked via a peptide linker. In some embodiments, a polypeptide of the bispecific antibody molecule comprises the following sequences, from N-terminal to C-terminal: a Fyn SH3-derived binding polypeptide, a peptide linker (e.g., a (G4S)3 linker (SEQ ID NO: 4537)), VL of the second binding domain and CL. In some embodiments, a polypeptide of the bispecific antibody molecule comprises the following sequences: from N-terminal to C-terminal: VH of the second binding domain, CH1, CH2, and CH3. In some embodiments, the bispecific antibody molecule comprises an Fc region that is mutated to have reduced binding to Fc receptor or reduced ADCC, e.g., an Fc region having the mutations L235V, F243L, R292P, Y300L, and P396L, numbered according to the EU index as in Kabat. In some embodiments, the first antigen binding fragment comprises an anti-HER2 binding domain that binds domain I of HER2, e.g., a Fyn SH3-derived binding polypeptide that binds domain I of HER2, e.g., comprising a Fyn SH3-derived binding polypeptide sequence disclosed in Table 3A. In some embodiments, the second antigen binding domain comprises an anti-HER2 binding domain that binds domain IV of HER2, e.g., an anti-HER2 antibody molecule (e.g., an IgG antibody) that binds domain IV of HER2, comprising an anti-HER2 CDR, VH, VL, HC, and/or LC sequence as provided in Tables 3A-3C. In some embodiments, the second antigen binding domain comprises an anti-HER2 binding domain that binds domain II of HER2, e.g., an anti-HER2 antibody molecule (e.g., an IgG antibody) that binds domain II of HER2, comprising an anti-HER2 CDR, VH, VL, HC, and/or LC sequence as provided in Table 3A.

In a further aspect, the invention relates to a bispecific molecule comprising a first antigen binding site from a HER2 antibody of the invention as described herein above and a second antigen binding site with a different binding specificity, such as a binding specificity for a human effector cell, a human Fc receptor, a T cell receptor, a B cell receptor or a binding specificity for a non-overlapping epitope of HER2, e.g., a bispecific antibody wherein the first and second antigen binding sites do not cross-block each other for binding to HER2, e.g. when tested as described in Example 9.

Exemplary bispecific antibody molecules of the invention comprise (i) two antibodies, one with a specificity to HER2 and another to a second target that are conjugated together, (ii) a single antibody that has one chain or arm specific to HER2 and a second chain or arm specific to a second molecule, (iii) a single chain antibody that has specificity to HER2 and a second molecule, e.g., via two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′).sub.2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fc-region; and (x) a diabody. In one embodiment, the bispecific antibody of the present invention is a diabody.

In one embodiment, the second molecule is a cancer antigen/tumor-associated antigen such as carcinoembryonic antigen (CEA), prostate specific antigen (PSA), RAGE (renal antigen), .alpha.-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), ganglioside antigens, tyrosinase, gp75, c-Met, C-myc, Mart1., MelanA, MUM-1, MUM-2, MUM-3, HLA-B7, Ep-CAM or a cancer-associated integrin, such as a5133 integrin. In another embodiment, the second molecule is a T cell and/or NK cell antigen, such as CD3 or CD16. In another embodiment, the second molecule is an angiogenic factor or other cancer-associated growth factor, such as a vascular endothelial growth factor, a fibroblast growth factor, epidermal growth factor, angiogenin or a receptor of any of these, particularly receptors associated with cancer progression (for instance another one of the HER receptors; HER1, HER3, or HER4). In one embodiment, the second antigen-binding site binds a different, preferably non-blocking, site on HER2 than the one bound by the antibody of the invention. For example, the second molecule may be derived from, or cross-block HER2-binding of, trastuzumab, pertuzumab, F5, or C1.

In some embodiments, the sequences of the encoded antibody molecules of the present disclosure can be generated from bispecific or heterodimeric antibody molecules produced using protocols known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies. e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In some embodiments, the encoded antibody molecule is a bispecific Fynomer-antibody fusion protein, e.g., it comprises a plurality of immunoglobulin variable domains sequences with one of more Fynomers fused to any of the light- or heavy-chain termini, wherein a first immunoglobulin variable domain sequence of the plurality has binding specificity for a first epitope and the Fynomer has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, the encoded anti-HER2 molecule is a multispecific Fynomer-antibody fusion molecule. Fynomers that bind a target of interest can routinely be selected using recombinant technology as described for example, in Grbulovski et al., J. Biol. Chem. 282(5) 3196-3204 (2007) and WO 2008/022759. Methods for forming Fynomer-antibody fusion proteins are known in the art including, for example, Brack et al. (2014), Mol Cancer Ther 13(8):2030-2039, US2017/0281768, US2015/0105285A1, WO2014/170063A1, WO2015/141862A1, WO2013/135588A1, U.S. Ser. No. 10/996,226B2, U.S. Pat. No. 9,989,536B2, U.S. Pat. No. 9,689,879B2, U.S. Pat. No. 9,513,296B2, U.S. Ser. No. 10/323,095B2, and U.S. Pat. No. 9,593,314B2.

This present disclosure provides in some embodiments, a nucleic acid (e.g., an isolated nucleic acid) comprising a transgene encoding a payload comprising any of the above antibody molecules and genetic elements, AAV vectors, and AAV particles comprising the same.

### Chimeric Antigen Receptors

In some embodiments, the nucleic acid comprises a transgene encoding a payload (e.g., an antibody molecule that can be used to generate a chimeric antigen receptor (CAR) or a cell expressing a CAR (e.g., a CAR-expressing cell). The CAR may comprise i) an extracellular antigen binding domain, ii) a transmembrane domain, and iii) an intracellular signaling domain (which may comprise one or both of a primary signaling domain and a costimulatory domain). The CAR may further comprise a leader sequence and/or a hinge sequence. In specific embodiments, the CAR construct comprises a scFv domain, wherein the scFv may be preceded by an optional leader sequence, and followed by an optional hinge sequence, a transmembrane region, and an intracellular signaling domain, e.g., wherein the domains are contiguous with and in the same reading frame to form a single fusion protein.

The antigen binding domain of the CAR can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain of the CAR is an scFv antibody fragment that is humanized compared to the murine sequence of the scFv from which it is derived.

In some embodiments, a CAR can be used in the treatment of disease categories which include for example, cancer, autoimmune disorders, B-cell mediated diseases, inflammatory diseases, neuronal disorders, cardiovascular disease and circulatory disorders, or infectious diseases.

### Payload Antibodies

In some embodiments, the nucleic acid molecule comprises a transgene encoding an antibody molecule that binds to HER2. In some embodiments, the encoded HER2 antibody molecule comprises at least one antigen-binding domain, e.g., a variable region or antigen binding fragment thereof, from an antibody molecule described herein, e.g., antibody molecule chosen from Ab-HER-04. Ab-HER-05, Ab-HER-10, Ab-HER-15, Ab-HER-42, Ab-HER-43, Ab-HER-46, Ab-HER-47, Ab-HER-53, Ab-HER-57, Ab-HER-62. Ab-HER-69, Ab-HER-73, Ab-HER-75, Ab-HER-77, Ab-HER-78, Ab-HER-82, Ab-HER-88, Ab-HER-89, Ab-HER-90, Ab-HER-91, Ab-HER-92, Ab-HER-97, Ab-HER-98, Ab-HER-99. Ab-HER-100, or Ab-HER-101. e.g., as described in Tables 3A-3B, or a sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding HER2 antibody molecule comprises the nucleotide sequence of at least one antigen-binding domain, e.g., a variable region or antigen binding fragment thereof, from an antibody molecule described herein, e.g., antibody molecule chosen from Ab-HER-04, Ab-HER-05, Ab-HER-10, Ab-HER-15, Ab-HER-42, Ab-HER-43, Ab-HER-46, Ab-HER-47, Ab-HER-53, Ab-HER-57, Ab-HER-62, Ab-HER-69, Ab-HER-73, Ab-HER-75, Ab-HER-77, Ab-HER-78, Ab-HER-82, Ah-HER-88, Ah-HER-89, Ab-HER-90, Ah-1HER-91, Ab-HER-92, Ah-HER-97, Ab-HER-98, Ab-HER-99, Ab-HER-100, or Ab-HER-101, e.g., as described in Tables 3A-3C, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 molecule comprises at least one, two, three, or four variable regions from an antibody molecule described herein, e.g., an antibody molecule chosen from Table 3A, 3B, or 3C, e.g., an antibody molecule chosen from Ab-HER-04, Ab-HER-05, Ab-HER-10, Ab-HER-15, Ab-HER-42, Ab-HER-43, Ab-HER-46, Ab-HER-47, Ab-HER-53, Ab-HER-57, Ab-HER-62, Ab-HER-69. Ab-HER-73, Ab-HER-75, Ab-HER-77, Ab-HER-78, Ab-HER-82, Ab-HER-88, Ab-HER-89, Ab-HER-90, Ab-HER-91, Ab-HER-92, Ab-HER-97, Ab-HER-98, Ab-HER-99, Ab-HER-100, or Ab-HER-101, e.g., as described in Tables 3A-3C, or a sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding the anti-HER2 molecule comprises the nucleotide sequence of at least one, two, three, or four variable regions from an antibody molecule described herein, e.g., an antibody molecule chosen from Table 3A, 3B, 3C, e.g., an antibody molecule chosen from Ab-HER-04, Ab-HER-05, Ab-HER-10, Ab-HER-15, Ab-HER-42, Ab-HER-43, Ab-HER-46, Ab-HER-47, Ab-HER-53, Ab-HER-57, Ab-HER-62, Ab-HER-69, Ab-HER-73, Ab-HER-75. Ab-HER-77, Ab-HER-78, Ab-HER-82, Ab-HER-88, Ab-HER-89, Ab-HER-90, Ab-HER-91, Ab-HER-92, Ab-HER-97. Ab-HER-98, Ab-HER-99, Ab-HER-100, or Ab-HER-101, e.g., as described in Tables 3A-3C, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 molecule comprises a heavy chain variable region from an antibody molecule described herein, e.g., chosen from Ab-HER-04, Ab-HER-05, Ab-HER-10, Ab-HER-15. Ab-HER-42, Ab-HER-43, Ab-HER-46, Ab-HER-47, Ab-HER-53, Ab-HER-57, Ab-HER-62, Ab-HER-69, Ab-HER-73, Ab-HER-75, Ab-HER-77, Ab-HER-78, Ab-HER-82, Ab-HER-88. Ab-HER-89, Ab-HER-90, Ab-HER-91, Ab-HER-92, Ab-HER-97, Ab-HER-98, Ab-HER-99, Ab-HER-100, or Ab-HER-101, e.g., as described in Tables 3A-3C, or a sequence substantially identical (e.g., having at least about 70%, 75%, 80%, 85%, 90%, 92%, 95%, 97%, 98%, or 99% sequence identity) to any of the aforesaid sequences.

In some embodiments, the nucleotide sequence encoding the anti-HER2 molecule comprises the nucleotide sequence of a heavy chain variable region from an antibody molecule described herein, e.g., chosen from Ab-HER-04, Ab-HER-05, Ab-HER-10, Ab-HER-15, Ab-HER-42, Ab-HER-43, Ab-HER-46, Ab-HER-47, Ab-HER-53, Ab-HER-57, Ab-HER-62, Ab-HER-69, Ab-HER-73, Ab-HER-75. Ab-HER-77, Ab-HER-78, Ab-HER-82, Ab-HER-88, Ab-HER-89, Ab-HER-90, Ab-HER-91, Ab-HER-92, Ab-HER-97. Ab-HER-98, Ab-HER-99, Ab-HER-100, or Ab-HER-101, e.g., as described in Tables 3A-3C, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 molecule comprises a light chain variable region from an antibody molecule described herein, e.g., chosen from Ab-HER-04, Ab-HER-05, Ab-HER-10, Ab-HER-15. Ab-HER-42, Ab-HER-43, Ab-HER-46, Ab-HER-47, Ab-HER-53, Ab-HER-57, Ab-HER-62, Ab-HER-69, Ab-HER-73, Ab-HER-75, Ab-HER-77, Ab-HER-78, or Ab-HER-82, e.g., as described in Table 3A, or a sequence substantially identical (e.g., having at least about 70%, 75%, 80%, 85%, 90%, 92%, 95%, 97%, 98%, or 99% sequence identity) to any of the aforesaid sequences.

In some embodiments, the nucleic acid encoding the anti-HER2 molecule comprises the nucleotide sequence of a light chain variable region from an antibody molecule described herein, e.g., chosen from Ab-HER-04, Ab-HER-05, Ab-HER-10, Ab-HER-15, Ab-HER-42, Ab-HER-43, Ab-HER-46, Ab-HER-47, Ab-HER-53, Ab-HER-57, Ab-HER-62, Ab-HER-69, Ab-HER-73, Ab-HER-75, Ab-HER-77, Ab-HER-78, or Ab-HER-82, e.g., as described in Table 3A, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 antibody molecule comprises a heavy chain constant region, e.g., a human IgG1 constant region or a murine IgG2A. In some embodiments, the encoded heavy chain constant comprises an amino acid sequence set forth in Table 20, or a sequence substantially identical (e.g., having at least about 70%, 75%, 80%, 85%, 90%, 92%, 95%, 97%, 98%, or 99% sequence identity) to any of the aforesaid sequences. In some embodiments, nucleic acid encoding the heavy chain constant region comprises a nucleotide sequence set forth in Table 20, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 antibody molecule comprises a kappa light chain constant region, e.g., a human kappa light chain constant region or a murine kappa light chain constant region. In some embodiments, the encoded light chain constant comprises an amino acid sequence set forth in Table 20, or a sequence substantially identical (e.g., having at least about 70%, 75%, 80%, 85%, 90%, 92%, 95%, 97%, 98%, or 99% sequence identity) to any of the aforesaid sequences. In some embodiments, nucleic acid encoding the light chain constant region comprises a nucleotide sequence set forth in Table 20, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 antibody molecule comprises a heavy chain constant region of an IgG1, e.g., a human IgG1, and a kappa light chain constant region, e.g. a human kappa light chain constant region, e.g., a heavy and light chain constant region comprising an amino acid sequence set forth in Table 20, or a sequence substantially identical (e.g., having at least about 80%, 85%, 90%, 92%, 95%, 97%, 98%, or 99% sequence identity) thereto. In some embodiments, the nucleotide sequence encoding the anti-HER2 antibody comprises the nucleotide sequence of a heavy chain constant region of an IgG1 (e.g., human IgG1) and the nucleotide sequence of a kappa light chain constant region (e.g., a human kappa light chain constant region) comprising a nucleotide sequence set forth in Table 20, or a nucleotide sequence substantially identical (e.g., having at least about 80%, 85%, 90%, 92%, 95%, 97%, 98%, or 99% sequence identity) thereto.

In some embodiments, the encoded anti-HER2 antibody molecule comprises an Fc region or a variant thereof, e.g., as provided in Table 20. In some embodiments, the Fc region has reduced, e.g., ablated, affinity for an Fc receptor, e.g., an Fc receptor described herein. In some embodiments, the reduced affinity is compared to an otherwise similar antibody with a wild type Fc region. In some embodiments, the Fc receptor comprises a mutation at one or more of (e.g., all of) positions I253 (e.g., I253A), H310 (e.g., H310A or H310Q), and/or H435 (e.g., H435A or H435Q), numbered according to the EU index as in Kabat. In some embodiments, a bispecific antibody molecule described herein comprises a variant Fc region with reduced effector function (e.g., reduced ADCC). In some embodiments, the Fc region comprises a mutation at one or more of (e.g., all of) positions L235 (e.g., L235V), F243 (e.g., F243L), R292 (e.g., R292P), Y300 (e.g., Y300L), and P396 (e.g., P396L), numbered according to the EU index as in Kabat.

In some embodiments, the encoded HER2 antibody molecule comprises a heavy chain variable region and a constant region, a light chain variable region and a constant region, or both, comprising an amino acid sequence of Tables 3A-3C and Table 20; or is encoded by a nucleotide sequence of Tables 3A-3C and Table 20; or a sequence substantially identical (e.g., having at least about 80%, 85%, 90%, 92%, 95%, 97%, 98%, or 99% sequence identity) to any of the aforesaid sequences.

In some embodiments, the encoded HER2 antibody molecule comprises a heavy chain, light chain, or both a heavy chain and a light chain from an antibody molecule described herein, e.g., chosen from Ab-HER-04, Ab-HER-05, Ab-HER-10, Ab-HER-15, Ab-HER-42, Ab-HER-43, Ab-HER-46, Ab-HER-47, Ah-HER-53, Ah-HER-57, Ah-HER-62, Ab-HER-69, Ah-HER-73, Ah-HER-75, Ah-HER-77, Ab-HER-78, Ab-HER-82, Ab-HER-88, Ab-HER-89, Ab-HER-90, Ab-HER-91, Ab-HER-92, Ab-HER-97, Ab-HER-98, Ab-HER-99, Ab-HER-100, or Ab-HER-101, e.g., as described in Tables 3A-3C, or a sequence substantially identical (e.g., having at least about 70%, 75%, 80%, 85%, 90%, 92%, 95%, 97%, 98%, or 99% sequence identity) to any of the aforesaid sequences.

In some embodiments, the nucleic acid encoding HER2 antibody molecule comprises the nucleotide sequence of a heavy chain, light chain, or both a heavy chain and a light chain from an antibody molecule described herein, e.g., chosen from Ab-HER-04, Ab-HER-05, Ab-HER-10, Ab-HER-15, Ab-HER-42, Ab-HER-43, Ab-HER-46, Ab-HER-47, Ab-HER-53, Ab-HER-57, Ab-HER-62. Ab-HER-69, Ab-HER-73, Ab-HER-75, Ab-HER-77, Ab-HER-78, or Ab-HER-82, Ab-HER-88, Ab-HER-89, Ab-HER-90. Ab-HER-91, Ab-HER-92, Ab-HER-97, Ab-HER-98, Ab-HER-99, Ab-HER-100, or Ab-HER-101, e.g., as described in Tables 3A-3C, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-HER2 molecule comprises at least one, two, or three complementarity determining regions (CDRs) from a heavy chain variable region comprising an amino acid sequence in Tables 3A-3C, or is encoded by a nucleotide sequence in Tables 3A-3C; or a sequence substantially identical (e.g., having at least about 80%, 85%, 90%, 92%, 95%, 97%, 98%, or 99% sequence identity) to any of the aforesaid sequences. In some embodiments, one or more of the CDRs (or collectively all of the CDRs) have one, two, three, four, five or more changes, e.g., amino acid substitutions, insertions, or deletions, relative to the amino acid sequence shown in Tables 3A-3C, or encoded by a nucleotide sequence shown in Tables 3A-3C. In certain embodiments, the encoded anti-HER2 antibody molecule includes a substitution in a heavy chain CDR, e.g., one or more substitutions in a CDR1, CDR2 and/or CDR3 of the heavy chain. In some embodiments, the CDRs are according to the Kabat definition (e.g., as set out in Tables 3A-3C). In some embodiments, the CDRs are according to the Chothia definition (e.g., as set out in Table 3A).

In some embodiments, the encoded anti-HER2 molecule comprises at least one, two, or three complementarity determining regions (CDRs) from a light chain variable region comprising an amino acid sequence in Table 3A, or is encoded by a nucleotide sequence in Table 3A; or a sequence substantially identical (e.g., having at least about 80%, 85%, 90%, 92%, 95%, 97%, 98%, or 99% sequence identity) to any of the aforesaid sequences. In some embodiments, one or more of the CDRs (or collectively all of the CDRs) have one, two, three, four, five or more changes, e.g., amino acid substitutions, insertions, or deletions, relative to the amino acid sequence shown in Table 3A, or encoded by a nucleotide sequence shown in Table 3A. In certain embodiments, the encoded anti-HER2 antibody molecule includes a substitution in a light chain CDR, e.g., one or more substitutions in a CDR1, CDR2 and/or CDR3 of the heavy chain. In some embodiments, the CDRs are according to the Kabat definition (e.g., as set out in Tables 3A-3C). In some embodiments, the CDRs are according to the Chothia definition (e.g., as set out in Table 3A).

In some embodiments, the encoded anti-HER2 antibody molecule comprises at least one, two, three, four, five or six CDRs (or collectively all of the CDRs) from a heavy and light chain variable region comprising an amino acid sequence shown in Tables 3A-3C, or is encoded by a nucleotide sequence shown in Tables 3A-3C. In some embodiments, one or more of the CDRs (or collectively all of the CDRs) have one, two, three, four, five, six or more changes, e.g., amino acid substitutions (e.g., conservative substitutions), insertions, or deletions, relative to the CDRs shown in Tables 3A-3C, or encoded by a nucleotide sequence shown in Tables 3A-3C. In some embodiments, the CDRs are according to the Kabat definition (e.g., as set out in Tables 3A-3C). In some embodiments, the CDRs are according to the Chothia definition (e.g., as set out in Table 3A).

In some embodiments, the encoded anti-HER2 antibody molecule comprises all three CDRs from a heavy chain variable region, all three CDRs from light chain variable region, or both (e.g., all six CDRs from a heavy chain variable region and a light chain variable region) comprising an amino acid sequence shown in Tables 3A-3C, or is encoded by a nucleotide sequence shown in Tables 3A-3C. In some embodiments, the CDRs are according to the Kabat definition (e.g., as set out in Tables 3A-3C). In some embodiments, the CDRs are according to the Chothia definition (e.g., as set out in Table 3A).

In some embodiments, the encoded anti-HER2 antibody comprises a heavy chain complementary determining region 1 (HC CDR1), a HC CDR2, and a HC CDR3, wherein the HC CDR1, HC CDR2, and HC CDR3 sequences comprise the sequences of SEQ ID NO: 5037, 5038, and 5039, respectively. In some embodiments, the encoded anti-HER2 antibody comprises a LC CDR1, a LC CDR2 and an LC CDR3, wherein the LC CDR1, LC CDR2, and LC CDR3 sequences comprise the sequences of SEQ ID NO: 5043, 5044, and 5045, respectively. In some embodiments, the encoded anti-HER2 antibody comprises HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 5037, 5038, 5039, 5043, 5044, and 5045, respectively. In some embodiments, one or more of the CDRs (or collectively all of the CDRs) have one, two, three, four, five, six or more changes, e.g., amino acid substitutions (e.g., conservative substitutions), insertions, or deletions, relative to SEQ ID NO: 5043, 5044, 5045, 5037, 5038, 5039, 5043, 5044, and 5045. In some embodiments, an antibody molecule described herein has one, two, three or four changes, e.g., substitutions, relative to SEQ ID NO: 5039.

In some embodiments, the encoded anti-HER2 antibody molecule comprises a heavy chain complementary determining region 1 (HC CDR1), a HC CDR2, and a HC CDR3, wherein the HC CDR1, HC CDR2, and HC CDR3 sequences comprise the sequences of SEQ ID NO: 5179, 5180, and 5181, respectively. In some embodiments, the encoded anti-HER2 antibody molecule comprises a LC CDR1, a LC CDR2 and an LC CDR3, wherein the LC CDR1, LC CDR2, and LC CDR3 sequences comprise the sequences of SEQ ID NO: 5182, 5183, and 5184, respectively. In some embodiments, the encoded anti-HER2 antibody molecule comprises HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 5179, 5180, 5181, 5182, 5183, and 5184, respectively.

In some embodiments, the encoded anti-HER2 antibody molecule comprises a heavy chain complementary determining region 1 (HC CDR1), a HC CDR2, and a HC CDR3, wherein the HC CDR1, HC CDR2, and HC CDR3 sequences comprise the sequences of SEQ ID NO: 5053, 5054, and, 5055, respectively. In some embodiments, the encoded anti-HER2 antibody molecule comprises a LC CDR1, a LC CDR2 and an LC CDR3, wherein the LC CDR1, LC CDR2, and LC CDR3 sequences comprise the sequences of SEQ ID NO: 5059, 5060, and 5061, respectively. In some embodiments, the encoded anti-HER2 antibody molecule comprises HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 5053, 5054, 5055, 5059, 5060, and 5061, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5117, 5118, 5119 5123, 5124, and 5125, respectively. In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5120, 5121, 5122; 5126, 5127, and 5128, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5281, 5282, 5283, 5287, 5288, and 5289, respectively. In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5284, 5285, 5286, 5287, 5291, and 5292, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5281, 5282, 6510, 5287, 5288, and 5289, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5281, 5282, 6515, 5287, 5288, and 5289, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5281, 5282, 6520, 5287, 5288, and 5289, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5281, 5282, 6525, 5287, 5288, and 5289, respectively.

In some embodiments, the encoded anti-HER2 antibody molecule comprises HC CDR1, a HC CDR2, a HC CDR3, a LC CDR1, a LC CDR2 and/or an LC CDR3, wherein the HC CDR1, HC CDR2, HC CDR3, LC CDR1, LC CDR2, and LC CDR3 sequences comprise the sequences of SEQ ID NO: 5281, 5282, 6530, 5287, 5288, and 5289, respectively.

In some embodiments, the encoded anti-HER2 antibody molecule comprises a heavy chain variable region comprising the amino acid sequence of SEQ ID NO: 5001, 5367, 5172, 5106, 5010, 5069, 5192, 5224, 5090, 5110, 5254, 5258, 5130, 5262, 5270, 5326, 6511, 6516, 6521, 6526, 6531, 6536, 6539, 6542, 6545, or 6548; or encoded by the nucleotide sequence of SEQ ID NO: 5002, 5171, 5105, 5009, 5068, 5191, 5223, 5089, 5109, 5253, 5257, 5129, 5261, 5269, 5330, 6512, 6517, 6522, 6527, 6532, 6537, 6540, 6543, 6546, or 6549; or a sequence substantially identical (e.g., having at least about 80%, 85%, 90%, 92%, 95%, 97%, 98%, or 99% sequence identity) to any of the aforesaid sequences.

In some embodiments, an encoded anti-HER2 antibody molecule described herein comprises a heavy chain variable region comprising one, two, three or all of an amino acid other than D at position 102, an amino acid other than M at position 107, an amino acid other than D at position 108, and/or an amino acid other than Y at position 109, relative to a reference sequence numbered according to the amino acid sequence of SEQ ID NO: 5001. In some embodiments, the anti-HER2 antibody molecule comprises one, two, three or all of the amino acid W at position 102, the amino acid F at position 107, A at position 108, and/or L at position 109, relative to a reference sequence numbered according to the amino acid sequence of SEQ ID NO: 5001. In some embodiments, the anti-HER antibody molecule comprises the amino acid W at position 102, the amino acid F at position 107, the amino acid A position 108, and the amino acid L at position 109, relative to a reference sequence numbered according to the amino acid sequence of SEQ ID NO: 5001. In some embodiments, the anti-HER antibody molecule comprises the amino acid W at position 102, the amino acid F at position 107, and the amino acid A position 108, relative to a reference sequence numbered according to the amino acid sequence of SEQ ID NO: 5001. In some embodiments, the anti-HER antibody molecule comprises the amino acid W at position 102, the amino acid F at position 107, and the amino acid L at position 109, relative to a reference sequence numbered according to the amino acid sequence of SEQ ID NO: 5001. In some embodiments, the anti-HER antibody molecule comprises the amino acid W at position 102, the amino acid A position 108, and the amino acid L at position 109, relative to a reference sequence numbered according to the amino acid sequence of SEQ ID NO: 5001. In some embodiments, the anti-HER antibody molecule comprises the amino acid F at position 107, the amino acid A position 108, and the amino acid L at position 109, relative to a reference sequence numbered according to the amino acid sequence of SEQ ID NO: 5001.

In some embodiments, an encoded anti-HER2 antibody molecule described herein comprises a heavy chain variable region comprising an amino acid substitution at one, two, three, or all of positions 102 (e.g., D102W), 107 (e.g., M107F), 108 (e.g., D108A), and/or 109 (e.g., Y109L), relative to a reference sequence numbered according to the amino acid sequence of SEQ ID NO: 5001. In some embodiments, the anti-HER2 antibody molecule comprises the amino acid substitutions D102W, M107F, D108A, and Y109L, relative to a reference sequence numbered according to the amino acid sequence of SEQ ID NO: 5001. In some embodiments, the anti-HER2 antibody molecule comprises the amino acid substitutions D102W, M107F, and D108A, relative to a reference sequence numbered according to the amino acid sequence of SEQ ID NO: 5001. In some embodiments, the anti-HER2 antibody molecule comprises the amino acid substitutions D102W, M107F, and Y109L, relative to a reference sequence numbered according to the amino acid sequence of SEQ ID NO: 5001. In some embodiments, the anti-HER2 antibody molecule comprises the amino acid substitutions D102W, D108A. and Y109L, relative to a reference sequence numbered according to the amino acid sequence of SEQ ID NO: 5001. In some embodiments, the anti-HER2 antibody molecule comprises the amino acid substitutions M107F, D108A, and Y109L, relative to a reference sequence numbered according to the amino acid sequence of SEQ ID NO: 5001.

In some embodiments, an encoded anti-HER2 antibody described herein comprises a heavy chain variable region comprising an amino acid substitution at one, two, three, or all of positions, 98 (e.g., D98W), 100 (e.g., M100F), 101 (e.g., D101A), and/or 102 (e.g., Y102L), of the CDR3 region according to Kabat numbering (e.g., as shown in Moon et al., Mol. Cells 39(3): 217-228 (2016), which is hereby incorporated by reference in its entirety). In some embodiments, the anti-HER2 antibody molecule comprises the amino acid substitutions D98W, M100F, D101A, and Y102L, numbered according to Kabat. In some embodiments, the anti-HER2 antibody molecule comprises the amino acid substitutions D98W, M100F, and D101A, numbered according to Kabat. In some embodiments, the anti-HER2 antibody molecule comprises the amino acid substitutions D98W, M100F, and Y102L, numbered according to Kabat. In some embodiments, the anti-HER2 antibody molecule comprises the amino acid substitutions D98W, D101A, and Y102L, numbered according to Kabat. In some embodiments, the anti-HER2 antibody molecule comprises the amino acid substitutions M100F, D101A, and Y102L, numbered according to Kabat.

In some embodiments, the encoded anti-HER2 antibody molecule comprises a light chain variable region comprising the amino acid sequence of SEQ ID NO: 5006, 5176, 5020, 5014, 5086, 5196, 5228, 5094, 5114, 5256, 5260, 5134, 5266, 5274, 5339, or 5354; or encoded by the nucleotide sequence of SEQ ID NO: 5005, 5175, 5019, 5013, 5085, 5195, 5227, 5094, 5113, 5255, 5259, 5133, 5265, 5273, 5340, or 5353; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain variable region and a light chain variable region comprising the amino acid sequences of SEQ ID NO: 5110 and 5114; or encoded by the nucleotide sequences of SEQ ID NO: 5109 and 5113; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain variable region comprising the amino acid sequence of SEQ ID NO: 5110; or encoded by the nucleotide sequence of SEQ ID NO: 5109; or a sequence substantially identical (e.g., having at least about 80%, 85%, 90%, 92%, 95%, 97%, 98%, or 99% sequence identity) to any of the aforesaid sequences. In some embodiments, the encoded anti-HER2 antibody molecule comprises a light chain variable region comprising the amino acid sequence of SEQ ID NO: 5114; or encoded by the nucleotide sequence of SEQ ID NO: 5113; or a sequence substantially identical (e.g., having at least about 80%, 85%, 90%, 92%, 95%, 97%, 98%, or 99% sequence identity) to any of the aforesaid sequences. In some embodiments, the encoded anti-HER2 antibody molecule comprises a heavy chain variable region and a light chain variable region comprising the amino acid sequences of SEQ ID NO: 5110 and 5114, respectively; or encoded by the nucleotide sequences of SEQ ID NO: 5109 and 5113, respectively; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain variable region and a light chain variable region comprising the amino acid sequences of SEQ ID NO: 5270 and 5274; or encoded by the nucleotide sequences of SEQ ID NO: 5269 and 5273; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain variable region comprising the amino acid sequence of SEQ ID NO: 5270; or encoded by the nucleotide sequence of SEQ ID NO: 5269; or a sequence substantially identical (e.g., having at least about 80%, 85%, 90%, 92%, 95%, 97%, 98%, or 99% sequence identity) to any of the aforesaid sequences. In some embodiments, the encoded anti-HER2 antibody molecule comprises a light chain variable region comprising the amino acid sequence of SEQ ID NO: 5274; or encoded by the nucleotide sequence of SEQ ID NO: 5273; or a sequence substantially identical (e.g., having at least about 80%, 85%, 90%, 92%, 95%, 97%, 98%, or 99% sequence identity) to any of the aforesaid sequences. In some embodiments, the encoded anti-HER2 antibody molecule comprises a heavy chain variable region and a light chain variable region comprising the amino acid sequences of SEQ ID NO: 5270 and 5274, respectively; or encoded by the nucleotide sequences of SEQ ID NO: 5269 and 5273, respectively; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain constant region comprising the amino acid sequence of SEQ ID NO: 5004, 5018, 5012, 5026, 5030, 5212, 5214, 5216, 5220, 5264, 5278, 5280, or 5345; or encoded by the nucleotide sequence of SEQ ID NO: 5005, 5017, 50121, 5025, 5029, 5211, 5213, 5215, 5219, 5263, 5277, 5279, or 5344; or a sequence substantially identical (e.g., having at least about 80%, 85%, 90%, 92%, 95%, 97%, 98%, or 99% sequence identity) to any of the aforesaid sequences. In some embodiments, the encoded anti-HER2 antibody molecule comprises a light chain constant region comprising the amino acid sequence of SEQ ID NO: 5008, 5022, 5218, or 5222; or encoded by the nucleotide sequence of SEQ ID NO: 5007, 5021, 5218, 5221, or 5346; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain comprising the amino acid sequence of SEQ ID NO: 5034, 5174, 5050, 5066, 5084, 5194, 5226, 5092, 5112, 5254, 5258, 5132, 5264, 5272, 5331, 6513, 6518, 6523, 6528, or 6533; or encoded by the nucleotide sequence of SEQ ID NO: 5033, 5173, 5049, 5065, 5083, 5193, 5225, 5091, 5111, 5253, 5257, 5131, 5263, 5271, 5332, 6514, 6519, 6524, 6529, or 6534; or a sequence substantially identical (e.g., having at least about 80%, 85%, 90%, 92%, 95%, 97%, 98%, or 99% sequence identity) to any of the aforesaid sequences. In some embodiments, the encoded anti-HER2 antibody comprises a light chain comprising the amino acid sequence of SEQ ID NO: 5036, 5178, 5052, 5068, 5088, 5198, 5230, 5096, 5116, 5136, 5268, 5276, 5341, or 5365; or encoded by the nucleotide sequence of SEQ ID NO: 5035, 5177, 5051, 5067, 5087, 5197, 5229, 5095, 5115, 5135, 5267, 5275, 5342, or 5366; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain and a light chain comprising the amino acid sequence of SEQ ID NO: 5112 and 5116, respectively; or encoded by the nucleotide sequence of SEQ ID NO: 5111 and 5115, respectively; or a sequence substantially identical (e.g., having at least about 80%, 85%, 90%, 92%, 95%, 97%, 98%, or 99% sequence identity) to any of the aforesaid sequences.

In some embodiments, the encoded anti-HER2 antibody molecule comprises a heavy chain and a light chain comprising the amino acid sequence of SEQ ID NO: 5272 and 5276, respectively; or encoded by the nucleotide sequence of SEQ ID NO: 5271 and 5275, respectively; or a sequence substantially identical (e.g., having at least about 80%, 85%, 90%, 92%, 95%, 97%, 98%, or 99% sequence identity) to any of the aforesaid sequences.

In some embodiments, the encoded HER-2 antibody molecule comprises an anti-HER2 antigen binding domain, e.g., an scFv, and an Fc region. In some embodiments, the encoded HER-2 antibody molecule comprises an anti-HER2 scFv fused to an Fc region. In some embodiments, the encoded HER-2 antibody molecule described herein comprises an Fc region that is mutated to have reduced binding to an Fc receptor or reduced ADCC. In some embodiments, the scFv comprises the amino acid sequence of SEQ ID NO: 5349, 5352, or is encoded by the nucleotide sequence of SEQ ID NO: 5350, 5351, 5364; a sequence substantially identical (e.g., having at least about 80%, 85%, 90%, 92%, 95%, 97%, 98%, or 99% sequence identity) to any of the aforesaid sequences. In some embodiments, the Fc region comprises the amino acid sequence of SEQ ID NO: 5278, or is encoded by the nucleotide sequence of SEQ ID NO: 5277; or a sequence substantially identical (e.g., having at least about 80%, 85%, 90%, 92%, 95%, 97%, 98%, or 99% sequence identity) to any of the aforesaid sequences.

In some embodiments, the encoded HER2 antibody molecule comprises VH, VL, scFv, heavy chain, and/or light chain encoded by a codon-optimized nucleotide sequence. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI.

In some embodiments, disclosed herein is an encoded an antibody molecule that competes for binding to HER2 with the aforesaid antibody molecules. In some embodiments, disclosed herein is an encoded antibody molecule that binds to the same epitope as, substantially the same epitope as, an epitope that overlaps with, or an epitope that substantially overlaps with, the epitope of the aforesaid anti-HER2 antibody molecules.

This present disclosure provides in some embodiments, a nucleic acid (e.g., an isolated nucleic acid) comprising a transgene encoding any of the above described antibody molecules and genetic elements, AAV vectors, AAV particles, and cells comprising the same.

TABLE 3A
Exemplary Anti-HER2 Antibodies

| | SEQ | | |
|---|---|---|---|
| Ab ID | ID NO | Description | Sequence |
| Ab-HER- | 5037 | HC CDR1 | DTYIH |
| 04 | | (Kabat) | |
| | 5038 | HC CDR2 | RIYPTNGYTRYADSVKG |
| | | (Kabat) | |
| | 5039 | HC CDR3 | WGGDGFYAMDY |
| | | (Kabat) | |
| | 5040 | HC CDR1 | GFNIKDT |
| | | (Chothia) | |
| | 5041 | HC CDR2 | YPTNGY |
| | | (Chothia) | |
| | 5042 | HC CDR3 | WGGDGFYAMDY |
| | | (Chothia) | |
| | 5001 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRW |
| | | | GGDGFYAMDYWGQGTLVTVSS |
| | 5367 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRW |
| | | | GGWGFYAFALWGQGTLVTVSS |
| | 5002 | DNA VH | gaggttcagctggtggagtctggcggtggcctggtgcagccagggggctc |
| | | | actccgtttgtcctgtgcagcttctggcttcaacattaaagacacctat |
| | | | atacactgggtgcgtcaggccccgggtaagggcctggaatgggttgcaa |
| | | | ggatttatcctacgaatggttatactagatatgccgatagcgtcaaggg |
| | | | ccgtttcactataagcgcagacacatccaaaaacacagcctacctgcag |
| | | | atgaacagcctgcgtgctgaggacactgccgtctattattgttctagat |
| | | | ggggaggggacggcttctatgctatggacTACtggggtcaaggaaccct |
| | | | ggtcaccgtctcctcg |
| | 5034 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPPKSCDKTHTCPPCPAPELLGGPSVFLFPPK |
| | | | PKDTLMISRTPEVTCVVVDVSHEDPEVKFNWYVDGVEVHNAKTKPREEQY |
| | | | NSTYRVVSVLTVLHQDWLNGKEYKCKVSNKALPAPIEKTISKAKGQPREP |
| | | | QVYTLPPSRDELTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPG |
| | | | K |
| | 5033 | DNA Heavy | gaggttcagctggtggagtctggcggtggcctggtgcagccagggggctc |
| | | Chain | actccgtttgtcctgtgcagcttctggcttcaacattaaagacacctata |
| | | (hIgG1) | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaa |
| | | | agttgagcccCCCaaatcttgtgacaaaactcacacatgcccaccgtgcc |
| | | | cagcacctgaactcctggggggaccgtcagtcttcctcttccccccaaaa |
| | | | cccaaggacaccctcatgatctcccggacccctgaggtcacatgcgtggt |
| | | | ggtggacgtgagccacgaagaccctgaggtcaagttcaactggtacgtgg |
| | | | acggcgtggaggtgcataatgccaagacaaagccgcgggaggagcagtac |
| | | | aacagcacgtaccgtgtggtcagcgtcctcaccgtcctgcaccaggactg |
| | | | gctgaatggcaaggagtacaagtgcaaggtctccaacaaagccctcccag |
| | | | cccccatcgagaaaaccatctccaaagccaaagggcagccccgagaacca |
| | | | caggtgtacaccctgcccccatcccgggatgagctgaccaagaaccaggt |
| | | | cagcctgacctgcctggtcaaaggcttctatcccagcgacatcgccgtgg |
| | | | agtgggagagcaatgggcagccggagaacaactacaagaccacgcctccc |
| | | | gtgctggactccgacggctccttcttcctctacagcaagctcaccgtgga |
| | | | caagagcaggtggcagcaggggaacgtcttctcatgctccgtgatgcatg |
| | | | aggctctgcacaaccactacacgcagaagagcctctccctgtctccgggt |
| | | | aaa |
| | 5043 | LC CDR1 | RASQDVNTAVA |
| | | (Kabat) | |
| | 5044 | LC CDR2 | SASFLYS |
| | | (Kabat) | |
| | 5045 | LC CDR3 | QQHYTTPPT |
| | | (Kabat) | |
| | 5046 | LC CDR1 | RASQDVNTAVA |
| | | (Chothia) | |
| | 5047 | LC CDR2 | SASFLYS |
| | | (Chothia) | |
| | 5048 | LC CDR3 | QQHYTTPPT |
| | | (Chothia) | |
| | 5006 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5005 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5036 | Light Chain | DIQMTQSPSSLSASVGDRVTITCRASQDVNTAVAWYQQKPGKAPKLLIYS |
| | | (hIgG kappa) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5035 | DNA Light | gatatccagatgacccagtccccgagctccctgtccgcctctgtgggcga |
| | | Chain (hIgG | tagggtcaccatcacctgccgtgccagtcaggatgtgaatactgctgtag |
| | | kappa) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5179 | HC CDR1 | DYTMD |
| 05 | | (Kabat) | |
| | 5180 | HC CDR2 | DVNPNSGGSIYNQRFKG |
| | | (Kabat) | |
| | 5181 | HC CDR3 | NLGPSFYFDYW |
| | | (Kabat) | |
| | 5172 | VH | EVQLVESGGGLVQPGGSLRLSCAASGFTFTDYTMDWVRQAPGKGLEWVAD |
| | | | VNPNSGGSIYNQRFKGRFTLSVDRSKNTLYLQMNSLRAEDTAVYYCARNL |
| | | | GPSFYFDYWGQGTLVTVSS |
| | 5171 | DNA VH | gaggttcagctggtggagtctggcggtggcctggtgcagccagggggctc |
| | | | actccgtttgtcctgtgcagcttctggcttcaCcTTCaCCgacTATACCA |
| | | | TGGATtgggtgcgtcaggccccgggtaagggcctggaatgggttgcaGAC |
| | | | GTGAATcctAATTCTggtGGTTCTATAtatAATCAGCGTTTCaagggccg |
| | | | tttcactCTGagcGTTgacCGTtccaaaaacacaCTGtacctgcagatga |
| | | | acagcctgcgtgctgaggacactgccgtctattattgtGCAagaAATCTG |
| | | | gggCCTAGCttctatTTCgacTACtggggtcaaggaaccctggtcaccgt |
| | | | ctcctcg |
| | 5174 | Heavy Chain | EVQLVESGGGLVQPGGSLRLSCAASGFTFTDYTMDWVRQAPGKGLEWVAD |
| | | (hIgG1) | VNPNSGGSIYNQRFKGRFTLSVDRSKNTLYLQMNSLRAEDTAVYYCARNL |
| | | | GPSFYFDYWGQGTLVTVSSASTKGPSVFPLAPSSKSTSGGTAALGCLVKD |
| | | | YFPEPVTVSWNSGALTSGVHTFPAVLQSSGLYSLSSVVTVPSSSLGTQTY |
| | | | ICNVNHKPSNTKVDKKVEPKSCDKTHTCPPCPAPELLGGPSVFLFPPKPK |
| | | | DTLMISRTPEVTCVVVDVSHEDPEVKFNWYVDGVEVHNAKTKPREEQYNS |
| | | | TYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| | 5173 | DNA Heavy | gaggttcagctggtggagtctggcggtggcctggtgcagccagggggctc |
| | | Chain | actccgtttgtcctgtgcagcttctggcttcaCcTTCaCCgacTATACCA |
| | | (hIgG1) | TGGATtgggtgcgtcaggccccgggtaagggcctggaatgggttgcaGAC |
| | | | GTGAATcctAATTCTggtGGTTCTATAtatAATCAGCGTTTCaagggccg |
| | | | tttcactCTGagcGTTgacCGTtccaaaaacacaCTGtacctgcagatga |
| | | | acagcctgcgtgctgaggacactgccgtctattattgtGCAagaAATCTG |
| | | | gggCCTAGCttctatTTCgacTACtggggtcaaggaaccctggtcaccgt |
| | | | ctcctcggcgtcgaccaagggcccatcggtcttccccctggcaccctcct |
| | | | ccaagagcacctctgggggcacagcggccctgggctgcctggtcaaggac |
| | | | tacttccccgaacctgtgacggtctcgtggaactcaggcgccctgaccag |
| | | | cggcgtgcacaccttcccggctgtcctacagtcctcaggactctactccc |
| | | | tcagcagcgtggtgaccgtgccctccagcagcttgggcacccagacctac |
| | | | atctgcaacgtgaatcacaagcccagcaacaccaaggtggacaagaaagt |
| | | | tgagcccaaatcttgtgacaaaactcacacatgcccaccgtgcccagcac |
| | | | ctgaactcctggggggaccgtcagtcttcctcttccccccaaaacccaag |
| | | | gacaccctcatgatctcccggacccctgaggtcacatgcgtggtggtgga |
| | | | cgtgagccacgaagaccctgaggtcaagttcaactggtacgtggacggcg |
| | | | tggaggtgcataatgccaagacaaagccgcgggaggagcagtacaacagc |
| | | | acgtaccgtgtggtcagcgtcctcaccgtcctgcaccaggactggctgaa |
| | | | tggcaaggagtacaagtgcaaggtctccaacaaagccctcccagccccca |
| | | | tcgagaaaaccatctccaaagccaaagggcagccccgagaaccacaggtg |
| | | | tacaccctgcccccatcccgggaGgagAtgaccaagaaccaggtcagcct |
| | | | gacctgcctggtcaaaggcttctatcccagcgacatcgccgtggagtggg |
| | | | agagcaatgggcagccggagaacaactacaagaccacgcctcccgtgctg |
| | | | gactccgacggctccttcttcctctacagcaagctcaccgtggacaagag |
| | | | caggtggcagcaggggaacgtcttctcatgctccgtgatgcatgaggctc |
| | | | tgcacaaccactacacgcagaagagcctctccctgtctccgggtaaa |
| | 5182 | LC CDR1 | KASQDVSIGVA |
| | | (Kabat) | |
| | 5183 | LC CDR2 | SASYRYT |
| | | (Kabat) | |
| | 5184 | LC CDR3 | QQYYIYPYT |
| | | (Kabat) | |
| | 5176 | VL | DIQMTQSPSSLSASVGDRVTITCKASQDVSIGVAWYQQKPGKAPKLLIYS |
| | | | ASYRYTGVPSRFSGSGSGTDFTLTISSLQPEDFATYYCQQYYIYPYTFGQ |
| | | | GTKVEIK |
| | 5175 | DNA VL | gatatccagatgacccagtccccgagctccctgtccgcctctgtgggcga |
| | | | tagggtcaccatcacctgcAAAgccagtcaggatgtgTCCATTGGGgtag |
| | | | cctggtatcaacagaaaccaggaaaagctccgaaactactgatttactcg |
| | | | gcatccTATCGCTACACGggagtcccttctcgcttctctggatccGGTtc |
| | | | tgggacggatttcactctgaccatcagcagtctgcagccggaagacttcg |
| | | | caacttattactgtcagcaaTACtatATCTATcctTACacgttcggacag |
| | | | ggtaccaaggtggagatcaaa |
| | 5178 | Light Chain | DIQMTQSPSSLSASVGDRVTITCKASQDVSIGVAWYQQKPGKAPKLLIYS |
| | | (hIgG1) | ASYRYTGVPSRFSGSGSGTDFTLTISSLQPEDFATYYCQQYYIYPYTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5177 | DNA Light | gatatccagatgacccagtccccgagctccctgtccgcctctgtgggcga |
| | | Chain | tagggtcaccatcacctgcAAAgccagtcaggatgtgTCCATTGGGgtag |
| | | (hIgG1) | cctggtatcaacagaaaccaggaaaagctccgaaactactgatttactcg |
| | | | gcatccTATCGCTACACGggagtcccttctcgcttctctggatccGGTtc |
| | | | tgggacggatttcactctgaccatcagcagtctgcagccggaagacttcg |
| | | | caacttattactgtcagcaaTACtatATCTATcctTACacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5053 | HC CDR1 | DTYIH |
| 10 | | (Kabat) | |
| | 5054 | HC CDR2 | RIYPTNGYTRYDPKFQD |
| | | (Kabat) | |
| | 5055 | HC CDR3 | WGGDGFYAMDY |
| | | (Kabat) | |
| | 5056 | HC CDR1 | GFNIKDT |
| | | (Chothia) | |
| | 5057 | HC CDR2 | YPTNGY |
| | | (Chothia) | |
| | 5058 | HC CDR3 | WGGDGFYAMDY |
| | | (Chothia) | |
| | 5016 | VH | QVQLQQSGPELVKPGASLKLSCTASGFNIKDTYIHWVKQRPEQGLEWIGR |
| | | | IYPTNGYTRYDPKFQDKATITADTSSNTAYLQVSRLTSEDTAVYYCSRWG |
| | | | GDGFYAMDYWGQGASVTVSS |
| | 5015 | DNA VH | CAGGTGCAGCTGCAGCAGAGCGGCCCCGAGCTGGTGAAGCCCGGCGCCAG |
| | | | CCTGAAGCTGAGCTGCACCGCCAGCGGCTTCAACATCAAGGACACCTACA |
| | | | TCCACTGGGTGAAGCAGAGACCCGAGCAGGGCCTGGAGTGGATCGGCAGA |
| | | | ATCTACCCCACCAACGGCTACACCAGATACGACCCCAAGTTCCAGGACAA |
| | | | GGCCACCATCACCGCCGACACCAGCAGCAACACCGCCTACCTGCAGGTGA |
| | | | GCAGACTGACCAGCGAGGACACCGCCGTGTACTACTGCAGCAGATGGGGC |
| | | | GGCGACGGCTTCTACGCCATGGACTACTGGGGCCAGGGCGCCAGCGTGAC |
| | | | CGTGAGCAGC |
| | 5050 | Heavy Chain | QVQLQQSGPELVKPGASLKLSCTASGFNIKDTYIHWVKQRPEQGLEWIGR |
| | | (hIgG1) | IYPTNGYTRYDPKFQDKATITADTSSNTAYLQVSRLTSEDTAVYYCSRWG |
| | | | GDGFYAMDYWGQGASVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLIVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 5049 | DNA Heavy | CAGGTGCAGCTGCAGCAGAGCGGCCCCGAGCTGGTGAAGCCCGGCGCCAG |
| | | Chain | CCTGAAGCTGAGCTGCACCGCCAGCGGCTTCAACATCAAGGACACCTACA |
| | | (hIgG1) | TCCACTGGGTGAAGCAGAGACCCGAGCAGGGCCTGGAGTGGATCGGCAGA |
| | | | ATCTACCCCACCAACGGCTACACCAGATACGACCCCAAGTTCCAGGACAA |
| | | | GGCCACCATCACCGCCGACACCAGCAGCAACACCGCCTACCTGCAGGTGA |
| | | | GCAGACTGACCAGCGAGGACACCGCCGTGTACTACTGCAGCAGATGGGGC |
| | | | GGCGACGGCTTCTACGCCATGGACTACTGGGGCCAGGGCGCCAGCGTGAC |
| | | | CGTGAGCAGCgcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaG |
| | | | agttgagcccaaatcttgtgacaaaactcacacatgcccaccgtgcccag |
| | | | cacctgaactcGtggggggaccgtcagtcttcctcCtccccccaaaaccc |
| | | | aaggacaccctcatgatctcccggacccctgaggtcacatgcgtggtggt |
| | | | ggacgtgagccacgaagaccctgaggtcaagttcaactggtacgtggacg |
| | | | gcgtggaggtgcataatgccaagacaaagccgcCggaggagcagtacaac |
| | | | agcacgCTccgtgtggtcagcgtcctcaccgtcctgcaccaggactggct |
| | | | gaatggcaaggagtacaagtgcaaggtctccaacaaagccctcccagccc |
| | | | ccatcgagaaaaccatctccaaagccaaagggcagccccgagaaccacag |
| | | | gtgtacaccctgcccccatcccgggaTgagCtgaccaagaaccaggtcag |
| | | | cctgacctgcctggtcaaaggcttctatcccagcgacatcgccgtggagt |
| | | | gggagagcaatgggcagccggagaacaactacaagaccacgcctcTcgtg |
| | | | ctggactccgacggctccttcttcctctacagcaagctcaccgtggacaa |
| | | | gagcaggtggcagcaggggaacgtcttctcatgctccgtgatgcatgagg |
| | | | ctctgcacaaccactacacgcagaagagcctctccctgtctccgggtaaa |
| | 5059 | LC CDR1 | KASQDVNTAVA |
| | | (Kabat) | |
| | 5060 | LC CDR2 | SASFRYT |
| | | (Kabat) | |
| | 5061 | LC CDR3 | QQHYTTPPT |
| | | (Kabat) | |
| | 5062 | LC CDR1 | KASQDVNTAVA |
| | | (Chothia) | |
| | 5063 | LC CDR2 | SASFRYT |
| | | (Chothia) | |
| | 5064 | LC CDR3 | QQHYTTPPT |
| | | (Chothia) | |
| | 5020 | VL | DIVMTQSHKFMSTSVGDRVSITCKASQDVNTAVAWYQQKPGHSPKLLIYS |
| | | | ASFRYTGVPDRFTGSRSGTDFTFTISSVQAEDLAVYYCQQHYTTPPTFGG |
| | | | GTKVEIK |
| | 5019 | DNA VL | GACATCGTGATGACCCAGAGCCACAAGTTCATGAGCACCAGCGTGGGCGA |
| | | | CAGAGTGAGCATCACCTGCAAGGCCAGCCAGGACGTGAACACCGCCGTGG |
| | | | CCTGGTACCAGCAGAAGCCCGGCCACAGCCCCAAGCTGCTGATCTACAGC |
| | | | GCCAGCTTCAGATACACCGGCGTGCCGGACAGATTCACCGGCAGCAGAAG |
| | | | CGGCACCGACTTCACCTTCACCATCAGCAGCGTGCAGGCCGAGGACCTGG |
| | | | CCGTGTACTACTGCCAGCAGCACTACACCACCCCTCCTACCTTCGGCGGC |
| | | | GGCACCAAGGTGGAGATCAAG |
| | 5052 | Light Chain | DIVMTQSHKFMSTSVGDRVSITCKASQDVNTAVAWYQQKPGHSPKLLIYS |
| | | (hIgG1) | ASFRYTGVPDRFTGSRSGTDFTFTISSVQAEDLAVYYCQQHYTTPPTFGG |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5051 | DNA Light | GACATCGTGATGACCCAGAGCCACAAGTTCATGAGCACCAGCGTGGGCGA |
| | | Chain | CAGAGTGAGCATCACCTGCAAGGCCAGCCAGGACGTGAACACCGCCGTGG |
| | | (hIgG1) | CCTGGTACCAGCAGAAGCCCGGCCACAGCCCCAAGCTGCTGATCTACAGC |
| | | | GCCAGCTTCAGATACACCGGCGTGCCGGACAGATTCACCGGCAGCAGAAG |
| | | | CGGCACCGACTTCACCTTCACCATCAGCAGCGTGCAGGCCGAGGACCTGG |
| | | | CCGTGTACTACTGCCAGCAGCACTACACCACCCCTCCTACCTTCGGCGGC |
| | | | GGCACCAAGGTGGAGATCAAGAGAACCGTGGCCGCCCCTAGCGTGTTCAT |
| | | | CTTCCCGCCAAGCGACGAGCAGCTGAAGAGCGGCACCGCCAGCGTGGTGT |
| | | | GCCTGCTGAACAACTTCTACCCCAGAGAGGCCAAGGTGCAGTGGAAGGTG |
| | | | GACAACGCCCTGCAGAGCGGCAACAGCCAGGAGAGCGTGACCGAGCAGGA |
| | | | CAGCAAGGACAGCACCTACAGCCTGAGCAGCACCCTGACCCTGAGCAAGG |
| | | | CCGACTACGAGAAGCACAAGGTGTACGCCTGCGAGGTGACCCACCAGGGC |
| | | | CTGAGCAGCCCCGTGACCAAGAGCTTCAACAGAGGCGAGTGCt |
| Ab-HER- | 5149 | HC CDR1 | DYTMD |
| 15 | | (Kabat) | |
| | 5150 | HC CDR2 | DVNPNSGGSIYNQRFKG |
| | | (Kabat) | |
| | 5151 | HC CDR3 | NLGPSFYFDYW |
| | | (Kabat) | |
| | 5010 | VH | EVQLVESGGGLVQPGGSLRLSCAASGFTFTDYTMDWVRQAPGKGLEWVAD |
| | | | VNPNSGGSIYNQRFKGRFTLSVDRSKNTLYLQMNSLRAEDTAVYYCARNL |
| | | | GPSFYFDYWGQGTLVTVSS |
| | 5009 | DNA VH | gaggttcagctggtggagtctggcggtggcctggtgcagccagggggctc |
| | | | actccgtttgtcctgtgcagcttctggcttcaCcTTCaCCgacTATACCA |
| | | | TGGATtgggtgcgtcaggccccgggtaagggcctggaatgggttgcaGAC |
| | | | GTGAATcctAATTCTggtGGTTCTATAtatAATCAGCGTTTCaagggccg |
| | | | tttcactCTGagcGTTgacCGTtccaaaaacacaCTGtacctgcagatga |
| | | | acagcctgcgtgctgaggacactgccgtctattattgtGCAagaAATCTG |
| | | | gggCCTAGCttctatTTCgacTACtggggtcaaggaaccctggtcaccgt |
| | | | ctcctcg |
| | 5066 | Heavy Chain | EVQLVESGGGLVQPGGSLRLSCAASGFTFTDYTMDWVRQAPGKGLEWVAD |
| | | (hIgG1) | VNPNSGGSIYNQRFKGRFTLSVDRSKNTLYLQMNSLRAEDTAVYYCARNL |
| | | | GPSFYFDYWGQGTLVTVSSASTKGPSVFPLAPSSKSTSGGTAALGCLVKD |
| | | | YFPEPVTVSWNSGALTSGVHTFPAVLQSSGLYSLSSVVTVPSSSLGTQTY |
| | | | ICNVNHKPSNTKVDKKVEPKSCDKTHTCPPCPAPELLGGPSVFLFPPKPK |
| | | | DTLMISRTPEVTCVVVDVSHEDPEVKENWYVDGVEVHNAKTKPREEQYNS |
| | | | TYRVVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| | 5065 | DNA Heavy | gaggttcagctggtggagtctggcggtggcctggtgcagccagggggctc |
| | | Chain | actccgtttgtcctgtgcagcttctggcttcaCcTTCaCCgacTATACCA |
| | | (hIgG1) | TGGATtgggtgcgtcaggccccgggtaagggcctggaatgggttgcaGAC |
| | | | GTGAATcctAATTCTggtGGTTCTATAtatAATCAGCGTTTCaagggccg |
| | | | tttcactCTGagcGTTgacCGTtccaaaaacacaCTGtacctgcagatga |
| | | | acagcctgcgtgctgaggacactgccgtctattattgtGCAagaAATCTG |
| | | | gggCCTAGCttctatTTCgacTACtggggtcaaggaaccctggtcaccgt |
| | | | ctcctcggcgtcgaccaagggcccatcggtcttccccctggcaccctcct |
| | | | ccaagagcacctctgggggcacagcggccctgggctgcctggtcaaggac |
| | | | tacttccccgaacctgtgacggtctcgtggaactcaggcgccctgaccag |
| | | | cggcgtgcacaccttcccggctgtcctacagtcctcaggactctactccc |
| | | | tcagcagcgtggtgaccgtgccctccagcagcttgggcacccagacctac |
| | | | atctgcaacgtgaatcacaagcccagcaacaccaaggtggacaagaaagt |
| | | | tgagcccaaatcttgtgacaaaactcacacatgcccaccgtgcccagcac |
| | | | ctgaactcctggggggaccgtcagtcttcctcttccccccaaaacccaag |
| | | | gacaccctcatgatctcccggacccctgaggtcacatgcgtggtggtgga |
| | | | cgtgagccacgaagaccctgaggtcaagttcaactggtacgtggacggcg |
| | | | tggaggtgcataatgccaagacaaagccgcgggaggagcagtacaacagc |
| | | | acgtaccgtgtggtcagcgtcctcaccgtcctgcaccaggactggctgaa |
| | | | tggcaaggagtacaagtgcaaggtctccaacaaagccctcccagccccca |
| | | | tcgagaaaaccatctccaaagccaaagggcagccccgagaaccacaggtg |
| | | | tacaccctgcccccatcccgggaGgagAtgaccaagaaccaggtcagcct |
| | | | gacctgcctggtcaaaggcttctatcccagcgacatcgccgtggagtggg |
| | | | agagcaatgggcagccggagaacaactacaagaccacgcctcccgtgctg |
| | | | gactccgacggctccttcttcctctacagcaagctcaccgtggacaagag |
| | | | caggtggcagcaggggaacgtcttctcatgctccgtgatgcatgaggctc |
| | | | tgcacaaccactacacgcagaagagcctctccctgtctccgggtaaa |
| | 5156 | C12 Fynomer | GVTLFVALYDYTSYNTRDLSFHKGEKFQILRMEDGVWWEARSLTTGETGY |
| | | | IPSNYVAPVDSIQ |
| | 5155 | DNA C12 | GGCGTGACCCTGTTCGTGGCCCTGTACGACTACACCAGCTACAACACCAG |
| | | Fynomer | GGACCTGAGCTTCCACAAGGGCGAGAAGTTCCAGATCCTGAGGATGGAGG |
| | | | ACGGCGTGTGGTGGGAGGCCAGGAGCCTGACCACCGGCGAGACCGGCTAC |
| | | | ATCCCCAGCAACTACGTGGCCCCCGTGGACAGCATCCAG |
| | 5152 | LC CDR1 | KASQDVSIGVA |
| | | (Kabat) | |
| | 5153 | LC CDR2 | SASYRYT |
| | | (Kabat) | |
| | 5154 | LC CDR3 | QQYYIYPYT |
| | | (Kabat) | |
| | 5014 | VL | DIQMTQSPSSLSASVGDRVTITCKASQDVSIGVAWYQQKPGKAPKLLIYS |
| | | | ASYRYTGVPSRFSGSGSGTDFTLTISSLQPEDFATYYCQQYYIYPYTFGQ |
| | | | GTKVEIK |
| | 5013 | DNA VL | gatatccagatgacccagtccccgagctccctgtccgcctctgtgggcga |
| | | | tagggtcaccatcacctgcAAAgccagtcaggatgtgTCCATTGGGgtag |
| | | | cctggtatcaacagaaaccaggaaaagctccgaaactactgatttactcg |
| | | | gcatccTATCGCTACACGggagtcccttctcgcttctctggatccGGTtc |
| | | | tgggacggatttcactctgaccatcagcagtctgcagccggaagacttcg |
| | | | caacttattactgtcagcaaTACtatATCTATcctTACacgttcggacag |
| | | | ggtaccaaggtggagatcaaa |
| | 5068 | Light Chain | DIQMTQSPSSLSASVGDRVTITCKASQDVSIGVAWYQQKPGKAPKLLIYS |
| | | (hIgG1) | ASYRYTGVPSRFSGSGSGTDFTLTISSLQPEDFATYYCQQYYIYPYTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5067 | DNA Light | gatatccagatgacccagtccccgagctccctgtccgcctctgtgggcga |
| | | Chain | tagggtcaccatcacctgcAAAgccagtcaggatgtgTCCATTGGGgtag |
| | | (hIgG1) | cctggtatcaacagaaaccaggaaaagctccgaaactactgatttactcg |
| | | | gcatccTATCGCTACACGggagtcccttctcgcttctctggatccGGTtc |
| | | | tgggacggatttcactctgaccatcagcagtctgcagccggaagacttcg |
| | | | caacttattactgtcagcaaTACtatATCTATcctTACacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5071 | HC CDR1 | DTYIH |
| 42 | | (Kabat) | |
| | 5072 | HC CDR2 | RIYPTNGYTRYADSVKG |
| | | (Kabat) | |
| | 5073 | HC CDR3 | WGGDGFYAMDY |
| | | (Kabat) | |
| | 5074 | HC CDR1 | GFNIKDT |
| | | (Chothia) | |
| | 5075 | HC CDR2 | YPTNGY |
| | | (Chothia) | |
| | 5076 | HC CDR3 | WGGDGFYAMDY |
| | | (Chothia) | |
| | 5069 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5070 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5084 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPPKSCDKTHTCPPCPAPELLGGPSVFLFPPK |
| | | | PKDTLMISRTPEVTCVVVDVSHEDPEVKFNWYVDGVEVHNAKTKPREEQY |
| | | | NSTYRVVSVLTVLHQDWLNGKEYKCKVSNKALPAPIEKTISKAKGQPREP |
| | | | QVYTLPPSRDELTKNQVSLTCLVKGFYPSDIAVEWESNGQPENNYKTTPP |
| | | | VLDSDGSFFLYSKLTVDKSRWQQGNVFSCSVMHEALHNQYTQKSLSLSPG |
| | | | K |
| | 5083 | DNA Heavy | gaggttcagctggtggagtctggcggtggcctggtgcagccagggggctc |
| | | Chain | actccgtttgtcctgtgcagcttctggcttcaacattaaagacacctata |
| | | (hIgG1) | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaa |
| | | | agttgagcccCCCaaatcttgtgacaaaactcacacatgcccaccgtgcc |
| | | | cagcacctgaactcctggggggaccgtcagtcttcctcttccccccaaaa |
| | | | cccaaggacaccctcatgatctcccggacccctgaggtcacatgcgtggt |
| | | | ggtggacgtgagccacgaagaccctgaggtcaagttcaactggtacgtgg |
| | | | acggcgtggaggtgcataatgccaagacaaagccgcgggaggagcagtac |
| | | | aacagcacgtaccgtgtggtcagcgtcctcaccgtcctgcaccaggactg |
| | | | gctgaatggcaaggagtacaagtgcaaggtctccaacaaagccctcccag |
| | | | cccccatcgagaaaaccatctccaaagccaaagggcagccccgagaacca |
| | | | caggtgtacaccctgcccccatcccgggatgagctgaccaagaaccaggt |
| | | | cagcctgacctgcctggtcaaaggcttctatcccagcgacatcgccgtgg |
| | | | agtgggagagcaatgggcagccggagaacaactacaagaccacgcctccc |
| | | | gtgctggactccgacggctccttcttcctctacagcaagctcaccgtgga |
| | | | caagagcaggtggcagcaggggaacgtcttctcatgctccgtgatgcatg |
| | | | aggctctgcacaacCAGtacacgcagaagagcctctccctgtctccgggt |
| | | | aaa |
| | 5077 | LC CDR1 | RASQDVNTAVA |
| | | (Kabat) | |
| | 5078 | LC CDR2 | SASFLYS |
| | | (Kabat) | |
| | 5079 | LC CDR3 | QQHYTTPPT |
| | | (Kabat) | |
| | 5080 | LC CDR1 | RASQDVNTAVA |
| | | (Chothia) | |
| | 5081 | LC CDR2 | SASFLYS |
| | | (Chothia) | |
| | 5082 | LC CDR3 | QQHYTTPPT |
| | | (Chothia) | |
| | 5086 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5085 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5088 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5087 | DNA Light | gatatccagatgacccagtccccgagctccctgtccgcctctgtgggcga |
| | | Chain | tagggtcaccatcacctgccgtgccagtcaggatgtgaatactgctgtag |
| | | (hIgG1) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5199 | HC CDR1 | DTYIH |
| 43 | | (Kabat) | |
| | 5200 | HC CDR2 | RIYPTNGYTRYADSVKG |
| | | (Kabat) | |
| | 5201 | HC CDR3 | WGGDGFYAMDY |
| | | (Kabat) | |
| | 5202 | HC CDR1 | GFNIKDT |
| | | (Chothia) | |
| | 5203 | HC CDR2 | YPTNGY |
| | | (Chothia) | |
| | 5204 | HC CDR3 | WGGDGFYAMDY |
| | | (Chothia) | |
| | 5192 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5191 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5194 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPPKSCDKTHTCPPCPAPELLGGPSVFLFPPK |
| | | | PKDTLMASRTPEVTCVVVDVSHEDPEVKFNWYVDGVEVHNAKTKPREEQY |
| | | | NSTYRVVSVLTVLAQDWLNGKEYKCKVSNKALPAPIEKTISKAKGQPREP |
| | | | QVYTLPPSRDELTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPG |
| | | | K |
| | 5193 | DNA Heavy | gaggttcagctggtggagtctggcggtggcctggtgcagccagggggctc |
| | | Chain | actccgtttgtcctgtgcagcttctggcttcaacattaaagacacctata |
| | | (hIgG1) | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaa |
| | | | agttgagcccCCCaaatcttgtgacaaaactcacacatgcccaccgtgcc |
| | | | cagcacctgaactcctggggggaccgtcagtcttcctcttccccccaaaa |
| | | | cccaaggacaccctcatgGCAtcccggacccctgaggtcacatgcgtggt |
| | | | ggtggacgtgagccacgaagaccctgaggtcaagttcaactggtacgtgg |
| | | | acggcgtggaggtgcataatgccaagacaaagccgcgggaggagcagtac |
| | | | aacagcacgtaccgtgtggtcagcgtcctcaccgtcctgGCCcaggactg |
| | | | gctgaatggcaaggagtacaagtgcaaggtctccaacaaagccctcccag |
| | | | cccccatcgagaaaaccatctccaaagccaaagggcagccccgagaacca |
| | | | caggtgtacaccctgcccccatcccgggatgagctgaccaagaaccaggt |
| | | | cagcctgacctgcctggtcaaaggcttctatcccagcgacatcgccgtgg |
| | | | agtgggagagcaatgggcagccggagaacaactacaagaccacgcctccc |
| | | | gtgctggactccgacggctccttcttcctctacagcaagctcaccgtgga |
| | | | caagagcaggtggcagcaggggaacgtcttctcatgctccgtgatgcatg |
| | | | aggctctgcacaaccactacacgcagaagagcctctccctgtctccgggt |
| | | | aaa |
| | 5205 | LC CDR1 | RASQDVNTAVA |
| | | (Kabat) | |
| | 5206 | LC CDR2 | SASFLYS |
| | | (Kabat) | |
| | 5207 | LC CDR3 | QQHYTTPPT |
| | | (Kabat) | |
| | 5208 | LC CDR1 | RASQDVNTAVA |
| | | (Chothia) | |
| | 5209 | LC CDR2 | SASFLYS |
| | | (Chothia) | |
| | 5210 | LC CDR3 | QQHYTTPPT |
| | | (Chothia) | |
| | 5196 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5195 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5198 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5197 | DNA Light | gatatccagatgacccagtccccgagctccctgtccgcctctgtgggcga |
| | | Chain | tagggtcaccatcacctgccgtgccagtcaggatgtgaatactgctgtag |
| | | (hIgG1) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5231 | HC CDR1 | DTYIH |
| 46 | | (Kabat) | |
| | 5232 | HC CDR2 | RIYPTNGYTRYADSVKG |
| | | (Kabat) | |
| | 5233 | HC CDR3 | WGGDGFYAMDY |
| | | (Kabat) | |
| | 5234 | HC CDR1 | GFNIKDT |
| | | (Chothia) | |
| | 5235 | HC CDR2 | YPTNGY |
| | | (Chothia) | |
| | 5236 | HC CDR3 | WGGDGFYAMDY |
| | | (Chothia) | |
| | 5224 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5223 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5226 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPPKSCDKTHTCPPCPAPELLGGPSVFLFPPK |
| | | | PKDTLMASRTPEVTCVVVDVSHEDPEVKFNWYVDGVEVHNAKTKPREEQY |
| | | | NSTYRVVSVLTVLHQDWLNGKEYKCKVSNKALPAPIEKTISKAKGQPREP |
| | | | QVYTLPPSRDELTKNQVSLTCLVKGFYPSDIAVEWESNGQPENNYKTTPP |
| | | | VLDSDGSFFLYSKLTVDKSRWQQGNVFSCSVMHEALHNQYTQKSLSLSPG |
| | | | K |
| | 5225 | DNA Heavy | gaggttcagctggtggagtctggcggtggcctggtgcagccagggggctc |
| | | Chain | actccgtttgtcctgtgcagcttctggcttcaacattaaagacacctata |
| | | (hIgG1) | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaa |
| | | | agttgagcccCCCaaatcttgtgacaaaactcacacatgcccaccgtgcc |
| | | | cagcacctgaactcctggggggaccgtcagtcttcctcttccccccaaaa |
| | | | cccaaggacaccctcatgGCAtcccggacccctgaggtcacatgcgtggt |
| | | | ggtggacgtgagccacgaagaccctgaggtcaagttcaactggtacgtgg |
| | | | acggcgtggaggtgcataatgccaagacaaagccgcgggaggagcagtac |
| | | | aacagcacgtaccgtgtggtcagcgtcctcaccgtcctgcaccaggactg |
| | | | gctgaatggcaaggagtacaagtgcaaggtctccaacaaagccctcccag |
| | | | cccccatcgagaaaaccatctccaaagccaaagggcagccccgagaacca |
| | | | caggtgtacaccctgcccccatcccgggatgagctgaccaagaaccaggt |
| | | | cagcctgacctgcctggtcaaaggcttctatcccagcgacatcgccgtgg |
| | | | agtgggagagcaatgggcagccggagaacaactacaagaccacgcctccc |
| | | | gtgctggactccgacggctccttcttcctctacagcaagctcaccgtgga |
| | | | caagagcaggtggcagcaggggaacgtcttctcatgctccgtgatgcatg |
| | | | aggctctgcacaacCAGtacacgcagaagagcctctccctgtctccgggt |
| | | | aaa |
| | 5237 | LC CDR1 | RASQDVNTAVA |
| | | (Kabat) | |
| | 5238 | LC CDR2 | SASFLYS |
| | | (Kabat) | |
| | 5239 | LC CDR3 | QQHYTTPPT |
| | | (Kabat) | |
| | 5240 | LC CDR1 | RASQDVNTAVA |
| | | (Chothia) | |
| | 5241 | LC CDR2 | SASFLYS |
| | | (Chothia) | |
| | 5242 | LC CDR3 | QQHYTTPPT |
| | | (Chothia) | |
| | 5228 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5227 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5230 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5229 | DNA Light | gatatccagatgacccagtccccgagctccctgtccgcctctgtgggcga |
| | | Chain | tagggtcaccatcacctgccgtgccagtcaggatgtgaatactgctgtag |
| | | (hIgG1) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5097 | HC CDR1 | DTYIH |
| 47 | | (Kabat) | |
| | 5098 | HC CDR2 | RIYPTNGYTRYADSVKG |
| | | (Kabat) | |
| | 5099 | HC CDR3 | WGGDGFYAMDY |
| | | (Kabat) | |
| | 5100 | HC CDR1 | GFNIKDT |
| | | (Chothia) | |
| | 5101 | HC CDR2 | YPTNGY |
| | | (Chothia) | |
| | 5102 | HC CDR3 | WGGDGFYAMDY |
| | | (Chothia) | |
| | 5090 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5089 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5092 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPPKSCDKTHTCPPCPAPELLGGPSVFLFPPK |
| | | | PKDTLMASRTPEVTCVVVDVSHEDPEVKFNWYVDGVEVHNAKTKPREEQY |
| | | | NSTYRVVSVLTVLAQDWLNGKEYKCKVSNKALPAPIEKTISKAKGQPREP |
| | | | QVYTLPPSRDELTKNQVSLTCLVKGFYPSDIAVEWESNGQPENNYKTTPP |
| | | | VLDSDGSFFLYSKLTVDKSRWQQGNVFSCSVMHEALHNAYTQKSLSLSPG |
| | | | K |
| | 5091 | DNA Heavy | gaggttcagctggtggagtctggcggtggcctggtgcagccagggggctc |
| | | Chain | actccgtttgtcctgtgcagcttctggcttcaacattaaagacacctata |
| | | (hIgG1) | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaa |
| | | | agttgagcccCCCaaatcttgtgacaaaactcacacatgcccaccgtgcc |
| | | | cagcacctgaactcctggggggaccgtcagtcttcctcttccccccaaaa |
| | | | cccaaggacaccctcatgGCAtcccggacccctgaggtcacatgcgtggt |
| | | | ggtggacgtgagccacgaagaccctgaggtcaagttcaactggtacgtgg |
| | | | acggcgtggaggtgcataatgccaagacaaagccgcgggaggagcagtac |
| | | | aacagcacgtaccgtgtggtcagcgtcctcaccgtcctgGCCcaggactg |
| | | | gctgaatggcaaggagtacaagtgcaaggtctccaacaaagccctcccag |
| | | | cccccatcgagaaaaccatctccaaagccaaagggcagccccgagaacca |
| | | | caggtgtacaccctgcccccatcccgggatgagctgaccaagaaccaggt |
| | | | cagcctgacctgcctggtcaaaggcttctatcccagcgacatcgccgtgg |
| | | | agtgggagagcaatgggcagccggagaacaactacaagaccacgcctccc |
| | | | gtgctggactccgacggctccttcttcctctacagcaagctcaccgtgga |
| | | | caagagcaggtggcagcaggggaacgtcttctcatgctccgtgatgcatg |
| | | | aggctctgcacaacGCTtacacgcagaagagcctctccctgtctccgggt |
| | | | aaa |
| | 5103 | LC CDR1 | RASQDVNTAVA |
| | | (Kabat) | |
| | 5104 | LC CDR2 | SASFLYS |
| | | (Kabat) | |
| | 5105 | LC CDR3 | QQHYTTPPT |
| | | (Kabat) | |
| | 5106 | LC CDR1 | RASQDVNTAVA |
| | | (Chothia) | |
| | 5107 | LC CDR2 | SASFLYS |
| | | (Chothia) | |
| | 5108 | LC CDR3 | QQHYTTPPT |
| | | (Chothia) | |
| | 5094 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5093 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5096 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5095 | DNA Light | gatatccagatgacccagtccccgagctccctgtccgcctctgtgggcga |
| | | Chain | tagggtcaccatcacctgccgtgccagtcaggatgtgaatactgctgtag |
| | | (hIgG1) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5117 | HC CDR1 | DTYIH |
| 53 | | (Kabat) | |
| | 5118 | HC CDR2 | RIYPTNGYTRYADSVKG |
| | | (Kabat) | |
| | 5119 | HC CDR3 | WGGDGFYAMDY |
| | | (Kabat) | |
| | 5120 | HC CDR1 | GFNIKDT |
| | | (Chothia) | |
| | 5121 | HC CDR2 | YPTNGY |
| | | (Chothia) | |
| | 5122 | HC CDR3 | WGGDGFYAMDY |
| | | (Chothia) | |
| | 5110 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5109 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5112 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 5111 | DNA Heavy | gaggttcagctggtggagtctggcggtggcctggtgcagccagggggctc |
| | | Chain | actccgtttgtcctgtgcagcttctggcttcaacattaaagacacctata |
| | | (hIgG1) | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaG |
| | | | agttgagcccaaatcttgtgacaaaactcacacatgcccaccgtgcccag |
| | | | cacctgaactcGtggggggaccgtcagtcttcctcCtccccccaaaaccc |
| | | | aaggacaccctcatgatctcccggacccctgaggtcacatgcgtggtggt |
| | | | ggacgtgagccacgaagaccctgaggtcaagttcaactggtacgtggacg |
| | | | gcgtggaggtgcataatgccaagacaaagccgcCggaggagcagtacaac |
| | | | agcacgCTccgtgtggtcagcgtcctcaccgtcctgcaccaggactggct |
| | | | gaatggcaaggagtacaagtgcaaggtctccaacaaagccctcccagccc |
| | | | ccatcgagaaaaccatctccaaagccaaagggcagccccgagaaccacag |
| | | | gtgtacaccctgcccccatcccgggaTgagCtgaccaagaaccaggtcag |
| | | | cctgacctgcctggtcaaaggcttctatcccagcgacatcgccgtggagt |
| | | | gggagagcaatgggcagccggagaacaactacaagaccacgcctcTcgtg |
| | | | ctggactccgacggctccttcttcctctacagcaagctcaccgtggacaa |
| | | | gagcaggtggcagcaggggaacgtcttctcatgctccgtgatgcatgagg |
| | | | ctctgcacaaccactacacgcagaagagcctctccctgtctccgggtaaa |
| | 5123 | LC CDR1 | RASQDVNTAVA |
| | | (Kabat) | |
| | 5124 | LC CDR2 | SASFLYS |
| | | (Kabat) | |
| | 5125 | LC CDR3 | QQHYTTPPT |
| | | (Kabat) | |
| | 5126 | LC CDR1 | RASQDVNTAVA |
| | | (Chothia) | |
| | 5127 | LC CDR2 | SASFLYS |
| | | (Chothia) | |
| | 5128 | LC CDR3 | QQHYTTPPT |
| | | (Chothia) | |
| | 5114 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5113 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5116 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5115 | DNA Light | gatatccagatgacccagtccccgagctccctgtccgcctctgtgggcga |
| | | Chain | tagggtcaccatcacctgccgtgccagtcaggatgtgaatactgctgtag |
| | | (hIgG1) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| Ab-HER- | 5293 | HC CDR1 | DTYIH |
| 57 | | Kabat) | |
| | 5294 | HC CDR2 | RIYPTNGYTRYADSVKG |
| | | (Kabat) | |
| | 5295 | HC CDR3 | WGGDGFYAMDY |
| | | (Kabat) | |
| | 5296 | HC CDR1 | GFNIKDT |
| | | (Chothia) | |
| | 5297 | HC CDR2 | YPTNGY |
| | | (Chothia) | |
| | 5298 | HC CDR3 | WGGDGFYAMDY |
| | | (Chothia) | |
| | 5254 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5253 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5299 | LC CDR1 | RASQDVNTAVA |
| | | (Kabat) | |
| | 5300 | LC CDR2 | SASFLYS |
| | | (Kabat) | |
| | 5301 | LC CDR3 | QQHYTTPPT |
| | | (Kabat) | |
| | 5302 | LC CDR1 | RASQDVNTAVA |
| | | (Chothia) | |
| | 5303 | LC CDR2 | SASFLYS |
| | | (Chothia) | |
| | 5304 | LC CDR3 | QQHYTTPPT |
| | | (Chothia) | |
| | 5256 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5255 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5351 | scFv | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSSGGGGSGGGGSGGGGSDIQMTQSPSSLSASV |
| | | | GDRVTITCRASQDVNTAVAWYQQKPGKAPKLLIYSASFLYSGVPSRFSGS |
| | | | RSGTDFTLTISSLQPEDFATYYCQQHYTTPPTFGQGTKVEIK |
| | 5352 | scFv DNA | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcgggtggtggtggatccggaggaggaggaAGTggaggtggag |
| | | | gtTCTgatatccagatgacccagtccccgagctccctgtccgcctctgtg |
| | | | ggcgatagggtcaccatcacctgccgtgccagtcaggatgtgaatactgc |
| | | | tgtagcctggtatcaacagaaaccaggaaaagctccgaaactactgattt |
| | | | actcggcatccttcctcTACtctggagtcccttctcgcttctctggatcc |
| | | | agatctgggacggatttcactctgaccatcagcagtctgcagccggaaga |
| | | | cttcgcaacttattactgtcagcaacattatactactcctcccacgttcg |
| | | | gacagggtaccaaggtggagatcaaa |
| Ab-HER- | 5305 | HC CDR1 | DYTMD |
| 62 | | (Kabat) | |
| | 5306 | HC CDR2 | DVNPNSGGSIYNQRFKG |
| | | (Kabat) | |
| | 5307 | HC CDR3 | NLGPSFYFDYW |
| | | (Kabat) | |
| | 5258 | VH | EVQLVESGGGLVQPGGSLRLSCAASGFTFTDYTMDWVRQAPGKGLEWVAD |
| | | | VNPNSGGSIYNQRFKGRFTLSVDRSKNTLYLQMNSLRAEDTAVYYCARNL |
| | | | GPSFYFDYWGQGTLVTVSS |
| | 5257 | DNA VH | gaggttcagctggtggagtctggcggtggcctggtgcagccagggggctc |
| | | | actccgtttgtcctgtgcagcttctggcttcaCcTTCaCCgacTATACCA |
| | | | TGGATtgggtgcgtcaggccccgggtaagggcctggaatgggttgcaGAC |
| | | | GTGAATcctAATTCTggtGGTTCTATAtatAATCAGCGTTTCaagggccg |
| | | | tttcactCTGagcGTTgacCGTtccaaaaacacaCTGtacctgcagatga |
| | | | acagcctgcgtgctgaggacactgccgtctattattgtGCAagaAATCTG |
| | | | gggCCTAGCttctatTTCgacTACtggggtcaaggaaccctggtcaccgt |
| | | | ctcctcg |
| | 5308 | LC CDR1 | KASQDVSIGVA |
| | | (Kabat) | |
| | 5309 | LC CDR2 | SASYRYT |
| | | (Kabat) | |
| | 5310 | LC CDR3 | QQYYIYPYT |
| | | (Kabat) | |
| | 5260 | VL | DIQMTQSPSSLSASVGDRVTITCKASQDVSIGVAWYQQKPGKAPKLLIYS |
| | | | ASYRYTGVPSRFSGSGSGTDFTLTISSLQPEDFATYYCQQYYIYPYTFGQ |
| | | | GTKVEIK |
| | 5259 | DNA VL | gatatccagatgacccagtccccgagctccctgtccgcctctgtgggcga |
| | | | tagggtcaccatcacctgcAAAgccagtcaggatgtgTCCATTGGGgtag |
| | | | cctggtatcaacagaaaccaggaaaagctccgaaactactgatttactcg |
| | | | gcatccTATCGCTACACGggagtcccttctcgcttctctggatccGGTtc |
| | | | tgggacggatttcactctgaccatcagcagtctgcagccggaagacttcg |
| | | | caacttattactgtcagcaaTACtatATCTATcctTACacgttcggacag |
| | | | ggtaccaaggtggagatcaaa |
| | 5349 | scFv | EVQLVESGGGLVQPGGSLRLSCAASGFTFTDYTMDWVRQAPGKGLEWVAD |
| | | | VNPNSGGSIYNQRFKGRFTLSVDRSKNTLYLQMNSLRAEDTAVYYCARNL |
| | | | GPSFYFDYWGQGTLVTVSSGGGGSGGGGSGGGGSDIQMTQSPSSLSASVG |
| | | | DRVTITCKASQDVSIGVAWYQQKPGKAPKLLIYSASYRYTGVPSRFSGSG |
| | | | SGTDFTLTISSLQPEDFATYYCQQYYIYPYTFGQGTKVEIK |
| | 5350 | scFv DNA | gaggttcagctggtggagtctggcggtggcctggtgcagccagggggctc |
| | | | actccgtttgtcctgtgcagcttctggcttcaCcTTCaCCgacTATACCA |
| | | | TGGATtgggtgcgtcaggccccgggtaagggcctggaatgggttgcaGAC |
| | | | GTGAATcctAATTCTggtGGTTCTATAtatAATCAGCGTTTCaagggccg |
| | | | tttcactCTGagcGTTgacCGTtccaaaaacacaCTGtacctgcagatga |
| | | | acagcctgcgtgctgaggacactgccgtctattattgtGCAagaAATCTG |
| | | | gggCCTAGCttctatTTCgacTACtggggtcaaggaaccctggtcaccgt |
| | | | ctcctcgggtggtggtggatccggaggaggaggaAGTggaggtggaggtT |
| | | | CTgatatccagatgacccagtccccgagctccctgtccgcctctgtgggc |
| | | | gatagggtcaccatcacctgcAAAgccagtcaggatgtgTCCATTGGGgt |
| | | | agcctggtatcaacagaaaccaggaaaagctccgaaactactgatttact |
| | | | cggcatccTATCGCTACACGggagtcccttctcgcttctctggatccGGT |
| | | | tctgggacggatttcactctgaccatcagcagtctgcagccggaagactt |
| | | | cgcaacttattactgtcagcaaTACtatATCTATcctTACacgttcggac |
| | | | agggtaccaaggtggagatcaaa |
| Ab-HER- | 5137 | HC CDR1 | DTYIH |
| 69 | | (Kabat) | |
| | 5138 | HC CDR2 | RIYPTNGYTRYDPKFQD |
| | | (Kabat) | |
| | 5139 | HC CDR3 | WGGDGFYAMDY |
| | | (Kabat) | |
| | 5140 | HC CDR1 | GFNIKDT |
| | | (Chothia) | |
| | 5141 | HC CDR2 | YPTNGY |
| | | (Chothia) | |
| | 5142 | HC CDR3 | WGGDGFYAMDY |
| | | (Chothia) | |
| | 5130 | VH | QVQLQQSGPELVKPGASLKLSCTASGFNIKDTYIHWVKQRPEQGLEWIGR |
| | | | IYPTNGYTRYDPKFQDKATITADTSSNTAYLQVSRLTSEDTAVYYCSRWG |
| | | | GDGFYAMDYWGQGASVTVSS |
| | 5129 | DNA VH | CAGGTGCAGCTGCAGCAGAGCGGCCCCGAGCTGGTGAAGCCCGGCGCCAG |
| | | | CCTGAAGCTGAGCTGCACCGCCAGCGGCTTCAACATCAAGGACACCTACA |
| | | | TCCACTGGGTGAAGCAGAGACCCGAGCAGGGCCTGGAGTGGATCGGCAGA |
| | | | ATCTACCCCACCAACGGCTACACCAGATACGACCCCAAGTTCCAGGACAA |
| | | | GGCCACCATCACCGCCGACACCAGCAGCAACACCGCCTACCTGCAGGTGA |
| | | | GCAGACTGACCAGCGAGGACACCGCCGTGTACTACTGCAGCAGATGGGGC |
| | | | GGCGACGGCTTCTACGCCATGGACTACTGGGGCCAGGGCGCCAGCGTGAC |
| | | | CGTGAGCAGC |
| | 5132 | Heavy Chain | QVQLQQSGPELVKPGASLKLSCTASGFNIKDTYIHWVKQRPEQGLEWIGR |
| | | (hIgG1) | IYPTNGYTRYDPKFQDKATITADTSSNTAYLQVSRLTSEDTAVYYCSRWG |
| | | | GDGFYAMDYWGQGASVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 5131 | DNA Heavy | CAGGTGCAGCTGCAGCAGAGCGGCCCCGAGCTGGTGAAGCCCGGCGCCAG |
| | | Chain | CCTGAAGCTGAGCTGCACCGCCAGCGGCTTCAACATCAAGGACACCTACA |
| | | (hIgG1) | TCCACTGGGTGAAGCAGAGACCCGAGCAGGGCCTGGAGTGGATCGGCAGA |
| | | | ATCTACCCCACCAACGGCTACACCAGATACGACCCCAAGTTCCAGGACAA |
| | | | GGCCACCATCACCGCCGACACCAGCAGCAACACCGCCTACCTGCAGGTGA |
| | | | GCAGACTGACCAGCGAGGACACCGCCGTGTACTACTGCAGCAGATGGGGC |
| | | | GGCGACGGCTTCTACGCCATGGACTACTGGGGCCAGGGCGCCAGCGTGAC |
| | | | CGTGAGCAGCgcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaG |
| | | | agttgagcccaaatcttgtgacaaaactcacacatgcccaccgtgcccag |
| | | | cacctgaactcGtggggggaccgtcagtcttcctcCtccccccaaaaccc |
| | | | aaggacaccctcatgatctcccggacccctgaggtcacatgcgtggtggt |
| | | | ggacgtgagccacgaagaccctgaggtcaagttcaactggtacgtggacg |
| | | | gcgtggaggtgcataatgccaagacaaagccgcCggaggagcagtacaac |
| | | | agcacgCTccgtgtggtcagcgtcctcaccgtcctgcaccaggactggct |
| | | | gaatggcaaggagtacaagtgcaaggtctccaacaaagccccccagccc |
| | | | ccatcgagaaaaccatctccaaagccaaagggcagccccgagaaccacag |
| | | | gtgtacaccctgcccccatcccgggaTgagCtgaccaagaaccaggtcag |
| | | | cctgacctgcctggtcaaaggcttctatcccagcgacatcgccgtggagt |
| | | | gggagagcaatgggcagccggagaacaactacaagaccacgcctcTcgtg |
| | | | ctggactccgacggctccttcttcctctacagcaagctcaccgtggacaa |
| | | | gagcaggtggcagcaggggaacgtcttctcatgctccgtgatgcatgagg |
| | | | ctctgcacaaccactacacgcagaagagcctctccctgtctccgggtaaa |
| | 5143 | LC CDR1 | KASQDVNTAVA |
| | | (Kabat) | |
| | 5144 | LC CDR2 | SASFRYT |
| | | (Kabat) | |
| | 5145 | LC CDR3 | QQHYTTPPT |
| | | (Kabat) | |
| | 5146 | LC CDR1 | KASQDVNTAVA |
| | | (Chothia) | |
| | 5147 | LC CDR2 | SASFRYT |
| | | (Chothia) | |
| | 5148 | LC CDR3 | QQHYTTPPT |
| | | (Chothia) | |
| | 5134 | VL | DIVMTQSHKFMSTSVGDRVSITCKASQDVNTAVAWYQQKPGHSPKLLIYS |
| | | | ASFRYTGVPDRFTGSRSGTDFTFTISSVQAEDLAVYYCQQHYTTPPTFGG |
| | | | GTKVEIK |
| | 5133 | DNA VL | GACATCGTGATGACCCAGAGCCACAAGTTCATGAGCACCAGCGTGGGCGA |
| | | | CAGAGTGAGCATCACCTGCAAGGCCAGCCAGGACGTGAACACCGCCGTGG |
| | | | CCTGGTACCAGCAGAAGCCCGGCCACAGCCCCAAGCTGCTGATCTACAGC |
| | | | GCCAGCTTCAGATACACCGGCGTGCCGGACAGATTCACCGGCAGCAGAAG |
| | | | CGGCACCGACTTCACCTTCACCATCAGCAGCGTGCAGGCCGAGGACCTGG |
| | | | CCGTGTACTACTGCCAGCAGCACTACACCACCCCTCCTACCTTCGGCGGC |
| | | | GGCACCAAGGTGGAGATCAAG |
| | 5136 | Light Chain | DIVMTQSHKFMSTSVGDRVSITCKASQDVNTAVAWYQQKPGHSPKLLIYS |
| | | (hIgG1) | ASFRYTGVPDRFTGSRSGTDFTFTISSVQAEDLAVYYCQQHYTTPPTFGG |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5135 | DNA Light | GACATCGTGATGACCCAGAGCCACAAGTTCATGAGCACCAGCGTGGGCGA |
| | | Chain | CAGAGTGAGCATCACCTGCAAGGCCAGCCAGGACGTGAACACCGCCGTGG |
| | | (hIgG1) | CCTGGTACCAGCAGAAGCCCGGCCACAGCCCCAAGCTGCTGATCTACAGC |
| | | | GCCAGCTTCAGATACACCGGCGTGCCGGACAGATTCACCGGCAGCAGAAG |
| | | | CGGCACCGACTTCACCTTCACCATCAGCAGCGTGCAGGCCGAGGACCTGG |
| | | | CCGTGTACTACTGCCAGCAGCACTACACCACCCCTCCTACCTTCGGCGGC |
| | | | GGCACCAAGGTGGAGATCAAGAGAACCGTGGCCGCCCCTAGCGTGTTCAT |
| | | | CTTCCCGCCAAGCGACGAGCAGCTGAAGAGCGGCACCGCCAGCGTGGTGT |
| | | | GCCTGCTGAACAACTTCTACCCCAGAGAGGCCAAGGTGCAGTGGAAGGTG |
| | | | GACAACGCCCTGCAGAGCGGCAACAGCCAGGAGAGCGTGACCGAGCAGGA |
| | | | CAGCAAGGACAGCACCTACAGCCTGAGCAGCACCCTGACCCTGAGCAAGG |
| | | | CCGACTACGAGAAGCACAAGGTGTACGCCTGCGAGGTGACCCACCAGGGC |
| | | | CTGAGCAGCCCCGTGACCAAGAGCTTCAACAGAGGCGAGTGCt |
| AB-HER- | 5311 | HC CDR1 | GYFIN |
| 73 | | (Kabat) | |
| | 5312 | HC CDR2 | HISSSYATSTYNQKFQ |
| | | (Kabat) | |
| | 5313 | HC CDR3 | YCVRSGNY |
| | | (Kabat) | |
| | 5314 | HC CDR1 | GYSFTGY |
| | | (Chothia) | |
| | 5315 | HC CDR2 | SSSYA |
| | | (Chothia) | |
| | 5316 | HC CDR3 | YCVRSGNY |
| | | (Chothia) | |
| | 5358 | HC CDR1 | GYSFTGYFIN |
| | 5359 | HC CDR2 | HISSSYATSTYNQKFQG |
| | 5360 | HC CDR3 | SGNYEEYAMDY |
| | 5262 | VH | QVQLVQSGAEVKKPGASVKVSCKASGYSFTGYFINWVREAPGQGLEWMGH |
| | | | ISSSYATSTYNQKFQGRVTFTVDTSSSTAYMELSSLRSEDTAVYYCVRSG |
| | | | NYEEYAMDYWGQGTLVTVSS |
| | 5261 | DNA VH | CAAgttcagctggtgCAGtctggTGCAGAAGTTAAGAAGccagggGCAtc |
| | | | aGTAAAGGTGTCCtgCAAGgcttctggcTACTCTTTCACTGGGTATTTCa |
| | | | taAATtgggtgAGAGAGgccccCGGCCAGggcctggaatggATGGGCCAT |
| | | | attTCCTCCTCTTATGCAACTTCTACAtatAACCAGAAGTTTCAGggcAG |
| | | | AGTCactTTCACTGTGgacacatccAGCTCTacagcctacATGGAGCTGT |
| | | | CAagcctgAGATCTgaggacactgcagtctattattgtGTGagaAGTgga |
| | | | AACTAtGAAGAGTATgctatggacTACtggggtcaaggaaccctggtcac |
| | | | AgtctcctcA |
| | 5264 | Heavy Chain | QVQLVQSGAEVKKPGASVKVSCKASGYSFTGYFINWVREAPGQGLEWMGH |
| | | (hIgG1) | ISSSYATSTYNQKFQGRVTFTVDTSSSTAYMELSSLRSEDTAVYYCVRSG |
| | | | NYEEYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPPKSCDKTHTCPPCPAPELLGGPSVFLFPPK |
| | | | PKDTLMISRTPEVTCVVVDVSHEDPEVKFNWYVDGVEVHNAKTKPREEQY |
| | | | NSTYRVVSVLTVLHQDWLNGKEYKCKVSNKALPAPIEKTISKAKGQPREP |
| | | | QVYTLPPSRDELTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPG |
| | | | KRKRREGRGSLLTCGDVEENPGPMDMRVPAQLLGLLLLWLSGARCEVQLV |
| | | | ESGGGLVQPGGSLRLSCAASGFNIKDTYIHWVRQAPGKGLEWVARIYPTN |
| | | | GYTRYADSVKGRFTISADTSKNTAYLQMNSLRAEDTAVYYCSRWGGDGFY |
| | | | AMDYWGQGTLVTVSS |
| | 5263 | DNA Heavy | CAAgttcagctggtgCAGtctggTGCAGAAGTTAAGAAGccagggGCAtc |
| | | Chain | aGTAAAGGTGTCCtgCAAGgcttctggcTACTCTTTCACTGGGTATTTCa |
| | | (hIgG1) | taAATtgggtgAGAGAGgccccCGGCCAGggcctggaatggATGGGCCAT |
| | | | attTCCTCCTCTTATGCAACTTCTACAtatAACCAGAAGTTTCAGggcAG |
| | | | AGTCactTTCACTGTGgacacatccAGCTCTacagcctacATGGAGCTGT |
| | | | CAagcctgAGATCTgaggacactgcagtctattattgtGTGagaAGTgga |
| | | | AACTAtGAAGAGTATgctatggacTACtggggtcaaggaaccctggtcac |
| | | | AgtctcctcAgcCtcTaccaagggcccatcTgtcttccccctggcaccct |
| | | | cctccaagagcacctctgggggcacagcAgccctgggctgcctggtcaag |
| | | | gactacttcccTgaacctgtgacAgtctcTtggaactcaggGgccctgac |
| | | | cTCTggGgtgcacaccttcccAgctgtcctacagtcctcaggactctact |
| | | | ccctcTCTTCTgtggtgacAgtgccctccagcagcttgggcacccagacc |
| | | | tacatctgcaaTgtgaatcacaagcccagcaacaccaaggtggacaagaa |
| | | | agttgagcccCCCaaatcttgtgacaaaactcacacatgcccaccCtgcc |
| | | | cagcacctgaactcctggggggaccCtcagtcttcctcttccccccaaaa |
| | | | cccaaggacaccctcatgatctccAGAacccctgaggtcacatgTgtggt |
| | | | ggtggaTgtgagccaTgaagaccctgaggtcaagttcaactggtaTgtgg |
| | | | aTggGgtggaggtgcataatgccaagacaaagccCAGAgaggagcagtac |
| | | | aacagcacCtacAGAgtggtcTCAgtcctcacTgtcctgcaccaggactg |
| | | | gctgaatggcaaggagtacaagtgcaaggtctccaacaaagccctcccag |
| | | | cccccatAgagaaaaccatctccaaagccaaagggcagcccAGAgaacca |
| | | | caggtgtacaccctgcccccatccAGAgatgagctgaccaagaaccaggt |
| | | | cagcctgacctgcctggtcaaaggcttctatcccTCTgacatTgcTgtgg |
| | | | agtgggagagcaatgggcagccAgagaacaactacaagaccacCcctccA |
| | | | gtgctggactcTgatggctccttcttcctctacagcaagctcacAgtgga |
| | | | caagagcaggtggcagcaggggaaTgtcttctcatgctcIgtgatgcatg |
| | | | aggctctgcacaaccactacacTcagaagagcctctccctgtctccTggt |
| | | | aaa |
| | 5355 | LC CDR1 | RSSQTLLYSNNQKNYLA |
| | 5356 | LC CDR2 | WAFTRKS |
| | 5357 | LC CDR3 | QQYSNYPWT |
| | 5354 | VL | DIVLTQSPDSLAVSLGERATINCRSSQTLLYSNNQKNYLAWYQKKPGQPP |
| | | | KLLISWAFTRKSGVPDRFSGSGSGTDFTLTISSLQAEDVAVYYCQQYSNY |
| | | | PWTFGQGTKVEIK |
| | 5353 | DNA VL | gatatTGTACTGacccagtccccTGACtccctgGCTGTGtctCTGggGGA |
| | | | AaggGCAaccatcAACtgcAGGAGTagtcagACCCTTCTGTACTCTAATA |
| | | | ACCAGAAAAACTACCTGgcctggtatcaaAAAaaaccaggaCAGCCTccT |
| | | | aaactactgattAGTTGGgcaTTCACTAGGAAGtctggagtccctGACAG |
| | | | GttctctggatcTGGGtcTggAacAgatttcacActgaccatcagcagtc |
| | | | tgcagGCTgaagaTGTTgcaGTCtaCtactgtcagcaaTACTCAAACTAC |
| | | | cctTGGacTttTggacagggGaccaaAgtggagatcaaa |
| | 5268 | Light Chain | DIVLTQSPDSLAVSLGERATINCRSSQTLLYSNNQKNYLAWYQKKPGQPP |
| | | (hIgG1) | KLLISWAFTRKSGVPDRFSGSGSGTDFTLTISSLQAEDVAVYYCQQYSNY |
| | | | PWTFGQGTKVEIKRTVAAPSVFIFPPSDEQLKSGTASVKCLLNNFYPREA |
| | | | KVQWKVDNALQSGNSQESVTEQDSKDSTYSLSSTLTLSKADYEKHKVYAC |
| | | | EVTHQGLSSPVTKSENRGEC |
| | 5267 | DNA Light | gatatTGTACTGacccagtccccTGACtccctgGCTGTGtctCTGggGGA |
| | | Chain | AaggGCAaccatcAACtgcAGGAGTagtcagACCCTTCTGTACTCTAATA |
| | | (hIgG1) | ACCAGAAAAACTACCTGgcctggtatcaaAAAaaaccaggaCAGCCTccT |
| | | | aaactactgattAGTTGGgcaTTCACTAGGAAGtctggagtccctGACAG |
| | | | GttctctggatcTGGGtcTggAacAgatttcacActgaccatcagcagtc |
| | | | tgcagGCTgaagaTGTTgcaGTCtaCtactgtcagcaaTACTCAAACTAC |
| | | | cctTGGacTttIggacagggGaccaaAgtggagatcaaaAGGactgtCgc |
| | | | tgcaccatctgtcttcatcttcccAccatctgatgagcagttgaaatctg |
| | | | gaactgcctctgttAAGtgcctgctgaataacttctatcccagagaggcc |
| | | | aaagtacagtggaaggtggataaTgccctccaaAGTggtaactcccagga |
| | | | gagtgtcacagagcaggacagcaaggacagcacctacagcctcagcagca |
| | | | ccctgacCctgagcaaagcagactaTgagaaacacaaagtctaTgcctgT |
| | | | gaagtcacccatcagggcctgagcAGCccAgtcacaaagagcttcaacag |
| | | | gggagagtgt |
| | 5037 | HC CDR1 | DTYIH |
| | | (Kabat) | |
| | 5038 | HC CDR2 | RIYPTNGYTRYADSVKG |
| | | (Kabat) | |
| | 5039 | HC CDR3 | WGGDGFYAMDY |
| | | (Kabat) | |
| | 5040 | HC CDR1 | GFNIKDT |
| | | (Chothia) | |
| | 5041 | HC CDR2 | YPTNGY |
| | | (Chothia) | |
| | 5042 | HC CDR3 | WGGDGFYAMDY |
| | | (Chothia) | |
| | 5361 | HC CDR1 | GFNIKDTYIH |
| | 5362 | HC CDR2 | RIYPTNGYTRYADSVKG |
| | 5363 | HC CDR3 | WGGDGFYAMDY |
| | 5290 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 6552 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | ggggaTggcttctatgctatggacTACtggggtcaaggaaccctggtcac |
| | | | AgtctcctcT |
| | 5317 | LC CDR1 | RASQDVNTAVA |
| | | (Kabat) | |
| | 5318 | LC CDR2 | SASFLYS |
| | | (Kabat) | |
| | 5319 | LC CDR3 | QQHYTTPPT |
| | | Kabat) | |
| | 5320 | LC CDR1 | RASQDVNTAVA |
| | | (Chothia) | |
| | 5321 | LC CDR2 | SASFLYS |
| | | (Chothia) | |
| | 5322 | LC CDR3 | QQHYTTPPT |
| | | (Chothia) | |
| | 5266 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5265 | DNA VL | gatatccagatgacccagtccccAagctccctgtcTgcctctgtgggGga |
| | | | tagggtcaccatcacctgcAGAgccagtcaggatgtgaatactgctgtag |
| | | | cctggtatcaacagaaaccaggaaaagctccCaaactactgatttactcT |
| | | | gcatccttcctcTACtctggagtcccttctAGAttctctggatccagatc |
| | | | tggCacTgaCttcactctgaccatcagTagtctgcagccTgaagacttTg |
| | | | caacttattactgtcaAcaacattatactactcctcccacCttTggacag |
| | | | ggtaccaaggtggagatcaaa |
| | 5351 | scFv | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSSGGGGSGGGGSGGGGSDIQMTQSPSSLSASV |
| | | | GDRVTITCRASQDVNTAVAWYQQKPGKAPKLLIYSASFLYSGVPSRFSGS |
| | | | RSGTDFTLTISSLQPEDFATYYCQQHYTTPPTFGQGTKVEIK |
| | 5364 | scFv DNA | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | ggggaTggcttctatgctatggacTACtggggtcaaggaaccctggtcac |
| | | | AgtctcctcTggtggtggtggatcTggaggaggaggaAGTggaggtggag |
| | | | gtTCTgatatccagatgacccagtccccAagctccctgtcTgcctctgtg |
| | | | ggGgatagggtcaccatcacctgcAGAgccagtcaggatgtgaatactgc |
| | | | tgtagcctggtatcaacagaaaccaggaaaagctccCaaactactgattt |
| | | | actcTgcatccttcctcTACtctggagtcccttctAGAttctctggatcc |
| | | | agatctggCacTgaCttcactctgaccatcagTagtctgcagccTgaaga |
| | | | cttTgcaacttattactgtcaAcaacattatactactcctcccacCttTg |
| | | | gacagggtaccaaggtggagatcaaa |
| | 5365 | Light Chain | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSSGGGGSGGGGSGGGGSDIQMTQSPSSLSASV |
| | | | GDRVTITCRASQDVNTAVAWYQQKPGKAPKLLIYSASFLYSGVPSRFSGS |
| | | | RSGTDFTLTISSLQPEDFATYYCQQHYTTPPTFGQGTKVEIKGSDIVLTQ |
| | | | SPDSLAVSLGERATINCRSSQTLLYSNNQKNYLAWYQKKPGQPPKLLISW |
| | | | AFTRKSGVPDRFSGSGSGTDFTLTISSLQAEDVAVYYCQQYSNYPWTFGQ |
| | | | GTKVEIKRTVAAPSVFIFPPSDEQLKSGTASVKCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5366 | DNA Light | gaggttcagctggtggagtctggTggtggcctGgtgcagccagggggctc |
| | | Chain | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | ggggaTggcttctatgctatggacTACtggggtcaaggaaccctggtcac |
| | | | AgtctcctcTggtggtggtggatcTggaggaggaggaAGTggaggtggag |
| | | | gtTCTgatatccagatgacccagtccccAagctccctgtcTgcctctgtg |
| | | | ggGgatagggtcaccatcacctgcAGAgccagtcaggatgtgaatactgc |
| | | | tgtagcctggtatcaacagaaaccaggaaaagctccCaaactactgattt |
| | | | actcTgcatccttcctcTACtctggagtcccttctAGAttctctggatcc |
| | | | agatctggCacTgaCttcactctgaccatcagTagtctgcagccTgaaga |
| | | | cttTgcaacttattactgtcaAcaacattatactactcctcccacCttTg |
| | | | gacagggtaccaaggtggagatcaaaGGCTCTgatatTGTACTGacccag |
| | | | tccccTGACtccctgGCTGTGtctCTGggGGAAaggGCAaccatcAACtg |
| | | | CAGGAGTagtcagACCCTTCTGTACTCTAATAACCAGAAAAACTACCTGg |
| | | | cctggtatcaaAAAaaaccaggaCAGCCTccTaaactactgattAGTTGG |
| | | | gcaTTCACTAGGAAGtctggagtccctGACAGGttctctggatcTGGGtc |
| | | | TggAacAgatttcacActgaccatcagcagtctgcagGCTgaagaTGTTg |
| | | | caGTCtaCtactgtcagcaaTACTCAAACTACcctTGGacTttTggacag |
| | | | ggGaccaaAgtggagatcaaaAGGactgtCgctgcaccatctgtcttcat |
| | | | cttcccAccatctgatgagcagttgaaatctggaactgcctctgttAAGt |
| | | | gcctgctgaataacttctatcccagagaggccaaagtacagtggaaggtg |
| | | | gataaTgccctccaaAGTggtaactcccaggagagtgtcacagagcagga |
| | | | cagcaaggacagcacctacagcctcagcagcaccctgacCctgagcaaag |
| | | | cagactaTgagaaacacaaagtctaTgcctgTgaagtcacccatcagggc |
| | | | ctgagcAGCccAgtcacaaagagcttcaacaggggagagtgt |
| Ab-HER- | 5281 | HC CDR1 | DTYIH |
| 75 | | (Kabat) | |
| | 5282 | HC CDR2 | RIYPTNGYTRYADSVKG |
| | | (Kabat) | |
| | 5283 | HC CDR3 | WGGDGFYAMDY |
| | | (Kabat) | |
| | 5284 | HC CDR1 | GFNIKDT |
| | | (Chothia) | |
| | 5285 | HC CDR2 | YPTNGY |
| | | (Chothia) | |
| | 5286 | HC CDR3 | WGGDGFYAMDY |
| | | (Chothia) | |
| | 5270 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5269 | DNA VH | gaggttcagctggtggagtctggAggtggcctggtgcagccagggggctc |
| | | | actcAGAttgtcctgtgcagcttctggcttcaacattaaagacacctata |
| | | | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | ggggaTggcttctatgctatggacTACtggggtcaaggaaccctggtcac |
| | | | AgtctcctcT |
| | 5272 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 7589 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMASRTPEVTCVVVDVSHEDPEVKFNWYVDGVEVHNAKTKPPEEQYN |
| | | | STLRVVSVLTVLAQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNAYTQKSLSLSPGK |
| | 5271 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | ggggaTggcttctatgctatggacTACtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccIgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaIgtgaatcacaagcccagcaacaccaaggggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatIgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |
| | 5244 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | ggggaTggcttctatgctatggacTACtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccIgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatTgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcagAtggcagcaggggaaTgtcttctcatgcTCTgtgatgcatgagg |
| | | | ctctgcacaaccactacacCcagaagagcctctccctgtctccAggtaaa |
| | 7590 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | ggggaTggcttctatgctatggacTACtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccTgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgGCAtccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgGCCcaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatTgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcagAtggcagcaggggaaTgtcttctcatgcTCTgtgatgcatgagg |
| | | | ctctgcacaacGCTtacacCcagaagagcctctccctgtctccAggtaaa |
| | 5287 | LC CDR1 | RASQDVNTAVA |
| | | (Kabat) | |
| | 5288 | LC CDR2 | SASFLYS |
| | | (Kabat) | |
| | 5289 | LC CDR3 | QQHYTTPPT |
| | | (Kabat) | |
| | 5287 | LC CDR1 | RASQDVNTAVA |
| | | (Chothia) | |
| | 5291 | LC CDR2 | SASFLYS |
| | | (Chothia) | |
| | 5292 | LC CDR3 | QQHYTTPPT |
| | | (Chothia) | |
| | 5274 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5273 | DNA VL | gatatccagatgacccagtccccTagctccctgtcTgcctctgtgggAga |
| | | | tagggtcaccatcacctgcAGAgccagtcaggatgtgaatactgctgtag |
| | | | cctggtatcaacagaaaccaggaaaagctccTaaactactgatttactcT |
| | | | gcatccttcctcTACtctggagtcccttctAGGttctctggatccagatc |
| | | | tgggacTgatttcactctgaccatcagcagtctgcagccAgaagacttTg |
| | | | caacttattactgtcagcaacattatactactcctcccacIttIggacag |
| | | | ggtaccaaggtggagatcaaa |
| | 5245 | DNA VL | gatatccagatgacccagtccccTagctccctgtcTgcctctgtgggAga |
| | | | tagggtcaccatcacctgTAGAgccagtcaggatgtgaatactgctgtag |
| | | | cctggtatcaacagaaaccaggaaaagctccTaaactactgatttactcT |
| | | | gcatccttcctcTACtctggagtcccttctAGGttctctggatccagatc |
| | | | tgggacTgatttcactctgaccatcagcagtctgcagccAgaagacttTg |
| | | | caacttattactgtcagcaacattatactactcctcccacTttTggacag |
| | | | ggtaccaaggtggagatcaaa |
| | 5276 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5246 | DNA Light | gatatccagatgacccagtccccTagctccctgtcTgcctctgtgggAga |
| | | Chain | tagggtcaccatcacctgTAGAgccagtcaggatgtgaatactgctgtag |
| | | (hIgG1) | cctggtatcaacagaaaccaggaaaagctccTaaactactgatttactcT |
| | | | gcatccttcctcTACtctggagtcccttctAGGttctctggatccagatc |
| | | | tgggacTgatttcactctgaccatcagcagtctgcagccAgaagacttTg |
| | | | caacttattactgtcagcaacattatactactcctcccacTttTggacag |
| | | | ggtaccaaggtggagatcaaaAGAactgtggctgcaccatctgtcttcat |
| | | | cttcccAccatctgatgagcagttgaaatctggaactgcctctgttgtgt |
| | | | gcctgctgaataacttctatcccagagaggccaaagtacagtggaaggtg |
| | | | gataaTgccctccaatcAggtaactcccaggagagtgtcacagagcagga |
| | | | cagcaaggacagcacctacagcctcagcagcaccctgacTctgagcaaag |
| | | | cagactaTgagaaacacaaagtctaIgcctgTgaagtcacccatcagggc |
| | | | ctgagctcTccTgtcacaaagagcttcaacaggggagagtgt |
| | 5275 | DNA Light | gatatccagatgacccagtccccTagctccctgtcTgcctctgtgggAga |
| | | Chain | tagggtcaccatcacctgcAGAgccagtcaggatgtgaatactgctgtag |
| | | (hIgG1) | cctggtatcaacagaaaccaggaaaagctccTaaactactgatttactcT |
| | | | gcatccttcctcTACtctggagtcccttctAGGttctctggatccagatc |
| | | | tgggacTgatttcactctgaccatcagcagtctgcagccAgaagacttTg |
| | | | caacttattactgtcagcaacattatactactcctcccacTttIggacag |
| | | | ggtaccaaggtggagatcaaaAGAactgtggctgcaccatctgtcttcat |
| | | | cttcccAccatctgatgagcagttgaaatctggaactgcctctgttgtgt |
| | | | gcctgctgaataacttctatcccagagaggccaaagtacagtggaaggtg |
| | | | gataaTgccctccaatcAggtaactcccaggagagtgtcacagagcagga |
| | | | cagcaaggacagcacctacagcctcagcagcaccctgacTctgagcaaag |
| | | | cagactaTgagaaacacaaagtctaTgcctgTgaagtcacccatcagggc |
| | | | ctgagctcTccTgtcacaaagagcttcaacaggggagagtgt |
| Ab-HER- | 5323 | HC CDR1 | DTYIH |
| 77 | | (Kabat) | |
| | 5324 | HC CDR2 | RIYPINGYTRYADSVKG |
| | | (Kabat) | |
| | 5325 | HC CDR3 | WGGDGFYAMDY |
| | | (Kabat) | |
| | 5326 | HC CDR1 | GFNIKDT |
| | | (Chothia) | |
| | 5327 | HC CDR2 | YPTNGY |
| | | (Chothia) | |
| | 5325 | HC CDR3 | WGGDGFYAMDY |
| | | (Chothia) | |
| | 5329 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5330 | DNA VH | GAAGTGCAGCTGGTGGAAAGCGGCGGAGGACTGGTGCAGCCTGGCGGATC |
| | | | TCTGAGACTGAGCTGTGCCGCCAGCGGCTTCAACATCAAGGACACCTACA |
| | | | TCCACTGGGTGCGCCAGGCCCCTGGCAAGGGACTGGAATGGGTGGCCAGA |
| | | | ATCTACCCCACCAACGGCTACACCAGATACGCCGACAGCGTGAAGGGCCG |
| | | | GTTCACCATCAGCGCCGACACCAGCAAGAACACCGCCTACCTGCAGATGA |
| | | | ACAGCCTGCGGGCCGAGGACACCGCCGTGTACTACTGTAGTAGATGGGGA |
| | | | GGCGACGGCTTCTACGCCATGGACTATTGGGGCCAGGGCACCCTCGTGAC |
| | | | AGTGTCTAGT |
| | 5331 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKKVEPKSCDKTHTCPPCPAPELLGGPSVFLFPPKP |
| | | | KDTLMISRTPEVTCVVVDVSHEDPEVKFNWYVDGVEVHNAKTKPREEQYN |
| | | | STYRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| | 5332 | DNA Heavy | GAAGTGCAGCTGGTGGAAAGCGGCGGAGGACTGGTGCAGCCTGGCGGATC |
| | | Chain | TCTGAGACTGAGCTGTGCCGCCAGCGGCTTCAACATCAAGGACACCIACA |
| | | (hIgG1) | TCCACTGGGTGCGCCAGGCCCCTGGCAAGGGACTGGAATGGGTGGCCAGA |
| | | | ATCTACCCCACCAACGGCTACACCAGATACGCCGACAGCGTGAAGGGCCG |
| | | | GTTCACCATCAGCGCCGACACCAGCAAGAACACCGCCTACCTGCAGATGA |
| | | | ACAGCCTGCGGGCCGAGGACACCGCCGTGTACTACTGTAGTAGATGGGGA |
| | | | GGCGACGGCTTCTACGCCATGGACTATTGGGGCCAGGGCACCCTCGTGAC |
| | | | AGTGTCTAGTGCGTCGACCAAGGGGCCCTCGGTCTTCCCCCTGGCACCCT |
| | | | CCTCCAAGAGCACCTCTGGGGGCACAGCGGCCCTGGGCTGCCTGGTCAAG |
| | | | GACTACTTCCCCGAACCGGTGACGGTGTCGTGGAACTCAGGCGCCCTGAC |
| | | | CAGCGGCGTGCACACCTTCCCGGCTGTCCTACAGTCCTCAGGACTCTACT |
| | | | CCCTCAGCAGCGTGGTGACCGTGCCCTCCAGCAGCTTGGGCACCCAGACC |
| | | | TACATCTGCAACGTGAATCACAAGCCCAGCAACACCAAGGTGGACAAGAA |
| | | | AGTTGAGCCCAAATCTTGTGACAAAACTCACACATGCCCACCGTGCCCAG |
| | | | CACCTGAACTCCTGGGGGGACCGTCAGTCTTCCTCTTCCCCCCAAAACCC |
| | | | AAGGACACCCTCATGATCTCCCGGACCCCTGAGGTCACATGCGTGGTGGT |
| | | | GGACGTGAGCCACGAAGACCCTGAGGTCAAGTTCAACTGGTACGTGGACG |
| | | | GCGTGGAGGTGCATAATGCCAAGACAAAGCCGCGGGAGGAGCAGTACAAC |
| | | | AGCACGTACCGTGTGGTCAGCGTCCTCACCGTCCTGCACCAGGACTGGCT |
| | | | GAATGGCAAGGAGTACAAGTGCAAGGTCTCCAACAAAGCCCTCCCAGCCC |
| | | | CCATCGAGAAAACCATCTCCAAAGCCAAAGGGCAGCCCCGAGAACCACAG |
| | | | GTGTACACCCTGCCCCCATCCCGGGATGAGCTGACCAAGAACCAGGTCAG |
| | | | CCTGACCTGCCTGGTCAAAGGCTTCTATCCCAGCGACATCGCCGTGGAGT |
| | | | GGGAGAGCAATGGGCAGCCGGAGAACAACTACAAGACCACGCCTCCCGTG |
| | | | CTGGACTCCGACGGCTCCTTCTTCCTCTACAGCAAGCTCACCGTGGACAA |
| | | | GAGCAGGTGGCAGCAGGGGAACGTCTTCTCATGCTCCGTGATGCATGAGG |
| | | | CTCTGCACAACCACTACACGCAGAAGAGCCTCTCCCTGTCTCCCGGCAAG |
| | 5333 | LC CDR1 | RASQDVNTAVA |
| | | (Kabat) | |
| | 5334 | LC CDR2 | SASFLYS |
| | | (Kabat) | |
| | 5335 | LC CDR3 | QQHYTTPPT |
| | | (Kabat) | |
| | 5336 | LC CDR1 | RASQDVNTAVA |
| | | (Chothia) | |
| | 5337 | LC CDR2 | SASFLYS |
| | | (Chothia) | |
| | 5338 | LC CDR3 | QQHYTTPPT |
| | | (Chothia) | |
| | 5339 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5340 | DNA VL | GATATCCAGATGACCCAGAGCCCCAGCAGCCTGTCTGCCAGCGTGGGCGA |
| | | | CAGAGTGACCATCACCTGTAGAGCCAGCCAGGACGTGAACACCGCCGTGG |
| | | | CCTGGTATCAGCAGAAGCCTGGCAAGGCCCCCAAGCTGCTGATCTACAGC |
| | | | GCCAGCTTCCTGTACAGCGGCGTGCCCAGCAGATTCAGCGGCAGCAGATC |
| | | | CGGCACCGACTTCACCCTGACCATCAGCTCCCTGCAGCCCGAGGACTTCG |
| | | | CCACCTACTACTGCCAGCAGCACTACACCACCCCCCCCACATTTGGCCAG |
| | | | GGCACCAAGGTGGAAATCAAG |
| | 5341 | Light Chain | DIQMTQSPSSLSASVGDRVTITCRASQDVNTAVAWYQQKPGKAPKLLIYS |
| | | (hIgG1) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSFNRGEC |
| | 5342 | DNA Light | GATATCCAGATGACCCAGAGCCCCAGCAGCCTGTCTGCCAGCGTGGGCGA |
| | | Chain | CAGAGTGACCATCACCTGTAGAGCCAGCCAGGACGTGAACACCGCCGTGG |
| | | (hIgG1) | CCTGGTATCAGCAGAAGCCTGGCAAGGCCCCCAAGCTGCTGATCTACAGC |
| | | | GCCAGCTTCCTGTACAGCGGCGTGCCCAGCAGATTCAGCGGCAGCAGATC |
| | | | CGGCACCGACTTCACCCTGACCATCAGCTCCCTGCAGCCCGAGGACTTCG |
| | | | CCACCTACTACTGCCAGCAGCACTACACCACCCCCCCCACATTTGGCCAG |
| | | | GGCACCAAGGTGGAAATCAAGCGTACGGTGGCTGCACCATCTGTCTTCAT |
| | | | CTTCCCGCCATCTGATGAGCAGTTGAAATCTGGAACTGCCTCTGTTGTGT |
| | | | GCCTGCTGAATAACTTCTACCCCAGAGAAGCCAAAGTGCAGTGGAAGGTG |
| | | | GACAACGCCCTGCAGAGCGGAAACAGCCAGGAAAGCGTGACAGAGCAGGA |
| | | | TTCCAAGGATTCCACATACAGCCTGAGCAGCACACTGACACTGTCCAAGG |
| | | | CCGACTACGAGAAGCACAAGGTGTACGCCTGCGAAGTGACACACCAGGGA |
| | | | CTGTCCTCCCCTGTGACAAAGAGCTTCAACAGAGGAGAATGC |
| Ab-HER- | 5311 | HC CDR1 | GYFIN |
| 78 | | (Kabat) | |
| | 5312 | HC CDR2 | HISSSYATSTYNQKFQ |
| | | (Kabat) | |
| | 5313 | HC CDR3 | YCVRSGNY |
| | | (Kabat) | |
| | 5314 | HC CDR1 | GYSFTGY |
| | | (Chothia) | |
| | 5315 | HC CDR2 | SSSYA |
| | | (Chothia | |
| | 5316 | HC CDR3 | YCVRSGNY |
| | | (Chothia) | |
| | 5358 | HC CDR1 | GYSFTGYFIN |
| | 5359 | HC CDR2 | HISSSYATSTYNQKFQG |
| | 5360 | HC CDR3 | SGNYEEYAMDY |
| | 5262 | VH | QVQLVQSGAEVKKPGASVKVSCKASGYSFTGYFINWVREAPGQGLEWMGH |
| | | | ISSSYATSTYNQKFQGRVTFTVDTSSSTAYMELSSLRSEDTAVYYCVRSG |
| | | | NYEEYAMDYWGQGTLVTVSS |
| | 5261 | DNA VH | CAAgttcagctggtgCAGtctggTGCAGAAGTTAAGAAGccagggGCAtc |
| | | | aGTAAAGGTGTCCtgCAAGgcttctggcTACTCTTTCACTGGGTATTTCa |
| | | | taAATtgggtgAGAGAGgccccCGGCCAGggcctggaatggATGGGCCAT |
| | | | attTCCTCCTCTTATGCAACTTCTACAtatAACCAGAAGTTTCAGggcAG |
| | | | AGTCactTTCACTGTGgacacatccAGCTCTacagcctacATGGAGCTGT |
| | | | CAagcctgAGATCTgaggacactgcagtctattattgtGTGagaAGTgga |
| | | | AACTAtGAAGAGTATgctatggacTACtggggtcaaggaaccctggtcac |
| | | | AgtctcctcA |
| | 5376 | Heavy Chain | QVQLVQSGAEVKKPGASVKVSCKASGYSFTGYFINWVREAPGQGLEWMGH |
| | | (hIgG1) | ISSSYATSTYNQKFQGRVTFTVDTSSSTAYMELSSLRSEDTAVYYCVRSG |
| | | | NYEEYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 5377 | DNA Heavy | CAAgttcagctggtgCAGtctggTGCAGAAGTTAAGAAGccagggGCAtc |
| | | Chain | aGTAAAGGTGTCCtgCAAGgcttctggcTACTCTTTCACTGGGTATTTCa |
| | | (hIgG1) | taAATtgggtgAGAGAGgccccCGGCCAGggcctggaatggATGGGCCAT |
| | | | attTCCTCCTCTTATGCAACTTCTACAtatAACCAGAAGTTTCAGggcAG |
| | | | AGTCactTTCACTGTGgacacatccAGCTCTacagcctacATGGAGCTGT |
| | | | CAagcctgAGATCTgaggacactgcagtctattattgtGTGagaAGTgga |
| | | | AACTAtGAAGAGTATgctatggacTACtggggtcaaggaaccctggtcac |
| | | | AgtctcctcAgcCtcTaccaagggcccatcTgtcttccccctggcaccct |
| | | | cctccaagagcacctctgggggcacagcTgccctgggctgcctggtcaag |
| | | | gactacttcccIgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatTgcTgtggagt |
| | | | gggagagcaatgggcagccTgagaacaactacaagaccacAcctcTTgtg |
| | | | ctggactcTgaIggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |
| | 5355 | LC CDR1 | RSSQTLLYSNNQKNYLA |
| | 5356 | LC CDR2 | WAFTRKS |
| | 5357 | LC CDR3 | QQYSNYPWT |
| | 5354 | VL | DIVLTQSPDSLAVSLGERATINCRSSQTLLYSNNQKNYLAWYQKKPGQPP |
| | | | KLLISWAFTRKSGVPDRFSGSGSGTDFTLTISSLQAEDVAVYYCQQYSNY |
| | | | PWTFGQGTKVEIK |
| | 5353 | DNA VL | gatatTGTACTGacccagtccccTGACtccctgGCTGTGtctCTGggGGA |
| | | | AaggGCAaccatcAACtgcAGGAGTagtcagACCCTTCTGTACTCTAATA |
| | | | ACCAGAAAAACTACCTGgcctggtatcaaAAAaaaccaggaCAGCCTccT |
| | | | aaactactgattAGTTGGgcaTTCACTAGGAAGtctggagtccctGACAG |
| | | | GttctctggatcTGGGtcTggAacAgatttcacActgaccatcagcagtc |
| | | | tgcagGCTgaagaTGTTgcaGTCtaCtactgtcagcaaTACTCAAACTAC |
| | | | cctTGGacTttTggacagggGaccaaAgtggagatcaaa |
| | 5268 | Light Chain | DIVLTQSPDSLAVSLGERATINCRSSQTLLYSNNQKNYLAWYQKKPGQPP |
| | | (hIgG1) | KLLISWAFTRKSGVPDRFSGSGSGTDFTLTISSLQAEDVAVYYCQQYSNY |
| | | | PWTFGQGTKVEIKRTVAAPSVFIFPPSDEQLKSGTASVKCLLNNFYPREA |
| | | | KVQWKVDNALQSGNSQESVTEQDSKDSTYSLSSTLTLSKADYEKHKVYAC |
| | | | EVTHQGLSSPVTKSENRGEC |
| | 5267 | DNA Light | gatatTGTACTGacccagtccccTGACtccctgGCTGTGtctCTGggGGA |
| | | Chain | AaggGCAaccatcAACtgcAGGAGTagtcagACCCTTCTGTACTCTAATA |
| | | (hIgG1) | ACCAGAAAAACTACCTGgcctggtatcaaAAAaaaccaggaCAGCCTccT |
| | | | aaactactgattAGTTGGgcaTTCACTAGGAAGtctggagtccctGACAG |
| | | | GttctctggatcTGGGtcTggAacAgatttcacActgaccatcagcagtc |
| | | | tgcagGCTgaagaTGTTgcaGTCtaCtactgtcagcaaTACTCAAACTAC |
| | | | cctTGGacTttIggacagggGaccaaAgtggagatcaaaAGGactgtCgc |
| | | | tgcaccatctgtcttcatcttcccAccatctgatgagcagttgaaatctg |
| | | | gaactgcctctgttAAGtgcctgctgaataacttctatcccagagaggcc |
| | | | aaagtacagtggaaggtggataaTgccctccaaAGTggtaactcccagga |
| | | | gagtgtcacagagcaggacagcaaggacagcacctacagcctcagcagca |
| | | | ccctgacCctgagcaaagcagactaTgagaaacacaaagtctaTgcctgT |
| | | | gaagtcacccatcagggcctgagcAGCccAgtcacaaagagcttcaacag |
| | | | gggagagtgt |
| | 5037 | HC CDR1 | DTYIH |
| | | (Kabat) | |
| | 5038 | HC CDR2 | RIYPTNGYTRYADSVKG |
| | | (Kabat) | |
| | 5039 | HC CDR3 | WGGDGFYAMDY |
| | | (Kabat) | |
| | 5040 | HC CDR1 | GFNIKDT |
| | | (Chothia) | |
| | 5041 | HC CDR2 | YPTNGY |
| | | (Chothia) | |
| | 5042 | HC CDR3 | WGGDGFYAMDY |
| | | (Chothia) | |
| | 5361 | HC CDR1 | GFNIKDTYIH |
| | 5362 | HC CDR2 | RIYPTNGYTRYADSVKG |
| | 5363 | HC CDR3 | WGGDGFYAMDY |
| | 5290 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 6552 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | ggggaTggcttctatgctatggacTACtggggtcaaggaaccctggtcac |
| | | | AgtctcctcT |
| | 5317 | LC CDR1 | RASQDVNTAVA |
| | | (Kabat) | |
| | 5318 | LC CDR2 | SASFLYS |
| | | (Kabat) | |
| | 5319 | LC CDR3 | QQHYTTPPT |
| | | (Kabat) | |
| | 5320 | LC CDR1 | RASQDVNTAVA |
| | | (Chothia) | |
| | 5321 | LC CDR2 | SASFLYS |
| | | (Chothia) | |
| | 5322 | LC CDR3 | QQHYTTPPT |
| | | (Chothia) | |
| | 5266 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5265 | DNA VL | gatatccagatgacccagtccccAagctccctgtcTgcctctgtgggGga |
| | | | tagggtcaccatcacctgcAGAgccagtcaggatgtgaatactgctgtag |
| | | | cctggtatcaacagaaaccaggaaaagctccCaaactactgatttactcT |
| | | | gcatccttcctcTACtctggagtcccttctAGAttctctggatccagatc |
| | | | tggCacTgaCttcactctgaccatcagTagtctgcagccTgaagacttTg |
| | | | caacttattactgtcaAcaacattatactactcctcccacCttIggacag |
| | | | ggtaccaaggtggagatcaaa |
| | 5351 | scFv | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSSGGGGSGGGGSGGGGSDIQMTQSPSSLSASV |
| | | | GDRVTITCRASQDVNTAVAWYQQKPGKAPKLLIYSASFLYSGVPSRFSGS |
| | | | RSGTDFTLTISSLQPEDFATYYCQQHYTTPPTFGQGTKVEIK |
| | 5364 | scFv DNA | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | ggggaTggcttctatgctatggacTACtggggtcaaggaaccctggtcac |
| | | | AgtctcctcTggtggtggtggatcTggaggaggaggaAGTggaggtggag |
| | | | gtTCTgatatccagatgacccagtccccAagctccctgtcTgcctctgtg |
| | | | ggGgatagggtcaccatcacctgcAGAgccagtcaggatgtgaatactgc |
| | | | tgtagcctggtatcaacagaaaccaggaaaagctccCaaactactgattt |
| | | | actcTgcatccttcctcTACtctggagtcccttctAGAttctctggatcc |
| | | | agatctggCacIgaCttcactctgaccatcagTagtctgcagccTgaaga |
| | | | cttTgcaacttattactgtcaAcaacattatactactcctcccacCttTg |
| | | | gacagggtaccaaggtggagatcaaa |
| | 5365 | Light Chain | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSSGGGGSGGGGSGGGGSDIQMTQSPSSLSASV |
| | | | GDRVTITCRASQDVNTAVAWYQQKPGKAPKLLIYSASFLYSGVPSRFSGS |
| | | | RSGTDFTLTISSLQPEDFATYYCQQHYTTPPTFGQGTKVEIKGSDIVLTQ |
| | | | SPDSLAVSLGERATINCRSSQTLLYSNNQKNYLAWYQKKPGQPPKLLISW |
| | | | AFTRKSGVPDRFSGSGSGTDFTLTISSLQAEDVAVYYCQQYSNYPWTFGQ |
| | | | GTKVEIKRTVAAPSVFIFPPSDEQLKSGTASVKCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5366 | DNA Light | gaggttcagctggtggagtctggTggtggcctGgtgcagccagggggctc |
| | | Chain | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | ggggaTggcttctatgctatggacTACtggggtcaaggaaccctggtcac |
| | | | AgtctcctcTggtggtggtggatcTggaggaggaggaAGTggaggtggag |
| | | | gtTCTgatatccagatgacccagtccccAagctccctgtcTgcctctgtg |
| | | | ggGgatagggtcaccatcacctgcAGAgccagtcaggatgtgaatactgc |
| | | | tgtagcctggtatcaacagaaaccaggaaaagctccCaaactactgattt |
| | | | actcTgcatccttcctcTACtctggagtcccttctAGAttctctggatcc |
| | | | agatctggCacTgaCttcactctgaccatcagTagtctgcagccTgaaga |
| | | | cttTgcaacttattactgtcaAcaacattatactactcctcccacCttTg |
| | | | gacagggtaccaaggtggagatcaaaGGCTCTgatatTGTACTGacccag |
| | | | tccccTGACtccctgGCTGTGtctCTGggGGAAaggGCAaccatcAACtg |
| | | | CAGGAGTagtcagACCCTTCTGTACTCTAATAACCAGAAAAACTACCTGg |
| | | | cctggtatcaaAAAaaaccaggaCAGCCTccTaaactactgattAGITGG |
| | | | gcaTTCACTAGGAAGtctggagtccctGACAGGttctctggatcTGGGtc |
| | | | TggAacAgatttcacActgaccatcagcagtctgcagGCTgaagaTGTTg |
| | | | caGTCtaCtactgtcagcaaTACTCAAACTACcctTGGacTttIggacag |
| | | | ggGaccaaAgtggagatcaaaAGGactgtCgctgcaccatctgtcttcat |
| | | | cttcccAccatctgatgagcagttgaaatctggaactgcctctgttAAGt |
| | | | gcctgctgaataacttctatcccagagaggccaaagtacagtggaaggtg |
| | | | gataaTgccctccaaAGTggtaactcccaggagagtgtcacagagcagga |
| | | | cagcaaggacagcacctacagcctcagcagcaccctgacCctgagcaaag |
| | | | cagactaTgagaaacacaaagtctaTgcctgTgaagtcacccatcagggc |
| | | | ctgagcAGCccAgtcacaaagagcttcaacaggggagagtgt |
| Ab-HER- | 5037 | HC CDR1 | DTYIH |
| 82 | | (Kabat) | |
| | 5038 | HC CDR2 | RIYPTNGYTRYADSVKG |
| | | (Kabat) | |
| | 5039 | HC CDR3 | WGGDGFYAMDY |
| | | (Kabat) | |
| | 5040 | HC CDR1 | GFNIKDT |
| | | (Chothia) | |
| | 5041 | HC CDR2 | YPTNGY |
| | | (Chothia) | |
| | 5042 | HC CDR3 | WGGDGFYAMDY |
| | | (Chothia) | |
| | 5361 | HC CDR1 | GFNIKDTYIH |
| | 5362 | HC CDR2 | RIYPTNGYTRYADSVKG |
| | 5363 | HC CDR3 | WGGDGFYAMDY |
| | 5361 | HC CDR1 | GFNIKDTYIH |
| | 5362 | HC CDR2 | RIYPTNGYTRYADSVKG |
| | 5363 | HC CDR3 | WGGDGFYAMDY |
| | 5001 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSS |
| | 5002 | DNA VH | gaggttcagctggtggagtctggcggtggcctggtgcagccagggggctc |
| | | | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcg |
| | 5368 | Heavy Chain | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAMDYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 5369 | DNA Heavy | gaggttcagctggtggagtctggcggtggcctggtgcagccagggggctc |
| | | Chain | actccgtttgtcctgtgcagcttctggcttcaacattaaagacacctata |
| | | | tacactgggtgcgtcaggccccgggtaagggcctggaatgggttgcaagg |
| | | | atttatcctacgaatggttatactagatatgccgatagcgtcaagggccg |
| | | | tttcactataagcgcagacacatccaaaaacacagcctacctgcagatga |
| | | | acagcctgcgtgctgaggacactgccgtctattattgttctagatgggga |
| | | | ggggacggcttctatgctatggacTACtggggtcaaggaaccctggtcac |
| | | | cgtctcctcggcgtcgaccaagggcccatcggtcttccccctggcaccct |
| | | | cctccaagagcacctctgggggcacagcggccctgggctgcctggtcaag |
| | | | gactacttccccgaacctgtgacggtctcgtggaactcaggcgccctgac |
| | | | cagcggcgtgcacaccttcccggctgtcctacagtcctcaggactctact |
| | | | ccctcagcagcgtggtgaccgtgccctccagcagcttgggcacccagacc |
| | | | tacatctgcaacgtgaatcacaagcccagcaacaccaaggtggacaagaG |
| | | | agttgagcccaaatcttgtgacaaaactcacacatgcccaccgtgcccag |
| | | | cacctgaactcGtggggggaccgtcagtcttcctcCtccccccaaaaccc |
| | | | aaggacaccctcatgatctcccggacccctgaggtcacatgcgtggtggt |
| | | | ggacgtgagccacgaagaccctgaggtcaagttcaactggtacgtggacg |
| | | | gcgtggaggtgcataatgccaagacaaagccgcCggaggagcagtacaac |
| | | | agcacgCTccgtgtggtcagcgtcctcaccgtcctgcaccaggactggct |
| | | | gaatggcaaggagtacaagtgcaaggtctccaacaaagccctcccagccc |
| | | | ccatcgagaaaaccatctccaaagccaaagggcagccccgagaaccacag |
| | | | gtgtacaccctgcccccatcccgggaTgagCtgaccaagaaccaggtcag |
| | | | cctgacctgcctggtcaaaggcttctatcccagcgacatcgccgtggagt |
| | | | gggagagcaatgggcagccggagaacaactacaagaccacgcctcTcgtg |
| | | | ctggactccgacggctccttcttcctctacagcaagctcaccgtggacaa |
| | | | gagcaggtggcagcaggggaacgtcttctcatgctccgtgatgcatgagg |
| | | | ctctgcacaaccactacacgcagaagagcctctccctgtctccgggtaaa |
| | 5317 | LC CDR1 | RASQDVNTAVA |
| | | (Kabat) | |
| | 5318 | LC CDR2 | SASFLYS |
| | | (Kabat) | |
| | 5319 | LC CDR3 | QQHYTTPPT |
| | | (Kabat) | |
| | 5320 | LC CDR1 | RASQDVNTAVA |
| | | (Chothia) | |
| | 5321 | LC CDR2 | SASFLYS |
| | | (Chothia) | |
| | 5322 | LC CDR3 | QQHYTTPPT |
| | | (Chothia) | |
| | 5006 | VL | DIQMTQSPSSLSASVGDRVTITCRASQDVNTAVAWYQQKPGKAPKLLIYS |
| | | | ASFLYSGVPSRFSGSRSGTDFTLTISSLQPEDFATYYCQQHYTTPPTFGQ |
| | | | GTKVEIK |
| | 5005 | DNA VL | gatatccagatgacccagtccccgagctccctgtccgcctctgtgggcga |
| | | | tagggtcaccatcacctgccgtgccagtcaggatgtgaatactgctgtag |
| | | | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaa |
| | 5036 | Light Chain | DIQMTQSPSSLSASVGDRVTITCRASQDVNTAVAWYQQKPGKAPKLLIYS |
| | | (hIgG kappa) | ASFLYSGVPSRFSGSRSGTDFTLTISSLQPEDFATYYCQQHYTTPPTFGQ |
| | | | GTKVEIKRTVAAPSVFIFPPSDEQLKSGTASVVCLLNNFYPREAKVQWKV |
| | | | DNALQSGNSQESVTEQDSKDSTYSLSSTLTLSKADYEKHKVYACEVTHQG |
| | | | LSSPVTKSENRGEC |
| | 5035 | DNA Light | gatatccagatgacccagtccccgagctccctgtccgcctctgtgggcga |
| | | Chain (hIgG | tagggtcaccatcacctgccgtgccagtcaggatgtgaatactgctgtag |
| | | kappa) | cctggtatcaacagaaaccaggaaaagctccgaaactactgatttactcg |
| | | | gcatccttcctcTACtctggagtcccttctcgcttctctggatccagatc |
| | | | tgggacggatttcactctgaccatcagcagtctgcagccggaagacttcg |
| | | | caacttattactgtcagcaacattatactactcctcccacgttcggacag |
| | | | ggtaccaaggtggagatcaaacgaactgtggctgcaccatctgtcttcat |
| | | | cttcccgccatctgatgagcagttgaaatctggaactgcctctgttgtgt |
| | | | gcctgctgaataacttctatcccagagaggccaaagtacagtggaaggtg |
| | | | gataacgccctccaatcgggtaactcccaggagagtgtcacagagcagga |
| | | | cagcaaggacagcacctacagcctcagcagcaccctgacgctgagcaaag |
| | | | cagactacgagaaacacaaagtctacgcctgcgaagtcacccatcagggc |
| | | | ctgagctcgcccgtcacaaagagcttcaacaggggagagtgt |
| | 5370 | DARPIN | GSDLGKKLLEAARAGQDDEVRILMANGADVNAHDFYGITPLHLAANFGHL |
| | | | EIVEVLLKHGADVNAFDYDNTPLHLAADAGHLEIVEVLLKYGADVNASDR |
| | | | DGHTPLHLAAREGHLEIVEVLLKNGADVNAQDKFGKTAFDISIDNGNEDL |
| | | | AEILQKL |
| | 5371 | DARPIN | GGCTCTGATCTGGGAAAAAAGCTTCTgGAGGCtGCcAGAGCtGGACAGGA |
| | | DNA | tGAtGAAGTGagaATACTGATGGCTAAtGGTGCTGATGTGAATGCCCAtG |
| | | | ATTTTTATGGAATCACTCCCTTACACTTAGCTGCCAATTTtGGGCACCTg |
| | | | GAGATTGTGGAAGTGCTCCTGAAGCATGGaGCAGATGTAAAtGCATTtGA |
| | | | TTAtGATAACACTCCACTGCACCTGGCtGCAGATGCtGGACATCTGGAGA |
| | | | TtGTGGAAGTACTGTTAAAATAGGAGCAGAtGTGAATGCATCtGACaga |
| | | | GATGGACATACACCACTGCACCTGGCAGCTagaGAGGGACATCTTGAAAT |
| | | | TGTTGAAGTCCTGCTTAAGAAtGGTGCtGATGTCAATGCCCAGGACAAGT |
| | | | TTGGGAAGACAGCTTTTGACATTTCAATtGATAAtGGAAAtGAGGATCTG |
| | | | GCtGAAATACTTCAGAAATTG |
| | 5372 | Light Chain | GSDLGKKLLEAARAGQDDEVRILMANGADVNAHDFYGITPLHLAANFGHL |
| | | | EIVEVLLKHGADVNAFDYDNTPLHLAADAGHLEIVEVLLKYGADVNASDR |
| | | | DGHTPLHLAAREGHLEIVEVLLKNGADVNAQDKFGKTAFDISIDNGNEDL |
| | | | AEILQKLGGGGSGGGGSGGGGSDIQMTQSPSSLSASVGDRVTITCRASQD |
| | | | VNTAVAWYQQKPGKAPKLLIYSASFLYSGVPSRESGSRSGTDFTLTISSL |
| | | | QPEDFATYYCQQHYTTPPTFGQGTKVEIKRTVAAPSVFIFPPSDEQLKSG |
| | | | TASVVCLLNNFYPREAKVQWKVDNALQSGNSQESVTEQDSKDSTYSLSST |
| | | | LTLSKADYEKHKVYACEVTHQGLSSPVTKSFNRGEC |
| | 5373 | Light Chain | GGCTCTGATCTGGGAAAAAAGCTTCTgGAGGCtGCcAGAGCtGGACAGGA |
| | | DNA | tGAtGAAGTGagaATACTGATGGCTAAtGGTGCTGATGTGAATGCCCAtG |
| | | | ATTTTTATGGAATCACTCCCTTACACTTAGCTGCCAATTItGGGCACCTg |
| | | | GAGATTGTGGAAGTGCTCCTGAAGCATGGaGCAGATGTAAAtGCATTtGA |
| | | | TTAtGATAACACTCCACTGCACCTGGCtGCAGATGCtGGACATCTGGAGA |
| | | | TtGTGGAAGTACTGTTAAAATAtGGAGCAGAtGTGAATGCATCtGACaga |
| | | | GATGGACATACACCACTGCACCTGGCAGCTagaGAGGGACATCTTGAAAT |
| | | | TGTTGAAGTCCTGCTTAAGAAtGGTGCtGATGTCAATGCCCAGGACAAGT |
| | | | TTGGGAAGACAGCTTTTGACATTTCAATtGATAAtGGAAAtGAGGATCTG |
| | | | GCtGAAATACTICAGAAATTGggtggtggtggatctggaggaggaggaAG |
| | | | TggaggtggaggtTCTgatatccagatgacccagtccccgagctccctgt |
| | | | ccgcctctgtgggcgatagggtcaccatcacctgccgtgccagtcaggat |
| | | | gtgaatactgctgtagcctggtatcaacagaaaccaggaaaagctccgaa |
| | | | actactgatttactcggcatccttcctcTACtctggagtcccttctcgct |
| | | | tctctggatccagatctgggacggatttcactctgaccatcagcagtctg |
| | | | cagccggaagacttcgcaacttattactgtcagcaacattatactactcc |
| | | | tcccacgttcggacagggtaccaaggtggagatcaaacgaactgtggctg |
| | | | caccatctgtcttcatcttcccgccatctgatgagcagttgaaatctgga |
| | | | actgcctctgttgtgtgcctgctgaataacttctatcccagagaggccaa |
| | | | agtacagtggaaggtggataacgccctccaatcgggtaactcccaggaga |
| | | | gtgtcacagagcaggacagcaaggacagcacctacagcctcagcagcacc |
| | | | ctgacgctgagcaaagcagactacgagaaacacaaagtctacgcctgcga |
| | | | agtcacccatcagggcctgagctcgcccgtcacaaagagcttcaacaggg |
| | | | gagagtgt |

TABLE 3B
Exemplary Anti-HER2 Antibody Variants Based on HER-75 with Heavy Chain
Mutations

| | SEQ | | |
|---|---|---|---|
| Ab ID | ID NO | Description | Sequence |
| Ab-HER- | 6510 | HC CDR3 | WGGWGFYAFAL |
| 88 | | (Kabat) | |
| | 6511 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAFALWGQGTLVTVSS |
| | 6512 | DNA VH | gaggttcagctggtggagtctggAggtggcctggtgcagccagggggctc |
| | | | actcAGAttgtcctgtgcagcttctggcttcaacattaaagacacctata |
| | | | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctTTTGCCCTGtggggtcaaggaaccctggtcac |
| | | | AgtctcctcT |
| | 6513 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GWGFYAFALWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 6514 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctTTTGCCCTGtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccTgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatIgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |
| Ab-HER- | 6515 | HC CDR3 | WGGWGFYAFAY |
| 89 | | (Kabat) | |
| | 6516 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAFAYWGQGTLVTVSS |
| | 6517 | DNA VH | gaggttcagctggtggagtctggAggtggcctggtgcagccagggggctc |
| | | | actcAGAttgtcctgtgcagcttctggcttcaacattaaagacacctata |
| | | | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctTTTGCCTACtggggtcaaggaaccctggtcac |
| | | | AgtctcctcT |
| | 6518 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GWGFYAFAYWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 6519 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctTTTGCCTACtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccTgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatTgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |
| Ab-HER- | 6520 | HC CDR3 | WGGWGFYAFDL |
| 90 | | (Kabat) | |
| | 6521 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAFDLWGQGTLVTVSS |
| | 6522 | DNA VH | gaggttcagctggtggagtctggAggtggcctggtgcagccagggggctc |
| | | | actcAGAttgtcctgtgcagcttctggcttcaacattaaagacacctata |
| | | | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctTTTGACCTGtggggtcaaggaaccctggtcac |
| | | | AgtctcctcT |
| | 6523 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GWGFYAFDLWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 6524 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcIgtctattattgttctagatgggga |
| | | | gggTGGggcttctatgctTTTGACCTGtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccIgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatIgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |
| Ab-HER- | 6525 | HC CDR3 | WGGWGFYAMAL |
| 91 | | (Kabat) | |
| | 6526 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAMALWGQGTLVTVSS |
| | 6527 | DNA VH | gaggttcagctggtggagtctggAggtggcctggtgcagccagggggctc |
| | | | actcAGAttgtcctgtgcagcttctggcttcaacattaaagacacctata |
| | | | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctATGGCCCTGtggggtcaaggaaccctggtcac |
| | | | AgtctcctcT |
| | 6528 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GWGFYAMALWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 6529 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggTGGggcttctatgctATGGCCCTGtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccIgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatIgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |
| Ab-HER- | 6530 | HC CDR3 | WGGDGFYAFAL |
| 92 | | (Kabat) | |
| | 6531 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAFALWGQGTLVTVSS |
| | 6532 | DNA VH | gaggttcagctggtggagtctggAggtggcctggtgcagccagggggctc |
| | | | actcAGAttgtcctgtgcagcttctggcttcaacattaaagacacctata |
| | | | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggGATggcttctatgctTTTGCCCTGtggggtcaaggaaccctggtcac |
| | | | AgtctcctcT |
| | 6533 | Heavy Chain | EVQLVESGGGLVQPGGSLRLSCAASGFNIKDTYIHWVRQAPGKGLEWVAR |
| | | (hIgG1) | IYPTNGYTRYADSVKGRFTISADTSKNTAYLQMNSLRAEDTAVYYCSRWG |
| | | | GDGFYAFALWGQGTLVTVSSASTKGPSVFPLAPSSKSTSGGTAALGCLVK |
| | | | DYFPEPVTVSWNSGALTSGVHTFPAVLQSSGLYSLSSVVTVPSSSLGTQT |
| | | | YICNVNHKPSNTKVDKRVEPKSCDKTHTCPPCPAPELVGGPSVFLLPPKP |
| | | | KDTLMISRTPEVTCVVVDVSHEDPEVKFNWYVDGVEVHNAKTKPPEEQYN |
| | | | STLRVVSVLTVLHQDWLNGKEYKCKVSNKALPAPIEKTISKAKGQPREPQ |
| | | | VYTLPPSRDELTKNQVSLTCLVKGFYPSDIAVEWESNGQPENNYKTTPLV |
| | | | LDSDGSFFLYSKLTVDKSRWQQGNVFSCSVMHEALHNHYTQKSLSLSPGK |
| | 6534 | DNA Heavy | gaggttcagctggtggagtctggAggtggcctggtgcagccagggggctc |
| | | Chain | actcAGAttgtcctgtgcagcttctggcttcaacattaaagacacctata |
| | | (hIgG1) | tacactgggtgagacaggccccAggtaagggcctggaatgggttgcaagg |
| | | | atttatcctacCaatggttatactagatatgcTgatTCTgtcaagggcAG |
| | | | AttcactataTCTgcagacacatccaaaaacacagcctacctgcagatga |
| | | | acagcctgAGAgctgaggacactgcTgtctattattgttctagatgggga |
| | | | gggGATggcttctatgctTTTGCCCTGtggggtcaaggaaccctggtcac |
| | | | AgtctcctcTgcCtcTaccaagggcccatcTgtcttccccctggcaccct |
| | | | cctccaagagcacctctgggggcacagcTgccctgggctgcctggtcaag |
| | | | gactacttcccTgaacctgtgacAgtctcCtggaactcaggAgccctgac |
| | | | cagTggAgtgcacaccttcccTgctgtcctacagtcctcaggactctact |
| | | | ccctcagcTCTgtggtgacAgtgccctccagcagcttgggcacccagacc |
| | | | tacatctgcaaTgtgaatcacaagcccagcaacaccaaggtggacaagaG |
| | | | agttgagcccaaatcttgtgacaaaactcacacatgcccaccTtgcccag |
| | | | cacctgaactGGtggggggaccAtcagtcttcctcCtccccccaaaaccc |
| | | | aaggacaccctcatgatctccAGAacccctgaggtcacatgTgtggtggt |
| | | | ggaTgtgagccaTgaagaccctgaggtcaagttcaactggtaTgtggaTg |
| | | | gAgtggaggtgcataatgccaagacaaagccCcCTgaggagcagtacaac |
| | | | agcacTCTcAGAgtggtcTCTgtcctcacAgtcctgcaccaggactggct |
| | | | gaatggcaaggagtacaagtgcaaggtctccaacaaagccctcccagccc |
| | | | ccatTgagaaaaccatctccaaagccaaagggcagcccAGAgaaccacag |
| | | | gtgtacaccctgcccccatccAGAgaTgagCtgaccaagaaccaggtcag |
| | | | cctgacctgcctggtcaaaggcttctatcccTCTgacatIgcTgtggagt |
| | | | gggagagcaatgggcagccTgagaacaactacaagaccacAcctcTTgtg |
| | | | ctggactcTgaTggctccttcttcctctacagcaagctcacAgtggacaa |
| | | | gagcaggtggcagcaggggaaTgtcttctcatgcTCTgtgatgcatgagg |
| | | | ctctgcacaaccactacacCcagaagagcctctccctgtctccAggtaaa |

TABLE 3C
Exemplary Anti-HER2 Antibody Variants Based on HER-78 with Heavy Chain
Mutations

| | SEQ | | |
|---|---|---|---|
| Ab ID | ID NO | Description | Sequence |
| Ab-HER- | 6535 | HC CDR3 | WGGWGFYAFAL |
| 97 | | (Kabat) | |
| | 6536 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAFALWGQGTLVTVSS |
| | 6537 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | gggTGGggcttctatgctTTTGCCCTGtggggtcaaggaaccctggtcac |
| | | | AgtctcctcT |
| Ab-HER- | 6538 | HC CDR3 | WGGWGFYAFAY |
| 98 | | (Kabat) | |
| | 6539 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAFAYWGQGTLVTVSS |
| | 6540 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | gggTGGggcttctatgctTTTGCCTACtggggtcaaggaaccctggtcac |
| | | | AgtctcctcT |
| Ab-HER- | 6541 | HC CDR3 | WGGWGFYAFDL |
| 99 | | (Kabat) | |
| | 6542 | VH | EVQLVESGGGLVQPGGSLRLSCAASGENIKDTYIHWVRQAPGKGLEWVAR |
| | | | IYPTNGYTRYADSVKGRFTISADTSKNTAYLQMNSLRAEDTAVYYCSRWG |
| | | | GWGFYAFDLWGQGTLVTVSS |
| | 6543 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccTggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | gggTGGggcttctatgctTTTgacCTGtggggtcaaggaaccctggtcac |
| | | | AgtctcctcT |
| Ab-HER- | 6544 | HC CDR3 | WGGWGFYAMAL |
| 100 | | (Kabat) | |
| | 6545 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GWGFYAMALWGQGTLVTVSS |
| | 6546 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccIggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | gggTGGggcttctatgctATGGCCCTGtggggtcaaggaaccctggtcac |
| | | | AgtctcctcT |
| Ab-HER- | 6547 | HC CDR3 | WGGDGFYAFAL |
| 101 | | (Kabat) | |
| | 6548 | VH | EVQLVESGGGLVQPGGSLRLSCAASGFNIKDTYIHWVRQAPGKGLEWVAR |
| | | | IYPTNGYTRYADSVKGRFTISADTSKNTAYLQMNSLRAEDTAVYYCSRWG |
| | | | GDGFYAFALWGQGTLVTVSS |
| | 6549 | DNA VH | gaggttcagctggtggagtctggTggtggcctGgtgcagccagggggctc |
| | | | actcAGAttgtcctgtgcagcttctggcttcaacattaaagacacctata |
| | | | tacactgggtgAGAcaggccccIggAaagggcctggaatgggttgcaagg |
| | | | atttatcctacTaatggttatactagatatgcAgatagTgtcaagggcAG |
| | | | AttcactataagTgcagacacatccaaaaacacagcctacctgcagatga |
| | | | acagcctgAGAgctgaggacactgcAgtctattattgttctagatgggga |
| | | | gggGATggcttctatgctTTTGCCCTGtggggtcaaggaaccctggtcac |
| | | | AgtctcctcT |

TABLE 20
Constant Regions of Heavy Chains and Light Chains

| SEQ | | |
|---|---|---|
| ID | | |
| NO | Description | Sequence |
| 5003 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaa |
| | | agttgagcccCCCaaatcttgtgacaaaactcacacatgcccaccgtgcccagcacct |
| | | gaactcctggggggaccgtcagtcttcctcttccccccaaaacccaaggacaccctca |
| | | tgatctcccggacccctgaggtcacatgcgtggtggtggacgtgagccacgaagaccc |
| | | tgaggtcaagttcaactggtacgtggacggcgtggaggtgcataatgccaagacaaag |
| | | ccgcgggaggagcagtacaacagcacgtaccgtgtggtcagcgtcctcaccgtcctgc |
| | | accaggactggctgaatggcaaggagtacaagtgcaaggtctccaacaaagccctccc |
| | | agcccccatcgagaaaaccatctccaaagccaaagggcagccccgagaaccacaggtg |
| | | tacaccctgcccccatcccgggatgagctgaccaagaaccaggtcagcctgacctgcc |
| | | tggtcaaaggcttctatcccagcgacatcgccgtggagtgggagagcaatgggcagcc |
| | | ggagaacaactacaagaccacgcctcccgtgctggactccgacggctccttcttcctc |
| | | tacagcaagctcaccgtggacaagagcaggtggcagcaggggaacgtcttctcatgct |
| | | ccgtgatgcatgaggctctgcacaaccactacacgcagaagagcctctccctgtctcc |
| | | gggtaaa |
| 5004 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPPKSCDKTHTCPPCPAP |
| | Heavy Chain | ELLGGPSVFLFPPKPKDTLMISRTPEVTCVVVDVSHEDPEVKFNWYVDGVEVHNAKTK |
| | (hIgG1) | PREEQYNSTYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 5007 | DNA Constant | cgaactgtggctgcaccatctgtcttcatcttcccgccatctgatgagcagttgaaat |
| | Region of Light | ctggaactgcctctgttgtgtgcctgctgaataacttctatcccagagaggccaaagt |
| | Chain (hIgG1 | acagtggaaggtggataacgccctccaatcgggtaactcccaggagagtgtcacagag |
| | kappa) | caggacagcaaggacagcacctacagcctcagcagcaccctgacgctgagcaaagcag |
| | | actacgagaaacacaaagtctacgcctgcgaagtcacccatcagggcctgagctcgcc |
| | | cgtcacaaagagcttcaacaggggagagtgt |
| 5008 | Constant | RTVAAPSVFIFPPSDEQLKSGTASVVCLLNNFYPREAKVQWKVDNALQSGNSQESVTE |
| | Region of Light | QDSKDSTYSLSSTLTLSKADYEKHKVYACEVTHQGLSSPVTKSENRGEC |
| | Chain (hIgG | |
| | Kappa) | |
| 5017 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaG |
| | | agttgagcccaaatcttgtgacaaaactcacacatgcccaccgtgcccagcacctgaa |
| | | ctcGtggggggaccgtcagtcttcctcCtccccccaaaacccaaggacaccctcatga |
| | | tctcccggacccctgaggtcacatgcgtggtggtggacgtgagccacgaagaccctga |
| | | ggtcaagttcaactggtacgtggacggcgtggaggtgcataatgccaagacaaagccg |
| | | cCggaggagcagtacaacagcacgCTccgtgtggtcagcgtcctcaccgtcctgcacc |
| | | aggactggctgaatggcaaggagtacaagtgcaaggtctccaacaaagccctcccagc |
| | | ccccatcgagaaaaccatctccaaagccaaagggcagccccgagaaccacaggtgtac |
| | | accctgcccccatcccgggaTgagCtgaccaagaaccaggtcagcctgacctgcctgg |
| | | tcaaaggcttctatcccagcgacatcgccgtggagtgggagagcaatgggcagccgga |
| | | gaacaactacaagaccacgcctcTcgtgctggactccgacggctccttcttcctctac |
| | | agcaagctcaccgtggacaagagcaggtggcagcaggggaacgtcttctcatgctccg |
| | | tgatgcatgaggctctgcacaaccactacacgcagaagagcctctccctgtctccggg |
| | | taaa |
| 5018 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKRVEPKSCDKTHTCPPCPAPE |
| | Heavy Chain | LVGGPSVFLLPPKPKDTLMISRTPEVTCVVVDVSHEDPEVKFNWYVDGVEVHNAKTKP |
| | (hIgG1) | PEEQYNSTLRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPLVLDSDGSFFLY |
| | | SKLTVDKSRWQQGNVFSCSVMHEALHNHYTQKSLSLSPGK |
| 5021 | DNA Constant | AGAACCGTGGCCGCCCCTAGCGTGTTCATCTTCCCGCCAAGCGACGAGCAGCTGAAGA |
| | Region of Light | GCGGCACCGCCAGCGTGGTGTGCCTGCTGAACAACTTCTACCCCAGAGAGGCCAAGGT |
| | Chain (hIgG1 | GCAGTGGAAGGTGGACAACGCCCTGCAGAGCGGCAACAGCCAGGAGAGCGTGACCGAG |
| | kappa) | CAGGACAGCAAGGACAGCACCTACAGCCTGAGCAGCACCCTGACCCTGAGCAAGGCCG |
| | | ACTACGAGAAGCACAAGGTGTACGCCTGCGAGGTGACCCACCAGGGCCTGAGCAGCCC |
| | | CGTGACCAAGAGCTTCAACAGAGGCGAGTGCt |
| 5022 | Constant | RTVAAPSVFIFPPSDEQLKSGTASVVCLLNNFYPREAKVQWKVDNALQSGNSQESVTE |
| | Region of Light | QDSKDSTYSLSSTLTLSKADYEKHKVYACEVTHQGLSSPVTKSENRGEC |
| | Chain (hIgG | |
| | Kappa) | |
| 5011 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaa |
| | | agttgagcccaaatcttgtgacaaaactcacacatgcccaccgtgcccagcacctgaa |
| | | ctcctggggggaccgtcagtcttcctcttccccccaaaacccaaggacaccctcatga |
| | | tctcccggacccctgaggtcacatgcgtggtggtggacgtgagccacgaagaccctga |
| | | ggtcaagttcaactggtacgtggacggcgtggaggtgcataatgccaagacaaagccg |
| | | cgggaggagcagtacaacagcacgtaccgtgtggtcagcgtcctcaccgtcctgcacc |
| | | aggactggctgaatggcaaggagtacaagtgcaaggtctccaacaaagccctcccagc |
| | | ccccatcgagaaaaccatctccaaagccaaagggcagccccgagaaccacaggtgtac |
| | | accctgcccccatcccgggaGgagAtgaccaagaaccaggtcagcctgacctgcctgg |
| | | tcaaaggcttctatcccagcgacatcgccgtggagtgggagagcaatgggcagccgga |
| | | gaacaactacaagaccacgcctcccgtgctggactccgacggctccttcttcctctac |
| | | agcaagctcaccgtggacaagagcaggtggcagcaggggaacgtcttctcatgctccg |
| | | tgatgcatgaggctctgcacaaccactacacgcagaagagcctctccctgtctccggg |
| | | taaa |
| 5012 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPKSCDKTHTCPPCPAPE |
| | Heavy Chain | LLGGPSVFLFPPKPKDTLMISRTPEVTCVVVDVSHEDPEVKENWYVDGVEVHNAKTKP |
| | (hIgG1) | REEQYNSTYRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 5025 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaa |
| | | agttgagcccCCCaaatcttgtgacaaaactcacacatgcccaccgtgcccagcacct |
| | | gaactcctggggggaccgtcagtcttcctcttccccccaaaacccaaggacaccctca |
| | | tgatctcccggacccctgaggtcacatgcgtggtggtggacgtgagccacgaagaccc |
| | | tgaggtcaagttcaactggtacgtggacggcgtggaggtgcataatgccaagacaaag |
| | | ccgcgggaggagcagtacaacagcacgtaccgtgtggtcagcgtcctcaccgtcctgc |
| | | accaggactggctgaatggcaaggagtacaagtgcaaggtctccaacaaagccctccc |
| | | agcccccatcgagaaaaccatctccaaagccaaagggcagccccgagaaccacaggtg |
| | | tacaccctgcccccatcccgggatgagctgaccaagaaccaggtcagcctgacctgcc |
| | | tggtcaaaggcttctatcccagcgacatcgccgtggagtgggagagcaatgggcagcc |
| | | ggagaacaactacaagaccacgcctcccgtgctggactccgacggctccttcttcctc |
| | | tacagcaagctcaccgtggacaagagcaggtggcagcaggggaacgtcttctcatgct |
| | | ccgtgatgcatgaggctctgcacaacCAGtacacgcagaagagcctctccctgtctcc |
| | | gggtaaa |
| 5026 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPPKSCDKTHTCPPCPAP |
| | Heavy Chain | ELLGGPSVFLFPPKPKDTLMISRTPEVTCVVVDVSHEDPEVKFNWYVDGVEVHNAKTK |
| | (hIgG1) | PREEQYNSTYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFL |
| | | YSKLTVDKSRWQQGNVFSCSVMHEALHNQYTQKSLSLSPGK |
| 5029 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaa |
| | | agttgagcccCCCaaatcttgtgacaaaactcacacatgcccaccgtgcccagcacct |
| | | gaactcctggggggaccgtcagtcttcctcttccccccaaaacccaaggacaccctca |
| | | tgGCAtcccggacccctgaggtcacatgcgtggtggtggacgtgagccacgaagaccc |
| | | tgaggtcaagttcaactggtacgtggacggcgtggaggtgcataatgccaagacaaag |
| | | ccgcgggaggagcagtacaacagcacgtaccgtgtggtcagcgtcctcaccgtcctgG |
| | | CCcaggactggctgaatggcaaggagtacaagtgcaaggtctccaacaaagccctccc |
| | | agcccccatcgagaaaaccatctccaaagccaaagggcagccccgagaaccacaggtg |
| | | tacaccctgcccccatcccgggatgagctgaccaagaaccaggtcagcctgacctgcc |
| | | tggtcaaaggcttctatcccagcgacatcgccgtggagtgggagagcaatgggcagcc |
| | | ggagaacaactacaagaccacgcctcccgtgctggactccgacggctccttcttcctc |
| | | tacagcaagctcaccgtggacaagagcaggtggcagcaggggaacgtcttctcatgct |
| | | ccgtgatgcatgaggctctgcacaacGCTtacacgcagaagagcctctccctgtctcc |
| | | gggtaaa |
| 5030 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPPKSCDKTHTCPPCPAP |
| | Heavy Chain | ELLGGPSVFLFPPKPKDTLMASRTPEVTCVVVDVSHEDPEVKFNWYVDGVEVHNAKTK |
| | (hIgG1) | PREEQYNSTYRVVSVLTVLAQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFL |
| | | YSKLTVDKSRWQQGNVFSCSVMHEALHNAYTQKSLSLSPGK |
| 5211 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaa |
| | | agttgagcccCCCaaatcttgtgacaaaactcacacatgcccaccgtgcccagcacct |
| | | gaactcctggggggaccgtcagtcttcctcttccccccaaaacccaaggacaccctca |
| | | tgGCAtcccggacccctgaggtcacatgcgtggtggtggacgtgagccacgaagaccc |
| | | tgaggtcaagttcaactggtacgtggacggcgtggaggtgcataatgccaagacaaag |
| | | ccgcgggaggagcagtacaacagcacgtaccgtgtggtcagcgtcctcaccgtcctgG |
| | | CCcaggactggctgaatggcaaggagtacaagtgcaaggtctccaacaaagccctccc |
| | | agcccccatcgagaaaaccatctccaaagccaaagggcagccccgagaaccacaggtg |
| | | tacaccctgcccccatcccgggatgagctgaccaagaaccaggtcagcctgacctgcc |
| | | tggtcaaaggcttctatcccagcgacatcgccgtggagtgggagagcaatgggcagcc |
| | | ggagaacaactacaagaccacgcctcccgtgctggactccgacggctccttcttcctc |
| | | tacagcaagctcaccgtggacaagagcaggtggcagcaggggaacgtcttctcatgct |
| | | ccgtgatgcatgaggctctgcacaaccactacacgcagaagagcctctccctgtctcc |
| | | gggtaaa |
| 5212 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPPKSCDKTHTCPPCPAP |
| | Heavy Chain | ELLGGPSVFLFPPKPKDTLMASRTPEVTCVVVDVSHEDPEVKENWYVDGVEVHNAKTK |
| | (hIgG1) | PREEQYNSTYRVVSVLTVLAQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 5213 | DNA Constant | gcgtcgaccaagggcccatcggtcttccccctggcaccctcctccaagagcacctctg |
| | Region of | ggggcacagcggccctgggctgcctggtcaaggactacttccccgaacctgtgacggt |
| | Heavy Chain | ctcgtggaactcaggcgccctgaccagcggcgtgcacaccttcccggctgtcctacag |
| | (hIgG1) | tcctcaggactctactccctcagcagcgtggtgaccgtgccctccagcagcttgggca |
| | | cccagacctacatctgcaacgtgaatcacaagcccagcaacaccaaggtggacaagaa |
| | | agttgagcccCCCaaatcttgtgacaaaactcacacatgcccaccgtgcccagcacct |
| | | gaactcctggggggaccgtcagtcttcctcttccccccaaaacccaaggacaccctca |
| | | tgGCAtcccggacccctgaggtcacatgcgtggtggtggacgtgagccacgaagaccc |
| | | tgaggtcaagttcaactggtacgtggacggcgtggaggtgcataatgccaagacaaag |
| | | ccgcgggaggagcagtacaacagcacgtaccgtgtggtcagcgtcctcaccgtcctgc |
| | | accaggactggctgaatggcaaggagtacaagtgcaaggtctccaacaaagccctccc |
| | | agcccccatcgagaaaaccatctccaaagccaaagggcagccccgagaaccacaggtg |
| | | tacaccctgcccccatcccgggatgagctgaccaagaaccaggtcagcctgacctgcc |
| | | tggtcaaaggcttctatcccagcgacatcgccgtggagtgggagagcaatgggcagcc |
| | | ggagaacaactacaagaccacgcctcccgtgctggactccgacggctccttcttcctc |
| | | tacagcaagctcaccgtggacaagagcaggtggcagcaggggaacgtcttctcatgct |
| | | ccgtgatgcatgaggctctgcacaacCAGtacacgcagaagagcctctccctgtctcc |
| | | gggtaaa |
| 5214 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPPKSCDKTHTCPPCPAP |
| | Heavy Chain | ELLGGPSVFLFPPKPKDTLMASRTPEVTCVVVDVSHEDPEVKFNWYVDGVEVHNAKTK |
| | (hIgG1) | PREEQYNSTYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFL |
| | | YSKLTVDKSRWQQGNVFSCSVMHEALHNQYTQKSLSLSPGK |
| 5215 | DNA Constant | gcCtcTaccaagggcccatcTgtcttccccctggcaccctcctccaagagcacctctg |
| | Region of | ggggcacagcAgccctgggctgcctggtcaaggactacttcccTgaacctgtgacAgt |
| | Heavy Chain | ctcTtggaactcaggGgccctgaccTCTggGgtgcacaccttcccAgctgtcctacag |
| | (hIgG1) | tcctcaggactctactccctcTCTTCTgtggtgacAgtgccctccagcagcttgggca |
| | | cccagacctacatctgcaaTgtgaatcacaagcccagcaacaccaaggtggacaagaa |
| | | agttgagcccCCCaaatcttgtgacaaaactcacacatgcccaccCtgcccagcacct |
| | | gaactcctggggggaccCtcagtcttcctcttccccccaaaacccaaggacaccctca |
| | | tgatctccAGAacccctgaggtcacatgIgtggtggtggaTgtgagccaTgaagaccc |
| | | tgaggtcaagttcaactggtaTgtggaTggGgtggaggtgcataatgccaagacaaag |
| | | ccCAGAgaggagcagtacaacagcacCtacAGAgtggtcTCAgtcctcacTgtcctgc |
| | | accaggactggctgaatggcaaggagtacaagtgcaaggtctccaacaaagccctccc |
| | | agcccccatAgagaaaaccatctccaaagccaaagggcagcccAGAgaaccacaggtg |
| | | tacaccctgcccccatccAGAgatgagctgaccaagaaccaggtcagcctgacctgcc |
| | | tggtcaaaggcttctatcccTCTgacatIgcTgtggagtgggagagcaatgggcagcc |
| | | AgagaacaactacaagaccacCcctccAgtgctggactcTgatggctccttcttcctc |
| | | tacagcaagctcacAgtggacaagagcaggtggcagcaggggaaTgtcttctcatgct |
| | | cTgtgatgcatgaggctctgcacaaccactacacTcagaagagcctctccctgtctcc |
| | | Tggtaaa |
| 5216 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPPKSCDKTHTCPPCPAP |
| | Heavy Chain | ELLGGPSVFLFPPKPKDTLMISRTPEVTCVVVDVSHEDPEVKFNWYVDGVEVHNAKTK |
| | (hIgG1) | PREEQYNSTYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 5217 | DNA Constant | AGGactgtCgctgcaccatctgtcttcatcttcccAccatctgatgagcagttgaaat |
| | Region of Light | ctggaactgcctctgttAAGtgcctgctgaataacttctatcccagagaggccaaagt |
| | Chain | acagtggaaggtggataaTgccctccaaAGTggtaactcccaggagagtgtcacagag |
| | | caggacagcaaggacagcacctacagcctcagcagcaccctgacCctgagcaaagcag |
| | | actaTgagaaacacaaagtctaTgcctgTgaagtcacccatcagggcctgagcAGCcc |
| | | Agtcacaaagagcttcaacaggggagagtgt |
| 5218 | Constant | RTVAAPSVFIFPPSDEQLKSGTASVKCLLNNFYPREAKVQWKVDNALQSGNSQESVTE |
| | Region of Light | QDSKDSTYSLSSTLTLSKADYEKHKVYACEVTHQGLSSPVTKSENRGEC |
| | Chain | |
| 5219 | DNA Constant | gcCtcTaccaagggcccatcTgtcttccccctggcaccctcctccaagagcacctctg |
| | Region of | ggggcacagcTgccctgggctgcctggtcaaggactacttcccTgaacctgtgacAgt |
| | Heavy Chain | ctcCtggaactcaggAgccctgaccagTggAgtgcacaccttcccTgctgtcctacag |
| | (hIgG1) | tcctcaggactctactccctcagcTCTgtggtgacAgtgccctccagcagcttgggca |
| | | cccagacctacatctgcaaTgtgaatcacaagcccagcaacaccaaggtggacaagaG |
| | | agttgagcccaaatcttgtgacaaaactcacacatgcccaccTtgcccagcacctgaa |
| | | ctGGtggggggaccAtcagtcttcctcCtccccccaaaacccaaggacaccctcatga |
| | | tctccAGAacccctgaggtcacatgTgtggtggtggaTgtgagccaTgaagaccctga |
| | | ggtcaagttcaactggtaTgtggaTggAgtggaggtgcataatgccaagacaaagccC |
| | | cCTgaggagcagtacaacagcacTCTcAGAgtggtcTCTgtcctcacAgtcctgcacc |
| | | aggactggctgaatggcaaggagtacaagtgcaaggtctccaacaaagccctcccagc |
| | | ccccatTgagaaaaccatctccaaagccaaagggcagcccAGAgaaccacaggtgtac |
| | | accctgcccccatccAGAgaTgagCtgaccaagaaccaggtcagcctgacctgcctgg |
| | | tcaaaggcttctatcccTCTgacatTgcTgtggagtgggagagcaatgggcagccTga |
| | | gaacaactacaagaccacAcctcTTgtgctggactcTgaTggctccttcttcctctac |
| | | agcaagctcacAgtggacaagagcaggtggcagcaggggaaTgtcttctcatgcTCTg |
| | | tgatgcatgaggctctgcacaaccactacacCcagaagagcctctccctgtctccAgg |
| | | taaa |
| 5247 | DNA Constant | gcCtcTaccaagggcccatcTgtcttccccctggcaccctcctccaagagcacctctg |
| | Region of | ggggcacagcTgccctgggctgcctggtcaaggactacttcccTgaacctgtgacAgt |
| | Heavy Chain | ctcCtggaactcaggAgccctgaccagTggAgtgcacaccttcccTgctgtcctacag |
| | (hIgG1) | tcctcaggactctactccctcagcTCTgtggtgacAgtgccctccagcagcttgggca |
| | | cccagacctacatctgcaaTgtgaatcacaagcccagcaacaccaaggtggacaagaG |
| | | agttgagcccaaatcttgtgacaaaactcacacatgcccaccTtgcccagcacctgaa |
| | | ctGGtggggggaccAtcagtcttcctcCtccccccaaaacccaaggacaccctcatga |
| | | tctccAGAacccctgaggtcacatgTgtggtggtggaTgtgagccaTgaagaccctga |
| | | ggtcaagttcaactggtaTgtggaTggAgtggaggtgcataatgccaagacaaagccC |
| | | cCTgaggagcagtacaacagcacTCTcAGAgtggtcTCTgtcctcacAgtcctgcacc |
| | | aggactggctgaatggcaaggagtacaagtgcaaggtctccaacaaagccctcccagc |
| | | ccccatTgagaaaaccatctccaaagccaaagggcagcccAGAgaaccacaggtgtac |
| | | accctgcccccatccAGAgaTgagCtgaccaagaaccaggtcagcctgacctgcctgg |
| | | tcaaaggcttctatcccTCTgacatTgcTgtggagtgggagagcaatgggcagccTga |
| | | gaacaactacaagaccacAcctcTTgtgctggactcTgaTggctccttcttcctctac |
| | | agcaagctcacAgtggacaagagcagAtggcagcaggggaaTgtcttctcatgcTCTg |
| | | tgatgcatgaggctctgcacaaccactacacCcagaagagcctctccctgtctccAgg |
| | | taaa |
| 5220 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKRVEPKSCDKTHTCPPCPAPE |
| | Heavy Chain | LVGGPSVFLLPPKPKDTLMISRTPEVTCVVVDVSHEDPEVKFNWYVDGVEVHNAKTKP |
| | (hIgG1) | PEEQYNSTLRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPLVLDSDGSFFLY |
| | | SKLTVDKSRWQQGNVFSCSVMHEALHNHYTQKSLSLSPGK |
| 7591 | DNA Constant | gcCtcTaccaagggcccatcTgtcttccccctggcaccctcctccaagagcacctctg |
| | Region of | ggggcacagcTgccctgggctgcctggtcaaggactacttcccTgaacctgtgacAgt |
| | Heavy Chain | ctcCtggaactcaggAgccctgaccagTggAgtgcacaccttcccTgctgtcctacag |
| | (hIgG1) | tcctcaggactctactccctcagcTCTgtggtgacAgtgccctccagcagcttgggca |
| | | cccagacctacatctgcaaTgtgaatcacaagcccagcaacaccaaggtggacaagaG |
| | | agttgagcccaaatcttgtgacaaaactcacacatgcccaccTtgcccagcacctgaa |
| | | ctGGtggggggaccAtcagtcttcctcCtccccccaaaacccaaggacaccctcatgG |
| | | CAtccAGAacccctgaggtcacatgIgtggtggtggaTgtgagccaTgaagaccctga |
| | | ggtcaagttcaactggtaTgtggaTggAgtggaggtgcataatgccaagacaaagccC |
| | | cCTgaggagcagtacaacagcacTCTcAGAgtggtcTCTgtcctcacAgtcctgGCCc |
| | | aggactggctgaatggcaaggagtacaagtgcaaggtctccaacaaagccctcccagc |
| | | ccccatTgagaaaaccatctccaaagccaaagggcagcccAGAgaaccacaggtgtac |
| | | accctgcccccatccAGAgaTgagCtgaccaagaaccaggtcagcctgacctgcctgg |
| | | tcaaaggcttctatcccTCTgacatIgcTgtggagtgggagagcaatgggcagccTga |
| | | gaacaactacaagaccacAcctcTTgtgctggactcTgaTggctccttcttcctctac |
| | | agcaagctcacAgtggacaagagcagAtggcagcaggggaaTgtcttctcatgcTCTg |
| | | tgatgcatgaggctctgcacaacGCTtacacCcagaagagcctctccctgtctccAgg |
| | | taaa |
| 7592 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKRVEPKSCDKTHTCPPCPAPE |
| | Heavy Chain | LVGGPSVFLLPPKPKDTLMASRTPEVTCVVVDVSHEDPEVKFNWYVDGVEVHNAKTKP |
| | (hIgG1) | PEEQYNSTLRVVSVLTVLAQDWLNGKEYKCKVSNKALPAPIEKTISKAKGQPREPQVY |
| | | TLPPSRDELTKNQVSLTCLVKGFYPSDIAVEWESNGQPENNYKTTPLVLDSDGSFFLY |
| | | SKLTVDKSRWQQGNVFSCSVMHEALHNAYTQKSLSLSPGK |
| 5221 | DNA Constant | AGAactgtggctgcaccatctgtcttcatcttcccAccatctgatgagcagttgaaat |
| | Region of Light | ctggaactgcctctgttgtgtgcctgctgaataacttctatcccagagaggccaaagt |
| | Chain | acagtggaaggtggataaTgccctccaatcAggtaactcccaggagagtgtcacagag |
| | | caggacagcaaggacagcacctacagcctcagcagcaccctgacTctgagcaaagcag |
| | | actaTgagaaacacaaagtctaTgcctgTgaagtcacccatcagggcctgagctcTcc |
| | | Tgtcacaaagagcttcaacaggggagagtgt |
| 5222 | Constant | RTVAAPSVFIFPPSDEQLKSGTASVVCLLNNFYPREAKVQWKVDNALQSGNSQESVTE |
| | Region of Light | QDSKDSTYSLSSTLTLSKADYEKHKVYACEVTHQGLSSPVTKSENRGEC |
| | Chain | |
| 5277 | DNA Constant | gagcccaaatcttgtgacaaaactTACacatgcccaccgtgcccagcacctgaactcG |
| | Region of | tggggggaccgtcagtcttcctcCtccccccaaaacccaaggacaccctcatgatctc |
| | Heavy Chain | ccggacccctgaggtcacatgcgtggtggtggacgtgagccacgaagaccctgaggtc |
| | (hIgG1) | aagttcaactggtacgtggacggcgtggaggtgcataatgccaagacaaagccgcCgg |
| | | aggagcagtacaacagcacgCTccgtgtggtcagcgtcctcaccgtcctgcaccagga |
| | | ctggctgaatggcaaggagtacaagtgcaaggtctccaacaaagccctcccagccccc |
| | | atcgagaaaaccatctccaaagccaaagggcagccccgagaaccacaggtgtacaccc |
| | | tgcccccatcccgggaTgagCtgaccaagaaccaggtcagcctgacctgcctggtcaa |
| | | aggcttctatcccagcgacatcgccgtggagtgggagagcaatgggcagccggagaac |
| | | aactacaagaccacgcctcTcgtgctggactccgacggctccttcttcctctacagca |
| | | agctcaccgtggacaagagcaggtggcagcaggggaacgtcttctcatgctccgtgat |
| | | gcatgaggctctgcacaaccactacacgcagaagagcctctccctgtctccgggtaaa |
| 5278 | Constant | EPKSCDKTYTCPPCPAPELVGGPSVFLLPPKPKDTLMISRTPEVTCVVVDVSHEDPEV |
| | Region of | KFNWYVDGVEVHNAKTKPPEEQYNSTLRVVSVLTVLHQDWLNGKEYKCKVSNKALPAP |
| | Heavy Chain | IEKTISKAKGQPREPQVYTLPPSRDELTKNQVSLTCLVKGFYPSDIAVEWESNGQPEN |
| | | NYKTTPLVLDSDGSFFLYSKLTVDKSRWQQGNVFSCSVMHEALHNHYTQKSLSLSPGK |
| 5279 | DNA Constant | gagcccaaatcttgtgacaaaactTACacatgcccaccgtgcccagcacctgaactcG |
| | Region of | tggggggaccgtcagtcttcctcCtccccccaaaacccaaggacaccctcatgatctc |
| | Heavy Chain | ccggacccctgaggtcacatgcgtggtggtggacgtgagccacgaagaccctgaggtc |
| | (hIgG1) | aagttcaactggtacgtggacggcgtggaggtgcataatgccaagacaaagccgcCgg |
| | | aggagcagtacaacagcacgCTccgtgtggtcagcgtcctcaccgtcctgcaccagga |
| | | ctggctgaatggcaaggagtacaagtgcaaggtctccaacaaagccctcccagccccc |
| | | atcgagaaaaccatctccaaagccaaagggcagccccgagaaccacaggtgtacaccc |
| | | tgcccccatcccgggaTgagCtgaccaagaaccaggtcagcctgacctgcctggtcaa |
| | | aggcttctatcccagcgacatcgccgtggagtgggagagcaatgggcagccggagaac |
| | | aactacaagaccacgcctcTcgtgctggactccgacggctccttcttcctctacagca |
| | | agctcaccgtggacaagagcaggtggcagcaggggaacgtcttctcatgctccgtgat |
| | | gcatgaggctctgcacaaccactacacgcagaagagcctctccctgtctccgggtaaa |
| 5280 | Constant | EPKSCDKTYTCPPCPAPELVGGPSVFLLPPKPKDTLMISRTPEVTCVVVDVSHEDPEV |
| | Region of | KFNWYVDGVEVHNAKTKPPEEQYNSTLRVVSVLTVLHQDWLNGKEYKCKVSNKALPAP |
| | Heavy Chain | IEKTISKAKGQPREPQVYTLPPSRDELTKNQVSLTCLVKGFYPSDIAVEWESNGQPEN |
| | | NYKTTPLVLDSDGSFFLYSKLTVDKSRWQQGNVFSCSVMHEALHNHYTQKSLSLSPGK |
| 5344 | DNA Constant | GCGTCGACCAAGGGGCCCTCGGTCTTCCCCCTGGCACCCTCCTCCAAGAGCACCTCTG |
| | Region of | GGGGCACAGCGGCCCTGGGCTGCCTGGTCAAGGACTACTTCCCCGAACCGGTGACGGT |
| | Heavy Chain | GTCGTGGAACTCAGGCGCCCTGACCAGCGGCGTGCACACCTTCCCGGCTGTCCTACAG |
| | | TCCTCAGGACTCTACTCCCTCAGCAGCGTGGTGACCGTGCCCTCCAGCAGCTTGGGCA |
| | | CCCAGACCTACATCTGCAACGTGAATCACAAGCCCAGCAACACCAAGGTGGACAAGAA |
| | | AGTTGAGCCCAAATCTTGTGACAAAACTCACACATGCCCACCGTGCCCAGCACCTGAA |
| | | CTCCTGGGGGGACCGTCAGTCTTCCTCTTCCCCCCAAAACCCAAGGACACCCTCATGA |
| | | TCTCCCGGACCCCTGAGGTCACATGCGTGGTGGTGGACGTGAGCCACGAAGACCCTGA |
| | | GGTCAAGTTCAACTGGTACGTGGACGGCGTGGAGGTGCATAATGCCAAGACAAAGCCG |
| | | CGGGAGGAGCAGTACAACAGCACGTACCGTGTGGTCAGCGTCCTCACCGTCCTGCACC |
| | | AGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTAC |
| | | ACCCTGCCCCCATCCCGGGATGAGCTGACCAAGAACCAGGTCAGCCTGACCTGCCTGG |
| | | TCAAAGGCTTCTATCCCAGCGACATCGCCGTGGAGTGGGAGAGCAATGGGCAGCCGGA |
| | | GAACAACTACAAGACCACGCCTCCCGTGCTGGACTCCGACGGCTCCTTCTTCCTCTAC |
| | | AGCAAGCTCACCGTGGACAAGAGCAGGTGGCAGCAGGGGAACGTCTTCTCATGCTCCG |
| | | TGATGCATGAGGCTCTGCACAACCACTACACGCAGAAGAGCCTCTCCCTGTCTCCCGG |
| | | CAAG |
| 5345 | Constant | ASTKGPSVFPLAPSSKSTSGGTAALGCLVKDYFPEPVTVSWNSGALTSGVHTFPAVLQ |
| | Region of | SSGLYSLSSVVTVPSSSLGTQTYICNVNHKPSNTKVDKKVEPKSCDKTHTCPPCPAPE |
| | Heavy Chain | LLGGPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 5346 | DNA Constant | CGTACGGTGGCTGCACCATCTGTCTTCATCTTCCCGCCATCTGATGAGCAGTTGAAAT |
| | Region of Light | CTGGAACTGCCTCTGTTGTGTGCCTGCTGAATAACTTCTACCCCAGAGAAGCCAAAGT |
| | Chain | GCAGTGGAAGGTGGACAACGCCCTGCAGAGCGGAAACAGCCAGGAAAGCGTGACAGAG |
| | | CAGGATTCCAAGGATTCCACATACAGCCTGAGCAGCACACTGACACTGTCCAAGGCCG |
| | | ACTACGAGAAGCACAAGGTGTACGCCTGCGAAGTGACACACCAGGGACTGTCCTCCCC |
| | | TGTGACAAAGAGCTTCAACAGAGGAGAATGC |

### Payload Component: SH3-Derived Binding Polypeptide

In some embodiments, the nucleic acid encoding the payload, e.g., antibody molecule, comprises a nucleotide sequence encoding a Fyn SH3-derived binding polypeptide (e.g., a Fynomer sequence or Fynomer sequence region herein). Fynomers are small T-kDa globular proteins derived from the SH3 domain of the human Fyn kinase (Fyn SH3) that can be engineered to bind with antibody-like affinity and specificity to a target of choice through mutation of two loops (RT- and src-loop) on the surface of the Fyn SH3 domain (Brack et al. (2014), Mol Cancer Ther 13(8):2030-2039). Fynomers may be used to generate multispecific FynomAbs, which are obtained by fusion of Fynomers to any of the four antibody light- or heavy-chain termini. Id.

In some embodiments, the nucleic acid encoding the payload, e.g., antibody molecule, comprises a C12 Fynomer region. The HER2-specific Fynomer C12 is a 63-amino acid polypeptide that targets an epitope in domain I of HER2. In some embodiments, the Fynomer sequence encodes the fusion of Fynomer C12 to an antibody C- or N-termini heavy or light chain. In one embodiment the nucleotide sequence encodes the fusion of Fynomer C12 to the N-terminus of the light chain of an antibody. In another embodiment, the nucleotide sequence encodes the fusion of C12 to the N-terminus of the light chain of an antibody. The anti-proliferative activity of anti-HER2 Fynomer-antibody fusions varies depending on the relative orientation of the Fynomer and the binding site of the antibody.

In some embodiments, the encoded C12 Fynomer comprises the amino acid sequence of SEQ ID NO: 5156, or an amino acid sequence with at least 70%, 75%, 80%, 85%, 90%, 95%, or 99% sequence identity thereto. In some embodiments, the nucleotide sequence encoding the C12 Fynomer comprises the nucleotide sequence of SEQ ID NO: 5155, or a nucleotide sequence with at least 70%, 75%, 80%, 85%, 90%, 95%, or 99% sequence identity thereto. In some embodiments, the Fynomer region comprises about 100-200 nucleotides in length, e.g., about 110-200 nucleotides, about 120-200 nucleotides, about 130-200 nucleotides, about 140-200 nucleotides, about 150-200 nucleotides, about 160-200 nucleotides, about 170-200 nucleotides, about 170-200 nucleotides, about 180-200 nucleotides, about 185-200 nucleotides, about 180-190 nucleotides, or about 185-190 nucleotides. In some embodiments, the Fynomer region comprises about 180 nucleotides to about 200 nucleotides in length, e.g., about 189 nucleotides.

### Payload Component: Linkers

In some embodiments, the nucleic acid encoding the payload, e.g., antibody molecule, comprises a nucleotide sequence encoding a linker. In some embodiments, the nucleic acid encoding the payload encodes two or more linkers. In some embodiments, the encoded linker comprises a linker provided in Table 41. In some embodiments, the encoded linker comprises an amino acid sequence encoded by any one of the nucleotide sequences provided in Table 41, or an amino acid sequence with at least 70%, 75%, 80%, 85%, 90%, 95%, or 99% sequence identity thereto. In some embodiments, the nucleotide sequence encoding the linker comprises any one of the nucleotide sequences provided in Table 41, or a nucleotide sequence with at least 70%, 75%, 80%, 85%, 90%, 95%, or 99% sequence identity thereto.

TABLE 41
Linkers

| Linker | | | SEQ | |
|---|---|---|---|---|
| ID | Description | Length | ID NO | Sequence |
| Linker1 | Furin | 12 | 1724 | AGAAAGAGGCGA |
| Linker2 | Furin | 12 | 1725 | CGGGCCAAGCGG |
| | Furin | 12 | 6557 | AGAAAGAGGAGA |
| Linker3 | T2A | 54 | 1726 | GAGGGCAGAGGAAGTCTTCTAACATGCGGTGACGTGGAGG |
| | | | | AGAATCCCGGCCCT |
| | T2A | 54 | 6558 | GAGGGCAGAGGAAGTCTTCTAACATGTGGTGATGTGGAGG |
| | | | | AGAATCCTGGCCCT |
| Linker4 | F2A | 75 | 1727 | GGAAGCGGAGTGAAACAGACTTTGAATTTTGACCTTCTCA |
| | | | | AGTTGGCGGGAGACGTGGAGTCCAACCCTGGACCT |
| Linker5 | P2A | 66 | 1728 | GGAAGCGGAGCTACTAACTTCAGCCTGCTGAAGCAGGCTG |
| | | | | GAGACGTGGAGGAGAACCCTGGACCT |
| Linker6 | SG4S (SEQ | 18 | 1729 | TCCGGAGGCGGCGGCAGC |
| | ID NO: 4536) | | | |
| Linker7 | (G4S)3 (SEQ | 45 | 1730 | GGTGGTGGTGGTAGCGGCGGCGGCGGCTCTGGTGGTGGTG |
| | ID NO: 4537) | | | GATCC |
| Linker8 | (G4S)5 (SEQ | 75 | 1731 | GGCGGAGGTGGCTCCGGAGGCGGAGGCAGCGGCGGAGGTG |
| | ID NO: 4538) | | | GGTCTGGCGGAGGCGGGTGCGGCGGAGGTGGCTCC |
| Linker9 | IRES | 609 | 1732 | AATTCCGCCCCTCTCCCCCCCCCCCCTCTCCCTCCCCCCC |
| | | | | CCCTAACGTTACTGGCCGAAGCCGCTTGGAATAAGGCCGG |
| | | | | TGTGCGTTTGTCTATATGTTATTTTCCACCATATTGCCGT |
| | | | | CTTTTGGCAATGTGAGGGCCCGGAAACCTGGCCCTGTCTT |
| | | | | CTTGACGAGCATTCCTAGGGGTCTTTCCCCTCTCGCCAAA |
| | | | | GGAATGCAAGGTCTGTTGAATGTCGTGAAGGAAGCAGTTC |
| | | | | CTCTGGAAGCTTCTTGAAGACAAACAACGTCTGTAGCGAC |
| | | | | CCTTTGCAGGCAGCGGAACCCCCCACCTGGCGACAGGTGC |
| | | | | CTCTGCGGCCAAAAGCCACGTGTATAAGATACACCTGCAA |
| | | | | AGGCGGCACAACCCCAGTGCCACGTTGTGAGTTGGATAGT |
| | | | | TGTGGAAAGAGTCAAATGGCTCTCCTCAAGCGTATTCAAC |
| | | | | AAGGGGCTGAAGGATGCCCAGAAGGTACCCCATTGTATGG |
| | | | | GATCTGATCTGGGGCCTCGGTGCACATGCTTTACATGTGT |
| | | | | TTAGTCGAGGTTAAAAAAACGTCTAGGCCCCCCGAACCAC |
| | | | | GGGGACGTGGTTTTCCTTTGAAAAACACGATGATAATATG |
| | | | | GCCACAACC |
| Linker10 | IRES-2 | 623 | 1733 | TAACGAATTCCGCCCCTCTCCCCCCCCCCCCTCTCCCTCC |
| | | | | CCCCCCCCTAACGTTACTGGCCGAAGCCGCTTGGAATAAG |
| | | | | GCCGGTGTGCGTTTGTCTATATGTTATTTTCCACCATATT |
| | | | | GCCGTCTTTTGGCAATGTGAGGGCCCGGAAACCTGGCCCT |
| | | | | GTCTTCTTGACGAGCATTCCTAGGGGTCTTTCCCCTCTCG |
| | | | | CCAAAGGAATGCAAGGTCTGTTGAATGTCGTGAAGGAAGC |
| | | | | AGTTCCTCTGGAAGCTTCTTGAAGACAAACAACGTCTGTA |
| | | | | GCGACCCTTTGCAGGCAGCGGAACCCCCCACCTGGCGACA |
| | | | | GGTGCCTCTGCGGCCAAAAGCCACGTGTATAAGATACACC |
| | | | | TGCAAAGGCGGCACAACCCCAGTGCCACGTTGTGAGTTGG |
| | | | | ATAGTTGTGGAAAGAGTCAAATGGCTCTCCTCAAGCGTAT |
| | | | | TCAACAAGGGGCTGAAGGATGCCCAGAAGGTACCCCATTG |
| | | | | TATGGGATCTGATCTGGGGCCTCGGTGCACATGCTTTACA |
| | | | | TGTGTTTAGTCGAGGTTAAAAAAACGTCTAGGCCCCCCGA |
| | | | | ACCACGGGGACGTGGTTTTCCTTTGAAAAACACGATGATA |
| | | | | ATATGGCCACAACCGCCGCCACC |
| Linker11 | hIgG2 hinge | 54 | 1734 | GTGGAGAGGAAGTGCTGCGTGGAGTGCCCACCATGCCCTG |
| | | | | CCCCTCCTGTGGCC |
| Linker12 | hIgG3 hinge | 108 | 1735 | GAGCTCAAAACCCCACTTGGTGACACAACTCACACATGCC |
| | | | | CACGGTGCCCAGAGCCCAAATCTTGTGACACACCTCCCCC |
| | | | | GTGCCCACGGTGCCCAGCACCTGAACTC |
| Linker13 | hIgG3-2 | 153 | 1736 | GAGCTCAAAACCCCACTTGGTGACACAACTCACACATGCC |
| | hinge | | | CACGGTGCCCAGAGCCCAAATCTTGTGACACACCTCCCCC |
| | | | | GTGCCCACGGTGCCCAGAGCCCAAATCTTGTGACACACCT |
| | | | | CCCCCATGCCCACGGTGCCCAGCACCTGAACTC |
| Linker14 | hIgG3-3 | 198 | 1737 | GAGCTCAAAACCCCACTTGGTGACACAACTCACACATGCC |
| | hinge | | | CACGGTGCCCAGAGCCCAAATCTTGTGACACACCTCCCCC |
| | | | | GTGCCCACGGTGCCCAGAGCCCAAATCTTGTGACACACCT |
| | | | | CCCCCATGCCCACGGTGCCCAGAGCCCAAATCTTGTGACA |
| | | | | CACCTCCCCCGTGCCCAAGGTGCCCAGCACCTGAACTC |
| Linker15 | msiGG-1 | 45 | 1738 | GTGCCCAGGGATTGTGGTTGTAAGCCTTGCATATGTACAG |
| | hinge | | | TCCCA |
| Linker16 | msiGG1 | 18 | 1739 | GTGCCCAGGGATTGTGGT |
| | hingc | | | |
| Linker17 | HigG3 hinge | 198 | 6596 | GAGCTCAAAACCCCACTTGGTGACACAACTCACACATGCC |
| | | | | CACGGTGCCCAGAGCCCAAATCTTGTGACACACCTCCCCC |
| | | | | GTGCCCACGGTGCCCAGAGCCCAAATCTTGTGACACAACT |
| | | | | CACACATGCCCACGGTGCCCAGAGCCCAAATCTTGTGACA |
| | | | | CACCTCCCCCGTGCCCAAGGTGCCCAGCACCTGAACTC |
| Linker18 | G4S (SEQ ID | 15 | 6597 | GGTGGTGGTGGATCC |
| | NO: 4535) | | | |
| Linker19 | (G4S)2 (SEQ | 30 | 6598 | GGTGGTGGTGGATCCGGTGGTGGTGGATCC |
| | ID NO: 4539) | | | |
| Linker20 | (G4S)3 (SEQ | 45 | 6599 | GGTGGTGGTGGATCCGGTGGTGGTGGATCCGGTGGTGGTG |
| | ID NO: 4537) | | | GATCC |
| Linker21 | (G4S)4 (SEQ | 60 | 6600 | GGTGGTGGTGGATCCGGTGGTGGTGGATCCGGTGGTGGTG |
| | ID NO: 4540) | | | GATCCGGTGGTGGTGGATCC |
| Linker22 | (G4S)5 (SEQ | 75 | 6601 | GGTGGTGGTGGTAGCGGCGGCGGCGGCTCTGGTGGTGGTG |
| | ID NO: 4538) | | | GATCCGGTGGTGGTGGATCCGGTGGTGGTGGATCC |
| Linker23 | (G4S)5 (SEQ | 75 | 6602 | GGTGGTGGTGGATCCGGTGGTGGTGGATCCGGTGGTGGTG |
| | ID NO: 4538) | | | GATCCGGTGGTGGTGGATCCGGTGGTGGTGGATCC |
| Linker24 | (G4S)6 (SEQ | 90 | 6603 | GGTGGTGGTGGTAGCGGCGGCGGCGGCTCTGGTGGTGGTG |
| | ID NO: 4541) | | | GATCCGGTGGTGGTGGATCCGGTGGTGGTGGATCCGGTGG |
| | | | | TGGTGGATCC |
| Linker25 | (G4S)8 (SEQ | 120 | 6604 | GGTGGTGGTGGTAGCGGCGGCGGCGGCTCTGGTGGTGGTG |
| | ID NO: 4542) | | | GATCCGGTGGTGGTGGATCCGGTGGTGGTGGATCCGGTGG |
| | | | | TGGTGGATCCGGTGGTGGTGGATCCGGTGGTGGTGGATCC |
| Linker26 | (G4S)8 (SEQ | 120 | 6605 | GGTGGTGGTGGATCCGGTGGTGGTGGATCCGGTGGTGGTG |
| | ID NO: 4542) | | | GATCCGGTGGTGGTGGATCCGGTGGTGGTGGATCCGGTGG |
| | | | | TGGTGGATCCGGTGGTGGTGGATCCGGTGGTGGTGGATCC |
| Linker27 | (G4S)4 (SEQ | 60 | 6606 | GGTGGTGGTGGTAGCGGCGGCGGCGGCTCTGGTGGTGGTG |
| | ID NO: 4540) | | | GATCCGGTGGTGGTGGATCC |
| Linker28 | (G4S)6 (SEQ | 90 | 6610 | GGTGGTGGTGGATCCGGTGGTGGTGGATCCGGTGGTGGTG |
| | ID NO: 4541) | | | GATCCGGTGGTGGTGGATCCGGTGGTGGTGGATCCGGTGG |
| | | | | TGGTGGATCC |
| Linker | G4S (SEQ ID | 15 | 5161 | GGAGGAGGAGGAAGT |
| 29 | NO: 4535) | | | |
| Linker | G4S (SEQ ID | 15 | 5162 | GGAGGTGGAGGTTCT |
| 30 | NO: 4535) | | | |
| Linker | GS | 6 | | GGCTCT |
| 31 | | | | |
| Linker | (G4S)3 (SEQ | 45 | 5347 | GGTGGTGGTGGATCCGGAGGAGGAGGAAGTGGAGGTGGAG |
| 32 | ID NO: 4537) | | | GTTCT |
| (DNA) | | | | |
| Linker | (G4S)3 (SEQ | 15 | 5348 | GGGGSGGGGSGGGGS |
| 33 | ID NO: 4537) | | | |
| (amino | | | | |
| acid) | | | | |

In some embodiments, exemplary linker sequences of any of the construes described herein include Linker 29 (SEQ ID NO: 5161); Linker 30 (SEQ ID NO: 5162); Linker 31 (GGCTCT); Linker 32 (DNA; SEQ ID NO: 5347); Linker 32 (amino acid; SEQ ID NO: 5348).

In some embodiments, any of the antibody molecules described herein can have a linker. e.g., a flexible polypeptide linker, of varying lengths, connecting the variable domains (e.g., the VH and the VL) of the antigen binding domain of the antibody molecule. For example, a (Gly4-Ser)n linker, wherein n is 0, 1, 2, 3, 4, 5, 6, 7, or 8 (SEQ ID NO: 7587) can be used (e.g., any one of SEQ ID NOs: 1730-1731, 6597-6606, 6610, 5161-5162, 5347, or 5348). In some embodiments, the antibody molecule binds to HER2.

In some embodiments, the encoded linker comprises an enzymatic cleavage site, e.g., for intracellular and/or extracellular cleavage. In some embodiments, the linker is cleaved to separate the VH and the VL of the antigen binding domain and/or the heavy chain and light chain of the antibody molecule (e.g., an anti-HER2 antibody molecule). In some embodiments, the encoded linker comprises a furin linker or a functional variant. In some embodiments, the nucleotide sequence encoding the furin linker comprises the nucleotide sequence of SEQ ID NO: 1724, or a nucleotide sequence with at least 70%, 75%, 80%, 85%, 90%, 95%, or 99% sequence identity thereto. In some embodiments, furin cleaves proteins downstream of a basic amino acid target sequence (e.g., Arg-X-(Arg/Lys)-Arg) (e.g., as described in Thomas, G., 2002. Nature Reviews Molecular Cell Biology 3(10): 753-66; the contents of which are herein incorporated by reference in its entirety). In some embodiments, the encoded linker comprises a 2A self-cleaving peptide (e.g., a 2A peptide derived from foot-and-mouth disease virus (F2A), porcine teschovirus-1 (P2A), Thoseaasigna virus (T2A), or equine rhinitis A virus (E2A)). In some embodiments, the encoded linker comprises a T2A self-cleaving peptide linker. In some embodiments, the nucleotide sequence encoding the T2A linker comprises the nucleotide sequence of SEQ ID NO: 1726, or a nucleotide sequence with at least 70%, 75%, 80%, 85%, 90%, 95%, or 99% sequence identity thereto. In some embodiments, the nucleic acid encoding the payload encodes a furin linker and a T2A linker.

In some embodiments, the encoded linkers comprises a cathepsin, a matrix metalloproteinases or a legumain cleavage sites, such as those described e.g. by Cizeau and Macdonald in International Publication No. WO2008052322, the contents of which are herein incorporated in their entirety.

In some embodiments, the encoded linker comprises an internal ribosomal entry site (IRES) is a nucleotide sequence (>500 nucleotides) for initiation of translation in the middle of a nucleotide sequence, e.g., an mRNA sequence (Kim, J. H. et al., 2011. PLoS One 6(4): e18556; the contents of which are herein incorporated by reference in its entirety), which can be used, for example, to modulate expression of one or more transgenes. In some embodiments, the encode linker comprises a small and unbranched serine-rich peptide linker, such as those described by Huston et al. in U.S. Pat. No. 5,525,491, the contents of which are herein incorporated in their entirety. In some embodiments, polypeptides comprising a serine-rich linker has increased solubility. In some embodiments, the encoded linker comprises an artificial linker, such as those described by Whitlow and Filpula in U.S. Pat. No. 5,856,456 and Ladner et al. in U.S. Pat. No. 4,946,778, the contents of each of which are herein incorporated by their entirety.

### Payload Component: Signal Sequences

In some embodiments, the nucleotide sequence comprising the transgene encoding the payload, e.g., an antibody molecule, comprises a nucleotide sequence encoding a signal sequence (e.g., a signal sequence region herein). In some embodiments, the nucleotide sequence comprising the transgene encoding the payload comprises two signal sequence regions. In some embodiments, the nucleotide sequence comprising the transgene encoding the payload comprises three or more signal sequence regions.

In some embodiments, the nucleotide sequence encoding the signal sequence is located 5′ relative to the nucleotide sequence encoding the VH and/or the heavy chain. In some embodiments, the nucleotide sequence encoding the signal sequence is located 5′ relative to the nucleotide sequence encoding the VL and/or the light chain. In some embodiments, the encoded VH, VL, heavy chain, and/or light chain of the encoded antibody molecule comprises a signal sequence at the N-terminus, wherein the signal sequence is optionally cleaved during cellular processing and/or localization of the antibody molecule.

In some embodiments, the signal sequence comprises any one of the signal sequences provided in Table 11A or a functional variant thereof. In some embodiments, the encoded signal sequence comprises an amino acid sequence encoded by any one of the nucleotide sequences provided in Table 11A, or an amino acid sequence with at least 70%, 75%, 80%, 85%, 90%, 95%, or 99% sequence identity thereto. In some embodiments, the nucleotide sequence encoding the signal sequence comprises any one of the nucleotide sequences provided in Table 11A, or a nucleotide sequence with at least 70%, 75%, 80%, 85%, 90%, 95%, or 99% sequence identity thereto.

TABLE 11A
Signal Sequence Regions

| | | SEQ | |
|---|---|---|---|
| | | ID | |
| Name | Length | NO | Sequence |
| Signal 17 | 57 NTs | 5157 | ATGGACTGGACCTGGAGG |
| | | | TTCCTCTTTGTGGTGGCA |
| | | | GCAGCTACAGGTGTCCAG |
| | | | TCC |
| Signal 17 | 19 | 5158 | MDWTWRFLFVVAAATGVQ |
| (amino | amino | | S |
| acid) | acids | | |
| Signal 17a | 57 NTs | 5032 | ATGGACTGGACCTGGAGG |
| | | | TTCCTCTTTGTGGTGGCA |
| | | | GCAGCTACAGGTGTCCAG |
| | | | TCT |
| Signal 18 | 66 NTs | 5159 | ATGGACATGAGGGTCCCT |
| | | | GCTCAGCTCCTGGGGCTC |
| | | | CTGCTGCTCTGGCTCTCA |
| | | | GGTGCCAGATGT |
| Signal 18 | 22 amino | 5160 | MDMRVPAQLLGLLLLWLS |
| (amino | amino | | GARC |
| acid) | acids | | |

In some embodiments, the nucleotide sequence encoding the signal sequence comprises the nucleotide sequence of SEQ ID NO: 5157, or nucleotide sequence with at least 70%, 75%, 80%, 85%, 90%, 95%, or 99% sequence identity thereto. In some embodiments, the nucleotide sequence encoding the signal sequence comprises the nucleotide sequence of SEQ ID NO: 5032, or nucleotide sequence with at least 70%, 75%, 80%, 85%, 90%, 95%, or 99% sequence identity thereto. In some embodiments, the nucleotide sequence encoding the signal sequence comprises the nucleotide sequence of SEQ ID NO: 5159, or nucleotide sequence with at least 70%, 75%, 80%, 85%, 90%, 95%, or 99% sequence identity thereto. In some embodiments, the nucleotide sequence encoding the signal sequence comprises the nucleotide sequence of SEQ ID NO: 5157 and is located 5′ relative to the nucleotide sequence encoding the VH and/or the heavy chain of the antibody molecule. In some embodiments, the nucleotide sequence encoding the signal sequence comprises the nucleotide sequence of SEQ ID NO: 5159 and is located 5′ relative to the nucleotide sequence encoding the VL and/or the light chain of the antibody molecule.

In some embodiments, the encoded signal sequence comprises the amino acid sequence of SEQ ID NO: 5158, or amino acid sequence with at least 70%, 75%, 80%, 85%, 90%, 95%, or 99% sequence identity thereto. In some embodiments, the encoded signal sequence comprises the amino acid sequence of SEQ ID NO: 5160, or amino acid sequence with at least 70%, 75%, 80%, 85%, 90%, 95%, or 99% sequence identity thereto. In some embodiments, the encoded sequence comprises the amino acid sequence of SEQ ID NO: 5158 and is located at the N-terminus of the amino acid sequence of the encoded VH and/or heavy chain of the antibody molecule. In some embodiments, the encoded sequence comprises the amino acid sequence of SEQ ID NO: 5160 and is located at the N-terminus of the amino acid sequence of the encoded VL and/or light chain of the antibody molecule.

### AAV Particles Comprising a Genetic Element Encoding an Anti-HER2 Antibody Molecule

In some embodiments, the AAV particle comprises a genetic element comprising a nucleotide sequence comprising a transgene encoding a payload comprising an anti-HER2 antibody molecule. In some embodiments, the genetic element is replicated and packaged into an AAV particle. A cell, e.g., a target cell, transduced with an AAV particle comprising an anti-HER2 antibody molecule may express the encoded antibody molecule in the cell or secrete the encoded antibody molecule.

In some embodiments, the genetic element of an AAV particle described herein comprises a nucleotide sequence, e.g., a nucleotide sequence from the 5′ ITR to the 3′ ITR, comprising any of the nucleotide sequences of HER-04, HER-05, HER-10, HER-15, HER-42, HER-43, HER-46, HER-47, HER-53, HER-57, HER-62, HER-69, HER-73, HER-75, HER-77, HER-78, HER-82, HER-88, HER-89, HER-90, HER-91, HER-92, HER-97, HER-98, HER-99, HER-100, or HER-101, e.g., as described in Table 6, or a sequence substantially identical (e.g., having at least about 70%, 75%, 80%, 85%, 90%, 92%, 95%, 97%, 98%, or 99% sequence identity) to any of the aforesaid sequences. In some embodiments, the genetic element of an AAV particle described herein comprises a nucleotide sequence, e.g., a nucleotide sequence from the 5′ ITR to the 3′ ITR, comprising any of the nucleotide sequences in Table 6, or a sequence substantially identical (e.g., having at least about 70%, 75%, 80%, 85%, 90%, 92%, 95%, 97%, 98%, or 99% sequence identity) to any of the aforesaid sequences. In some embodiments, the genetic element of an AAV particle described herein comprises a nucleotide sequence, e.g., a nucleotide sequence from the 5′ ITR to the 3′ ITR, comprising any of the nucleotide sequences of SEQ ID NO: 5163, 5170, 5164, 5165, 5166, 5185, 5186, 5167, 5168, 5187, 5188, 5619, 5189, 5190, 5343, 5374, 5375, 6500, 6501, 6502, 6503, 6504, 6505, 6506, 6507, 6508, or 6509, or a sequence substantially identical (e.g., having at least about 70%, 75%, 80%, 85%, 90%, 92%, 95%, 97%, 98%, or 99% sequence identity) to any of the aforesaid sequences.

The present disclosure also provides in some embodiments, an antibody molecule that binds to HER2 encoded by any one of SEQ ID NOs: 5163, 5170, 5164, 5165, 5166, 5185, 5186, 5167, 5168, 5187, 5188, 5619, 5189, 5190, 5343, 5374, 5375, 6500, 6501, 6502, 6503, 6504, 6505, 6506, 6507, 6508, or 6509, or a sequence substantially identical (e.g., having at least about 70%, 75%, 80%, 85%, 90%, 92%, 95%, 97%, 98%, or 99% sequence identity) to any of the aforesaid sequences.

In some embodiments, the genetic element, e.g., any of the genetic elements described in Tables 6, 17-19, 23-26, 40, 42, 43, 68, and 69 may further comprise a Kozak sequence, a stuffer sequence, and/or a filler sequence.

In some embodiments, the genetic element comprises, in the 5′ to 3′ order, a 5′ ITR, a promoter region, an optional intronic region and/or exon region, a signal sequence, an antibody heavy chain region, a linker region, a signal sequence, an antibody light chain region, a polyadenylation sequence, an optional filler sequence, and a 3′ ITR. In some embodiments, the genetic element comprises, in the 5′ to 3′ order, a 5′ ITR, a promoter region, an optional intronic region and/or exon region, a signal sequence, an antibody light chain region, a linker region, a signal sequence, an antibody heavy chain region, a polyadenylation sequence, an optional filler sequence, and a 3′ ITR.

TABLE 6
ITR to ITR Sequences of AAV particles comprising anti-HER2 antibody polynucleotide
sequences

| ITR to | | ITR to | |
|---|---|---|---|
| ITR | | ITR | |
| Construct | Length | SEQ | |
| Name | (nt) | ID NO | Sequence |
| HER-04 | 3992 | 5163 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaaagttgagcccCCCaaatcttgtgacaaaact |
| | | | cacacatgcccaccgtgcccagcacctgaactcctggggggaccgtcagtcttcctct |
| | | | tccccccaaaacccaaggacaccctcatgatctcccggacccctgaggtcacatgcgt |
| | | | ggtggtggacgtgagccacgaagaccctgaggtcaagttcaactggtacgtggacggc |
| | | | gtggaggtgcataatgccaagacaaagccgcgggaggagcagtacaacagcacgtacc |
| | | | gtgtggtcagcgtcctcaccgtcctgcaccaggactggctgaatggcaaggagtacaa |
| | | | gtgcaaggtctccaacaaagccctcccagcccccatcgagaaaaccatctccaaagcc |
| | | | aaagggcagccccgagaaccacaggtgtacaccctgcccccatcccgggatgagctga |
| | | | ccaagaaccaggtcagcctgacctgcctggtcaaaggcttctatcccagcgacatcgc |
| | | | cgtggagtgggagagcaatgggcagccggagaacaactacaagaccacgcctcccgtg |
| | | | ctggactccgacggctccttcttcctctacagcaagctcaccgtggacaagagcaggt |
| | | | ggcagcaggggaacgtcttctcatgctccgtgatgcatgaggctctgcacaaccacta |
| | | | cacgcagaagagcctctccctgtctccgggtaaaagaaagaggcgagagggcagagga |
| | | | agtcttctaacatgcggtgacgtggaggagaatcccggccctATGGACATGAGGGTCC |
| | | | CTGCTCAGCTCCTGGGGCTCCTGCTGCTCTGGCTCTCAGGTGCCAGATGTgatatcca |
| | | | gatgacccagtccccgagctccctgtccgcctctgtgggcgatagggtcaccatcacc |
| | | | tgccgtgccagtcaggatgtgaatactgctgtagcctggtatcaacagaaaccaggaa |
| | | | aagctccgaaactactgatttactcggcatccttcctcTACtctggagtcccttctcg |
| | | | cttctctggatccagatctgggacggatttcactctgaccatcagcagtctgcagccg |
| | | | gaagacttcgcaacttattactgtcagcaacattatactactcctcccacgttcggac |
| | | | agggtaccaaggtggagatcaaacgaactgtggctgcaccatctgtcttcatcttccc |
| | | | gccatctgatgagcagttgaaatctggaactgcctctgttgtgtgcctgctgaataac |
| | | | ttctatcccagagaggccaaagtacagtggaaggtggataacgccctccaatcgggta |
| | | | actcccaggagagtgtcacagagcaggacagcaaggacagcacctacagcctcagcag |
| | | | caccctgacgctgagcaaagcagactacgagaaacacaaagtctacgcctgcgaagtc |
| | | | acccatcagggcctgagctcgcccgtcacaaagagcttcaacaggggagagtgttagt |
| | | | agtaactcgaggacggggtgaactacgcctgaggatccgatctttttccctctgccaa |
| | | | aaattatggggacatcatgaagccccttgagcatctgacttctggctaataaaggaaa |
| | | | tttattttcattgcaatagtgtgttggaattttttgtgtctctcactcggcctaggta |
| | | | gataagtagcatggcgggttaatcattaactacaaggaacccctagtgatggagttgg |
| | | | ccactccctctctgcgcgctcgctcgctcactgaggccgggcgaccaaaggtcgcccg |
| | | | acgcccgggctttgcccgggcggcctcagtgagcgagcgagcgcgcag |
| HER-05 | 3986 | 5170 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcgggcgggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaCcTTCaCCgacTATACCAT |
| | | | GGATtgggtgcgtcaggccccgggtaagggcctggaatgggttgcaGACGTGAATcct |
| | | | AATTCTggtGGTTCTATAtatAATCAGCGTTTCaagggccgtttcactCTGagcGTTg |
| | | | acCGTtccaaaaacacaCTGtacctgcagatgaacagcctgcgtgctgaggacactgc |
| | | | cgtctattattgtGCAagaAATCTGgggCCTAGCttctatTTCgacTACtggggtcaa |
| | | | ggaaccctggtcaccgtctcctcggcgtcgaccaagggcccatcggtcttccccctgg |
| | | | caccctcctccaagagcacctctgggggcacagcggccctgggctgcctggtcaagga |
| | | | ctacttccccgaacctgtgacggtctcgtggaactcaggcgccctgaccagcggcgtg |
| | | | cacaccttcccggctgtcctacagtcctcaggactctactccctcagcagcgtggtga |
| | | | ccgtgccctccagcagcttgggcacccagacctacatctgcaacgtgaatcacaagcc |
| | | | cagcaacaccaaggtggacaagaaagttgagcccaaatcttgtgacaaaactcacaca |
| | | | tgcccaccgtgcccagcacctgaactcctggggggaccgtcagtcttcctcttccccc |
| | | | caaaacccaaggacaccctcatgatctcccggacccctgaggtcacatgcgtggtggt |
| | | | ggacgtgagccacgaagaccctgaggtcaagttcaactggtacgtggacggcgtggag |
| | | | gtgcataatgccaagacaaagccgcgggaggagcagtacaacagcacgtaccgtgtgg |
| | | | tcagcgtcctcaccgtcctgcaccaggactggctgaatggcaaggagtacaagtgcaa |
| | | | ggtctccaacaaagccctcccagcccccatcgagaaaaccatctccaaagccaaaggg |
| | | | cagccccgagaaccacaggtgtacaccctgcccccatcccgggaGgagAtgaccaaga |
| | | | accaggtcagcctgacctgcctggtcaaaggcttctatcccagcgacatcgccgtgga |
| | | | gtgggagagcaatgggcagccggagaacaactacaagaccacgcctcccgtgctggac |
| | | | tccgacggctccttcttcctctacagcaagctcaccgtggacaagagcaggtggcagc |
| | | | aggggaacgtcttctcatgctccgtgatgcatgaggctctgcacaaccactacacgca |
| | | | gaagagcctctccctgtctccgggtaaaagaaagaggcgagagggcagaggaagtctt |
| | | | ctaacatgcggtgacgtggaggagaatcccggccctATGGACATGAGGGTCCCTGCTC |
| | | | AGCTCCTGGGGCTCCTGCTGCTCTGGCTCTCAGGTGCCAGATGTgatatccagatgac |
| | | | ccagtccccgagctccctgtccgcctctgtgggcgatagggtcaccatcacctgcAAA |
| | | | gccagtcaggatgtgTCCATTGGGgtagcctggtatcaacagaaaccaggaaaagctc |
| | | | cgaaactactgatttactcggcatccTATCGCTACACGggagtcccttctcgcttctc |
| | | | tggatccGGTtctgggacggatttcactctgaccatcagcagtctgcagccggaagac |
| | | | ttcgcaacttattactgtcagcaaTACtatATCTATcctTACacgttcggacagggta |
| | | | ccaaggtggagatcaaacgaactgtggctgcaccatctgtcttcatcttcccgccatc |
| | | | tgatgagcagttgaaatctggaactgcctctgttgtgtgcctgctgaataacttctat |
| | | | cccagagaggccaaagtacagtggaaggtggataacgccctccaatcgggtaactccc |
| | | | aggagagtgtcacagagcaggacagcaaggacagcacctacagcctcagcagcaccct |
| | | | gacgctgagcaaagcagactacgagaaacacaaagtctacgcctgcgaagtcacccat |
| | | | cagggcctgagctcgcccgtcacaaagagcttcaacaggggagagtgttagtagtaac |
| | | | tcgaggacggggtgaactacgcctgaggatccgatctttttccctctgccaaaaatta |
| | | | tggggacatcatgaagccccttgagcatctgacttctggctaataaaggaaatttatt |
| | | | ttcattgcaatagtgtgttggaattttttgtgtctctcactcggcctaggtagataag |
| | | | tagcatgggggttaatcattaactacaaggaacccctagtgatggagttggccactc |
| | | | cctctctgcgcgctcgctcgctcactgaggccgggcgaccaaaggtcgcccgacgccc |
| | | | gggctttgcccgggggcctcagtgagcgagcgagcgcgcag |
| HER-10 | 3989 | 5164 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcGACATTGATTATTGACTAGTTATTAATAG |
| | | | TAATCAATTACGGGGTCATTAGTTCATAGCCCATATATGGAGTTCCGCGTTACATAAC |
| | | | TTACGGTAAATGGCCCGCCTGGCTGACCGCCCAACGACCCCCGCCCATTGACGTCAAT |
| | | | AATGACGTATGTTCCCATAGTAACGCCAATAGGGACTTTCCATTGACGTCAATGGGTG |
| | | | GAGTATTTACGGTAAACTGCCCACTTGGCAGTACATCAAGTGTATCATATGCCAAGTA |
| | | | CGCCCCCTATTGACGTCAATGACGGTAAATGGCCCGCCTGGCATTATGCCCAGTACAT |
| | | | GACCTTATGGGACTTTCCTACTTGGCAGTACATCTACGTATTAGTCATCGCTATTACC |
| | | | ATGtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcgggcgggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAGGTGCAGCTGCAGCAGAGCGGCCCCGAGCTGGTGAAGCCCG |
| | | | GCGCCAGCCTGAAGCTGAGCTGCACCGCCAGCGGCTTCAACATCAAGGACACCTACAT |
| | | | CCACTGGGTGAAGCAGAGACCCGAGCAGGGCCTGGAGTGGATCGGCAGAATCTACCCC |
| | | | ACCAACGGCTACACCAGATACGACCCCAAGTTCCAGGACAAGGCCACCATCACCGCCG |
| | | | ACACCAGCAGCAACACCGCCTACCTGCAGGTGAGCAGACTGACCAGCGAGGACACCGC |
| | | | CGTGTACTACTGCAGCAGATGGGGCGGCGACGGCTTCTACGCCATGGACTACTGGGGC |
| | | | CAGGGCGCCAGCGTGACCGTGAGCAGCgcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaGagttgagcccaaatcttgtgacaaaactcac |
| | | | acatgcccaccgtgcccagcacctgaactcGtggggggaccgtcagtcttcctcCtcc |
| | | | ccccaaaacccaaggacaccctcatgatctcccggacccctgaggtcacatgcgtggt |
| | | | ggtggacgtgagccacgaagaccctgaggtcaagttcaactggtacgtggacggcgtg |
| | | | gaggtgcataatgccaagacaaagccgcCggaggagcagtacaacagcacgCTccgtg |
| | | | tggtcagcgtcctcaccgtcctgcaccaggactggctgaatggcaaggagtacaagtg |
| | | | caaggtctccaacaaagccctcccagcccccatcgagaaaaccatctccaaagccaaa |
| | | | gggcagccccgagaaccacaggtgtacaccctgcccccatcccgggaTgagCtgacca |
| | | | agaaccaggtcagcctgacctgcctggtcaaaggcttctatcccagcgacatcgccgt |
| | | | ggagtgggagagcaatgggcagccggagaacaactacaagaccacgcctcTcgtgctg |
| | | | gactccgacggctccttcttcctctacagcaagctcaccgtggacaagagcaggtggc |
| | | | agcaggggaacgtcttctcatgctccgtgatgcatgaggctctgcacaaccactacac |
| | | | gcagaagagcctctccctgtctccgggtaaaagaaagaggcgagagggcagaggaagt |
| | | | cttctaacatgcggtgacgtggaggagaatcccggccctATGGACATGAGGGTCCCTG |
| | | | CTCAGCTCCTGGGGCTCCTGCTGCTCTGGCTCTCAGGTGCCAGATGTGACATCGTGAT |
| | | | GACCCAGAGCCACAAGTTCATGAGCACCAGCGTGGGCGACAGAGTGAGCATCACCTGC |
| | | | AAGGCCAGCCAGGACGTGAACACCGCCGTGGCCTGGTACCAGCAGAAGCCCGGCCACA |
| | | | GCCCCAAGCTGCTGATCTACAGCGCCAGCTTCAGATACACCGGCGTGCCGGACAGATT |
| | | | CACCGGCAGCAGAAGCGGCACCGACTTCACCTTCACCATCAGCAGCGTGCAGGCCGAG |
| | | | GACCTGGCCGTGTACTACTGCCAGCAGCACTACACCACCCCTCCTACCTTCGGCGGCG |
| | | | GCACCAAGGTGGAGATCAAGAGAACCGTGGCCGCCCCTAGCGTGTTCATCTTCCCGCC |
| | | | AAGCGACGAGCAGCTGAAGAGCGGCACCGCCAGCGTGGTGTGCCTGCTGAACAACTTC |
| | | | TACCCCAGAGAGGCCAAGGTGCAGTGGAAGGTGGACAACGCCCTGCAGAGCGGCAACA |
| | | | GCCAGGAGAGCGTGACCGAGCAGGACAGCAAGGACAGCACCTACAGCCTGAGCAGCAC |
| | | | CCTGACCCTGAGCAAGGCCGACTACGAGAAGCACAAGGTGTACGCCTGCGAGGTGACC |
| | | | CACCAGGGCCTGAGCAGCCCCGTGACCAAGAGCTTCAACAGAGGCGAGTGCtagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggggcctcagtgagcgagcgagcgcgcag |
| HER-15 | 4220 | 5165 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcGACATTGATTATTGACTAGTTATTAATAG |
| | | | TAATCAATTACGGGGTCATTAGTTCATAGCCCATATATGGAGTTCCGCGTTACATAAC |
| | | | TTACGGTAAATGGCCCGCCTGGCTGACCGCCCAACGACCCCCGCCCATTGACGTCAAT |
| | | | AATGACGTATGTTCCCATAGTAACGCCAATAGGGACTTTCCATTGACGTCAATGGGTG |
| | | | GAGTATTTACGGTAAACTGCCCACTTGGCAGTACATCAAGTGTATCATATGCCAAGTA |
| | | | CGCCCCCTATTGACGTCAATGACGGTAAATGGCCCGCCTGGCATTATGCCCAGTACAT |
| | | | GACCTTATGGGACTTTCCTACTTGGCAGTACATCTACGTATTAGTCATCGCTATTACC |
| | | | ATGtcgaggccacgttctgcttcactctccccatctcccccccctccccacccccaat |
| | | | tttgtatttatttattttttaattattttgtgcagcgatgggggcggggggggggggc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaCcTTCaCCgacTATACCAT |
| | | | GGATtgggtgcgtcaggccccgggtaagggcctggaatgggttgcaGACGTGAATcct |
| | | | AATTCTggtGGTTCTATAtatAATCAGCGTTTCaagggccgtttcactCTGagcGTTg |
| | | | acCGTtccaaaaacacaCTGtacctgcagatgaacagcctgcgtgctgaggacactgc |
| | | | cgtctattattgtGCAagaAATCTGgggCCTAGCttctatTTCgacTACtggggtcaa |
| | | | ggaaccctggtcaccgtctcctcggcgtcgaccaagggcccatcggtcttccccctgg |
| | | | caccctcctccaagagcacctctgggggcacagcggccctgggctgcctggtcaagga |
| | | | ctacttccccgaacctgtgacggtctcgtggaactcaggcgccctgaccagcggcgtg |
| | | | cacaccttcccggctgtcctacagtcctcaggactctactccctcagcagcgtggtga |
| | | | ccgtgccctccagcagcttgggcacccagacctacatctgcaacgtgaatcacaagcc |
| | | | cagcaacaccaaggtggacaagaaagttgagcccaaatcttgtgacaaaactcacaca |
| | | | tgcccaccgtgcccagcacctgaactcctggggggaccgtcagtcttcctcttccccc |
| | | | caaaacccaaggacaccctcatgatctcccggacccctgaggtcacatgcgtggtggt |
| | | | ggacgtgagccacgaagaccctgaggtcaagttcaactggtacgtggacggcgtggag |
| | | | gtgcataatgccaagacaaagccgcgggaggagcagtacaacagcacgtaccgtgtgg |
| | | | tcagcgtcctcaccgtcctgcaccaggactggctgaatggcaaggagtacaagtgcaa |
| | | | ggtctccaacaaagccctcccagcccccatcgagaaaaccatctccaaagccaaaggg |
| | | | cagccccgagaaccacaggtgtacaccctgcccccatcccgggaGgagAtgaccaaga |
| | | | accaggtcagcctgacctgcctggtcaaaggcttctatcccagcgacatcgccgtgga |
| | | | gtgggagagcaatgggcagccggagaacaactacaagaccacgcctcccgtgctggac |
| | | | tccgacggctccttcttcctctacagcaagctcaccgtggacaagagcaggtggcagc |
| | | | aggggaacgtcttctcatgctccgtgatgcatgaggctctgcacaaccactacacgca |
| | | | gaagagcctctccctgtctccgggtaaaagaaagaggcgagagggcagaggaagtctt |
| | | | ctaacatgcggtgacgtggaggagaatcccggccctATGGACATGAGGGTCCCTGCTC |
| | | | AGCTCCTGGGGCTCCTGCTGCTCTGGCTCTCAGGTGCCAGATGTGGCGTGACCCTGTT |
| | | | CGTGGCCCTGTACGACTACACCAGCTACAACACCAGGGACCTGAGCTTCCACAAGGGC |
| | | | GAGAAGTTCCAGATCCTGAGGATGGAGGACGGCGTGTGGTGGGAGGCCAGGAGCCTGA |
| | | | CCACCGGCGAGACCGGCTACATCCCCAGCAACTACGTGGCCCCCGTGGACAGCATCCA |
| | | | GggtggtggtggatccggaggaggaggaAGTggaggtggaggtTCTgatatccagatg |
| | | | acccagtccccgagctccctgtccgcctctgtgggcgatagggtcaccatcacctgcA |
| | | | AAgccagtcaggatgtgTCCATTGGGgtagcctggtatcaacagaaaccaggaaaagc |
| | | | tccgaaactactgatttactcggcatccTATCGCTACACGggagtcccttctcgcttc |
| | | | tctggatccGGTtctgggacggatttcactctgaccatcagcagtctgcagccggaag |
| | | | acttcgcaacttattactgtcagcaaTACtatATCTATcctTACacgttcggacaggg |
| | | | taccaaggtggagatcaaacgaactgtggctgcaccatctgtcttcatcttcccgcca |
| | | | tctgatgagcagttgaaatctggaactgcctctgttgtgtgcctgctgaataacttct |
| | | | atcccagagaggccaaagtacagtggaaggtggataacgccctccaatcgggtaactc |
| | | | ccaggagagtgtcacagagcaggacagcaaggacagcacctacagcctcagcagcacc |
| | | | ctgacgctgagcaaagcagactacgagaaacacaaagtctacgcctgcgaagtcaccc |
| | | | atcagggcctgagctcgcccgtcacaaagagcttcaacaggggagagtgttagtagta |
| | | | actcgaggacggggtgaactacgcctgaggatccgatctttttccctctgccaaaaat |
| | | | tatggggacatcatgaagccccttgagcatctgacttctggctaataaaggaaattta |
| | | | ttttcattgcaatagtgtgttggaattttttgtgtctctcactcggcctaggtagata |
| | | | agtagcatggcgggttaatcattaactacaaggaacccctagtgatggagttggccac |
| | | | tccctctctgcgcgctcgctcgctcactgaggccgggcgaccaaaggtcgcccgacgc |
| | | | ccgggctttgcccgggcggcctcagtgagcgagcgagcgcgcag |
| HER-42 | 3992 | 5166 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcGACATTGATTATTGACTAGTTATTAATAG |
| | | | TAATCAATTACGGGGTCATTAGTTCATAGCCCATATATGGAGTTCCGCGTTACATAAC |
| | | | TTACGGTAAATGGCCCGCCTGGCTGACCGCCCAACGACCCCCGCCCATTGACGTCAAT |
| | | | AATGACGTATGTTCCCATAGTAACGCCAATAGGGACTTTCCATTGACGTCAATGGGTG |
| | | | GAGTATTTACGGTAAACTGCCCACTTGGCAGTACATCAAGTGTATCATATGCCAAGTA |
| | | | CGCCCCCTATTGACGTCAATGACGGTAAATGGCCCGCCTGGCATTATGCCCAGTACAT |
| | | | GACCTTATGGGACTTTCCTACTTGGCAGTACATCTACGTATTAGTCATCGCTATTACC |
| | | | ATGtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaaagttgagcccCCCaaatcttgtgacaaaact |
| | | | cacacatgcccaccgtgcccagcacctgaactcctggggggaccgtcagtcttcctct |
| | | | tccccccaaaacccaaggacaccctcatgatctcccggacccctgaggtcacatgcgt |
| | | | ggtggtggacgtgagccacgaagaccctgaggtcaagttcaactggtacgtggacggc |
| | | | gtggaggtgcataatgccaagacaaagccgcgggaggagcagtacaacagcacgtacc |
| | | | gtgtggtcagcgtcctcaccgtcctgcaccaggactggctgaatggcaaggagtacaa |
| | | | gtgcaaggtctccaacaaagccctcccagcccccatcgagaaaaccatctccaaagcc |
| | | | aaagggcagccccgagaaccacaggtgtacaccctgcccccatcccgggatgagctga |
| | | | ccaagaaccaggtcagcctgacctgcctggtcaaaggcttctatcccagcgacatcgc |
| | | | cgtggagtgggagagcaatgggcagccggagaacaactacaagaccacgcctcccgtg |
| | | | ctggactccgacggctccttcttcctctacagcaagctcaccgtggacaagagcaggt |
| | | | ggcagcaggggaacgtcttctcatgctccgtgatgcatgaggctctgcacaacCAGta |
| | | | cacgcagaagagcctctccctgtctccgggtaaaAgaaagaggcgagagggcagagga |
| | | | agtcttctaacatgcggtgacgtggaggagaatcccggccctATGGACATGAGGGTCC |
| | | | CTGCTCAGCTCCTGGGGCTCCTGCTGCTCTGGCTCTCAGGTGCCAGATGTgatatcca |
| | | | gatgacccagtccccgagctccctgtccgcctctgtgggcgatagggtcaccatcacc |
| | | | tgccgtgccagtcaggatgtgaatactgctgtagcctggtatcaacagaaaccaggaa |
| | | | aagctccgaaactactgatttactcggcatccttcctcTACtctggagtcccttctcg |
| | | | cttctctggatccagatctgggacggatttcactctgaccatcagcagtctgcagccg |
| | | | gaagacttcgcaacttattactgtcagcaacattatactactcctcccacgttcggac |
| | | | agggtaccaaggtggagatcaaacgaactgtggctgcaccatctgtcttcatcttccc |
| | | | gccatctgatgagcagttgaaatctggaactgcctctgttgtgtgcctgctgaataac |
| | | | ttctatcccagagaggccaaagtacagtggaaggtggataacgccctccaatcgggta |
| | | | actcccaggagagtgtcacagagcaggacagcaaggacagcacctacagcctcagcag |
| | | | caccctgacgctgagcaaagcagactacgagaaacacaaagtctacgcctgcgaagtc |
| | | | acccatcagggcctgagctcgcccgtcacaaagagcttcaacaggggagagtgttagt |
| | | | agtaactcgaggacggggtgaactacgcctgaggatccgatctttttccctctgccaa |
| | | | aaattatggggacatcatgaagccccttgagcatctgacttctggctaataaaggaaa |
| | | | tttattttcattgcaatagtgtgttggaattttttgtgtctctcactcggcctaggta |
| | | | gataagtagcatggcgggttaatcattaactacaaggaacccctagtgatggagttgg |
| | | | ccactccctctctgcgcgctcgctcgctcactgaggccgggcgaccaaaggtcgcccg |
| | | | acgcccgggctttgcccgggcggcctcagtgagcgagcgagcgcgcag |
| HER-43 | 3992 | 5185 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaaagttgagcccCCCaaatcttgtgacaaaact |
| | | | cacacatgcccaccgtgcccagcacctgaactcctggggggaccgtcagtcttcctct |
| | | | tccccccaaaacccaaggacaccctcatgGCAtcccggacccctgaggtcacatgcgt |
| | | | ggtggtggacgtgagccacgaagaccctgaggtcaagttcaactggtacgtggacggc |
| | | | gtggaggtgcataatgccaagacaaagccgcgggaggagcagtacaacagcacgtacc |
| | | | gtgtggtcagcgtcctcaccgtcctgGCCcaggactggctgaatggcaaggagtacaa |
| | | | gtgcaaggtctccaacaaagccctcccagcccccatcgagaaaaccatctccaaagcc |
| | | | aaagggcagccccgagaaccacaggtgtacaccctgcccccatcccgggatgagctga |
| | | | ccaagaaccaggtcagcctgacctgcctggtcaaaggcttctatcccagcgacatcgc |
| | | | cgtggagtgggagagcaatgggcagccggagaacaactacaagaccacgcctcccgtg |
| | | | ctggactccgacggctccttcttcctctacagcaagctcaccgtggacaagagcaggt |
| | | | ggcagcaggggaacgtcttctcatgctccgtgatgcatgaggctctgcacaaccacta |
| | | | cacgcagaagagcctctccctgtctccgggtaaaagaaagaggcgagagggcagagga |
| | | | agtcttctaacatgcggtgacgtggaggagaatcccggccctATGGACATGAGGGTCC |
| | | | CTGCTCAGCTCCTGGGGCTCCTGCTGCTCTGGCTCTCAGGTGCCAGATGTgatatcca |
| | | | gatgacccagtccccgagctccctgtccgcctctgtgggcgatagggtcaccatcacc |
| | | | tgccgtgccagtcaggatgtgaatactgctgtagcctggtatcaacagaaaccaggaa |
| | | | aagctccgaaactactgatttactcggcatccttcctcTACtctggagtcccttctcg |
| | | | cttctctggatccagatctgggacggatttcactctgaccatcagcagtctgcagccg |
| | | | gaagacttcgcaacttattactgtcagcaacattatactactcctcccacgttcggac |
| | | | agggtaccaaggtggagatcaaacgaactgtggctgcaccatctgtcttcatcttccc |
| | | | gccatctgatgagcagttgaaatctggaactgcctctgttgtgtgcctgctgaataac |
| | | | ttctatcccagagaggccaaagtacagtggaaggtggataacgccctccaatcgggta |
| | | | actcccaggagagtgtcacagagcaggacagcaaggacagcacctacagcctcagcag |
| | | | caccctgacgctgagcaaagcagactacgagaaacacaaagtctacgcctgcgaagtc |
| | | | acccatcagggcctgagctcgcccgtcacaaagagcttcaacaggggagagtgttagt |
| | | | agtaactcgaggacggggtgaactacgcctgaggatccgatctttttccctctgccaa |
| | | | aaattatggggacatcatgaagccccttgagcatctgacttctggctaataaaggaaa |
| | | | tttattttcattgcaatagtgtgttggaattttttgtgtctctcactcggcctaggta |
| | | | gataagtagcatggcgggttaatcattaactacaaggaacccctagtgatggagttgg |
| | | | ccactccctctctgcgcgctcgctcgctcactgaggccgggcgaccaaaggtcgcccg |
| | | | acgcccgggctttgcccgggcggcctcagtgagcgagcgagcgcgcag |
| HER-46 | 3992 | 5186 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaaagttgagcccCCCaaatcttgtgacaaaact |
| | | | cacacatgcccaccgtgcccagcacctgaactcctggggggaccgtcagtcttcctct |
| | | | tccccccaaaacccaaggacaccctcatgGCAtcccggacccctgaggtcacatgcgt |
| | | | ggtggtggacgtgagccacgaagaccctgaggtcaagttcaactggtacgtggacggc |
| | | | gtggaggtgcataatgccaagacaaagccgcgggaggagcagtacaacagcacgtacc |
| | | | gtgtggtcagcgtcctcaccgtcctgcaccaggactggctgaatggcaaggagtacaa |
| | | | gtgcaaggtctccaacaaagccctcccagcccccatcgagaaaaccatctccaaagcc |
| | | | aaagggcagccccgagaaccacaggtgtacaccctgcccccatcccgggatgagctga |
| | | | ccaagaaccaggtcagcctgacctgcctggtcaaaggcttctatcccagcgacatcgc |
| | | | cgtggagtgggagagcaatgggcagccggagaacaactacaagaccacgcctcccgtg |
| | | | ctggactccgacggctccttcttcctctacagcaagctcaccgtggacaagagcaggt |
| | | | ggcagcaggggaacgtcttctcatgctccgtgatgcatgaggctctgcacaacCAGta |
| | | | cacgcagaagagcctctccctgtctccgggtaaaagaaagaggcgagagggcagagga |
| | | | agtcttctaacatgcggtgacgtggaggagaatcccggccctATGGACATGAGGGTCC |
| | | | CTGCTCAGCTCCTGGGGCTCCTGCTGCTCTGGCTCTCAGGTGCCAGATGTgatatcca |
| | | | gatgacccagtccccgagctccctgtccgcctctgtgggcgatagggtcaccatcacc |
| | | | tgccgtgccagtcaggatgtgaatactgctgtagcctggtatcaacagaaaccaggaa |
| | | | aagctccgaaactactgatttactcggcatccttcctcTACtctggagtcccttctcg |
| | | | cttctctggatccagatctgggacggatttcactctgaccatcagcagtctgcagccg |
| | | | gaagacttcgcaacttattactgtcagcaacattatactactcctcccacgttcggac |
| | | | agggtaccaaggtggagatcaaacgaactgtggctgcaccatctgtcttcatcttccc |
| | | | gccatctgatgagcagttgaaatctggaactgcctctgttgtgtgcctgctgaataac |
| | | | ttctatcccagagaggccaaagtacagtggaaggtggataacgccctccaatcgggta |
| | | | actcccaggagagtgtcacagagcaggacagcaaggacagcacctacagcctcagcag |
| | | | caccctgacgctgagcaaagcagactacgagaaacacaaagtctacgcctgcgaagtc |
| | | | acccatcagggcctgagctcgcccgtcacaaagagcttcaacaggggagagtgttagt |
| | | | agtaactcgaggacggggtgaactacgcctgaggatccgatctttttccctctgccaa |
| | | | aaattatggggacatcatgaagccccttgagcatctgacttctggctaataaaggaaa |
| | | | tttattttcattgcaatagtgtgttggaattttttgtgtctctcactcggcctaggta |
| | | | gataagtagcatggcgggttaatcattaactacaaggaacccctagtgatggagttgg |
| | | | ccactccctctctgcgcgctcgctcgctcactgaggccgggcgaccaaaggtcgcccg |
| | | | acgcccgggctttgcccgggcggcctcagtgagcgagcgagcgcgcag |
| HER-47 | 3992 | 5167 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcGACATTGATTATTGACTAGTTATTAATAG |
| | | | TAATCAATTACGGGGTCATTAGTTCATAGCCCATATATGGAGTTCCGCGTTACATAAC |
| | | | TTACGGTAAATGGCCCGCCTGGCTGACCGCCCAACGACCCCCGCCCATTGACGTCAAT |
| | | | AATGACGTATGTTCCCATAGTAACGCCAATAGGGACTTTCCATTGACGTCAATGGGTG |
| | | | GAGTATTTACGGTAAACTGCCCACTTGGCAGTACATCAAGTGTATCATATGCCAAGTA |
| | | | CGCCCCCTATTGACGTCAATGACGGTAAATGGCCCGCCTGGCATTATGCCCAGTACAT |
| | | | GACCTTATGGGACTTTCCTACTTGGCAGTACATCTACGTATTAGTCATCGCTATTACC |
| | | | ATGtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatactttttttgttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaaagttgagcccCCCaaatcttgtgacaaaact |
| | | | cacacatgcccaccgtgcccagcacctgaactcctggggggaccgtcagtcttcctct |
| | | | tccccccaaaacccaaggacaccctcatgGCAtcccggacccctgaggtcacatgcgt |
| | | | ggtggtggacgtgagccacgaagaccctgaggtcaagttcaactggtacgtggacggc |
| | | | gtggaggtgcataatgccaagacaaagccgcgggaggagcagtacaacagcacgtacc |
| | | | gtgtggtcagcgtcctcaccgtcctgGCCcaggactggctgaatggcaaggagtacaa |
| | | | gtgcaaggtctccaacaaagccctcccagcccccatcgagaaaaccatctccaaagcc |
| | | | aaagggcagccccgagaaccacaggtgtacaccctgcccccatcccgggatgagctga |
| | | | ccaagaaccaggtcagcctgacctgcctggtcaaaggcttctatcccagcgacatcgc |
| | | | cgtggagtgggagagcaatgggcagccggagaacaactacaagaccacgcctcccgtg |
| | | | ctggactccgacggctccttcttcctctacagcaagctcaccgtggacaagagcaggt |
| | | | ggcagcaggggaacgtcttctcatgctccgtgatgcatgaggctctgcacaacGCTta |
| | | | cacgcagaagagcctctccctgtctccgggtaaaAgaaagaggcgagagggcagagga |
| | | | agtcttctaacatgcggtgacgtggaggagaatcccggccctATGGACATGAGGGTCC |
| | | | CTGCTCAGCTCCTGGGGCTCCTGCTGCTCTGGCTCTCAGGTGCCAGATGTgatatcca |
| | | | gatgacccagtccccgagctccctgtccgcctctgtgggcgatagggtcaccatcacc |
| | | | tgccgtgccagtcaggatgtgaatactgctgtagcctggtatcaacagaaaccaggaa |
| | | | aagctccgaaactactgatttactcggcatccttcctcTACtctggagtcccttctcg |
| | | | cttctctggatccagatctgggacggatttcactctgaccatcagcagtctgcagccg |
| | | | gaagacttcgcaacttattactgtcagcaacattatactactcctcccacgttcggac |
| | | | agggtaccaaggtggagatcaaacgaactgtggctgcaccatctgtcttcatcttccc |
| | | | gccatctgatgagcagttgaaatctggaactgcctctgttgtgtgcctgctgaataac |
| | | | ttctatcccagagaggccaaagtacagtggaaggtggataacgccctccaatcgggta |
| | | | actcccaggagagtgtcacagagcaggacagcaaggacagcacctacagcctcagcag |
| | | | caccctgacgctgagcaaagcagactacgagaaacacaaagtctacgcctgcgaagtc |
| | | | acccatcagggcctgagctcgcccgtcacaaagagcttcaacaggggagagtgttagt |
| | | | agtaactcgaggacggggtgaactacgcctgaggatccgatctttttccctctgccaa |
| | | | aaattatggggacatcatgaagccccttgagcatctgacttctggctaataaaggaaa |
| | | | tttattttcattgcaatagtgtgttggaattttttgtgtctctcactcggcctaggta |
| | | | gataagtagcatggcgggttaatcattaactacaaggaacccctagtgatggagttgg |
| | | | ccactccctctctgcgcgctcgctcgctcactgaggccgggcgaccaaaggtcgcccg |
| | | | acgcccgggctttgcccgggcggcctcagtgagcgagcgagcgcgcag |
| HER-53 | 3989 | 5168 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcGACATTGATTATTGACTAGTTATTAATAG |
| | | | TAATCAATTACGGGGTCATTAGTTCATAGCCCATATATGGAGTTCCGCGTTACATAAC |
| | | | TTACGGTAAATGGCCCGCCTGGCTGACCGCCCAACGACCCCCGCCCATTGACGTCAAT |
| | | | AATGACGTATGTTCCCATAGTAACGCCAATAGGGACTTTCCATTGACGTCAATGGGTG |
| | | | GAGTATTTACGGTAAACTGCCCACTTGGCAGTACATCAAGTGTATCATATGCCAAGTA |
| | | | CGCCCCCTATTGACGTCAATGACGGTAAATGGCCCGCCTGGCATTATGCCCAGTACAT |
| | | | GACCTTATGGGACTTTCCTACTTGGCAGTACATCTACGTATTAGTCATCGCTATTACC |
| | | | ATGtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaGagttgagcccaaatcttgtgacaaaactcac |
| | | | acatgcccaccgtgcccagcacctgaactcGtggggggaccgtcagtcttcctcCtcc |
| | | | ccccaaaacccaaggacaccctcatgatctcccggacccctgaggtcacatgcgtggt |
| | | | ggtggacgtgagccacgaagaccctgaggtcaagttcaactggtacgtggacggcgtg |
| | | | gaggtgcataatgccaagacaaagccgcCggaggagcagtacaacagcacgCTccgtg |
| | | | tggtcagcgtcctcaccgtcctgcaccaggactggctgaatggcaaggagtacaagtg |
| | | | caaggtctccaacaaagccctcccagcccccatcgagaaaaccatctccaaagccaaa |
| | | | gggcagccccgagaaccacaggtgtacaccctgcccccatcccgggaTgagCtgacca |
| | | | agaaccaggtcagcctgacctgcctggtcaaaggcttctatcccagcgacatcgccgt |
| | | | ggagtgggagagcaatgggcagccggagaacaactacaagaccacgcctcTcgtgctg |
| | | | gactccgacggctccttcttcctctacagcaagctcaccgtggacaagagcaggtggc |
| | | | agcaggggaacgtcttctcatgctccgtgatgcatgaggctctgcacaaccactacac |
| | | | gcagaagagcctctccctgtctccgggtaaaAgaaagaggcgagagggcagaggaagt |
| | | | cttctaacatgcggtgacgtggaggagaatcccggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccgagctccctgtccgcctctgtgggcgatagggtcaccatcacctgc |
| | | | cgtgccagtcaggatgtgaatactgctgtagcctggtatcaacagaaaccaggaaaag |
| | | | ctccgaaactactgatttactcggcatccttcctcTACtctggagtcccttctcgctt |
| | | | ctctggatccagatctgggacggatttcactctgaccatcagcagtctgcagccggaa |
| | | | gacttcgcaacttattactgtcagcaacattatactactcctcccacgttcggacagg |
| | | | gtaccaaggtggagatcaaacgaactgtggctgcaccatctgtcttcatcttcccgcc |
| | | | atctgatgagcagttgaaatctggaactgcctctgttgtgtgcctgctgaataacttc |
| | | | tatcccagagaggccaaagtacagtggaaggtggataacgccctccaatcgggtaact |
| | | | cccaggagagtgtcacagagcaggacagcaaggacagcacctacagcctcagcagcac |
| | | | cctgacgctgagcaaagcagactacgagaaacacaaagtctacgcctgcgaagtcacc |
| | | | catcagggcctgagctcgcccgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-57 | 3287 | 5187 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcgggtggtggtggatccggaggaggaggaAGTg |
| | | | gaggtggaggtTCTgatatccagatgacccagtccccgagctccctgtccgcctctgt |
| | | | gggcgatagggtcaccatcacctgccgtgccagtcaggatgtgaatactgctgtagcc |
| | | | tggtatcaacagaaaccaggaaaagctccgaaactactgatttactcggcatccttcc |
| | | | tcTACtctggagtcccttctcgcttctctggatccagatctgggacggatttcactct |
| | | | gaccatcagcagtctgcagccggaagacttcgcaacttattactgtcagcaacattat |
| | | | actactcctcccacgttcggacagggtaccaaggtggagatcaaagagcccaaatctt |
| | | | gtgacaaaactTACacatgcccaccgtgcccagcacctgaactcGtggggggaccgtc |
| | | | agtcttcctcCtccccccaaaacccaaggacaccctcatgatctcccggacccctgag |
| | | | gtcacatgcgtggtggtggacgtgagccacgaagaccctgaggtcaagttcaactggt |
| | | | acgtggacggcgtggaggtgcataatgccaagacaaagccgcCggaggagcagtacaa |
| | | | cagcacgCTccgtgtggtcagcgtcctcaccgtcctgcaccaggactggctgaatggc |
| | | | aaggagtacaagtgcaaggtctccaacaaagccctcccagcccccatcgagaaaacca |
| | | | tctccaaagccaaagggcagccccgagaaccacaggtgtacaccctgcccccatcccg |
| | | | ggaTgagCtgaccaagaaccaggtcagcctgacctgcctggtcaaaggcttctatccc |
| | | | agcgacatcgccgtggagtgggagagcaatgggcagccggagaacaactacaagacca |
| | | | cgcctcTcgtgctggactccgacggctccttcttcctctacagcaagctcaccgtgga |
| | | | caagagcaggtggcagcaggggaacgtcttctcatgctccgtgatgcatgaggctctg |
| | | | cacaaccactacacgcagaagagcctctccctgtctccgggtaaatagtagtaactcg |
| | | | aggacggggtgaactacgcctgaggatccgatctttttccctctgccaaaaattatgg |
| | | | ggacatcatgaagccccttgagcatctgacttctggctaataaaggaaatttattttc |
| | | | attgcaatagtgtgttggaattttttgtgtctctcactcggcctaggtagataagtag |
| | | | catggcgggttaatcattaactacaaggaacccctagtgatggagttggccactccct |
| | | | ctctgcgcgctcgctcgctcactgaggccgggcgaccaaaggtcgcccgacgcccggg |
| | | | ctttgcccgggcggcctcagtgagcgagcgagcgcgcag |
| HER-62 | 3284 | 5188 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaCcTTCaCCgacTATACCAT |
| | | | GGATtgggtgcgtcaggccccgggtaagggcctggaatgggttgcaGACGTGAATcct |
| | | | AATTCTggtGGTTCTATAtatAATCAGCGTTTCaagggccgtttcactCTGagcGTTg |
| | | | acCGTtccaaaaacacaCTGtacctgcagatgaacagcctgcgtgctgaggacactgc |
| | | | cgtctattattgtGCAagaAATCTGgggCCTAGCttctatTTCgacTACtggggtcaa |
| | | | ggaaccctggtcaccgtctcctcgggtggtggtggatccggaggaggaggaAGTggag |
| | | | gtggaggtTCTgatatccagatgacccagtccccgagctccctgtccgcctctgtggg |
| | | | cgatagggtcaccatcacctgcAAAgccagtcaggatgtgTCCATTGGGgtagcctgg |
| | | | tatcaacagaaaccaggaaaagctccgaaactactgatttactcggcatccTATCGCT |
| | | | ACACGggagtcccttctcgcttctctggatccGGTtctgggacggatttcactctgac |
| | | | catcagcagtctgcagccggaagacttcgcaacttattactgtcagcaaTACtatATC |
| | | | TATcctTACacgttcggacagggtaccaaggtggagatcaaagagcccaaatcttgtg |
| | | | acaaaactTACacatgcccaccgtgcccagcacctgaactcGtggggggaccgtcagt |
| | | | cttcctcCtccccccaaaacccaaggacaccctcatgatctcccggacccctgaggtc |
| | | | acatgcgtggtggtggacgtgagccacgaagaccctgaggtcaagttcaactggtacg |
| | | | tggacggcgtggaggtgcataatgccaagacaaagccgcCggaggagcagtacaacag |
| | | | cacgCTccgtgtggtcagcgtcctcaccgtcctgcaccaggactggctgaatggcaag |
| | | | gagtacaagtgcaaggtctccaacaaagccctcccagcccccatcgagaaaaccatct |
| | | | ccaaagccaaagggcagccccgagaaccacaggtgtacaccctgcccccatcccggga |
| | | | TgagCtgaccaagaaccaggtcagcctgacctgcctggtcaaaggcttctatcccagc |
| | | | gacatcgccgtggagtgggagagcaatgggcagccggagaacaactacaagaccacgc |
| | | | ctcTcgtgctggactccgacggctccttcttcctctacagcaagctcaccgtggacaa |
| | | | gagcaggtggcagcaggggaacgtcttctcatgctccgtgatgcatgaggctctgcac |
| | | | aaccactacacgcagaagagcctctccctgtctccgggtaaatagtagtaactcgagg |
| | | | acggggtgaactacgcctgaggatccgatctttttccctctgccaaaaattatgggga |
| | | | catcatgaagccccttgagcatctgacttctggctaataaaggaaatttattttcatt |
| | | | gcaatagtgtgttggaattttttgtgtctctcactcggcctaggtagataagtagcat |
| | | | ggcgggttaatcattaactacaaggaacccctagtgatggagttggccactccctctc |
| | | | tgcgcgctcgctcgctcactgaggccgggcgaccaaaggtcgcccgacgcccgggctt |
| | | | tgcccgggcggcctcagtgagcgagcgagcgcgcag |
| HER-69 | 4047 | 5169 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacataacgcgtgatctaacatatcctgg |
| | | | tgtggagtagcggacgctgctatgacagaggctcgggggcctgagctggctctgtgag |
| | | | ctggggaggaggcagacagccaggccttgtctgcaagcagacctggcagcattgggct |
| | | | ggccgccccccagggcctcctcttcatgcccagtgaatgactcaccttggcacagaca |
| | | | caatgttcggggtgggcacagtgcctgcttcccgccgcaccccagcccccctcaaatg |
| | | | ccttccgagaagcccattgagcagggggcttgcattgcaccccagcctgacagcctgg |
| | | | catcttgggataaaagcagcacagccccctaggggctgcccttgctgtgtggcgccac |
| | | | cggcggtggagaacaaggctctattcagcctgtgcccaggaaaggggatcaggggatg |
| | | | cccaggcatggacagtgggtggcagggggggagaggagggctgtctgcttcccagaag |
| | | | tccaaggacacaaatgggtgaggggagagctctccccatagctgggctgcggcccaac |
| | | | cccaccccctcaggctatgccagggggtgttgccaggggcacccgggcatcgccagtc |
| | | | tagcccactccttcataaagccctcgcatcccaggagcgagcagagccagagcaggtt |
| | | | ggagaggagacgcatcacctccgctgctcgcggggatcctctagaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAGGTGCAGCTGCAGCAGAGCGGCCCCGAGCTGGTGAAGCCCG |
| | | | GCGCCAGCCTGAAGCTGAGCTGCACCGCCAGCGGCTTCAACATCAAGGACACCTACAT |
| | | | CCACTGGGTGAAGCAGAGACCCGAGCAGGGCCTGGAGTGGATCGGCAGAATCTACCCC |
| | | | ACCAACGGCTACACCAGATACGACCCCAAGTTCCAGGACAAGGCCACCATCACCGCCG |
| | | | ACACCAGCAGCAACACCGCCTACCTGCAGGTGAGCAGACTGACCAGCGAGGACACCGC |
| | | | CGTGTACTACTGCAGCAGATGGGGCGGCGACGGCTTCTACGCCATGGACTACTGGGGC |
| | | | CAGGGCGCCAGCGTGACCGTGAGCAGCgcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaGagttgagcccaaatcttgtgacaaaactcac |
| | | | acatgcccaccgtgcccagcacctgaactcGtggggggaccgtcagtcttcctcCtcc |
| | | | ccccaaaacccaaggacaccctcatgatctcccggacccctgaggtcacatgcgtggt |
| | | | ggtggacgtgagccacgaagaccctgaggtcaagttcaactggtacgtggacggcgtg |
| | | | gaggtgcataatgccaagacaaagccgcCggaggagcagtacaacagcacgCTccgtg |
| | | | tggtcagcgtcctcaccgtcctgcaccaggactggctgaatggcaaggagtacaagtg |
| | | | caaggtctccaacaaagccctcccagcccccatcgagaaaaccatctccaaagccaaa |
| | | | gggcagccccgagaaccacaggtgtacaccctgcccccatcccgggaTgagCtgacca |
| | | | agaaccaggtcagcctgacctgcctggtcaaaggcttctatcccagcgacatcgccgt |
| | | | ggagtgggagagcaatgggcagccggagaacaactacaagaccacgcctcTcgtgctg |
| | | | gactccgacggctccttcttcctctacagcaagctcaccgtggacaagagcaggtggc |
| | | | agcaggggaacgtcttctcatgctccgtgatgcatgaggctctgcacaaccactacac |
| | | | gcagaagagcctctccctgtctccgggtaaaagaaagaggcgagagggcagaggaagt |
| | | | cttctaacatgcggtgacgtggaggagaatcccggccctATGGACATGAGGGTCCCTG |
| | | | CTCAGCTCCTGGGGCTCCTGCTGCTCTGGCTCTCAGGTGCCAGATGTGACATCGTGAT |
| | | | GACCCAGAGCCACAAGTTCATGAGCACCAGCGTGGGCGACAGAGTGAGCATCACCTGC |
| | | | AAGGCCAGCCAGGACGTGAACACCGCCGTGGCCTGGTACCAGCAGAAGCCCGGCCACA |
| | | | GCCCCAAGCTGCTGATCTACAGCGCCAGCTTCAGATACACCGGCGTGCCGGACAGATT |
| | | | CACCGGCAGCAGAAGCGGCACCGACTTCACCTTCACCATCAGCAGCGTGCAGGCCGAG |
| | | | GACCTGGCCGTGTACTACTGCCAGCAGCACTACACCACCCCTCCTACCTTCGGCGGCG |
| | | | GCACCAAGGTGGAGATCAAGAGAACCGTGGCCGCCCCTAGCGTGTTCATCTTCCCGCC |
| | | | AAGCGACGAGCAGCTGAAGAGCGGCACCGCCAGCGTGGTGTGCCTGCTGAACAACTTC |
| | | | TACCCCAGAGAGGCCAAGGTGCAGTGGAAGGTGGACAACGCCCTGCAGAGCGGCAACA |
| | | | GCCAGGAGAGCGTGACCGAGCAGGACAGCAAGGACAGCACCTACAGCCTGAGCAGCAC |
| | | | CCTGACCCTGAGCAAGGCCGACTACGAGAAGCACAAGGTGTACGCCTGCGAGGTGACC |
| | | | CACCAGGGCCTGAGCAGCCCCGTGACCAAGAGCTTCAACAGAGGCGAGTGCtagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-73 | 4742 | 5189 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcAgccctgggctgcctggtcaa |
| | | | ggactacttcccTgaacctgtgacAgtctcTtggaactcaggGgccctgaccTCTggG |
| | | | gtgcacaccttcccAgctgtcctacagtcctcaggactctactccctcTCTTCTgtgg |
| | | | tgacAgtgccctccagcagcttgggcacccagacctacatctgcaaTgtgaatcacaa |
| | | | gcccagcaacaccaaggtggacaagaaagttgagcccCCCaaatcttgtgacaaaact |
| | | | cacacatgcccaccCtgcccagcacctgaactcctggggggaccCtcagtcttcctct |
| | | | tccccccaaaacccaaggacaccctcatgatctccAGAacccctgaggtcacatgTgt |
| | | | ggtggtggaTgtgagccaTgaagaccctgaggtcaagttcaactggtaTgtggaTggG |
| | | | gtggaggtgcataatgccaagacaaagccCAGAgaggagcagtacaacagcacCtacA |
| | | | GAgtggtcTCAgtcctcacTgtcctgcaccaggactggctgaatggcaaggagtacaa |
| | | | gtgcaaggtctccaacaaagccctcccagcccccatAgagaaaaccatctccaaagcc |
| | | | aaagggcagcccAGAgaaccacaggtgtacaccctgcccccatccAGAgatgagctga |
| | | | ccaagaaccaggtcagcctgacctgcctggtcaaaggcttctatcccTCTgacatTgc |
| | | | TgtggagtgggagagcaatgggcagccAgagaacaactacaagaccacCcctccAgtg |
| | | | ctggactcTgatggctccttcttcctctacagcaagctcacAgtggacaagagcaggt |
| | | | ggcagcaggggaaTgtcttctcatgctcTgtgatgcatgaggctctgcacaaccacta |
| | | | cacTcagaagagcctctccctgtctccTggtaaaagaaagaggAGAgagggcagagga |
| | | | agtcttctaacatgTggtgaTgtggaggagaatccTggccctATGGACATGAGGGTCC |
| | | | CTGCTCAGCTCCTGGGGCTCCTGCTGCTCTGGCTCTCAGGTGCCAGATGTgaggttca |
| | | | gctggtggagtctggTggtggcctGgtgcagccagggggctcactcAGAttgtcctgt |
| | | | gcagcttctggcttcaacattaaagacacctatatacactgggtgAGAcaggccccTg |
| | | | gAaagggcctggaatgggttgcaaggatttatcctacTaatggttatactagatatgc |
| | | | AgatagTgtcaagggcAGAttcactataagTgcagacacatccaaaaacacagcctac |
| | | | ctgcagatgaacagcctgAGAgctgaggacactgcAgtctattattgttctagatggg |
| | | | gaggggaTggcttctatgctatggacTACtggggtcaaggaaccctggtcacAgtctc |
| | | | ctcTggtggtggtggatcTggaggaggaggaAGTggaggtggaggtTCTgatatccag |
| | | | atgacccagtccccAagctccctgtcTgcctctgtgggGgatagggtcaccatcacct |
| | | | gcAGAgccagtcaggatgtgaatactgctgtagcctggtatcaacagaaaccaggaaa |
| | | | agctccCaaactactgatttactcTgcatccttcctcTACtctggagtcccttctAGA |
| | | | ttctctggatccagatctggCacTgaCttcactctgaccatcagTagtctgcagccTg |
| | | | aagacttTgcaacttattactgtcaAcaacattatactactcctcccacCttTggaca |
| | | | gggtaccaaggtggagatcaaaGGCTCTgatatTGTACTGacccagtccccTGACtcc |
| | | | ctgGCTGTGtctCTGggGGAAaggGCAaccatcAACtgcAGGAGTagtcagACCCTTC |
| | | | TGTACTCTAATAACCAGAAAAACTACCTGgcctggtatcaaAAAaaaccaggaCAGCC |
| | | | TccTaaactactgattAGTTGGgcaTTCACTAGGAAGtctggagtccctGACAGGttc |
| | | | tctggatcTGGGtcTggAacAgatttcacActgaccatcagcagtctgcagGCTgaag |
| | | | aTGTTgcaGTCtaCtactgtcagcaaTACTCAAACTACcctTGGacTttTggacaggg |
| | | | GaccaaAgtggagatcaaaAGGactgtCgctgcaccatctgtcttcatcttcccAcca |
| | | | tctgatgagcagttgaaatctggaactgcctctgttAAGtgcctgctgaataacttct |
| | | | atcccagagaggccaaagtacagtggaaggtggataaTgccctccaaAGTggtaactc |
| | | | ccaggagagtgtcacagagcaggacagcaaggacagcacctacagcctcagcagcacc |
| | | | ctgacCctgagcaaagcagactaTgagaaacacaaagtctaTgcctgTgaagtcaccc |
| | | | atcagggcctgagcACCccAgtcacaaagagcttcaacaggggagagtgttagtagta |
| | | | actcgaggacggggtgaactacgcctgaggatccgatctttttccctctgccaaaaat |
| | | | tatggggacatcatgaagccccttgagcatctgacttctggctaataaaggaaattta |
| | | | ttttcattgcaatagtgtgttggaattttttgtgtctctcactcggcctaggtagata |
| | | | agtagcatggcgggttaatcattaactacaaggaacccctagtgatggagttggccac |
| | | | tccctctctgcgcgctcgctcgctcactgaggccgggcgaccaaaggtcgcccgacgc |
| | | | ccgggctttgcccgggcggcctcagtgagcgagcgagcgcgcag |
| HER-75 | 3989 | 5190 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggaggggaTggcttctatgctatggacTACtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-77 | 3978 | 5343 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCGAAGTGCAGCTGGTGGAAAGCGGCGGAGGACTGGTGCAGCCTG |
| | | | GCGGATCTCTGAGACTGAGCTGTGCCGCCAGCGGCTTCAACATCAAGGACACCTACAT |
| | | | CCACTGGGTGCGCCAGGCCCCTGGCAAGGGACTGGAATGGGTGGCCAGAATCTACCCC |
| | | | ACCAACGGCTACACCAGATACGCCGACAGCGTGAAGGGCCGGTTCACCATCAGCGCCG |
| | | | ACACCAGCAAGAACACCGCCTACCTGCAGATGAACAGCCTGCGGGCCGAGGACACCGC |
| | | | CGTGTACTACTGTAGTAGATGGGGAGGCGACGGCTTCTACGCCATGGACTATTGGGGC |
| | | | CAGGGCACCCTCGTGACAGTGTCTAGTGCGTCGACCAAGGGGCCCTCGGTCTTCCCCC |
| | | | TGGCACCCTCCTCCAAGAGCACCTCTGGGGGCACAGCGGCCCTGGGCTGCCTGGTCAA |
| | | | GGACTACTTCCCCGAACCGGTGACGGTGTCGTGGAACTCAGGCGCCCTGACCAGCGGC |
| | | | GTGCACACCTTCCCGGCTGTCCTACAGTCCTCAGGACTCTACTCCCTCAGCAGCGTGG |
| | | | TGACCGTGCCCTCCAGCAGCTTGGGCACCCAGACCTACATCTGCAACGTGAATCACAA |
| | | | GCCCAGCAACACCAAGGTGGACAAGAAAGTTGAGCCCAAATCTTGTGACAAAACTCAC |
| | | | ACATGCCCACCGTGCCCAGCACCTGAACTCCTGGGGGGACCGTCAGTCTTCCTCTTCC |
| | | | CCCCAAAACCCAAGGACACCCTCATGATCTCCCGGACCCCTGAGGTCACATGCGTGGT |
| | | | GGTGGACGTGAGCCACGAAGACCCTGAGGTCAAGTTCAACTGGTACGTGGACGGCGTG |
| | | | GAGGTGCATAATGCCAAGACAAAGCCGCGGGAGGAGCAGTACAACAGCACGTACCGTG |
| | | | TGGTCAGCGTCCTCACCGTCCTGCACCAGGACTGGCTGAATGGCAAGGAGTACAAGTG |
| | | | CAAGGTCTCCAACAAAGCCCTCCCAGCCCCCATCGAGAAAACCATCTCCAAAGCCAAA |
| | | | GGGCAGCCCCGAGAACCACAGGTGTACACCCTGCCCCCATCCCGGGATGAGCTGACCA |
| | | | AGAACCAGGTCAGCCTGACCTGCCTGGTCAAAGGCTTCTATCCCAGCGACATCGCCGT |
| | | | GGAGTGGGAGAGCAATGGGCAGCCGGAGAACAACTACAAGACCACGCCTCCCGTGCTG |
| | | | GACTCCGACGGCTCCTTCTTCCTCTACAGCAAGCTCACCGTGGACAAGAGCAGGTGGC |
| | | | AGCAGGGGAACGTCTTCTCATGCTCCGTGATGCATGAGGCTCTGCACAACCACTACAC |
| | | | GCAGAAGAGCCTCTCCCTGTCTCCCGGCAAGagaaagaggcgagagggcagaggaagt |
| | | | cttctaacatgcggtgacgtggaggagaatcccggccctATGGACATGAGGGTCCCTG |
| | | | CTCAGCTCCTGGGGCTCCTGCTGCTCTGGCTCTCAGGTGCCAGATGTGATATCCAGAT |
| | | | GACCCAGAGCCCCAGCAGCCTGTCTGCCAGCGTGGGCGACAGAGTGACCATCACCTGT |
| | | | AGAGCCAGCCAGGACGTGAACACCGCCGTGGCCTGGTATCAGCAGAAGCCTGGCAAGG |
| | | | CCCCCAAGCTGCTGATCTACAGCGCCAGCTTCCTGTACAGCGGCGTGCCCAGCAGATT |
| | | | CAGCGGCAGCAGATCCGGCACCGACTTCACCCTGACCATCAGCTCCCTGCAGCCCGAG |
| | | | GACTTCGCCACCTACTACTGCCAGCAGCACTACACCACCCCCCCCACATTTGGCCAGG |
| | | | GCACCAAGGTGGAAATCAAGCGTACGGTGGCTGCACCATCTGTCTTCATCTTCCCGCC |
| | | | ATCTGATGAGCAGTTGAAATCTGGAACTGCCTCTGTTGTGTGCCTGCTGAATAACTTC |
| | | | TACCCCAGAGAAGCCAAAGTGCAGTGGAAGGTGGACAACGCCCTGCAGAGCGGAAACA |
| | | | GCCAGGAAAGCGTGACAGAGCAGGATTCCAAGGATTCCACATACAGCCTGAGCAGCAC |
| | | | ACTGACACTGTCCAAGGCCGACTACGAGAAGCACAAGGTGTACGCCTGCGAAGTGACA |
| | | | CACCAGGGACTGTCCTCCCCTGTGACAAAGAGCTTCAACAGAGGAGAATGCtagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-78 | 4739 | 5375 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgaggttcagct |
| | | | ggtggagtctggTggtggcctGgtgcagccagggggctcactcAGAttgtcctgtgca |
| | | | gcttctggcttcaacattaaagacacctatatacactgggtgAGAcaggccccTggAa |
| | | | agggcctggaatgggttgcaaggatttatcctacTaatggttatactagatatgcAga |
| | | | tagTgtcaagggcAGAttcactataagTgcagacacatccaaaaacacagcctacctg |
| | | | cagatgaacagcctgAGAgctgaggacactgcAgtctattattgttctagatggggag |
| | | | gggaTggcttctatgctatggacTACtggggtcaaggaaccctggtcacAgtctcctc |
| | | | TggtggtggtggatcTggaggaggaggaAGTggaggtggaggtTCTgatatccagatg |
| | | | acccagtccccAagctccctgtcTgcctctgtgggGgatagggtcaccatcacctgcA |
| | | | GAgccagtcaggatgtgaatactgctgtagcctggtatcaacagaaaccaggaaaagc |
| | | | tccCaaactactgatttactcTgcatccttcctcTACtctggagtcccttctAGAttc |
| | | | tctggatccagatctggCacTgaCttcactctgaccatcagTagtctgcagccTgaag |
| | | | acttTgcaacttattactgtcaAcaacattatactactcctcccacCttTggacaggg |
| | | | taccaaggtggagatcaaaGGCTCTgatatTGTACTGacccagtccccTGACtccctg |
| | | | GCTGTGtctCTGggGGAAaggGCAaccatcAACtgcAGGAGTagtcagACCCTTCTGT |
| | | | ACTCTAATAACCAGAAAAACTACCTGgcctggtatcaaAAAaaaccaggaCAGCCTcc |
| | | | TaaactactgattAGTTGGgcaTTCACTAGGAAGtctggagtccctGACAGGttctct |
| | | | ggatcTGGGtcTggAacAgatttcacActgaccatcagcagtctgcagGCTgaagaTG |
| | | | TTgcaGTCtaCtactgtcagcaaTACTCAAACTACcctTGGacTttTggacagggGac |
| | | | caaAgtggagatcaaaAGGactgtCgctgcaccatctgtcttcatcttcccAccatct |
| | | | gatgagcagttgaaatctggaactgcctctgttAAGtgcctgctgaataacttctatc |
| | | | ccagagaggccaaagtacagtggaaggtggataaTgccctccaaAGTggtaactccca |
| | | | ggagagtgtcacagagcaggacagcaaggacagcacctacagcctcagcagcaccctg |
| | | | acCctgagcaaagcagactaTgagaaacacaaagtctaTgcctgTgaagtcacccatc |
| | | | agggcctgagcAGCccAgtcacaaagagcttcaacaggggagagtgttagtagtaact |
| | | | cgaggacggggtgaactacgcctgaggatccgatctttttccctctgccaaaaattat |
| | | | ggggacatcatgaagccccttgagcatctgacttctggctaataaaggaaatttattt |
| | | | tcattgcaatagtgtgttggaattttttgtgtctctcactcggcctaggtagataagt |
| | | | agcatggcgggttaatcattaactacaaggaacccctagtgatggagttggccactcc |
| | | | ctctctgcgcgctcgctcgctcactgaggccgggcgaccaaaggtcgcccgacgcccg |
| | | | ggctttgcccgggcggcctcagtgagcgagcgagcgcgcag |
| HER-82 | 4505 | 5374 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCgaggttcagctggtggagtctggcggtggcctggtgcagccag |
| | | | ggggctcactccgtttgtcctgtgcagcttctggcttcaacattaaagacacctatat |
| | | | acactgggtgcgtcaggccccgggtaagggcctggaatgggttgcaaggatttatcct |
| | | | acgaatggttatactagatatgccgatagcgtcaagggccgtttcactataagcgcag |
| | | | acacatccaaaaacacagcctacctgcagatgaacagcctgcgtgctgaggacactgc |
| | | | cgtctattattgttctagatggggaggggacggcttctatgctatggacTACtggggt |
| | | | caaggaaccctggtcaccgtctcctcggcgtcgaccaagggcccatcggtcttccccc |
| | | | tggcaccctcctccaagagcacctctgggggcacagcggccctgggctgcctggtcaa |
| | | | ggactacttccccgaacctgtgacggtctcgtggaactcaggcgccctgaccagcggc |
| | | | gtgcacaccttcccggctgtcctacagtcctcaggactctactccctcagcagcgtgg |
| | | | tgaccgtgccctccagcagcttgggcacccagacctacatctgcaacgtgaatcacaa |
| | | | gcccagcaacaccaaggtggacaagaGagttgagcccaaatcttgtgacaaaactcac |
| | | | acatgcccaccgtgcccagcacctgaactcGtggggggaccgtcagtcttcctcCtcc |
| | | | ccccaaaacccaaggacaccctcatgatctcccggacccctgaggtcacatgcgtggt |
| | | | ggtggacgtgagccacgaagaccctgaggtcaagttcaactggtacgtggacggcgtg |
| | | | gaggtgcataatgccaagacaaagccgcCggaggagcagtacaacagcacgCTccgtg |
| | | | tggtcagcgtcctcaccgtcctgcaccaggactggctgaatggcaaggagtacaagtg |
| | | | caaggtctccaacaaagccctcccagcccccatcgagaaaaccatctccaaagccaaa |
| | | | gggcagccccgagaaccacaggtgtacaccctgcccccatcccgggaTgagCtgacca |
| | | | agaaccaggtcagcctgacctgcctggtcaaaggcttctatcccagcgacatcgccgt |
| | | | ggagtgggagagcaatgggcagccggagaacaactacaagaccacgcctcTcgtgctg |
| | | | gactccgacggctccttcttcctctacagcaagctcaccgtggacaagagcaggtggc |
| | | | agcaggggaacgtcttctcatgctccgtgatgcatgaggctctgcacaaccactacac |
| | | | gcagaagagcctctccctgtctccgggtaaaagaaagaggcgagagggcagaggaagt |
| | | | cttctaacatgcggtgacgtggaggagaatcccggccctATGGACATGAGGGTCCCTG |
| | | | CTCAGCTCCTGGGGCTCCTGCTGCTCTGGCTCTCAGGTGCCAGATGTGGCTCTGATCT |
| | | | GGGAAAAAAGCTTCTgGAGGCtGCcAGAGCtGGACAGGAtGAtGAAGTGagaATACTG |
| | | | ATGGCTAAtGGTGCTGATGTGAATGCCCAtGATTTTTATGGAATCACTCCCTTACACT |
| | | | TAGCTGCCAATTTtGGGCACCTgGAGATTGTGGAAGTGCTCCTGAAGCATGGaGCAGA |
| | | | TGTAAAtGCATTtGATTAtGATAACACTCCACTGCACCTGGCtGCAGATGCtGGACAT |
| | | | CTGGAGATtGTGGAAGTACTGTTAAAATAtGGAGCAGAtGTGAATGCATCtGACagaG |
| | | | ATGGACATACACCACTGCACCTGGCAGCTagaGAGGGACATCTTGAAATTGTTGAAGT |
| | | | CCTGCTTAAGAAtGGTGCtGATGTCAATGCCCAGGACAAGTTTGGGAAGACAGCTTTT |
| | | | GACATTTCAATtGATAAtGGAAAtGAGGATCTGGCtGAAATACTTCAGAAATTGggtg |
| | | | gtggtggatctggaggaggaggaAGTggaggtggaggtTCTgatatccagatgaccca |
| | | | gtccccgagctccctgtccgcctctgtgggcgatagggtcaccatcacctgccgtgcc |
| | | | agtcaggatgtgaatactgctgtagcctggtatcaacagaaaccaggaaaagctccga |
| | | | aactactgatttactcggcatccttcctcTACtctggagtcccttctcgcttctctgg |
| | | | atccagatctgggacggatttcactctgaccatcagcagtctgcagccggaagacttc |
| | | | gcaacttattactgtcagcaacattatactactcctcccacgttcggacagggtacca |
| | | | aggtggagatcaaacgaactgtggctgcaccatctgtcttcatcttcccgccatctga |
| | | | tgagcagttgaaatctggaactgcctctgttgtgtgcctgctgaataacttctatccc |
| | | | agagaggccaaagtacagtggaaggtggataacgccctccaatcgggtaactcccagg |
| | | | agagtgtcacagagcaggacagcaaggacagcacctacagcctcagcagcaccctgac |
| | | | gctgagcaaagcagactacgagaaacacaaagtctacgcctgcgaagtcacccatcag |
| | | | ggcctgagctcgcccgtcacaaagagcttcaacaggggagagtgttagtagtaactcg |
| | | | aggacggggtgaactacgcctgaggatccgatctttttccctctgccaaaaattatgg |
| | | | ggacatcatgaagccccttgagcatctgacttctggctaataaaggaaatttattttc |
| | | | attgcaatagtgtgttggaattttttgtgtctctcactcggcctaggtagataagtag |
| | | | catggcgggttaatcattaactacaaggaacccctagtgatggagttggccactccct |
| | | | ctctgcgcgctcgctcgctcactgaggccgggcgaccaaaggtcgcccgacgcccggg |
| | | | ctttgcccgggcggcctcagtgagcgagcgagcgcgcag |
| HER-88 | 3989 | 6500 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggagggTGGggcttctatgctTTTGCCCTGtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-89 | 3989 | 6501 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcgggcgggggcggggcgaggggcgggggcgggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggagggTGGggcttctatgctTTTGCCTACtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-90 | 3989 | 6502 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggagggTGGggcttctatgctTTTGACCTGtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-91 | 3989 | 6503 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggagggTGGggcttctatgctATGGCCCTGtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-92 | 3989 | 6504 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggagggGATggcttctatgctTTTGCCCTGtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-93 | 4047 | 6611 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacataacgcgtgatctaacatatcctgg |
| | | | tgtggagtagcggacgctgctatgacagaggctcgggggcctgagctggctctgtgag |
| | | | ctggggaggaggcagacagccaggccttgtctgcaagcagacctggcagcattgggct |
| | | | ggccgccccccagggcctcctcttcatgcccagtgaatgactcaccttggcacagaca |
| | | | caatgttcggggtgggcacagtgcctgcttcccgccgcaccccagcccccctcaaatg |
| | | | ccttccgagaagcccattgagcagggggcttgcattgcaccccagcctgacagcctgg |
| | | | catcttgggataaaagcagcacagccccctaggggctgcccttgctgtgtggcgccac |
| | | | cggcggtggagaacaaggctctattcagcctgtgcccaggaaaggggatcaggggatg |
| | | | cccaggcatggacagtgggtggcagggggggagaggagggctgtctgcttcccagaag |
| | | | tccaaggacacaaatgggtgaggggagagctctccccatagctgggctgcggcccaac |
| | | | cccaccccctcaggctatgccagggggtgttgccaggggcacccgggcatcgccagtc |
| | | | tagcccactccttcataaagccctcgcatcccaggagcgagcagagccagagcaggtt |
| | | | ggagaggagacgcatcacctccgctgctcgcggggatcctctagaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatactttttttgttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggaggggaTggcttctatgctatggacTACtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgc |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggggcctcagtgagcgagcgagcgcgcag |
| HER-97 | 4739 | 6505 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgaggttcagct |
| | | | ggtggagtctggTggtggcctGgtgcagccagggggctcactcAGAttgtcctgtgca |
| | | | gcttctggcttcaacattaaagacacctatatacactgggtgAGAcaggccccTggAa |
| | | | agggcctggaatgggttgcaaggatttatcctacTaatggttatactagatatgcAga |
| | | | tagTgtcaagggcAGAttcactataagTgcagacacatccaaaaacacagcctacctg |
| | | | cagatgaacagcctgAGAgctgaggacactgcAgtctattattgttctagatggggag |
| | | | ggTGGggcttctatgctTTTGCCCTGtggggtcaaggaaccctggtcacAgtctcctc |
| | | | TggtggtggtggatcTggaggaggaggaAGTggaggtggaggtTCTgatatccagatg |
| | | | acccagtccccAagctccctgtcTgcctctgtgggGgatagggtcaccatcacctgcA |
| | | | GAgccagtcaggatgtgaatactgctgtagcctggtatcaacagaaaccaggaaaagc |
| | | | tccCaaactactgatttactcTgcatccttcctcTACtctggagtcccttctAGAttc |
| | | | tctggatccagatctggCacTgaCttcactctgaccatcagTagtctgcagccTgaag |
| | | | acttTgcaacttattactgtcaAcaacattatactactcctcccacCttTggacaggg |
| | | | taccaaggtggagatcaaaGGCTCTgatatTGTACTGacccagtccccTGACtccctg |
| | | | GCTGTGtctCTGggGGAAaggGCAaccatcAACtgcAGGAGTagtcagACCCTTCTGT |
| | | | ACTCTAATAACCAGAAAAACTACCTGgcctggtatcaaAAAaaaccaggaCAGCCTcc |
| | | | TaaactactgattAGTTGGgcaTTCACTAGGAAGtctggagtccctGACAGGttctct |
| | | | ggatcTGGGtcTggAacAgatttcacActgaccatcagcagtctgcagGCTgaagaTG |
| | | | TTgcaGTCtaCtactgtcagcaaTACTCAAACTACcctTGGacTttTggacagggGac |
| | | | caaAgtggagatcaaaAGGactgtCgctgcaccatctgtcttcatcttcccAccatct |
| | | | gatgagcagttgaaatctggaactgcctctgttAAGtgcctgctgaataacttctatc |
| | | | ccagagaggccaaagtacagtggaaggtggataaTgccctccaaAGTggtaactccca |
| | | | ggagagtgtcacagagcaggacagcaaggacagcacctacagcctcagcagcaccctg |
| | | | acCctgagcaaagcagactaTgagaaacacaaagtctaTgcctgTgaagtcacccatc |
| | | | agggcctgagcAGCccAgtcacaaagagcttcaacaggggagagtgttagtagtaact |
| | | | cgaggacggggtgaactacgcctgaggatccgatctttttccctctgccaaaaattat |
| | | | ggggacatcatgaagccccttgagcatctgacttctggctaataaaggaaatttattt |
| | | | tcattgcaatagtgtgttggaattttttgtgtctctcactcggcctaggtagataagt |
| | | | agcatggcgggttaatcattaactacaaggaacccctagtgatggagttggccactcc |
| | | | ctctctgcgcgctcgctcgctcactgaggccgggcgaccaaaggtcgcccgacgcccg |
| | | | ggctttgcccgggcggcctcagtgagcgagcgagcgcgcag |
| HER-98 | 4739 | 6506 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgaggttcagct |
| | | | ggtggagtctggTggtggcctGgtgcagccagggggctcactcAGAttgtcctgtgca |
| | | | gcttctggcttcaacattaaagacacctatatacactgggtgAGAcaggccccTggAa |
| | | | agggcctggaatgggttgcaaggatttatcctacTaatggttatactagatatgcAga |
| | | | tagTgtcaagggcAGAttcactataagTgcagacacatccaaaaacacagcctacctg |
| | | | cagatgaacagcctgAGAgctgaggacactgcAgtctattattgttctagatggggag |
| | | | ggTGGggcttctatgctTTTGCCTACtggggtcaaggaaccctggtcacAgtctcctc |
| | | | TggtggtggtggatcTggaggaggaggaAGTggaggtggaggtTCTgatatccagatg |
| | | | acccagtccccAagctccctgtcTgcctctgtgggGgatagggtcaccatcacctgcA |
| | | | GAgccagtcaggatgtgaatactgctgtagcctggtatcaacagaaaccaggaaaagc |
| | | | tccCaaactactgatttactcTgcatccttcctcTACtctggagtcccttctAGAttc |
| | | | tctggatccagatctggCacTgaCttcactctgaccatcagTagtctgcagccTgaag |
| | | | acttTgcaacttattactgtcaAcaacattatactactcctcccacCttTggacaggg |
| | | | taccaaggtggagatcaaaGGCTCTgatatTGTACTGacccagtccccTGACtccctg |
| | | | GCTGTGtctCTGggGGAAaggGCAaccatcAACtgcAGGAGTagtcagACCCTTCTGT |
| | | | ACTCTAATAACCAGAAAAACTACCTGgcctggtatcaaAAAaaaccaggaCAGCCTcc |
| | | | TaaactactgattAGTTGGgcaTTCACTAGGAAGtctggagtccctGACAGGttctct |
| | | | ggatcTGGGtcTggAacAgatttcacActgaccatcagcagtctgcagGCTgaagaTG |
| | | | TTgcaGTCtaCtactgtcagcaaTACTCAAACTACcctTGGacTttTggacagggGac |
| | | | caaAgtggagatcaaaAGGactgtCgctgcaccatctgtcttcatcttcccAccatct |
| | | | gatgagcagttgaaatctggaactgcctctgttAAGtgcctgctgaataacttctatc |
| | | | ccagagaggccaaagtacagtggaaggtggataaTgccctccaaAGTggtaactccca |
| | | | ggagagtgtcacagagcaggacagcaaggacagcacctacagcctcagcagcaccctg |
| | | | acCctgagcaaagcagactaTgagaaacacaaagtctaTgcctgTgaagtcacccatc |
| | | | agggcctgagcAGCccAgtcacaaagagcttcaacaggggagagtgttagtagtaact |
| | | | cgaggacggggtgaactacgcctgaggatccgatctttttccctctgccaaaaattat |
| | | | ggggacatcatgaagccccttgagcatctgacttctggctaataaaggaaatttattt |
| | | | tcattgcaatagtgtgttggaattttttgtgtctctcactcggcctaggtagataagt |
| | | | agcatggcgggttaatcattaactacaaggaacccctagtgatggagttggccactcc |
| | | | ctctctgcgcgctcgctcgctcactgaggccgggcgaccaaaggtcgcccgacgcccg |
| | | | ggctttgcccgggcggcctcagtgagcgagcgagcgcgcag |
| HER-99 | 4739 | 6507 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgaggttcagct |
| | | | ggtggagtctggTggtggcctGgtgcagccagggggctcactcAGAttgtcctgtgca |
| | | | gcttctggcttcaacattaaagacacctatatacactgggtgAGAcaggccccTggAa |
| | | | agggcctggaatgggttgcaaggatttatcctacTaatggttatactagatatgcAga |
| | | | tagTgtcaagggcAGAttcactataagTgcagacacatccaaaaacacagcctacctg |
| | | | cagatgaacagcctgAGAgctgaggacactgcAgtctattattgttctagatggggag |
| | | | ggTGGggcttctatgctTTTgacCTGtggggtcaaggaaccctggtcacAgtctcctc |
| | | | TggtggtggtggatcTggaggaggaggaAGTggaggtggaggtTCTgatatccagatg |
| | | | acccagtccccAagctccctgtcTgcctctgtgggGgatagggtcaccatcacctgcA |
| | | | GAgccagtcaggatgtgaatactgctgtagcctggtatcaacagaaaccaggaaaagc |
| | | | tccCaaactactgatttactcTgcatccttcctcTACtctggagtcccttctAGAttc |
| | | | tctggatccagatctggCacTgaCttcactctgaccatcagTagtctgcagccTgaag |
| | | | acttTgcaacttattactgtcaAcaacattatactactcctcccacCttTggacaggg |
| | | | taccaaggtggagatcaaaGGCTCTgatatTGTACTGacccagtccccTGACtccctg |
| | | | GCTGTGtctCTGggGGAAaggGCAaccatcAACtgcAGGAGTagtcagACCCTTCTGT |
| | | | ACTCTAATAACCAGAAAAACTACCTGgcctggtatcaaAAAaaaccaggaCAGCCTcc |
| | | | TaaactactgattAGTTGGgcaTTCACTAGGAAGtctggagtccctGACAGGttctct |
| | | | ggatcTGGGtcTggAacAgatttcacActgaccatcagcagtctgcagGCTgaagaTG |
| | | | TTgcaGTCtaCtactgtcagcaaTACTCAAACTACcctTGGacTttTggacagggGac |
| | | | caaAgtggagatcaaaAGGactgtCgctgcaccatctgtcttcatcttcccAccatct |
| | | | gatgagcagttgaaatctggaactgcctctgttAAGtgcctgctgaataacttctatc |
| | | | ccagagaggccaaagtacagtggaaggtggataaTgccctccaaAGTggtaactccca |
| | | | ggagagtgtcacagagcaggacagcaaggacagcacctacagcctcagcagcaccctg |
| | | | acCctgagcaaagcagactaTgagaaacacaaagtctaTgcctgTgaagtcacccatc |
| | | | agggcctgagcAGCccAgtcacaaagagcttcaacaggggagagtgttagtagtaact |
| | | | cgaggacggggtgaactacgcctgaggatccgatctttttccctctgccaaaaattat |
| | | | ggggacatcatgaagccccttgagcatctgacttctggctaataaaggaaatttattt |
| | | | tcattgcaatagtgtgttggaattttttgtgtctctcactcggcctaggtagataagt |
| | | | agcatggcgggttaatcattaactacaaggaacccctagtgatggagttggccactcc |
| | | | ctctctgcgcgctcgctcgctcactgaggccgggcgaccaaaggtcgcccgacgcccg |
| | | | ggctttgcccgggcggcctcagtgagcgagcgagcgcgcag |
| HER-100 | 4739 | 6508 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgaggttcagct |
| | | | ggtggagtctggTggtggcctGgtgcagccagggggctcactcAGAttgtcctgtgca |
| | | | gcttctggcttcaacattaaagacacctatatacactgggtgAGAcaggccccTggAa |
| | | | agggcctggaatgggttgcaaggatttatcctacTaatggttatactagatatgcAga |
| | | | tagTgtcaagggcAGAttcactataagTgcagacacatccaaaaacacagcctacctg |
| | | | cagatgaacagcctgAGAgctgaggacactgcAgtctattattgttctagatggggag |
| | | | ggTGGggcttctatgctATGGCCCTGtggggtcaaggaaccctggtcacAgtctcctc |
| | | | TggtggtggtggatcTggaggaggaggaAGTggaggtggaggtTCTgatatccagatg |
| | | | acccagtccccAagctccctgtcTgcctctgtgggGgatagggtcaccatcacctgcA |
| | | | GAgccagtcaggatgtgaatactgctgtagcctggtatcaacagaaaccaggaaaagc |
| | | | tccCaaactactgatttactcTgcatccttcctcTACtctggagtcccttctAGAttc |
| | | | tctggatccagatctggCacTgaCttcactctgaccatcagTagtctgcagccTgaag |
| | | | acttTgcaacttattactgtcaAcaacattatactactcctcccacCttTggacaggg |
| | | | taccaaggtggagatcaaaGGCTCTgatatTGTACTGacccagtccccTGACtccctg |
| | | | GCTGTGtctCTGggGGAAaggGCAaccatcAACtgcAGGAGTagtcagACCCTTCTGT |
| | | | ACTCTAATAACCAGAAAAACTACCTGgcctggtatcaaAAAaaaccaggaCAGCCTcc |
| | | | TaaactactgattAGTTGGgcaTTCACTAGGAAGtctggagtccctGACAGGttctct |
| | | | ggatcTGGGtcTggAacAgatttcacActgaccatcagcagtctgcagGCTgaagaTG |
| | | | TTgcaGTCtaCtactgtcagcaaTACTCAAACTACcctTGGacTttTggacagggGac |
| | | | caaAgtggagatcaaaAGGactgtCgctgcaccatctgtcttcatcttcccAccatct |
| | | | gatgagcagttgaaatctggaactgcctctgttAAGtgcctgctgaataacttctatc |
| | | | ccagagaggccaaagtacagtggaaggtggataaTgccctccaaAGTggtaactccca |
| | | | ggagagtgtcacagagcaggacagcaaggacagcacctacagcctcagcagcaccctg |
| | | | acCctgagcaaagcagactaTgagaaacacaaagtctaTgcctgTgaagtcacccatc |
| | | | agggcctgagcAGCccAgtcacaaagagcttcaacaggggagagtgttagtagtaact |
| | | | cgaggacggggtgaactacgcctgaggatccgatctttttccctctgccaaaaattat |
| | | | ggggacatcatgaagccccttgagcatctgacttctggctaataaaggaaatttattt |
| | | | tcattgcaatagtgtgttggaattttttgtgtctctcactcggcctaggtagataagt |
| | | | agcatggcgggttaatcattaactacaaggaacccctagtgatggagttggccactcc |
| | | | ctctctgcgcgctcgctcgctcactgaggccgggcgaccaaaggtcgcccgacgcccg |
| | | | ggctttgcccgggcggcctcagtgagcgagcgagcgcgcag |
| HER-101 | 4739 | 6509 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCCCAAgttcagctggtgCAGtctggTGCAGAAGTTAAGAAGccag |
| | | | ggGCAtcaGTAAAGGTGTCCtgCAAGgcttctggcTACTCTTTCACTGGGTATTTCat |
| | | | aAATtgggtgAGAGAGgccccCGGCCAGggcctggaatggATGGGCCATattTCCTCC |
| | | | TCTTATGCAACTTCTACAtatAACCAGAAGTTTCAGggcAGAGTCactTTCACTGTGg |
| | | | acacatccAGCTCTacagcctacATGGAGCTGTCAagcctgAGATCTgaggacactgc |
| | | | agtctattattgtGTGagaAGTggaAACTAtGAAGAGTATgctatggacTACtggggt |
| | | | caaggaaccctggtcacAgtctcctcAgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcaggtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgaggttcagct |
| | | | ggtggagtctggTggtggcctGgtgcagccagggggctcactcAGAttgtcctgtgca |
| | | | gcttctggcttcaacattaaagacacctatatacactgggtgAGAcaggccccTggAa |
| | | | agggcctggaatgggttgcaaggatttatcctacTaatggttatactagatatgcAga |
| | | | tagTgtcaagggcAGAttcactataagTgcagacacatccaaaaacacagcctacctg |
| | | | cagatgaacagcctgAGAgctgaggacactgcAgtctattattgttctagatggggag |
| | | | ggGATggcttctatgctTTTGCCCTGtggggtcaaggaaccctggtcacAgtctcctc |
| | | | TggtggtggtggatcTggaggaggaggaAGTggaggtggaggtTCTgatatccagatg |
| | | | acccagtccccAagctccctgtcTgcctctgtgggGgatagggtcaccatcacctgcA |
| | | | GAgccagtcaggatgtgaatactgctgtagcctggtatcaacagaaaccaggaaaagc |
| | | | tccCaaactactgatttactcTgcatccttcctcTACtctggagtcccttctAGAttc |
| | | | tctggatccagatctggCacTgaCttcactctgaccatcagTagtctgcagccTgaag |
| | | | acttTgcaacttattactgtcaAcaacattatactactcctcccacCttTggacaggg |
| | | | taccaaggtggagatcaaaGGCTCTgatatTGTACTGacccagtccccTGACtccctg |
| | | | GCTGTGtctCTGggGGAAaggGCAaccatcAACtgcAGGAGTagtcagACCCTTCTGT |
| | | | ACTCTAATAACCAGAAAAACTACCTGgcctggtatcaaAAAaaaccaggaCAGCCTcc |
| | | | TaaactactgattAGTTGGgcaTTCACTAGGAAGtctggagtccctGACAGGttctct |
| | | | ggatcTGGGtcTggAacAgatttcacActgaccatcagcagtctgcagGCTgaagaTG |
| | | | TTgcaGTCtaCtactgtcagcaaTACTCAAACTACcctTGGacTttTggacagggGac |
| | | | caaAgtggagatcaaaAGGactgtCgctgcaccatctgtcttcatcttcccAccatct |
| | | | gatgagcagttgaaatctggaactgcctctgttAAGtgcctgctgaataacttctatc |
| | | | ccagagaggccaaagtacagtggaaggtggataaTgccctccaaAGTggtaactccca |
| | | | ggagagtgtcacagagcaggacagcaaggacagcacctacagcctcagcagcaccctg |
| | | | acCctgagcaaagcagactaTgagaaacacaaagtctaTgcctgTgaagtcacccatc |
| | | | agggcctgagcAGCccAgtcacaaagagcttcaacaggggagagtgttagtagtaact |
| | | | cgaggacggggtgaactacgcctgaggatccgatctttttccctctgccaaaaattat |
| | | | ggggacatcatgaagccccttgagcatctgacttctggctaataaaggaaatttattt |
| | | | tcattgcaatagtgtgttggaattttttgtgtctctcactcggcctaggtagataagt |
| | | | agcatggcgggttaatcattaactacaaggaacccctagtgatggagttggccactcc |
| | | | ctctctgcgcgctcgctcgctcactgaggccgggcgaccaaaggtcgcccgacgcccg |
| | | | ggctttgcccgggcggcctcagtgagcgagcgagcgcgcag |
| HER-102 | 3989 | 6550 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacattgattattgactagttattaatag |
| | | | taatcaattacggggtcattagttcatagcccatatatggagttccgcgttacataac |
| | | | ttacggtaaatggcccgcctggctgaccgcccaacgacccccgcccattgacgtcaat |
| | | | aatgacgtatgttcccatagtaacgccaatagggactttccattgacgtcaatgggtg |
| | | | gagtatttacggtaaactgcccacttggcagtacatcaagtgtatcatatgccaagta |
| | | | cgccccctattgacgtcaatgacggtaaatggcccgcctggcattatgcccagtacat |
| | | | gaccttatgggactttcctacttggcagtacatctacgtattagtcatcgctattacc |
| | | | atgtcgaggccacgttctgcttcactctccccatctcccccccctccccacccccaat |
| | | | tttgtatttatttattttttaattattttgtgcagcgatgggggcggggggggggggc |
| | | | gcgcgccaggcggggcggggcggggcgaggggcggggcggggcgaggcggagaggtgc |
| | | | ggcggcagccaatcagagcggcgcgctccgaaagtttccttttatggcgaggcggcgg |
| | | | cggcggcggccctataaaaagcgaagcgcgcggcgggcgggagcaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggaggggaTggcttctatgctatggacTACtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgatctccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgcaccaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcagAtggc |
| | | | agcaggggaaTgtcttctcatgcTCTgtgatgcatgaggctctgcacaaccactacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgT |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |
| HER-103 | 4047 | 7588 | ctgcgcgctcgctcgctcactgaggccgcccgggcaaagcccgggcgtcgggcgacct |
| | | | ttggtcgcccggcctcagtgagcgagcgagcgcgcagagagggagtggccaactccat |
| | | | cactaggggttccttgtagttaatgattaacccgccatgctacttatctaccagggta |
| | | | atggggatcctctagaactatagctagtcgacataacgcgtgatctaacatatcctgg |
| | | | tgtggagtagcggacgctgctatgacagaggctcgggggcctgagctggctctgtgag |
| | | | ctggggaggaggcagacagccaggccttgtctgcaagcagacctggcagcattgggct |
| | | | ggccgccccccagggcctcctcttcatgcccagtgaatgactcaccttggcacagaca |
| | | | caatgttcggggtgggcacagtgcctgcttcccgccgcaccccagcccccctcaaatg |
| | | | ccttccgagaagcccattgagcagggggcttgcattgcaccccagcctgacagcctgg |
| | | | catcttgggataaaagcagcacagccccctaggggctgcccttgctgtgtggcgccac |
| | | | cggcggtggagaacaaggctctattcagcctgtgcccaggaaaggggatcaggggatg |
| | | | cccaggcatggacagtgggtggcagggggggagaggagggctgtctgcttcccagaag |
| | | | tccaaggacacaaatgggtgaggggagagctctccccatagctgggctgcggcccaac |
| | | | cccaccccctcaggctatgccagggggtgttgccaggggcacccgggcatcgccagtc |
| | | | tagcccactccttcataaagccctcgcatcccaggagcgagcagagccagagcaggtt |
| | | | ggagaggagacgcatcacctccgctgctcgcggggatcctctagaagcttcgtttagt |
| | | | gaaccgtcagatcgcctggagacgccatccacgctgttttgacctccatagaagacac |
| | | | cgggaccgatccagcctccgcggattcgaatcccggccgggaacggtgcattggaacg |
| | | | cggattccccgtgccaagagtgacgtaagtaccgcctatagagtctataggcccacaa |
| | | | aaaatgctttcttcttttaatatacttttttgtttatcttatttctaatactttccct |
| | | | aatctctttctttcagggcaataatgatacaatgtatcatgcctctttgcaccattct |
| | | | aaagaataacagtgataatttctgggttaaggcaatagcaatatttctgcatataaat |
| | | | atttctgcatataaattgtaactgatgtaagaggtttcatattgctaatagcagctac |
| | | | aatccagctaccattctgcttttattttatggttgggataaggctggattattctgag |
| | | | tccaagctaggcccttttgctaatcatgttcatacctcttatcttcctcccacagctc |
| | | | ctgggcaacgtgctggtctgtgtgctggcccatcactttggcaaagaattgggattcg |
| | | | aaccggtgccgccaccATGGACTGGACCTGGAGGTTCCTCTTTGTGGTGGCAGCAGCT |
| | | | ACAGGTGTCCAGTCTgaggttcagctggtggagtctggAggtggcctggtgcagccag |
| | | | ggggctcactcAGAttgtcctgtgcagcttctggcttcaacattaaagacacctatat |
| | | | acactgggtgagacaggccccAggtaagggcctggaatgggttgcaaggatttatcct |
| | | | acCaatggttatactagatatgcTgatTCTgtcaagggcAGAttcactataTCTgcag |
| | | | acacatccaaaaacacagcctacctgcagatgaacagcctgAGAgctgaggacactgc |
| | | | TgtctattattgttctagatggggaggggaTggcttctatgctatggacTACtggggt |
| | | | caaggaaccctggtcacAgtctcctcTgcCtcTaccaagggcccatcTgtcttccccc |
| | | | tggcaccctcctccaagagcacctctgggggcacagcTgccctgggctgcctggtcaa |
| | | | ggactacttcccTgaacctgtgacAgtctcCtggaactcaggAgccctgaccagTggA |
| | | | gtgcacaccttcccTgctgtcctacagtcctcaggactctactccctcagcTCTgtgg |
| | | | tgacAgtgccctccagcagcttgggcacccagacctacatctgcaaTgtgaatcacaa |
| | | | gcccagcaacaccaaggtggacaagaGagttgagcccaaatcttgtgacaaaactcac |
| | | | acatgcccaccTtgcccagcacctgaactGGtggggggaccAtcagtcttcctcCtcc |
| | | | ccccaaaacccaaggacaccctcatgGCAtccAGAacccctgaggtcacatgTgtggt |
| | | | ggtggaTgtgagccaTgaagaccctgaggtcaagttcaactggtaTgtggaTggAgtg |
| | | | gaggtgcataatgccaagacaaagccCcCTgaggagcagtacaacagcacTCTcAGAg |
| | | | tggtcTCTgtcctcacAgtcctgGCCcaggactggctgaatggcaaggagtacaagtg |
| | | | caaggtctccaacaaagccctcccagcccccatTgagaaaaccatctccaaagccaaa |
| | | | gggcagcccAGAgaaccacaggtgtacaccctgcccccatccAGAgaTgagCtgacca |
| | | | agaaccaggtcagcctgacctgcctggtcaaaggcttctatcccTCTgacatTgcTgt |
| | | | ggagtgggagagcaatgggcagccTgagaacaactacaagaccacAcctcTTgtgctg |
| | | | gactcTgaTggctccttcttcctctacagcaagctcacAgtggacaagagcagAtggc |
| | | | agcaggggaaTgtcttctcatgcTCTgtgatgcatgaggctctgcacaacGCTtacac |
| | | | CcagaagagcctctccctgtctccAggtaaaagaaagaggAGAgagggcagaggaagt |
| | | | cttctaacatgTggtgaTgtggaggagaatccTggccctATGGACATGAGGGTCCCTG |
| | | | CTCAGCTCCTGGGGCTCCTGCTGCTCTGGCTCTCAGGTGCCAGATGTgatatccagat |
| | | | gacccagtccccTagctccctgtcTgcctctgtgggAgatagggtcaccatcacctgT |
| | | | AGAgccagtcaggatgtgaatactgctgtagcctggtatcaacagaaaccaggaaaag |
| | | | ctccTaaactactgatttactcTgcatccttcctcTACtctggagtcccttctAGGtt |
| | | | ctctggatccagatctgggacTgatttcactctgaccatcagcagtctgcagccAgaa |
| | | | gacttTgcaacttattactgtcagcaacattatactactcctcccacTttTggacagg |
| | | | gtaccaaggtggagatcaaaAGAactgtggctgcaccatctgtcttcatcttcccAcc |
| | | | atctgatgagcagttgaaatctggaactgcctctgttgtgtgcctgctgaataacttc |
| | | | tatcccagagaggccaaagtacagtggaaggtggataaTgccctccaatcAggtaact |
| | | | cccaggagagtgtcacagagcaggacagcaaggacagcacctacagcctcagcagcac |
| | | | cctgacTctgagcaaagcagactaTgagaaacacaaagtctaTgcctgTgaagtcacc |
| | | | catcagggcctgagctcTccTgtcacaaagagcttcaacaggggagagtgttagtagt |
| | | | aactcgaggacggggtgaactacgcctgaggatccgatctttttccctctgccaaaaa |
| | | | ttatggggacatcatgaagccccttgagcatctgacttctggctaataaaggaaattt |
| | | | attttcattgcaatagtgtgttggaattttttgtgtctctcactcggcctaggtagat |
| | | | aagtagcatggcgggttaatcattaactacaaggaacccctagtgatggagttggcca |
| | | | ctccctctctgcgcgctcgctcgctcactgaggccgggcgaccaaaggtcgcccgacg |
| | | | cccgggctttgcccgggcggcctcagtgagcgagcgagcgcgcag |

In some embodiments, the genetic element of an AAV particle described herein comprises a nucleotide sequence comprising the all of the components or a combination of the components as described in Tables 17-19, 23-26, 40, 42, 43, 68, and 69, or a sequence having at least 70%, 75%, 80%, 85%, 90%, 95%, or 99% sequence identity to the aforesaid sequences.

TABLE 68
Sequence Regions in ITR to ITR Sequences

| | HER-04 (SEQ ID NO: 5163) | | | HER-05 (SEQ ID NO: 5170) | | |
|---|---|---|---|---|---|---|
| Sequence | Region SEQ | Region | Position in SEQ ID | Region SEQ | Region | Position in SEQ |
| Regions | ID NO | length (nt) | NO: 5163 | ID NO | length (nt) | ID NO: 5170 |
| 5′ ITR | 6559 | 130 | 1-130 | 6559 | 130 | 1-130 |
| CB | 6566 | 260 | 590-849 | 6566 | 260 | 590-849 |
| promoter | | | | | | |
| Exon | 6573 | 134 | 877-1010 | 6573 | 134 | 877-1010 |
| Intron | 6578 | 32 | 1011-1042 | 6578 | 32 | 1011-1042 |
| Intron | 6580 | 347 | 1043-1389 | 6580 | 347 | 1043-1389 |
| Exon | 6576 | 53 | 1390-1442 | 6576 | 53 | 1390-1442 |
| Signal | 5157 | 57 | 1467-1523 | 5157 | 57 | 1467-1523 |
| VH | 5002 | 360 | 1524-1883 | 5171 | 357 | 1524-1880 |
| Constant | 5003 | 993 | 1884-2876 | 5011 | 990 | 1881-2870 |
| Linker | 1724 | 12 | 2877-2888 | 1724 | 12 | 2871-2882 |
| Linker | 1726 | 54 | 2889-2942 | 1726 | 54 | 2883-2936 |
| Signal | 5159 | 66 | 2943-3008 | 5159 | 66 | 2937-3002 |
| VL | 5005 | 321 | 3009-3329 | 5175 | 321 | 3003-3323 |
| Constant | 5007 | 321 | 3330-3650 | 5007 | 321 | 3324-3644 |
| PolyA | 6590 | 127 | 3693-3819 | 6590 | 127 | 3687-3813 |
| 3′ ITR | 6561 | 130 | 3863-3992 | 6561 | 130 | 3857-3986 |

In some embodiments, the AAV particle comprises a genetic element comprising the nucleotide sequence of SEQ ID NO: 5163 (HER-04), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5163, or a sequence substantially identical (e.g., having at least 70%, 75%, 80%, 85%, 90%, 95%, or 99% sequence identity) thereto. In some embodiments, the genetic element comprising the nucleotide sequence of SEQ ID NO: 5163, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566, further comprised an ie exon 1 region (SEQ ID NO: 6573), an ie intron 1 region (SEQ ID NO: 6578), a second human beta-globin intron region (SEQ ID NO: 6580), and a human beta-globin exon region (SEQ ID NO: 6576); a heavy chain signal sequence comprising the nucleotide sequence of SEQ ID NO: 5157, a heavy chain variable region comprising the nucleotide sequence of SEQ ID NO: 5002 and a heavy chain constant region comprising the nucleotide sequence of SEQ ID NO: 5003; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5005 and a light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5163 or a sequence substantially identical (e.g., having at least 70%, 75%, 80%, 85%, 90%, 95%, or 99% sequence identity) thereto.

In some embodiments, the genetic element comprising the nucleotide sequence of SEQ ID NO: 5163, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5036, a HC CDR2 amino acid sequence of SEQ ID NO: 5037, and an HC CDR3 amino acid sequence of SEQ ID NO: 5038; and a light chain variable region comprising an LC CDR1 amino acid sequence of SEQ ID NO: 5043, an LC CDR2 amino acid sequence of SEQ ID NO: 5044, and an LC CDR3 amino acid sequence of SEQ ID NO: 5045. In some embodiments, the genetic element comprising the nucleotide sequence of SEQ ID NO: 5163 or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5040, a HC CDR2 amino acid sequence of SEQ ID NO: 5041, and an HC CDR3 amino acid sequence of SEQ ID NO: 5042; and a light chain variable region comprising an LC CDR1 amino acid sequence of SEQ ID NO: 5046, an LC CDR2 amino acid sequence of SEQ ID NO: 5047, and an LC CDR3 amino acid sequence of SEQ ID NO: 5048.

In some embodiments, a genetic element comprising the sequence of SEQ ID NO: 5163, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5002 and/or a light chain variable region comprising the amino acid sequence of SEQ ID NO: 5006; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5163, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5034 and/or a light chain comprising the amino acid sequence of SEQ ID NO: 5036, or a sequence substantially identical (e.g., having at least 70%, 75%, 80%, 85%, 90%, 95%, or 99% sequence identity) to the aforesaid sequences.

In some embodiments, the AAV particle comprises a genetic element comprising the nucleotide sequence of SEQ ID NO: 5170 (HER-05), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5170, or a sequence substantially identical (e.g., having at least 70%, 75%, 80%, 85%, 90%, 95%, or 99% sequence identity) thereto. In some embodiments, the genetic element comprising the nucleotide sequence of SEQ ID NO: 5170, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566, further comprised an ie exon 1 region (SEQ ID NO: 6573), an ie intron 1 region (SEQ ID NO: 6578), a second human beta-globin intron region (SEQ ID NO: 6580), and a human beta-globin exon region (SEQ ID NO: 6576); a heavy chain signal sequence comprising the nucleotide sequence of SEQ ID NO: 5157, a heavy chain variable region comprising the nucleotide sequence of SEQ ID NO: 5171 and a heavy chain constant region comprising the nucleotide sequence of SEQ ID NO: 5011; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5175 and a light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5170 or a sequence substantially identical (e.g., having at least 70%, 75%, 80%, 85%, 90%, 95%, or 99% sequence identity) thereto.

In some embodiments, the genetic element comprising the nucleotide sequence of SEQ ID NO: 5170, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5179, a HC CDR2 amino acid sequence of SEQ ID NO: 5180, and an HC CDR3 amino acid sequence of SEQ ID NO: 5181; and a light chain variable region comprising an LC CDR1 amino acid sequence of SEQ ID NO: 5182, an LC CDR2 amino acid sequence of SEQ ID NO: 5183, and an LC CDR3 amino acid sequence of SEQ ID NO: 5184.

In some embodiments, a genetic element comprising the sequence of SEQ ID NO: 5170, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5172 and/or a light chain variable region comprising the amino acid sequence of SEQ ID NO: 5176; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5170, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5174 and/or a light chain comprising the amino acid sequence of SEQ ID NO: 5187, or a sequence substantially identical (e.g., having at least 70%, 75%, 80%, 85%, 90%, 95%, or 99% sequence identity) to the aforesaid sequences.

TABLE 69
Sequence Regions in ITR to ITR Sequences

HER-15 (SEQ ID NO: 5165)

| Sequence | Region SEQ | Region | Position in SEQ |
|---|---|---|---|
| Regions | ID NO | length (nt) | ID NO: 5165 |
| 5′ ITR | 6559 | 130 | 1-130 |
| CMV(IE) | 6594 | 380 | 204-583 |
| CB promoter | 6566 | 260 | 590-849 |
| Intron | 6595 | 566 | 877-1442 |
| Signal (H5) | 5157 | 57 | 1467-1523 |
| VH | 5009 | 357 | 1524-1880 |
| Constant | 5011 | 990 | 1881-2870 |
| Linker | 1724 | 12 | 2871-2882 |
| Linker | 1726 | 54 | 2883-2936 |
| Signal | 5159 | 66 | 2937-3002 |
| C12 Fynomer | 5155 | 189 | 3003-3191 |
| Linker | 5347 | 45 | 3192-3236 |
| VL | 5013 | 321 | 3237-3557 |
| Constant | 5007 | 321 | 3558-3878 |
| PolyA | 6590 | 127 | 3921-4047 |
| 3′ ITR | 6561 | 130 | 4091-4220 |

In some embodiments, the AAV particle comprises a genetic element comprising the nucleotide sequence of SEQ ID NO: 5165 (HER-15), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5165, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5165, comprises in 5′ to 3′ order: a 5′ inverted terminal repeat (ITR) sequence region comprising the nucleotide sequence of SEQ ID NO: 6559; a CMV(IE) promoter region comprising the nucleotide sequence of SEQ ID NO: 6594 and a CB promoter comprising the nucleotide sequence of SEQ ID NO: 6566; an intron region comprising the nucleotide sequence of SEQ ID NO: 6595; a heavy chain signal sequence comprising the nucleotide sequence of SEQ ID NO: 5157, a heavy chain variable region comprising the nucleotide sequence of SEQ ID NO: 5009, and heavy chain constant region comprising the nucleotide sequence of SEQ ID NO: 5011; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159; a Fynomer comprising the nucleotide sequence of SEQ ID NO: 5155; a linker comprising the nucleotide sequence of SEQ ID NO: 5347; a light chain variable region comprising the nucleotide sequence of SEQ ID NO: 5013 and light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5165 or a sequence substantially identical (e.g., having at least 70%, 75%, 80%, 85%, 90%, 95%, or 99% sequence identity) thereto.

In some embodiments, the genetic element comprising the nucleotide sequence of SEQ ID NO: 5165, or a sequence substantially identical (e.g., having at least 70%, 75%, 80%, 85%, 90%, 95%, or 99% sequence identity) thereto, encodes a multispecific antibody molecule, e.g., bispecific or biparatopic, antibody molecule, comprising a first antigen binding domain that binds domain II of a HER2 antigen and a second antigen binding domain that binds domain I of a HER2 antigen. In some embodiments, the first antigen binding domain comprises a full antibody. In some embodiments, the second antigen binding domain comprises a C12 fynomer. In some embodiments, the first antigen binding domain comprises a heavy chain variable region comprising a HC CDR1 amino acid sequence of SEQ ID NO: 5149, a HC CDR2 amino acid sequence of SEQ ID NO: 5150, and an HC CDR3 amino acid sequence of SEQ ID NO: 5151; and a light chain variable region comprising an LC CDR1 amino acid sequence of SEQ ID NO: 5152, an LC CDR2 amino acid sequence of SEQ ID NO: 5153, and an LC CDR3 amino acid sequence of SEQ ID NO: 5154. In some embodiments, the second antigen binding domain comprises the amino acid sequence of SEQ ID NO: 5156, or an amino acid sequence substantially identical (e.g., having at least 70%, 75%, 80%, 85%, 90%, 95%, or 99% sequence identity) thereto.

In some embodiments, a genetic element comprising the sequence of SEQ ID NO: 5165, or a sequence substantially identical (e.g., having at least 70%, 75%, 80%, 85%, 90%, 95%, or 99% sequence identity) thereto, encodes a multispecific antibody molecule, comprising: a first chain, comprising from the N-terminus to the C-terminus, an anti-HER2 VH comprising the amino sequence comprising the amino acid sequence of SEQ ID NO: 5010, and a heavy chain constant region comprising the amino acid sequence of SEQ ID NO: 5012; and a second chain, comprising from the N-terminus to the C-terminus, a C12 fynomer comprising the amino acid sequence of SEQ ID NO: 5156, a (G4S)3 linker (SEQ ID NO: 4537), an anti-HER2 VL comprising the amino acid sequence of SEQ ID NO: 5014, and a light chain constant region (CL) comprising the amino acid sequence of SEQ ID NO: 5008; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5165, or a sequence substantially identical (e.g., having at least 70%, 75%, 80%, 85%, 90%, 95%, or 99% sequence identity) to the any of the aforesaid sequences, encodes a multispecific antibody molecule, comprising a first chain comprising the amino acid sequence of SEQ ID NO: 5066; and a second chain comprising the amino acid sequence of SEQ ID NO: 5156, a (G4S)3 linker (SEQ ID NO: 4537), and the amino acid sequence comprising of SEQ ID NO: 5068; or a sequence substantially identical (e.g., having at least 70%, 75%, 80%, 85%, 90%, 95%, or 99% sequence identity) to the any of the aforesaid sequences.

TABLE 17
Sequence Regions in ITR to ITR Sequences

| | HER-10 (SEQ ID NO: 5164) | | | HER-42 (SEQ ID NO: 5166) | | | HER-47 (SEQ ID NO: 5167) | | | HER-53 (SEQ ID NO: 5168) | | |
|---|---|---|---|---|---|---|---|---|---|---|---|---|
| | Region | Region | Position in | Region | Region | Position in | Region | Region | Position in | Region | Region | Position in |
| Sequence | SEQ ID | length | SEQ ID | SEQ ID | length | SEQ ID | SEQ ID | length | SEQ ID | SEQ ID | length | SEQ ID |
| Regions | NO | (nt) | NO: 5164 | NO | (nt) | NO: 5166 | NO | (nt) | NO: 5167 | NO | (nt) | NO: 5168 |
| 5′ ITR | 6559 | 130 | 1-130 | 6559 | 130 | 1-130 | 6559 | 130 | 1-130 | 6559 | 130 | 1-130 |
| CMV(IE) | 6594 | 380 | 204-583 | 6594 | 380 | 204-583 | 6594 | 380 | 204-583 | 6594 | 380 | 204-583 |
| CB | 6566 | 260 | 590-849 | 6566 | 260 | 590-849 | 6566 | 260 | 590-849 | 6566 | 260 | 590-849 |
| promoter | | | | | | | | | | | | |
| Intron | 6595 | 566 | 877-1442 | 6595 | 566 | 877-1442 | 6595 | 566 | 877-1442 | 6595 | 566 | 877-1442 |
| Signal | 5157 | 57 | 1467-1523 | 5157 | 57 | 1467-1523 | 5157 | 57 | 1467-1523 | 5157 | 57 | 1467-1523 |
| (H5) | | | | | | | | | | | | |
| VH | 5015 | 360 | 1524-1883 | 5070 | 360 | 1524-1883 | 5089 | 360 | 1524-1883 | 5109 | 360 | 1524-1883 |
| Constant | 5017 | 990 | 1884-2873 | 5025 | 993 | 1884-2876 | 5029 | 993 | 1884-2876 | 5017 | 990 | 1884-2873 |
| Linker | 1724 | 12 | 2874-2885 | 1724 | 12 | 2877-2888 | 1724 | 12 | 2877-2888 | 1724 | 12 | 2874-2885 |
| Linker | 1726 | 54 | 2886-2939 | 1726 | 54 | 2889-2942 | 1726 | 54 | 2889-2942 | 1726 | 54 | 2886-2939 |
| Signal | 5159 | 66 | 2940-3005 | 5159 | 66 | 2943-3008 | 5159 | 66 | 2943-3008 | 5159 | 66 | 2940-3005 |
| VL | 5019 | 321 | 3006-3326 | 5085 | 321 | 3009-3329 | 5093 | 321 | 3009-3329 | 5113 | 321 | 3006-3326 |
| Constant | 5021 | 322 | 3327-3648 | 5007 | 321 | 3330-3650 | 5007 | 321 | 3330-3650 | 5007 | 321 | 3327-3648 |
| PolyA | 6590 | 127 | 3690-3816 | 6590 | 127 | 3693-3819 | 6590 | 127 | 3693-3819 | 6590 | 127 | 3690-3816 |
| 3′ ITR | 6561 | 130 | 3860-3989 | 6561 | 130 | 3863-3992 | 6561 | 130 | 3863-3992 | 6561 | 130 | 3860-3989 |

In some embodiments, the AAV particle comprises a genetic element comprising the nucleotide sequence of SEQ ID NO: 5164 (HER-10), or a nucleotide sequence substantially identical (e.g., having at least 70%, 75%, 80%, 85%, 90%, 95%, 99% or 100%, sequence identity) thereto. In some embodiments, the genetic element comprises SEQ ID NO: 5164, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5164, comprises in 5′ to 3′ order: a 5′ inverted terminal repeat (ITR) sequence region comprising the nucleotide sequence of SEQ ID NO: 6559; a CMV(IE) promoter comprising the nucleotide sequence of SEQ ID NO: 6594; a CB promoter comprising the nucleotide sequence of SEQ ID NO: 6566; a human beta-globin intron region comprising the nucleotide sequence of SEQ ID NO: 6595; a heavy chain signal sequence comprising the nucleotide sequence of SEQ ID NO: 5157, a heavy chain variable region comprising the nucleotide sequence of SEQ ID NO: 5015 and heavy chain constant region comprising the nucleotide sequence of SEQ ID NO: 5017; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5019 and light chain constant region comprising the nucleotide sequence of SEQ ID NO: 5021; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5164 or a sequence substantially identical (e.g., having at least 70%, 75%, 80%, 85%, 90%, 95%, or 99% sequence identity) thereto.

In some embodiments, a genetic element comprising the sequence of SEQ ID NO: 5164, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5053, a HC CDR2 amino acid sequence of SEQ ID NO: 5054, and an HC CDR3 amino acid sequence of SEQ ID NO: 5055; and a light chain variable region comprising an LC CDR1 amino acid sequence of SEQ ID NO: 5059, an LC CDR2 amino acid sequence of SEQ ID NO: 5060, and an LC CDR3 amino acid sequence of SEQ ID NO: 5061. In some embodiments, a genetic element comprising the sequence of SEQ ID NO: 5164, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5056, a HC CDR2 amino acid sequence of SEQ ID NO: 5057, and an HC CDR3 amino acid sequence of SEQ ID NO: 5058; and a light chain variable region comprising an LC CDR1 amino acid sequence of SEQ ID NO: 5062, an LC CDR2 amino acid sequence of SEQ ID NO: 5063, and an LC CDR3 amino acid sequence of SEQ ID NO: 5064.

In some embodiments, a genetic element comprising the sequence of SEQ ID NO: 5164, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5016 and/or a light chain variable region comprising the amino acid sequence of SEQ ID NO: 5020, or a sequence substantially identical (e.g., having at least 70%, 75%, 80%, 85%, 90%, 95%, or 99% sequence identity) to the aforesaid sequences.

In some embodiments, a genetic element comprising the sequence of SEQ ID NO: 5164, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5050 and/or a light chain comprising the amino acid sequence of SEQ ID NO: 5052 or a sequence substantially identical (e.g., having at least 70%, 75%, 80%, 85%, 90%, 95%, or 99% sequence identity) to the any of the aforesaid.

In some embodiments, the AAV particle comprises a genetic element comprising the nucleotide sequence of SEQ ID NO: 5166 (HER-42), or a nucleotide sequence substantially identical (e.g., having at least 70%, 75%, 80%, 85%, 90%, 95%, 99% or 100%, sequence identity) thereto. In some embodiments, the genetic element comprises SEQ ID NO: 5166, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5166, comprises in 5′ to 3′ order: a 5′ inverted terminal repeat (ITR) sequence region comprising the nucleotide sequence of SEQ ID NO: 6559; a CMV(IE) promoter comprising the nucleotide sequence of SEQ ID NO: 6594; a CB promoter comprising the nucleotide sequence of SEQ ID NO: 6566; a human beta-globin intron region comprising the nucleotide sequence of SEQ ID NO: 6595; a heavy chain signal sequence comprising the nucleotide sequence of SEQ ID NO: 5157, a heavy chain variable region comprising the nucleotide sequence of SEQ ID NO: 5070 and heavy chain constant region comprising the nucleotide sequence of SEQ ID NO: 5025; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5085 and light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5166 or a sequence substantially identical (e.g., having at least 70%, 75%, 80%, 85%, 90%, 95%, or 99% sequence identity) thereto.

In some embodiments, a genetic element comprising the sequence of SEQ ID NO: 5166, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5071, a HC CDR2 amino acid sequence of SEQ ID NO: 5072, and an HC CDR3 amino acid sequence of SEQ ID NO: 5073; and a light chain variable region comprising an LC CDR1 amino acid sequence of SEQ ID NO: 5077, an LC CDR2 amino acid sequence of SEQ ID NO: 5078, and an LC CDR3 amino acid sequence of SEQ ID NO: 5079. In some embodiments, a genetic element comprising the sequence of SEQ ID NO: 5166, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5074, a HC CDR2 amino acid sequence of SEQ ID NO: 5075, and an HC CDR3 amino acid sequence of SEQ ID NO: 5076; and a light chain variable region comprising an LC CDR1 amino acid sequence of SEQ ID NO: 5080, an LC CDR2 amino acid sequence of SEQ ID NO: 5081, and an LC CDR3 amino acid sequence of SEQ ID NO: 5082.

In some embodiments, a genetic element comprising the sequence of SEQ ID NO: 5166, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5069 and/or a light chain variable region comprising the amino acid sequence of SEQ ID NO: 5086, or a sequence substantially identical (e.g., having at least 70%, 75%, 80%, 85%, 90%, 95%, or 99% sequence identity) to the aforesaid sequences.

In some embodiments, a genetic element comprising the sequence of SEQ ID NO: 5166, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5084 and/or a light chain comprising the amino acid sequence of SEQ ID NO: 5088 or a sequence substantially identical (e.g., having at least 70%, 75%, 80%, 85%, 90%, 95%, or 99% sequence identity) to the any of the aforesaid.

In some embodiments, the AAV particle comprises a genetic element comprising the nucleotide sequence of SEQ ID NO: 5167 (HER-47), or a nucleotide sequence substantially identical (e.g., having at least 70%, 75%, 80%, 85%, 90%, 95%, 99% or 100%, sequence identity) thereto. In some embodiments, the genetic element comprises SEQ ID NO: 5167, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5167, comprises in 5′ to 3′ order: a 5′ inverted terminal repeat (ITR) sequence region comprising the nucleotide sequence of SEQ ID NO: 6559; a CMV(IE) promoter comprising the nucleotide sequence of SEQ ID NO: 6594; a CB promoter comprising the nucleotide sequence of SEQ ID NO: 6566; a human beta-globin intron region comprising the nucleotide sequence of SEQ ID NO: 6595; a heavy chain signal sequence comprising the nucleotide sequence of SEQ ID NO: 5157, a heavy chain variable region comprising the nucleotide sequence of SEQ ID NO: 5089 and heavy chain constant region comprising the nucleotide sequence of SEQ ID NO: 5029; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5093 and light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5167 or a sequence substantially identical (e.g., having at least 70%, 75%, 80%, 85%, 90%, 95%, or 99% sequence identity) thereto.

In some embodiments, a genetic element comprising the sequence of SEQ ID NO: 5167, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5097, a HC CDR2 amino acid sequence of SEQ ID NO: 5098, and an HC CDR3 amino acid sequence of SEQ ID NO: 5099; and a light chain variable region comprising an LC CDR1 amino acid sequence of SEQ ID NO: 5103, an LC CDR2 amino acid sequence of SEQ ID NO: 5104, and an LC CDR3 amino acid sequence of SEQ ID NO: 5105. In some embodiments, a genetic element comprising the sequence of SEQ ID NO: 5167, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5100, a HC CDR2 amino acid sequence of SEQ ID NO: 5101, and an HC CDR3 amino acid sequence of SEQ ID NO: 5102; and a light chain variable region comprising an LC CDR1 amino acid sequence of SEQ ID NO: 5106, an LC CDR2 amino acid sequence of SEQ ID NO: 5107, and an LC CDR3 amino acid sequence of SEQ ID NO: 5108.

In some embodiments, a genetic element comprising the sequence of SEQ ID NO: 5167, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5090 and/or a light chain variable region comprising the amino acid sequence of SEQ ID NO: 5094, or a sequence substantially identical (e.g., having at least 70%, 75%, 80%, 85%, 90%, 95%, or 99% sequence identity) to the aforesaid sequences.

In some embodiments, a genetic element comprising the sequence of SEQ ID NO: 5167, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5092 and/or a light chain comprising the amino acid sequence of SEQ ID NO: 5096 or a sequence substantially identical (e.g., having at least 70%, 75%, 80%, 85%, 90%, 95%, or 99% sequence identity) to the any of the aforesaid.

In some embodiments, the AAV particle comprises a genetic element comprising the nucleotide sequence of SEQ ID NO: 5168 (HER-53), or a nucleotide sequence substantially identical (e.g., having at least 70%, 75%, 80%, 85%, 90%, 95%, 99% or 100%, sequence identity) thereto. In some embodiments, the genetic element comprises SEQ ID NO: 5168, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5168, comprises in 5′ to 3′ order: a 5′ inverted terminal repeat (ITR) sequence region comprising the nucleotide sequence of SEQ ID NO: 6559; a CMV(IE) promoter comprising the nucleotide sequence of SEQ ID NO: 6594; a CB promoter comprising the nucleotide sequence of SEQ ID NO: 6566; a human beta-globin intron region comprising the nucleotide sequence of SEQ ID NO: 6595; a heavy chain signal sequence comprising the nucleotide sequence of SEQ ID NO: 5157, a heavy chain variable region comprising the nucleotide sequence of SEQ ID NO: 5109 and heavy chain constant region comprising the nucleotide sequence of SEQ ID NO: 5017; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5113 and light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5168 or a sequence substantially identical (e.g., having at least 70%, 75%, 80%, 85%, 90%, 95%, or 99% sequence identity) thereto.

In some embodiments, a genetic element comprising the sequence of SEQ ID NO: 5168, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5117, a HC CDR2 amino acid sequence of SEQ ID NO: 5118, and an HC CDR3 amino acid sequence of SEQ ID NO: 5119; and a light chain variable region comprising an LC CDR1 amino acid sequence of SEQ ID NO: 5123, an LC CDR2 amino acid sequence of SEQ ID NO: 5124, and an LC CDR3 amino acid sequence of SEQ ID NO: 5125. In some embodiments, a genetic element comprising the sequence of SEQ ID NO: 5168, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5120, a HC CDR2 amino acid sequence of SEQ ID NO: 5121, and an HC CDR3 amino acid sequence of SEQ ID NO: 5122; and a light chain variable region comprising an LC CDR1 amino acid sequence of SEQ ID NO: 5126, an LC CDR2 amino acid sequence of SEQ ID NO: 5127, and an LC CDR3 amino acid sequence of SEQ ID NO: 5128.

In some embodiments, a genetic element comprising the sequence of SEQ ID NO: 5168, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5110 and/or a light chain variable region comprising the amino acid sequence of SEQ ID NO: 5114, or a sequence substantially identical (e.g., having at least 70%, 75%, 80%, 85%, 90%, 95%, or 99% sequence identity) to the aforesaid sequences.

In some embodiments, a genetic element comprising the sequence of SEQ ID NO: 5168, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5112 and/or a light chain comprising the amino acid sequence of SEQ ID NO: 5116 or a sequence substantially identical (e.g., having at least 70%, 75%, 80%, 85%, 90%, 95%, or 99% sequence identity) to the any of the aforesaid.

TABLE 18
Sequence Regions in ITR to ITR Sequences

| | HER-43 (SEQ ID NO: 5185) | | | HER-46 (SEQ ID NO: 5186) | | |
|---|---|---|---|---|---|---|
| | | Region | | | Region | |
| Sequence | Region | length | Position in SEQ | Region SEQ | length | Position in SEQ |
| Regions | SEQ ID NO | (nt) | ID NO: 5185 | ID NO | (nt) | ID NO: 5186 |
| 5′ ITR | 6559 | 130 | 1-130 | 6559 | 130 | 1-130 |
| CB | 6566 | 260 | 590-849 | 6566 | 260 | 590-849 |
| promoter | | | | | | |
| Exon | 6573 | 134 | 877-1010 | 6573 | 134 | 877-1010 |
| Intron | 6578 | 32 | 1011-1042 | 6578 | 32 | 1011-1042 |
| Intron | 6580 | 347 | 1043-1389 | 6580 | 347 | 1043-1389 |
| Exon | 6576 | 53 | 1390-1442 | 6576 | 53 | 1390-1442 |
| Signal | 5157 | 57 | 1467-1523 | 5157 | 57 | 1467-1523 |
| VH | 5191 | 360 | 1524-1883 | 5223 | 360 | 1524-1883 |
| Constant | 5211 | 993 | 1884-2876 | 5213 | 993 | 1884-2876 |
| Linker | 1724 | 12 | 2877-2888 | 1724 | 12 | 2877-2888 |
| Linker | 1726 | 54 | 2889-2942 | 1726 | 54 | 2889-2942 |
| Signal | 5159 | 66 | 2943-3008 | 5159 | 66 | 2943-3008 |
| VL | 5195 | 321 | 3009-3329 | 5227 | 321 | 3009-3329 |
| Constant | 5007 | 321 | 3330-3650 | 5007 | 321 | 3330-3650 |
| PolyA | 6590 | 127 | 3693-3819 | 6590 | 127 | 3693-3819 |
| 3′ ITR | 6561 | 130 | 3863-3992 | 6561 | 130 | 3863-3992 |

In some embodiments, the AAV particle comprises a genetic element comprising the nucleotide sequence of SEQ ID NO: 5185 (HER-43), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5185, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5185, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5191, and heavy chain constant region comprising the nucleotide sequence of SEQ ID NO: 5211; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5195 and light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5185 or a sequence substantially identical (e.g., having at least 70%, 75%, 80%, 85%, 90%, 95%, or 99% sequence identity) thereto.

In some embodiments, a genetic element comprising the sequence of SEQ ID NO: 5185, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5199, a HC CDR2 amino acid sequence of SEQ ID NO: 5200, and an HC CDR3 amino acid sequence of SEQ ID NO: 5201; and a light chain variable region comprising an LC CDR1 amino acid sequence of SEQ ID NO: 5205, an LC CDR2 amino acid sequence of SEQ ID NO: 5206, and an LC CDR3 amino acid sequence of SEQ ID NO: 5207. In some embodiments, a genetic element comprising the sequence of SEQ ID NO: 5185, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5202, a HC CDR2 amino acid sequence of SEQ ID NO: 5203, and an HC CDR3 amino acid sequence of SEQ ID NO: 5204; and a light chain variable region comprising an LC CDR1 amino acid sequence of SEQ ID NO: 5208, an LC CDR2 amino acid sequence of SEQ ID NO: 5209, and an LC CDR3 amino acid sequence of SEQ ID NO: 5210.

In some embodiments, a genetic element comprising the sequence of SEQ ID NO: 5185, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5192 and/or a light chain variable region comprising the amino acid sequence of SEQ ID NO: 5196; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5185, or a sequence substantially identical (e.g., having at least 70%, 75%, 80%, 85%, 90%, 95%, or 99% sequence identity) to the any of the aforesaid sequences, encodes an antibody molecule comprising a heavy chain comprising the amino acid sequence of SEQ ID NO: 5194 and/or a light chain comprising the amino acid sequence of SEQ ID NO: 5198; or a sequence substantially identical (e.g., having at least 70%, 75%, 80%, 85%, 90%, 95%, or 99% sequence identity) to the any of the aforesaid sequences.

In some embodiments, the AAV particle comprises a genetic element comprising the nucleotide sequence of SEQ ID NO: 5186 (HER-46), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5186, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5186, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5223, and heavy chain constant region comprising the nucleotide sequence of SEQ ID NO: 5213; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5227 and light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5186 or a sequence substantially identical (e.g., having at least 70%, 75%, 80%, 85%, 90%, 95%, or 99% sequence identity) thereto.

In some embodiments, a genetic element comprising the sequence of SEQ ID NO: 5186, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5231, a HC CDR2 amino acid sequence of SEQ ID NO: 5232, and an HC CDR3 amino acid sequence of SEQ ID NO: 5233; and a light chain variable region comprising an LC CDR1 amino acid sequence of SEQ ID NO: 5237, an LC CDR2 amino acid sequence of SEQ ID NO: 5238, and an LC CDR3 amino acid sequence of SEQ ID NO: 5239. In some embodiments, a genetic element comprising the sequence of SEQ ID NO: 5186, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5234, a HC CDR2 amino acid sequence of SEQ ID NO: 5235, and an HC CDR3 amino acid sequence of SEQ ID NO: 5236; and a light chain variable region comprising an LC CDR1 amino acid sequence of SEQ ID NO: 5240, an LC CDR2 amino acid sequence of SEQ ID NO: 5241, and an LC CDR3 amino acid sequence of SEQ ID NO: 5242.

In some embodiments, a genetic element comprising the sequence of SEQ ID NO: 5186, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5224 and/or a light chain variable region comprising the amino acid sequence of SEQ ID NO: 5228; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5186, or a sequence substantially identical (e.g., having at least 70%, 75%, 80%, 85%, 90%, 95%, or 99% sequence identity) to the any of the aforesaid sequences, encodes an antibody molecule comprising a heavy chain comprising the amino acid sequence of SEQ ID NO: 5226 and/or a light chain comprising the amino acid sequence of SEQ ID NO: 5230; or a sequence substantially identical (e.g., having at least 70%, 75%, 80%, 85%, 90%, 95%, or 99% sequence identity) to the any of the aforesaid sequences.

TABLE 19
Sequence Regions in ITR to ITR Sequences

HER-57 (SEQ ID NO: 5187)

| Sequence | Region SEQ | Region | Position in |
|---|---|---|---|
| Regions | ID NO | length (nt) | SEQ ID NO: 5187 |
| 5′ ITR | 6559 | 130 | 1-130 |
| CMV(IE) | 6594 | 380 | 204-583 |
| CB promoter | 6566 | 260 | 590-849 |
| Exon | 6573 | 134 | 877-1010 |
| Intron | 6578 | 32 | 1011-1042 |
| Intron | 6580 | 347 | 1043-1389 |
| Exon | 6576 | 53 | 1390-1442 |
| Signal (H5) | 5157 | 57 | 1467-1523 |
| VH | 5253 | 360 | 1524-1883 |
| Linker | 5347 | 45 | 1884-1928 |
| VL | 5255 | 321 | 1929-2249 |
| Constant | 5277 | 696 | 2250-2945 |
| PolyA | 6590 | 127 | 2988-3114 |
| 3′ ITR | 6561 | 130 | 3158-3287 |

In some embodiments, the AAV particle comprises a genetic element comprising the nucleotide sequence of SEQ ID NO: 5187 (HER-57), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5187, or a sequence substantially identical (e.g., having at least 70%, 75%, 80%, 85%, 90%, 95%, or 99% sequence identity) thereto. In some embodiments, the genetic element comprising the nucleotide sequence of SEQ ID NO: 5187 comprises in 5′ to 3′ order: 5′ inverted terminal repeat (ITR) sequence region comprising the nucleotide sequence of SEQ ID NO: 6559; a CMV(IE) promoter comprising the nucleotide sequence of SEQ ID NO: 6594 and a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5253, a linker region comprising the nucleotide sequence of SEQ ID NO: 5347; a light chain variable region comprising the nucleotide sequence of SEQ ID NO: 5255, and light chain constant region comprising the nucleotide sequence of SEQ ID NO:5277; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5187 or a sequence substantially identical (e.g., having at least 70%, 75%, 80%, 85%, 90%, 95%, or 99% sequence identity) thereto.

In some embodiments, a genetic element comprising the sequence of SEQ ID NO: 5187, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5293, a HC CDR2 amino acid sequence of SEQ ID NO: 5294, and an HC CDR3 amino acid sequence of SEQ ID NO: 5295; and a light chain variable region comprising an LC CDR1 amino acid sequence of SEQ ID NO: 5299, an LC CDR2 amino acid sequence of SEQ ID NO: 5300, and an LC CDR3 amino acid sequence of SEQ ID NO: 5301. In some embodiments, a genetic element comprising the sequence of SEQ ID NO: 5187, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5296, a HC CDR2 amino acid sequence of SEQ ID NO: 5297, and an HC CDR3 amino acid sequence of SEQ ID NO: 5298; and a light chain variable region comprising an LC CDR1 amino acid sequence of SEQ ID NO: 5302, an LC CDR2 amino acid sequence of SEQ ID NO: 5303, and an LC CDR3 amino acid sequence of SEQ ID NO: 5304.

In some embodiments, a genetic element comprising the sequence of SEQ ID NO: 5187, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5254 and/or a light chain variable region comprising the amino acid sequence of SEQ ID NO: 5256; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5187, or a sequence substantially identical (e.g., having at least 70%, 75%, 80%, 85%, 90%, 95%, or 99% sequence identity) thereto, encodes an antibody molecule comprising an scFv comprising the amino acid sequence of SEQ ID NO: 5351 and/or an Fc region comprising the amino acid sequence of SEQ ID NO: 5278; or a sequence substantially identical (e.g., having at least 70%, 75%, 80%, 85%, 90%, 95%, or 99% sequence identity) to the any of the aforesaid sequences.

TABLE 23
Sequence Regions in ITR to ITR Sequences

HER-69 (SEQ ID NO: 5169)

| Sequence | Region SEQ | Region | Position in |
|---|---|---|---|
| Regions | ID NO | length (nt) | SEQ ID NO: 5169 |
| 5′ ITR | 6559 | 130 | 1-130 |
| GFAP | 6568 | 699 | 216-914 |
| Promoter | | | |
| Intron | 6595 | 566 | 935-1500 |
| Signal (H5) | 5157 | 57 | 1525-1581 |
| VH | 5129 | 360 | 1582-1941 |
| Constant | 5017 | 990 | 1942-2931 |
| Linker | 1724 | 12 | 2932-2943 |
| Linker | 1726 | 54 | 2944-2997 |
| Signal | 5159 | 66 | 2998-3063 |
| VL | 5133 | 321 | 3064-3384 |
| Constant | 5021 | 322 | 3385-3706 |
| PolyA | 6590 | 127 | 3748-3874 |
| 3′ ITR | 6561 | 130 | 3918-4047 |

In some embodiments, the AAV particle comprises a genetic element comprising SEQ ID NO: 5169 (HER-69), or a nucleotide sequence substantially identical (e.g., having at least 70%, 75%, 80%, 85%, 90%, 95%, 99% or 100%, sequence identity) thereto. In some embodiments, the genetic element comprises SEQ ID NO: 5169, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5169, comprises in 5′ to 3′ order: a 5′ inverted terminal repeat (ITR) sequence region comprising the nucleotide sequence of SEQ ID NO: 6559; a GFAP promoter comprising the nucleotide sequence of SEQ ID NO: 6568; a human beta-globin intron region comprising the nucleotide sequence comprising the nucleotide sequence of SEQ ID NO: 6595; a heavy chain signal sequence comprising the nucleotide sequence of SEQ ID NO: 5157, a heavy chain variable region comprising the nucleotide sequence of SEQ ID NO: 5129, and heavy chain constant region comprising the nucleotide sequence of SEQ ID NO: 5017; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159, a light chain variable region comprising the nucleotide sequence of SEQ ID NO: 5133 and light chain constant region comprising the nucleotide sequence of SEQ ID NO: 5021; a rabbit globin polyadenylation sequence comprising the nucleotide sequence of SEQ ID NO: 6590; and a 3′ ITR sequence region comprising the nucleotide sequence of SEQ ID NO: 6561; or a sequence substantially identical (e.g., having at least 70%, 75%, 80%, 85%, 90%, 95%, or 99% sequence identity) to any of the aforesaid sequences. In some embodiments, disclosed herein is an antibody molecule encoded by the genetic element comprising the nucleotide sequence of SEQ ID NO: 5169 or a sequence substantially identical (e.g., having at least 70%, 75%, 80%, 85%, 90%, 95%, or 99% sequence identity) thereto.

In some embodiments, a genetic element comprising the sequence of SEQ ID NO: 5169, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5137, a HC CDR2 amino acid sequence of SEQ ID NO: 5138, and an HC CDR3 amino acid sequence of SEQ ID NO: 5139; and a light chain variable region comprising an LC CDR1 amino acid sequence of SEQ ID NO: 5143, an LC CDR2 amino acid sequence of SEQ ID NO: 5144, and an LC CDR3 amino acid sequence of SEQ ID NO: 5145. In some embodiments, a genetic element comprising the sequence of SEQ ID NO: 5169, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5140, a HC CDR2 amino acid sequence of SEQ ID NO: 5141, and an HC CDR3 amino acid sequence of SEQ ID NO: 5142; and a light chain variable region comprising an LC CDR1 amino acid sequence of SEQ ID NO: 5146, an LC CDR2 amino acid sequence of SEQ ID NO: 5147, and an LC CDR3 amino acid sequence of SEQ ID NO: 5148.

In some embodiments, a genetic element comprising the sequence of SEQ ID NO: 5169, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5130 and/or a light chain variable region comprising the amino acid sequence of SEQ ID NO: 5134; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5169, or a sequence substantially identical (e.g., having at least 70%, 75%, 80%, 85%, 90%, 95%, or 99% sequence identity) thereto, encodes an antibody molecule comprising a heavy chain comprising the amino acid sequence of SEQ ID NO: 5132 and/or a light chain comprising the amino acid sequence of SEQ ID NO: 5136, or a sequence substantially identical (e.g., having at least 70%, 75%, 80%, 85%, 90%, 95%, or 99% sequence identity) to the any of the aforesaid sequences.

TABLE 24
Sequence Regions in ITR to ITR Sequences

HER-62 (SEQ ID NO: 5188)

| Sequence | Region | Region | Position in |
|---|---|---|---|
| Regions | SEQ ID NO | length (nt) | SEQ ID NO: 5188 |
| 5′ITR | 6559 | 130 | 1-130 |
| CB promoter | 6566 | 260 | 590-849 |
| Exon | 6573 | 134 | 877-1010 |
| Intron | 6578 | 32 | 1011-1042 |
| Intron | 6580 | 347 | 1043-1389 |
| Exon | 6576 | 53 | 1390-1442 |
| Signal (H5) | 5157 | 57 | 1467-1523 |
| VH | 5257 | 357 | 1524-1880 |
| Linker | 5347 | 15 | 1881-1925 |
| VL | 5259 | 321 | 1926-2246 |
| Constant | 5279 | 696 | 2247-2942 |
| PolyA | 6590 | 127 | 2985-3111 |
| 3′ ITR | 6561 | 130 | 3155-3284 |

In some embodiments, the AAV particle comprises a genetic element comprising the nucleotide sequence of SEQ ID NO: 5188 (HER-62), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5188, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5188, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5257, a linker region comprising the nucleotide sequence of SEQ ID NO: 5347; a light chain variable region comprising the nucleotide sequence of SEQ ID NO: 5259, and light chain constant region comprising the nucleotide sequence of SEQ ID NO: 5279; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5188 or a sequence substantially identical (e.g., having at least 70%, 75%, 80%, 85%, 90%, 95%, or 99% sequence identity) thereto.

In some embodiments, a genetic element comprising the sequence of SEQ ID NO: 5188, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5305, a HC CDR2 amino acid sequence of SEQ ID NO: 5306, and an HC CDR3 amino acid sequence of SEQ ID NO: 5307; and a light chain variable region comprising an LC CDR1 amino acid sequence of SEQ ID NO: 5308, an LC CDR2 amino acid sequence of SEQ ID NO: 5309, and an LC CDR3 amino acid sequence of SEQ ID NO: 5310.

In some embodiments, a genetic element comprising the sequence of SEQ ID NO: 5188, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5258 and/or a light chain variable region comprising the amino acid sequence of SEQ ID NO: 5260; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5188, or a sequence substantially identical (e.g., having at least 70%, 75%, 80%, 85%, 90%, 95%, or 99% sequence identity) thereto, encodes an antibody molecule comprising an scFv comprising the amino acid sequence of SEQ ID NO: 5349 and/or an Fc region comprising the amino acid sequence of SEQ ID NO: 5278; or a sequence substantially identical (e.g., having at least 70%, 75%, 80%, 85%, 90%, 95%, or 99% sequence identity) to the any of the aforesaid sequences.

TABLE 25
Sequence Regions in ITR to ITR Sequences

| | HER-73 (SEQ ID NO: 5189) | | |
|---|---|---|---|
| | Region | Region length | Position in |
| Sequence Regions | SEQ ID NO | (nt) | SEQ ID NO: 5189 |
| 5′ ITR | 6559 | 130 | 1-130 |
| CB promoter | 6566 | 260 | 590-849 |
| Exon | 6573 | 134 | 877-1010 |
| Intron | 6578 | 32 | 1011-1042 |
| Intron | 6580 | 347 | 1043-1389 |
| Exon | 6576 | 53 | 1390-1442 |
| Signal | 5157 | 57 | 1467-1523 |
| VH | 5261 | 360 | 1524-1883 |
| Constant | 5215 | 993 | 1884-2876 |
| Linker | 6557 | 12 | 2877-2888 |
| Linker | 6558 | 54 | 2889-2942 |
| Signal | 5159 | 66 | 2943-3008 |
| VH | 6552 | 360 | 3009-3368 |
| Linker | 5347 | 45 | 3369-3413 |
| VL | 5265 | 321 | 3414-3734 |
| Linker | GGCTCT | 6 | 3735-3740 |
| VL | 5353 | 339 | 3741-4079 |
| Constant | 5217 | 321 | 4080-4400 |
| PolyA | 6590 | 127 | 4443-4569 |
| 3′ ITR | 6561 | 130 | 4613-4742 |

In some embodiments, the AAV particle comprises a genetic element comprising the nucleotide sequence of SEQ ID NO: 5189 (HER-73), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5189, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5189, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5261, a heavy chain constant region comprising the nucleotide sequence of SEQ ID NO: 5215; a furin cleavage site comprising the nucleotide sequence of SEQ ID NO: 6557; a T2A linker comprising the nucleotide sequence of SEQ ID NO: 6558; a light chain signal sequence comprising the nucleotide sequence of SEQ ID NO: 5159; a heavy chain variable region comprising the nucleotide sequence of SEQ ID NO: 6552, a linker region comprising the nucleotide sequence of SEQ ID NO: 5347, and a light chain variable region comprising the nucleotide sequence of SEQ ID NO: 5265; a linker comprising the nucleotide sequence of GGCTCT; a light chain variable region comprising the nucleotide sequence of SEQ ID NO: 5353, and a light chain constant region comprising the nucleotide sequence of SEQ ID NO: 5217; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5189 or a sequence substantially identical (e.g., having at least 70%, 75%, 80%, 85%, 90%, 95%, or 99% sequence identity) thereto.

In some embodiments, a genetic element comprising the sequence of SEQ ID NO: 5189, or a sequence substantially identical (e.g., having at least 70%, 75%, 80%, 85%, 90%, 95%, or 99% sequence identity) thereto, encodes a multispecific, e.g., bispecific or biparatopic, antibody molecule, comprising a first antigen binding domain that binds domain I of a HER2 antigen and a second antigen binding domain that binds domain IV of a HER2 antigen. In some embodiments, the first antigen binding domain comprises a full antibody, e.g., an IgG antibody. In some embodiments, the second antigen binding domain comprises an scFv. In some embodiments, the first antigen binding domain comprises a heavy chain variable region comprising a HC CDR1 amino acid sequence of SEQ ID NO: 5358, a HC CDR2 amino acid sequence of SEQ ID NO: 5359, and an HC CDR3 amino acid sequence of SEQ ID NO: 5360; and a light chain variable region comprising an LC CDR1 amino acid sequence of SEQ ID NO: 5355, an LC CDR2 amino acid sequence of SEQ ID NO: 5356, and an LC CDR3 amino acid sequence of SEQ ID NO: 5357. In some embodiments, the second antigen binding domain comprises a HC CDR1 amino acid sequence of SEQ ID NO: 5361, a HC CDR2 amino acid sequence of SEQ ID NO: 5362, and an HC CDR3 amino acid sequence of SEQ ID NO: 5363; and a light chain variable region comprising an LC CDR1 amino acid sequence of SEQ ID NO: 5317, an LC CDR2 amino acid sequence of SEQ ID NO: 5318, and an LC CDR3 amino acid sequence of SEQ ID NO: 5319.

In some embodiments, a genetic element comprising the sequence of SEQ ID NO: 5189, or a sequence substantially identical (e.g., having at least 70%, 75%, 80%, 85%, 90%, 95%, or 99% sequence identity) thereto, encodes a multispecific antibody molecule comprising a first chain, which comprises, from the N-terminus to the C-terminus, a first anti-HER2 VH comprising the amino acid sequence of SEQ ID NO: 5262, and a heavy chain constant region comprising the amino acid sequence of SEQ ID NO: 5216; and a second chain, which comprises from the N-terminus to the C-terminus, a second anti-HER2 VH comprising the amino sequence of 5290, a (G4S)3 linker (SEQ ID NO: 4537), a first anti-HER2 VL comprising the amino acid sequence of SEQ ID NO: 5266, a (GS) linker, a second anti-HER2 VL comprising the amino acid sequence of SEQ ID NO: 5354, and a light chain constant region (CL) comprising the amino acid sequence of SEQ ID NO: 5218; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5189, or a sequence substantially identical (e.g., having at least 70%, 75%, 80%, 85%, 90%, 95%, or 99% sequence identity) thereto, encodes a multispecific antibody molecule comprising a first chain comprising a heavy chain comprising the amino acid sequence of SEQ ID NO: 5264; and a second chain comprising an scFv comprising the amino acid sequence of SEQ ID NO: 5351, fused to a light chain comprising the amino acid sequence of SEQ ID NO: 5268; or a sequence substantially identical (e.g., having at least 70%, 75%, 80%, 85%, 90%, 95%, or 99% sequence identity) to the any of the aforesaid sequences. In some embodiments, a genetic element comprising the sequence of SEQ ID NO: 5189, or a sequence substantially identical (e.g., having at least 70%, 75%, 80%, 85%, 90%, 95%, or 99% sequence identity) thereto, encodes a multispecific antibody molecule comprising a first chain comprising the amino acid sequence of SEQ ID NO: 5264, or an amino acid sequence substantially identical (e.g., having at least 70%, 75%, 80%, 85%, 90%, 95%, or 99% sequence identity) thereto; and/or a second chain comprising the amino acid sequence of SEQ ID NO: 5365, or an amino acid sequence substantially identical (e.g., having at least 70%, 75%, 80%, 85%, 90%, 95%, or 99% sequence identity) thereto.

TABLE 26
Sequence Regions in ITR to ITR Sequences

| | HER-75 (SEQ ID NO: 5190) | | | | | | |
|---|---|---|---|---|---|---|---|
| | Region | Region | Position in | | HER-93 (SEQ ID NO: 6611) | | |
| Sequence | SEQ ID | length | SEQ ID NO: | Sequence | SEQ ID | Region | Position in SEQ |
| Regions | NO | (nt) | 5190 | Regions | NO | length (nt) | ID NO: 6611 |
| 5′ ITR | 6559 | 130 | 1-130 | 5′ ITR | 6559 | 130 | 1-130 |
| CB | 6566 | 260 | 590-849 | GFAP | 6568 | 699 | 216-914 |
| promoter | | | | promoter | | | |
| Exon | 6573 | 134 | 877-1010 | Exon | 6573 | 134 | 935-1068 |
| Intron | 6578 | 32 | 1011-1042 | Intron | 6578 | 32 | 1069-1100 |
| Intron | 6580 | 347 | 1043-1389 | Intron | 6580 | 347 | 1101-1447 |
| Exon | 6576 | 53 | 1390-1442 | Exon | 6576 | 53 | 1448-1500 |
| Signal | 5032 | 57 | 1467-1523 | Signal | 5032 | 57 | 1525-1581 |
| VH | 5269 | 360 | 1524-1883 | VH | 5269 | 360 | 1582-1941 |
| Constant | 5219 | 990 | 1884-2873 | Constant | 5219 | 990 | 1942-2931 |
| Linker | 6557 | 12 | 2874-2885 | Linker | 6557 | 12 | 2932-2943 |
| Linker | 6558 | 54 | 2886-2939 | Linker | 6558 | 54 | 2944-2997 |
| Signal | 5159 | 66 | 2940-3005 | Signal | 5159 | 66 | 2998-3063 |
| VL | 5273 | 321 | 3006-3326 | VL | 5273 | 321 | 3064-3384 |
| Constant | 5221 | 321 | 3327-3647 | Constant | 5221 | 321 | 3385-3705 |
| PolyA | 6590 | 127 | 3690-3816 | PolyA | 6590 | 127 | 3748-3874 |
| 3′ ITR | 6561 | 130 | 3860-3989 | 3′ ITR | 6561 | 130 | 3918-4047 |

In some embodiments, the AAV particle comprises a genetic element comprising the nucleotide sequence of SEQ ID NO: 5190 (HER-75), SEQ ID NO: 6500 (HER-88), SEQ ID NO: 6501 (HER-89), SEQ ID NO: 6502 (HER-90), SEQ ID NO: 6503 (HER-91), SEQ ID NO: 6504 (HER-92), SEQ ID NO: 6611 (HER-93), or a nucleotide sequence substantially identical (e.g., having at least 70%, 75%, 80%, 85%, 90%, 95%, 99% or 100%, sequence identity) thereto. In some embodiments, the AAV particle comprises a genetic element comprising the nucleotide sequence of SEQ ID NO: 5190 (HER-75), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5190, 6500, 6501, 6502, 6503, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5190, 6500, 6501, 6502, 6503, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566 or a GFAP promoter comprising the nucleotide sequence of SEQ ID NO: 6568; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5032, a heavy chain variable region comprising the nucleotide sequence of SEQ ID NO: 5269, 6512, 6517, 6522, 6527, or 6532, and heavy chain constant region comprising the nucleotide sequence of SEQ ID NO: 5219; a furin cleavage site comprising the nucleotide sequence of SEQ ID NO: 6557; a T2A linker comprising the nucleotide sequence of SEQ ID NO: 6558; a light chain signal sequence comprising the nucleotide sequence of SEQ ID NO: 5159; a light chain variable region comprising the nucleotide sequence of SEQ ID NO: 5273, and light chain constant region comprising the nucleotide sequence of SEQ ID NO: 5221; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5190 or a sequence substantially identical (e.g., having at least 70%, 75%, 80%, 85%, 90%, 95%, or 99% sequence identity) thereto. In some embodiments, the nucleotide sequence encoding the heavy chain constant region comprises a modification, e.g., a substitution, at position 897 (e.g., G897A), numbered according to 5219. In some embodiments, the nucleotide sequence encoding the heavy chain constant region comprises a G to A substitution at position 897 (G897A), numbered according to 5219. In some embodiments, the nucleotide sequence encoding the light chain variable region comprises a modification at position 69 (e.g., C69T), numbered according to SEQ ID NO: 5273. In some embodiments, the nucleotide sequence encoding the light chain variable region comprises a C to T substitution at position 69 (e.g., C69T), numbered according to SEQ ID NO: 5273.

In some embodiments, a genetic element comprising the sequence of SEQ ID NO: 5190, 6500, 6501, 6502, 6503, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5281, a HC CDR2 amino acid sequence of SEQ ID NO: 5282, and an HC CDR3 amino acid sequence of SEQ ID NO: 5283, 6510, 6515, 6520, 6525 or 6530; and a light chain variable region comprising an LC CDR1 amino acid sequence of SEQ ID NO: 5287, an LC CDR2 amino acid sequence of SEQ ID NO: 5288, and an LC CDR3 amino acid sequence of SEQ ID NO: 5289. In some embodiments, a genetic element comprising the sequence of SEQ ID NO: 5190, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5284, a HC CDR2 amino acid sequence of SEQ ID NO: 5285, and an HC CDR3 amino acid sequence of SEQ ID NO: 5286; and a light chain variable region comprising an LC CDR1 amino acid sequence of SEQ ID NO: 5287, an LC CDR2 amino acid sequence of SEQ ID NO: 5291, and an LC CDR3 amino acid sequence of SEQ ID NO: 5292.

In some embodiments, a genetic element comprising the sequence of SEQ ID NO: 5190, 6500, 6501, 6502, 6503, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5270, 6511, 6516, 6521, 6526 or 6531 and/or a light chain variable region comprising the amino acid sequence of SEQ ID NO: 5274; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5190, 6500, 6501, 6502, 6503, or a sequence substantially identical (e.g., having at least 70%, 75%, 80%, 85%, 90%, 95%, or 99% sequence identity) to the any of the aforesaid sequences, encodes an antibody molecule comprising a heavy chain comprising the amino acid sequence of SEQ ID NO: 5272, 6513, 6518, 6523, 6528, 6533 and/or a light chain comprising the amino acid sequence of SEQ ID NO: 5276; or a sequence substantially identical (e.g., having at least 70%, 75%, 80%, 85%, 90%, 95%, or 99% sequence identity) to the any of the aforesaid sequences.

TABLE 40
Sequence Regions in ITR to ITR Sequences

| | HER-77 (SEQ ID NO: 5343) | | |
|---|---|---|---|
| Sequence | Region | Region length | Position in |
| Regions | SEQ ID NO | (nt) | SEQ ID NO: 5343 |
| 5′ITR | 6559 | 130 | 1-130 |
| CB promoter | 6566 | 260 | 590-849 |
| Exon | 6573 | 134 | 877-1010 |
| Intron | 6578 | 32 | 1011-1042 |
| Intron | 6580 | 347 | 1043-1389 |
| Exon | 6576 | 53 | 1390-1442 |
| Signal | 5157 | 57 | 1467-1523 |
| VH | 5330 | 360 | 1524-1883 |
| Constant | 5344 | 990 | 1884-2873 |
| Linker | 1724 | 12 | 2874-2885 |
| Linker | 1726 | 54 | 2886-2939 |
| Signal | 5159 | 66 | 2940-3005 |
| VL | 5340 | 321 | 3006-3326 |
| Constant | 5346 | 321 | 3327-3647 |
| PolyA | 6590 | 127 | 3690-3816 |
| 3′ ITR | 6561 | 130 | 3860-3989 |

In some embodiments, the AAV particle comprises a genetic element comprising the nucleotide sequence of SEQ ID NO: 5346 (HER-77), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5346, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5346, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5330, and heavy chain constant region comprising the nucleotide sequence of SEQ ID NO: 5344; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5340; a light chain variable region comprising the nucleotide sequence of SEQ ID NO: 5346, and light chain constant region comprising the nucleotide sequence of SEQ ID NO: 5221; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5343 or a sequence substantially identical (e.g., having at least 70%, 75%, 80%, 85%, 90%, 95%, or 99% sequence identity) thereto.

In some embodiments, a genetic element comprising the sequence of SEQ ID NO: 5343, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5281, a HC CDR2 amino acid sequence of SEQ ID NO: 5282, and an HC CDR3 amino acid sequence of SEQ ID NO: 5283; and a light chain variable region comprising an LC CDR1 amino acid sequence of SEQ ID NO: 5287, an LC CDR2 amino acid sequence of SEQ ID NO: 5288, and an LC CDR3 amino acid sequence of SEQ ID NO: 5289. In some embodiments, a genetic element comprising the sequence of SEQ ID NO: 5343, or a sequence substantially identical (e.g., having at least 70%, 75%, 80%, 85%, 90%, 95%, or 99% sequence identity) thereto, encodes an antibody molecule comprising a heavy chain variable region comprising a HC CDR1 amino acid sequence of SEQ ID NO: 5284, a HC CDR2 amino acid sequence of SEQ ID NO: 5285, and an HC CDR3 amino acid sequence of SEQ ID NO: 5286; and a light chain variable region comprising an LC CDR1 amino acid sequence of SEQ ID NO: 5287, an LC CDR2 amino acid sequence of SEQ ID NO: 5291, and an LC CDR3 amino acid sequence of SEQ ID NO: 5292.

In some embodiments, a genetic element comprising the sequence of SEQ ID NO: 5343, or a sequence substantially identical (e.g., having at least 70%, 75%, 80%, 85%, 90%, 95%, or 99% sequence identity) thereto, encodes an antibody molecule comprising a heavy chain variable region comprising the amino acid sequence of SEQ ID NO: 5329 and/or a light chain variable region comprising the amino acid sequence of SEQ ID NO: 5339; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5343, or a sequence substantially identical (e.g., having at least 70%, 75%, 80%, 85%, 90%, 95%, or 99% sequence identity) to the any of the aforesaid sequences, encodes an antibody molecule comprising a heavy chain comprising the amino acid sequence of SEQ ID NO: 5331 and/or a light chain comprising the amino acid sequence of SEQ ID NO: 5341; or a sequence substantially identical (e.g., having at least 70%, 75%, 80%, 85%, 90%, 95%, or 99% sequence identity) to the any of the aforesaid sequences.

TABLE 42
Sequence Regions in ITR to ITR Sequences

| | HER-82 (SEQ ID NO: 5374) | | |
|---|---|---|---|
| | Region | Region length | Position in |
| Sequence Regions | SEQ ID NO | (nt) | SEQ ID NO: 5374 |
| 5′ ITR | 6559 | 130 | 1-130 |
| CB promoter | 6566 | 260 | 590-849 |
| Exon | 6573 | 134 | 877-1010 |
| Intron | 6578 | 32 | 1011-1042 |
| Intron | 6580 | 347 | 1043-1389 |
| Exon | 6576 | 53 | 1390-1442 |
| Signal | 5157 | 57 | 1467-1523 |
| VH | 5002 | 360 | 1524-1883 |
| Constant | 5017 | 990 | 1884-2873 |
| Linker | 1724 | 12 | 2874-2885 |
| Linker | 1726 | 54 | 2886-2939 |
| Signal | 5159 | 66 | 2940-3005 |
| DARPIN | 5371 | 471 | 3006-3476 |
| Linker | 5347 | 45 | 3477-3521 |
| VL | 5005 | 321 | 3522-3842 |
| Constant | 5007 | 321 | 3843-4163 |
| PolyA | 6590 | 127 | 4206-4332 |
| 3′ ITR | 6561 | 130 | 4376-4505 |

In some embodiments, the AAV particle comprises a genetic element comprising the nucleotide sequence of SEQ ID NO: 5374 (HER-82), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5374, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5374, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5002, a heavy chain constant region comprising the nucleotide sequence of SEQ ID NO: 5017; a furin cleavage site comprising the nucleotide sequence of SEQ ID NO: 1724; a T2A linker comprising the nucleotide sequence of SEQ ID NO: 1726; a light chain signal sequence comprising the nucleotide sequence of SEQ ID NO: 5159; a DARPIN molecule comprising the nucleotide sequence of SEQ ID NO: 5371; a linker region comprising the nucleotide sequence of SEQ ID NO: 5347; a light chain variable region comprising the nucleotide sequence of SEQ ID NO: 5005; a light chain constant region comprising the nucleotide sequence of SEQ ID NO: 5007; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5374 or a sequence substantially identical (e.g., having at least 70%, 75%, 80%, 85%, 90%, 95%, or 99% sequence identity) thereto.

In some embodiments, a genetic element comprising the sequence of SEQ ID NO: 5374, or a sequence substantially identical (e.g., having at least 70%, 75%, 80%, 85%, 90%, 95%, or 99% sequence identity) thereto, encodes a multispecific, e.g., bispecific or biparatopic, antibody molecule, comprising a first antigen binding domain that binds domain IV of a HER2 antigen and a second antigen binding domain that binds domain I of a HER2 antigen. In some embodiments, the first antigen binding domain comprises a full antibody. In some embodiments, the second antigen binding domain comprises a DARPIN molecule. In some embodiments, the first antigen binding domain comprises a heavy chain variable region comprising a HC CDR1 amino acid sequence of SEQ ID NO: 5361, a HC CDR2 amino acid sequence of SEQ ID NO: 5362, and an HC CDR3 amino acid sequence of SEQ ID NO: 5363; and a light chain variable region comprising an LC CDR1 amino acid sequence of SEQ ID NO: 5317, an LC CDR2 amino acid sequence of SEQ ID NO: 5318, and an LC CDR3 amino acid sequence of SEQ ID NO: 5319. In some embodiments, the second antigen binding domain comprises the amino acid sequence of SEQ ID NO: 5370, or an amino acid sequence substantially identical (e.g., having at least 70%, 75%, 80%, 85%, 90%, 95%, or 99% sequence identity) thereto.

In some embodiments, a genetic element comprising the sequence of SEQ ID NO: 5374, or a sequence substantially identical (e.g., having at least 70%, 75%, 80%, 85%, 90%, 95%, or 99% sequence identity) thereto, encodes a multispecific antibody molecule, comprising: a first chain, comprising from the N-terminus to the C-terminus, an anti-HER2 VH comprising the amino sequence comprising the amino acid sequence of SEQ ID NO: 5001, and a heavy chain constant region comprising the amino acid sequence of SEQ ID NO: 5018; and a second chain, comprising from the N-terminus to the C-terminus, a DARPIN molecule comprising the amino acid sequence of SEQ ID NO: 5370, a (G4S)3 linker (SEQ ID NO: 4537), an anti-HER2 VL comprising the amino acid sequence of SEQ ID NO: 5006, and a light chain constant region (CL) comprising the amino acid sequence of SEQ ID NO: 5008; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5374, or a sequence substantially identical (e.g., having at least 70%, 75%, 80%, 85%, 90%, 95%, or 99% sequence identity) thereto, encodes a multispecific antibody molecule, comprising a first chain comprising the amino acid sequence of SEQ ID NO: 5368; and a second chain comprising the amino acid sequence of SEQ ID NO: 5370, a (G4S)3 linker (SEQ ID NO: 4537), and the amino acid sequence comprising of SEQ ID NO: 5063; or a sequence substantially identical (e.g., having at least 70%, 75%, 80%, 85%, 90%, 95%, or 99% sequence identity) to the any of the aforesaid sequences. In some embodiments, a genetic element comprising the sequence of SEQ ID NO: 5374, or a sequence substantially identical (e.g., having at least 70%, 75%, 80%, 85%, 90%, 95%, or 99% sequence identity) thereto, encodes a multispecific antibody molecule, comprising a first chain comprising the amino acid sequence of SEQ ID NO: 5368, and a second chain comprising the amino acid sequence of SEQ ID NO: 5372; or a sequence substantially identical (e.g., having at least 70%, 75%, 80%, 85%, 90%, 95%, or 99% sequence identity) to the any of the aforesaid sequences.

TABLE 43
Sequence Regions in ITR to ITR Sequences

| | HER-78 (SEQ ID NO: 5375) | | |
|---|---|---|---|
| | Region | Region length | Position in |
| Sequence Regions | SEQ ID NO | (nt) | SEQ ID NO: 5375 |
| 5′ ITR | 6559 | 130 | 1-130 |
| CB promoter | 6566 | 260 | 590-849 |
| Exon | 6573 | 134 | 877-1010 |
| Intron | 6578 | 32 | 1011-1042 |
| Intron | 6580 | 347 | 1043-1389 |
| Exon | 6576 | 53 | 1390-1442 |
| Signal | 5157 | 57 | 1467-1523 |
| VH | 5261 | 360 | 1524-1883 |
| Constant | 5219 | 990 | 1884-2873 |
| Linker | 6557 | 12 | 2874-2885 |
| Linker | 6558 | 54 | 2886-2939 |
| Signal | 5159 | 66 | 2940-3005 |
| VH | 6552 | 360 | 3006-3365 |
| Linker | 5347 | 45 | 3366-3410 |
| VL | 5265 | 321 | 3411-3731 |
| Linker | GGCTCT | 6 | 3732-3737 |
| VL | 5353 | 339 | 3738-4076 |
| Constant | 5217 | 321 | 4077-4397 |
| PolyA | 6590 | 127 | 4440-4566 |
| 3′ ITR | 6561 | 130 | 4610-4739 |

In some embodiments, the AAV particle comprises a genetic element comprising the nucleotide sequence of SEQ ID NO: 5375 (HER-78), 6505 (HER-97), 6506 (HER-98), 6507 (HER-99), 6508 (HER-100), 6509 (HER-101), or a nucleotide sequence substantially identical (e.g., having at least 70%, 75%, 80%, 85%, 90%, 95%, 99% or 100%, sequence identity) thereto. In some embodiments, the genetic element comprises the nucleotide sequence of SEQ ID NO: 5375, 6505, 6506, 6507, 6508, 6509, or a nucleotide sequence substantially identical (e.g., having at least 70%, 75%, 80%, 85%, 90%, 95%, 99% or 100%, sequence identity) thereto. In some embodiments, the genetic element comprising the nucleotide sequence of SEQ ID NO: 5375, 6505, 6506, 6507, 6508, or 6509, comprises in 5′ to 3′ order: a 5′ inverted terminal repeat (ITR) sequence region comprising the nucleotide sequence of SEQ ID NO: 6559; a CB promoter comprising the nucleotide sequence of SEQ ID NO: 6566; an ie1 exon 1 region comprising the nucleotide sequence of SEQ ID NO: 6573; a human beta-globin intron region comprising the nucleotide sequence of SEQ ID NO: 6578; a second human beta-globin intron region comprising the nucleotide sequence of SEQ ID NO: 6580; a human beta-globin exon region comprising the nucleotide sequence of SEQ ID NO: 6576; a heavy chain signal sequence comprising the nucleotide sequence of SEQ ID NO: 5157, a heavy chain variable region comprising the nucleotide sequence of SEQ ID NO: 5261, a heavy chain constant region comprising the nucleotide sequence of SEQ ID NO: 5219; a furin cleavage site comprising the nucleotide sequence of SEQ ID NO: 6557; a T2A linker comprising the nucleotide sequence of SEQ ID NO: 6558; a light chain signal sequence comprising the nucleotide sequence of SEQ ID NO: 5159; a heavy chain variable region comprising the nucleotide sequence of SEQ ID NO: 6552, 6537, 6540, 6543, 6546, or 6549, a linker region comprising the nucleotide sequence of SEQ ID NO: 5347, and a light chain variable region comprising the nucleotide sequence of SEQ ID NO: 5265; a linker comprising the nucleotide sequence of GGCTCT; a light chain variable region comprising the nucleotide sequence of SEQ ID NO: 5353, and a light chain constant region comprising the nucleotide sequence of SEQ ID NO: 5217; a rabbit globin polyadenylation sequence comprising the nucleotide sequence of SEQ ID NO: 6590; and a 3′ ITR sequence region comprising the nucleotide sequence of SEQ ID NO: 6561; or a nucleotide sequence substantially identical (e.g., having at least 70%, 75%, 80%, 85%, 90%, 95%, 99% or 100%, sequence identity) to any of the aforesaid sequences. In some embodiments, disclosed herein is an antibody molecule encoded by the genetic element comprising the nucleotide sequence of SEQ ID NO: 5375, 6505, 6506, 6507, 6508, 6509, or a sequence substantially identical (e.g., having at least 70%, 75%, 80%, 85%, 90%, 95%, or 99% sequence identity) thereto.

In some embodiments, a genetic element comprising the sequence of SEQ ID NO: 5375, 6505, 6506, 6507, 6508, 6509, or a sequence substantially identical (e.g., having at least 70%, 75%, 80%, 85%, 90%, 95%, or 99% sequence identity) thereto, encodes a multispecific, e.g., bispecific or biparatopic, antibody molecule, comprising a first antigen binding domain that binds domain I of a HER2 antigen and a second antigen binding domain that binds domain IV of a HER2 antigen. In some embodiments, the first antigen binding domain comprises a full antibody. In some embodiments, the second antigen binding domain comprises an scFv. In some embodiments, the first antigen binding domain comprises a heavy chain variable region comprising a HC CDR1 amino acid sequence of SEQ ID NO: 5358, a HC CDR2 amino acid sequence of SEQ ID NO: 5359, and an HC CDR3 amino acid sequence of SEQ ID NO: 5360, 6535, 6538, 6541, 6544, or 6547; and a light chain variable region comprising an LC CDR1 amino acid sequence of SEQ ID NO: 5355, an LC CDR2 amino acid sequence of SEQ ID NO: 5356, and an LC CDR3 amino acid sequence of SEQ ID NO: 5357. In some embodiments, the second antigen binding domain comprises a HC CDR1 amino acid sequence of SEQ ID NO: 5361, a HC CDR2 amino acid sequence of SEQ ID NO: 5362, and an HC CDR3 amino acid sequence of SEQ ID NO: 5363; and a light chain variable region comprising an LC CDR1 amino acid sequence of SEQ ID NO: 5317, an LC CDR2 amino acid sequence of SEQ ID NO: 5318, and an LC CDR3 amino acid sequence of SEQ ID NO: 5319.

In some embodiments, a genetic element comprising the sequence of SEQ ID NO: 5375, 6505, 6506, 6507, 6508, 6509, or a sequence substantially identical (e.g., having at least 70%, 75%, 80%, 85%, 90%, 95%, or 99% sequence identity) thereto, encodes a multispecific antibody molecule comprising a first chain, which comprises, from the N-terminus to the C-terminus, a first anti-HER2 VH comprising the amino acid sequence of SEQ ID NO: 5262, and a heavy chain constant region comprising the amino acid sequence of SEQ ID NO: 5220; and a second chain, which comprises from the N-terminus to the C-terminus, a second anti-HER2 VH comprising the amino sequence of SEQ ID NO: 5290, 6536, 6539, 6542, 6545, or 6548, a (G4S)3 linker (SEQ ID NO: 4537), a first anti-HER2 VL comprising the amino acid sequence of SEQ ID NO: 5266, a (GS) linker, a second anti-HER2 VL comprising the amino acid sequence of SEQ ID NO: 5354, and a light chain constant region (CL) comprising the amino acid sequence of SEQ ID NO: 5218; or a sequence substantially identical (e.g., having at least 70%, 75%, 80%, 85%, 90%, 95%, or 99% sequence identity) to the any of the aforesaid sequences.

In some embodiments, a genetic element comprising the sequence of SEQ ID NO: 5375, or a sequence substantially identical (e.g., having at least 70%, 75%, 80%, 85%, 90%, 95%, or 99% sequence identity) thereto, encodes a multispecific antibody molecule comprising a first chain comprising a heavy chain comprising the amino acid sequence of SEQ ID NO: 5376; and a second chain comprising an scFv comprising the amino acid sequence of SEQ ID NO: 5351, fused to a light chain comprising the amino acid sequence of SEQ ID NO: 5268; or a sequence substantially identical (e.g., having at least 70%, 75%, 80%, 85%, 90%, 95%, or 99% sequence identity) to the any of the aforesaid sequences. In some embodiments, a genetic element comprising the sequence of SEQ ID NO: 5375, or a sequence substantially identical (e.g., having at least 70%, 75%, 80%, 85%, 90%, 95%, or 99% sequence identity) thereto, encodes a multispecific antibody molecule comprising a first chain comprising the amino acid sequence of SEQ ID NO: 5376, or an amino acid sequence substantially identical (e.g., having at least 70%, 75%, 80%, 85%, 90%, 95%, or 99% sequence identity) thereto; and/or a second chain comprising the amino acid sequence of SEQ ID NO: 5365, or an amino acid sequence substantially identical (e.g., having at least 70%, 75%, 80%, 85%, 90%, 95%, or 99% sequence identity) thereto.

In some embodiment, the AAV particle comprises an AAV vector comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity). In some embodiments, the AAV vector further comprises a nucleotide sequence encoding a capsid protein, e.g., a structural protein. In some embodiments, the capsid protein comprises a VP1 polypeptide, a VP2 polypeptide, and/or a VP3 polypeptide. In some embodiments, the VP1 polypeptide, the VP2 polypeptide, and/or the VP3 polypeptide are encoded by at least one Cap gene. In some embodiments, the AAV vector further encodes a Rep protein, e.g., a non-structural protein. In some embodiments, the Rep protein comprises a Rep78 protein, a Rep68, Rep52 protein, and/or a Rep40 protein. In some embodiments, the Rep78 protein, the Rep68 protein, the Rep52 protein, and/or the Rep40 protein are encoded by at least one Rep gene.

In some embodiment, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises, e.g., is packaged in, an AAV capsid polypeptide, e.g., an AAV capsid variant provided in any of Tables 1 or 37-39. For example, the AAV capsid polypeptide, e.g., AAV capsid variant comprises a VOY101 capsid polypeptide, a VOY9P39 capsid polypeptide, a VOY9P33 capsid polypeptide, a AAVPHP.B (PHP.B) capsid polypeptide, a AAVPHP.N (PHP.N) capsid polypeptide, an AAV1 capsid polypeptide, an AAV2 capsid polypeptide, an AAV5 capsid polypeptide, an AAV9 capsid polypeptide, an AAVrh10 capsid polypeptide, or a functional variant thereof.

In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises a VOY101 capsid polypeptide or functional variant thereof. In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising the amino acid sequence of SEQ ID NO: 1, or an amino acid sequence substantially identical (e.g., having at least 70%, 75%, 80%, 85%, 90%, 95% or 99%) thereto.

In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising an amino acid sequence having at least one, two or three modifications, but not more than 30, 20 or 10 modifications of the amino acid sequence of SEQ ID NO: 1.

In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising the amino acid sequence of SEQ ID NO: 138, or a sequence substantially identical (e.g., having at least about 70%, 75%, 80%, 85%, 90%, 92%, 95%, 97%, 98%, or 99% sequence identity) thereto. In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising an amino acid sequence having at least one, two or three modifications, but not more than 30, 20 or 10 modifications of the amino acid sequence of SEQ ID NO: 138. In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, encoded by the nucleotide sequence of SEQ ID NO: 137, or a nucleotide sequence substantially identical (e.g., having at least 70%, 75%, 80%, 85%, 90%, 95% or 99%) thereto. In some embodiments, the capsid protein comprises an amino acid substitution at position K449, e.g., a K449R substitution, numbered according to SEQ ID NO: 138. In some embodiments, the capsid protein comprises an insert comprising the amino acid sequence of TLAVPFK (SEQ ID NO: 1262), wherein the insert is present immediately subsequent to position 588, relative to a reference sequence numbered according to SEQ ID NO:138. In some embodiments, the capsid protein comprises an amino acid other than “A” at position 587 and/or an amino acid other than “Q” at position 588, numbered according to SEQ ID NO: 138. In some embodiments, the capsid protein comprises the amino acid substitution of A587D and/or Q588G, numbered according to SEQ ID NO: 138.

In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising the amino acid sequence of SEQ ID NO: 12, or a sequence substantially identical (e.g., having at least about 70%, 75%, 80%, 85%, 90%, 92%, 95%, 97%, 98%, or 99% sequence identity) thereto. In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising an amino acid sequence having at least one, two or three modifications, but not more than 30, 20 or 10 modifications of the amino acid sequence of SEQ ID NO: 12.

In some embodiments, the AAV particle comprising a genetic element comprising the nucleotide sequence of any of the genetic elements described herein, e.g., as described in Tables 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising the amino acid sequence of SEQ ID NO: 14, or a sequence substantially identical (e.g., having at least about 70%, 75%, 80%, 85%, 90%, 92%, 95%, 97%, 98%, or 99% sequence identity) thereto. In some embodiments, the AAV particle comprising a genetic element comprising the nucleotide sequence of any of the genetic elements described herein, e.g., as described in 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising an amino acid sequence having at least one, two or three modifications, but not more than 30, 20 or 10 modifications of the amino acid sequence of SEQ ID NO: 14.

In some embodiments, the AAV particle comprising a genetic element comprising the nucleotide sequence of any of the genetic elements described herein, e.g., as described in 6, 17-19, 23-26, 40, 42, 43, 68, and 69, or a nucleotide sequence substantially identical (e.g., having at least about 70%, 75%, 80%, 85%, 90%, 92%, 95%, 97%, 98%, or 99% sequence identity) to any of the aforesaid sequences comprises an AAV capsid polypeptide, e.g., an AAV capsid variant, comprising the amino acid sequence of any one of SEQ ID NO: 981 or 982, or an amino acid sequence with at least 80% (e.g., at least about 85, 90, 95, 96, 97, 98, or 99%) sequence identity thereto.

In some embodiments, the AAV particle comprises a genetic element comprising a nucleotide sequence encoding one or more antibodies directed against HER2 or its ligands. Exemplary HER2 antibodies and target ligands have been described in Hudziak et al., Mol. Cell. Biol. 9(3):1165-1172 (1989); Tagliabue et al. Int. J. Cancer 47:933-937 (1991); McKenzie et al. Oncogene 4:543-548 (1989); Maier et al. Cancer Res. 51:5361-5369 (1991); Bacus et al. Molecular Carcinogenesis 3:350-362 (1990); Stancovski et al. PNAS (USA) 88:8691-8695 (1991); Bacus et al. Cancer Research 52:2580-2589 (1992); Xu et al. Int. J. Cancer 53:401-408 (1993); WO94/00136; Kasprzyk et al. Cancer Research 52:2771-2776 (1992); Hancock et al. Cancer Res. 51:4575-4580 (1991); Shawver et al. Cancer Res. 54:1367-1373 (1994); Arteaga et al. Cancer Res. 54:3758-3765 (1994); Harwerth et al. J. Biol. Chem. 267:15160-15167 (1992); U.S. Pat. No. 5,783,186; Klapper et al. Oncogene 14:2099-2109 (1997); WO 98/17797; and U.S. Pat. No. 5,783,186. In one embodiment, the payload region of the AAV particle comprises one or more nucleic acid sequences encoding one or more of the payload antibody polypeptides related to HER2 listed in Table 28 (APA1-APA236; SEQ ID NO: 6000-6235). Exemplary additional payloads related to HER2 are disclosed in Table 28. The antibody sequences for each of these antibodies are disclosed in the reference information column of Table 28, the contents of each are incorporated herein in their entirety.

TABLE 28
Additional Payload Antibodies Related to HER2

| Antibody | Target | Description | Reference |
|---|---|---|---|
| No. | No | No. | Information |
| APA1 | 1 | 1 | U.S Pat. No. 8,802,093 SEQ ID NO: 11 |
| APA2 | 1 | 1 | WO2013096812 SEQ ID NO: 8 |
| APA3 | 1 | 3 | U.S Pat. No. 5,720,954; U.S Pat. No. 5,770,195; |
| | | | U.S Pat. No. 5,772,997; U.S Pat. No. 6,165,464; |
| | | | U.S Pat. No. 6,387,371; U.S Pat. No. 6,399,063 |
| APA4 | 1 | 5 | U.S Pat. No. 8,557,243 |
| APA5 | 1 | 6 | U.S Pat. No. 8,557,243 |
| APA6 | 1 | 7 | U.S Pat. No. 8,557,243 |
| APA7 | 1 | 2 | U.S Pat. No. 8,557,243 |
| APA8 | 1 | 8 | U.S Pat. No. 5,720,954; U.S Pat. No. 5,770,195; |
| | | | U.S Pat. No. 5,772,997; U.S Pat. No. 6,165,464; |
| | | | U.S Pat. No. 6,387,371; U.S Pat. No. 6,399,063 |
| APA9 | 1 | 9 | U.S Pat. No. 5,720,954; U.S Pat. No. 5,770,195; |
| | | | U.S Pat. No. 5,772,997; U.S Pat. No. 6,165,464; |
| | | | U.S Pat. No. 6,387,371; U.S Pat. No. 6,399,063 |
| APA10 | 1 | 10 | U.S Pat. No. 8,802,093 SEQ ID NO: 2 |
| APA11 | 1 | 10 | WO2013096812 SEQ ID NO: 7 |
| APA12 | 1 | 12 | U.S Pat. No. 5,720,954; U.S Pat. No. 5,770,195; |
| | | | U.S Pat. No. 5,772,997; U.S Pat. No. 6,165,464; |
| | | | U.S Pat. No. 6,387,371; U.S Pat. No. 6,399,063 |
| APA13 | 1 | 12 | U.S Pat. No. 5,720,954; U.S Pat. No. 5,770,195; |
| | | | U.S Pat. No. 5,772,997; U.S Pat. No. 6,165,464; |
| | | | U.S Pat. No. 6,387,371; U.S Pat. No. 6,399,063 |
| APA14 | 1 | 13 | U.S Pat. No. 5,720,954; U.S Pat. No. 5,770,195; |
| | | | U.S Pat. No. 5,772,997; U.S Pat. No. 6,165,464; |
| | | | U.S Pat. No. 6,387,371; U.S Pat. No. 6,399,063 |
| APA15 | 1 | 11 | U.S Pat. No. 8,557,243 |
| APA16 | 1 | 14 | U.S Pat. No. 8,557,243 |
| APA17 | 1 | 15 | U.S Pat. No. 8,557,243 |
| APA18 | 1 | 16 | U.S Pat. No. 8,557,243 |
| APA19 | 1 | 1 | US20130089544 SEQ ID NO: 54 |
| APA20 | 1 | 1 | US20130089544 SEQ ID NO: 56 |
| APA21 | 1 | 1 | US20130089544 SEQ ID NO: 57 |
| APA22 | 1 | 1 | US20130089544 SEQ ID NO: 58 |
| APA23 | 1 | 1 | US20130089544 SEQ ID NO: 59 |
| APA24 | 1 | 1 | US20130266564 SEQ ID NO: 10 |
| APA25 | 1 | 1 | US20130266564 SEQ ID NO: 20 |
| APA26 | 1 | 1 | US20130266564 SEQ ID NO: 25 |
| APA27 | 1 | 1 | US20130266564 SEQ ID NO: 26 |
| APA28 | 1 | 1 | US20140010814 SEQ ID NO: 25 |
| APA29 | 1 | 1 | US20140010814 SEQ ID NO: 37 |
| APA30 | 1 | 1 | US20140010814 SEQ ID NO: 39 |
| APA31 | 2 | 1 | US20100136033 SEQ ID NO: 3; |
| | | | US20110129464 SEQ ID NO: 6 |
| APA32 | 2 | 4 | US20160053011 SEQ ID NO: 14 |
| APA33 | 1 | 2 | US20110129464 SEQ ID NO: 2 |
| APA34 | 1 | 2 | US20130089544 SEQ ID NO: 2 |
| APA35 | 1 | 2 | US20130089544 SEQ ID NO: 6 |
| APA36 | 1 | 2 | US20130089544 SEQ ID NO: 10 |
| APA37 | 1 | 2 | US20130089544 SEQ ID NO: 14, |
| | | | 18, 22, 34, 38 |
| APA38 | 1 | 2 | US20130089544 SEQ ID NO: 26 |
| APA39 | 1 | 2 | US20130089544 SEQ ID NO: 30 |
| APA40 | 1 | 2 | US20130089544 SEQ ID NO: 42 |
| APA41 | 1 | 2 | US20130089544 SEQ ID NO: 4 |
| APA42 | 1 | 2 | US20130266564 SEQ ID NO: 8 |
| APA43 | 2 | 2 | US20160089435 SEQ ID NO: 42 |
| APA44 | 2 | 2 | US20080226659 SEQ ID NO: 1; |
| | | | US20090226455 SEQ ID NO: 2; |
| | | | US20100136033 SEQ ID NO: 1 |
| APA45 | 2 | 2 | US20080226659 SEQ ID NO: 2; |
| | | | US20090226455 SEQ ID NO: 4; |
| | | | US20100136033 SEQ ID NO: 2; |
| | | | US20110129464 SEQ ID NO: 4 |
| APA46 | 2 | 2 | US20130171148 SEQ ID NO: 1 |
| APA47 | 2 | 2 | US20130171148 SEQ ID NO: 8 |
| APA48 | 2 | 2 | US20130171148 SEQ ID NO: 15 |
| APA49 | 2 | 2 | US20130171148 SEQ ID NO: 22 |
| APA50 | 2 | 2 | US20130171148 SEQ ID NO: 29 |
| APA51 | 2 | 2 | US20130171148 SEQ ID NO: 36 |
| APA52 | 2 | 2 | US20130171148 SEQ ID NO: 43 |
| APA53 | 2 | 2 | US20130171148 SEQ ID NO: 45 |
| APA54 | 2 | 2 | US20130171148 SEQ ID NO: 47 |
| APA55 | 2 | 2 | US20130171148 SEQ ID NO: 49 |
| APA56 | 2 | 2 | US20130171148 SEQ ID NO: 51 |
| APA57 | 2 | 2 | US20130171148 SEQ ID NO: 53 |
| APA58 | 2 | 2 | US20130171148 SEQ ID NO: 55 |
| APA59 | 2 | 2 | US20130189271 SEQ ID NO: 1, 85 |
| APA60 | 2 | 2 | US20130189271 SEQ ID NO: 8 |
| APA61 | 2 | 2 | US20130189271 SEQ ID NO: 15 |
| APA62 | 2 | 2 | US20130189271 SEQ ID NO: 22 |
| APA63 | 2 | 2 | US20130189271 SEQ ID NO: 35 |
| APA64 | 2 | 2 | US20130189271 SEQ ID NO: 42 |
| APA65 | 2 | 2 | US20130189271 SEQ ID NO: 49 |
| APA66 | 2 | 2 | US20130189271 SEQ ID NO: 63 |
| APA67 | 2 | 2 | US20130189271 SEQ ID NO: 70 |
| APA68 | 2 | 2 | US20130189271 SEQ ID NO: 77, 79 |
| APA69 | 2 | 2 | US20130189271 SEQ ID NO: 81 |
| APA70 | 2 | 2 | US20130189271 SEQ ID NO: 83 |
| APA71 | 2 | 2 | US20130189271 SEQ ID NO: 87 |
| APA72 | 2 | 2 | US20130189271 SEQ ID NO: 89 |
| APA73 | 2 | 2 | US20130189271 SEQ ID NO: 91 |
| APA74 | 2 | 2 | US20130189271 SEQ ID NO: 93 |
| APA75 | 2 | 2 | US20130189271 SEQ ID NO: 95 |
| APA76 | 2 | 2 | US20130189271 SEQ ID NO: 97 |
| APA77 | 2 | 2 | US20130189271 SEQ ID NO: 99 |
| APA78 | 2 | 2 | US20130189271 SEQ ID NO: 101 |
| APA79 | 2 | 2 | US20130189271 SEQ ID NO: 103 |
| APA80 | 2 | 2 | US20130189271 SEQ ID NO: 105 |
| APA81 | 2 | 2 | US20130189271 SEQ ID NO: 29, 107 |
| APA82 | 2 | 2 | US20130189271 SEQ ID NO: 56, 109 |
| APA83 | 2 | 2 | US20130189271 SEQ ID NO: 111 |
| APA84 | 2 | 2 | US20130189271 SEQ ID NO: 113 |
| APA85 | 2 | 2 | US20130189271 SEQ ID NO: 115 |
| APA86 | 2 | 2 | US20130189271 SEQ ID NO: 117 |
| APA87 | 2 | 2 | US20130189271 SEQ ID NO: 119 |
| APA88 | 2 | 2 | US20130189271 SEQ ID NO: 121 |
| APA89 | 2 | 2 | US20130189271 SEQ ID NO: 123 |
| APA90 | 2 | 2 | US20130189271 SEQ ID NO: 125 |
| APA91 | 2 | 2 | US20140356366 SEQ ID NO: 114 |
| APA92 | 2 | 2 | US20150322162 SEQ ID NO: 132 |
| APA93 | 2 | 2 | US20150322162 SEQ ID NO: 133 |
| APA94 | 2 | 2 | US20150322162 SEQ ID NO: 134 |
| APA95 | 2 | 2 | US20150322162 SEQ ID NO: 135 |
| APA96 | 2 | 2 | US20150322162 SEQ ID NO: 136 |
| APA97 | 2 | 2 | US20160053011 SEQ ID NO: 8 |
| APA98 | 2 | 2 | US20160053011 SEQ ID NO: 24 |
| APA99 | 2 | 2 | U.S Pat. No. 8,609,095 SEQ ID NO: 2; |
| | | | US20160089435 SEQ ID NO: 38 |
| APA100 | 2 | 2 | U.S Pat. No. 8,609,095 SEQ ID NO: 10 |
| APA101 | 2 | 2 | U.S Pat. No. 8,609,095 SEQ ID NO: 14; |
| | | | US20160089435 SEQ ID NO: 26 |
| APA102 | 2 | 2 | U.S Pat. No. 8,609,095 SEQ ID NO: 18 |
| APA103 | 2 | 2 | U.S Pat. No. 8,609,095 SEQ ID NO: 30 |
| APA104 | 2 | 2 | U.S Pat. No. 8,609,095 SEQ ID NO: 34 |
| APA105 | 2 | 2 | U.S Pat. No. 8,609,095 SEQ ID NO: 38 |
| APA106 | 2 | 2 | US20140356366 SEQ ID NO: 115; |
| | | | US20150104443 SEQ ID NO: 1 |
| APA107 | 1 | 10 | US20130089544 SEQ ID NO: 50 |
| APA108 | 1 | 10 | US20130089544 SEQ ID NO: 51 |
| APA109 | 1 | 10 | US20130089544 SEQ ID NO: 52 |
| APA110 | 1 | 10 | US20130089544 SEQ ID NO: 53 |
| APA111 | 1 | 10 | US20130266564 SEQ ID NO: 9 |
| APA112 | 1 | 10 | US20130266564 SEQ ID NO: 19 |
| APA113 | 1 | 10 | US20130266564 SEQ ID NO: 21 |
| APA114 | 1 | 10 | US20130266564 SEQ ID NO: 22 |
| APA115 | 1 | 10 | US20130266564 SEQ ID NO: 23 |
| APA116 | 1 | 10 | US20130266564 SEQ ID NO: 24 |
| APA117 | 1 | 10 | US20140010814 SEQ ID NO: 17 |
| APA118 | 2 | 10 | U.S Pat. No. 8,609,095 SEQ ID NO: 8; |
| | | | US20160089435 SEQ ID NO: 44 |
| APA119 | 2 | 10 | U.S Pat. No. 8,609,095 SEQ ID NO: 4; |
| | | | US20160089435 SEQ ID NO: 40 |
| APA120 | 2 | 10 | U.S Pat. No. 8,609,095 SEQ ID NO: 12 |
| APA121 | 2 | 10 | U.S Pat. No. 8,609,095 SEQ ID NO: 16; |
| | | | US20160089435 SEQ ID NO: 28 |
| APA122 | 2 | 10 | U.S Pat. No. 8,609,095 SEQ ID NO: 20 |
| APA123 | 2 | 10 | U.S Pat. No. 8,609,095 SEQ ID NO: 28; |
| | | | US20160089435 SEQ ID NO: 36 |
| APA124 | 2 | 10 | U.S Pat. No. 8,609,095 SEQ ID NO: 32 |
| APA125 | 2 | 10 | U.S Pat. No. 8,609,095 SEQ ID NO: 36 |
| APA126 | 2 | 10 | U.S Pat. No. 8,609,095 SEQ ID NO: 40 |
| APA127 | 1 | 11 | US20110129464 SEQ ID NO: 1 |
| APA128 | 1 | 11 | US20130089544 SEQ ID NO: 8 |
| APA129 | 1 | 11 | US20130089544 SEQ ID NO: 12 |
| APA130 | 1 | 11 | US20130089544 SEQ ID NO: 16 |
| APA131 | 1 | 11 | US20130089544 SEQ ID NO: 20 |
| APA132 | 1 | 11 | US20130089544 SEQ ID NO: 24 |
| APA133 | 1 | 11 | US20130089544 SEQ ID NO: 28, 32 |
| APA134 | 1 | 11 | US20130089544 SEQ ID NO: 36 |
| APA135 | 1 | 11 | US20130089544 SEQ ID NO: 40 |
| APA136 | 1 | 11 | US20130089544 SEQ ID NO: 44 |
| APA137 | 1 | 11 | US20130266564 SEQ ID NO: 7 |
| APA138 | 2 | 11 | US20090226455 SEQ ID NO: 1 |
| APA139 | 2 | 11 | US20080226659 SEQ ID NO: 4; |
| | | | US20100136033 SEQ ID NO: 4 |
| APA140 | 2 | 11 | US20080226659 SEQ ID NO: 5; |
| | | | US20100136033 SEQ ID NO: 5 |
| APA141 | 2 | 11 | US20130171148 SEQ ID NO: 5 |
| APA142 | 2 | 11 | US20130171148 SEQ ID NO: 12 |
| APA143 | 2 | 11 | US20130171148 SEQ ID NO: 19 |
| APA144 | 2 | 11 | US20130171148 SEQ ID NO: 26 |
| APA145 | 2 | 11 | US20130171148 SEQ ID NO: 33 |
| APA146 | 2 | 11 | US20130171148 SEQ ID NO: 40 |
| APA147 | 2 | 11 | US20130171148 SEQ ID NO: 44, |
| | | | 52, 54, 56 |
| APA148 | 2 | 11 | US20130171148 SEQ ID NO: 46 |
| APA149 | 2 | 11 | US20130171148 SEQ ID NO: 48, 50 |
| APA150 | 2 | 11 | US20130189271 SEQ ID NO: 5, |
| | | | 86, 88 |
| APA151 | 2 | 11 | US20130189271 SEQ ID NO: 12 |
| APA152 | 2 | 11 | US20130189271 SEQ ID NO: 19 |
| APA153 | 2 | 11 | US20130189271 SEQ ID NO: 26, 94 |
| APA154 | 2 | 11 | US20130189271 SEQ ID NO: 39 |
| APA155 | 2 | 11 | US20130189271 SEQ ID NO: 46 |
| APA156 | 2 | 11 | US20130189271 SEQ ID NO: 53 |
| APA157 | 2 | 11 | US20130189271 SEQ ID NO: 67 |
| APA158 | 2 | 11 | US20130189271 SEQ ID NO: 74 |
| APA159 | 2 | 11 | US20130189271 SEQ ID NO: 78, |
| | | | 80, 82 |
| APA160 | 2 | 11 | US20130189271 SEQ ID NO: 84 |
| APA161 | 2 | 11 | US20130189271 SEQ ID NO: 90 |
| APA162 | 2 | 11 | US20130189271 SEQ ID NO: 92 |
| APA163 | 2 | 11 | US20130189271 SEQ ID NO: 100, 102 |
| APA164 | 2 | 11 | US20130189271 SEQ ID NO: 104 |
| APA165 | 2 | 11 | US20130189271 SEQ ID NO: 106 |
| APA166 | 2 | 11 | US20130189271 SEQ ID NO: 32, 108 |
| APA167 | 2 | 11 | US20130189271 SEQ ID NO: 60, |
| | | | 110, 112 |
| APA168 | 2 | 11 | US20130189271 SEQ ID NO: 114 |
| APA169 | 2 | 11 | US20130189271 SEQ ID NO: 116 |
| APA170 | 2 | 11 | US20130189271 SEQ ID NO: 96, |
| | | | 98, 118 |
| APA171 | 2 | 11 | US20130189271 SEQ ID NO: 120 |
| APA172 | 2 | 11 | US20130189271 SEQ ID NO: 122, 126 |
| APA173 | 2 | 11 | US20130189271 SEQ ID NO: 124 |
| APA174 | 2 | 11 | US20140356366 SEQ ID NO: 117 |
| APA175 | 2 | 11 | US20150322162 SEQ ID NO: 137 |
| APA176 | 2 | 11 | US20150322162 SEQ ID NO: 138 |
| APA177 | 2 | 11 | US20150322162 SEQ ID NO: 139 |
| APA178 | 2 | 11 | US20150322162 SEQ ID NO: 140 |
| APA179 | 2 | 11 | US20150322162 SEQ ID NO: 141 |
| APA180 | 2 | 11 | US20160053011 SEQ ID NO: 10 |
| APA181 | 2 | 11 | US20160053011 SEQ ID NO: 26 |
| APA182 | 2 | 11 | US20160053011 SEQ ID NO: 247 |
| APA183 | 2 | 11 | US20160053011 SEQ ID NO: 249 |
| APA184 | 2 | 11 | US20160053011 SEQ ID NO: 251 |
| APA185 | 2 | 17 | US20100136033 SEQ ID NO: 6 |
| APA186 | 1 | 1 | US20100158926 SEQ ID NO: 2 |
| APA187 | 1 | 1 | US20100158926 SEQ ID NO: 6 |
| APA188 | 1 | 1 | US20100158926 SEQ ID NO: 10 |
| APA189 | 1 | 1 | US20100158926 SEQ ID NO: 14 |
| APA190 | 1 | 1 | US20100158926 SEQ ID NO: 26 |
| APA191 | 1 | 1 | US20100158926 SEQ ID NO: 30 |
| APA192 | 1 | 1 | US20100158926 SEQ ID NO: 42 |
| APA193 | 2 | 1 | U.S Pat. No. 9,562,110 SEQ ID NO: 3 |
| APA194 | 4 | 1 | U.S Pat. No. 8,907,069B2 SEQ ID NO: 7 |
| APA195 | 4 | 1 | U.S Pat. No. 8,907,069B2 SEQ ID NO: 8 |
| APA196 | 2 | 2 | U.S Pat. No. 9,150,663B2 SEQ ID NO: 16 |
| APA197 | 2 | 2 | U.S Pat. No. 9,150,663B2 SEQ ID NO: 18 |
| APA198 | 2 | 2 | U.S Pat. No. 9,150,663B2 SEQ ID NO: 20 |
| APA199 | 2 | 2 | U.S Pat. No. 9,150,663B2 SEQ ID NO: 22 |
| APA200 | 2 | 2 | U.S Pat. No. 9,155,802B2 SEQ ID NO: 26 |
| APA201 | 2 | 2 | U.S Pat. No. 9,527,913B2 SEQ ID NO: 6, 67 |
| APA202 | 2 | 2 | U.S Pat. No. 9,527,913B2 SEQ ID NO: 23 |
| APA203 | 2 | 2 | U.S Pat. No. 9,155,802B2 SEQ ID NO: 34 |
| APA204 | 2 | 2 | U.S Pat. No. 9,527,913B2 SEQ ID NO: 8, 69 |
| APA205 | 2 | 2 | U.S Pat. No. 9,527,913B2 SEQ ID NO: 27 |
| APA206 | 2 | 2 | U.S Pat. No. 9,155,802B2 SEQ ID NO: 42 |
| APA207 | 2 | 2 | U.S Pat. No. 9,527,913B2 SEQ ID NO: 48 |
| APA208 | 2 | 2 | U.S Pat. No. 9,527,913B2 SEQ ID NO: 52 |
| APA209 | 2 | 2 | U.S Pat. No. 9,527,913B2 SEQ ID NO: 50 |
| APA210 | 2 | 2 | US20130017598 SEQ ID NO: 17; |
| | | | US20110091372 SEQ ID NO: 98 |
| APA211 | 2 | 2 | US20130017598 SEQ ID NO: 19 |
| APA212 | 2 | 2 | US20130017598 SEQ ID NO: 25 |
| APA213 | 3 | 2 | U.S Pat. No. 9,150,663B2 SEQ ID NO: 26 |
| APA214 | 3 | 2 | U.S Pat. No. 9,150,663B2 SEQ ID NO: 24 |
| APA215 | 2 | 10 | U.S Pat. No. 9,155,802B2 SEQ ID NO: 28 |
| APA216 | 2 | 10 | U.S Pat. No. 9,527,913B2 SEQ ID NO: 25 |
| APA217 | 2 | 10 | U.S Pat. No. 9,155,802B2 SEQ ID NO: 36 |
| APA218 | 2 | 10 | U.S Pat. No. 9,527,913B2 SEQ ID NO: 29 |
| APA219 | 2 | 10 | U.S Pat. No. 9,155,802B2 SEQ ID NO: 44 |
| APA220 | 4 | 10 | U.S Pat. No. 8,907,069B2 SEQ ID NO: 9 |
| APA221 | 2 | 11 | U.S Pat. No. 9,150,663B2 SEQ ID NO: 17 |
| APA222 | 2 | 11 | U.S Pat. No. 9,150,663B2 SEQ ID NO: 19 |
| APA223 | 2 | 11 | U.S Pat. No. 9,150,663B2 SEQ ID NO: 21 |
| APA224 | 2 | 11 | U.S Pat. No. 9,150,663B2 SEQ ID NO: 23 |
| APA225 | 2 | 11 | U.S Pat. No. 9,527,913B2 SEQ ID NO: 7, 68 |
| APA226 | 2 | 11 | U.S Pat. No. 9,527,913B2 SEQ ID NO: 9, 70 |
| APA227 | 2 | 11 | U.S Pat. No. 9,527,913B2 SEQ ID NO: 49 |
| APA228 | 2 | 11 | U.S Pat. No. 9,527,913B2 SEQ ID NO: 53 |
| APA229 | 2 | 11 | U.S Pat. No. 9,527,913B2 SEQ ID NO: 51 |
| APA230 | 2 | 11 | US20130017598 SEQ ID NO: 16 |
| APA231 | 2 | 11 | US20130017598 SEQ ID NO: 18 |
| APA232 | 2 | 11 | US20130017598 SEQ ID NO: 24 |
| APA233 | 3 | 11 | U.S Pat. No. 915,066,3B2 SEQ ID NO: 27 |
| APA234 | 3 | 11 | U.S Pat. No. 915,066,3B2 SEQ ID NO: 25 |
| APA235 | 2 | 18 | WO2013156054; SEQ ID NO: 3 |
| APA236 | 5 | | US20140072582 SEQ ID NO: 34 |

In Table 28, the target number (Target No.) code is described in the following semi-colon delimited list where the target number is followed by the target (e.g., Target No. 1 with target ERBB2 is shown as Target No. 1-Target ERBB2). The targets represented by the codes in Table 28 include, but are not limited to, Target No. 1-Target ERBB2; Target No. 2-Target HER2; Target No. 3-Target HER2/CD3; Target No. 4-Target HER2/Dig; and Target No. 5-Target HER2/neu.

In Table 28, the description number (Description No.) code is described in the following semi-colon delimited list where the description number is followed by the description (e.g., Description No. 1 with description heavy chain is shown as Description No. 1-Description Heavy chain). The targets represented by the codes in Table 28 include, but are not limited to, Description No. 1-Description Heavy chain; Description No. 2-Description Heavy Chain—variable region; Description No. 3-Description Heavy chain 1; Description No. 4-Description Heavy chain constant, human IgG; Description No. 5-Description Heavy chain variable region-CDR1; Description No. 6-Description Heavy chain variable region-CDR2; Description No. 7-Description Heavy chain variable region-CDR3; Description No. 8-Description Herceptin Heavy chain variable region-CH1 (Heavy chain variable region(1-120)+CH1(121-218)); Description No. 9-Description H-GAMMA-1 (Heavy chain variable region(1-120)+CH1(121-218)+HINGE-REGION(219-233)+CH2(234-343)+CH3(344-450); Description No. 10-Description Light chain; Description No. 11-Description Light Chain—variable region; Description No. 12-Description Light chain (L-KAPPA (V-KAPPA(1-107)+C-KAPPA(108-214)); Description No. 13-Description Light chain 1, Anti-HER2; Description No. 14-Description Light chain variable region-CDR1 From U.S. Pat. No. 8,557,243; Description No. 15-Description Light chain variable region-CDR2 From U.S. Pat. No. 8,557,243; Description No. 16-Description Light chain variable region-CDR3 From U.S. Pat. No. 8,557,243; Description No. 17-Description Light chain, human subgroup; and Description No. 18-Description ScFv.

### AAV Particle Production

In some embodiments, disclosed herein is a method of making a genetic element. The method comprising providing a nucleic acid encoding a genetic element described herein and a backbone region suitable for replication of the genetic element in a cell, e.g., a bacterial cell (e.g., wherein the backbone region comprises one or both of a bacterial origin of replication and a selectable marker), and excising the genetic element from the backbone region, e.g., by cleaving the nucleic acid molecule at upstream and downstream of the genetic element. In some embodiments, the genetic element comprising a nucleic acid comprising a transgene encoding an antibody molecule (e.g., an antibody molecule that binds to HER2), will be incorporated into an AAV particle produced in a cell.

In some embodiments, disclosed herein is a method of making a recombinant AAV particle of the present disclosure, the method comprising (i) providing a host cell comprising a genetic element described herein and incubating the host cell under conditions suitable to enclose the genetic element in an AAV capsid polypeptide, e.g., an AAV capsid variant, e.g., a capsid polypeptide described herein (e.g., a capsid protein listed in Table 1 or 3-5), thereby making the recombinant AAV particle. In some embodiments, the method comprises prior to step (i), introducing a first nucleic acid comprising the genetic element into a cell. In some embodiments, the host cell comprises a second nucleic acid encoding the capsid protein. In some embodiments, the second nucleic acid is introduced into the host cell prior to, concurrently with, or after the first nucleic acid molecule.

Methods of making AAV particles are also described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in WO2015191508, the contents of which are herein incorporated by reference in their entirety.

In some embodiments, cells commonly used for production of a recombinant AAV particle include but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent publication No. 2002/0081721, and International Patent Publication Nos. WO 00/47757, WO 00/24916, and WO 96/17947, the contents of each of which are herein incorporated by reference in their entireties. In some embodiments, the AAV particles of the present disclosure may be produced in insect cells (e.g., Sf9 cells). In some embodiments, the AAV particles of the present disclosure may be produced in mammalian cells.

In some embodiments, the AAV particles of the present disclosure may be produced using triple transfection. In some embodiments, the AAV particles of the present disclosure may be produced by triple transfection in mammalian cells. In some embodiments, the AAV particles of the present disclosure may be produced by triple transfection in HEK293 cells.

The present disclosure provides a method for producing an AAV particle comprising the steps of: 1) co-transfecting competent bacterial cells with a bacmid vector and either a viral construct vector and/or AAV payload construct vector, e.g., a vector comprising a genetic element described herein, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, 5) harvesting and purifying the viral particle comprising a genetic element.

In some embodiments, the present disclosure provides a method for producing an AAV particle comprising the steps of 1) simultaneously co-transfecting mammalian cells, such as, but not limited to HEK293 cells, with a genetic element encoding an antibody molecule, a construct expressing rep and cap genes and a helper construct, 2) harvesting and purifying the AAV particle comprising the genetic element.

In some embodiments, the viral construct vector(s) used for AAV production may contain a nucleotide sequence encoding the AAV capsid proteins where the initiation codon of the AAV VP1 capsid protein is a non-ATG, e.g., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In some embodiments, the viral construct vector(s) used for AAV production may contain a nucleotide sequence encoding the AAV rep proteins where the initiation codon of the AAV rep protein or proteins is a non-ATG. In some embodiments, a single coding sequence is used for the Rep78 and Rep52 proteins, wherein initiation codon for translation of the Rep78 protein is a suboptimal initiation codon, selected from the group consisting of ACG, TTG, CTG, and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the genetic element of the AAV particle optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are selected from those described in International Application No. WO 96/23810; Heim et al., Current Biology 2:178-182 (1996); Heim et al., Proc. Natl. Acad. Sci. USA (1995); or Heim et al., Science 373:663-664 (1995); WO 96/30540, the contents of each of which are incorporated herein by reference in their entireties).

The genetic elements encoding an anti-HER2 antibody molecule payload described herein may be useful in the fields of human disease, veterinary applications and a variety of in vivo and in vitro settings. The AAV particles of the present disclosure may be useful in the field of medicine for the treatment, prophylaxis, palliation or amelioration of diseases and/or disorders caused by HER2 over-expression. In some embodiments, the AAV particles are used for the prevention and/or treatment of a disease and/or disorder associated with over-expression of HER2. In some embodiments, the disease and/or disorder associated with HER2 over-expression includes, but is not limited to tumors, cancers, neoplastic tissue, pre-malignant and non-neoplastic or non-malignant hyperproliferative disorders. In some embodiments, the disease associated with HER2 over-expression is cancer. In some embodiments, the cancer is metastatic breast cancer (e.g., brain metastases).

The present disclosure also provides in some embodiments, a pharmaceutical composition comprising an AAV particle described herein, e.g., an AAV particle comprising a genetic element encoding an anti-HER2 antibody molecule, and a pharmaceutically acceptable excipient. The present disclosure also provides in some embodiments, a method of treating a subject in need thereof comprising administering to the subject a therapeutically effective amount of the pharmaceutical composition described herein.

In some embodiments, the pharmaceutical composition is administered to a subject intravenously, intracerebroventricularly, via intraparenchymal administration, intrathecally, via subpial administration, intramuscularly, or a combination thereof. Certain embodiments of the method provide that the subject is treated for a disease and/or disorder associated with over expression of HER2. In one aspect of the method, a symptom, e.g., a pathological feature, of the neurological disorder and/or a disease associated with over expression of HER2 is reduced, alleviated, and/or the progression of the disease and/or disorder associated with over expression of HER2 is halted, slowed, ameliorated, or reversed.

Also described herein are compositions, methods, processes, kits and devices for the design, preparation, manufacture and/or formulation of an AAV particle, e.g., an AAV particle described herein for the vectorized delivery of an antibody molecule. In some embodiments, a payload, such as but not limited to anti-HER2 antibody molecule, may be encoded by a nucleic acid comprising or contained within plasmids or vectors or recombinant adeno-associated viruses (AAVs).

The present disclosure also provides administration and/or delivery methods for encoded payloads (e.g., antibody molecules), genetic elements, AAV vectors, and viral particles, e.g., AAV particles, for the treatment and/or amelioration of diseases and/or disorders associated with over expression of HER2 (e.g., cancer).

### Cells

In some embodiments, disclosed herein is a cell comprising a genetic element and/or an AAV vector of the present disclosure.

Viral production disclosed herein describes processes and methods for producing an AAV particle that contacts a cell to deliver a payload, e.g. a genetic element comprising a transgene encoding a payload, e.g., an antibody molecule described herein.

In some embodiments, an AAV particle described herein may be produced in a cell, e.g., a viral replication cell, that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which is herein incorporated by reference in its entirety. The cell, e.g., viral replication cell, may be selected from any biological organism, including a prokaryotic (e.g., bacterial) cell, and a eukaryotic cell, including for example, insect cells, yeast cells and mammalian cells. Viral replication cells may comprise mammalian cells such as A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. In some embodiments, a viral replication cell comprises a cell derived from a mammalian species including, but not limited to, a human, a monkey, a mouse, a rat, a rabbit, and a hamster or a cell type, including but not limited to a fibroblast, a hepatocyte, a tumor cell, a cell line transformed cell, etc.

### Production of AAV Particles

In some embodiments, production of an AAV particle disclosed herein describes processes and methods for producing an AAV particle that contacts a cell, e.g., a target cell, to deliver a payload, e.g. an antibody molecule and/or a genetic element comprising a nucleotide sequence comprising a transgene encoding a payload, e.g., an antibody molecule.

In some embodiments, an AAV particle of the present disclosure may be produced in a cell, e.g., a viral replication cell that comprises a mammalian cell.

In some embodiments, a viral replication cell for production of a recombinant AAV particle described herein includes, but is not limited to 293 cells, COS cells. HeLa cells, KB cells, and other mammalian cell lines as described in U.S. Pat. Nos. 6,156,303, 5,387,484, 5,741,683, 5,691,176, and 5,688,676; U.S. patent application 2002/0081721, and International Patent Applications WO 00/47757, WO 00/24916, and WO 96/17947, the contents of each of which are herein incorporated by reference in their entireties.

In some embodiments, an AAV particle is produced in a mammalian-cell wherein all three VP proteins are expressed at a stoichiometry approaching 1:1:10 (VP1:VP2:VP3). In some embodiments, the regulatory mechanisms that allow this controlled level of expression include the production of two mRNAs, one for VP1, and the other for VP2 and VP3, produced by differential splicing.

In other embodiments, an AAV particles is produced in a mammalian cell using a triple transfection method wherein a genetic element comprising a nucleotide sequence comprising a transgene encoding a payload (e.g., an antibody molecule described herein), parvoviral Rep and parvoviral Cap and a helper construct are comprised within three different constructs. In some embodiments, the triple transfection method of the three components of AAV particle production is utilized to produce small lots of virus for assays including transduction efficiency, target tissue (tropism) evaluation, and stability.

### Baculovirus

In some embodiments, the production of an AAV particle disclosed herein comprises processes and methods for producing an AAV particle that contacts a cell, e.g., a target cell, to deliver a genetic element which comprises a nucleotide sequence comprising a transgene encoding a payload (e.g., a an antibody molecule described herein).

Briefly, a viral construct vector (e.g., encoding the structural, non-structural, components of the particle) and a genetic element each incorporated by a transposon donor/acceptor system into a bacmid, also known as a baculovirus plasmid, by standard molecular biology techniques known and performed by a person skilled in the art. Transfection of a separate cell, e.g., a viral replication cell, or population of cells produces two baculoviruses, one that comprises the viral construct expression vector, and another that comprises the genetic element. The two baculoviruses may be used to infect a single viral replication cell population for production of AAV particles.

In some embodiments, a baculovirus expression vector for producing viral particles in an insect cell, including but not limited to a Spodoptera frugiperda (Sf9) cell, provides high titers of viral particle product, e.g., an AAV particle described herein. In some embodiments, a recombinant baculovirus encoding the viral construct expression vector and genetic element encoding a payload initiates an infection, e.g., a productive infection of a cell, e.g., a viral replicating cell, or population of cells. In some embodiments, an infectious baculovirus particle released from the primary infection secondarily infect at an additional cell or plurality of cells in the culture, exponentially infecting the entire cell culture population in a number of infection cycles that is a function of the initial administration of infection, see Urabe, M. et al., J Virol. 2006 February; 80 (4):1874-85, the contents of which are herein incorporated by reference in their entirety.

In some embodiments, the production system utilizes a titerless infected-cells preservation and scale-up system. Small scale seed cultures of cells, e.g., viral producing cells are transfected with viral expression constructs encoding the structural, non-structural, components of the viral particle, e.g., an AAV particle described herein. Baculovirus-infected viral producing cells are harvested into aliquots that may be cryopreserved in liquid nitrogen; the aliquots retain viability and infectivity for infection of large scale viral producing cell culture Wasilko D J et al., Protein Expr Purif. 2009 June; 65(2):122-32, the contents of which are herein incorporated by reference in their entirety.

In some embodiments, a genetically stable baculovirus may be used as a source of the one or more of the components for producing an AAV particle of the present disclosure in invertebrate cells. In some embodiments, a defective baculovirus expression vector may be maintained episomally in insect cells. Tn some embodiments, the bacmid vector is engineered with replication control elements, including but not limited to promoters, enhancers, and/or cell-cycle regulated replication elements.

In some embodiments, a baculoviruses may be engineered with a (non-) selectable marker for recombination into the chitinase/cathepsin locus. In some embodiments, the chia/v-cath locus is non-essential for propagating baculovirus in tissue culture, and the V-cath (EC 3.4.22.50) is a cysteine endoprotease that is most active on Arg-Arg dipeptide containing substrates. In some embodiments, the Arg-Arg dipeptide is present in densovirus and parvovirus capsid structural proteins but infrequently occurs in dependovirus VP1.

In some embodiments, stable cells, e.g., viral replication cells, permissive for baculovirus infection are engineered with at least one stable integrated copy of any of the elements necessary for AAV replication and particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, any of the genetic elements, e.g., any of BA_ITR1-BA_ITR9, e.g., as described in Tables 6, 17-19, 23-26, 40, 42, 43, 68, and 69, is introduced into a bacmid. The bacmid may further comprise replication and transcription control elements, including but not limited to promoters, enhancers, and/or cell-cycle regulated replication elements, as well as antibiotic or other selection markers to permit cloning and/or maintenance of infectious baculovirus DNA in a suitable bacterial host, such as E. coli.

In some embodiments, AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle, e.g., AAV particle, production scale typically comprise increasing the number of viral replication cells. In some embodiments, a viral replication cell comprises an adherent cell. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, NY) and NUNC™ CELL FACTORY™ (Thermo Scientific, Waltham, MA.) In some cases, large-scale adherent cell surfaces may comprise from about 1,000 cm² to about 100,000 cm². In some cases, large-scale adherent cell cultures may comprise from about 10⁷ to about 10⁹ cells, from about 10⁸ to about 10¹⁰ cells, from about 10⁹ to about 10¹² cells or at least 10¹² cells. In some cases, large-scale adherent cultures may produce from about 10 to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds [e.g. polyethylencimine (PEI)] or the use of non-chemical methods (e.g. electroporation.) With cells grown in suspension, transfection methods may include, but are not limited to the use of calcium phosphate and the use of PEI. In some cases, transfection of large scale suspension cultures may be carried out according to the section entitled “Transfection Procedure” described in Feng, L. et al., 2008. Biotechnol Appl. Biochem. 50:121-32, the contents of which are herein incorporated by reference in their entirety. In some embodiments, PEI-DNA complexes may be formed for introduction of plasmids to be transfected. In some embodiments, a cell being transfected with PEI-DNA complexes may be ‘shocked’ prior to transfection, which comprises lowering cell culture temperatures to 4° C. for a period of about 1 hour. In some embodiments, a cell culture may be shocked for a period of from about 10 minutes to about 5 hours. In some embodiments, a cell culture may be shocked at a temperature of from about 0° C. to about 20° C.

In some embodiments, a transfection may include a vector for expression of an RNA effector molecule to reduce expression of nucleic acids from a genetic element and/or AAV vector. In some embodiments, such a method may enhance the production of viral particles by reducing cellular resources wasted on expressing a genetic element encoding a payload. In some embodiments, such a method may be carried according to those taught in US Publication No. US2014/0099666, the contents of which are herein incorporated by reference in their entirety.

### III. FORMULATION AND DELIVERY

### Pharmaceutical Compositions

According to the present disclosure, the AAV particle described herein may be prepared as a pharmaceutical composition. In some embodiments, the pharmaceutical composition comprises at least one active ingredients. In some embodiments, the pharmaceutical composition comprises a pharmaceutically acceptable excipient.

In some embodiments, the relative amount of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the pharmaceutical composition comprising an AAV particle described herein may comprise a genetic element encoding a payload, e.g., an antibody molecule. As a non-limiting example, the pharmaceutical composition may contain an AAV particle with at least 1, 2, 3, 4, 5, or more genetic elements encoding an antibody molecule. In some embodiments, the pharmaceutical composition comprises a nucleic acid encoding a transgene encoding a payload comprising an antibody molecule.

The present disclosure also provides in some embodiments, a pharmaceutical composition suitable for administration to a human and/or any other animal, e.g., to non-human animals, e.g. non-human mammals. The present disclosure also provides modification of a pharmaceutical composition suitable for administration to a human such that it is also suitable for administration to various animals. In some embodiments, a subject to which administration of the pharmaceutical compositions is contemplated include, but are not limited to, a human or a non-human primate; and/or a mammal, including cattle, pigs, horses, sheep, cats, dogs, mice, rats, birds, e.g., such as poultry, chickens, ducks, geese, and/or turkeys.

In some embodiments, the pharmaceutical composition described herein is administered to a human, e.g., a human patient, or a human subject.

### Formulations

In some embodiments, the AAV particle can be formulated using an excipient to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload.

Formulations of the present disclosure can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. In some embodiments, a pharmaceutical composition comprises an AAV particle described herein and at least one active ingredient and optionally a pharmaceutically acceptable excipient.

In general, such preparatory methods include the step of associating the active ingredient with an excipient and/or one or more other accessory ingredients. In some embodiments, an active ingredient can comprise an AAV particle carrying a payload region encoding the polypeptides or to the antibody or antibody-based composition encoded by a genetic element of by an AAV particle as described herein.

A pharmaceutical composition in accordance with the present disclosure may be prepared, packaged, and/or sold in bulk, as a single unit dose, and/or as a plurality of single unit doses. In some embodiments, a unit dose is a discrete amount of the pharmaceutical composition comprising a predetermined amount of the active ingredient. In some embodiments, the amount of the active ingredient is generally equal to the dosage of the active ingredient which would be administered to a subject and/or a convenient fraction of such a dosage such as, for example, one-half or one-third of such a dosage.

In some embodiments, an AAV particle may be formulated in phosphate buffered saline (PBS), in combination with an ethylene oxide/propylene oxide copolymer (also known as Pluronic or poloxamer). In some embodiments, an AAV particle may be formulated in PBS with 0.001% Pluronic acid (F-68) (poloxamer 188) at a pH of about 7.0. In some embodiments, an AAV particle may be formulated in PBS with 0.001% Pluronic acid (F-68) (poloxamer 188) at a pH of about 7.3. In some embodiments, an AAV particle may be formulated in PBS with 0.001% Pluronic acid (F-68) (poloxamer 188) at a pH of about 7.4. In some embodiments, an AAV particle may be formulated in a solution comprising sodium chloride, sodium phosphate and an ethylene oxide/propylene oxide copolymer. In some embodiments, an AAV particle may be formulated in a solution comprising sodium chloride, sodium phosphate dibasic, sodium phosphate monobasic and poloxamer 188/Pluronic acid (F-68).

In some embodiments, an AAV particle may be formulated in a solution comprising about 180 mM sodium chloride, about 10 mM sodium phosphate and about 0.001% poloxamer 188. In some embodiments, this formulation may be at a pH of about 7.3. In some embodiments, the concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate in the final solution may be 1 mM-50 mM. As non-limiting examples, the concentration of sodium phosphate in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. In some embodiments, the concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, an AAV particle described herein may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. In some embodiments, the concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, an AAV particle described herein may be administered with a pharmaceutically acceptable salt. Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. Lists of additional suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Relative amounts of an active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

In some embodiments, a formulation comprising an AAV particle described herein comprises a sufficient, e.g., effective amount, of AAV particles for expression of an antibody molecule, e.g., an antibody molecule that binds to HER2. In some embodiments, an AAV particle comprises a genetic element encoding at least 1, 2, 3, 4, or 5 antibody molecules.

The present disclosure provides, in some embodiments, an AAV particle formulated for delivery to the central nervous system (CNS). In some embodiments, an agent that crosses the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

### Excipients and Diluents

In some embodiments, a pharmaceutically acceptable excipient may be substantially pure, e.g., having at least 90%, at least 95%, at least 96%, at least 97%, at least 98%, at least 99%, or 100% purity. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

In some embodiments, an excipient, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety).

In some embodiments, a pharmaceutically acceptable excipient may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, a pharmaceutically acceptable solvent may include, for example, ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, and benzyl benzoate.

In some embodiments, the exemplary diluent includes, but is not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, a formulation comprising an AAV particle described herein may comprise an inactive ingredient. In some embodiments, an inactive ingredient comprises an agent that does not contribute to the activity of the active ingredient of the pharmaceutical composition included in formulations.

In some embodiments, a formulation of a pharmaceutical composition comprising an AAV particle disclosed herein may include cations or anions. In some embodiments, the formulation includes a metal cation such as, but not limited to, Zn2+, Ca2+, Cu2+, Mn2+, Mg+ and combinations thereof. As a non-limiting example, a formulation may include a polymer and a complex with a metal cation (See e.g., U.S. Pat. Nos. 6,265,389 and 6,555,525, each of which is herein incorporated by reference in its entirety).

### IV. ADMINISTRATION AND DOSING

### Administration

In some embodiments, an AAV particle of the present disclosure may be administered by any delivery route which results in a therapeutically effective outcome. In some embodiments, the delivery route includes, but is not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

A further aspect of the present invention relates to a method of treating a subject having or diagnosed with having cancer expressing HER2/neu, comprising administering to the subject an effective amount of a pharmaceutical composition comprising an AAV particle or vector, genetic element, or isolated nucleic acid as described herein. In some embodiments, the subject is administered an AAV particle of the present invention. In some embodiments, the subject is being treated for a disease associated with HER2/neu expression. In some embodiments, the disease associated with HER2/neu expression is a HER2/neu-positive solid tumor. In some embodiments, the HER2/neu positive tumor is metastatic. In some embodiments the HER/neu positive cancer is breast cancer, gastric cancer, gastroesophageal junction cancer, colorectal cancer, lung cancer (e.g., non-small cell lung carcinoma), pancreatic cancer, bladder cancer, salivary duct cancer, ovarian cancer (e.g., epithelial ovarian cancer), endometrial cancer, prostate cancer, bone cancer and brain cancer. In some embodiments, the HER2/neu positive cancer has metastasized to the central nervous system (CNS). In some embodiments, the HER2/neu positive cancer is breast cancer. In some embodiments, the subject is a human. In some embodiments, the subject has previously undergone surgical resection of a HER2/neu-positive solid tumor. In some embodiments, the AAV particle is administered to the subject intramuscularly, intravenously, intratumorally, intracerebrally, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM). In some embodiments, the AAV particle is administered to the subject intravenously. In some embodiments, the AAV particle is administered to the subject intratumorally. In some embodiments, the AAV particle is administered to the subject via intra-cisterna magna injection (ICM). In some embodiments, the AAV particle is administered prior to, concurrently with, or post a surgical resection of a HER2/neu-positive solid tumor. In some embodiments, the AAV particle is administered to a site of surgical resection of a HER2/neu-positive solid tumor in the subject. In some embodiments, the AAV particle is administered to the area around a site of surgical resection of a HER2/neu-positive solid tumor, e.g., the margins of the tumor, in the subject.

In some embodiments, an AAV particle described herein may be administered to a subject via such a route that it is able to cross the blood-brain barrier, vascular barrier, or other epithelial barrier. In some embodiments, an AAV particle of the present disclosure may be administered in any suitable form, either as a liquid solution or suspension, as a solid form suitable for liquid solution or suspension in a liquid solution. In some embodiments, an AAV particle may be formulated with any appropriate and pharmaceutically acceptable excipient.

In some embodiments, an AAV particle of the present disclosure may be delivered to a subject via a single route administration. In some embodiments, an AAV particle of the present disclosure may be delivered to a subject via a multi-site route of administration. In some embodiments, a subject may be administered at 2, 3, 4, 5, or more than 5 sites.

In some embodiments, an AAV particle of the present disclosure is administered via a bolus infusion. In some embodiments, an AAV particle of the present disclosure is administered via sustained delivery over a period of minutes, hours, or days. In some embodiments, the infusion rate may be changed depending on the subject, distribution, formulation, and/or another delivery parameter. In some embodiments, an AAV particle of the present disclosure is administered using a controlled release, e.g., a release profile that conforms to a particular pattern of release to effect a therapeutic outcome. In some embodiments, an AAV particle of the present disclosure is administered using a sustained release, e.g., a release profile that conforms to a release rate over a specific period of time.

In some embodiments, an AAV particle may be delivered by more than one route of administration. As non-limiting examples of combination administrations, an AAV particle may be delivered by intrathecal and intracerebroventricular, or by intravenous and intraparenchymal administration. In some embodiments, an AAV particle may be delivered intra-tumoral and/or delivered into resection void (e.g., the space left from the removal of a tumor).

### Intravenous Administration

In some embodiments, an AAV particle described herein may be administered to a subject by systemic administration. In some embodiments, the systemic administration is intravenous administration. In another embodiment, the systemic administration is intraarterial administration. In some embodiments, an AAV particle of the present disclosure may be administered to a subject by intravenous administration. In some embodiments, the intravenous administration may be achieved by subcutaneous delivery. In some embodiments, the AAV particle is administered to the subject via focused ultrasound (FUS), e.g., coupled with the intravenous administration of microbubbles (FUS-MB) or MRI-guided FUS coupled with intravenous administration, e.g., as described in Terstappen et al. (Nat Rev Drug Discovery, https://doi.org/10.1038/s41573-021-00139-y (2021)), the contents of which are incorporated herein by reference in its entirety. In some embodiments, the AAV particle is administered to the subject intravenously. In some embodiments, the intravenous administration may be achieved by a tail vein injection (e.g., in a mouse model). In some embodiments, the intravenous administration may be achieved by retro-orbital injection.

### Administration to the CNS

In some embodiments, an AAV particle described herein may be delivered by direct injection into the brain. As a non-limiting example, the brain delivery may be by intrahippocampal administration. In some embodiments, an AAV particle of the present disclosure may be administered to a subject by intraparenchymal administration. In some embodiments, the intraparenchymal administration is to tissue of the central nervous system. In some embodiments, an AAV particle of the present disclosure may be administered to a subject by intracranial delivery (See, e.g., U.S. Pat. No. 8,119,611; the content of which is incorporated herein by reference in its entirety). In some embodiments, an AAV particle described herein may be delivered by injection into the CSF pathway. Non-limiting examples of delivery to the CSF pathway include intrathecal and intracerebroventricular administration. In some embodiments, the AAV particle is administered via intra cisterna magna (ICM).

In some embodiments, an AAV particle may be delivered to the brain by systemic delivery. As a non-limiting example, the systemic delivery may be by intravascular administration. As a non-limiting example, the systemic or intravascular administration may be intravenous.

In some embodiments, an AAV particle of the present disclosure may be delivered by an intraocular delivery route. A non-limiting example of an intraocular administration includes an intravitreal injection.

### Intramuscular Administration

In some embodiments, an AAV particle described herein may be delivered by intramuscular administration. Without wishing to be bound by theory, it is believed in some embodiments, that the multi-nucleated nature of muscle cells provides an advantage to gene transduction subsequent to AAV delivery. In some embodiments, cells of the muscle are capable of expressing recombinant proteins with the appropriate post-translational modifications. Without wishing to be bound by theory, it is believed in some embodiments, the enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered. In some embodiments, an AAV particle of the present disclosure may be delivered by an intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, an AAV particle of the present disclosure is administered to a subject and transduces the muscle of a subject. As a non-limiting example, an AAV particle is administered by intramuscular administration. In some embodiments, an AAV particle of the present disclosure may be administered to a subject by subcutaneous administration. In some embodiments, the intramuscular administration is via systemic delivery. In some embodiments, the intramuscular administration is via intravenous delivery. In some embodiments, the intramuscular administration is via direct injection to the muscle.

In some embodiments, the muscle is transduced by administration, e.g., intramuscular administration. In some embodiments, an intramuscular delivery comprises administration at one site. In some embodiments, an intramuscular delivery comprises administration at more than one site. In some embodiments, an intramuscular delivery comprises administration at two sites. In some embodiments, an intramuscular delivery comprises administration at three sites. In some embodiments, an intramuscular delivery comprises administration at four sites. In some embodiments, an intramuscular delivery comprises administration at more than four sites. In some embodiments, intramuscular delivery is combined with at least one other method of administration.

In some embodiments, an AAV particle may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, an AAV particle of the present disclosure may be administered to a subject by intraparenchymal administration. In some embodiments, the intraparenchymal administration is to muscle tissue. In some embodiments, an AAV particle of the present disclosure is delivered as described in Bright et al 2015 (Neurobiol Aging. 36(2):693-709), the contents of which are herein incorporated by reference in their entirety. In some embodiments, an AAV particle of the present disclosure is administered to the gastrocnemius muscle of a subject. In some embodiments, an AAV particle of the present disclosure is administered to the bicep femorii of the subject. In some embodiments, an AAV particles of the present disclosure is administered to the tibialis anterior muscles. In some embodiments, an AAV particle of the present disclosure is administered to the soleus muscle.

### Depot Administration

As described herein, in some embodiments, a pharmaceutical composition and/or an AAV particle of the present disclosure are formulated in depots for extended release. Generally, specific organs or tissues are targeted for administration.

In some embodiments, a pharmaceutical composition and/or an AAV particle of the present disclosure are spatially retained within or proximal to target tissues. Provided are methods of providing a pharmaceutical composition, an AAV particle, to target tissues of mammalian subjects by contacting target tissues (which comprise one or more target cells) with the pharmaceutical composition and/or the AAV particle, under conditions such that they are substantially retained in target tissues, e.g., such that at least 10, 20, 30, 40, 50, 60, 70, 80, 85, 90, 95, 96, 97, 98, 99, 99.9, 99.99 or greater than 99.99% of the composition is retained in the target tissues. In some embodiments, retention is determined by measuring the amount of pharmaceutical composition and/or AAV particle, that enter a target cell or a plurality of target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of a pharmaceutical composition and/or an AAV particle, administered to a subject are present intracellularly at a period of time following administration. For example, intramuscular injection to a subject may be performed using aqueous compositions comprising a pharmaceutical composition and/or an AAV particle of the present disclosure and a transfection reagent, and retention is determined by measuring the amount of the pharmaceutical composition and/or the AAV particle, present in the muscle cell or plurality of muscle cells.

In some embodiments, disclosed herein are methods of providing a pharmaceutical composition and/or an AAV particle of the present disclosure to a tissue of a subject, by contacting the tissue (comprising a cell, e.g., a plurality of cells) with the pharmaceutical composition and/or the AAV particle under conditions such that they are substantially retained in the tissue. In some embodiments, a pharmaceutical composition and/or AAV particle described herein comprise a sufficient amount of an active ingredient such that the effect of interest is produced in at least one cell. In some embodiments, a pharmaceutical composition and/or an AAV particle generally comprise one or more cell penetration agents. In some embodiments, the disclosure provides a naked formulations (such as without cell penetration agents or other agents), with or without pharmaceutically acceptable carriers.

### Delivery

In some embodiments, an AAV particle or a pharmaceutical composition of the present disclosure may be administered or delivered using the methods for treatment of disease described in U.S. Pat. No. 8,999,948, or International Publication No. WO2014178863, the contents of which are herein incorporated by reference in their entirety.

In some embodiments, an AAV particle or a pharmaceutical composition of the present disclosure may be administered or delivered using the methods for delivering gene therapy as described in US Application No. 20150126590, the contents of which are herein incorporated by reference in their entirety.

In some embodiments, an AAV particle or a pharmaceutical composition of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, an AAV particle or a pharmaceutical composition of the present disclosure may be administered or delivered using the methods for delivering a protein using an AAV vector described in European Patent Application No. EP2678433, the contents of which are herein incorporated by reference in their entirety.

In some embodiments, an AAV particle or a pharmaceutical composition of the present disclosure may be administered or delivered using the methods for delivering DNA to the bloodstream described in U.S. Pat. No. 6,211,163, the contents of which are herein incorporated by reference in their entirety.

In some embodiments, an AAV particle or a pharmaceutical composition of the present disclosure may be administered or delivered using the methods for delivering a payload to the central nervous system described in U.S. Pat. No. 7,588,757, the contents of which are herein incorporated by reference in their entirety.

In some embodiments, an AAV particle or a pharmaceutical composition of the present disclosure may be administered or delivered using the methods for delivering a payload described in U.S. Pat. No. 8,283,151, the contents of which are herein incorporated by reference in their entirety.

In some embodiments, an AAV particle or a pharmaceutical composition of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, an AAV particle or a pharmaceutical composition of the present disclosure may be administered or delivered using the methods for delivering a payload to neural cells described in International Patent Publication No. WO2012057363, the contents of which are herein incorporated by reference in their entirety.

### Delivery to Cells

The present disclosure also provides a method of delivering to a cell or a tissue any of the above-described AAV particles, comprising contacting the cell or tissue with said AAV particle or contacting the cell or tissue with a formulation comprising said AAV particle, or contacting the cell or tissue with any of the described compositions, e.g., a pharmaceutical composition described herein. The method of delivering an AAV particle to a cell or tissue can be accomplished in vitro, ex vivo, or in vivo.

### Delivery to Subjects

The present disclosure also provides a method of delivering to a subject, e.g., a mammalian subject, any of the above-described AAV particles comprising administering to the subject said AAV particle, or administering to the subject a formulation comprising said AAV particle, or administering to the subject any of the described compositions, including pharmaceutical compositions.

### Dose and Regimen

The present disclosure provides methods of administering an AAV particle described herein to a subject in need thereof. A pharmaceutical, diagnostic, or prophylactic AAV particle and composition of the present disclosure may be administered to a subject using any amount and any route of administration effective for preventing, treating, managing, or diagnosing a disease, disorder and/or condition. In some embodiments, an effective amount of an AAV particle will vary from subject to subject, depending on the species, age, and general condition of the subject, the severity of the disease, the particular composition, its mode of administration, its mode of activity, and the like. The subject may be a human, a mouse, a non-human primate, a mammal, or an animal. In some embodiments, a composition in accordance with the disclosure can be formulated in unit dosage form for ease of administration and uniformity of dosage. In some embodiments, the total daily usage of a composition of the present disclosure may be decided by the attending physician within the scope of sound medical judgment. In some embodiments, a therapeutically effective, prophylactically effective, or appropriate diagnostic dose level for a subject may depend upon a variety of factors including the disorder being treated and the severity of the disorder; the activity of the specific payload employed; the specific composition employed; the age, body weight, general health, sex and diet of the patient; the time of administration, route of administration, and rate of excretion of the AAV particle employed; the duration of the treatment; drugs used in combination or coincidental with the AAV particle employed; and like factors well known in the medical arts.

In some embodiments, a pharmaceutical composition comprising an AAV particle of the present disclosure may be administered at dosage levels sufficient to deliver from about 0.0001 mg/kg to about 100 mg/kg, e.g., from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. In some embodiments, the above dosing concentrations may be converted to vg or viral genomes (e.g., a genetic element as described herein) per kg or into total viral genomes administered by one of skill in the art.

In some embodiments, a pharmaceutical composition comprising an AAV particle in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, AAV particles may be administered at 50 μl/site and/or 150 μl/site.

In some embodiments, a dosage may be delivered using multiple administrations (e.g., two, three, four, or more than four administrations). In some embodiments, when multiple administrations are used, a split dosing regimen, such as those described herein, may be used. In some embodiments, a split dose is the division of single unit dose or total daily dose into two or more doses, e.g., two or more administrations of the single unit dose. In some embodiments, a single unit dose is a dose of any therapeutic administered in one dose/at one time/single route/single point of contact, e.g., a single administration event.

In some embodiments, a dosage of an AAV particle of the present disclosure may be administered as a pulse dose or as a continuous flow. In some embodiments, a pulse dose comprises a series of single unit doses of any therapeutic administered with a set frequency over a period of time. In some embodiments, a continuous flow comprises a dose of therapeutic administered continuously for a period of time in a single route/single point of contact, e.g., a continuous administration event. In some embodiments, a total daily dose, e.g., an amount given or prescribed in 24-hour period, may be administered by any of these methods, or as a combination of these methods, or by any other methods suitable for a pharmaceutical administration.

In some embodiments, delivery of an AAV particle of the present disclosure to a subject provides neutralizing activity to a subject. In some embodiments, the neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, delivery of an AAV particle may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10⁵, 4×10⁵, 5×10⁵, 6×10⁵, 7×10⁵, 8×10⁵, 9×10⁵, or 1×10¹⁶ VG. As a non-limiting example, the total dose is 1×10¹³ VG. As another non-limiting example, the total dose is 2.1×10¹² VG.

In some embodiments, delivery of an AAV particle may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹, 2×10¹⁵, 3×10¹⁵, 4×10¹⁵, 5×10¹⁵, 6×10¹⁵, 7×10⁵, 8×10¹⁵, 9×10¹⁵, or 1×10¹⁶ VG/mL. In some embodiments, the delivery comprises a composition concentration of 1×10¹³ VG/mL. In some embodiments, the delivery comprises a composition concentration of 2.1×10¹² VG/mL.

### Combinations

In some embodiments, an AAV particle described herein may be used in combination with one or more other therapeutic, prophylactic, research or diagnostic agents. In some embodiments, the agents are administered at the same time and/or are formulated for delivery together, e.g., simultaneous delivery. In some embodiments, the agents are formulated for sequential delivery. In some embodiments, compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### Measurement of Expression

In some embodiments, the methods provided herein further comprise evaluating, e.g., measuring, the level of a payload, e.g., an antibody molecule, generated in a subject, e.g., in a cell or tissue of the subject. In some embodiments, the cell is a neuronal cell. In some embodiments, the cell is an endothelial cell. In some embodiments, the tissue is a central nervous system (CNS) tissue, e.g., a brain tissue. In some embodiments, the tissue is one known to show increased levels of an ErbB receptor tyrosine kinase. The ErbB receptor tyrosine kinase family includes at least four distinct members including Epidermal Growth Factor Receptor (EGFR or ErbB1), HER2 (ErbB2 or p185neu), HER3 (ErbB3) and HER4 (ErbB4 or tyro2). Amplification of HER2 is often observed in breast and ovarian cancers. Overexpression of HER2 (frequently but not uniformly due to gene amplification) has also been observed in carcinomas of the stomach, endometrium, salivary gland, lung, kidney, colon, thyroid, pancreas and bladder. In some embodiments, the tissue is a breast or ovarian tissue. In some embodiments, the tissue is a stomach tissue, endometrium tissue, salivary gland tissue, lung tissue, kidney tissue, colon tissue, thyroid tissue, pancreas tissue or bladder tissue. In some embodiments, the method further comprises a blood test, an imaging test, a CNS biopsy sample, or an aqueous cerebral spinal fluid biopsy. In some embodiments, measuring the level of antibody molecules is performed prior to, during, or subsequent to treatment with the AAV particle, e.g., plurality of AAV particles.

In some embodiments, expression of a payload encoded by a genetic element described herein may be measured using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, immunohistochemistry, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

### V. METHODS AND USES OF THE COMPOSITIONS

The present disclosure provides a method of delivering an exogenous antibody molecule that binds to HER2, to a subject. In some embodiments, the method comprising administering an effective amount of a pharmaceutical composition described herein or an AAV particle, e.g., a plurality of AAV particles, comprising an AAV vector, and/or a genetic element encoding an antibody molecule (e.g., an anti-HER2 antibody) described herein. In some embodiments, the subject has, has been diagnosed with having, or is at risk of having a disease or disorder, e.g., a disease associated with over-expression of HER2 (e.g., cancer and/or metastatic cancer).

The present disclosure also provides a method of treating a disease, disorder and/or condition in a subject, comprising administering to the subject any of the AAV particles described herein or administering to the subject an effective amount of a pharmaceutical composition described herein or an AAV particle, e.g., a plurality of AAV particles, comprising an AAV vector, and/or a genetic element encoding an antibody molecule (e.g., an anti-HER2 antibody) described herein.

In some embodiments, an AAV particle of the present disclosure is administered to a subject prophylactically. In some embodiments, an AAV particle of the present disclosure is administered to a subject having, diagnosed with having, or at risk of having a disease or disorder, e.g., a disease and/or a disorder associated with over-expression of HER2 (e.g., cancer and/or metastatic cancer).

In some embodiments, an AAV particle or a plurality of AAV particles of the present disclosure are part of an active immunization strategy to protect against a disease and disorder, e.g., a disease and/or a disorder associated with over-expression of HER2 (e.g., cancer and/or metastatic cancer). In some embodiments, in an active immunization strategy, a vaccine or an AAV particle is administered to a subject to prevent a disease by activating the subject's production of antibodies.

In some embodiments, an AAV particle or a plurality of AAV particles of the present disclosure are part of a passive immunization strategy. In some embodiments, in a passive immunization strategy, antibody molecules against a particular agent or antigen are given directly to the subject.

In some embodiments, an AAV particle or a plurality of AAV particles of the present disclosure may be used for passive immunotherapy of a disease and/or a disorder associated with over-expression of HER2 (e.g., cancer and/or metastatic cancer). As a non-limiting example, an AAV particles of the present disclosure may encode an anti-HER2 antibody molecule, e.g., as described in Tables 3A-3C. In some embodiments, an AAV particle may be administered by bilateral intraparenchymal delivery directly to the hippocampus.

Without wishing to be bound by theory, it is believed in some embodiments, that administration of an AAV particle or plurality of AAV particles of the present disclosure may result in substantially higher antibody levels in a cell (e.g., a neuronal cell or an endothelial cell) or tissue (e.g., a CNS tissue) of the subject, than if anti-HER2 antibody molecule were administered by passive immunization. While not wishing to be bound by theory, it is believed in some embodiments that passive immunization is thought to generate 20-40 ng of antibody per mg of protein in the brain of a subject. In some embodiments, AAV-mediated delivery of an antibody molecule results in antibody levels 2-5× above the levels observed, e.g., measured, with passive immunization. In some embodiments, AAV-mediated delivery of an antibody molecule results in antibody levels 1.5-3× above the levels observed, e.g., measured, with passive immunization. In some embodiments, AAV-mediated delivery of an antibody molecule results in antibody levels 5-10× above the levels observed, e.g., measured, with passive immunization. In some embodiments, AAV-mediated delivery of an antibody molecule results in antibody levels 8-16× above the levels observed, e.g., measured, with passive immunization.

### Diagnostic Applications

In some embodiments, an AAV particle of the present disclosure may be used for a diagnostic purpose or as diagnostic tools for any disease and/or disorder associated with HER2 over-expression (e.g., cancer and/or metastatic cancer). As non-limiting examples, an AAV particle of the present disclosure or the antibody molecule encoded by a genetic element therein may be used as a biomarker for disease diagnosis. As a second non-limiting example, an AAV particle of the present disclosure or the antibody molecule encoded by a genetic element therein may be used for diagnostic imaging purposes, e.g., MRI, PET, CT or ultrasound.

### Preventative Applications

In some embodiments, an AAV particle of the present disclosure or an antibody molecule encoded by a genetic element therein may be used to prevent a disease or stabilize progression of a disease. In some embodiments, an AAV particle of the present disclosure is used as a prophylactic to prevent a disease or disorder. In some embodiments, an AAV particle of the present disclosure is used to halt further progression of a disease or disorder. As a non-limiting example, an AAV particle may be used in a manner similar to that of a vaccine.

### Research Applications

In some embodiments, an AAV particle of the present disclosure or an antibody molecule encoded by a genetic element therein may be used as a research tool. In some embodiments, an AAV particle may be used as in any research experiment, e.g., in vivo or in vitro experiments. In a non-limiting example, an AAV particle may be used in a cultured cell, or plurality of cultured cells. The cultured cells may be produced from any origin known to one with skill in the art, and may be as non-limiting examples, produced from a stable cell line, an animal model or a human patient or control subject. In a non-limiting example, an AAV particle may be used in in vivo experiments in an animal model (e.g., a mouse, rat, rabbit, dog, cat, non-human primate, guinea pig, ferret, c-elegans, drosophila, zebrafish, or any other animal used for research purposes, known in the art). In another non-limiting example, an AAV particle may be used in a human research experiment or a human clinical trial.

### Therapeutic Applications

The present disclosure additionally provides a method of treating a disease associated with HER2 over-expression in a subject, e.g., a human subject, comprising administering to the subject any of the AAV particles described herein or administering to the subject an effective amount of a pharmaceutical composition described herein or an AAV particle, e.g., a plurality of AAV particles, comprising an AAV vector, and/or a genetic element encoding an antibody molecule (e.g., an anti-HER2 antibody) described herein. In some embodiments, a disease associated with HER2 over-expression comprises indications involving irregular cell proliferation. In some embodiments, treatment of a disease associated with HER2 over-expression comprises prevention of said disease associated with HER2 over-expression. In some embodiments, the subject has previously been treated for HER2+ cancer.

In some embodiments, the disease associated with HER2 over-expression in a subject is a HER2-positive cancer. Current methodologies for determining of HER2 status include immunohistochemistry (IHC) to detect HER2 protein overexpression, fluorescence in situ hybridization (FISH), or chromogenic in situ hybridization to detect amplification of the HER2 gene. A “HER2 positive” cancer, cancer cell, subject or patient, as used herein, refers to a cancer, cancer cell, subject, or patient exhibiting a score of at least 2 when using a HercepTest® (DakoCytomation California Inc., Carpenteria, Calif.) or a cancer, cancer cell, subject, or patient that has been identified as such by FISH, having a centromere 17 corrected HER2 gene copy number greater than 2 (HER2 FISH/CEP17>2; FISH positive). In certain embodiments, the HER2 positive cell exhibits a score of at least 2+ or 3+ using the HercepTest® Immunohistochemistry assay.

In some instances, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer.

In some embodiments, the HER2-positive cancer has metastasized to the central nervous system (CNS). In some instances, the metastasized HER2-cancer has formed CNS neoplasms. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, the HER2-positive cancer is breast cancer. Breast cancer is known to be a heterogeneous disease. There are different subtypes that can be defined based on (i) a molecular profile of a breast cancer tumor, (ii) gene array testing, or (iii) an immunohistochemical analysis approach. In some embodiments the breast cancer is Basal or Luminal subtype. In particular, mammary ducts are bilayered structures composed of a luminal layer and a myoepithelial layer that adhere to a basement membrane. The term basal refers to certain cancers that arise from the basal layer of the stratified epithelia. Breast carcinomas of the basal subtype reside in the basal layer of the ductal epithelium of the breast as opposed to the apical or luminal layers. Such cancers have distinct cytological features and gene expression profiles such as an intermediate filament profile (cytokeratins) first observed in the basal cells of the skin.

Most breast cancers are luminal tumors. Luminal tumor cells look like the cells of breast cancers that start in the inner (luminal) cells lining the mammary ducts. Of the four subtypes of luminal cancer, luminal A tumors tend to have the best prognosis, with fairly high survival rates and fairly low recurrence rates. Luminal B breast cancers are ER+ and/or PR+, HER2+ (or HER2− with high Ki67). About 6-17% of breast cancers are luminal B. Women with luminal B tumors are often diagnosed at a younger age than those with luminal A tumors. Compared to luminal A tumors, luminal B tumors also tend to have factors that lead to a poorer prognosis including: poorer tumor grade; larger tumor size; and p53 gene mutations.

In some embodiments, the breast cancer is ductal carcinoma in situ (intraductal carcinoma), lobular carcinoma in situ, invasive (or infiltrating) ductal carcinoma, invasive (or infiltrating) lobular carcinoma, inflammatory breast cancer, paget disease of the nipple, phyllodes tumor, angiosarcoma or invasive breast carcinoma. In some embodiments, the invasive breast carcinoma is further categorized into subtypes. In some embodiments, the subtypes include adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma or mixed carcinoma.

In some embodiments, the breast cancer is classified according to stages or how far the tumor cells have spread within the breast tissues and to other portions of the body. There are five stages of breast cancer, Stage 0-IV. The method of treating cancer described herein may be used to treat patients with breast cancer classified as Stage 0-Stage IV.

Stage 0 breast cancer refers to non-invasive breast cancers or that there are no evidence of cancer cells or abnormal non-cancerous cells breaking out of the origin site. Stage I breast cancer refers to invasive breast cancer in which the cancer cells have invaded into surrounding tissues. Stage I is subclassified into Stage IA and IB, in which Stage IA describes tumor measures up to 2 cm with no spread of cancer cells. Stage IB describes absence of tumor in breast but have small lumps of cancer cells between 0.2 mm to 2 mm within the lymph nodes. Stage II breast cancer is further subdivided into Stage IIA and IIB. Stage IIA describes tumor between 2 cm to 5 cm in breast only, or absence of tumor in breast but with cancer between 2 mm to 2 cm in axillary lymph nodes. Stage IIB describes tumor larger than 5 cm in breast only, or tumor between 2 cm to 5 cm in breast with presence of small tumors from 0.2 mm to 2 mm in axillary lymph nodes. Stage III breast cancer is further subdivided into Stage IIIA, IIIB, and IIIC. Stage IIIA describes absence of tumor or tumor greater than 5 cm in breast with small tumors in 4-9 axillary lymph nodes or small tumors 0.2 mm-2 mm in size in axillary lymph nodes. Stage IIIB describes tumor spreading into the chest wall or skin of the breast causing swelling or ulcer and with presence of tumor in up to 9 axillary lymph nodes. Inflammatory breast cancer is also considered as Stage IIIB. Stage IIIC describes absence of tumor or tumor spreading into the chest wall or to the skin of the breast, with tumor present in 10 or more axillary lymph nodes. Stage IV breast cancer refers to invasive breast cancer that has metastasized into the lymph nodes and other portions of the body.

In some embodiments, a method of treating a disease associated with HER2 over-expression in a subject in need thereof may comprise the steps of: (1) generating and/or selecting an anti-HER2 antibody molecule; (2) producing an AAV particle comprising a genetic element that comprises a nucleic acid comprising a transgene encoding an anti-HER2 antibody molecule of (1); and (3) administering the AAV particle (or pharmaceutical composition thereof) to the subject.

In some embodiments, the methods of treating a disease associated with HER2 over-expression herein may be used as adjuvant treatment. “Adjuvant treatment” is taken to mean a therapy of a cancer patient immediately following an initial non chemotherapeutical therapy, e.g. surgery, or radiation. In general, the purpose of an adjuvant therapy is to provide a significantly smaller risk of recurrences compared without the adjuvant therapy. For example, the subjects may have surgery or radiation therapy, after which they received treatment using the methods described herein. In some embodiments, the AAV particle herein may be administered to a subject concurrently with, or post a surgical resection of a HER2/neu-positive solid tumor. The administration may occur at the site of surgical resection (e.g., into a resection void) of a HER2/neu-positive solid tumor in the subject, alternatively, the administration may occur in the area around a site of surgical resection of a HER2/neu-positive solid tumor, e.g., the margins of the tumor, in the subject. Additionally, the AAV particle herein may be administered concurrently with, or post a radiation treatment (e.g., radiotherapy) of a HER2/neu-positive solid tumor.

The present disclosure provides a method for administering to a subject in need thereof, e.g., a human subject, a therapeutically effective amount of an AAV particle or a plurality of AAV particles described herein to prevent slow, stop, or reverse disease progression.

In certain embodiments, the progression of the disease (e.g., cancer) may be assessed by Response Evaluation Criteria in Solid Tumors (RECIST 1.1) (see Thereasse P., et al. New Guidelines to Evaluate the Response to Treatment in Solid Tumors. J. of the National Cancer Institute; 2000; (92) 205-216 and Eisenhauer E. A., Therasse P., Bogaerts J., et al. New response evaluation criteria in solid tumours: Revised RECIST guideline (version 1.1). European J. Cancer; 2009; (45) 228-247). Overall responses for all possible combinations of tumor responses in target and non-target lesions with or without the appearance of new lesions are as follows:

TABLE 29
Response Evaluation Criteria in Solid Tumors (RECIST 1.1)

| Target | Non-target | New | Overall |
|---|---|---|---|
| lesions | lesions | lesions | response |
| CR | CR | No | CR |
| CR | Incomplete response/SD | No | PR |
| PR | Non-PD | No | PR |
| SD | Non-PD | No | SD |
| PL | Any | Yes or no | PD |
| Any | PD | Yes or no | PD |
| Any | Any | Yes | PD |

CR = complete response; PR = partial response; SD = stable disease; and PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response (CR) is the disappearance of all non-target lesions and normalization of tumor marker level; incomplete response/stable disease (SD) is the persistence of one or more non-target lesion(s) and/or the maintenance of tumor marker level above the normal limits, and progressive disease (PD) is the appearance of one or more new lesions and/or unequivocal progression of existing non-target lesions.

In some embodiments, the subject may achieve CNS progression free survival as indicated in the RANO-BM criteria (Response Assessment in Neuro-Oncology Brain Metastases).

In some embodiments, the subject being treated with the AAV particles herein has experienced metastatic HER2+ breast cancer leptomeningeal spread to the central nervous system. In some embodiments, the AAV particles are administered IV to subjects experiencing metastatic HER2+ breast cancer leptomeningeal spread to the central nervous system.

In some embodiments, the progression of the disorder and/or disease associated with HER2 over-expression is delayed. The delaying of progression means to defer, hinder, slow, retard, stabilize, and/or postpone development of the disease or disorder (e.g., a HER2-positive cancer, e.g., a HER2-positive breast cancer or a HER2-positive metastatic cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

In some embodiments, the subject being treated for a disorder and/or disease associated with HER2 over-expression achieves a partial response. In some embodiments, the subject achieves a complete response. In some embodiments, the subject achieves progression free survival for a period of about 1 month, 2 months, 3 months. 4, months, 6 months, 7 months, 8 months, 9 months, 1 year, about 2 years, or about 3 years. In some embodiments, the subject achieves overall survival of about 1 year, about 2 years, about 3 years, about 4 years, about 5 years, 6 years, about 7 years, about 8 years, about 9 years, 10 years, about 15 years, or about 20 years.

In some embodiments, the disease associated with HER2 over-expression is a metastatic brain cancer. As another non-limiting example, disease progression may be measured by a change in the pathological features of the brain, CSF or other tissues of the subject, such as, but not limited to a decrease in the level of HER2 over-expressing cells or lesions. In some embodiments, the size of the lesion is decreased. In some embodiments, the lesions decrease in size, number, density, or a combination thereof.

In some embodiments, the method of treating a disease associated with HER2 over-expression in a subject, is used prophylactically. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells (e.g., HER2-positive cancer cells); reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder.

### Combination Applications

In some embodiments, an AAV particle may be used in combination therapy with an additional therapeutic agent and/or therapy, e.g., an additional therapeutic agent and/or therapy suitable for treatment or prevention of a disease associated with HER2 over-expression. In some embodiments, the therapeutic agent may utilize a therapeutic modality known in the art, e.g., gene silencing or interference (e.g., miRNA, siRNA, RNAi, shRNA), gene editing (e.g., TALEN, CRISPR/Cas9 systems, zinc finger nucleases), and gene, protein or enzyme replacement.

In some embodiments, the administration of an AAV particle described herein further comprises administration of chemotherapeutic agent, e.g., a cytotoxic chemotherapy pharmaceutical compound. Exemplary cytotoxic chemotherapy pharmaceutical compounds include, but are not limited to, a cyclophosphamide, doxorubicin, prednisolone, an ifosamide, a methotrexate, a substituted nucleotide, a substituted nucleoside, fluorouracil, a mitomycin, adriamycin, vincristine, vindesine, taxol, cisplatin, carboplatin, etoposide, or a combination thereof.

The AAV particle may be administered in addition to any of the standard treatments of metastatic breast cancer. In some instances, for subject experiencing HER2-positive metastatic breast cancer with brain lesions, the first line treatment can include trastuzumab, pertuzumab, and/or a chemotherapeutic agent. If the subject is unresponsive or unable to be treated with the first line treatment, they may be treated with the second line therapeutics, (e.g., trastuzumab emtansine), or third line therapeutics (e.g., trastuzumab, tucatinib, capecitabine, Fam-trastuzumab deruxtecan-nxki, Lapatanib/capecitabine, Lapatanib, Margetuxumab, a chemotherapeutic agent, Neratanib/capecitabine). The AAV particles of the present invention may be used with any of the first, second, or third line therapeutics. Additionally, the AAV particles of the present invention may be administered to a subject who has been unresponsive to the prior lines of treatment. In some embodiments, the subject has experienced refractory HER2-positive cancer.

In some embodiments, the administration of the AAV particle described herein further comprises administration of a non-vectorized therapeutic antibody and/or chemotherapeutic agent. For example, the AAV particles may be administered with trastuzumab, pertuzumab, a chemotherapeutic agent, or a combination thereof. Additionally, the administration may include trastuzumab emtansine; and/or trastuzumab, tucatinib, capecitabine, Fam-trastuzumab deruxtecan-nxki, Lapatanib/capecitabine, Lapatanib, Margetuximab, a chemotherapeutic agent, Neratanib/capecitabine, or a combination thereof.

In some embodiments, administration of an AAV particle described herein further comprises administration of an additional therapeutic agent and/or therapy suitable for treatment or prevention of a disease or disorder associated with HER2 over expression (e.g., cancer). In some embodiments, the additional therapeutic agent and/or therapy comprises a checkpoint inhibitor. In an embodiment, the checkpoint inhibitor is a biologic therapeutic or a small molecule. The checkpoint inhibitor can be a monoclonal antibody, a humanized antibody, a fully human antibody, a fusion protein or a combination thereof. The checkpoint inhibitor may inhibit a checkpoint protein selected from CTLA-4, PD-L1, PD-L2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands and combination thereof.

In some embodiments, any of the treatment regimens described herein include administration of a PD-1 axis binding antagonist (e.g., a PD-L1 binding antagonist, a PD-1 binding antagonist, or a PD-L2 binding antagonist). The PD-L1 binding antagonist may be an anti-PD-L1 antibody including, but not limited to MPDL3280A (atezolizumab), YW243.55.S70, MDX-1 105, and MEDI4736 (durvalumab), and MSB0010718C (avelumab). Antibody YW243.55. S70 is an anti-PD-L1 described in PCT Pub. No. WO 2010/077634, MDX-1 105, also known as BMS-936559, is an anti-PD-L1 antibody described in PCT Pub. No. WO 2007/005874, MEDI4736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference.

In some embodiments, the PD-1 binding antagonist is an anti-PD-1 antibody, such as an anti-PD-1 antibody selected from the group consisting of MDX-1 106 (nivolumab), MK-3475 (pembrolizumab), MEDI-0680 (AMP-514), PDR001, REGN2810, and BGB-108, MDX-1 1 06, also known as MDX-1 106-04, ONO-4538, BMS-936558, or nivolumab, is an anti-PD-1 antibody described in PCT Pub. No. WO 2006/121168, MK-3475, also known as pembrolizumab or lambrolizumab, is an anti-PD-1 antibody described in PCT Pub. No. WO 2009/114335. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In other instances, the PD-1 binding antagonist is AMP-224, AMP-224, also known as B7-DCIg, is a PD-L2-Fc fusion soluble receptor described in PCT Pub. Nos. WO 2010/027827 and WO 2011/066342.

In other instances, the PD-L2 binding antagonist is an anti-PD-L2 antibody (e.g., a human, a humanized, or a chimeric anti-PD-L2 antibody). In some instances, the PD-L2 binding antagonist is an immunoadhesin.

In a further embodiment, an additional therapeutic agent is an antibody-drug conjugate (ADC). In one embodiment, a AAV particle described herein is co-administered with an ADC selected from an anti-CD79b antibody drug conjugate (such as anti-CD79b-MC-vc-PAB-MMAE or the anti-CD79b antibody drug conjugate described in any one of U.S. Pat. No. 8,088,378 and/or US 2014/0030280, or polatuzumab vedotin).

In some instances, the additional therapy includes an alkylating agent. In one instance, the alkylating agent is 4-[5-[bis(2-chloroethyl)amino]-1-methylbenzimidazol-2-yl]butanoic acid and salts thereof. In one instance, the alkylating agent is bendamustine.

In some instances, the additional therapy comprises a BCL-2 inhibitor. In one embodiment, the BCL-2 inhibitor is 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide and salts thereof. In one instance, the BCL-2 inhibitor is venetoclax (CAS #: 1257044-40-8).

In some instances, the additional therapy comprises a phosphoinositide 3-kinase (PI3K) inhibitor. In one instance, the PI3K inhibitor inhibits delta isoform PI3K (i.e., P1 10d). In some instances, the PI3K inhibitor is 5-Fluoro-3-phenyl-2-[(1S)-1-(7H-purin-6-ylamino)propyl]-4(3H)-quinazolinone and salts thereof. In some instances, the PI3K inhibitor is idelalisib (CAS #: 870281-82-6). In one instance, the PI3K inhibitor inhibits alpha and delta isoforms of PI3K. In some instances, the PI3K inhibitor is 2-{3-[2-(1-isopropyl-3-methyl-1H-1,2-4-triazol-5-yl)-5,6-dihydrobenzo[f]imidazo[1,2-d][1,4]oxazepin-9-yl]-1H-pyrazol-1-yl}-2-methylpropanamide and salts thereof.

In a further aspect of the invention, the additional therapy comprises a Bruton's tyrosine kinase (BTK) inhibitor. In one instance, the BTK inhibitor is 1-[(3R)-3-[4-Amino-3-(4-phenoxyphenyl)-1H-pyrazolo[3,4-d]pyrimidin-1-yl]piperidin-1-yl]prop-2-en-1-one and salts thereof. In one instance, the BTK inhibitor is ibrutinib (CAS #: 936563-96-1).

In some instances, the additional therapy comprises thalidomide or a derivative thereof. In one instance, the thalidomide or a derivative thereof is (RS)-3-(4-amino-1-oxo-1,3-dihydro-2H-isoindol-2-yl)piperidine-2,6-dione and salts thereof. In one instance, the thalidomide or a derivative thereof is lendalidomide (CAS #: 191 732-72-6).

In one instance, the additional therapeutic agent is a corticosteroid, which can be administered as a pre-treatment prior to (e.g., about 1 hour prior to) administration of the AAV particle described herein. Corticosteroid premedication can include administration of dexamethasone or methylprednisolone. Additionally or alternatively, the treatment regimens described herein may include administration of (e.g., pre-treatment with) acetaminophen, paracetamol, or diphenhydramine.

In some embodiments, an IL-6R antagonist, such as tocilizumab (ACTEMRA®/RoACTEMRA®) may be administered, e.g., if necessary to manage a cytokine release syndrome (CRS) event. In particular embodiments, an IL-6R antagonist (e.g., tocilizumab) may be administered intravenously, e.g., at a dose of 1 mg/kg to 25 mg/kg (e.g., from 5 mg/kg to 10 mg/kg, e.g., about 8 mg/kg), as necessary.

In some embodiments, in addition to the AAV particle described herein, the subject is administered supportive care, for example, pain medication for headaches, treatment for infusion-related reactions (IRRs), and prophylaxis for infusion-related reactions. Symptoms for IRRs include, for example, flushing, alterations in heart rate and blood pressure, dyspnea, bronchospasm, back pain, fever, urticaria, edema, nausea, and rashes. Exemplary treatments for an IRR include, but are not limited to acetaminophen, IV hydration, diphenhydramine, histamine₂ blockers (e.g., famotidine), and corticosteroids.

### VI. KITS AND DEVICES

### Kits

In some embodiments, the disclosure provides a kit for conveniently and/or effectively carrying out methods of the present disclosure. In some embodiments, a kit comprises a sufficient amount and/or number of components to perform multiple treatments of a subject(s) and/or to perform multiple experiments.

In some embodiments, an AAV particle described herein may be comprised in a kit. In some embodiments, a kit may further include reagents and/or instructions for creating and/or synthesizing compounds and/or compositions of the present disclosure. In some embodiments, a kit may also comprise one or more buffers. In some embodiments, a kit may comprise a component for making a protein or nucleic acid array or library and thus, may include, for example, a solid support.

In some embodiments, a kit component may be packaged either in aqueous media or in lyophilized form. In some embodiments, a container used in a kit described herein comprises a vial, test tube, flask, bottle, syringe, wherein a component may be placed, and/or suitably aliquoted. In some embodiments, a kit may comprise one or more components, e.g., labeling reagent and label may be packaged together. In some embodiments, a kit may also contain a second, a third or an additional container into which additional components may be separately placed. In some embodiments, a kit may also comprise second container for containing sterile, pharmaceutically acceptable buffers and/or other diluents. In some embodiments, various combinations of components may be comprised in one or more vials. In some embodiments, a kit of the present disclosure may also include means for containing a compound and/or composition of the present disclosure, e.g., a protein, nucleic acid, and any other reagent container in close confinement for commercial sale. Such a container may include injection or blow-molded plastic containers into which desired vials are retained.

In some embodiments, kit components are provided in one and/or more liquid solutions. In some embodiments, a liquid solution is an aqueous solutions. In some embodiments, the aqueous solution comprises a sterile aqueous solution. In some embodiments, a kit components may be provided as a dried powder. In some embodiments, when a reagent and/or a component are provided as a dry powder, such a powder may be reconstituted by the addition of suitable volumes of solvent. In some embodiments, a solvent may also be provided in another container. In some embodiments, a labeling dye is provided as a dried powder. In some embodiments, at least 10, 20, 30, 40, 50, 60, 70, 80, 90, 100, 120, 120, 130, 140, 150, 160, 170, 180, 190, 200, 300, 400, 500, 600, 700, 800, 900, 1000 micrograms of a dried dye are provided in a kit described herein. In such embodiments, a dye may then be resuspended in any suitable solvent, such as DMSO.

In some embodiments, a kits may include instructions for use the kit components as well the use of any other reagent not included in the kit. In some embodiments, the instructions may include variations that may be implemented.

### Devices

In some embodiments, an AAV particle may delivered to a subject using a device to deliver the AAV particle and a head fixation assembly. In some embodiments, the head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569, and 9,031,636 and International Patent Publication Nos. WO201108495 and WO2014014585, the contents of each of which are incorporated by reference in their entireties. In some embodiments, A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, an AAV particle may be delivered using a method, system and/or computer program for positioning apparatus to a target point on a subject to deliver the AAV particle. As a non-limiting example, the method, system and/or computer program may be the methods, systems and/or computer programs described in U.S. Pat. No. 8,340,743, the contents of which are herein incorporated by reference in its entirety. In some embodiments, the method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, an AAV particle may be delivered to a subject using a convention-enhanced delivery device. Non-limiting examples of targeted delivery of drugs using convection are described in US Patent Publication Nos. US20100217228, US20130035574, and US 20130035660 and International Patent Publication No. WO2013019830 and WO2008144585, the contents of each of which are herein incorporated by reference in their entireties.

In some embodiments, a subject may be imaged prior to, during and/or after delivery of an AAV particle. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles. In some embodiments, an AAV particle may be delivered intraparenchymal to a subject and used for intra-operative MRI during surgery to visualize delivery.

In some embodiments, an AAV particle may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677, and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, an AAV particle may be delivered using an MRI-compatible tip assembly. Non-limiting examples of MRI-compatible tip assemblies are described in US Patent Publication No. US20140275980, the contents of which is herein incorporated by reference in its entirety.

In some embodiments, an AAV particle may be delivered using a cannula which is MRI-compatible. Non-limiting examples of MRI-compatible cannulas include those taught in International Patent Publication No. WO2011130107, the contents of which are herein incorporated by reference in its entirety.

In some embodiments, an AAV particle may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In some embodiments, an AAV particle may be delivered using a device with an elongated tubular body and a diaphragm as described in US Patent Publication Nos. US20140276582 and US20140276614, the contents of each of which are herein incorporated by reference in their entireties.

In some embodiments, an AAV particle may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, an AAV particle may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, an AAV particle may be delivered to a subject using a gene gun.

### VII. EXAMPLES

### Example 1. Generation of Anti-HER2 Monospecific Antibody Molecules

Genetic element constructs were designed for AAV delivery of anti-HER2 antibodies. The nucleotide sequences from 5′ ITR to 3′ ITR of the genetic element constructs that comprise a transgene encoding a monospecific HER2 antibody molecule are provided as HER-04, HER-05, HER-10, HER-42, HER-43, HER-46, HER-47, HER-53, HER-57, HER-62, HER-75, HER-88, HER-89, HER-90, HER-91, and HER-92 herein, which are SEQ ID NOs: 5163, 5170, 5164, 5166, 5185, 5186, 5167, 5168, 5187, 5188, 5190, 6500, 6501, 6502, 6503, and 6504, respectively. These constructs are also summarized in Table 6, as well as Tables 68, 17, 18, 19, 24 and 26.

Each of these genetic element constructs comprise a nucleic acid comprising a transgene encoding an antibody molecule that binds to HER2. The transgene was designed to comprise a nucleotide sequence encoding an antibody heavy chain signal sequence (SEQ ID NO:5157), a nucleotide sequence encoding an antibody heavy chain variable region (VH) (SEQ ID NO: 5002, 5171, 5015, 5070, 5191, 5223, 5089, 5109, 5253, 5257, 5261, 6512, 6517, 6522, 6527, or 6532), a nucleotide sequence encoding antibody heavy chain constant region (SEQ ID NO: 5003, 5011, 5017, 5025, 5211, 5213, 5029, or 5219), a nucleotide sequence encoding a linker region, a nucleotide sequence encoding an antibody light chain signal sequence (SEQ ID NO: 5159), a nucleotide sequence encoding an antibody light chain variable region (VL) (SEQ ID NO: 5005, 5175, 5019, 5085, 5195, 5227, 5093, 5113, 5255, 5259, or 5265), and a nucleotide sequence encoding an antibody light chain constant region (SEQ ID NO: 5007, 5021, 5277, 5279, or 5221). The linker region was a T2A linker (SEQ ID NO: 1726), which was flanked by a furin cleavage site (SEQ ID NO: 1724) at the 5′ end of the T2A linker. Alternatively, the linker region for HER-57 and HER-62 was, from 5′ to 3′, a series of G4S (SEQ ID NO: 4535) linkers (SEQ ID NOS: 6597, 5161, and 5162).

The encoded antibodies of HER-04, HER-05, HER-10, HER-42, HER-43, HER-46, HER-47, HER-53, HER-57, HER-62, HER-75, HER-88, HER-89, HER-90, HER-91, and HER-92 comprise a heavy chain signal sequence (SEQ ID NO:5158), an antibody heavy chain variable region (VH) (SEQ ID NO: 5002, 5172, 5016, 5071, 5192, 5224, 5090, 5110, 5254, 5258, 5262, 6511, 6516, 6521, 6526, or 6531), an antibody heavy chain constant region (SEQ ID NO: 5004, 5012, 5018, 5026, 5212, 5214, 5030, or 5220), an antibody light chain signal sequence (SEQ ID NO: 5160), an antibody light chain variable region (VL) (SEQ ID NO: 5006, 5176, 5020, 5086, 5196, 5228, 5094, 5114, 5256, 5260, or 5266), and an antibody light chain constant region (SEQ ID NO: 5008, 5022, 5278, 5280, or 5222).

### Example 2. Codon Optimization of Sequences Encoding an Anti-HER2 Monospecific Antibody Molecule

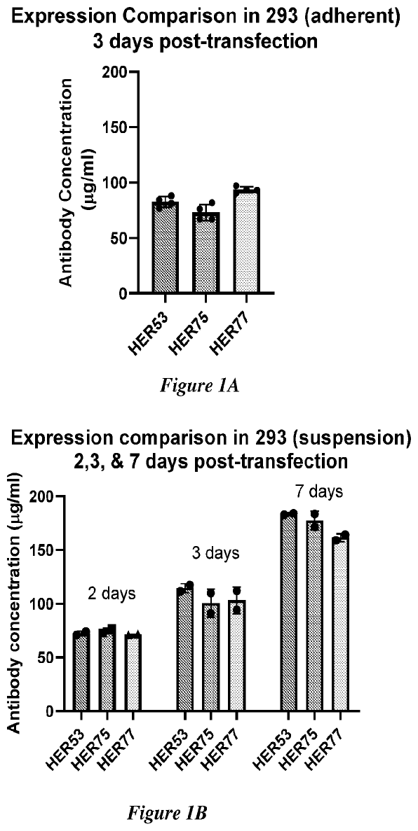

Unmethylated CpG dinucleotide-based motifs in AAV transgenes are known to stimulate innate immune responses by binding and activating Toll-Like receptor (TLR9) signaling pathway. Anti-HER2 monospecific antibody molecule HER-53 was further codon optimized to eliminate a total of 87 dinucleotides in the transgene. The expression of the resulting codon optimized HER-75 was compared to HER-53 and HER-77 in HEK expi293 cells. Conditioned media were taken 2, 3, and 7 days post-transfection and subject to IgG quantification with a Biacore 8K system. FIGS. 1A and 1B show the comparison of the expression of the three monospecific antibody molecules in HEK cells.

HEK-Blue human TLR9 (hTLR9) cells (invivogen hkb-htlr9) overexpress hTLR9 gene. They also express a secreted embryonic alkaline phosphatase (SEAP) reporter gene under the control of an NF-kB inducible promoter. Levels of SEAP in the culture supernatant reflect the level of TLR9 induced NF-kB signaling. HEK-Blue hTLR9 cells were used to study potential immunogenicity of HER-53 and HER-75 transgenes. Briefly, HER-53 and HER-75 transgene fragments were cut and separated from their vector parts with restriction enzymes AgeI/XhoI and 1.2% agarose gel. Transgene DNA fragments were recovered from agarose gel and transfected to HEK-Blue hTLR9 cells with FuGENE HD (Promega E2311). Levels of SEAP in the conditioned media 5 days post-transfection were measured with QUANTI-Blue solution (Invivogen: rep-qbs) (FIG. 1C).

### Example 3. Generation of ITR-ITR Constructs for the Expression of Anti-HER2 Monospecific Antibody Molecules

In designing the genetic element constructs for expression of monospecific HER2 antibody molecules (HER-04, HER-05, HER-10, HER-42, HER-43, HER-46, HER-47, HER-53, HER-57, HER-62, HER-75, HER-69, HER-88, HER-89, HER-90, HER-91, and HER-92), several promoters were selected. The ubiquitous CB promoter (SEQ ID NO: 6566) was used for HER-04 (SEQ ID NO: 5163), HER-05 (SEQ ID NO: 5170), HER-43 (SEQ ID NO: 5185), HER-46 (SEQ ID NO: 5186), HER-62 (SEQ ID NO: 5188), HER-75 (SEQ ID NO: 5190), HER-88 (SEQ ID NO: 6500), HER-89 (SEQ ID NO: 6501), HER-90 (SEQ ID NO: 6502), HER-91 (SEQ ID NO: 6503), and HER-92 (SEQ ID NO: 6504). The ubiquitous CB promoter (SEQ ID NO: 6566) further comprising a CMV(IE) promoter (SEQ ID NO: 6594), was used for HER-10 (SEQ ID NO: 5164), HER-42 (SEQ ID NO:5166), HER-47 (SEQ ID NO:5167), HER-53 (SEQ ID NO: 5168), and HER-57 (SEQ ID NO: 5187). The astrocyte targeting GFAP promoter (SEQ ID NO: 6568) was used for HER-69 (SEQ ID NO: 5169).

The HER-04, HER-05, HER-43, HER-46, HER-57, HER-62, HER-75, HER-88, HER-89, HER-90, HER-91, and HER-92 genetic elements further comprised an ie exon 1 region (SEQ ID NO: 6573), an ie intron 1 region (SEQ ID NO: 6578), a second human beta-globin intron region (SEQ ID NO: 6580), and a human beta-globin exon region (SEQ ID NO: 6576). The HER-10, HER-42, HER-47, and HER-53 genetic elements comprising a CB promoter, as well as the HER-69 genetic elements comprising a GFAP promoter, further comprised human beta-globin intron region (SEQ ID NO: 6595).

The 5′ ITR (SEQ ID NO: 6559), the polyadenylation sequence (SEQ ID NO: 6590), and the 3′ ITR (SEQ ID NO: 6561) were the same across all HER-04, HER-05, HER-10, HER-42, HER-43, HER-46, HER-47, HER-53, HER-57, HER-62, HER-75, HER-69, HER-88, HER-89, HER-90, HER-91, and HER-92 genetic element constructs designed for the expression of an anti-HER2 antibody molecule.

All sixteen genetic element constructs and their component parts are summarized in Tables 6, 68, 17, 18, 19, 23, 24, and 26 above.

### Expression of Anti-HER2 Antibodies—Validation of Cell Lines

HER2 expression in a panel of cancer cell lines was analyzed by flow cytometry. Briefly, the cells were dissociated using StemPro Accutase (Gibco A1110501), and the pelleted cells were washed with FACS Buffer (1-2% BSA in PBS). For cell surface HER2 expression, the cells were incubated with a PE-conjugated mouse anti-HER2 antibody (BD 340879) or the appropriate isotype control antibody (BD 559320) for 30 minutes on ice in the dark. Following antibody incubation, the cells were washed twice with FACS Buffer and resuspended in cold FACS Buffer. The cells were passed through 35 μm cell strainers prior to analysis on a BD Accuri C6 flow cytometer.

HER2 expression was found to be elevated in the following cell lines compared to the negative control U87-MG (glioblastoma) cell line: BT-474 (breast), SK-BR-3 (breast), MDA-MB-361-luc (breast, brain metastasis), and SK-OV-3 (ovarian).

### Expression of Anti-HER2 Antibodies in Cancer Cells

HER2 antibody secretion by the SK-BR-3 breast cancer cell line three days post-transfection with the HER-04, HER-05, HER-10, and HER-15 genetic element constructs was quantified via an AlphaLISA assay for human IgG1. Prior to reverse transfection with Lipofectamine LTX, a 24-well plate was coated with 10 μg/mL of fibronectin (Sigma-Aldrich F1141). SK-BR-3 cells were plated at 200,000 cells/well along with 500 ng of plasmid DNA complexed with Lipofectamine LTX and PLUS Reagent. The culture medium was replaced with serum-free Opti-MEM one day post-transfection. Three days post-transfection with the HER-04, HER-05, HER-10, and HER-15 genetic element constructs, the media was collected from the cells and analyzed using the human IgG1 AlphaLISA Detection Kit (Perkin Elmer AL307) following manufacturer protocols. Briefly, 5 μL of culture supernatant was added to a white 384-well plate and incubated with AlphaLISA beads in a 50 uL final assay volume. AlphaLISA readings were taken on a PerkinElmer EnVision 2105 Multimode Plate Reader. The standard curve and data interpolation were analyzed in GraphPad Prism using a sigmoidal 4PL fit with 1/Y² data weighting. The concentration of the antibodies expressed by the SK-BR-3 breast cancer cells three days post-transfection in shown in Table 30 below.

TABLE 30
Concentration of human IgG1 by transfected
SK-BR-3 breast cancer cells

| SK-BR-3 Transfection | Human IgG1 (ng/mL) |
|---|---|
| Human IgG1 isotype control antibody | 319.5 |
| HER-04 | 537.0 |
| HER-05 | 428.0 |
| HER-10 | 377.0 |
| HER-15 | 11.0 |

### Expression of Anti-HER2 Antibodies—Western Blot

Expression of the heavy chains and light chains of HER2 antibody clones produced by the HER-04, HER-05, HER-10, and HER-15 genetic element constructs was evaluated by Western blot. SK-BR-3 cells were reverse transfected with plasmid DNA using Lipofectamine LTX and PLUS Reagent in fibronectin-coated plates. Culture supernatant was replaced with serum-free medium (Opti-MEM or Cancer Cell Line Medium XF, PromoCell C-28077) one day post-transfection. Three days post-transfection, the culture medium was concentrated using centrifugal concentrators (MilliporeSigma) or purified using Protein A agarose (Thermo Scientific 22811) prior to SDS-PAGE using a MES SDS Running Buffer (Invitrogen). Proteins were separated through 4-12% Bis-Tris gels and processed through silver staining (Thermo Scientific 24612), or transferred to nitrocellulose membranes for Western blotting with a fluorophore-conjugated goat anti-human antibody (LI-COR 925-32232) at 1:10,000 dilution. Silver stains were imaged on a Bio-Rad ChemiDoc MP Imaging System, and Western blots were imaged on a LI-COR Odyssey CLx. Secreted HER-04, HER-05, human IgG1 isotype control antibody, HER-10, and HER-15 antibodies from transfected SK-BR-3 breast cancer cells were analyzed by Western blot and silver stain to confirm that the antibodies can fully assemble, and subsequently be reduced to monomeric heavy and light chains. Cells expressing the HER-04, HER-05, and HER-10 antibodies were able to successfully produce the heavy and light chains of the encoded anti-HER2 antibody, as evidenced by bands at 50 kDA and 25 kDa, respectively. HER-15 was poorly expressed/secreted by SK-BR-3 cells.

Expression of the heavy chains and light chains of HER-10 (control), HER-53, HER-75, HER-88, HER-89, HER-90, HER-91, and HER92 genetic element constructs was evaluated by Western blot as described above. Secreted human IgG1 isotype control antibody, HER-53, HER-75, and heavy chain mutants HER-88, HER-89, HER-90, HER-91, and HER-92 from transfected SK-BR-3 breast cancer cells were analyzed by Western blot and silver stain to confirm that the antibodies can fully assemble, and subsequently be reduced to monomeric heavy and light chains. Cells expressing the antibodies were able to successfully produce the heavy and light chains of the encoded anti-HER2 antibody.

### Example 4. In Vitro Characterization of Anti-HER2 Monospecific Antibody Molecules

### HER2 Binding Affinity Measurement

Antibody binding to HER2 was analyzed by Biacore 8K (GE Healthcare) surface plasmon resonance instrument (SPR). All reagents used were from GE Healthcare. HBS-EP+ was used as the sample and running buffer and the interaction was measured at 25° C. A CM5 sensor chip was immobilized with anti-human IgG Fc antibody, as per protocol suggested by Human Antibody Capture kit (GE Healthcare). The Anti-HER2 antibodies were diluted to 0.5 μg/ml in HBS-EP+ and were captured by flowing for 120 sec at 10 μl/min to give a final HER2 binding response of less than 200 RU. HER2 ECD (0.625-80 nM, 2-fold dilution) was passed over the captured anti-HER2 antibody at 30 μl/min flow rate, and association and dissociation phases were recorded for 300 and 600 sec, respectively. The sensor chip was regenerated by a 30 sec injection of 3M MgCl₂ at 30 μl/min at the end of each injection cycle. The sensorgrams were fitted to 1:1 binding model (global fit) in the Biacore Insight Evaluation Software to calculate kinetic rate constants and binding affinity value. For anti-HER2 antibodies with FcRn mutations (HER-43, HER-46, and HER-47), antibodies in conditioned media were used. The conditioned media was diluted in HBS-EP+ to achieve a capture level that produced a final HER2 binding response of less than 200 RU.

The Bonding affinity data is shown in Table 31 below. Binding affinity of the vectorized antibody was found to be comparable to the recombinant antibody, suggesting that vectorization process does not affect the ability of antibody to bind to HER2. The FcRn-binding mutants (HER-43, HER-46, and HER-47) showed a slightly weaker affinity for HER2, and the bispecific antibody, Her-73 showed a stronger HER2 binding affinity.

TABLE 31
Binding affinity to HER2

| | | | |
|---|---|---|---|
| Antibody | K_{D} (nM) | kon (1/Ms) | koff (1/s) |

Recombinant (Naked) Antibody

| | | | |
|---|---|---|---|
| HER-04 | 1.5 | 1.3E+05 | 1.9E−04 |
| HER-05 | 2.7 | 8.7E+04 | 2.3E−04 |
| HER-10 | 1.2 | 1.9E+05 | 2.1E−04 |

Vectorized Antibody

| | | | |
|---|---|---|---|
| HER-04 | 1.7 | 1.3E+05 | 2.2E−04 |
| HER-05 | 2.7 | 8.1E+04 | 2.2E−04 |
| HER-10 | 1.5 | 1.7E+05 | 2.5E−04 |
| HER-15 | 0.3 | 4.3E+05 | 1.2E−04 |
| HER-53 | 1.6 | 1.1E+05 | 1.7E−04 |

FcRn binding mutants

| | | | |
|---|---|---|---|
| HER-43 | 6.2 | 1.1E+05 | 6.8E−04 |
| HER-46 | 3.5 | 1.0E+05 | 3.6E−04 |
| HER-47 | 7.8 | 1.2E+05 | 9.1E−04 |

Bispecific antibody molecule

| | | | |
|---|---|---|---|
| HER-73 | 0.2 | 1.3E+05 | 2.4E−05 |

### Heavy Chain CDR3 Mutant Binding Affinity Measurements

The antibody binding of the Heavy chain CDR3 mutants (HER-88, HER-89, HER-90, HER-91, HER-92) to HER2 was analyzed by Biacore 8K (GE Healthcare) surface plasmon resonance instrument (SPR) as described above.

TABLE 47
Binding affinity of Heavy CDR3 mutants to HER2

| | HCDR3 | | | | | | | Fit quality parameters | |
|---|---|---|---|---|---|---|---|---|---|
| | Modifications | | | | | | | | chi2/Rmax |
| | (according to | | Kinetic parameters | | | | | Kinetics | (%) (Calc. |
| Construct | Kabat) | Replicate | KD (nM) | ka (1/Ms) | kd (1/s) | KD (M) | Rmax (RU) | Chi² (RU²) | unit) |
| Her53 | D98 M100, | 1 | 1.81 | 1.03E+05 | 1.86E−04 | 1.81E−09 | 88.8 | 0.67 | 0.8% |
| | D101, Y102 | 2 | 1.96 | 1.01E+05 | 1.97E−04 | 1.96E−09 | 89.1 | 0.81 | 0.9% |
| | | Mean | 1.89 | 1.02E+05 | 1.92E−04 | 1.89E−09 | 88.95 | | |
| | | % CV | 5.6 | 1.4 | 4.1 | 5.6 | 0.2 | | |
| Her75 | D98 M100, | 1 | 1.25 | 1.23E+05 | 1.54E−04 | 1.25E−09 | 83.9 | 1.15 | 1.4% |
| | D101, Y102 | 2 | 1.43 | 1.18E+05 | 1.69E−04 | 1.43E−09 | 85.6 | 1.59 | 1.9% |
| | | Mean | 1.34 | 1.21E+05 | 1.62E−04 | 1.34E−09 | 84.75 | | |
| | | % CV | 9.5 | 2.9 | 6.6 | 9.5 | 1.4 | | |
| Her88 | D98W, | 1 | 0.87 | 1.10E+05 | 9.54E−05 | 8.69E−10 | 59.9 | 0.65 | 1.1% |
| | M100F, | 2 | 1.01 | 1.01E+05 | 1.03E−04 | 1.01E−09 | 62.9 | 0.72 | 1.1% |
| | D101A, | Mean | 0.94 | 1.06E+05 | 9.92E−05 | 9.40E−10 | 61.40 | | |
| | Y102L | % CV | 10.6 | 6.0 | 5.4 | 10.6 | 3.5 | | |
| Her89 | D98W, | 1 | 1.35 | 9.90E+04 | 1.34E−04 | 1.35E−09 | 53.7 | 0.84 | 1.6% |
| | M100F, | 2 | 1.49 | 9.70E+04 | 1.45E−04 | 1.49E−09 | 53.4 | 0.73 | 1.4% |
| | D101A | Mean | 1.42 | 9.80E+04 | 1.40E−04 | 1.42E−09 | 53.55 | | |
| | | % CV | 7.0 | 1.4 | 5.6 | 7.0 | 0.4 | | |
| Her90 | D98W, | 1 | 1.46 | 1.15E+05 | 1.68E−04 | 1.46E−09 | 57.6 | 1.00 | 1.7% |
| | M100F, | 2 | 1.58 | 1.08E+05 | 1.71E−04 | 1.58E−09 | 59.5 | 0.83 | 1.4% |
| | Y102L | Mean | 1.52 | 1.12E+05 | 1.70E−04 | 1.52E−09 | 58.55 | | |
| | | % CV | 5.6 | 4.4 | 1.3 | 5.6 | 2.3 | | |
| Her91 | D98W, | 1 | 2.18 | 1.32E+05 | 2.88E−04 | 2.18E−09 | 60.7 | 0.90 | 1.5% |
| | D101A, | 2 | 1.82 | 1.41E+05 | 2.57E−04 | 1.82E−09 | 59.9 | 0.97 | 1.6% |
| | Y102L | Mean | 2.00 | 1.37E+05 | 2.73E−04 | 2.00E−09 | 60.30 | | |
| | | % CV | 12.7 | 4.7 | 8.0 | 12.7 | 0.9 | | |
| Her92 | M100F, | 1 | 1.16 | 1.19E+05 | 1.38E−04 | 1.16E−09 | 73.2 | 1.45 | 2.0% |
| | D101A, | 2 | 1.24 | 1.11E+05 | 1.38E−04 | 1.24E−09 | 75.9 | 0.91 | 1.2% |
| | Y102L | Mean | 1.20 | 1.15E+05 | 1.38E−04 | 1.20E−09 | 74.55 | | |
| | | % CV | 4.7 | 4.9 | 0.0 | 4.7 | 2.6 | | |

### FcRn Binding Affinity Measurement

Antibody binding to the FcRn receptor at pH 6.0 and 7.4 was analyzed by Biacore 8K (GE Healthcare) surface plasmon resonance instrument (SPR). All reagents used were from GE Healthcare. For the pH 6.0 assay, PBS-P+ buffer was adjusted to pH 6.0 and supplemented with NaCl to a final concentration of 500 mM. For the pH 7.4 assay, PBS-P+ was used. Biotinylated human and mouse FcRn receptor (Acro Biosystems) were captured on a Biotin CAPture chip by injecting 1 μg/ml of FcRn for 30 sec at 10 μl/min. The Anti-HER2 antibody was injected (12-3000 nM, 3-fold dilution) over the captured FcRn in a single cycle kinetics (SCK) mode with an association and dissociation time of 60 and 120 sec, respectively. The sensor chip was regenerated according to manufacturer' instructions. Sensorgrams were fitted to steady-state affinity model for human FcRn and 1:1 model for mouse FcRn to calculate the affinity values.

At pH 6.0, HER-04 showed an affinity value of K_{D}=299 nM and 49 nM for human and mouse FcRn, respectively, while FcRn binding mutants, HER-43, HER-46, and HER-47 showed no binding to FcRn. At pH 7.4, no binding was observed for all antibodies.

### Ability of Anti-HER2 Antibodies Expressed by SK-BR-3 and Expi293 Cells to Bind HER2

The ability of the anti-HER2 antibodies expressed by SK-BR-3 and Expi293 cells transfected with the HER-10 genetic element construct to bind HER2 was assessed. SK-BR-3 cells were reverse transfected with plasmid DNA using Lipofectamine LTX and PLUS Reagent in fibronectin-coated flasks. The culture supernatant was replaced with serum-free medium (Opti-MEM) one day post-transfection. Three days post-transfection, the culture medium was concentrated using centrifugal concentrators (MilliporeSigma) and analyzed by Biacore SPR binding to Her2 ECD with data fitted to a 1:1 binding model (global fit). The binding affinity of the HER2 antibody expressed from SK-BR-3 breast cancer cells was comparable to the anti-HER2 antibody expressed from Expi293 cells, with KD values of 0.96 nM and 1.58 nM, respectively, as assessed by Biacore SPR.

### Efficacy Measurement of Constructs—Cell Proliferation Assays

### BT-474 Cell Proliferation Assays

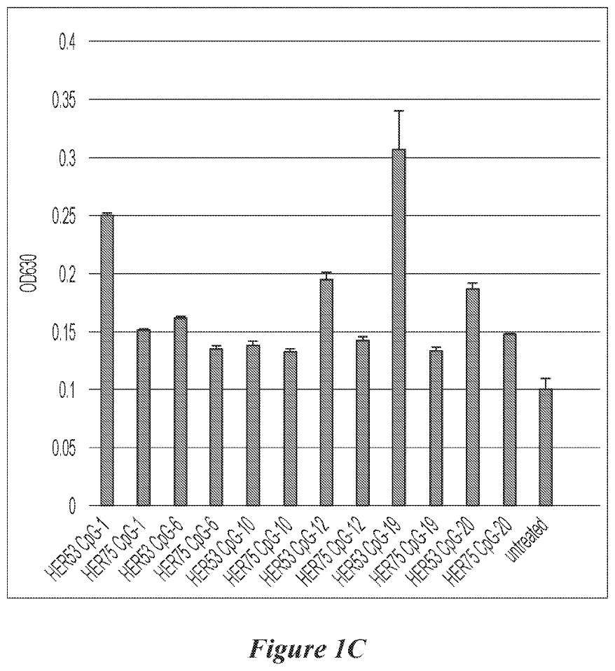

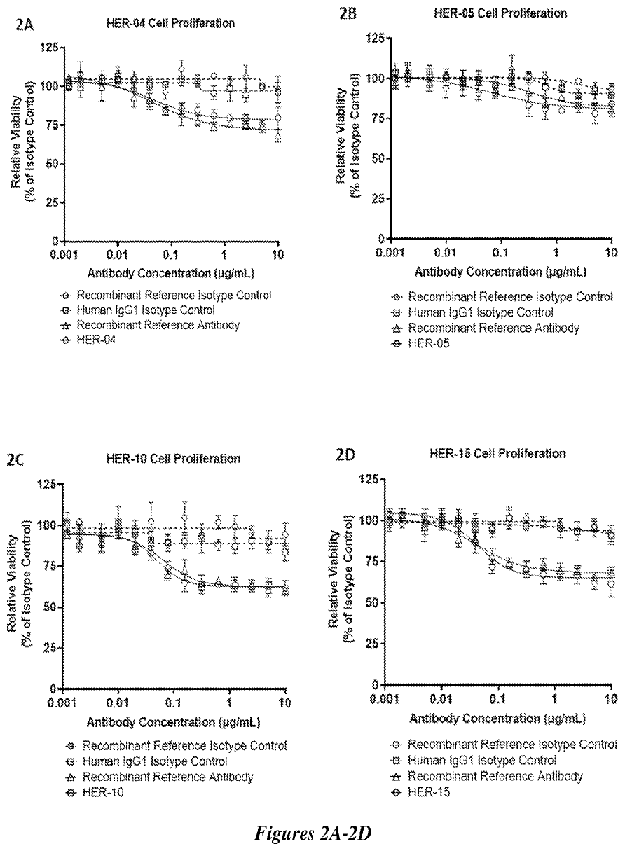

BT-474 cells were dispensed at 625 cells/well using a Multidrop Combi Reagent Dispenser (Thermo Scientific) into white 384-well plates pre-dispensed with antibody dilutions. A two-fold serial dilution series was prepared for each antibody in sterile polypropylene 96-well plates. Assay plates were covered with Microclime Environmental Lids (Beckman Coulter 0015717) filled with 8 mL of UltraPure Distilled water (Invitrogen 10977-015) to reduce well evaporation. Cell proliferation was measured using the CellTiter-Glo 2.0 assay (Promega G9242) after four days of antibody treatment. Luminescence measurements were taken on an EnVision 2105 Multimode Plate Reader, and data were analyzed in GraphPad Prism using a variable slope (four parameters) fit with 1/Y² data weighting. Edge wells were excluded from analysis. The plots of the cell proliferation assay for the vectorized anti-HER2 antibodies HER-04, HER-05, HER-10, and HER-15 are shown in FIGS. 2A-2D, respectively. The vectorized anti-HER2 antibodies expressed by Expi293 cells performed similar to reference antibodies (Creative Biolabs) in cell growth inhibition assays with BT-474 cells. Additionally, similar cell growth inhibition was observed between the HER-53 antibody with an enhanced Fc region and HER-04 (FIG. 3, Table 49). HER-10 performed better than HER-53 or HER-04 (FIG. 3).

TABLE 49
IC₅₀ and Eₘₐₓ HER-53, HER-04, and HER-10 in BT-474 cells

| Antibody | IC₅₀ (μg/mL) | Eₘₐₓ (%) |
|---|---|---|
| HER-53 | 0.083 | 28.7 |
| HER-04 | 0.058 | 35.1 |
| HER-10 | 0.041 | 38.0 |

### MDA-MB-361-Luc Cell Proliferation Assays

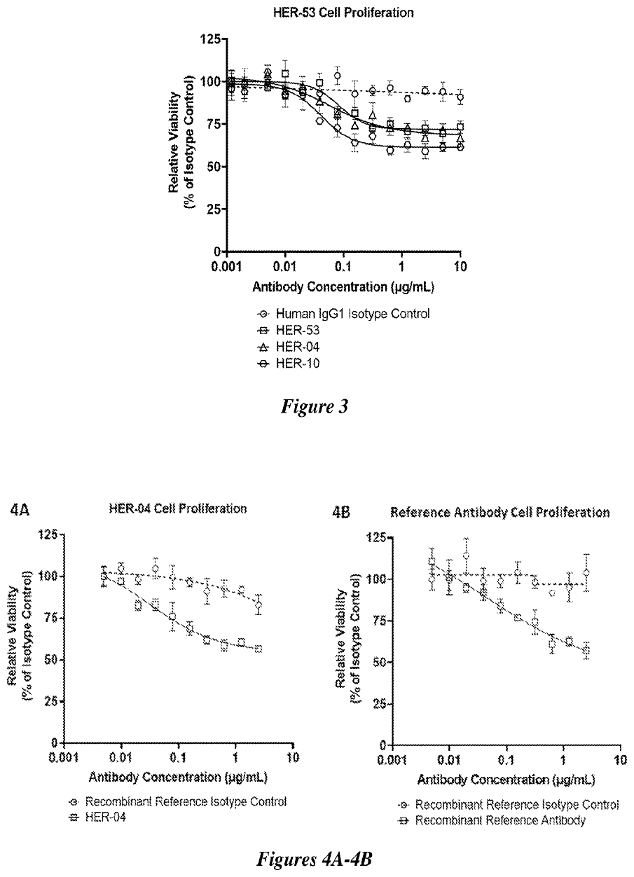

MDA-MB-361-luc cells were dispensed at 1,788 cells/well using a Multidrop Combi Reagent Dispenser (Thermo Scientific) into white 96-well plates pre-dispensed with antibody dilutions. A two-fold dilution series was prepared for each antibody in sterile polypropylene 96-well plates. Assay plates were covered with Microclime Environmental Lids (Beckman Coulter 0015717) filled with 8 mL of UltraPure Distilled water (Invitrogen 10977-015) to reduce well evaporation. Cell proliferation was measured using the CellTiter-Glo 2.0 assay (Promega G9242) after 13 days of antibody treatment. Luminescence measurements were taken on an EnVision 2105 Multimode Plate Reader, and data were analyzed in GraphPad Prism using a variable slope (four parameters) fit with 1/Y² data weighting. Edge wells were excluded from analysis. Vectorized anti-HER2 antibodies of HER-04, HER-10, and HER15 inhibited cell growth of MDA-MB-361 cells in a dose-dependent manner (FIGS. 4A-4D).

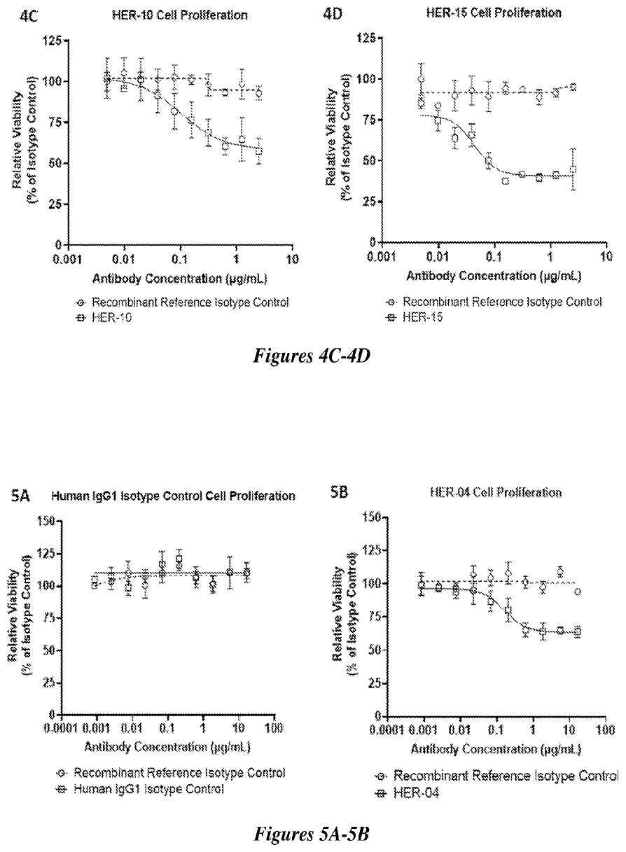

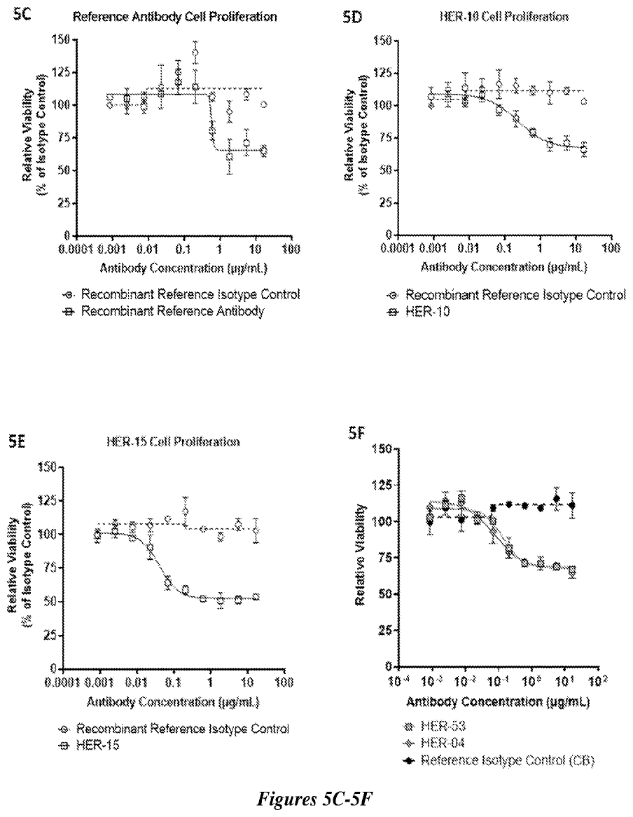

The above MDA-MB-361-luc cell proliferation assay was repeated using an expanded dose range utilizing a three-fold dilution series of each antibody. The vectorized anti-HER2 antibodies of HER-04, HER-10, HER-15, and HER-53 inhibited cell growth of MDA-MB-361-luc cells in a dose-dependent manner, while human IgG1 isotype control antibody did not (FIGS. 5A-5G, Tables 50 and 51). A similar result was found using high passage cells, p37, for the vectorized HER-10 (FIGS. 6A-6B), which inhibited cell growth of MDA-MB-361-luc cells in a dose-dependent manner.

TABLE 50
IC₅₀ and Eₘₐₓ HER-53 and HER-04 in MDA-MB-361 cells

| Antibody | IC₅₀ (μg/mL) | Eₘₐₓ (%) |
|---|---|---|
| HER-53 | 0.136 | 40.3 |
| HER-04 | 0.077 | 46.5 |

TABLE 51
IC₅₀ and Eₘₐₓ HER-53 and HER-10 in MDA-MB-361 cells

| Antibody | IC₅₀ (μg/mL) | Eₘₐₓ (%) |
|---|---|---|
| HER-53 | 0.162 | 45.5 |
| HER-10 | 0.048 | 56.5 |

### Antibody-Dependent Cellular Cytotoxicity (ADCC)

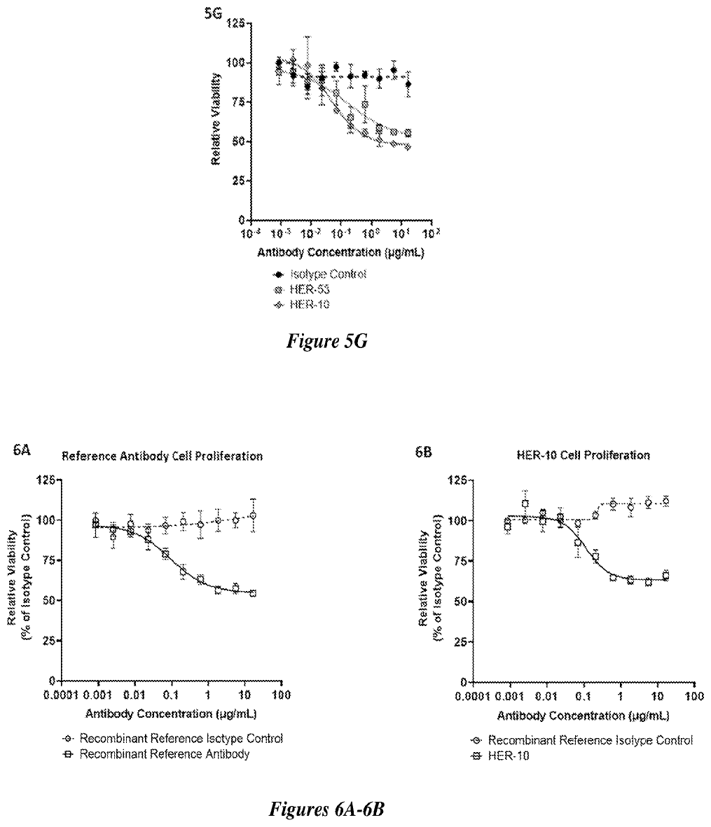

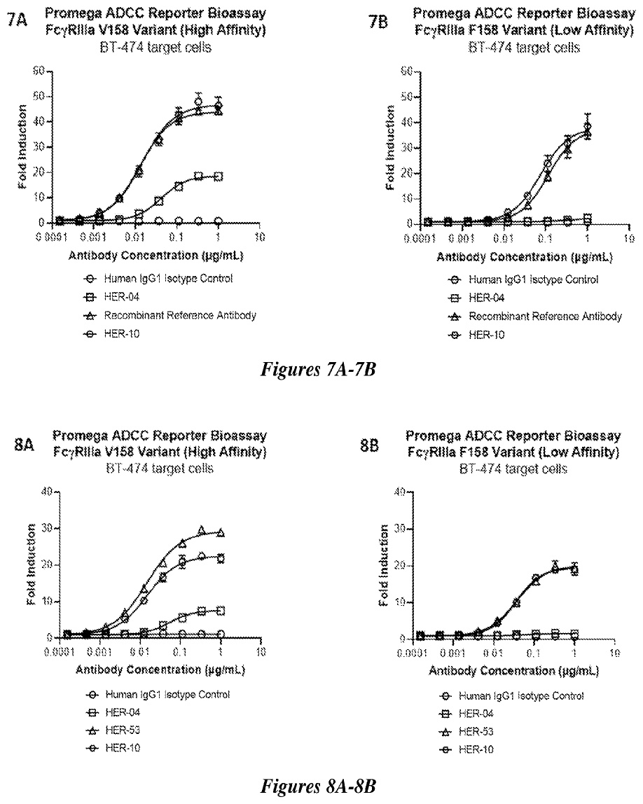

The ADCC Reporter Bioassays were run according to manufacturer protocols (Promega G7010/G9790). Briefly, BT-474 target cells were plated in white 96-well plates at 5,000 cells/well and incubated overnight at 37° C. and 5% CO₂. After overnight incubation, the media was replaced with ADCC Assay Buffer, and antibody dilutions were added in a 3-fold serial dilution series. Effector cells were immediately thawed and added to the plates for an Effector:Target cell ratio of 15:1. The plates were incubated at 37° C. and 5% CO₂ for 6 hours. The plates were then equilibrated to room temperature for 15 minutes and Bio-Glo Luciferase Assay Reagent was added. The plates were incubated for 30 minutes at room temperature, and luminescence was measured on a PerkinElmer EnVision 2105 Multimode Plate Reader. Data were analyzed on GraphPad Prism using a variable slope (four parameters) fit with 1/Y² data weighting. HER-10 contains enhancements in the Fc region that promote ADCC activity. The HER-10 ADCC activity was found to be similar to the reference antibody, and was improved over HER-04 in assays employing both the high affinity (FIG. 7A, Table 52) and low affinity variants of the FcgRIIIa receptor (FIG. 7B, Table 53). The Fc enhancements of HER-10 are further highlighted in the low affinity variant assay. HER-53 contains the same Fc region enhancements as HER-10, and ADCC activity is similar between the two antibodies. Both are improved over HER-04 in assays employing both the high affinity (FIG. 8A, Table 54) and low affinity variants of the FcgRIIIa receptor (FIG. 8B, Table 55).

TABLE 52
HER-10 and HER-04 EC₅₀ in BT-474 cells, High Affinity

| Antibody | Eₘₐₓ (ng/mL) |
|---|---|
| HER-10 | 14.8 |
| HER-04 | 42.2 |
| Reference Antibody | 12.9 |

TABLE 53
HER-10 EC₅₀ in BT-474 cells, Low Affinity

| Antibody | Eₘₐₓ (ng/mL) |
|---|---|
| HER-10 | 75.2 |
| Reference Antibody | 114.6 |

TABLE 54
HER-53, HER-10 and HER-04
EC₅₀ in BT-474 cells, High Affinity

| Antibody | Eₘₐₓ (ng/mL) |
|---|---|
| HER-53 | 14.5 |
| HER-10 | 14.8 |
| HER-04 | 52.6 |

TABLE 55
HER-53 and HER-10 EC₅₀ in BT-474 cells, Low Affinity

| Antibody | Eₘₐₓ (ng/mL) |
|---|---|
| HER-53 | 37.1 |
| HER-10 | 35.6 |

Cell Proliferation Assays with Tucatinib

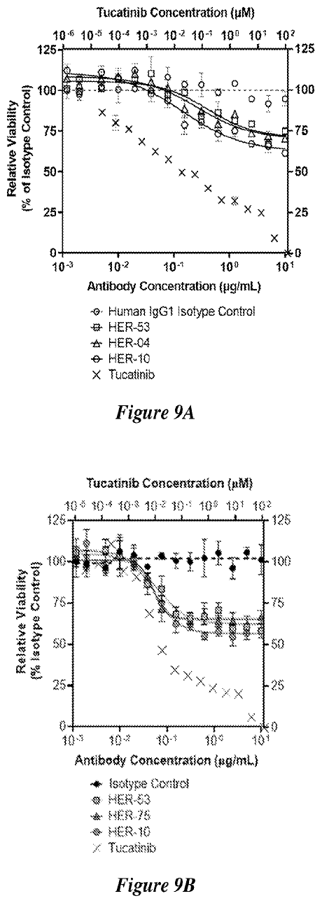

BT-474 cells were dispensed at 625 cells/well using a Multidrop Combi Reagent Dispenser (Thermo Scientific) into white 384-well plates pre-dispensed with antibody serial dilutions (two-fold) of human IgG1 isotype control antibody, HER-04, HER-10, HER-53, and HER-75 or tucatinib (an antineoplastic HER2 inhibitor) serial dilutions (three-fold). Assay plates were covered with Microclime Environmental Lids (Beckman Coulter 0015717) filled with 8 mL of UltraPure Distilled water (Invitrogen 10977-015) to reduce well evaporation. Cell proliferation was measured using the CellTiter-Glo 2.0 assay (Promega G9242) after four days. Luminescence measurements were taken on an EnVision 2105 Multimode Plate Reader, and data were analyzed in GraphPad Prism using a variable slope (four parameters) fit with 1/Y² data weighting. Edge wells were excluded from analysis. Similar cell growth inhibition was observed between the HER-53 antibody with an enhanced Fc region and HER-04 (FIG. 9A). A comparison between HER-53 and HER-75 is shown in FIG. 9B (see Table 56). Again, similar cell growth inhibition was observed between the HER-53 antibody and HER-75. A similar procedure was followed for testing with SK-BR-3 cells (FIG. 9C, Table 57). Further comparisons were made between HER-08 (control), HER-53, HER-75, and HER-10 utilizing the SK-BR-3 cell line (FIG. 9D, Table 56). HER-10 performed better than HER-53 or HER-04 in BT-474 cells. Tucatinib-treated cells served as positive control for cell growth inhibition.

TABLE 56
IC₅₀ and Eₘₐₓ of HER-53, HER-75, and HER-
10 in BT-474 cells and SK-BR-3 cells

| | BT-474 cells | | SK-BR-3 cells | |
|---|---|---|---|---|
| Antibody | IC₅₀ (μg/mL) | Eₘₐₓ (%) | IC₅₀ (μg/mL) | Eₘₐₓ (%) |
| HER-53 | 0.063 | 42.8 | 0.056 | 39.1 |
| HER-75 | 0.051 | 35.6 | 0.054 | 31.8 |
| HER-10 | 0.054 | 50.5 | 0.055 | 41.0 |

TABLE 57
IC₅₀ and Eₘₐₓ of HER-53, HER-04, and HER-10 in SK-BR-3 cells

| Antibody | IC₅₀ (μg/mL) | Eₘₐₓ (%) |
|---|---|---|
| HER-53 | 0.072 | 41.6 |
| HER-04 | 0.087 | 40.7 |
| HER-10 | 0.063 | 44.6 |

### BT-474 Cell Proliferation Assays—FcRn-Binding Mutants

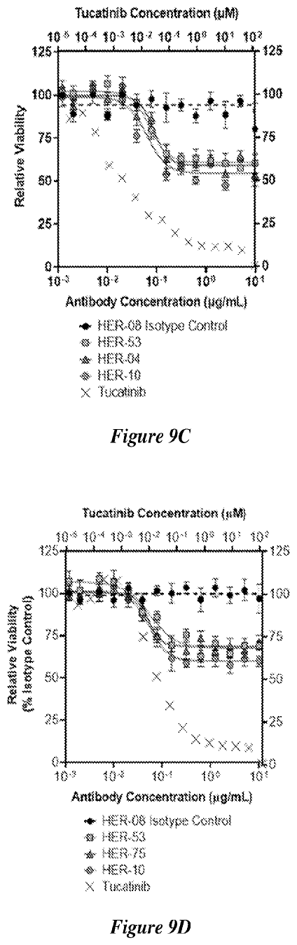

BT-474 cells were dispensed at 625 cells/well using a Multidrop Combi Reagent Dispenser (Thermo Scientific) into white 384-well plates pre-dispensed with antibody dilutions. A two-fold serial dilution series was prepared for each antibody (HER-04, human IgG1 isotype control antibody, HER-43, HER-46, and HER-47) in sterile polypropylene 96-well plates. Assay plates were covered with Microclime Environmental Lids (Beckman Coulter 0015717) filled with 8 mL of UltraPure Distilled water (Invitrogen 10977-015) to reduce well evaporation. Cell proliferation was measured using the CellTiter-Glo 2.0 assay (Promega G9242) after four days of antibody treatment. Luminescence measurements were taken on an EnVision 2105 Multimode Plate Reader, and data were analyzed in GraphPad Prism using a variable slope (four parameters) fit with 1/Y² data weighting. Edge wells were excluded from analysis. A similar cell growth inhibition was observed between HER-04 and engineered antibodies containing mutations that abrogate FcRn-binding (HER-43, HER-46, and HER-47) (FIGS. 10A-10B).

### SK-BR-3 and BT-474 Cell Proliferation Assays of HER-57

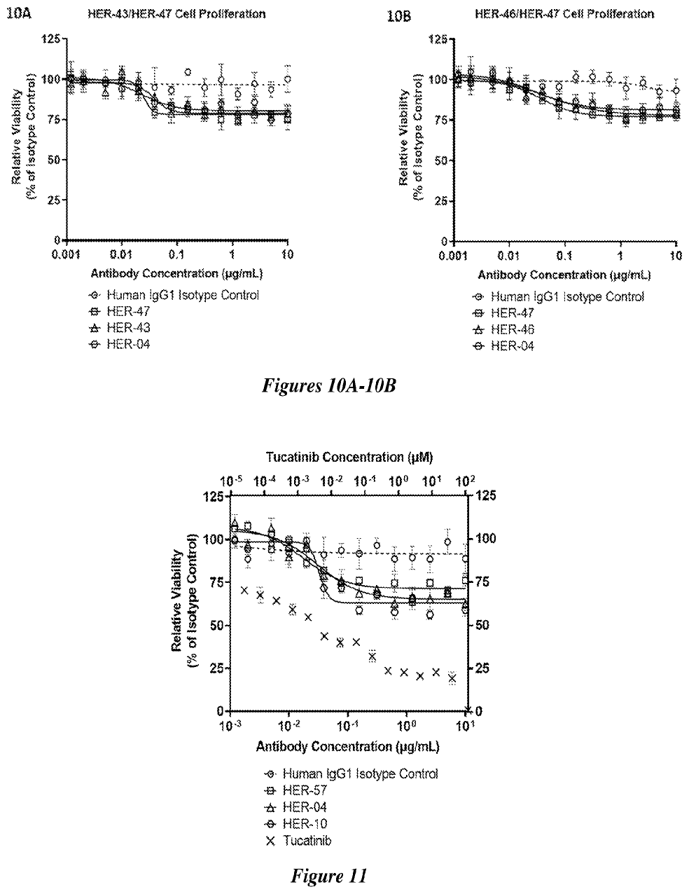

SK-BR-3 cells were dispensed at 625 cells/well using a Multidrop Combi Reagent Dispenser (Thermo Scientific) into white 384-well plates pre-dispensed with antibody serial dilutions (two-fold) or tucatinib serial dilutions (three-fold). Assay plates were covered with Microclime Environmental Lids (Beckman Coulter 0015717) filled with 8 mL of UltraPure Distilled water (Invitrogen 10977-015) to reduce well evaporation. Cell proliferation was measured using the CellTiter-Glo 2.0 assay (Promega G9242) after four days. Luminescence measurements were taken on an EnVision 2105 Multimode Plate Reader, and data were analyzed in GraphPad Prism using a variable slope (four parameters) fit with 1/Y² data weighting (FIG. 11). Edge wells were excluded from analysis. SK-BR-3 cells were sensitive to cell growth inhibition by the HER-57 antibody. HER-57, HER-04, and HER-10 all produced a similar cell growth inhibition, with HER-10 having a slightly larger maximal inhibition. Tucatinib-treated cells served as positive control for cell growth inhibition. A similar procedure was followed for testing with BT-474 cells. In contrast to SK-BR-3 cells, BT-474 cells were not sensitive to cell growth inhibition by HER-57.

### SK-BR-3 and BT-474 Cell Proliferation Assays of Heavy CDR3 Mutants

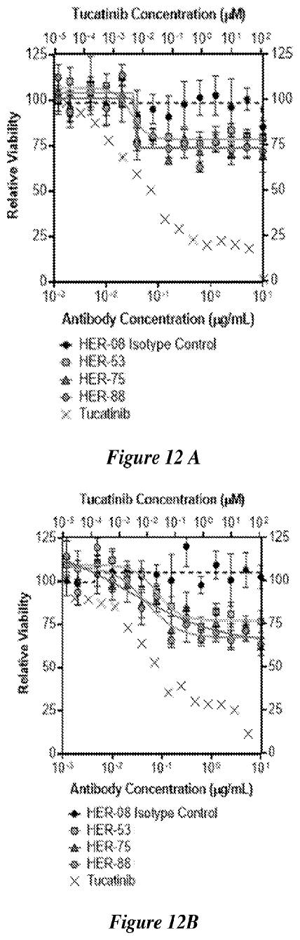

HER-08 (control), HER-53, HER-75, and HER-88, were analyzed by cell proliferation assays using SK-BR-3 cells, and BT-47 cells as described above. HER-53, HER-75, and HER-88 all produced cell growth inhibition in both SK-BR-3 cells (FIG. 12A and Table 59) and BT-474 cells (FIG. 12B and Table 60). Tucatinib-treated cells served as positive control for cell growth inhibition.

TABLE 59
IC₅₀ and Eₘₐₓ of HER-53, HER-75, and HER-88 in SK-BR-3 cells

| Antibody | IC₅₀ (μg/mL) | Eₘₐₓ (%) |
|---|---|---|
| HER-53 | 0.031 | 28.2 |
| HER-75 | 0.037 | 27.3 |
| HER-88 | 0.036 | 30.3 |

TABLE 60
IC₅₀ and Eₘₐₓ HER-53 and HER-88 in BT-474 cells

| Antibody | IC₅₀ (μg/mL) | Eₘₐₓ (%) |
|---|---|---|
| HER-53 | 0.081 | 32.4 |
| HER-88 | 0.048 | 41.5 |

Similar testing was done on HER-88, HER-89, HER-90, HER-91, and HER-92. Cell proliferation was measured using the CellTiter-Glo 2.0 assay (Promega G9242) after five days in the BT-474 cell line. Four replicate 384-well plates with 3 wells per point were used. These data are described in Table 48. HER-75 was used as a control to see the effect the CDR3 mutations had on cell proliferation. Similar results were found for HER-75, HER-88, HER-89, HER-90, HER-91, and HER-92.

TABLE 48
Cell Proliferation with HCDR3 mutants

| | IC₅₀ (μg/mL) | | | | | |
|---|---|---|---|---|---|---|
| Antibody | Plate 1 | Plate 2 | Plate 3 | Plate 4 | Average | StDev |
| HER-75 D98, M100, D101, | 0.038 | 0.055 | 0.045 | 0.060 | 0.050 | 0.010 |
| Y102L | | | | | | |
| HER-88 D98W, M100F, D101A, | 0.049 | 0.057 | 0.053 | 0.070 | 0.057 | 0.009 |
| Y102L | | | | | | |
| HER-89 D98W, M100F, D101A | 0.056 | 0.063 | 0.056 | 0.049 | 0.056 | 0.006 |
| HER-90 D98W, M100F, Y102L | 0.058 | 0.052 | 0.069 | 0.067 | 0.062 | 0.008 |
| HER-91 D98W, D101A, Y102L | 0.050 | 0.049 | 0.056 | 0.053 | 0.052 | 0.003 |
| HER-92 M100F, D101A, Y102L | 0.038 | 0.037 | 0.037 | 0.043 | 0.038 | 0.003 |
| | Eₘₐₓ (%) | | | | | |
| Antibody | Plate 1 | Plate 2 | Plate 3 | Plate 4 | Average | StDev |
| HER-75 D98, M100, D101, | 30.6 | 33.2 | 35.0 | 33.4 | 33.1 | 1.8 |
| Y102L | | | | | | |
| HER-88 D98W, M100F, D101A, | 41.3 | 39.4 | 40.4 | 38.4 | 39.9 | 1.3 |
| Y102L | | | | | | |
| HER-89 D98W, M100F, D101A | 42.0 | 39.8 | 45.4 | 43.4 | 42.7 | 2.4 |
| HER-90 D98W, M100F, Y102L | 41.4 | 40.1 | 41.0 | 41.6 | 41.0 | 0.7 |
| HER-91 D98W, D101A, Y102L | 48.1 | 39.8 | 46.3 | 41.2 | 43.8 | 4.0 |
| HER-92 M100F, D101A, Y102L | 37.0 | 35.1 | 37.5 | 34.6 | 36.1 | 1.4 |

### Example 5. In Vivo Characterization of Anti-HER2 Monospecific Antibody Molecules

### Quantification In Vivo in Mouse Serum and Brain Homogenates

HER-10 vectorized in VOY9P39 capsid (5.0E11 VG/mouse) or vectorized human IgG1 isotype control antibody were prophylactically administered IV to Fox Chase SCID CB17 mice (Charles River Labs, #236) 12 days prior to xenotransplantation of MDA-MB-361-luc tumor cells in the brain. Mouse serum and brain homogenate samples were collected 30 days post-xenograft and analyzed for functional antibodies by quantifying the percentage of total human IgG1 that binds to HER2 in a custom AlphaLISA assay. To quantify HER2 binding, 10 μL of samples (1:1,000 serum dilution or 1:50 brain homogenate dilution) were added to white 384-well plates and were mixed with recombinant his-tagged HER2 ECD (R&D Systems), Protein A Alpha Donor Beads (Perkin Elmer), and Nickel Chelate AlphaLISA Acceptor Beads (Perkin Elmer) in a 40 uL final assay volume. Functional binding was interpolated from a standard curve of a reference anti-HER2 antibody. Total human IgG1 was quantified via an AlphaLISA Detection Kit (Perkin Elmer AL307) following manufacturer protocols. Briefly, for total human IgG1 detection, 5 μL of sample was added to a white 384-well plate and incubated with AlphaLISA beads in a 50 uL final assay volume. AlphaLISA readings were taken on a PerkinElmer EnVision 2105 Multimode Plate Reader. Standard curves and data interpolation were analyzed in GraphPad Prism using a sigmoidal 4PL fit with 1/Y² data weighting. The HER-10 antibodies in mouse serum were found to be ˜89% functional when quantified via a HER2-binding AlphaLISA assay and normalized against total human IgG1. The human IgG1 isotype control antibodies displayed negligible HER2 binding. Percentages of functional antibody from brain tissue homogenate could not be calculated since samples fell outside the linear range of the assay.

### Example 6. In Vivo Characterization of Vectorized Anti-HER2 Monospecific Antibody Molecules

The brain distribution of various capsids targeting the central nervous system (CNS) in mice (VOY101, VOY9P39, and VOY9P33) was studied to determine which capsid to use for vectorization of the anti-HER2 constructs. VOY9P39 and VOY9P33, are capsids created by the TRACER method as described in WO2020/072683, Nonnenmacher et al. 2019 Molecular Therapy 27(4S1):27-27, and Nonnenmacher et al. 2021 Molecular Therapy—Methods & Clinical Development 20:366-378, which are hereby incorporated by reference in their entirety.

Capsid were compared for their brain distributions after IV administration of 4e11 VG/100 uL/Mouse IV injections of CNS targeting capsids in CB17/SCID mice (BALB/C background) with CBA-Driven EGFP transgene.

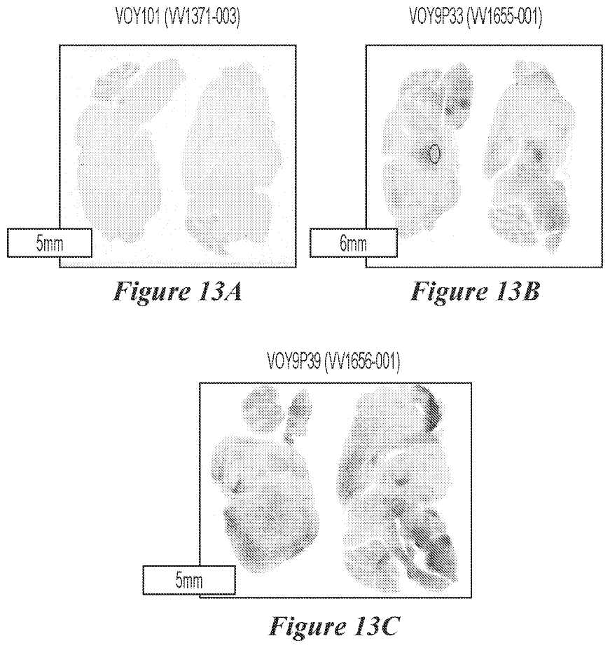

Orthotopic xenografts of BT474 passage 24 cells were injected (250,000 cells/2 uL/mouse) intracranially into female Fox Chase SCID CB17 (Mutation: Icr-Prkdcscid/IcrIcoCrl) congenic immunodeficient mice from Charles River Laboratories (#3611), that were 65 days old. The injections were 2.5 mm (lateral), −1 mm (posterior) with respect to bregma, lowered −3 mm ventral and raised +0.5 mm dorsal to a final −2.5 mm ventral position. 6 days later, dilutions of VOY101 or the CNS targeting capsids VOY9P33 or VOY9P39 mediating GFP transgene expression underwent IV injection of 100 uL (4e11 VG/animal), administered through the tail veins of mice, n=5 mice per cohort. At 7 days post AAV, the respective 5 mouse cohorts were euthanized and underwent necropsy. Immunohistochemistry was performed and sagittal sections were stained for anti GFP (for AAV expression) and anti human nucleoli (for human tumor cell identification). FIGS. 13A-13C show the brain distributions of the tested capsids.

### PK/PD Dynamics in the Serum, CSF, and Brain Post Intravenous Administration of HER-10 Vectorized in VOY9P39 Capsid

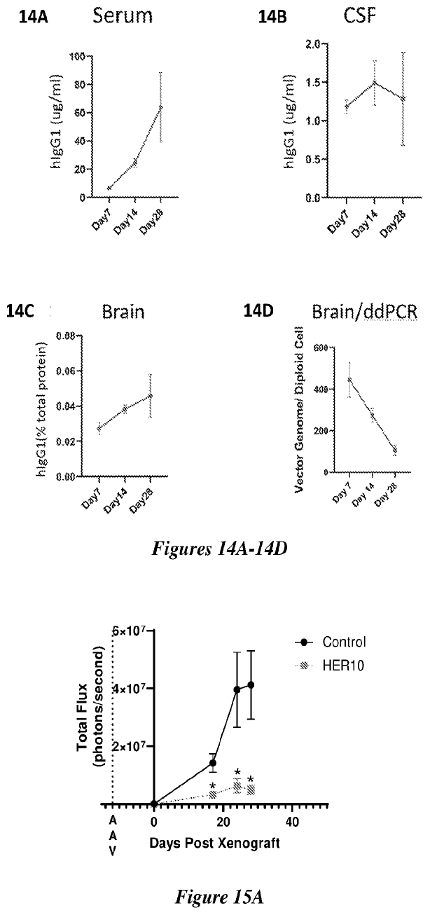

Orthotopic xenografts of MDA-MB-361-Luc #1 passage 33 cells grown as tumorspheres (in tumorsphere media; Sigma #C-28070) were injected (250,000 cells/2 uL/mouse) intracranially into female Fox Chase SCID CB17 (Mutation: Icr-Prkdcscid/IcrIcoCrl) congenic immunodeficient mice from Charles River Laboratories (#3611), that were 60 days old. The injections were 2.5 mm (lateral), −1 mm (posterior) with respect to bregma, lowered −3 mm ventral and raised +0.5 mm dorsal to a final −2.5 mm ventral position. 9 days later, dilutions of the AAV comprised of a CNS targeting novel capsid VOY9P39 and a genome encoding HER-10 was prepared. IV injections of 100 uL (5e11 VG/animal) were administered through the tail veins of mice, n=3 mice per cohort. At 7, 14, and 28 days post AAV, the respective 3 mouse cohorts were euthanized and underwent necropsy. AlphaLISA quantification of hIgG1 was performed on the serum, CSF and brain (contralateral to xenograft injection site) samples, ddPCR was performed on cryopulverized brain samples, contralateral to xenograft injection site. Table 32 shows the concentration of the antibodies from different locations and at varying time frames from AAV treatment. FIGS. 14A-14D show the concentration of the HER-10 antibody in the serum, CSF, brain tissue, and brain/ddPCR on days 7, 14, and 28 post AAV treatment.

TABLE 32
Antibody Concentration after treatment with HER10
(5e11 VG/mouse; N = 3) at day 9 post-xenograft

| Source | Day Post AAV | Mean | Units | Std. Dev. | Std. Error |
|---|---|---|---|---|---|
| Serum | 7 | 6.617 | (ug/ml) | 1.19 | 0.6869 |
| | 14 | 24.6 | (ug/ml) | 4.997 | 2.885 |
| | 28 | 63.86 | (ug/ml) | 42.69 | 24.64 |
| CSF | 7 | 1.182 | (ug/ml) | 0.1563 | 0.09025 |
| | 14 | 1.492 | (ug/ml) | 0.502 | 0.2898 |
| | 28 | 1.286 | (ug/ml) | 0.8529 | 0.6031 |
| Brain | 7 | 0.02724 | % total protein | 0.00548 | 0.003164 |
| | 14 | 0.03833 | % total protein | 0.003843 | 0.002219 |
| | 28 | 0.04583 | % total protein | 0.02092 | 0.01208 |
| Brain/ddPCR | 7 | 445 | vector genome/diploid cell | 142.2 | 82.08 |
| | 14 | 274.3 | vector genome/diploid cell | 57.06 | 32.95 |
| | 28 | 105 | vector genome/diploid cell | 43.58 | 25.16 |

### Prophylactic Treatment Using IV Administration of HER-10 Vectorized in VOY9P39 Capsid (5e11 VG/Mouse) of Prior to Xenograft

Dilutions of the AAV particles composed of a CNS targeting capsid VOY9P39 and a genome encoding either human IgG1 isotype control antibody) or HER-10 were prepared in a blinded manner to the study experimenters. IV injections of 100 uL (5e11 VG/animal) were administered through the tail veins of mice, n=10 mice per cohort. Twelve days later, orthotopic xenografts of MDA-MB-361-Luc #1 passage 33 cells grown as tumorspheres (in tumorsphere media; Sigma #C-28070) were injected intracranially (250,000 cells/2 uL/mouse). The mice used were female, Fox Chase SCID CB17 Mutation: Ter-Prkdcscid/IcrIeoCrl Congenic Immunodeficient from Charles River Laboratories (#3611); 88 days old. The injections were 2.5 mm (lateral), −1 mm (posterior) with respect to bregma, lowered −3 mm ventral and raised +0.5 mm dorsal to a final −2.5 mm ventral position. 7 Days later and every week after that until day 28 post xenograft, mice were imaged in an AmiHTX (Spectral Imager) for bioluminescence of the human tumor cells expression of luciferase in response to intraperitoneal luciferin injections. At day 30, the mice were euthanized and necropsies performed. Immunohistochemistry staining for H&E, anti-GFP, and anti-human nucleoli was performed. AlphaLISA quantification of hIgG1 was performed and normalized to a % total protein in the case of brain homogenate, ddPCR quantification of total vector genomes per diploid cell was performed on cryopulverized brain samples. Table 33 displays the data from the vectorized HER-10 treatment in comparison to the vectorized human IgG1 isotype control antibody.

TABLE 33
iv administration of HER-10 vectorized in VOY9P39
capsid (5e11 VG/mouse, 42 Days Post AAV, N = 9)

| | Treatment | | | | Std. | |
|---|---|---|---|---|---|---|
| Source | Type | Treatment | Mean | Units | Dev. | Std. Error |
| Serum | Day 12 | Ctrl (HER08) | 39.61 | (ug/ml) | 11.94 | 3.981 |
| | Prophylactic | HER10 | 102.1 | (ug/ml) | 35.89 | 11.96 |
| Brain | | Ctrl (HER08) | 0.02035 | % total protein | 0.008788 | 0.002929 |
| | | HER10 | 0.04654 | % total protein | 0.01547 | 0.005156 |
| Brain/ddPCR | | Ctrl (HER08) | 68.29 | vector | 19.52 | 6.506 |
| | | | | genomes/diploid | | |
| | | | | cell | | |
| | | HER10 | 75.11 | vector | 24.92 | 8.306 |
| | | | | genomes/diploid | | |
| | | | | cell | | |

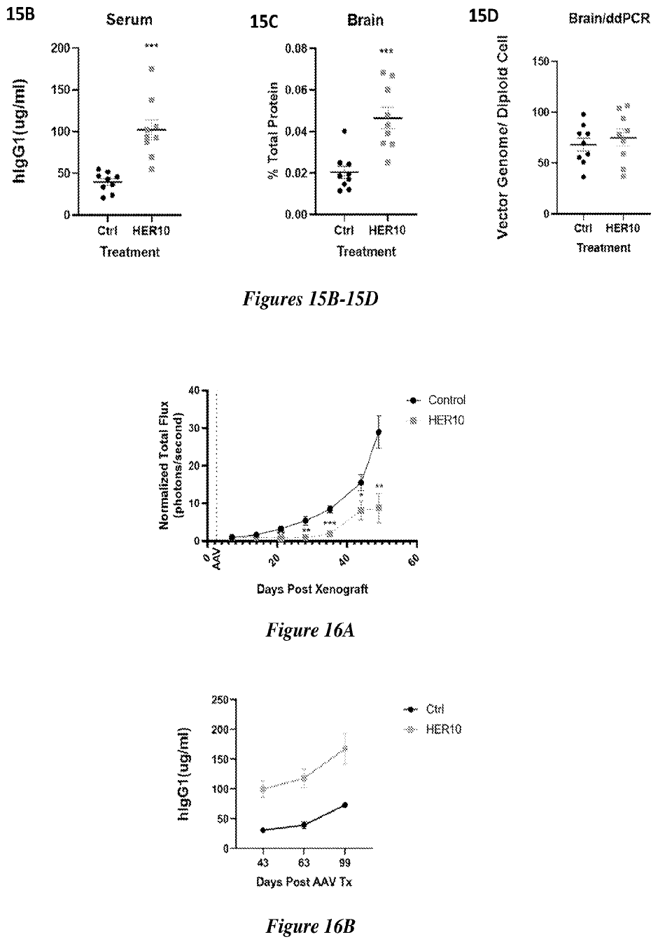

Prophylactic treatment 12 days prior to xenograft with IV administration of HER-10 delivered by the CNS targeting capsid VOY9P39 (5e11 VG/mouse) demonstrated persistent and significant abrogation of tumor growth as compared to control (hIgG1) treated mice. FIGS. 15A-15D show the comparison of the control to the HER-10 in the in the serum, CSF, brain tissue, and brain ddPCR.

IV Treatment with HER-10 or HER-53 Vectorized in VOY9P39 Capsid (2.5e11 VG/Mouse) on Day 2 Post-Xenograft

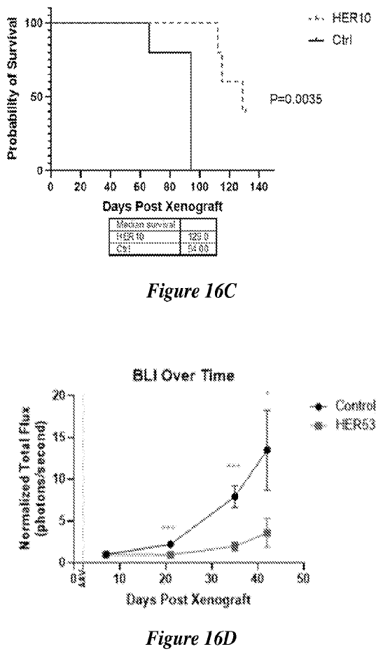

Orthotopic xenografts of MDA-MB-361-Luc #1 passage 33 cells grown as tumorspheres (in tumorsphere media; Sigma #C-28070) were injected (125,000 cells/2 uL/mouse) intracranially into female Fox Chase SCID CB17 (Mutation: Icr-Prkdcscid/IcrIcoCrl) congenic immunodeficient mice from Charles River Laboratories (#3611), that were 60 days old. The injections were 2.5 mm (lateral), −1 mm (posterior) with respect to bregma, lowered −3 mm ventral and raised +0.5 mm dorsal to a final −2.5 mm ventral position. Two days later, dilutions of AAV particles composed of the CNS targeting capsid VOY9P39 and a genome encoding either human IgG1 isotype control antibody, HER-10 or HER-53 were prepared in a blinded manner to the study experimenters. IV injections of 100 uL (2.5E11 vector genomes per animal) were administered through the tail veins of mice, n=5 mice per cohort. 7 Days later and every week after that until day 49 post xenograft, mice were imaged in an AmiHTX (Spectral Imager) for bioluminescence of the human tumor cells expression of luciferase in response to intraperitoneal luciferin injections. Blood collection was performed with a syringe inserted into tail vein at days 43 and 63 post AAV injections (Table 44). AlphaLISA quantification of hIgG1 was performed on the serum samples. Survival of mice was monitored with twice weekly weighing and symptoms of tumor burden (pain, hunched, ruffled fur, seizures, motor impairment, or −20% weight loss) resulted in euthanasia and removal from study. FIGS. 16A-16E show the normalized total flux, the concentration of the antibody and probability of survival for the HER-10 (FIGS. 16A-16C) and HER-53 (FIGS. 16D-16E) iv treatments.

TABLE 44
iv administration of HER-10 vectorized in VOY9P39 capsid (2.5e11
VG/mouse, Treatment Day 2 Post Xerograph, Serum Source)

| Day | | | | | Std. | Std. |
|---|---|---|---|---|---|---|
| Post AAV | Treatment | n | Mean | Units | Dev. | Error |
| 43 | Ctrl (HER08) | 5 | 30.72 | (ug/ml) | 6.271 | 2.805 |
| 43 | HER10 | 5 | 99.64 | (ug/ml) | 30.82 | 13.78 |
| 63 | Ctrl (HER08) | 4 | 39.21 | (ug/ml) | 11.73 | 5.866 |
| 63 | HER10 | 5 | 118.1 | (ug/ml) | 34.75 | 15.54 |
| 99 | Ctrl (HER08) | 2 | 67.64 | (ug/ml) | 9.43 | 5.44 |
| 99 | HER10 | 5 | 162.3 | (ug/ml) | 47.1 | 14.9 |

Two days post xenograft IV treatment with HER-10 delivered by the CNS targeting AAV capsid VOY9P39 (2.5E11 vector genomes per mouse) demonstrated persistent and significant tumor growth suppression as compared to human IgG1 (Control_hIgG1) treated mice.

Intracranial Treatment with HER-10 Vectorized in VOY-101 Capsid (6e10 VG/Mouse) on Day 2 Post-Xenograft

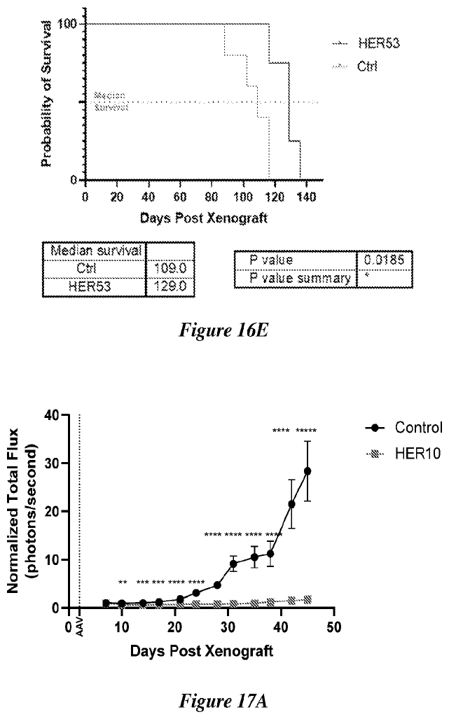

Orthotopic xenografts of BT474 cells were injected (250,000 cells/2 uL/mouse) intracranially into female Fox Chase SCID CB17 (Mutation: Icr-Prkdcscid/IcrIcoCrl) congenic immunodeficient mice from Charles River Laboratories (#3611), that were 60 days old. The injections were 2.5 mm (lateral), −1 mm (posterior) with respect to bregma, lowered −3 mm ventral and raised +0.5 mm dorsal to a final −2.5 mm ventral position. A guide screw cannula was implanted into the injection site. Two days later, dilutions of AAV particles composed of the VOY-101 capsid and a genome encoding either HER-10 or a control (hIgG1-Ctrl) were prepared in a blinded manner to the study experimenters. An intracranial injection of 6e10 VG/animal was administered through the cannula into the striatum, n=15 mice per cohort. 7 Days after the xenograft, and twice weekly after that, mice were imaged in an IVIS for bioluminescence of the human tumor cells expression of luciferase in response to subcutaneous luciferin injections. Survival of mice was monitored with twice weekly weighing and symptoms of tumor burden (pain, hunched, ruffled fur, seizures, motor impairment, or −20% weight loss) resulted in euthanasia and removal from study. FIGS. 17A and 17B shows the normalized total flux and probability of survival, respectively, comparing the vectorized HER-10 and the control treatment.

Two days post xenograft intracranial treatment with HER10 delivered by VOY-101 (6e10 VG/mouse) demonstrates persistent and significant tumor growth suppression as compared to human IgG1 (Control_hIgG1) treated mice.

Innate Immune Response Upon Treatment with HER-10 Vectorized in AAV9P39-Capsid (2.5e11 VG/Mouse) as Evidenced by Increased in Overall CD45⁺ Cells

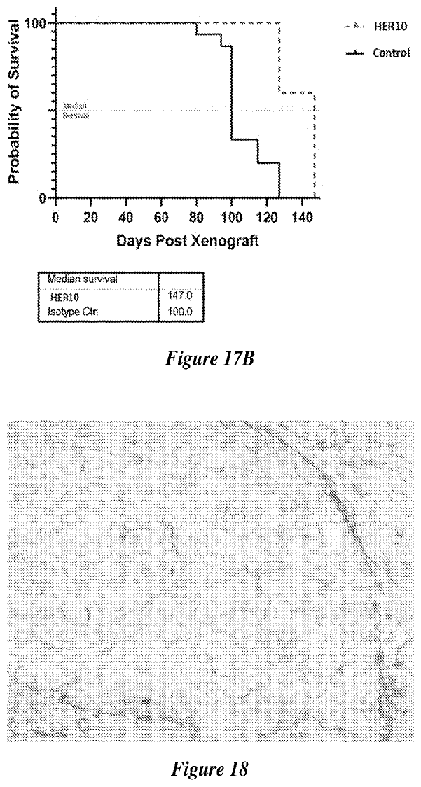

After Cd11b IHC staining of formalin fixed paraffin embedded sections from 28-day old orthotopic xenografts derived from transplantation of MDA-MB-361 cells into mouse brain and 2 day post treated with i.v. administration of AAV9P39-HER-10 (2.5e11 VG/mouse) evidence of innate immune cells surrounding and infiltrating the tumor was discovered (FIG. 18). To investigate this further single cell RNA sequencing was carried out on mouse immune cells (CD45t) derived from the peritumoral area from HER08-Ctrl and HER10 treated xenografts at day 28 post AAV9P39 treatment. Orthotopic xenografts of MDA-MB-361-Luc #1 passage 30 cells grown as tumorspheres (in tumorsphere media; Sigma #C-28070) were injected (250,000 cells/2 uL/mouse) intracranially into female Fox Chase SCID CB17 (Mutation: Icr-Prkdcscid/IcrIcoCrl) congenic immunodeficient mice from Charles River Laboratories (#3611), that were 62 days old. The injections were 2.5 mm (lateral), −1 mm (posterior) with respect to bregma, lowered −3 mm ventral and raised +0.5 mm dorsal to a final −2.5 mm ventral position. Two days later, dilutions of blood brain barrier penetrant novel capsid VOY9P39 mediating either HER-08 (hIgG1) or HER-10 were prepared. IV injections of 100 uL (2.5e11 VG/animal) were administered through the tail veins of mice, n=5 mice per cohort. 7 Days later mice were imaged in an AmiHTX (Spectral Imager) for bioluminescence of the human tumor cells expression of luciferase in response to intraperitoneal luciferin injections.

Two tumor bearing mice from each cohort (9P39, HER08-Ctrl vs Her10) were weighed and then injected with 10 ml/Kg of ketamine cocktail (ketamine, xylazine and acepromazine injectable drugs). After 5 minutes, and when mice were knocked out and unresponsive to toe pinch each mouse underwent trans-cardial perfusion, performed with PBS. The brains were removed and right hemi-brain area around tumor cell injection site was cut off in a brain matrice (Fisher Sci #50-195-4415) with a razor blade. The brains were minced in L15 and digested at 6 C for 25 minutes in protease inhibitor (Creative Biomart #NATE-0633) and dissociated. Myelin depletion was performed (Miltenyi, #130-096-731), and then cells were stained with CD45 antibody (Miltenyi, #130-110-796) and FACS sorted for 7AAD⁻ (viability) and CD45⁺ (immune cells). There was 1.5× higher proportion of CD45⁺ cells in the HER-10 treated cohort indicating an enrichment of the innate immune response in that condition. Cells were loaded on a 10× chromium G chip and the scRNA-Seq was run and processed according to manufacturer's protocols (10× Genomics). Samples were sequenced on a NextGen500 Sequencing machine (Illumina).

The resultant FASTQ files were demultiplexed and aligned to the MM10 reference genome using CellRanger (10× Genomics). Next, the resultant data matrix of cell counts was processed using RStudio and the Seurat package with R version 4.1.1. The data was filtered to include only cells with only greater than 1000 genes per cell and less than 5000, and less than 20 percent mitochondrial gene expression. The 2 data matrix files were normalized, scaled, and integrated into one combined dataset. Clusters were generated with a resolution of 0.3 and each cluster identity was determined using a panel of cell type specific genes (Hammond et al., Immunity, 50(1):253-271 (2019); Hove et al., Nature Neuroscience 22:1021-1035 (2019); Ochocka et al., Nat Commun 12:1151 (2021); Villani et al., Science 356(6335):eaah4573 (2017), which are hereby incorporated by reference in their entirety).

Proliferating microglia (MKI67 high expressing microglia cells) were enriched by almost three-fold as a proportion of total microglia in the HER10 treated mouse brain CD45⁺ cells as compared to the HER08-Ctrl treatment conditions (Table 45). Moreover, natural killer cells (NK), dendritic cells (DC), and innate lymphocytic cells (ILC), were more abundant as cellular populations determined by their proportion of total mouse brain CD45⁺ cells in the HER10 cohort as compared to the HER08-Ctrl cohort (Table 46).

TABLE 45
Microglia Cell Types as a Percent of Total Microglia by Treatment

| Microglia Cluster | | | Proportion |
|---|---|---|---|
| Identity | HER08-Ctrl | HER10 | HER10/HER08 |
| aMic | 12.30 | 16.90 | 1.37 |
| pMic | 1.40 | 4.00 | 2.86 |
| hMic | 87.67 | 83.12 | 0.95 |

aMic = active microglia (Clusters 3 & 9)
pMic = proliferation microglia (Cluster 9)
hMic = homeostatic microglia (Clusters 0, & 2)

TABLE 46
Fold Change in Proportion of Cluster Cells to Total
CD45 Sorted Mouse Cell Population by Treatment

| Cluster Identity | HER10/HER08 |
|---|---|
| migDC | 30.37 |
| pDC | 21.09 |
| cDC2 | 8.16 |
| ILC2 | 5.17 |
| NK cells | 2.99 |
| dividing microglia | 1.88 |
| non class MC | 1.64 |
| Active Microglia | 0.77 |
| Microglia_1 | 0.63 |
| Microglia_2 | 0.57 |
| Macrophages | 0.52 |

Microglia; cDC2 (conventional); Macrophages; NK; ncMC (non-classical monocytes); ILC2 (innate lymphoid cell); pDC (plasmacytoid); migDC (migratory); neutrophils

An innate immune response was evident in the HER10 population as evidenced by an increased in overall CD45⁺ cells during FACS analysis, as well as increased proportions of active proliferating microglia, DC, NK cells, and ILC. The intriguing aspect of increased DC populations is that they mediate the interaction between the innate immune response and the adaptive immune response and therefore, perhaps our AAV mediated gene therapy may elicit an even greater therapeutic response in patients with intact immune systems rather than in our preclinical mouse models of immune incompetent mice.

### Example 7. Generation of Anti-HER2 Bispecific Antibody Molecules

Genetic element constructs were designed for AAV delivery of anti-HER2 bispecific antibodies. The nucleotide sequences from 5′ ITR to 3′ ITR of the genetic element constructs that comprise a transgene encoding a bispecific HER2 antibody molecules are provided as HER-73 (SEQ ID NO: 5190), HER-78 (SEQ ID NO: 5375), HER-97 (SEQ ID NO: 6505), HER-98 (SEQ ID NO: 6506), HER-99 (SEQ ID NO: 6507), HER-100 (SEQ ID NO: 6508), and HER-101 (SEQ ID NO: 6509) herein. These construct are also summarized in Tables 6, 25, and 43.

The genetic element construct comprises a nucleic acid comprising a transgene encoding a bispecific antibody molecule that binds to HER2. The transgene was designed to comprise a nucleotide sequence encoding an antibody heavy chain signal sequence (SEQ ID NO:5157), a nucleotide sequence encoding an antibody heavy chain variable region (VH) (SEQ ID NO: 5261), a nucleotide sequence encoding antibody heavy chain constant region (SEQ ID NO: 5215 or 5219), a nucleotide sequence encoding a linker region comprising a T2A linker (SEQ ID NO: 1726), which was flanked by a furin cleavage site (SEQ ID NO: 1724) at the 5′ end of the T2A linker, a nucleotide sequence encoding an antibody heavy chain variable region (VH) (SEQ ID NO: 6552, 6537, 6540, 6543, 6546, or 6549), a nucleotide sequence encoding an antibody light chain variable region (VL) (SEQ ID NO: 5265), and a nucleotide sequence encoding an antibody light chain constant region (SEQ ID NO: 5217).

The encoded antibody of HER-73, HER-78, HER-97, HER-98, HER-99, HER-100, and HER-101 comprise a heavy chain signal sequence (SEQ ID NO:5158), an antibody heavy chain variable region (VH) (SEQ ID NO: 5262, 6536, 6539, 6542, 6545, or 6548), an antibody heavy chain constant region (SEQ ID NO: 5216 or 5220), an antibody light chain variable region (VL) (SEQ ID NO: 5267) and an antibody light chain constant region (SEQ ID NO: 5218).

### Example 8. Generation of ITR-ITR Constructs for the Expression of Anti-HER2 Bispecific Antibody Molecules

In designing the genetic element constructs for expression of bispecific HER2 antibody molecules (HER-57, HER-62, HER-73, HER-78, HER-97, HER-98, HER-99, HER-100, and HER-101), several promoters were selected. The ubiquitous CB promoter (SEQ ID NO: 6566) was used for HER-62 (SEQ ID NO: 5188), HER-73 (SEQ ID NO: 5189), HER-78 (SEQ ID NO: 5375), HER-97 (SEQ ID NO: 6505), HER-98 (SEQ ID NO: 6506), HER-99 (SEQ ID NO: 6507), HER-100 (SEQ ID NO: 6508), and HER-101 (SEQ ID NO: 6509). The ubiquitous CB promoter (SEQ ID NO: 6566) further comprising a CMV(IE) promoter (SEQ ID NO: 6594), was used for HER-57 (SEQ ID NO: 5187). All nine bispecific HER2 antibodies further comprised an ie exon 1 region (SEQ ID NO: 6573), an ie intron 1 region (SEQ ID NO: 6578), a second human beta-globin intron region (SEQ ID NO: 6580), and a human beta-globin exon region (SEQ ID NO: 6576).

The 5′ ITR (SEQ ID NO: 6559), the polyadenylation sequence (SEQ ID NO: 6590), and the 3′ ITR (SEQ ID NO: 6561) were the same across all HER-57, HER-62, HER-73, HER-78, HER-97, HER-98, HER-99, HER-100, and HER-101 genetic element constructs designed for the expression of anti-HER2 bispecific antibody molecules.

All nine genetic element constructs and their component parts are summarized in Tables 6, 19, 24, 25, and 43 above.

### Example 9. In Vitro Characterization of Anti-HER2 Bispecific Antibody Molecules

### BT-474 and SK-BR-3 Cell Proliferation Assays of HER-73 and HER-78

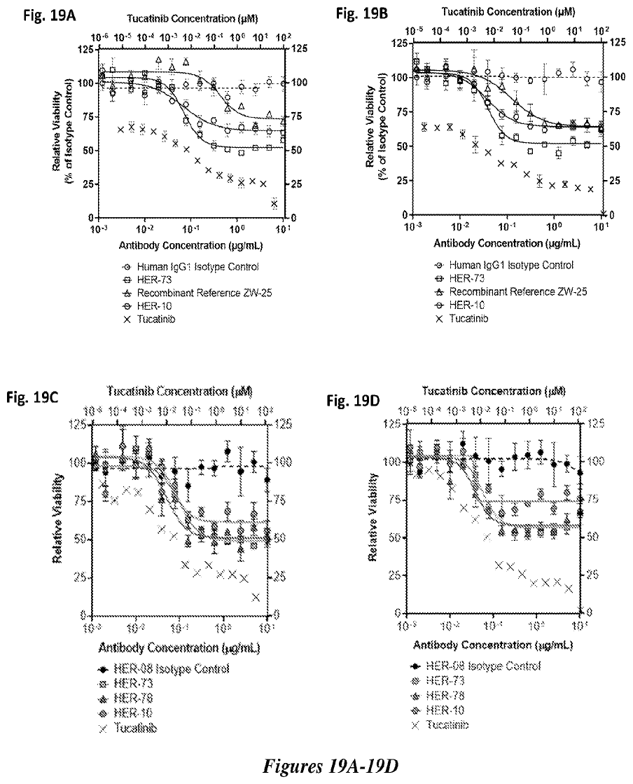

SK-BR-3 cells were dispensed at 625 cells/well using a Multidrop Combi Reagent Dispenser (Thermo Scientific) into white 384-well plates pre-dispensed with antibody serial dilutions (two-fold) or tucatinib serial dilutions (three-fold). The assay plates were covered with Microclime Environmental Lids (Beckman Coulter 0015717) filled with 8 mL of UltraPure Distilled water (Invitrogen 10977-015) to reduce well evaporation. Cell proliferation was measured using the CellTiter-Glo 2.0 assay (Promega G9242) after four days. Luminescence measurements were taken on an EnVision 2105 Multimode Plate Reader, and data were analyzed in GraphPad Prism using a variable slope (four parameters) fit with 1/Y² data weighting (FIGS. 19A and 19C, Tables 61 and 62). Edge wells were excluded from analysis. The HER-73 and HER-78 antibodies had a larger effect on cell growth inhibition of BT-474 cells than did HER-10 or the ZW-25 bispecific antibody. Tucatinib-treated cells served as positive control for cell growth inhibition. Similar to BT-474 cells, SK-BR-3 cells were sensitive to HER-73 and HER-78, which had a larger effect on cell growth inhibition than did HER-10 or the ZW-25 bispecific antibody (FIGS. 19B and 19D, Tables 61 and 62).

TABLE 61
IC₅₀ and Eₘₐₓ of HER-73, ZW-25, and HER-
10 in SK-BR-3 cells and BT-474 cells

| | SK-BR-3 cells | | BT-474 cells | |
|---|---|---|---|---|
| Antibody | IC₅₀ (μg/mL) | Eₘₐₓ (%) | IC₅₀ (μg/mL) | Eₘₐₓ (%) |
| HER-73 | 0.070 | 52.0 | 0.039 | 51.3 |
| ZW-25 | 0.431 | 35.0 | 0.125 | 41.5 |
| HER-10 | 0.068 | 36.6 | 0.038 | 40.0 |

TABLE 62
IC₅₀ and Eₘₐₓ of HER-73, HER-78, and HER-
10 in SK-BR-3 cells and BT-474 cells

| | SK-BR-3 cells | | BT-474 cells | |
|---|---|---|---|---|
| Antibody | IC₅₀ (μg/mL) | Eₘₐₓ (%) | IC₅₀ (μg/mL) | Eₘₐₓ (%) |
| HER-73 | 0.092 | 55.5 | 0.056 | 47.5 |
| HER-78 | 0.051 | 52.8 | 0.038 | 45.1 |
| HER-10 | 0.077 | 36.8 | 0.037 | 30.1 |

### Heavy Chain CDR3 Mutant Binding Affinity Measurements for Bispecific Antibody Molecules

The antibody binding of the heavy chain CDR3 mutants in a bispecific antibody molecule (HER-97, HER-98, HER-99, HER-100, HER-101) to HER2 was analyzed by Biacore 8K (GE Healthcare) surface plasmon resonance instrument (SPR) as described above and the antibody concentration range used was 0.625 to 40 nM (Table 73).

TABLE 73
Binding affinity of Heavy CDR3 mutants in a bispecific antibody to HER2

| | | | | | | | | Fit quality | |
|---|---|---|---|---|---|---|---|---|---|
| | | | | | | | | parameters | |
| | | | | | | | | | chi2/ |
| | HCDR3 | | | | | | | | Rmax |
| | Modif. | | Kinetic parameters | | | | | Kinetics | (%) |
| | (according | | KD | | | | Rmax | Chi² | (Calc. |
| Construct | to Kabat) | Replicate | (nM) | ka (1/Ms) | kd (1/s) | KD (M) | (RU) | (RU²) | unit) |
| HER78 | D98 | 1 | 0.54 | 1.38E+05 | 7.53E−05 | 5.44E−10 | 77.8 | 0.16 | 0.2% |
| | M100, | 2 | 0.57 | 1.74E+05 | 9.89E−05 | 5.70E−10 | 74.4 | 0.30 | 0.4% |
| | D101, | Mean | 0.44 | 1.64E+05 | 7.28E−05 | 4.36E−10 | 73.85 | | |
| | Y102 | % CV | 43.5 | 8.6 | 50.8 | 43.5 | 1.1 | | |
| HER97 | D98W, | 1 | 0.21 | 1.87E+05 | 3.98E−05 | 2.13E−10 | 71.4 | 0.25 | 0.4% |
| | M100F, | 2 | 0.27 | 1.84E+05 | 4.98E−05 | 2.70E−10 | 75.1 | 0.29 | 0.4% |
| | D101A, | Mean | 0.24 | 1.86E+05 | 4.48E−05 | 2.42E−10 | 73.25 | | |
| | Y102L | % CV | 16.7 | 1.1 | 15.8 | 16.7 | 3.6 | | |
| HER98 | D98W, | 1 | 0.29 | 1.47E+05 | 4.27E−05 | 2.90E−10 | 65.6 | 0.09 | 0.1% |
| | M100F, | 2 | 0.33 | 1.46E+05 | 4.82E−05 | 3.30E−10 | 67.5 | 0.11 | 0.2% |
| | D101A | Mean | 0.31 | 1.47E+05 | 4.55E−05 | 3.10E−10 | 66.55 | | |
| | | % CV | 9.1 | 0.5 | 8.6 | 9.1 | 2.0 | | |
| HER99 | D98W, | 1 | 0.33 | 1.79E+05 | 5.83E−05 | 3.25E−10 | 57.7 | 0.16 | 0.3% |
| | M100F, | 2 | 0.36 | 1.80E+05 | 6.42E−05 | 3.57E−10 | 59.7 | 0.15 | 0.2% |
| | Y102L | Mean | 0.34 | 1.80E+05 | 6.13E−05 | 3.41E−10 | 58.70 | | |
| | | % CV | 6.6 | 0.4 | 6.8 | 6.6 | 2.4 | | |
| HER100 | D98W, | 1 | 0.36 | 1.79E+05 | 6.46E−05 | 3.61E−10 | 49.6 | 0.08 | 0.2% |
| | D101A, | 2 | 0.37 | 1.82E+05 | 6.78E−05 | 3.72E−10 | 50.3 | 0.15 | 0.3% |
| | Y102L | Mean | 0.37 | 1.81E+05 | 6.62E−05 | 3.67E−10 | 49.95 | | |
| | | % CV | 2.1 | 1.2 | 3.4 | 2.1 | 1.0 | | |
| HER101 | M100F, | 1 | 0.27 | 1.72E+05 | 4.59E−05 | 2.67E−10 | 53.6 | 0.05 | 0.1% |
| | D101A, | 2 | 0.31 | 1.66E+05 | 5.10E−05 | 3.07E−10 | 55.7 | 0.06 | 0.1% |
| | Y102L | Mean | 0.29 | 1.69E+05 | 4.85E−05 | 2.87E−10 | 54.65 | | |
| | | % CV | 9.9 | 2.5 | 7.4 | 9.9 | 2.7 | | |

### SK-BR-3 Cell Proliferation Assays of Heavy Chain CDR3 Mutant for Bispecific Antibody Molecule (HER78)

SK-BR-3 cells were plated at 5000 cells/well into black 96-well plates with clear bottom. Next day, antibody dilutions (three-fold) were added to the plate such that the final concentration range of 0.001 to 10 pig/ml of antibody was achieved. Cell proliferation was measured using the CellTiter-Glo 2.0 assay (Promega G9242) after five days. Luminescence measurements were taken on SpectraMax M3 Reader, and data were analyzed in GraphiPad Prism using a variable slope (four parameters) fit with 1/Y² data weighting. Edge wells were excluded from analysis. Inhibition of cell proliferation was observed for all HCDR3 mutants of bispecific antibody but the IC50 values were higher than HER78. HER98 showed the lowest IC50 value among the mutants. HER08 was used as an isotype control in the experiment (Table 74).

TABLE 74
IC₅₀ and Eₘₐₓ of HER-78, HER-97, HER-98, HER-99,
HER-100 and HER-101 in SK-BR-3 cells

| Construct and HCDR3 Modifications | | IC50 | Emax |
|---|---|---|---|
| (according to Kabat) | | (μg/mL) | (%) |
| HER78 | D98 M100, D101, Y102 | 0.08 | 56.45 |
| HER97 | D98W, M100F, D101A, Y102L | 0.81 | 63.23 |
| HER98 | D98W, M100F, D101A | 0.19 | 52.02 |
| HER99 | D98W, M100F, Y102L | 0.87 | 49.23 |
| HER97 | D98W, D101A, Y102L | 0.66 | 59.11 |
| HER101 | M100F, D101A, Y102L | 0.60 | 61.03 |

### Example 10. High-Throughput Screen of TRACER AAV Library in NHP and Mice

A TRACER based method as described in WO2020072683, WO 2021/202651, and WO2021/230987, the contents of which are herein incorporated by reference in their entirety, was used to generate the AAV capsid variants described herein. An orthogonal evolution approach was combined with a high throughput screening by NGS. Briefly, the library of AAV capsid variants was generated using a sliding window approach, where 6 amino acid sequences were inserted into 8 different positions across loop IV of AAV9, including immediately subsequent to positions 453, 454, 455, 456, 457, 458, 459, and 460, relative to a reference sequence numbered according to SEQ ID NO: 138. The initial library was passed twice through non-human primates (NHP, 2-4 years of age). After the second passage (e.g., 28 days post injection into two NHPs), RNA was extracted from six brain regions. Following RNA recovery and RT-PCR amplification, a systematic NGS enrichment analysis was performed to calculate fold enrichment relative to an AAV9 wild-type control. Following these two passages, approximately 21195 variants were identified with an average fold change greater than wild-type. Of the 21195 variants, 1558 demonstrated a fold-change of greater than 6 compared to wild-type and were detected across all brain regions investigated. Of these 1558, approximately 1470 variants were selected for constructing a synthetic library and a third passage through two NHPs. Within the 1470 variants selected for further characterization and investigation, there was a relatively even distribution for each insertion position of the sliding window used to generate the initial library.

After creation of the synthetic library with the sub-selected variants, the synthetic library was screened (passage 3) in two NHPs (2-4 years of age) and two strains of mice, BALB/c (n=3, 6-8 weeks of age) and C57Bl/6 mice (n=3, 6-8 weeks of age), in a first cross-species evolution screen. The animals were injected intravenously with the synthetic library. After a period in vivo, (e.g., 28-days) RNA was extracted from nervous tissue, e.g., brain, spinal cord, and DRG of the NHPs and the brains of mice. Following RNA recovery and RT-PCR amplification, a systematic NGS enrichment analysis was performed, and the peptides comprised within the variants were identified and the capsid enrichment ratio for each variant compared to the wild-type AAV9 control was calculated (fold enrichment relative to wild-type AAV9) (Table 9). Values above 1 indicate an increase in expression relative to AAV9. All animals were dosed intravenously at 2-3 VG/kg across the screen.

As shown in Table 9, approximately 700 variants demonstrated an increase in expression relative to AAV9, and several variants demonstrated a greater than 10-fold enrichment relative to AAV9 in the brain of NHPs. Further, the variants demonstrating the greatest fold enrichment in the brain also demonstrated the greatest fold enrichment in the spinal cord relative to AAV9 in NHPs. These variants also demonstrated de-targeting in the DRG (data not shown). For instance, the variant comprising GSGSPHSKAQNQQT (SEQ ID NO: 200) demonstrated a 76.6-fold enrichment in the brain, a 29.4 fold enrichment in the spinal cord, and 0.4 fold enrichment in the DRG of NHPs relative to AAV9; and GHDSPHKSGQNQQT (SEQ ID NO: 201) demonstrated a 62.6 fold enrichment in the brain, a 15.6 fold enrichment in the spinal cord, and 0.0 fold enrichment in the DRG of NHPs relative to AAV9. Also, across the peptides comprised within the AAV capsid variants with the greatest fold-enrichment in the NHP brain relative wild-type AAV9, it was observed that each of these peptides comprised an SPH motif in the same position (e.g., immediately subsequent to position 455, relative to a reference sequence numbered according to the amino acid sequence of SEQ ID NO: 138), regardless of the insertion position within the variant capsid, as well as a positive amino acid (e.g., K or R) in one of the next three residues subsequent to the SPH motif.

Those variants with the greatest fold enrichment in the brains of NHPs also had the greatest fold enrichment in the brains of both mouse species. Also, when comparing the fold enrichment relative to wild-type for each variant between the two species of mice investigated (C57Bl/6 and BALB/c mice), they were highly correlated (R²=0.8591).

TABLE 9
NGS fold-enrichment of AAV capsid variants in NHPs and mice

| | | | | Fold | Fold |
|---|---|---|---|---|---|
| | | Fold | Fold | enrichment | enrichment |
| | | enrichment | enrichment | over | over |
| | | over AAV9 | over AAV9 in | AAV9 in the | AAV9 in the |
| Peptide | SEQ | in the brain | the spinal cord | brain of Mice | brain of Mice |
| Sequence | ID NO: | of NHPs | of NHPs | (C57BI/6) | (BALB/c) |
| GSGSPHSKAQNQQT | 200 | 73.615 | 29.402 | 25.293 | 41.304 |
| GHDSPHKSGQNQQT | 201 | 62.612 | 15.641 | 63.993 | 49.760 |
| GSGSPHARMQNQQT | 202 | 56.138 | 22.690 | 7.795 | 4.164 |
| GSGSPHVKSQNQQT | 203 | 37.551 | 13.649 | 8.069 | 15.861 |
| GQDSPHKSGQNQQT | 204 | 24.569 | 3.548 | 57.344 | 42.615 |
| GSGSPHASRQNQQT | 205 | 18.265 | 7.804 | 28.028 | 36.577 |
| GSGSPHASRQNKQT | 206 | 17.520 | 35.029 | 13.096 | 18.114 |
| GSGSPHVKIQNQQT | 207 | 16.854 | 9.068 | 2.173 | 2.227 |
| GSGSPHSKAKNQQT | 208 | 14.458 | 0.049 | 21.494 | 23.556 |
| GSGSPHKKNQNQQT | 209 | 12.991 | 0.379 | 25.958 | 7.415 |
| GSGSPHVRMQNQQT | 210 | 11.574 | 6.764 | 9.121 | 10.076 |
| GSGSPHASRQKQQT | 211 | 11.417 | 0.005 | 7.413 | 12.400 |
| GHSSPHRSGQNQQT | 212 | 10.357 | 1.887 | 23.197 | 25.442 |
| GMRTYHLSGQNQQT | 213 | 9.241 | 1.939 | 2.033 | 1.586 |
| GSGSPHTRGQNQQT | 214 | 7.092 | 3.815 | 10.801 | 6.240 |
| GSGIIPVSSQNQQT | 215 | 6.352 | 0.000 | 0.642 | 0.253 |
| GSEYGHKSGQNQQT | 216 | 6.308 | 2.750 | 5.198 | 5.332 |
| GRGQNVSSVHRQQT | 217 | 5.404 | 0.000 | 1.206 | 0.691 |
| GSSHRFYGGQNQQT | 218 | 4.732 | 0.000 | 0.787 | 0.110 |
| GYFVAAWSGQNQQT | 219 | 4.488 | 0.000 | 0.071 | 0.175 |
| GSVLHSHAAQNQQT | 220 | 4.150 | 6.448 | 0.675 | 0.423 |
| GSGDLVVSTQNQQT | 221 | 3.874 | 1.177 | 0.411 | 0.273 |
| GSYGMAASGQNQQT | 222 | 3.817 | 10.052 | 1.274 | 0.829 |
| GLNHFGASGQNQQT | 223 | 3.802 | 3.188 | 0.774 | 0.579 |
| GSTGSHSAGQNQHT | 224 | 3.717 | 0.285 | 1.190 | 0.850 |
| GLAGHTVSGQNQQT | 225 | 3.632 | 0.229 | 0.972 | 0.202 |
| GIILGASSGQNQQT | 226 | 3.630 | 4.868 | 1.378 | 0.865 |
| GSGVSTYNIQNQQT | 227 | 3.609 | 2.912 | 0.769 | 0.520 |
| GSLVSVQTGQNQQT | 228 | 3.534 | 6.043 | 0.903 | 0.469 |
| GQSSPHRSGQNQQT | 229 | 3.496 | 2.142 | 12.352 | 19.366 |
| GREYGHKSGQNQQT | 230 | 3.453 | 0.000 | 1.422 | 0.959 |
| GHTLTLSSGQNQQT | 231 | 3.405 | 5.648 | 0.648 | 0.606 |
| GSITLIPSGQNQQT | 232 | 3.361 | 3.917 | 0.326 | 0.435 |
| GSNGFTALGQNQQT | 233 | 3.361 | 2.663 | 0.830 | 0.332 |
| GSGHSSHSVQNQQT | 234 | 3.339 | 3.318 | 0.942 | 0.424 |
| GSGIPQRSGKNQQT | 235 | 3.331 | 0.000 | 1.418 | 1.685 |
| GSGDTLHMLQNQQT | 236 | 3.317 | 1.174 | 0.393 | 0.482 |
| GERHTVLSGQNQQT | 237 | 3.289 | 3.008 | 1.027 | 0.607 |
| GSGMPQSHIQNQQT | 238 | 3.289 | 11.609 | 0.514 | 0.334 |
| GSGQLSGIGGNQQT | 239 | 3.266 | 0.287 | 0.993 | 0.626 |
| GSGQNRKPASFAQT | 240 | 3.204 | 0.000 | 0.892 | 1.061 |
| GSGSVSQLGQNQQT | 241 | 3.184 | 2.307 | 0.596 | 0.375 |
| GSDFLGTHGQNQQT | 242 | 3.171 | 0.348 | 1.038 | 0.750 |
| GQIVQNPSGQNQQT | 243 | 3.133 | 0.406 | 1.446 | 0.635 |
| GSGTQIPSQQNQQT | 244 | 3.112 | 1.224 | 0.470 | 0.151 |
| GSGQNQQSAREGLT | 245 | 3.111 | 5.632 | 1.221 | 1.104 |
| GSGLGMSTGQNQQT | 246 | 3.110 | 5.499 | 0.458 | 0.660 |
| GSGLPVLSGQNQQT | 247 | 3.100 | 4.149 | 0.631 | 0.210 |
| GSGHSIRTDQNQQT | 248 | 3.074 | 15.600 | 0.229 | 0.148 |
| GSGQSVQTVVNQQT | 249 | 3.057 | 5.441 | 0.582 | 0.240 |
| GSGQNRAQSRFQQT | 250 | 3.043 | 0.000 | 0.619 | 1.788 |
| GGGDLGRSSQNQQT | 251 | 3.036 | 4.830 | 0.916 | 0.539 |
| GGGTKMDSGQNQQT | 252 | 3.034 | 0.000 | 0.733 | 0.297 |
| GSGSPHPSRQNQQT | 253 | 3.017 | 1.993 | 1.869 | 0.975 |
| GSGQFTNAGMNQQT | 254 | 2.969 | 0.936 | 0.565 | 0.418 |
| GGRNGHTVGQNQQT | 255 | 2.965 | 3.732 | 1.105 | 1.003 |
| GSGFGPQTGQNQQT | 256 | 2.964 | 2.861 | 1.280 | 0.849 |
| GRTDSHTSGQNQQT | 257 | 2.913 | 1.510 | 1.299 | 0.704 |
| GYEVLGSSGQNQQT | 258 | 2.891 | 0.000 | 2.459 | 0.319 |
| GSVHLSVTGQNQQT | 259 | 2.882 | 1.157 | 0.741 | 0.282 |
| GFMSYKGSGQNQQT | 260 | 2.865 | 0.209 | 1.808 | 0.569 |
| GNIAGSVSGQNQQT | 261 | 2.849 | 1.187 | 0.446 | 0.257 |
| GSGSHRDVSQNQQT | 262 | 2.843 | 4.022 | 0.626 | 0.550 |
| GGLGSMSSGQNQQT | 263 | 2.812 | 1.405 | 1.802 | 0.822 |
| GSGHLPQSAQNQQT | 264 | 2.803 | 7.828 | 0.826 | 0.496 |
| GGVLVGGSGQNQQT | 265 | 2.778 | 0.178 | 1.527 | 0.688 |
| GTHPYTSSGQNQQT | 266 | 2.775 | 1.684 | 0.758 | 0.471 |
| GSGQNQQLKENRST | 267 | 2.765 | 0.062 | 1.149 | 1.118 |
| GSGQNQQTSPHNHT | 268 | 2.761 | 3.132 | 1.524 | 0.845 |
| GSGTLYPQSQNQQT | 269 | 2.761 | 5.558 | 0.324 | 0.160 |
| GSGQNQQSNWITKT | 270 | 2.711 | 0.000 | 0.540 | 0.634 |
| GSGYTSLFLQNQQT | 271 | 2.710 | 0.010 | 0.490 | 1.044 |
| GSGVMTHVLQNQQT | 272 | 2.692 | 0.347 | 0.370 | 0.533 |
| GSVSDVRAGQNQQT | 273 | 2.661 | 1.647 | 0.267 | 0.747 |
| GSGQSHMATLNQQT | 274 | 2.657 | 0.724 | 1.173 | 0.504 |
| GSGLSVHLAQNQQT | 275 | 2.657 | 1.234 | 0.806 | 0.508 |
| GSGLSHATQQNQQT | 276 | 2.640 | 7.819 | 1.111 | 0.638 |
| GSGLSVQSGQNQQT | 277 | 2.637 | 2.929 | 1.695 | 1.005 |
| GSGHMTYREKNQQT | 278 | 2.633 | 5.267 | 1.257 | 0.540 |
| GSKGVPTPGQNQQT | 279 | 2.625 | 1.292 | 1.452 | 0.459 |
| GSGLLPLSSQNQQT | 280 | 2.612 | 1.130 | 0.501 | 0.293 |
| GNGLYAVSGQNQQT | 281 | 2.611 | 9.148 | 0.322 | 0.213 |
| GFNGSPSSGQNQQT | 282 | 2.609 | 12.197 | 2.338 | 0.924 |
| GSGQIRHSDQNQQT | 283 | 2.600 | 12.884 | 1.170 | 0.320 |
| GGQVAPSSGQNQQT | 284 | 2.581 | 2.427 | 1.433 | 0.709 |
| GSFSMHTHGQNQQT | 285 | 2.535 | 0.118 | 1.027 | 0.693 |
| GSGQNQQVIQGSNT | 286 | 2.521 | 8.778 | 0.935 | 0.810 |
| GRVLHSHAGQNQQT | 287 | 2.513 | 0.826 | 1.294 | 0.908 |
| GSGQNQQTSLQDQT | 288 | 2.505 | 0.500 | 0.315 | 0.968 |
| GSGLGRAPVQNQQT | 289 | 2.503 | 2.214 | 0.841 | 0.383 |
| GNGFSSASGQNQQT | 290 | 2.493 | 0.772 | 0.240 | 0.182 |
| GSGQMASRESNQQT | 291 | 2.492 | 0.300 | 0.341 | 0.288 |
| GPGLPNHSGQNQQT | 292 | 2.486 | 1.992 | 1.197 | 0.659 |
| GNIQWQGSGQNQQT | 293 | 2.468 | 6.266 | 1.182 | 0.837 |
| GMSAHMSSGQNQQT | 294 | 2.456 | 5.255 | 1.310 | 0.947 |
| GHSFVNRSGQNQQT | 295 | 2.447 | 11.148 | 1.305 | 0.756 |
| GRAVMDHSGQNQQT | 296 | 2.408 | 3.209 | 0.728 | 0.283 |
| GALTVMQSGQNQQT | 297 | 2.381 | 0.430 | 0.246 | 0.199 |
| GSGQRSPVLPNQQT | 298 | 2.369 | 6.230 | 0.434 | 0.526 |
| GSGQNGHLSLKQQT | 29 | 2.362 | 1.896 | 0.718 | 0.270 |
| GSLPRGTSDQNQQT | 300 | 2.362 | 0.000 | 0.453 | 0.495 |
| GVAGSLVSGQNQQT | 301 | 2.358 | 7.670 | 1.321 | 1.160 |
| GRGGIPQSGQNQQT | 302 | 2.352 | 8.683 | 1.639 | 1.181 |
| GSGQYASSIPNQQT | 303 | 2.346 | 3.321 | 1.022 | 0.489 |
| GTDFGRQSSQNQQT | 304 | 2.346 | 3.196 | 1.021 | 0.797 |
| GIFMQTPSGQNQQT | 305 | 2.344 | 6.198 | 0.938 | 0.252 |
| GSGQNQQTRLVDLT | 306 | 2.342 | 9.348 | 1.268 | 0.490 |
| GTREMPLSGQNQQT | 307 | 2.339 | 2.830 | 1.436 | 0.538 |
| GSRLVHVHGQNQQT | 308 | 2.334 | 1.174 | 1.277 | 0.934 |
| GSGRLVPNGPNQQT | 309 | 2.314 | 3.925 | 0.639 | 0.411 |
| GSGYLRESPQNQQT | 310 | 2.311 | 0.878 | 0.331 | 0.677 |
| GARIQNASGQKQQT | 311 | 2.300 | 2.103 | 1.220 | 1.039 |
| GLSNPMPSGQNQQT | 312 | 2.280 | 6.033 | 1.190 | 0.829 |
| GSTVQDTRGQNQQT | 313 | 2.270 | 4.979 | 0.576 | 0.473 |
| GPFGMPSSGQNQQT | 314 | 2.260 | 2.700 | 0.727 | 0.560 |
| GSGQNHGVLSNQQT | 315 | 2.254 | 1.603 | 1.113 | 0.701 |
| GSGYSMSQAQNQQT | 316 | 2.250 | 4.479 | 0.519 | 0.329 |
| GSGMLTHTLQNQQT | 317 | 2.246 | 2.272 | 0.496 | 0.199 |
| GRGSPHASRQNQQT | 318 | 2.241 | 0.000 | 5.050 | 5.856 |
| GLSWPSTSGQNQQT | 319 | 2.238 | 0.000 | 0.910 | 0.610 |
| GNSMERTSGQNQQT | 320 | 2.221 | 4.177 | 1.047 | 0.935 |
| GSGMSPSTLQNQQT | 321 | 2.216 | 3.053 | 0.318 | 0.153 |
| GSGHGQVLSQNQQT | 322 | 2.213 | 12.133 | 1.880 | 0.661 |
| GRGQIYSTGGNQQT | 323 | 2.210 | 11.629 | 1.329 | 0.743 |
| GVVAAHNSGQNQQT | 324 | 2.202 | 1.301 | 1.196 | 1.336 |
| GDSSLRHSGQNQQT | 325 | 2.194 | 0.000 | 0.662 | 0.412 |
| GSLVSQGAGQNQQT | 326 | 2.188 | 4.414 | 1.436 | 1.246 |
| GSLLQAHSGQNQQT | 327 | 2.182 | 1.008 | 0.575 | 0.748 |
| GSGHIYVGIQNQQT | 328 | 2.178 | 6.428 | 0.989 | 0.337 |
| GHHTTVQSGQNQQT | 329 | 2.177 | 6.245 | 0.851 | 0.755 |
| GSRQSKRNELNQQT | 330 | 2.177 | 0.000 | 1.325 | 0.232 |
| GSGQNQQHVSSPRT | 331 | 2.176 | 1.279 | 1.847 | 0.938 |
| GSSKELLWGQNQQT | 332 | 2.163 | 0.000 | 0.506 | 0.883 |
| GSLSTPSSGQNQQT | 333 | 2.159 | 1.279 | 1.094 | 0.669 |
| GSIGYAGQGQNQQT | 334 | 2.157 | 4.951 | 1.604 | 0.712 |
| GSGQNQRVSNSQQT | 335 | 2.146 | 0.492 | 1.086 | 0.985 |
| GSGYASHVQQNQQT | 336 | 2.146 | 3.038 | 1.157 | 0.758 |
| GSGEYSRSGQNQQT | 337 | 2.145 | 0.745 | 0.617 | 0.205 |
| GSVSTHSSGQNQQT | 338 | 2.145 | 3.446 | 1.198 | 0.918 |
| GSGQNQHSLGNYQT | 339 | 2.143 | 1.896 | 1.077 | 0.606 |
| GSGGLDTRGQNQQT | 340 | 2.139 | 6.216 | 0.236 | 0.197 |
| GNILHATSGQNQQT | 341 | 2.136 | 0.125 | 1.159 | 0.424 |
| GSGQSYTMTQNQQT | 342 | 2.136 | 6.755 | 0.297 | 0.231 |
| GSGQNQHSAPNSQT | 343 | 2.134 | 4.143 | 1.187 | 0.731 |
| GSGQNQQTMDHNRT | 344 | 2.130 | 4.944 | 0.642 | 0.440 |
| GSNGGVGTGQNQQT | 345 | 2.130 | 0.788 | 1.191 | 1.087 |
| GAGSIIPSGQNQQT | 346 | 2.129 | 7.164 | 0.595 | 0.249 |
| GSGQTHGGQHNQQT | 347 | 2.125 | 12.251 | 1.448 | 1.098 |
| GSNLSFQSGQNQQT | 348 | 2.122 | 5.853 | 1.087 | 0.719 |
| GATLQVHSGQNQQT | 349 | 2.122 | 2.219 | 0.623 | 0.545 |
| GSGFNQRSEQNQQT | 350 | 2.121 | 4.491 | 1.770 | 0.758 |
| GSGSLRDFDQNQQT | 351 | 2.120 | 6.846 | 0.586 | 0.272 |
| GSGDSITGKQNQQT | 352 | 2.112 | 1.295 | 0.793 | 0.306 |
| GSGQDRNIVQNQQT | 353 | 2.112 | 0.229 | 0.454 | 0.632 |
| GSGLSHSHQQNQQT | 354 | 2.109 | 5.852 | 1.256 | 0.592 |
| GSGQNQQTGMSSVK | 355 | 2.109 | 4.544 | 1.451 | 0.679 |
| GSVTHGISGQNQQT | 356 | 2.105 | 4.542 | 1.135 | 0.789 |
| GVVAHQPSGQNQQT | 357 | 2.103 | 0.152 | 0.910 | 2.267 |
| GSGPILGQLQNQQT | 358 | 2.097 | 2.058 | 0.470 | 0.123 |
| GSGHVPNSGLNQQT | 359 | 2.091 | 0.653 | 1.636 | 1.154 |
| GDAGVRSSGQNQQT | 360 | 2.068 | 3.918 | 1.033 | 1.193 |
| GSGSQLMSLQNQQT | 361 | 2.065 | 3.559 | 0.563 | 0.172 |
| GSGLDYSQRQNQQT | 362 | 2.056 | 0.837 | 0.484 | 0.217 |
| GSGQSSGRLINKQT | 363 | 2.055 | 28.135 | 0.543 | 0.277 |
| GSSVSPSSGQNQQT | 364 | 2.054 | 0.579 | 1.064 | 0.787 |
| GSGQVVGLSGNQQT | 365 | 2.052 | 7.212 | 0.785 | 0.881 |
| GSNMGVPLGQNQQT | 366 | 2.049 | 2.448 | 0.334 | 0.420 |
| GSFYPSSTGQNQQT | 367 | 2.047 | 2.374 | 0.420 | 0.277 |
| GSGQNQQTRLTDLT | 368 | 2.046 | 8.470 | 0.910 | 0.776 |
| GPTNGRSSGQNQQT | 369 | 2.034 | 8.903 | 0.936 | 1.308 |
| GSGLLHGKLQNQQT | 370 | 2.032 | 2.521 | 1.068 | 0.917 |
| GANMGHVSGQNQQT | 371 | 2.020 | 0.810 | 1.302 | 1.138 |
| GSGQNQQSGRGDLT | 372 | 2.019 | 6.919 | 0.524 | 1.152 |
| GSHGHYASGQKQQT | 373 | 2.016 | 0.000 | 0.895 | 0.685 |
| GSGDLRISPQNQQT | 374 | 2.012 | 16.207 | 0.620 | 0.237 |
| GSGMPVILGQNQQT | 375 | 2.005 | 0.150 | 0.840 | 0.287 |
| GRGVITSSGQNHQT | 376 | 2.004 | 0.864 | 1.656 | 0.669 |
| GSGHSVSGPQNQQT | 377 | 1.993 | 6.259 | 1.370 | 0.619 |
| GSRNGHTVGRNQQT | 378 | 1.993 | 0.000 | 1.162 | 0.367 |
| GAGVHMVSGQNQQT | 379 | 1.987 | 6.488 | 1.055 | 0.791 |
| GSGQNHRPSVLQQT | 380 | 1.983 | 5.582 | 0.433 | 0.582 |
| GSGSPRDSIQNQQT | 381 | 1.981 | 4.914 | 0.171 | 0.446 |
| GSGQGIHSSVNQQT | 382 | 1.981 | 4.873 | 0.632 | 0.634 |
| GSGQQLSITPNQQT | 383 | 1.979 | 10.280 | 0.845 | 0.201 |
| GGYHSQTSGQNQQT | 384 | 1.978 | 2.642 | 1.740 | 1.525 |
| GSLHHDNHGQNQQT | 385 | 1.976 | 0.980 | 0.968 | 0.463 |
| GIMARDSSGQNQQT | 386 | 1.972 | 3.486 | 1.320 | 0.904 |
| GVVHITNSGQNQQT | 387 | 1.969 | 0.504 | 0.794 | 0.846 |
| GSGQNQHSAPFNQT | 388 | 1.969 | 0.499 | 0.759 | 0.870 |
| GSGQTSGLKQNQQT | 389 | 1.968 | 3.927 | 0.394 | 0.334 |
| GSGQNQQTSLSNTA | 390 | 1.959 | 1.186 | 1.567 | 1.182 |
| GSGQNQAVHNKSQT | 391 | 1.956 | 3.791 | 1.465 | 1.083 |
| GVHTHLPSGQNQQT | 392 | 1.952 | 1.364 | 1.414 | 0.796 |
| GHLTMHNSGQNHQT | 393 | 1.938 | 1.798 | 1.030 | 0.586 |
| GSGSSSRPYQNQQT | 394 | 1.934 | 3.823 | 0.962 | 0.496 |
| GILLATPSGQNQQT | 395 | 1.931 | 8.205 | 1.341 | 0.288 |
| GSGQNAGSFPNQQT | 396 | 1.928 | 12.575 | 1.091 | 0.286 |
| GSRDGHTVGQNQQT | 397 | 1.928 | 7.089 | 0.495 | 0.661 |
| GSLLISTSGQNQQT | 398 | 1.919 | 5.763 | 1.488 | 0.808 |
| GSGAMPSHGQNQQT | 399 | 1.915 | 0.000 | 1.142 | 0.912 |
| GALVSPISGQNQQT | 400 | 1.912 | 1.051 | 0.640 | 0.347 |
| GSLSSHGVGQNQQT | 401 | 1.911 | 7.498 | 1.218 | 0.804 |
| GSGQNQQASLAMRT | 402 | 1.910 | 3.577 | 2.066 | 1.638 |
| GPGLGSHSGQNQQT | 403 | 1.906 | 14.563 | 0.880 | 1.195 |
| GHDSQHKSGQNQQT | 404 | 1.904 | 6.988 | 1.154 | 0.869 |
| GSGLTLSATQNQQT | 405 | 1.901 | 0.193 | 0.708 | 0.340 |
| GSGQVVAHVGNQQT | 406 | 1.901 | 0.833 | 0.800 | 0.321 |
| GSGLRTMTTQNQQT | 407 | 1.900 | 8.939 | 0.838 | 0.594 |
| GSGQVGRLLQNQQT | 408 | 1.899 | 1.762 | 0.773 | 0.748 |
| GSGQLSHQSVNQQT | 409 | 1.898 | 4.032 | 0.720 | 0.695 |
| GSGDRYQTLQNQQT | 410 | 1.897 | 1.075 | 0.645 | 0.318 |
| GSGQNQQLKSSAQT | 411 | 1.891 | 1.197 | 0.908 | 0.716 |
| GSGQNQYSIPVAQT | 412 | 1.891 | 1.194 | 0.511 | 0.297 |
| GSGERLHLTQNQQT | 413 | 1.885 | 1.456 | 0.387 | 0.245 |
| GSGHNQQVRTAPNT | 414 | 1.885 | 1.022 | 1.006 | 0.580 |
| GGLSHVMSGQNQQT | 415 | 1.875 | 0.403 | 0.885 | 0.378 |
| GSGQSHRDVLNQQT | 416 | 1.872 | 15.082 | 0.138 | 0.280 |
| GSGQNLAGRMDQQT | 417 | 1.864 | 0.085 | 0.362 | 0.295 |
| GSGQNQQTNRGNPM | 418 | 1.860 | 3.402 | 1.349 | 1.098 |
| GSGQSYQRDHNQQT | 419 | 1.859 | 8.013 | 0.779 | 0.323 |
| GSLLSAGMGQNQHT | 420 | 1.856 | 6.168 | 0.589 | 0.342 |
| GSGQNQQTAIYRNI | 421 | 1.854 | 2.207 | 0.818 | 1.437 |
| GSGQNQQTSGTTNC | 422 | 1.854 | 8.161 | 1.040 | 0.806 |
| GMTSHSVSGQNQQT | 423 | 1.850 | 2.732 | 0.220 | 0.154 |
| GSSQSTGYQPNQQT | 424 | 1.847 | 3.388 | 0.522 | 0.577 |
| GSLKPTTLGQNQQT | 425 | 1.840 | 0.476 | 0.175 | 0.220 |
| GRMFSLGSGQNQQT | 426 | 1.836 | 8.429 | 1.630 | 1.174 |
| GSGQNQQTALGVKC | 427 | 1.835 | 1.343 | 1.378 | 1.014 |
| GAMVSHSSGQNQQT | 428 | 1.833 | 8.999 | 0.739 | 0.868 |
| GSGQNQQRNSDSVT | 429 | 1.829 | 0.000 | 1.238 | 0.842 |
| GSGQSMTLHLNQQT | 430 | 1.827 | 0.991 | 0.721 | 0.248 |
| GSGQVHQAEVNQQT | 431 | 1.825 | 0.152 | 0.436 | 0.287 |
| GSGQNQSQNHLQQT | 432 | 1.825 | 0.600 | 1.063 | 0.772 |
| GSLLTTASGQNQQT | 433 | 1.822 | 0.780 | 0.938 | 0.635 |
| GSGLIRTAAQNQQT | 434 | 1.822 | 8.339 | 0.808 | 0.998 |
| GSGQNQQTVSRQST | 435 | 1.820 | 0.472 | 1.330 | 0.796 |
| GSGQYANHGINQQT | 436 | 1.820 | 5.717 | 0.906 | 0.701 |
| GSRSTGPSGQNQQT | 437 | 1.819 | 2.479 | 0.440 | 0.466 |
| GRGVQQKLQQNQQT | 438 | 1.817 | 0.000 | 1.974 | 0.823 |
| GSGQNQQVHLSTGT | 439 | 1.811 | 0.266 | 1.011 | 0.455 |
| GSGQNQQLSAKSST | 440 | 1.809 | 1.567 | 1.224 | 1.115 |
| GSGYKAARPQNQQT | 441 | 1.803 | 0.000 | 1.418 | 0.337 |
| GSAGISPSGQNQQT | 442 | 1.797 | 1.812 | 0.784 | 0.622 |
| GSGQNRAHAFLQQT | 443 | 1.795 | 0.000 | 1.200 | 1.271 |
| GSGLSGITMQNQQT | 444 | 1.792 | 14.796 | 0.862 | 0.496 |
| GPGSAHSSGQNQQT | 445 | 1.785 | 4.392 | 1.099 | 0.872 |
| GSSHTQALGQNQQT | 446 | 1.784 | 0.143 | 0.882 | 0.874 |
| GSGVHGVSSQNQQT | 447 | 1.781 | 4.519 | 1.504 | 0.951 |
| GSSGRDMGGQNQQT | 448 | 1.778 | 2.177 | 1.052 | 0.595 |
| GERAFPTSGQNQQT | 449 | 1.775 | 6.515 | 0.972 | 0.362 |
| GGRIVSLSGQNQQT | 450 | 1.766 | 4.936 | 1.161 | 0.847 |
| GSGQNSYSHTSQQT | 451 | 1.765 | 2.262 | 1.130 | 0.658 |
| GLGYPGSSGQNQQT | 452 | 1.763 | 7.090 | 0.929 | 0.577 |
| GSGPQSHTGQNQQT | 453 | 1.757 | 9.490 | 0.958 | 0.447 |
| GSGQNQQLSRDAST | 454 | 1.754 | 3.716 | 1.877 | 0.611 |
| GSGQILHSVPNQQT | 455 | 1.752 | 1.316 | 0.398 | 0.240 |
| GSGEHTDSRQNQQT | 456 | 1.748 | 4.384 | 7.344 | 0.575 |
| GSGQSHSLATNQQT | 457 | 1.745 | 2.711 | 1.021 | 0.343 |
| GSGQNQQTLSKPWT | 458 | 1.743 | 0.253 | 0.845 | 0.733 |
| GSGHAAISQQNQQT | 459 | 1.742 | 2.373 | 1.211 | 0.520 |
| GSGQNQQQIGGNST | 460 | 1.741 | 6.169 | 0.877 | 0.576 |
| GGGPMAGSGQNQQT | 461 | 1.735 | 2.815 | 1.049 | 0.372 |
| GMRMEYQSGQNQQT | 462 | 1.729 | 3.695 | 0.644 | 0.632 |
| GSGQNQQGTLLHQT | 463 | 1.728 | 2.065 | 1.347 | 1.303 |
| GSGQNQRSSGGVQT | 464 | 1.723 | 2.056 | 1.805 | 1.165 |
| GSGQNQRGALATQT | 465 | 1.722 | 1.117 | 0.899 | 0.891 |
| GSGTVHAATQNQQT | 466 | 1.721 | 1.676 | 0.563 | 0.476 |
| GSRMTQQFGQNQQT | 467 | 1.720 | 25.798 | 1.233 | 0.976 |
| GSSSPGASGQNQQT | 468 | 1.717 | 1.244 | 1.378 | 0.660 |
| GHPSPHVSGQNQQT | 469 | 1.713 | 0.416 | 0.551 | 0.488 |
| GSGSHHASRQNQQT | 470 | 1.712 | 0.451 | 3.073 | 0.584 |
| GAVGHSYSGQNQQT | 471 | 1.706 | 0.808 | 0.306 | 0.536 |
| GSRSQYDIGQNQQT | 472 | 1.706 | 0.112 | 0.528 | 0.193 |
| GSGQGPQERGNQQT | 473 | 1.702 | 1.269 | 0.846 | 0.313 |
| GSIAHVGTGQNQQT | 474 | 1.696 | 1.264 | 0.837 | 1.045 |
| GSGQNQQKQNHGNT | 475 | 1.695 | 5.349 | 1.538 | 1.340 |
| GSGQNQQALGSQRT | 476 | 1.695 | 1.934 | 1.419 | 0.562 |
| GSGAITHMPQNQQT | 477 | 1.695 | 1.681 | 0.647 | 0.411 |
| GSGQRNPLLLNQQT | 478 | 1.693 | 0.144 | 0.662 | 0.740 |
| GSSGIPVSHQNQQT | 479 | 1.690 | 3.384 | 0.820 | 0.333 |
| GVHSLTPSGQNQQT | 480 | 1.687 | 4.104 | 0.475 | 0.215 |
| GVIVLHGSGQNQQT | 481 | 1.682 | 14.166 | 1.074 | 1.098 |
| GGTRVVDSGQNQQT | 482 | 1.676 | 9.735 | 0.676 | 0.370 |
| GSGGVTYQSQNQQT | 483 | 1.673 | 7.283 | 0.649 | 0.181 |
| GSGQNQAGHGPGQT | 484 | 1.670 | 2.861 | 1.040 | 0.887 |
| GSGQLVTSGPNQQT | 485 | 1.669 | 5.271 | 0.964 | 0.433 |
| GSGIAAQRTQNQQT | 486 | 1.665 | 2.691 | 1.062 | 0.754 |
| GSTPAGVGGQNQQT | 487 | 1.663 | 2.733 | 0.593 | 0.477 |
| GSGQNQQTSTGVHS | 488 | 1.660 | 8.271 | 1.039 | 1.075 |
| GSGQIRQLVDNQQT | 489 | 1.657 | 5.529 | 0.314 | 0.272 |
| GSLIGMQSGQNQQT | 490 | 1.656 | 6.783 | 0.797 | 0.392 |
| GSGQIKGKMDNQQT | 491 | 1.654 | 2.601 | 1.065 | 1.012 |
| GSGSDMSSWQNQQT | 492 | 1.651 | 0.175 | 0.281 | 0.303 |
| GRGQNQQHTGLATT | 493 | 1.650 | 6.174 | 1.134 | 0.691 |
| GSGQNQQTLYSSNT | 494 | 1.642 | 1.044 | 0.664 | 0.368 |
| GSGQTQVLKSNQQT | 495 | 1.640 | 3.031 | 1.599 | 0.975 |
| GSRTLSNVGQNQQT | 496 | 1.640 | 3.219 | 0.617 | 0.542 |
| GSGVQHSLPQNQQT | 497 | 1.639 | 0.764 | 0.440 | 0.387 |
| GNYLHQASGQNQQT | 498 | 1.635 | 1.454 | 0.816 | 0.181 |
| GSGGTSVHQQNQQT | 499 | 1.629 | 0.000 | 0.585 | 0.195 |
| GMDHSRPSGQNQQT | 500 | 1.627 | 3.976 | 0.918 | 0.648 |
| GSGQNQQSMGTFTT | 501 | 1.625 | 0.000 | 1.792 | 0.399 |
| GSGQNQQTPLRPPT | 502 | 1.624 | 0.352 | 0.874 | 0.472 |
| GSGQNQHHSVSQQT | 503 | 1.623 | 3.700 | 0.605 | 0.334 |
| GSGQLRSLSTNQQT | 504 | 1.622 | 6.855 | 1.310 | 0.382 |
| GSGSPRQLSQNQQT | 505 | 1.621 | 0.873 | 0.520 | 0.273 |
| GSGQNQQTTASSHT | 506 | 1.618 | 7.404 | 0.745 | 0.678 |
| GRGQVVSTHQNQQT | 507 | 1.607 | 3.318 | 0.931 | 0.561 |
| GSAQVSMVGQNQQT | 508 | 1.601 | 1.332 | 0.500 | 0.285 |
| GSSTLVTIGKNQQT | 509 | 1.592 | 4.316 | 0.917 | 0.819 |
| GFAHQASSGQNQQT | 510 | 1.587 | 1.852 | 1.638 | 1.080 |
| GSGQPVLSISNQQT | 511 | 1.586 | 2.695 | 0.390 | 0.282 |
| GSGQSHRSELNQQT | 512 | 1.585 | 11.974 | 0.668 | 0.256 |
| GSSVGSPIGQNQQT | 513 | 1.584 | 3.574 | 1.059 | 0.706 |
| GSGMPIRNVQNQQT | 514 | 1.584 | 0.138 | 0.684 | 0.631 |
| GSSTRVDSGQNQQT | 515 | 1.584 | 2.774 | 0.704 | 0.660 |
| GSGQNQQTAMRSTT | 516 | 1.581 | 2.588 | 0.656 | 0.665 |
| GSGQNQQHSSSHLT | 517 | 1.581 | 2.782 | 1.091 | 0.859 |
| GSRNGHAVGQNQQT | 518 | 1.574 | 2.688 | 0.434 | 0.939 |
| GLGAYQSSGQNQQT | 519 | 1.574 | 0.696 | 1.407 | 0.688 |
| GPGLSGHSGQNQQT | 520 | 1.571 | 1.603 | 1.154 | 1.297 |
| GSTGIVSSGQNQQT | 521 | 1.570 | 0.927 | 2.141 | 1.046 |
| GSRTTQVIGQNQQT | 522 | 1.570 | 1.838 | 0.773 | 0.564 |
| GSGLLHRAQQNQQT | 523 | 1.569 | 0.724 | 1.583 | 0.646 |
| GSGQNAQQAAAQQT | 524 | 1.568 | 4.239 | 0.937 | 0.604 |
| GSGQNQQSALRTQT | 525 | 1.568 | 1.913 | 1.581 | 1.421 |
| GSGFLSDTRQNQQT | 526 | 1.566 | 45.953 | 0.473 | 0.575 |
| GSGLLYHDQQNQQT | 527 | 1.565 | 2.760 | 0.405 | 0.107 |
| GSGQNQHYSLHKQT | 528 | 1.563 | 3.399 | 1.485 | 1.273 |
| GSGHSPLPQQNQQT | 529 | 1.562 | 0.556 | 0.387 | 0.247 |
| GNGHSMRPNQNQQT | 530 | 1.560 | 2.341 | 0.693 | 0.376 |
| GSGLKWSTLQNQQT | 531 | 1.556 | 0.000 | 1.134 | 2.442 |
| GSGQMGRQAVNQQT | 532 | 1.554 | 1.529 | 0.535 | 0.411 |
| GSGQNQQTSGVLTL | 533 | 1.553 | 0.000 | 1.104 | 0.782 |
| GSGQNQQALHNPHT | 534 | 1.553 | 0.664 | 0.638 | 0.213 |
| GSGQNQQVIPNSKT | 535 | 1.548 | 1.036 | 0.844 | 0.376 |
| GSPLQDRVGQNQQT | 536 | 1.548 | 0.753 | 0.469 | 0.391 |
| GSGQNQYSSTNPQT | 537 | 1.542 | 2.251 | 0.544 | 0.535 |
| GAMTVTISGQNQQT | 538 | 1.542 | 6.249 | 0.443 | 0.257 |
| GSGQNQQLQTLIRT | 539 | 1.538 | 1.425 | 0.813 | 0.514 |
| GSGLRQTSQQNQQT | 540 | 1.537 | 2.067 | 0.978 | 0.705 |
| GSGQNQQTGLRQQT | 541 | 1.533 | 2.120 | 1.217 | 1.103 |
| GSGQTRQMKDNQQT | 542 | 1.530 | 11.079 | 0.841 | 0.214 |
| GSGQNHGLQSGQQT | 543 | 1.530 | 4.960 | 0.938 | 0.779 |
| GSGQSHRQPENQQT | 544 | 1.529 | 2.153 | 0.209 | 0.159 |
| GSGQDRHIVQNQQT | 545 | 1.527 | 11.068 | 0.285 | 0.162 |
| GSGQNQQLPHSNLT | 546 | 1.521 | 1.838 | 0.442 | 0.283 |
| GSGQLSVPYDNQQT | 547 | 1.521 | 0.000 | 0.622 | 0.111 |
| GSGRNPQTQPLQQT | 548 | 1.519 | 0.040 | 0.733 | 0.573 |
| GSGQPYSTGLNQQT | 549 | 1.519 | 1.403 | 0.612 | 0.376 |
| GSGQNQQTHGGLRD | 550 | 1.519 | 6.487 | 1.913 | 1.298 |
| GAYGMVSSGQNQQT | 551 | 1.518 | 3.469 | 0.732 | 0.773 |
| GSGIQSSYSQNQQT | 552 | 1.517 | 15.978 | 1.032 | 0.684 |
| GPRLSDQSGQNQQT | 553 | 1.511 | 0.364 | 0.640 | 0.579 |
| GSGQNQQTHPSPCT | 554 | 1.510 | 1.003 | 1.120 | 0.546 |
| GSGQSFQMHTNQQT | 555 | 1.504 | 9.770 | 0.503 | 0.325 |
| GSGQNQQTGNPKHT | 556 | 1.504 | 5.973 | 1.391 | 1.139 |
| GFSSAVHSGQNQQT | 557 | 1.502 | 1.234 | 0.218 | 0.210 |
| GSGQNQQTSMSNAT | 558 | 1.501 | 6.766 | 1.605 | 0.745 |
| GSGQDMKQHHNQQT | 559 | 1.501 | 1.638 | 0.358 | 0.239 |
| GIRLSTPSGQNQQT | 560 | 1.498 | 4.334 | 0.804 | 0.522 |
| GSGQNQQTSVYMNT | 561 | 1.498 | 0.613 | 0.640 | 0.983 |
| GSGQNQYSQSSMQT | 562 | 1.494 | 4.278 | 0.375 | 0.309 |
| GSGQNQQSMADHTT | 563 | 1.494 | 1.728 | 0.428 | 0.215 |
| GWERSFVSGQNQQT | 564 | 1.492 | 0.943 | 0.490 | 0.538 |
| GLLAGKSSGQNQQT | 565 | 1.491 | 2.981 | 0.999 | 0.946 |
| GKSFVPQSGQNQQT | 566 | 1.489 | 2.502 | 1.798 | 0.430 |
| GSGQMQSAGSNQQT | 567 | 1.482 | 0.116 | 1.034 | 1.128 |
| GSDQNQRLTSSMQT | 568 | 1.479 | 0.164 | 0.875 | 0.670 |
| GESRAVLSGQNQQT | 569 | 1.476 | 0.938 | 0.789 | 0.368 |
| GSVFGVPSGQNQQT | 570 | 1.474 | 1.248 | 0.685 | 0.213 |
| GSGLPDRNLQNQQT | 571 | 1.471 | 7.306 | 1.136 | 0.611 |
| GSGTHNSAIQNQQT | 572 | 1.469 | 0.570 | 0.762 | 0.574 |
| GSGMIIASMQNQQT | 573 | 1.469 | 6.722 | 1.135 | 0.415 |
| GGITWTDSGQNQQT | 574 | 1.462 | 4.535 | 1.472 | 0.468 |
| GSGQNQQASGRQQT | 575 | 1.458 | 3.179 | 0.943 | 0.991 |
| GSGQNQQPHLKSLT | 576 | 1.457 | 5.016 | 1.096 | 0.740 |
| GPPQHMTSGQNQQT | 577 | 1.457 | 1.547 | 0.509 | 0.677 |
| GSGQNQQASLPSRT | 578 | 1.456 | 0.389 | 0.930 | 0.673 |
| GSGQIVSTQTNQQT | 579 | 1.456 | 1.103 | 0.453 | 0.512 |
| GSGKGHSAGQNQQT | 580 | 1.453 | 0.936 | 1.035 | 1.173 |
| GSGQNTRLQLGQQT | 581 | 1.452 | 1.747 | 0.181 | 0.234 |
| GSVGSRPVGQNQQT | 582 | 1.442 | 11.363 | 1.182 | 0.716 |
| GSSHTLALGQNQQT | 583 | 1.441 | 7.071 | 0.851 | 0.406 |
| GMYEYSQSGQNQQT | 584 | 1.438 | 0.000 | 1.410 | 0.448 |
| GNGQNQQHSILHGT | 585 | 1.435 | 0.000 | 0.777 | 0.415 |
| GSGYNQPHLQNQQT | 586 | 1.435 | 4.512 | 0.711 | 0.395 |
| GPLVNASSGQNQQT | 587 | 1.434 | 5.239 | 0.831 | 0.343 |
| GSGQNQQVLTTART | 588 | 1.434 | 4.142 | 1.071 | 0.948 |
| GSGQNQHSVHNDQT | 589 | 1.428 | 0.000 | 0.521 | 0.515 |
| GAGLIMHSGQNQQT | 590 | 1.425 | 1.408 | 0.565 | 0.511 |
| GMGRHSASGQNQQT | 591 | 1.417 | 6.500 | 0.470 | 0.389 |
| GSHSQSGHGQNQQT | 592 | 1.413 | 1.240 | 0.696 | 0.318 |
| GSSTTIVSGQNHQT | 593 | 1.411 | 0.000 | 0.993 | 0.672 |
| GRHLVTASGQNQQT | 594 | 1.411 | 2.885 | 0.648 | 0.404 |
| GSGQNQQHANLNQT | 595 | 1.410 | 0.094 | 0.416 | 0.544 |
| GSGSTHSKAQNQQT | 596 | 1.410 | 0.515 | 0.921 | 0.801 |
| GSGQNKQMLSGNTT | 597 | 1.410 | 2.219 | 1.074 | 0.404 |
| GSGQVHNPTQNQQT | 598 | 1.410 | 2.488 | 1.021 | 0.542 |
| GSGQNQQIPHVHQT | 599 | 1.409 | 0.768 | 0.576 | 0.218 |
| GSLHAGLSGQNQQT | 600 | 1.408 | 1.739 | 1.286 | 0.936 |
| GPAQHGTSGQNQQT | 601 | 1.407 | 0.866 | 1.030 | 0.615 |
| GEKAVTSSGQNQQT | 602 | 1.402 | 0.998 | 0.558 | 0.327 |
| GSGQNQQTMANGQR | 603 | 1.394 | 0.216 | 1.169 | 1.230 |
| GSGSPHSKDQNQQT | 604 | 1.394 | 0.000 | 2.041 | 4.680 |
| GSFSMGYGGQNQQT | 605 | 1.393 | 18.476 | 1.908 | 1.030 |
| GSGTHLVSLQNQQT | 606 | 1.392 | 0.000 | 0.715 | 1.167 |
| GSGQMQPHVQNQQT | 607 | 1.389 | 9.381 | 0.387 | 0.153 |
| GSGQNQQVAGLNNT | 608 | 1.386 | 3.218 | 0.449 | 0.492 |
| GSSQNQQHDMRLRT | 609 | 1.386 | 2.645 | 0.669 | 0.552 |
| GPASLPISGQNQQT | 610 | 1.386 | 9.008 | 0.312 | 0.155 |
| GSGQNQQPPLATRT | 611 | 1.386 | 2.295 | 0.593 | 0.287 |
| GSSRVPVSGQNQQT | 612 | 1.385 | 13.191 | 0.870 | 0.485 |
| GSGQNQQTNLGHTT | 613 | 1.383 | 1.523 | 1.343 | 1.281 |
| GSGQNQQLVSRVQT | 614 | 1.381 | 1.195 | 0.656 | 0.466 |
| GPNSYPVSGQKQQT | 615 | 1.381 | 4.040 | 0.736 | 0.834 |
| GHAHYQASGQNQQT | 616 | 1.377 | 7.299 | 0.803 | 0.745 |
| GSGQALLSTGNQQT | 617 | 1.377 | 0.847 | 0.536 | 0.370 |
| GSGQLPRQMTNQQT | 618 | 1.376 | 3.550 | 0.400 | 0.562 |
| GSGFPKSTEQNQQT | 619 | 1.376 | 2.058 | 0.610 | 0.194 |
| GSRETSLSGQNQQT | 620 | 1.373 | 5.193 | 1.364 | 0.203 |
| GSGQNQQGTGVSHT | 621 | 1.371 | 4.295 | 1.417 | 0.749 |
| GSRTVPVYGQNQQT | 622 | 1.371 | 0.363 | 1.226 | 0.969 |
| GSNAQSAHGQNQQT | 623 | 1.371 | 0.888 | 0.976 | 0.245 |
| GAFHLAASGQNQQT | 624 | 1.369 | 18.165 | 0.994 | 0.775 |
| GSGQYRSSSDNQQT | 625 | 1.369 | 6.209 | 0.681 | 0.409 |
| GSGQVYISTPNQQT | 626 | 1.367 | 0.000 | 0.859 | 0.282 |
| GSGVSTQLLQNQQT | 627 | 1.367 | 2.467 | 0.928 | 0.509 |
| GSGQLGLSVTNQQT | 628 | 1.364 | 6.906 | 1.395 | 0.376 |
| GSGSNMRLSQNQQT | 629 | 1.363 | 0.588 | 0.962 | 0.730 |
| GSGQNLHSGLPQQT | 630 | 1.363 | 1.594 | 1.054 | 0.592 |
| GSSHTLALGQNKQT | 631 | 1.362 | 2.160 | 0.838 | 0.643 |
| GSGQNQHSLPAHRT | 632 | 1.361 | 0.700 | 0.911 | 0.742 |
| GSGQNQGTVYPNQT | 633 | 1.358 | 7.648 | 0.835 | 0.815 |
| GSGQNQQPSLRQST | 634 | 1.356 | 2.905 | 1.315 | 0.554 |
| GSGQNARLKDNQQT | 635 | 1.354 | 2.395 | 0.580 | 0.938 |
| GHAGSTGSGQNQQT | 636 | 1.352 | 2.829 | 1.332 | 1.233 |
| GSGQALSSSGNQQT | 637 | 1.351 | 6.860 | 0.894 | 0.931 |
| GSGASESHRQNQQT | 638 | 1.350 | 0.850 | 0.325 | 0.313 |
| GVGVITSSGQNQQT | 639 | 1.348 | 0.918 | 1.296 | 0.777 |
| GSLYGQSLGQNQQT | 640 | 1.348 | 11.248 | 0.894 | 0.843 |
| GSGQMSDVHGNQQT | 641 | 1.346 | 7.172 | 0.408 | 0.548 |
| GSGQNQQHSSKATT | 642 | 1.345 | 12.248 | 1.350 | 1.401 |
| GSGQNQQTSVSQQT | 643 | 1.342 | 1.614 | 1.030 | 0.913 |
| GSGQKMWKLDNQQT | 644 | 1.341 | 0.000 | 0.990 | 1.418 |
| GSGQNVSMQVNQQT | 645 | 1.341 | 0.000 | 0.357 | 0.251 |
| GSGQNQRATLSNQT | 646 | 1.339 | 1.084 | 0.947 | 0.723 |
| GSGQASSKSANQQT | 647 | 1.339 | 1.138 | 0.500 | 0.175 |
| GSGKNQTPIPKGQT | 648 | 1.339 | 5.077 | 1.306 | 1.154 |
| GSGQNQQTRQEGST | 649 | 1.339 | 0.000 | 0.645 | 0.718 |
| GASSLATSGQNQQT | 650 | 1.337 | 0.703 | 0.423 | 0.217 |
| GSGQRGSLTENQQT | 651 | 1.337 | 2.482 | 0.300 | 0.567 |
| GSEQTRQRGQNQQT | 652 | 1.333 | 2.172 | 0.574 | 0.815 |
| GSGQNQQTLTASKE | 653 | 1.333 | 1.152 | 0.981 | 1.172 |
| GSGTSGKTGKNQQT | 654 | 1.333 | 4.033 | 0.358 | 0.676 |
| GQLVTFTSGQNQQT | 655 | 1.331 | 11.282 | 0.819 | 0.294 |
| GSGQNQQSANKILT | 656 | 1.331 | 3.789 | 0.894 | 1.236 |
| GSGQNQQHHSSHTT | 657 | 1.328 | 2.158 | 0.957 | 0.452 |
| GSGQNQKGMQPNQT | 658 | 1.326 | 3.139 | 0.775 | 1.059 |
| GSGQLVSGLYNQQT | 659 | 1.325 | 0.000 | 0.842 | 0.733 |
| GSSVGVPSGQNQQT | 660 | 1.322 | 4.867 | 0.336 | 1.157 |
| GSGQNQQWDSRRQT | 661 | 1.321 | 0.531 | 1.059 | 0.825 |
| GSEQTRQSGQNQQT | 662 | 1.321 | 0.514 | 0.734 | 0.900 |
| GSGIGSHIPQNQQT | 663 | 1.319 | 0.173 | 0.822 | 0.597 |
| GSGQNQRLHGVDQT | 664 | 1.318 | 4.655 | 0.459 | 0.341 |
| GEVSRVLSGQNQQT | 665 | 1.318 | 0.437 | 1.150 | 0.440 |
| GSGQNQQKVSPLLT | 666 | 1.314 | 1.602 | 0.755 | 0.806 |
| GSGLALERSQNQQT | 667 | 1.311 | 0.486 | 0.618 | 0.096 |
| GPDRIGSSGQNQQT | 668 | 1.308 | 0.426 | 0.654 | 0.342 |
| GSGQNQDHQNKQQT | 669 | 1.308 | 1.470 | 0.510 | 0.761 |
| GSGQNQQTALYNNT | 670 | 1.307 | 0.862 | 0.660 | 0.726 |
| GSGAVHLTAQNQQT | 671 | 1.306 | 1.668 | 0.541 | 0.466 |
| GSLVSTQSGQNQQT | 672 | 1.305 | 1.293 | 1.282 | 0.650 |
| GSGVSARMVQNQQT | 673 | 1.299 | 0.624 | 0.870 | 0.697 |
| GSGQTRMPLANQQT | 674 | 1.296 | 0.790 | 0.447 | 0.273 |
| GSGISSRNMQNQQT | 675 | 1.291 | 6.328 | 1.671 | 0.560 |
| GSGEKVHSGQNQQT | 676 | 1.289 | 0.062 | 0.862 | 0.671 |
| GSGQNQQKLSSMST | 677 | 1.286 | 1.586 | 1.160 | 1.052 |
| GSGQNQQTGQHMRV | 678 | 1.286 | 4.161 | 1.839 | 1.635 |
| GSGMIHTTAQNQQT | 679 | 1.285 | 0.105 | 0.678 | 0.276 |
| GSGQNWPALKGQQT | 680 | 1.284 | 2.031 | 1.101 | 1.222 |
| GASHMSISGQNQQT | 681 | 1.284 | 0.462 | 0.404 | 0.374 |
| GSDQNQQLGYSKQT | 682 | 1.283 | 0.000 | 0.853 | 0.660 |
| GIPSIRESGQNQQT | 683 | 1.282 | 0.166 | 0.484 | 0.254 |
| GSGIPSVKFQNQQT | 684 | 1.281 | 0.061 | 0.364 | 0.561 |
| GSGQNQQTSVSQNV | 685 | 1.281 | 0.750 | 0.788 | 0.715 |
| GSGQNQQIGESRMT | 686 | 1.279 | 0.103 | 0.890 | 0.453 |
| GSGSSSMSFQNQQT | 687 | 1.279 | 0.540 | 0.466 | 0.095 |
| GSGQKQERAVSKQT | 688 | 1.277 | 0.000 | 1.174 | 0.732 |
| GCTTRLNSGQNQQT | 689 | 1.276 | 0.000 | 0.184 | 0.618 |
| GSGQNQQIISTKIT | 690 | 1.275 | 0.000 | 0.951 | 0.710 |
| GSGQNQQKSLNGNT | 691 | 1.275 | 8.573 | 0.586 | 0.851 |
| GSGIPAPRLQNQQT | 692 | 1.273 | 4.162 | 0.583 | 0.396 |
| GSGQIRESMGNQQT | 693 | 1.270 | 1.676 | 0.833 | 0.523 |
| GSGQNSGVHFNQQT | 694 | 1.268 | 0.587 | 0.871 | 0.377 |
| GSGQNIGHSLPQQT | 695 | 1.264 | 6.183 | 0.740 | 0.478 |
| GSGERSISVQNQQT | 696 | 1.264 | 1.619 | 0.598 | 0.173 |
| GSGLKPNVLQNQQT | 697 | 1.263 | 0.975 | 0.701 | 0.268 |
| GSGQVAYAQGNQQT | 698 | 1.259 | 1.309 | 0.734 | 0.313 |
| GSGQSSYGSGNQQT | 699 | 1.257 | 1.686 | 1.161 | 0.456 |
| GSGQNQAMTHGDQT | 700 | 1.257 | 1.878 | 0.357 | 0.259 |
| GSGQNQALVSMGQT | 701 | 1.255 | 1.876 | 0.987 | 0.560 |
| GSGQNPSFMRGQQT | 702 | 1.252 | 1.454 | 1.293 | 1.094 |
| GSGQNQQSHLRTNT | 703 | 1.251 | 4.583 | 1.022 | 0.718 |
| GYTRLETSGQNQQT | 704 | 1.250 | 1.323 | 0.841 | 0.297 |
| GSGQSYDMRGNQQT | 705 | 1.248 | 0.567 | 0.588 | 0.368 |
| GSRTTQDIGQNQQT | 706 | 1.247 | 0.000 | 0.685 | 0.280 |
| GSGHPYKAAQNQQT | 707 | 1.246 | 0.000 | 0.872 | 0.507 |
| GRLSNAHGGQNQQT | 708 | 1.245 | 0.839 | 1.036 | 0.725 |
| GSGQNQRAVLNDQT | 709 | 1.242 | 3.023 | 0.556 | 0.259 |
| GGSHTYGGGQNQQT | 710 | 1.241 | 13.065 | 0.982 | 0.730 |
| GSSVNSMIGQNQQT | 711 | 1.239 | 0.000 | 0.976 | 0.580 |
| GNSSMMGSGQNQQT | 712 | 1.239 | 3.856 | 0.656 | 0.364 |
| GNRDRPSSGQNQQT | 713 | 1.239 | 3.947 | 0.298 | 0.178 |
| GSGNMHASRQNQQT | 714 | 1.238 | 3.878 | 0.782 | 0.687 |
| GFIFPKVSGQNQQT | 715 | 1.237 | 0.000 | 1.764 | 0.692 |
| GSGQNQQLKNSTST | 716 | 1.235 | 1.703 | 1.063 | 0.538 |
| GSGQNQQSQYMPRT | 717 | 1.234 | 0.401 | 0.549 | 0.520 |
| GSGQRMADIGNQQT | 718 | 1.233 | 2.539 | 0.352 | 0.427 |
| GSGQNQSHYPSQQT | 719 | 1.228 | 4.315 | 0.644 | 0.402 |
| GSDGKMHRGQNQQT | 720 | 1.227 | 0.000 | 1.826 | 0.776 |
| GSGSVGFIGQNQQT | 721 | 1.227 | 8.261 | 0.689 | 0.445 |
| GLHGMTLSGQNQQT | 722 | 1.226 | 3.552 | 0.470 | 0.338 |
| GSDQSKRGDSNQQT | 723 | 1.225 | 0.639 | 0.479 | 0.267 |
| GSLFLATGGQNQQT | 724 | 1.220 | 0.000 | 0.775 | 0.485 |
| GSGQNQQPSAFSKT | 725 | 1.220 | 4.906 | 1.309 | 0.754 |
| GSGQLPQSGLNQQT | 726 | 1.218 | 1.504 | 0.641 | 0.318 |
| GSGSKQNALQNQQT | 727 | 1.216 | 2.010 | 0.941 | 0.594 |
| GSGQRRELSQNQQT | 728 | 1.215 | 1.791 | 0.622 | 0.396 |
| GSGQREPKASNQQT | 729 | 1.214 | 2.793 | 0.399 | 0.520 |
| GSGQNQQHPSTQQT | 730 | 1.205 | 1.552 | 1.017 | 0.680 |
| GSQSTLGLGQNQQT | 731 | 1.204 | 3.246 | 0.594 | 0.400 |
| GSGQNQQMPGLSST | 732 | 1.204 | 1.887 | 0.234 | 0.181 |
| GSGQNQQTVGGKNL | 733 | 1.203 | 0.128 | 0.777 | 1.051 |
| GSSREFHSGQNQQT | 734 | 1.203 | 1.591 | 0.688 | 0.474 |
| GSGQNQQTVPSNLV | 735 | 1.201 | 0.791 | 0.434 | 0.281 |
| GSGQNAYSSQAQQT | 736 | 1.201 | 12.096 | 0.629 | 0.216 |
| GSGQNKDHSTRRQT | 737 | 1.197 | 0.000 | 0.384 | 0.477 |
| GQLGSVGSGQDQQT | 738 | 1.196 | 0.000 | 1.020 | 0.437 |
| GSGQHAAPGHNQQT | 739 | 1.195 | 5.999 | 0.600 | 0.199 |
| GSGQNQQTSQSPPT | 740 | 1.194 | 1.208 | 0.851 | 0.478 |
| GSGNYRDHEQNQQT | 741 | 1.193 | 7.389 | 0.287 | 0.222 |
| GSGQHSNQHVNQQT | 742 | 1.192 | 1.453 | 0.955 | 0.558 |
| GSGQTARNGINQQT | 743 | 1.192 | 2.030 | 1.002 | 0.472 |
| GSGQNQQHYGSQGT | 744 | 1.189 | 0.453 | 1.345 | 0.379 |
| GSGSPQASRQNQQT | 745 | 1.189 | 6.782 | 0.923 | 0.542 |
| GSGFSHSMGKNQQT | 746 | 1.188 | 9.809 | 1.381 | 0.611 |
| GSGQSHSLETNQQT | 747 | 1.188 | 1.319 | 0.520 | 0.363 |
| GTEQTRQSGQNQQT | 748 | 1.188 | 0.132 | 0.756 | 0.756 |
| GSGRHLASVQNQQT | 749 | 1.187 | 1.024 | 0.654 | 0.606 |
| GLGSKNHSGQNQQT | 750 | 1.187 | 5.046 | 0.825 | 0.224 |
| GSGQNQQTSHFPSA | 751 | 1.185 | 0.325 | 0.969 | 0.907 |
| GSGQLSGTPQNQQT | 752 | 1.185 | 1.382 | 1.025 | 0.643 |
| GSGQNQQAPHKKET | 753 | 1.180 | 0.598 | 0.994 | 0.689 |
| GSGQNQQTLRGSLE | 754 | 1.179 | 1.812 | 0.853 | 0.354 |
| GSIAMTSHGQNQQT | 755 | 1.178 | 1.435 | 0.551 | 0.438 |
| GSPGVSPSGQNQQT | 756 | 1.178 | 3.006 | 0.853 | 1.160 |
| GSGQNQQTGSSSRV | 757 | 1.176 | 0.580 | 0.995 | 1.128 |
| GSGQHLPLLGNQQT | 758 | 1.175 | 1.739 | 0.519 | 0.347 |
| GSDHSHRGGQNQQT | 759 | 1.174 | 0.504 | 0.818 | 0.331 |
| GSGIVTKLGQNQQT | 760 | 1.174 | 10.571 | 0.599 | 0.242 |
| GSGQDVTKTGNQQT | 761 | 1.173 | 4.523 | 0.531 | 0.035 |
| GSGQNQQSHGRIGT | 762 | 1.173 | 5.117 | 0.607 | 0.455 |
| GSGQNQQINHRSPT | 763 | 1.173 | 0.748 | 0.259 | 0.220 |
| GSGDDSRVGQNQQT | 764 | 1.172 | 0.191 | 0.466 | 0.156 |
| GSGQSTLKRINQQT | 765 | 1.168 | 13.442 | 0.534 | 1.184 |
| GSGSQHSKAQNQQT | 766 | 1.168 | 0.312 | 0.638 | 0.916 |
| GSGQNQQHASSNNT | 767 | 1.166 | 7.155 | 0.789 | 0.896 |
| GSRTYQVSGQNQQT | 768 | 1.164 | 1.853 | 0.638 | 0.641 |
| GSGQNQGLLSSPQT | 769 | 1.164 | 0.000 | 0.707 | 0.417 |
| GSGGGLQHNQNQQT | 770 | 1.163 | 4.098 | 1.137 | 0.778 |
| GSGQNQQTTAATRM | 771 | 1.163 | 3.925 | 0.947 | 1.005 |
| GSGQNQRASILVQT | 772 | 1.162 | 3.632 | 0.531 | 0.569 |
| GSGQNLGLLGAQQT | 773 | 1.161 | 1.458 | 0.524 | 0.226 |
| GSLDLGRSGQNQQT | 774 | 1.160 | 3.283 | 1.002 | 0.505 |
| GNSQVKVSGQNQQT | 775 | 1.158 | 4.930 | 1.422 | 0.728 |
| GSSGSHQYGQNQQT | 776 | 1.155 | 0.000 | 1.129 | 0.794 |
| GSGQNQQQRDGTLT | 777 | 1.152 | 0.387 | 0.760 | 0.730 |
| GRGQHVSVANNQQT | 778 | 1.152 | 1.896 | 1.032 | 0.589 |
| GDSSSRISGQNQQT | 779 | 1.151 | 3.787 | 0.916 | 0.348 |
| GSGQNQQHSLSSQT | 780 | 1.150 | 3.844 | 0.700 | 0.730 |
| GSLMDVHRGQNQQT | 781 | 1.150 | 0.387 | 1.009 | 0.238 |
| GSIQYQSSGQNQQT | 782 | 1.147 | 2.601 | 1.074 | 1.191 |
| GLGSKNPSGQNQQT | 783 | 1.147 | 1.629 | 1.184 | 0.424 |
| GSGQLVLTLQNQQT | 784 | 1.143 | 0.000 | 0.336 | 0.336 |
| GSGQNQQTSQPLPG | 785 | 1.141 | 0.080 | 0.748 | 0.530 |
| GSGQNQQNLGKLNT | 786 | 1.141 | 0.000 | 0.919 | 0.687 |
| GTTAHQPSGQNQQT | 787 | 1.138 | 0.211 | 0.726 | 0.275 |
| GSGQNRAQIGTQQT | 788 | 1.138 | 0.469 | 0.776 | 0.654 |
| GSGQYVHVSSNQQT | 789 | 1.137 | 1.803 | 0.739 | 0.366 |
| GSGQNQQTAHAFNI | 790 | 1.132 | 3.404 | 0.699 | 0.729 |
| GSGQNQRTMVATQT | 791 | 1.130 | 1.122 | 0.649 | 0.554 |
| GSGQNPIRGAMQQT | 792 | 1.126 | 1.327 | 1.296 | 0.427 |
| GSGYVITGSQNQQT | 793 | 1.125 | 6.271 | 0.971 | 0.248 |
| GRGPKQSNIQNQQT | 794 | 1.125 | 0.737 | 0.771 | 2.490 |
| GSGQNQQTMLGKPC | 795 | 1.125 | 0.047 | 1.090 | 0.992 |
| GSGQNQQVGSTVRT | 796 | 1.124 | 2.040 | 0.918 | 0.614 |
| GNVTTQKSGQNQQT | 797 | 1.122 | 2.546 | 1.215 | 0.922 |
| GSGNPVSHLQNQQT | 798 | 1.121 | 1.037 | 0.583 | 0.310 |
| GSLSHMESGQNQQT | 799 | 1.120 | 0.829 | 0.489 | 0.265 |
| GRAPTNLSGQNQQT | 800 | 1.118 | 0.687 | 0.757 | 0.169 |
| GSGQNQQTVMTARA | 801 | 1.117 | 1.535 | 0.995 | 0.843 |
| GSGMPASRLQNQQT | 802 | 1.117 | 1.689 | 0.790 | 0.372 |
| GVVRNHQSGQNQQT | 803 | 1.116 | 5.801 | 0.899 | 0.868 |
| GSGQNQHSVQVRQT | 804 | 1.116 | 1.909 | 0.782 | 0.916 |
| GSGQNTGHLTMQQT | 805 | 1.114 | 0.078 | 1.026 | 0.595 |
| GSGQNQQYAGKILT | 806 | 1.112 | 0.300 | 1.078 | 0.431 |
| GSGNPHVRNQNQQT | 807 | 1.112 | 0.873 | 0.732 | 0.755 |
| GSGQNGGSSNRQQT | 808 | 1.109 | 2.594 | 1.255 | 0.844 |
| GSGQRLSQGVNHQT | 809 | 1.108 | 3.394 | 0.931 | 1.141 |
| GSGQNAHAKEGQQT | 810 | 1.108 | 0.000 | 0.875 | 1.179 |
| GSSPAPNSGQNQQT | 811 | 1.106 | 2.229 | 0.719 | 0.368 |
| GLAHKTSSGQNQQT | 812 | 1.106 | 0.915 | 0.427 | 0.690 |
| GSGQNQQTPGAHKT | 813 | 1.105 | 3.827 | 0.957 | 0.277 |
| GSGQNQQSLSGSFT | 814 | 1.105 | 0.735 | 0.745 | 0.883 |
| GSGQNQQSTGTSRT | 815 | 1.103 | 4.054 | 1.209 | 0.935 |
| GSGQNQQTVQSNLV | 816 | 1.103 | 2.350 | 0.577 | 0.698 |
| GSGQNQQLGSRQCT | 817 | 1.102 | 0.183 | 0.987 | 0.407 |
| GSGQNQYLRLELQT | 818 | 1.101 | 0.000 | 0.416 | 0.839 |
| GSGQNQQTSPRLQT | 819 | 1.100 | 0.795 | 1.156 | 1.091 |
| GSGQNQQTTSSNMT | 820 | 1.099 | 0.569 | 0.638 | 0.698 |
| GTASTYNSGQNQQT | 821 | 1.099 | 2.560 | 0.250 | 0.625 |
| GSGQNQQTMPQHKI | 822 | 1.097 | 2.394 | 0.479 | 0.197 |
| GSGQSHLHTGNQQT | 823 | 1.096 | 2.584 | 0.721 | 0.295 |
| GVKGVGHSGQNQQT | 824 | 1.096 | 2.485 | 0.994 | 0.783 |
| GSGKVTKQSQNQQT | 825 | 1.095 | 0.000 | 0.928 | 1.035 |
| GSGQNQQTALEKSL | 826 | 1.092 | 0.000 | 0.625 | 0.702 |
| GSGYKDTYGQNQQT | 827 | 1.091 | 0.854 | 0.717 | 0.448 |
| GSGQNQQSGTFLST | 828 | 1.090 | 5.673 | 1.021 | 0.742 |
| GSGQNTGQHMMQQT | 829 | 1.090 | 1.058 | 1.147 | 0.917 |
| GSGKNQQRPGLDQT | 830 | 1.089 | 1.557 | 0.583 | 0.385 |
| GSGQSREISLNQQT | 831 | 1.088 | 6.954 | 0.594 | 0.282 |
| GTPTSPSSGQNQQT | 832 | 1.086 | 4.558 | 0.833 | 0.662 |
| GKPAGGLSGQNQQT | 833 | 1.085 | 2.805 | 0.708 | 0.739 |
| GSGQNHRSADMQQT | 834 | 1.084 | 12.001 | 0.417 | 0.212 |
| GSGQNQQTLPSLSL | 835 | 1.084 | 1.758 | 0.527 | 0.175 |
| GSPYMGATGQNQQT | 836 | 1.083 | 5.364 | 0.918 | 0.254 |
| GSGHAKAVGQNQQT | 837 | 1.081 | 4.357 | 0.703 | 0.824 |
| GHMKGVTSGQNQQT | 838 | 1.081 | 2.814 | 0.807 | 0.413 |
| GSGQNQKILTLDQT | 839 | 1.080 | 0.371 | 0.291 | 0.314 |
| GSGQNQQTKVGHSA | 840 | 1.079 | 1.256 | 0.669 | 1.019 |
| GIARTTISGQNQQT | 841 | 1.078 | 1.783 | 0.819 | 0.330 |
| GSGQNQQTSVGERT | 842 | 1.077 | 3.737 | 0.648 | 0.534 |
| GSGQNQQTMIANIR | 843 | 1.076 | 0.000 | 0.379 | 0.458 |
| GDMTRSSSGQNQQT | 844 | 1.075 | 0.802 | 1.145 | 1.038 |
| GSGHMSDLRQNQQT | 845 | 1.073 | 4.291 | 0.555 | 0.328 |
| GRGAVMASGQNQQT | 846 | 1.072 | 0.923 | 0.783 | 0.605 |
| GSGQNQQLSGKSVT | 847 | 1.070 | 1.524 | 1.276 | 0.930 |
| GSHTLVVSGQNQQT | 848 | 1.069 | 1.535 | 0.671 | 0.748 |
| GSGPWSAGLQNQQT | 849 | 1.067 | 0.947 | 0.700 | 0.539 |
| GSGQHSPHALNQQT | 850 | 1.064 | 1.412 | 0.885 | 0.573 |
| GSGQNQQPNSGSMT | 851 | 1.064 | 0.925 | 0.588 | 0.339 |
| GSGLAHLGGQNQQT | 852 | 1.064 | 2.191 | 0.749 | 0.794 |
| GSSVRYEPKQNQQT | 853 | 1.063 | 1.564 | 0.450 | 0.501 |
| GSGQNQQARPLELT | 854 | 1.061 | 0.059 | 0.389 | 0.252 |
| GSGQPRSTGINQQT | 855 | 1.061 | 0.693 | 0.650 | 0.542 |
| GSGQNQANWVKVQT | 856 | 1.059 | 0.126 | 0.683 | 0.532 |
| GSGHLFQSGQNQQT | 857 | 1.057 | 0.615 | 0.751 | 0.386 |
| GSGQNRGISISQQT | 858 | 1.057 | 2.166 | 0.686 | 0.566 |
| GSGTHYDNRQNQQT | 859 | 1.054 | 0.072 | 0.612 | 0.486 |
| GSGQNQQTSTTPLP | 860 | 1.052 | 2.823 | 0.828 | 0.741 |
| GSGQVHASQVNQKT | 861 | 1.049 | 0.503 | 0.855 | 0.767 |
| GSSGHRESGQNQQT | 862 | 1.048 | 4.398 | 0.641 | 0.691 |
| GLSAEKSSGQNQQT | 863 | 1.047 | 7.203 | 0.629 | 0.303 |
| GSGQEHRSLANQQT | 864 | 1.046 | 0.000 | 0.507 | 0.344 |
| GSGQTVVRIANQQT | 865 | 1.046 | 4.156 | 0.661 | 0.390 |
| GSGQNVSSVHRQQT | 866 | 1.045 | 0.712 | 0.383 | 0.271 |
| GSGASRMSIQNQQT | 867 | 1.045 | 0.111 | 0.801 | 0.417 |
| GVAFIGSSGQNQQT | 868 | 1.043 | 0.000 | 0.744 | 0.648 |
| GSGQNQQTVPTRQT | 869 | 1.040 | 1.207 | 0.629 | 0.138 |
| GSGQAAKSSQNQQT | 870 | 1.036 | 0.681 | 0.778 | 0.737 |
| GSGQNQQVAIRTST | 871 | 1.035 | 2.447 | 0.963 | 0.370 |
| GSVHMQNAGQNQQT | 872 | 1.034 | 3.608 | 1.004 | 0.625 |
| GSGMRQAGVQNQQT | 873 | 1.032 | 0.811 | 0.736 | 0.775 |
| GSGQNQQVGGKTVT | 874 | 1.032 | 6.195 | 1.094 | 0.821 |
| GVHDMRVSGQNQQT | 875 | 1.032 | 8.083 | 1.171 | 0.818 |
| GSGQHVSVANNQQT | 876 | 1.029 | 5.734 | 0.974 | 0.577 |
| GSAAMSVRGQNQQT | 877 | 1.029 | 2.386 | 0.202 | 0.287 |
| GVSRGGPSGQNQQT | 878 | 1.028 | 1.611 | 0.750 | 0.591 |
| GSGQMVHTIGNQQT | 879 | 1.026 | 1.328 | 0.406 | 0.430 |
| GRGGSMAETQNQQT | 880 | 1.024 | 2.853 | 0.799 | 0.669 |
| GSGHTNPTRQNQQT | 881 | 1.021 | 0.688 | 0.726 | 0.807 |
| GSGEAARYEQNQQT | 882 | 1.020 | 0.000 | 0.107 | 0.125 |
| GSGQNERHLVLQQT | 883 | 1.019 | 5.354 | 0.416 | 0.150 |
| GSGQNQQSKQQVLT | 884 | 1.019 | 1.494 | 1.428 | 1.256 |
| GSGQARAHRGNQQT | 885 | 1.017 | 0.000 | 0.254 | 0.386 |
| GSGQNQQPLDTSRT | 886 | 1.015 | 0.775 | 0.491 | 0.376 |
| GSGQNQQLANMVTT | 887 | 1.014 | 1.739 | 1.253 | 0.987 |
| GSGQMKDLHRNQQT | 888 | 1.014 | 1.068 | 0.587 | 0.506 |
| GSGQNQHLSSFVQT | 889 | 1.013 | 0.110 | 1.090 | 0.364 |
| GSGQNQQPSSRVTT | 890 | 1.012 | 2.179 | 0.784 | 0.504 |
| GSGQNQQLAITLGT | 891 | 1.011 | 0.000 | 0.877 | 0.143 |
| GSGQNQQTVGNPAT | 892 | 1.008 | 3.014 | 0.856 | 0.395 |
| GSGQNQGRAHPMQT | 893 | 1.007 | 2.364 | 0.684 | 0.453 |
| GSGQLIASVVNQQT | 894 | 1.005 | 0.086 | 0.197 | 0.359 |
| GSSVRSLVGQNQQT | 895 | 1.004 | 3.840 | 0.412 | 0.608 |
| GGAGSAHSGQNQQT | 896 | 1.003 | 6.108 | 0.474 | 1.092 |
| GSDQNQQTMSSTRT | 897 | 1.003 | 2.428 | 1.306 | 0.835 |
| GSGQNQQMAGAFRT | 898 | 1.003 | 1.784 | 1.307 | 0.762 |
| GSLGNLQRGQNQQT | 899 | 1.003 | 0.895 | 0.947 | 0.385 |
| GSGPSISHGQNQQT | 900 | 1.000 | 0.000 | 0.614 | 0.665 |
| GSGQNQQT | 6406 | 1.000 | 1.000 | 1.000 | 1.000 |
| GSGQNQQSSFNVQT | 901 | 0.998 | 0.000 | 1.307 | 0.675 |
| GSGQNQQTGQATHN | 902 | 0.996 | 2.199 | 0.877 | 0.527 |

A second cross-species evolution screen was performed using an AAV capsid variant library with a modification in loop IV introduced as described above and passaging it once through NHPs (passage 1) and then subsequently injected it into two different strains of mice (passage 2), C57Bl/6 and BALB/c. The fold-enrichment for each variant in the brain of each mouse species was calculated by systematic NGS enrichment analysis following RNA recovery and RT-PCR amplification. The fold enrichment values in the second passage in mice were compared to those fold enrichment values from the second pass that was performed in NHPs as described above. As shown in Table 10, when comparing the second pass fold enrichment values in the mice versus NHPs, 12 variants were identified that had a fold-enrichment value greater than 10 in all three animal groups. Further, 10 of these 12 variants comprised the SPH motif and a positive residue in one of the next three subsequent residues (Table 10).

TABLE 10
NGS fold-enrichment of AAV capsid variants from a
second passage (P2) in NHPs or mice (C57Bl/6 or BALB/c)
following a first passage in NHPs

| | | Fold | Fold | Fold |
|---|---|---|---|---|
| | | enrichment | enrichment | enrichment |
| Peptide | SEQ | over AAV9 | over AAV9 | over AAV9 |
| Sequence | ID NO: | in NHP P2 | in BALB/c P2 | in C57Bl/6 P2 |
| VSGSPHSKAQNQQT | 903 | 99.76 | 92.99 | 34.29 |
| CSGSPHSKAQNQQT | 904 | 85.1 | 66.74 | 22.19 |
| GSGSPHSKAQNQQT | 200 | 56.33 | 44.58 | 14.48 |
| GSGSPHRKAQNQQT | 905 | 46.39 | 42.47 | 14.11 |
| GRGSPHSKAQNQQT | 906 | 43.68 | 59.65 | 28.13 |
| GHDSPHKSGQNQQT | 201 | 33.96 | 59.14 | 27.15 |
| GSGSPHSKAKNQQT | 208 | 31.27 | 41.51 | 14 |
| GSGSPHSKAQNKQT | 907 | 29.52 | 44.1 | 13.69 |
| GSGSPHSKAQTQQT | 908 | 24.27 | 41.75 | 18 |
| GQDSPHKSGQNQQT | 204 | 22.7 | 32.37 | 16.02 |
| GSGSTHASRQNQQT | 909 | 11.04 | 23.71 | 10.67 |
| GHDSQHKSGQNQQT | 404 | 10.36 | 21.3 | 13.55 |

Following the second passage in mice, a synthetic library was generated using those variants that demonstrated a fold-change in enrichment relative to wild-type AAV9 that was above 10 in the brain of either strain of mice, as measured by systematic NGS enrichment analysis following RNA recovery and RT-PCR amplification. There were approximately 500 variants in this synthetic library. This synthetic library was then injected back into both strains of mice (C57Bl/6 and BALB/c; passage 3). RNA was recovered from the mouse brains, RT-PCR amplification was performed, and fold-enrichment relative to wild-type AAV9 was calculated by NGS analysis, which is provided in Table 11. As shown in Table 11, the variants with the greatest fold-enrichment in the brain in each strain, were highly correlated across strains (R²=0.8458).

TABLE 11
NGS fold-enrichment of AAV capsid variants in the
brain from a third passage (P3) in mice (C57Bl/6 or BALB/c)
following a first and second passage in mice

| | | Fold | Fold | |
|---|---|---|---|---|
| | | enrichment | enrichment | |
| Peptide | SEQ | over AAV9 | over AAV9 | |
| Sequence | ID NO: | in BALB/c | in C57Bl/6 | Average |
| GSGSPHKYGQNQQT | 910 | 150.445 | 103.488 | 126.966 |
| GSGSPHKFGQNQQT | 911 | 73.364 | 60.304 | 66.834 |
| GHDSPHKSGQNQQT | 201 | 82.460 | 51.125 | 66.792 |
| GSGSPHSKAQNQQT | 200 | 60.312 | 65.853 | 63.083 |
| VSGSPHKFGQNQQT | 912 | 60.186 | 59.142 | 59.664 |
| GSGSPHSKAQNHQT | 913 | 63.486 | 51.647 | 57.566 |
| VSGSPHSKAQNQQT | 903 | 73.555 | 37.429 | 55.492 |
| GQDSPHKSGQNQQT | 204 | 63.898 | 43.752 | 53.825 |
| GSGSPHSKAQHQQT | 914 | 45.309 | 45.600 | 45.454 |
| GSGSPHKTYQNQQT | 915 | 50.283 | 35.460 | 42.871 |
| GSGSPHSKAQTQQT | 908 | 43.120 | 39.098 | 41.109 |
| VSGSPHASRQNQQT | 916 | 46.572 | 32.480 | 39.526 |
| GSGSPHSKAQNKQT | 907 | 39.848 | 35.596 | 37.722 |
| GSGSPHKFGKNQQT | 917 | 31.948 | 34.899 | 33.423 |
| GSGSPHASRQNQHT | 918 | 28.145 | 30.928 | 29.537 |
| GSHSPHKSGQNQQT | 919 | 22.948 | 35.412 | 29.180 |
| GSGQNQQRRMSPST | 920 | 4.576 | 53.520 | 29.048 |
| GSGSPHASRQNQQT | 205 | 28.866 | 29.139 | 29.003 |
| GSGSPHSKPQNQQT | 921 | 26.958 | 28.599 | 27.779 |
| GSGSPHKFGQKQQT | 922 | 39.597 | 14.927 | 27.262 |
| VSGSPHGARQNQQT | 923 | 30.985 | 22.634 | 26.810 |
| GSGSPHSKAQKQQT | 924 | 25.052 | 27.459 | 26.256 |
| GHSSPHRSGQNQQT | 212 | 16.982 | 35.081 | 26.032 |
| GSGSPHSKAKNQQT | 208 | 21.069 | 25.711 | 23.390 |
| GSHSPHKRGQNQQT | 925 | 24.054 | 20.262 | 22.158 |
| GRGSPHSKAQNQQT | 906 | 20.939 | 22.720 | 21.830 |
| GQSSPHRSGQNQQT | 229 | 9.916 | 26.608 | 18.262 |
| GSGQNRQRLKGLET | 926 | 3.937 | 31.022 | 17.480 |
| GSGSPHKLGQNQQT | 927 | 18.905 | 14.732 | 16.818 |
| GSGSPHKTSKNQQT | 928 | 14.654 | 17.606 | 16.130 |
| GSGSPHKIGQNQQT | 929 | 16.999 | 14.794 | 15.897 |
| GSGSPHKKNQNQQT | 209 | 25.633 | 5.605 | 15.619 |
| GSGSPHASRQNKQT | 206 | 10.738 | 20.347 | 15.542 |
| GSGSPHTRGQNQQT | 214 | 16.899 | 13.869 | 15.384 |
| GSGQDSPHVRNQQT | 930 | 15.340 | 14.646 | 14.993 |
| GSGSPHKTSQNQQT | 931 | 20.428 | 8.818 | 14.623 |
| GSGSPHASRKNQQT | 932 | 13.799 | 12.749 | 13.274 |
| GSGSPHASRQKQQT | 211 | 13.624 | 11.188 | 12.406 |
| GSHSPHKSGQKQQT | 933 | 6.700 | 17.736 | 12.218 |
| GSGSPHKTSQKQQT | 934 | 12.621 | 11.720 | 12.170 |
| GSGSPHVRGQNKQT | 935 | 13.174 | 11.017 | 12.095 |
| GSGSPHKTTQNQQT | 936 | 9.722 | 13.381 | 11.552 |
| CSGSPHSKAQNQQT | 904 | 11.772 | 9.447 | 10.610 |
| GSGPVRALRQNQQT | 937 | 3.369 | 17.431 | 10.400 |
| GSGSPHVRGQKQQT | 938 | 7.573 | 12.498 | 10.036 |
| GSGSPHRKAQNQQT | 905 | 12.308 | 7.349 | 9.828 |
| GRGSPHASRQNQQT | 318 | 11.903 | 6.780 | 9.342 |
| CSGSPHKTSQNQQT | 939 | 11.167 | 6.631 | 8.899 |
| CSHSPHKSGQNQQT | 940 | 11.356 | 6.304 | 8.830 |
| GSGSPHSKDQNQQT | 604 | 3.492 | 10.236 | 6.864 |

Taken together, these results demonstrate that after 3 rounds of screening of this AAV9 variant library with loop IV modifications in NHP and mice, many AAV capsid variants outperformed the wild-type AAV9, for example, in penetration of the blood brain barrier (BBB) and spinal cord expression. These capsid variants were able to cross-species, evidenced by expression and tropism in the NHP brain/spinal cord as well as in the brain of two different mouse species.

### Example 11. Individual Capsid Characterization in Mice

The goal of these experiments was to determine the transduction level, tropism, ability to cross the blood brain barrier, and overall spatial distribution in the central nervous system (CNS) of 2 capsid variants selected from the study described in Example 1 relative to AAV9 following intravenous injection in mice. The 2 capsid variants were TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2), as outlined in Table 3 above. The amino acid and DNA sequences of TTM-001 and TTM-002 are provided. e.g., in Tables 4 and 5, respectively.

AAV particles were generated with each of these capsid variants encapsulating a luciferase-EGFP transgene driven by a CMV/chicken beta actin promoter in a single stranded viral genome. Each capsid variant and AAV9 control were tested by intravenously administering by tail vein injection, the AAV particle formulation at 5e11 VG/dose (2.5E13 vg/kg) to three female BALB/c mice. The in-life period was 28 days and then various CNS and peripheral tissues were collected for measuring transgene mRNA, transgene protein, and viral DNA (biodistribution).

At 28 days post-injection of the AAV particles encapsulated in the TTM-001 capsid variant (AAV_TTM-001), mice were injected with luciferin and their brains were harvested for IVIS imaging. Robust luciferase signal was observed in mice injected with AAV particles encapsulated in the TTM-001 capsid variant, and this was greatly increased relative to AAV particles encapsulated in the wild-type AAV9 control capsid.

The brains isolated from mice injected with the AAV particles encapsulated in the TTM-001 capsid variant (AAV_TTM-001) or the TTM-002 capsid variant (AAV_TTM-002) were assayed by qPCR for the presence of transgene RNA as a measure of transgene expression, and the presence of viral DNA as a measure of viral genome levels. Data were provided as fold over AAV9 (Table 12). As shown in Table 12, when compared to the wild-type AAV9 capsid control, TTM-001 and TTM-002 demonstrated a 30-fold and 66-fold increase, respectively, in transgene mRNA levels and expression in the brain, indicative of enhanced payload delivery. This correlated with a 32-fold (TTM-001) and 47-fold (TTM-002) increase, respectively, in viral genome (DNA) concentrations in the brain relative to the AAV9 capsid control, which is indicative of enhanced CNS tropism and transduction (Table 12).

TABLE 12
Transgene mRNA and viral genome levels (DNA)
in mice relative to the AAV9 control

| Measure | Tissue | AAV9 | TTM-001 | TTM-002 |
|---|---|---|---|---|
| mRNA | Brain | 1.0 | 30.4503 | 66.2161 |
| (transgene expression) | | | | |
| DNA | Brain | 1.0 | 32.0315 | 47.2810 |
| (viral genome quantification) | | | | |
| mRNA | Liver | 1.0 | 1.2356 | 0.2016 |
| (transgene expression) | | | | |
| DNA | Liver | 1.0 | 0.4802 | 0.0277 |
| (viral genome quantification) | | | | |

The brain tissues and spinal cords of the mice were also subjected to anti-GFP immunohistochemistry staining to evaluate overall CNS tropism and biodistribution. Immunohistochemical staining correlated with the qPCR analysis, as TTM-001 and TTM-002 showed significantly stronger staining and payload expression in the brain and spinal cord, as compared to the AAV9 control. More specifically, TTM-001 and TTM-02 demonstrated localization and strong payload expression and transduction in the mid-brain region, with increased staining observed in the hippocampus and thalamus, as well as in the brain stem, compared to AAV9. Less staining was observed in the cortical regions of the brain compared to the midbrain. However, staining in these cortical regions was stronger for TTM-001 and TTM-002 compared to the AAV9 control. It also appeared that the TTM-001 and TTM-002 capsid variants were able to transduce non-neuronal cells, including glial cells and oligodendrocytes. With respect to the spinal cord, staining and payload expression for TTM-01 and TTM-002 were localized to the ventral horns of the grey matter.

Peripheral tissues were also isolated from the mice intravenously injected with the AAAV particles encapsulated in the TTM-001 capsid variant or the TTM-002 capsid variant for analysis by qPCR and/or GFP immunohistochemical staining. Transgene mRNA levels and viral genome DNA levels were quantified in the liver by qPCR and the fold over AAV9 was calculated for each capsid variant (Table 12). TTM-001 resulted in similar levels of payload expression (mRNA levels) as compared to wild-type AAV9, but only half as much viral genome DNA was quantified in the liver compared to AAV9. TTM-002 demonstrated greatly reduced mRNA and viral genome DNA levels in the liver compared to AAV9. GFP immunohistochemical staining of the spleen, heart, skeletal muscle, kidneys, and lungs of mice injected with AAV particles encapsulated in the TTM-001 capsid variant or the TTM-002 capsid variant showed similar levels of payload expression as compared to those mice injected with AAV particles encapsulated in the wild-type AAV9 control capsid.

Taken together, these data demonstrate that TTM-001 and TTM-002 are enhanced CNS tropic capsids in mice that can infect non-neuronal cells. Additionally, these capsid variants were able to successfully penetrate the blood brain barrier following intravenous injection.

### Example 12. Maturation of TTM-001 and TTM-002 Capsid in NHPs

This Example describes maturation of the AAV9 capsid variants, TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2) in NHPs to further enhance their transduction and biodistribution in the central nervous system as well as other tissues, and evolve the AAV capsid variants to provide further cross-species compatibility. Two approaches were used to mature the TTM-001 and TTM-002 capsid sequences in order to randomize and mutate within and around the peptide insert comprised within loop IV of the capsid variant. As many of the AAV capsid variants that demonstrated the greatest fold-enrichment in the NHP brain relative wild-type AAV9 comprised an SPH motif in the same position (e.g., immediately subsequent to position 455, relative to a reference sequence numbered according to the amino acid sequence of SEQ ID NO: 138) (see Example 10), the SPH motif was not mutated in either approach to mature the TTM-001 and TTM-002 capsid variants. In the first maturation approach, sets of three contiguous amino acids were randomized across the mutagenesis region in the TTM-001 and TTM-002 sequences, which spanned from position 450 to position 466, numbered according to SEQ ID NO: 981 and 982. In the second maturation approach, mutagenic primers were used to introduce point mutations at a low frequency, scattered across the mutagenesis region in the TTM-001 and TTM-002 sequences ranging from position 449 to position 466, numbered according to SEQ ID NO: 981 and 982. AAV capsid variants arising from each maturation approach for TTM-001 and TTM-002 were pooled together, for subsequent testing and characterization in NHPs.

The library of pooled matured AAV capsid variants generated using the first maturation approach and the second maturation approach for the TTM-001 and TTM-002 AAV capsid variants were injected into two NHPs. After a period in life, the brains, heart, liver, muscle, and DRG of the NHPs were isolated and RNA was extracted. Following RNA recovery and RT-PCR amplification, a systematic NGS enrichment analysis was performed to calculate the fold enrichment ratio relative to an AAV9 control, and the peptides comprised within the variants were identified.

Following the RNA recovery and NGS analysis from the second maturation approach, approximately 680,000 capsid variants were identified. The 680,000 matured capsid variants were then filtered based on samples with a raw virus count greater than 10 and a coefficient of variance (CV) of less than 1, which was calculated for each peptide across the brain samples taken from the two NHPs. Those that had a CV value<1 were identified, as these were the peptides that were reliably detected in the majority of samples isolated from the brains of the two NHPs. Using this filtering criteria, this led to approximately 64,000 matured capsid variants.

Table 15 provides the peptide sequences of the matured capsid variants having a raw virus count greater than 10, a CV of less than 1 for the brain samples isolated, and that also demonstrated a 50-fold or greater fold-increase in expression in the brain relative to the AAV9 control in both mice and NHPs. The matured variants in Table 15, were also those variants that had a fold-change in expression that was less than 2 relative to the AAV9 control in the liver and the DRG. Applying these criteria, approximately 350 matured capsid variants were identified that demonstrated high transduction in the brain in NHPs and mice, cross-species compatibility in mice and NHPs, and were de-targeted in the liver and DRG, relative to the AAV9 control. Several variants as shown in Table 15, led to greater than 100-fold increase in expression relative to AAV9 in the NHP and/or mouse brain, with one variant resulting in a greater than 200-fold increase in expression relative to AAV9 in both species.

Fold-change in expression for the TTM-001 and TTM-002 matured variants in Table 15 that showed increased expression in the brain of the NHPs and mice, were also calculated for the DRG, muscle, liver (RNA and DNA), and heart of the NHPs following each maturation approach. As shown in Table 15, many variants were de-targeted in the peripheral tissues with a lower fold-change in expression relative to the AAV9 control, demonstrating CNS-specific tropism and a preferential transduction of the brain and CNS. Some variants demonstrated increased expression to AAV9 in multiple tissues, including the brain and peripheral tissues, demonstrating pan-tropism.

TABLE 15
NGS fold-enrichment of TTM-001 and TTM-002 matured
AAV capsid variants in the brain of NHPs and mice

| | | Fold Enrichment relative to AAV9 | | | | | | |
|---|---|---|---|---|---|---|---|---|
| | SEQ | | | | | Liver | Liver | |
| | ID | Brain | DRG | Heart | Muscle | RNA | DNA | Brain |
| Sequence | NO: | (NHP) | (NHP) | (NHP) | (NHP) | (NHP) | (NHP) | (Mouse) |
| KTIIGSGSPHSKAQNRHT | 3239 | 217.176 | 0.000 | 0.000 | 0.000 | 0.000 | 0.000 | 210.515 |
| KTFPGSGSPHSKVQNQQT | 3240 | 199.720 | 0.000 | 0.000 | 0.000 | 0.000 | 0.967 | 97.703 |
| KTEKMSGSPHSKAQNQQT | 3241 | 169.461 | 0.523 | 0.000 | 0.000 | 0.000 | 0.158 | 109.161 |
| KEINGRGSPHSKAQNQQT | 3527 | 134.390 | 0.239 | 0.000 | 0.000 | 0.000 | 0.232 | 52.311 |
| KTVNRNGSPHSKAQNQQT | 3528 | 133.016 | 0.000 | 0.416 | 0.000 | 0.000 | 0.000 | 85.361 |
| KTVNGSGSPHSKARDQQT | 3242 | 124.789 | 0.123 | 0.039 | 0.312 | 0.569 | 0.454 | 132.137 |
| KTFNGSGSPHSKAPNLQT | 3243 | 121.436 | 0.000 | 0.167 | 0.000 | 0.000 | 0.015 | 168.920 |
| KTEKTSGSPHSKAQNQQT | 3244 | 120.337 | 0.000 | 0.355 | 0.000 | 0.000 | 0.119 | 101.467 |
| KTINGSGSPHSKAHVRQT | 3245 | 119.798 | 0.000 | 0.000 | 0.262 | 0.694 | 1.039 | 165.590 |
| KTVNGSGSPHSKAPNQHT | 3246 | 117.207 | 0.000 | 0.109 | 0.000 | 0.000 | 0.074 | 51.008 |
| KTEKISGSPHSKAQNQQT | 3247 | 116.603 | 0.000 | 0.000 | 0.000 | 0.000 | 0.426 | 102.978 |
| KTINGPGSPHSKAHNQQT | 3529 | 115.742 | 0.146 | 0.000 | 0.235 | 0.000 | 0.513 | 52.508 |
| KTVNGSGSPHSKTQSQQT | 3248 | 115.086 | 0.000 | 0.726 | 0.000 | 0.000 | 0.340 | 63.248 |
| TTINGSGSPHSKAQNQQT | 3249 | 114.856 | 1.340 | 14.856 | 0.827 | 1.281 | 0.957 | 72.058 |
| KSINESGSPHSKAQNQQI | 3250 | 113.833 | 0.000 | 0.000 | 0.000 | 0.000 | 0.000 | 67.649 |
| KTERTSGSPHSKAQNQQT | 3251 | 112.957 | 0.000 | 0.009 | 0.000 | 1.128 | 0.207 | 117.374 |
| KTINGSGSPHSKAQPAKT | 3252 | 111.472 | 0.331 | 0.000 | 1.089 | 0.044 | 1.796 | 215.275 |
| KTEKSSGSPHSKAQNQQT | 3253 | 107.470 | 0.000 | 0.016 | 0.014 | 0.977 | 0.179 | 100.177 |
| KTSYGNGSPHSKAQNQQT | 3530 | 105.937 | 0.000 | 0.000 | 0.000 | 0.000 | 0.114 | 105.894 |
| KTEKGSGSPHSKAQNQQT | 3254 | 105.614 | 0.053 | 0.031 | 0.000 | 0.586 | 0.169 | 84.653 |
| KTINGSGSPHSKSQTQQN | 3255 | 104.474 | 0.000 | 0.131 | 0.000 | 0.084 | 0.038 | 54.021 |
| KTERISGSPHSKAQNQQT | 3256 | 103.692 | 0.000 | 0.000 | 0.000 | 0.062 | 0.370 | 89.637 |
| KTERASGSPHSKAQNQQT | 3257 | 103.669 | 0.000 | 0.000 | 0.000 | 0.127 | 0.070 | 115.550 |
| KELHGSGSPHSKAQNQQT | 3258 | 102.680 | 0.000 | 0.000 | 0.000 | 1.634 | 0.592 | 96.554 |
| KAINGSGSPHSKAQNLAT | 3259 | 101.954 | 0.000 | 10.954 | 8.655 | 0.298 | 0.239 | 116.685 |
| KTVNGSGSPHSKSQNQLT | 3260 | 101.327 | 0.000 | 0.035 | 0.000 | 0.000 | 0.025 | 80.716 |
| KTERNSGSPHSKAQNQQT | 3261 | 99.892 | 0.000 | 0.000 | 0.000 | 0.000 | 0.107 | 87.392 |
| KSVNGNGSPHSKAQNQQT | 3531 | 99.385 | 0.000 | 1.329 | 0.000 | 0.359 | 0.079 | 51.016 |
| KTFNGSGSPHSKAQGQQT | 3262 | 99.253 | 0.000 | 0.208 | 0.000 | 0.128 | 0.099 | 81.459 |
| KTINGSGSPHGWVQNQQT | 3532 | 97.122 | 0.000 | 0.000 | 0.000 | 1.240 | 1.975 | 290.720 |
| KTERVSGSPHSKAQNQQT | 3263 | 96.943 | 0.000 | 0.000 | 0.000 | 0.000 | 0.144 | 135.438 |
| KTINGSGSPHSKALNRQS | 3264 | 96.843 | 0.136 | 0.532 | 0.000 | 0.042 | 0.178 | 55.945 |
| KTERLSGSPHSKAQNQQT | 3265 | 95.857 | 0.000 | 0.004 | 0.005 | 0.126 | 0.260 | 102.372 |
| KTDNGSGSPHSKAHNQQT | 3266 | 95.164 | 0.000 | 0.000 | 0.000 | 0.000 | 0.027 | 55.313 |
| KTFHGSGSPHSKTQNQQT | 3267 | 94.714 | 0.000 | 0.210 | 0.120 | 0.000 | 0.000 | 51.119 |
| KTINGGGSPHSKAQTQQI | 3533 | 92.345 | 0.000 | 0.000 | 0.000 | 0.000 | 0.023 | 54.199 |
| KTSNGSGSPHSKAQNPPT | 3268 | 91.528 | 0.000 | 0.000 | 0.000 | 0.000 | 0.039 | 51.541 |
| ETINGSGSPHSKAQNLQT | 3269 | 90.969 | 0.221 | 1.023 | 0.197 | 0.179 | 0.813 | 107.216 |
| KTVHGNGSPHSKAQNQQT | 3534 | 90.073 | 0.000 | 0.000 | 0.000 | 0.000 | 0.304 | 97.003 |
| NTINGSGSPHSKAQNQQT | 3270 | 90.017 | 1.712 | 1.261 | 1.171 | 0.923 | 0.540 | 55.179 |
| KTINGGGSPHSKAQNQQC | 3535 | 89.301 | 0.219 | 0.000 | 0.000 | 0.287 | 0.319 | 53.840 |
| KTENMSGSPHSKAQNQQT | 3271 | 89.247 | 0.000 | 0.000 | 0.000 | 0.000 | 0.260 | 130.568 |
| KTENVSGSPHSKAQNQQT | 3272 | 88.506 | 0.000 | 0.000 | 0.000 | 0.964 | 0.112 | 108.591 |
| KTSSGSGSPHSKAQYQQT | 3273 | 87.304 | 0.000 | 0.000 | 0.000 | 0.000 | 0.299 | 58.143 |
| KTIDGGGSPHSKAQNKQT | 3536 | 85.019 | 0.000 | 0.000 | 0.000 | 0.000 | 0.477 | 55.517 |
| KTEKVSGSPHSKAQNQQT | 3274 | 84.558 | 0.000 | 0.022 | 0.000 | 0.873 | 0.424 | 112.185 |
| KAINGSGSPHSKAQDQET | 3275 | 84.080 | 0.000 | 0.000 | 0.000 | 0.194 | 0.027 | 87.637 |
| KTCNKSGSPHSKAQNQQT | 3276 | 83.992 | 0.000 | 0.000 | 0.165 | 0.283 | 0.000 | 119.496 |
| KTINGGGSPHSKAQNQLI | 3537 | 83.881 | 0.000 | 0.000 | 0.000 | 0.046 | 0.387 | 78.383 |
| KNINGGGSPHSKAQNQQT | 3538 | 83.083 | 0.000 | 0.042 | 0.000 | 0.000 | 0.000 | 75.913 |
| KTEHLSGSPHSKAQNQQT | 3277 | 83.080 | 0.000 | 0.000 | 0.012 | 0.021 | 0.189 | 69.494 |
| KAEMGSGSPHSKAQNQQT | 3278 | 83.049 | 0.000 | 0.020 | 0.000 | 0.768 | 0.112 | 135.019 |
| KATNGSGSPHSKAQNHQT | 3279 | 82.627 | 0.000 | 0.176 | 0.000 | 0.155 | 0.057 | 66.207 |
| KAIKGSGSPHSKAQDQQT | 3280 | 82.258 | 0.000 | 0.000 | 0.000 | 0.108 | 0.000 | 85.178 |
| KTINGGGSPHSKSQNQLT | 3539 | 82.231 | 0.000 | 0.070 | 0.000 | 0.000 | 0.498 | 126.986 |
| KTVNGNGSPHSKAQNKQT | 3540 | 81.481 | 0.000 | 0.000 | 0.000 | 0.000 | 0.122 | 69.455 |
| KTINGSGSPHSKGHWQQT | 3281 | 81.434 | 0.000 | 0.000 | 0.000 | 0.000 | 1.011 | 65.252 |
| KTDKTSGSPHSKAQNQQT | 3282 | 81.430 | 0.000 | 0.000 | 0.000 | 1.362 | 0.291 | 169.515 |
| KTFKGSGSPHSKAPNQQT | 3283 | 80.890 | 0.000 | 0.000 | 0.000 | 0.000 | 0.017 | 71.144 |
| KTVNGSGSPHSKAQNQLI | 3284 | 80.509 | 0.000 | 0.000 | 0.000 | 0.000 | 0.166 | 71.156 |
| KTINGSGSPHSKRPEQQT | 3285 | 80.418 | 0.000 | 0.013 | 0.000 | 0.149 | 0.361 | 50.319 |
| KTINGSGSPHSKAQRTMT | 3286 | 80.388 | 0.000 | 0.022 | 0.170 | 1.812 | 1.025 | 100.248 |
| KTEKASGSPHSKAQNQQT | 3287 | 80.285 | 0.000 | 0.041 | 0.000 | 0.000 | 0.261 | 90.390 |
| KSDQGSGSPHSKAQNQQT | 3288 | 80.076 | 0.000 | 0.000 | 0.000 | 0.993 | 0.124 | 151.911 |
| KTEITSGSPHSKAQNQQT | 3289 | 79.620 | 0.000 | 0.163 | 0.000 | 0.332 | 0.074 | 76.686 |
| KTDKSSGSPHSKAQNQQT | 3290 | 79.470 | 0.055 | 0.012 | 0.000 | 1.437 | 0.367 | 141.351 |
| KTIDGSGSPHSKAQNQQH | 3291 | 79.090 | 0.000 | 0.000 | 0.000 | 0.136 | 0.049 | 57.914 |
| KTVNGNGSPHSKAQNQHT | 3541 | 78.849 | 0.000 | 0.000 | 0.000 | 0.000 | 0.045 | 54.086 |
| KNTNGSGSPHSKAQNQQT | 3292 | 78.445 | 0.000 | 0.000 | 0.000 | 0.571 | 0.177 | 89.719 |
| KTETHSGSPHSKAQNQQT | 3293 | 77.974 | 0.000 | 0.067 | 0.000 | 0.000 | 0.512 | 57.287 |
| KTINGGGSPHSKALNQQN | 3542 | 77.822 | 0.000 | 0.131 | 0.000 | 0.000 | 0.274 | 69.884 |
| KTINGSGSPHSKALHQHT | 3294 | 77.502 | 0.000 | 0.052 | 0.041 | 0.000 | 0.188 | 68.196 |
| KTINGTGSPHSKAQNHQI | 3543 | 77.089 | 0.171 | 0.000 | 0.000 | 0.000 | 0.166 | 54.281 |
| KTINGSGSPHSKAQHRIT | 3295 | 76.849 | 0.105 | 0.499 | 0.170 | 1.424 | 0.214 | 127.000 |
| KTINGSGSPHSKAQYIHT | 3296 | 76.170 | 0.000 | 0.014 | 0.033 | 1.523 | 0.168 | 59.649 |
| KTENISGSPHSKAQNQQT | 3297 | 76.072 | 0.000 | 0.000 | 0.000 | 0.115 | 0.132 | 83.118 |
| KTIIGGGSPHSKAHNQQT | 3544 | 75.872 | 0.000 | 0.050 | 0.000 | 0.000 | 0.235 | 65.492 |
| KTINGSGSPHSKAQKFET | 3298 | 75.788 | 0.000 | 0.000 | 0.028 | 0.108 | 0.093 | 65.588 |
| KTSNESGSPHSKAQNHQT | 3299 | 75.720 | 0.000 | 0.000 | 0.000 | 0.169 | 0.217 | 70.590 |
| KTINGSGSPHSKAQFPST | 3300 | 75.677 | 0.000 | 0.004 | 0.000 | 0.849 | 0.127 | 119.712 |
| KTERPSGSPHSKAQNQQT | 3301 | 75.669 | 0.000 | 0.029 | 0.000 | 0.000 | 0.156 | 73.894 |
| KTINGNGSPHSKAQNPLT | 3545 | 75.269 | 0.000 | 0.000 | 0.000 | 0.366 | 0.000 | 53.583 |
| KSIKGNGSPHSKAQNQQT | 3546 | 75.196 | 0.000 | 0.000 | 0.000 | 0.000 | 0.000 | 90.251 |
| KTERMSGSPHSKAQNQQT | 3302 | 74.910 | 0.000 | 0.000 | 0.000 | 0.100 | 0.151 | 122.812 |
| KTERSSGSPHSKAQNQQT | 3303 | 74.853 | 0.000 | 0.071 | 0.000 | 1.036 | 0.056 | 125.538 |
| KTELHSGSPHSKAQNQQT | 3304 | 74.620 | 0.000 | 0.000 | 0.000 | 0.021 | 0.089 | 53.124 |
| KTELTSGSPHSKAQNQQT | 3305 | 74.548 | 0.000 | 0.000 | 0.000 | 0.537 | 0.421 | 100.311 |
| KTINGSGSPHSKAHNQQR | 3306 | 74.272 | 0.562 | 0.486 | 0.047 | 0.956 | 0.057 | 107.301 |
| KTINGGGSPHSKAQSQQI | 3547 | 74.264 | 0.000 | 0.000 | 0.000 | 0.000 | 0.235 | 67.651 |
| KTINGSGSPHSKAQAIKT | 3307 | 74.261 | 0.255 | 0.000 | 0.000 | 0.186 | 0.132 | 73.560 |
| KTENTSGSPHSKAQNQQT | 3308 | 74.061 | 0.000 | 0.000 | 0.218 | 0.233 | 0.730 | 96.249 |
| KTIDGSGSPHSKGQNRQT | 3309 | 73.930 | 0.000 | 0.000 | 0.000 | 0.106 | 0.091 | 63.626 |
| KNINGSGSPHSKAQSQQT | 3310 | 73.757 | 0.000 | 0.000 | 0.000 | 0.000 | 0.041 | 57.432 |
| KTINGSVSPHGKAQNQLT | 3548 | 73.525 | 0.000 | 0.061 | 0.067 | 0.000 | 0.053 | 51.358 |
| KTSNASGSPHSKAQNQLT | 3311 | 73.501 | 0.000 | 0.000 | 0.297 | 0.000 | 0.313 | 150.401 |
| KTEARSGSPHSKAQNQQT | 3312 | 73.349 | 0.000 | 0.000 | 0.000 | 0.695 | 0.118 | 62.903 |
| KTEKNSGSPHSKAQNQQT | 3313 | 73.347 | 0.000 | 0.000 | 0.044 | 0.159 | 0.021 | 74.393 |
| KTANGSGSPHSKAQYQQT | 3314 | 73.038 | 0.000 | 0.000 | 0.000 | 0.153 | 0.160 | 139.451 |
| KTVNGSGSPHSKAQYQHT | 3315 | 72.847 | 0.000 | 0.000 | 0.000 | 0.000 | 0.130 | 54.158 |
| KTINGSGSPHTKAQNPQS | 3316 | 72.594 | 0.000 | 0.000 | 0.000 | 0.000 | 0.130 | 62.508 |
| KTINGSGSPHSKGQNPPT | 3317 | 72.339 | 0.000 | 0.206 | 0.000 | 0.000 | 0.041 | 134.808 |
| KTIIGSGSPHSKAQHQLT | 3318 | 72.291 | 0.000 | 0.000 | 0.000 | 0.000 | 0.000 | 100.144 |
| KTINGSGSPHSKAQSPPT | 3319 | 71.632 | 0.069 | 0.047 | 0.274 | 0.179 | 0.425 | 97.111 |
| NTIYGSGSPHSKAQNQQT | 3320 | 71.267 | 1.739 | 0.000 | 273.69 | 0.000 | 0.209 | 59.707 |
| KTINGSGSPHSKAQAKLT | 3321 | 71.154 | 0.000 | 0.273 | 0.017 | 1.591 | 0.777 | 130.132 |
| KTDKNSGSPHSKAQNQQT | 3322 | 70.964 | 0.000 | 0.000 | 0.000 | 0.070 | 0.123 | 62.932 |
| KTINGSGSPHSKTKSQQT | 3323 | 70.891 | 0.000 | 0.568 | 0.045 | 0.418 | 0.496 | 83.923 |
| KTINGSGSPHSKAQDRPT | 3324 | 70.831 | 0.132 | 0.006 | 0.000 | 0.039 | 0.379 | 66.800 |
| KTINGIGSPHSKAQNLGT | 3549 | 70.543 | 0.000 | 0.071 | 0.000 | 0.000 | 0.135 | 104.769 |
| KTINGSGSPHSKAQSQQL | 3325 | 70.539 | 0.000 | 0.000 | 0.000 | 0.000 | 0.041 | 51.126 |
| KTENLSGSPHSKAQNQQT | 3326 | 70.303 | 0.070 | 0.000 | 0.000 | 0.395 | 0.470 | 107.385 |
| KTINGSGSPHSKAQAFHT | 3327 | 70.159 | 0.033 | 0.000 | 0.058 | 0.762 | 0.119 | 86.268 |
| KTINGSGSPHSKAQKQQD | 3328 | 70.116 | 0.000 | 0.024 | 0.000 | 0.064 | 0.083 | 110.196 |
| KTFSGSGSPHSKAQNLQT | 3329 | 70.035 | 0.000 | 0.327 | 0.303 | 0.000 | 0.228 | 70.917 |
| KAINGSGSPHSKAQNAQT | 3330 | 69.651 | 0.000 | 0.000 | 0.000 | 0.023 | 0.142 | 72.160 |
| KTESWSGSPHSKAQNQQT | 3331 | 69.144 | 0.000 | 0.000 | 0.000 | 0.000 | 0.019 | 67.699 |
| KTTNGSGSPHSKAHNQLT | 3332 | 69.062 | 0.000 | 0.000 | 0.000 | 0.708 | 0.000 | 65.505 |
| KTVNGNGSPHSKAQNHQT | 3550 | 68.889 | 0.000 | 0.000 | 0.000 | 0.000 | 0.030 | 52.482 |
| KTEDKSGSPHSKAQNQQT | 3333 | 68.813 | 0.000 | 0.000 | 0.000 | 0.000 | 0.000 | 70.071 |
| KTESASGSPHSKAQNQQT | 3334 | 68.651 | 0.000 | 0.000 | 0.000 | 0.274 | 0.084 | 80.500 |
| KTNNGSGSPHSKAQNQQY | 3335 | 68.530 | 0.000 | 0.040 | 0.000 | 0.000 | 0.059 | 82.656 |
| KTSNGGGSPHSKAQNLQT | 3551 | 68.311 | 0.000 | 0.052 | 0.000 | 0.000 | 0.000 | 124.871 |
| KTDKMSGSPHSKAQNQQT | 3336 | 68.167 | 0.000 | 0.000 | 0.000 | 0.017 | 0.205 | 88.234 |
| KEVHGSGSPHSKAQNQQT | 3337 | 67.901 | 0.000 | 0.000 | 0.000 | 0.727 | 0.000 | 100.111 |
| KTINGSGSPHSKAQKLNT | 3338 | 67.782 | 0.073 | 0.092 | 0.000 | 1.232 | 0.201 | 68.637 |
| KTINGGGSPHSKSQNQHT | 3552 | 67.773 | 0.000 | 0.057 | 0.000 | 0.000 | 0.220 | 100.748 |
| KTVNGGGSPHSKAQSQQT | 3553 | 67.634 | 0.000 | 0.055 | 0.000 | 0.000 | 0.210 | 160.711 |
| KTTNGSGSPHSKAQYQHT | 3339 | 67.325 | 0.000 | 0.000 | 0.000 | 1.378 | 0.080 | 83.337 |
| KTISGSGSPHSKAQYQHT | 3340 | 66.739 | 0.000 | 0.000 | 0.000 | 0.000 | 0.191 | 59.822 |
| KTESTSGSPHSKAQNQQT | 3341 | 66.649 | 0.000 | 0.009 | 0.000 | 1.688 | 0.176 | 95.861 |
| KTINGSGSPHSKSQNVQT | 3342 | 66.627 | 0.000 | 0.190 | 0.000 | 0.202 | 0.188 | 56.672 |
| KSINGSGSPHSKAQAQQT | 3343 | 66.464 | 0.000 | 0.711 | 0.000 | 0.148 | 0.111 | 78.451 |
| KTVNGSGSPHSKAQNLQA | 3344 | 66.379 | 0.000 | 0.000 | 0.000 | 0.000 | 0.132 | 50.934 |
| KTVRDSGSPHSKAQNQQT | 3345 | 66.056 | 0.000 | 0.025 | 0.000 | 0.129 | 0.461 | 142.600 |
| KTFNASGSPHSKAPNQQT | 3346 | 65.392 | 0.208 | 0.000 | 0.000 | 0.215 | 0.156 | 66.275 |
| KTDRMSGSPHSKAQNQQT | 3347 | 65.143 | 0.000 | 0.000 | 0.000 | 0.332 | 0.103 | 104.890 |
| KTINGSGSPHSKAQTPPT | 3348 | 64.657 | 0.010 | 0.015 | 0.014 | 0.200 | 0.207 | 54.179 |
| ETIKGSGSPHSKAQNQQT | 3349 | 64.609 | 0.000 | 0.000 | 0.144 | 0.000 | 0.024 | 67.201 |
| KNHIGSGSPHSKAQNQQT | 3350 | 64.535 | 0.000 | 0.000 | 0.000 | 1.253 | 0.187 | 70.356 |
| KTINGSGSPHSKAQYQHA | 3351 | 64.435 | 0.000 | 0.000 | 0.024 | 0.993 | 0.097 | 57.278 |
| KTIPIDGSPHSKAQNQQT | 3554 | 64.421 | 0.000 | 0.047 | 0.000 | 0.234 | 0.936 | 76.826 |
| KTINGSGSPHSKAQGQQA | 3352 | 64.128 | 0.000 | 0.185 | 0.000 | 0.063 | 0.195 | 64.116 |
| KTFNGSGSPHNKAQNHQT | 3353 | 64.060 | 0.000 | 0.000 | 0.035 | 0.094 | 0.317 | 67.757 |
| KESDGSGSPHSKAQNQQT | 3354 | 63.766 | 0.000 | 0.000 | 0.000 | 0.567 | 0.146 | 115.231 |
| KTINGSGSPHSKAQPPAT | 3355 | 63.510 | 0.048 | 0.030 | 0.031 | 0.126 | 0.302 | 117.453 |
| KTINGSGSPHSKAQERPT | 3356 | 63.460 | 0.000 | 0.011 | 0.000 | 0.810 | 0.173 | 57.506 |
| KTIKGSGSPHSKAQDLQT | 3357 | 63.260 | 0.000 | 0.000 | 0.000 | 0.000 | 0.218 | 58.576 |
| KTDLKSGSPHSKAQNQQT | 3358 | 63.152 | 0.000 | 0.000 | 0.012 | 0.285 | 0.377 | 62.687 |
| KTINGGGSPHSKAQNPPT | 3555 | 63.041 | 0.000 | 0.082 | 0.000 | 0.000 | 0.057 | 64.045 |
| KTINGSGSPHSKAQAMHT | 3359 | 62.756 | 0.000 | 0.000 | 0.010 | 0.976 | 0.393 | 84.056 |
| KTVPNSGSPHSKAQNQQT | 3360 | 62.540 | 0.000 | 0.000 | 0.011 | 0.202 | 0.161 | 93.793 |
| KTVIGSGSPHSKALNQQT | 3361 | 62.358 | 0.000 | 0.310 | 0.000 | 0.062 | 0.245 | 60.369 |
| KTINGSGSPHSKAQHPST | 3362 | 62.255 | 0.000 | 0.044 | 0.000 | 1.345 | 0.301 | 101.103 |
| KTINGLGSPHSKSQNQQT | 3556 | 62.170 | 0.000 | 0.157 | 0.000 | 0.146 | 0.107 | 64.139 |
| KTINGTGSPHSKAQNQQM | 3557 | 62.151 | 0.000 | 0.000 | 0.000 | 0.000 | 0.000 | 62.376 |
| KTINGSGSPHSKAPGLQT | 3363 | 62.043 | 0.007 | 0.000 | 0.005 | 0.651 | 0.210 | 144.610 |
| KTINGSGSPHSKAQGIRT | 3364 | 61.952 | 0.041 | 0.000 | 0.012 | 0.897 | 0.502 | 155.013 |
| KTESHSGSPHSKAQNQQT | 3365 | 61.947 | 0.000 | 0.000 | 0.000 | 1.480 | 0.106 | 52.506 |
| KTINGSGSPHSKAQAPAT | 3366 | 61.934 | 0.000 | 0.169 | 0.015 | 0.696 | 0.197 | 127.420 |
| KTINGSGSPHSKSQSQQI | 3367 | 61.870 | 0.000 | 0.000 | 0.000 | 0.200 | 0.175 | 64.027 |
| KAEHGSGSPHSKAQNQQT | 3368 | 61.830 | 0.000 | 0.000 | 0.000 | 0.772 | 0.184 | 116.201 |
| KTEDRSGSPHSKAQNQQT | 3369 | 61.756 | 0.000 | 0.000 | 0.000 | 1.004 | 0.408 | 66.887 |
| KNCLGSGSPHSKAQNQQT | 3370 | 61.442 | 0.000 | 0.036 | 0.000 | 1.849 | 0.026 | 82.488 |
| KTDRGSGSPHSKAQNQQT | 3371 | 61.419 | 0.000 | 0.004 | 0.000 | 0.211 | 0.316 | 74.256 |
| KTINGSGSPHSKAQIPPT | 3372 | 61.258 | 0.000 | 0.000 | 0.000 | 0.758 | 0.115 | 87.661 |
| KTVKGSGSPHSKAQDQQT | 3373 | 61.175 | 0.000 | 0.041 | 0.000 | 0.432 | 0.090 | 58.114 |
| KNADGSGSPHSKAQNQQT | 3374 | 60.944 | 0.000 | 0.000 | 0.000 | 1.239 | 0.085 | 104.503 |
| KTDKVSGSPHSKAQNQQT | 3375 | 60.935 | 0.000 | 0.015 | 0.000 | 0.765 | 0.128 | 146.657 |
| KTITGSGSPHSKAQTQLT | 3376 | 60.846 | 0.160 | 8.992 | 0.000 | 0.000 | 0.000 | 55.640 |
| KTINGSGSPHSKAQAPST | 3377 | 60.696 | 0.200 | 0.005 | 0.000 | 0.751 | 0.263 | 115.528 |
| KNCVGSGSPHSKAQNQQT | 3378 | 60.535 | 0.000 | 0.000 | 0.000 | 0.018 | 0.282 | 96.175 |
| KTIRDAGSPHSKAQNQQT | 3558 | 60.346 | 0.000 | 0.000 | 0.000 | 0.141 | 0.251 | 113.179 |
| KTVKDSGSPHSKAQNQQT | 3379 | 60.216 | 0.000 | 0.019 | 0.000 | 0.443 | 0.251 | 87.334 |
| KNALGSGSPHSKAQNQQT | 3380 | 60.014 | 0.000 | 0.003 | 0.000 | 0.682 | 0.213 | 137.222 |
| KVINGSGSPHSKGQNQQT | 3381 | 60.001 | 0.000 | 0.000 | 0.031 | 0.264 | 0.157 | 68.532 |
| KTVNGGGSPHSKAQNQQS | 3559 | 59.871 | 0.062 | 0.020 | 0.000 | 0.080 | 0.185 | 61.847 |
| KTIQDGGSPHSKAQNQQT | 3560 | 59.865 | 0.000 | 0.000 | 0.116 | 1.435 | 0.789 | 87.522 |
| KTISGGGSPHSKAQNQQN | 3561 | 59.801 | 0.000 | 0.000 | 0.000 | 0.722 | 0.039 | 87.761 |
| KTSNASGSPHSKAHNQQT | 3382 | 59.607 | 0.000 | 0.078 | 0.067 | 0.031 | 0.050 | 67.967 |
| KTINGSGSPHSKAQNTYA | 3383 | 59.603 | 0.000 | 0.000 | 0.000 | 0.425 | 0.346 | 101.715 |
| KTINGSGSPHSKSQNQHI | 3384 | 59.438 | 0.000 | 0.099 | 0.000 | 0.111 | 0.108 | 76.025 |
| KTINGGGSPHSKAQDKQT | 3562 | 59.322 | 0.000 | 0.000 | 0.000 | 0.000 | 0.093 | 50.764 |
| KTEFVSGSPHSKAQNQQT | 3385 | 59.306 | 0.000 | 0.000 | 0.000 | 0.196 | 0.276 | 69.788 |
| KTVNGSGSPHSKAQNHLT | 3386 | 59.239 | 0.133 | 0.034 | 0.000 | 0.000 | 0.156 | 70.786 |
| KTREISGSPHSKAQNQQT | 3387 | 59.027 | 0.000 | 0.042 | 0.224 | 0.356 | 0.269 | 51.696 |
| KTINGSGSPHSKAQIGMT | 3388 | 59.013 | 0.081 | 106.528 | 0.000 | 1.003 | 0.248 | 134.585 |
| KTIDGSGSPHSKALNKQT | 3389 | 58.992 | 0.000 | 0.26 | 0.000 | 0.000 | 0.056 | 74.626 |
| KTIIGGGSPHSKAQNPQT | 3563 | 58.924 | 0.000 | 0.202 | 0.000 | 0.000 | 0.126 | 53.992 |
| KQGEGSGSPHSKAQNQQT | 3390 | 58.752 | 0.000 | 0.000 | 0.000 | 0.000 | 0.151 | 135.300 |
| KTINGTGSPHSKAPNQLT | 3564 | 58.738 | 0.000 | 0.000 | 0.000 | 0.229 | 0.035 | 86.939 |
| KTVNGSGSPHSKAQLQQT | 3391 | 58.681 | 0.315 | 0.465 | 0.045 | 0.529 | 0.333 | 81.201 |
| KTFNGGGSPHSKAQYQQT | 3565 | 58.609 | 0.000 | 0.000 | 0.000 | 0.163 | 0.045 | 72.618 |
| KSINGSGSPHSKTQSQQT | 3392 | 58.608 | 0.000 | 3.017 | 0.000 | 0.155 | 0.017 | 71.397 |
| KTVNGGGSPHSKAQHQQT | 3566 | 58.602 | 0.729 | 0.000 | 0.000 | 0.000 | 0.043 | 138.544 |
| KSEKGSGSPHSKAQNQQT | 3393 | 58.566 | 0.000 | 0.010 | 0.011 | 1.601 | 0.059 | 158.931 |
| KNVNGSGSPHSKAQNQQT | 3394 | 58.481 | 0.000 | 0.000 | 0.000 | 0.917 | 0.166 | 53.379 |
| KGGEGSGSPHSKAQNQQT | 3395 | 58.472 | 0.000 | 0.034 | 0.000 | 0.037 | 0.066 | 91.023 |
| KTINGSGSPHSKAQRMST | 3396 | 58.435 | 0.192 | 0.037 | 0.000 | 1.707 | 0.882 | 53.414 |
| KTINGSGSPHSKAQGILT | 3397 | 58.418 | 0.000 | 0.005 | 0.010 | 0.569 | 0.192 | 102.631 |
| KEFVGSGSPHSKAQNQQT | 3398 | 58.374 | 0.000 | 0.046 | 0.000 | 0.088 | 0.326 | 128.675 |
| KTIIGSGSPHSKAQDRQT | 3399 | 58.258 | 1.393 | 0.230 | 0.219 | 0.000 | 0.045 | 53.981 |
| KSDKGSGSPHSKAQNQQT | 3400 | 58.248 | 0.000 | 0.000 | 0.000 | 0.076 | 0.166 | 146.566 |
| KTEQVSGSPHSKAQNQQT | 3401 | 58.247 | 0.000 | 0.000 | 0.000 | 0.000 | 0.081 | 88.487 |
| KTEHVSGSPHSKAQNQQT | 3402 | 58.228 | 0.000 | 0.024 | 0.000 | 0.433 | 0.141 | 71.410 |
| KTINGSGSPHSKARDWQT | 3403 | 58.216 | 0.000 | 0.005 | 0.000 | 0.800 | 0.259 | 120.704 |
| KTENASGSPHSKAQNQQT | 3404 | 58.187 | 0.000 | 0.038 | 0.000 | 0.371 | 0.129 | 88.439 |
| KEVQGSGSPHSKAQNQQT | 3405 | 58.125 | 0.000 | 0.000 | 0.000 | 0.657 | 0.000 | 168.220 |
| KTINGSGSPHSKAQNTHD | 3406 | 58.108 | 0.000 | 0.027 | 0.000 | 0.410 | 0.126 | 81.189 |
| KTINGSGSPHSKAPNLQI | 3407 | 58.022 | 0.000 | 0.044 | 0.000 | 1.548 | 0.243 | 55.714 |
| KTINGSGSPHSKAQERST | 3408 | 58.021 | 0.000 | 0.011 | 0.005 | 0.829 | 0.409 | 87.656 |
| KTSNGSGSPHSKAQNYQT | 3409 | 57.894 | 0.000 | 0.082 | 0.000 | 0.000 | 0.110 | 63.681 |
| KTEYISGSPHSKAQNQQT | 3410 | 57.891 | 0.000 | 0.000 | 0.000 | 0.076 | 0.075 | 57.620 |
| KTINGSGSPHSKAQRTCT | 3411 | 57.863 | 0.000 | 0.140 | 0.129 | 1.855 | 1.716 | 90.146 |
| KTINGSGSPHSKAQIGHT | 3412 | 57.769 | 0.024 | 0.000 | 0.000 | 0.281 | 0.154 | 99.262 |
| KNCWGSGSPHSKAQNQQT | 3413 | 57.756 | 0.000 | 0.000 | 0.000 | 0.000 | 0.092 | 59.888 |
| KTINGSGSPHSKAQGAIT | 3414 | 57.627 | 0.000 | 0.000 | 0.000 | 0.594 | 0.161 | 95.696 |
| KTDVNSGSPHSKAQNQQT | 3415 | 57.593 | 0.000 | 0.000 | 0.000 | 0.000 | 0.331 | 66.127 |
| KSDIGSGSPHSKAQNQQT | 3416 | 57.592 | 0.000 | 0.000 | 0.000 | 0.844 | 0.128 | 107.342 |
| KTINGSGSPHSKAQVPPT | 3417 | 57.316 | 0.000 | 0.012 | 0.000 | 0.257 | 0.200 | 90.220 |
| KTINGSGSPHSKAQVQQI | 3418 | 57.308 | 0.000 | 1.113 | 0.000 | 0.000 | 0.113 | 61.957 |
| KTINGSGSPHSKALMRQT | 3419 | 57.234 | 0.060 | 0.036 | 0.100 | 1.798 | 0.517 | 81.332 |
| KTINGSGSPHSKAQYSVT | 3420 | 57.130 | 0.000 | 0.093 | 0.000 | 1.235 | 0.302 | 60.023 |
| KNSIGSGSPHSKAQNQQT | 3421 | 57.101 | 0.000 | 0.052 | 0.000 | 0.083 | 0.074 | 97.381 |
| KTINGSGSPHSKVPNLQT | 3422 | 57.046 | 0.000 | 0.029 | 0.000 | 0.459 | 0.082 | 50.474 |
| KAINGSGSPHSKAQSQQI | 3423 | 56.976 | 0.000 | 0.000 | 0.000 | 0.000 | 0.000 | 57.052 |
| KTINGSGSPHSKAQAITT | 3424 | 56.924 | 0.000 | 0.000 | 0.000 | 1.239 | 0.438 | 75.250 |
| KTINGSGSPHSKAQKTLT | 3425 | 56.844 | 0.000 | 0.017 | 0.009 | 1.800 | 1.400 | 66.415 |
| KTVNGSGSPHSKAQNQWT | 3426 | 56.823 | 0.000 | 0.000 | 0.299 | 0.000 | 0.219 | 69.906 |
| KTINGSGSPHSKAQLHHT | 3427 | 56.815 | 0.025 | 0.000 | 0.010 | 0.712 | 0.368 | 58.418 |
| KTEQTSGSPHSKAQNQQT | 3428 | 56.683 | 0.000 | 0.045 | 0.000 | 0.792 | 0.430 | 59.360 |
| KTINGSGSPHSKAQNIII | 3429 | 56.630 | 0.000 | 0.062 | 0.123 | 0.099 | 0.056 | 76.742 |
| KNSLGSGSPHSKAQNQQT | 3430 | 56.621 | 0.000 | 0.028 | 0.000 | 0.308 | 0.162 | 101.942 |
| KTIPMEGSPHSKAQNQQT | 3567 | 56.560 | 0.000 | 0.000 | 0.000 | 1.824 | 0.371 | 89.951 |
| KTINGSGSPHSKAQGHHT | 3431 | 56.559 | 0.000 | 0.000 | 0.000 | 0.632 | 0.117 | 71.050 |
| KTDRTSGSPHSKAQNQQT | 3432 | 56.466 | 0.000 | 0.000 | 0.000 | 0.062 | 0.160 | 148.498 |
| KTINGSGSPHSKAQSKVT | 3433 | 56.373 | 0.000 | 0.050 | 0.014 | 1.021 | 0.390 | 76.115 |
| KEVVGSGSPHSKAQNQQT | 3434 | 56.371 | 0.000 | 0.000 | 0.000 | 0.000 | 0.323 | 116.964 |
| KTINGSGSPHSKAQLPST | 3435 | 56.238 | 0.005 | 4.258 | 0.001 | 1.040 | 0.185 | 84.918 |
| KTINGSGSPHSKAIGKQT | 3436 | 56.158 | 0.000 | 0.000 | 0.000 | 0.887 | 0.088 | 110.132 |
| KTEPTSGSPHSKAQNQQT | 3437 | 56.134 | 0.000 | 0.000 | 0.000 | 0.06 | 0.527 | 143.397 |
| KTVNGGGSPHSKSQNQQT | 3568 | 56.114 | 0.116 | 0.000 | 0.000 | 0.000 | 0.040 | 170.548 |
| KTINGSGSPHSKAQAIHT | 3438 | 56.047 | 0.000 | 0.000 | 212.32 | 0.887 | 0.890 | 81.908 |
| KTINGSGSPHSKAQHGLT | 3439 | 55.999 | 0.000 | 0.000 | 0.101 | 1.913 | 0.244 | 117.191 |
| KSELGSGSPHSKAQNQQT | 3440 | 55.997 | 0.000 | 0.005 | 0.000 | 0.881 | 0.239 | 120.521 |
| KTINGSGSPHSKAQFMCT | 3441 | 55.916 | 0.000 | 0.000 | 0.000 | 0.078 | 0.448 | 81.959 |
| KTINVSGSPHSKAQGQQT | 3442 | 55.870 | 0.000 | 0.191 | 0.000 | 0.592 | 0.040 | 87.211 |
| KTINGGGSPHSKAQNQMT | 3569 | 55.778 | 0.000 | 0.000 | 0.000 | 0.866 | 0.012 | 73.177 |
| KTVNGSGSPHSKAQHLQT | 3443 | 55.739 | 0.091 | 0.036 | 0.000 | 0.062 | 0.409 | 62.743 |
| KTIRENGSPHSKAQNQQT | 3570 | 55.605 | 0.000 | 0.000 | 0.016 | 0.131 | 0.257 | 95.931 |
| KTINGSGSPHSKTQNHQN | 3444 | 55.551 | 0.000 | 0.048 | 0.000 | 0.000 | 0.099 | 64.846 |
| KTINGSGSPHSKAQPART | 3445 | 55.513 | 0.000 | 0.000 | 0.328 | 1.294 | 0.991 | 127.301 |
| KTVNGSGSPHSKAQSLQT | 3446 | 55.497 | 0.000 | 0.060 | 0.000 | 0.000 | 0.143 | 69.033 |
| KTINGSGSPHSKSQSQLT | 3447 | 55.430 | 0.000 | 0.035 | 0.000 | 0.050 | 0.013 | 125.577 |
| KTINGSASPHSKAHSQQT | 3571 | 55.293 | 0.000 | 0.000 | 0.000 | 0.000 | 0.166 | 66.252 |
| KTWQNSGSPHSKAQNQQT | 3448 | 55.245 | 0.000 | 0.000 | 0.000 | 0.111 | 0.265 | 114.258 |
| KTINGSGSPHSKAQDRQS | 3449 | 55.137 | 1.146 | 0.016 | 0.106 | 0.644 | 0.086 | 55.701 |
| KTINGSGSPHSKAQMPST | 3450 | 54.986 | 1.691 | 0.039 | 0.028 | 0.450 | 0.202 | 114.331 |
| KTNNGGGSPHSKAQNLQT | 3572 | 54.963 | 0.000 | 0.000 | 0.000 | 0.000 | 0.089 | 80.506 |
| KTINGSGSPHSKAQGSLT | 3451 | 54.717 | 0.000 | 0.006 | 0.013 | 0.480 | 0.298 | 142.786 |
| KTEVTSGSPHSKAQNQQT | 3452 | 54.663 | 0.000 | 0.000 | 0.000 | 0.323 | 0.185 | 81.482 |
| KSINGGGSPHSKAQYQQT | 3573 | 54.612 | 0.000 | 0.000 | 0.000 | 0.105 | 0.010 | 65.952 |
| KTVIGSGSPHSKSQNQQT | 3453 | 54.603 | 0.000 | 0.000 | 0.000 | 0.000 | 0.106 | 69.121 |
| KAVNVSGSPHSKAQNQQT | 3454 | 54.586 | 0.000 | 0.000 | 0.000 | 0.000 | 0.023 | 57.835 |
| KTVNGNGSPHSKSQNQQT | 3574 | 54.586 | 0.000 | 0.000 | 0.000 | 0.256 | 0.168 | 95.384 |
| KTDRNSGSPHSKAQNQQT | 3455 | 54.495 | 0.000 | 0.000 | 0.000 | 0.823 | 0.241 | 85.823 |
| KTINGSGSPHSKAQVPAT | 3456 | 54.475 | 0.000 | 0.002 | 0.000 | 0.782 | 0.223 | 137.743 |
| KGVLGSGSPHSKAQNQQT | 3457 | 54.472 | 0.000 | 0.007 | 0.027 | 0.359 | 0.189 | 145.740 |
| KTLNGNGSPHSKAQNLQT | 3575 | 54.458 | 0.668 | 0.000 | 0.000 | 0.161 | 0.172 | 159.134 |
| KAINGSGSPHSKAQDKQT | 3458 | 54.452 | 0.000 | 0.000 | 0.057 | 0.044 | 0.223 | 56.004 |
| KTSNGSGSPHSKAHYQQT | 3459 | 54.414 | 0.000 | 0.251 | 0.000 | 0.249 | 0.204 | 54.162 |
| KTINGSGSPHSKAQVPST | 3460 | 54.366 | 0.000 | 1.001 | 0.000 | 0.202 | 0.139 | 117.223 |
| KTINGSGSSHSKAQNQQT | 3576 | 54.292 | 1.709 | 1.870 | 1.287 | 1.075 | 0.458 | 67.731 |
| KTELRSGSPHSKAQNQQT | 3461 | 54.289 | 0.000 | 0.007 | 0.040 | 0.790 | 0.239 | 57.814 |
| KNINGSGSPHSKAQNHQT | 3462 | 54.248 | 0.000 | 0.034 | 0.000 | 0.340 | 0.075 | 74.979 |
| KTVNGGGSPHSKAQNHQT | 3577 | 54.246 | 0.375 | 0.024 | 0.000 | 0.000 | 0.146 | 67.188 |
| KTINGSGSPHSKARGEQT | 3463 | 54.207 | 0.025 | 0.006 | 0.005 | 0.309 | 0.327 | 128.098 |
| KTINGGGSPHSKAQYQHT | 3578 | 54.188 | 0.000 | 0.000 | 0.000 | 0.000 | 0.223 | 82.256 |
| KTEDLSGSPHSKAQNQQT | 3464 | 54.156 | 0.000 | 0.000 | 0.000 | 1.193 | 0.132 | 70.198 |
| KTINGSGSPHSKAPGQQT | 3465 | 54.071 | 0.065 | 0.000 | 0.004 | 0.542 | 0.179 | 73.440 |
| KTIPKNGSPHSKAQNQQT | 3579 | 53.824 | 0.000 | 0.032 | 0.000 | 0.115 | 0.178 | 77.458 |
| KTINGSGSPHSKAQSLQI | 3466 | 53.778 | 0.000 | 0.186 | 0.000 | 0.022 | 0.047 | 51.543 |
| KTINGSGSPHSKRLEQQT | 3467 | 53.512 | 0.000 | 0.118 | 0.003 | 0.161 | 0.292 | 71.704 |
| KTERGSGSPHSKAQNQQT | 3468 | 53.475 | 0.000 | 0.030 | 0.000 | 1.416 | 0.175 | 85.368 |
| KTVNGSGSPHSKAPNQQT | 3469 | 53.444 | 0.833 | 2.206 | 0.006 | 0.156 | 0.178 | 58.080 |
| KTSNGSGSPHSKAQNQST | 3470 | 53.353 | 0.000 | 0.000 | 0.000 | 0.000 | 0.014 | 120.897 |
| KTINGSGSPHSKAQKVIT | 3471 | 53.273 | 0.000 | 0.000 | 0.000 | 0.357 | 0.402 | 95.147 |
| KTEGISGSPHSKAQNQQT | 3472 | 53.270 | 0.000 | 0.000 | 0.000 | 0.000 | 0.010 | 78.303 |
| KTINGSGSPHSKAQNNDQ | 3473 | 53.226 | 0.000 | 0.000 | 0.000 | 0.593 | 0.046 | 59.664 |
| KTINGSGSPHSKAQSVHT | 3474 | 53.226 | 0.000 | 0.004 | 0.000 | 0.446 | 0.217 | 76.110 |
| KTINGSGSPHSKAQPLGT | 3475 | 53.049 | 0.015 | 0.004 | 0.001 | 0.515 | 0.222 | 68.656 |
| KTINKEGSPHSKAQNQQT | 3580 | 53.006 | 0.000 | 0.029 | 0.000 | 0.177 | 0.111 | 64.520 |
| KTCNASGSPHSKAQNQQT | 3476 | 52.998 | 0.000 | 0.011 | 0.000 | 0.897 | 0.141 | 67.934 |
| KAINGSGSPHSKAHNQET | 3477 | 52.973 | 0.000 | 0.030 | 0.000 | 0.035 | 0.058 | 71.809 |
| KTEGLSGSPHSKAQNQQT | 3478 | 52.891 | 0.000 | 0.000 | 0.020 | 0.104 | 0.155 | 104.529 |
| KTRDASGSPHSKAQNQQT | 3479 | 52.861 | 0.000 | 0.000 | 0.010 | 1.062 | 0.402 | 52.089 |
| KTSNGSGSPHSKAQNLQI | 3480 | 52.843 | 0.000 | 0.000 | 1.605 | 0.178 | 0.214 | 74.823 |
| KTGNGSGSPHSKAQIQQT | 3481 | 52.809 | 0.000 | 0.000 | 0.000 | 0.000 | 0.012 | 98.291 |
| KTVNGGGSPHSKAQNLQT | 3581 | 52.788 | 0.000 | 0.031 | 0.000 | 0.000 | 0.165 | 83.215 |
| KTDRSSGSPHSKAQNQQT | 3482 | 52.737 | 0.000 | 0.000 | 0.000 | 0.995 | 0.085 | 123.421 |
| KTINGSGSPHSKAQVRNT | 3483 | 52.735 | 0.000 | 0.101 | 0.011 | 0.230 | 0.423 | 68.893 |
| KTINGSGSPHSKAPSNQT | 3484 | 52.680 | 1.494 | 4.762 | 0.003 | 0.330 | 0.208 | 87.951 |
| KTINGSGSPHSKAQVGHT | 3485 | 52.624 | 0.000 | 0.000 | 0.006 | 0.535 | 0.192 | 106.448 |
| KNAIGSGSPHSKAQNQQT | 3486 | 52.516 | 0.000 | 0.000 | 0.000 | 0.165 | 0.198 | 117.939 |
| KAENGSGSPHSKAQNQQT | 3487 | 52.487 | 0.000 | 0.157 | 0.029 | 0.000 | 0.242 | 120.256 |
| KTINGSGSPHSKAQRDIT | 3488 | 52.415 | 0.098 | 0.000 | 0.008 | 1.784 | 0.605 | 88.122 |
| KTINGSGSPHSKAQMPNT | 3489 | 52.408 | 0.084 | 0.036 | 0.025 | 0.057 | 0.359 | 66.040 |
| KTVNGSGSPHSKSQNQQT | 3490 | 52.395 | 0.033 | 0.077 | 0.013 | 0.105 | 0.175 | 58.000 |
| KTIPAIGSPHSKAQNQQT | 3582 | 52.346 | 0.000 | 0.009 | 0.000 | 0.034 | 0.134 | 51.949 |
| KTINGSGSPHSKARGLQT | 3491 | 52.275 | 0.000 | 0.000 | 0.036 | 1.235 | 1.425 | 169.881 |
| KTELGSGSPHSKAQNQQT | 3492 | 52.232 | 0.000 | 0.007 | 0.006 | 0.532 | 0.088 | 87.314 |
| KAETGSGSPHSKAQNQQT | 3493 | 52.219 | 0.000 | 0.047 | 0.581 | 0.009 | 0.188 | 132.940 |
| KTINGSGSPHSKLQKQQT | 3494 | 52.144 | 0.615 | 0.477 | 1.071 | 1.113 | 0.429 | 61.833 |
| KTINGSGSPHSKAPSLQT | 3495 | 52.137 | 0.041 | 1.614 | 0.002 | 0.902 | 0.222 | 70.363 |
| KTINGSGSPHSKAQRDQT | 3496 | 51.897 | 0.069 | 0.014 | 0.040 | 0.867 | 0.554 | 102.317 |
| KTDVGSGSPHSKAQNQQT | 3497 | 51.849 | 0.000 | 0.007 | 0.000 | 0.385 | 0.560 | 115.774 |
| KTINGSGSPHSKNRDQQT | 3498 | 51.830 | 0.000 | 0.008 | 0.000 | 0.480 | 0.138 | 100.300 |
| KSINGSGSPHSKAPNLQT | 3499 | 51.812 | 0.000 | 0.256 | 0.000 | 0.085 | 0.139 | 59.270 |
| KTINGSGSPHSKAQAKGT | 3500 | 51.727 | 0.048 | 0.016 | 0.000 | 0.271 | 0.525 | 104.917 |
| KTVNGSGSPHSKAQDKQT | 3501 | 51.580 | 0.428 | 0.000 | 0.069 | 0.041 | 0.063 | 69.225 |
| KTINGGGSPHSKAQNPQA | 3583 | 51.574 | 0.000 | 0.000 | 0.000 | 0.192 | 0.000 | 102.792 |
| KTINGSGSPHSKAQSAHT | 3502 | 51.569 | 0.068 | 0.070 | 0.000 | 0.589 | 0.249 | 79.498 |
| KTINGNGSPHSKSQNQHT | 3584 | 51.379 | 0.000 | 0.054 | 0.000 | 0.000 | 0.082 | 56.614 |
| KTVPTSGSPHSKAQNQQT | 3503 | 51.348 | 0.013 | 0.000 | 0.000 | 1.017 | 0.338 | 102.651 |
| KTIDGSGSPHSKSQNHQT | 3504 | 51.307 | 0.000 | 0.000 | 0.000 | 0.000 | 0.269 | 63.174 |
| KTDVKSGSPHSKAQNQQT | 3505 | 51.296 | 0.000 | 0.000 | 0.000 | 0.515 | 0.224 | 53.601 |
| KAINRSGSPHSKAQDQQT | 3506 | 51.262 | 0.000 | 0.000 | 0.000 | 0.000 | 0.036 | 54.631 |
| KTINGSGSPHSKAQSTMT | 3507 | 51.249 | 0.018 | 0.002 | 0.002 | 0.321 | 0.341 | 73.213 |
| KTVNASGSPHSKAQNQLT | 3508 | 51.249 | 0.000 | 0.000 | 0.000 | 0.000 | 0.268 | 99.559 |
| KTINGSGSPHSKAQREMT | 3509 | 51.076 | 0.000 | 24.900 | 143.49 | 1.564 | 0.476 | 70.961 |
| KTVHGSGSPHSKAQSQQT | 3510 | 51.057 | 0.000 | 0.000 | 0.000 | 0.143 | 0.146 | 54.185 |
| KTINGGGSPHSKSQNRQT | 3585 | 51.017 | 0.000 | 0.000 | 0.000 | 0.000 | 0.421 | 149.370 |
| KTINGSGSPHSKAQYRAT | 3511 | 51.008 | 0.000 | 0.158 | 0.000 | 0.690 | 0.120 | 50.650 |
| KTINGGGSPHSKAQRQQT | 3586 | 50.998 | 0.000 | 0.041 | 0.000 | 0.991 | 0.142 | 147.942 |
| KTEPMSGSPHSKAQNQQT | 3512 | 50.960 | 0.203 | 0.000 | 0.000 | 1.816 | 0.415 | 126.322 |
| KTINGSGSPHSKNQWQQT | 3513 | 50.800 | 0.000 | 0.044 | 0.047 | 0.111 | 0.324 | 65.506 |
| KETAGSGSPHSKAQNQQT | 3514 | 50.762 | 0.000 | 0.027 | 0.000 | 1.706 | 0.054 | 212.795 |
| KTINGSGSPHSKAQRMNT | 3515 | 50.686 | 0.000 | 108.747 | 0.019 | 0.943 | 0.264 | 97.975 |
| KNNLGSGSPHSKAQNQQT | 3516 | 50.670 | 0.000 | 0.019 | 0.000 | 0.406 | 0.121 | 102.408 |
| KTINGSGSPHAKAQNHQT | 3517 | 50.667 | 0.211 | 0.140 | 0.051 | 0.101 | 0.090 | 80.603 |
| KTIIKNGSPHSKAQNQQT | 3587 | 50.587 | 0.000 | 0.000 | 0.000 | 0.000 | 0.751 | 75.547 |
| KTINGSGSPHSYHVNQQT | 3588 | 50.486 | 0.000 | 0.056 | 0.059 | 0.528 | 0.275 | 179.489 |
| KTINGSGSPHSKAGDSQT | 3518 | 50.457 | 0.614 | 0.236 | 0.008 | 1.062 | 0.071 | 74.355 |
| KTINGSGSPHSKLKSQQT | 3519 | 50.368 | 0.000 | 0.296 | 0.000 | 1.796 | 1.096 | 95.240 |
| KTINGSGSPHSKAQKIST | 3520 | 50.285 | 0.000 | 0.000 | 0.088 | 0.108 | 0.302 | 51.115 |
| KTEYNSGSPHSKAQNQQT | 3521 | 50.256 | 0.000 | 0.000 | 0.000 | 0.000 | 0.009 | 62.679 |
| KTINGSGSPHSKAPSMQT | 3522 | 50.249 | 0.000 | 0.000 | 0.004 | 0.941 | 0.460 | 75.504 |
| EAINGSGSPHSKAQNQQT | 3523 | 50.243 | 0.629 | 0.094 | 0.000 | 0.057 | 1.519 | 117.305 |
| KTINGSGSPHSKASPRQT | 3524 | 50.227 | 0.088 | 0.005 | 0.068 | 1.761 | 0.530 | 67.241 |
| KTINGSGSPHSKRMEQQT | 3525 | 50.177 | 0.000 | 0.000 | 0.000 | 1.327 | 0.208 | 81.769 |
| KTINGSGSPHSKAQYQNT | 3526 | 50.099 | 0.000 | 0.008 | 0.000 | 0.017 | 0.119 | 71.846 |
| KTERVSGSPHSKAQNQQT | 3589 | 96.943 | 0.000 | 0.000 | 0.000 | 0.000 | 0.144 | 135.438 |
| KAEIGHDSPHKSGQNQQT | 1754 | 63.249 | 0.000 | 0.000 | 0.000 | 0.060 | 0.024 | 27.173 |

Table 16 provides the peptide sequence of 341 matured capsid variants, and the fold enrichment of these matured capsid variants relative to the AAV9 control that demonstrated a 75-fold or greater increase in expression in the brain of NHPs relative to the AAV9 control and had a fold-change in expression that was less than 2 relative to the AAV9 control in the liver and the DRG.

TABLE 16
NGS fold-enrichment of TTM-001 and TTM-002 matured
AAV capsid variants in the brain of NHPs

| | | Fold Enrichment relative to AAV9 | | | | | | |
|---|---|---|---|---|---|---|---|---|
| | SEQ | | | | | Liver | Liver | |
| | ID | Brain | DRG | Heart | Muscle | RNA | DNA | Brain |
| Sequence | NO: | (NHP) | (NHP) | (NHP) | (NHP) | (NHP) | (NHP) | (Mouse) |
| KTFNRSGSPHSKAQNQQI | 3591 | 86.359 | 0.000 | 113.67 | 0.000 | 0.000 | 0.092 | 25.568 |
| KTIIGSGSPHSKAQNRHT | 3239 | 217.176 | 0.000 | 0.000 | 0.000 | 0.000 | 0.000 | 210.515 |
| KTFPGSGSPHSKVQNQQT | 3240 | 199.720 | 0.000 | 0.000 | 0.000 | 0.000 | 0.967 | 97.703 |
| KTEKMSGSPHSKAQNQQT | 3241 | 169.461 | 0.523 | 0.000 | 0.000 | 0.000 | 0.158 | 109.161 |
| KAINGHDSPHKSGQIRQT | 3606 | 108.510 | 0.000 | 23.908 | 0.000 | 0.132 | 0.261 | 8.862 |
| KTINGHDSPHKIGQNQHA | 3607 | 77.321 | 0.000 | 18.836 | 0.028 | 0.220 | 0.132 | 7.578 |
| KEINGRGSPHSKAQNQQT | 3527 | 134.390 | 0.239 | 0.000 | 0.000 | 0.000 | 0.232 | 52.311 |
| KTVNRNGSPHSKAQNQQT | 3528 | 133.016 | 0.000 | 0.416 | 0.000 | 0.000 | 0.000 | 85.361 |
| KAINGYDSPHKSGQKQQT | 3608 | 83.803 | 0.041 | 9.491 | 0.000 | 0.031 | 0.150 | 13.057 |
| KTVNGSGSPHSKARDQQT | 3242 | 124.789 | 0.123 | 0.039 | 0.312 | 0.569 | 0.454 | 132.137 |
| KTESGHDSPHKSGQNQQT | 3609 | 86.513 | 0.000 | 7.414 | 0.000 | 0.000 | 0.038 | 13.163 |
| KTINGHDSPHKSGQSVQT | 3610 | 75.748 | 0.010 | 6.808 | 0.000 | 0.165 | 0.058 | 9.321 |
| KTFNGSGSPHSKAPNLQT | 3243 | 121.436 | 0.000 | 0.167 | 0.000 | 0.000 | 0.015 | 168.920 |
| KTEKTSGSPHSKAQNQQT | 3244 | 120.337 | 0.000 | 0.355 | 0.000 | 0.000 | 0.119 | 101.467 |
| TTINGHDSPHKSGQNQQT | 3611 | 108.963 | 1.512 | 3.445 | 0.869 | 0.659 | 1.109 | 14.788 |
| KTINGHESPHKSGRSQQT | 3612 | 97.106 | 0.000 | 3.329 | 0.022 | 0.000 | 0.181 | 9.378 |
| KTINGSGSPHSKAHVRQT | 3245 | 119.798 | 0.000 | 0.000 | 0.262 | 0.694 | 1.039 | 165.590 |
| KTVNGSGSPHSKAPNQHT | 3246 | 117.207 | 0.000 | 0.109 | 0.000 | 0.000 | 0.074 | 51.008 |
| KTEKISGSPHSKAQNQQT | 3247 | 116.603 | 0.000 | 0.000 | 0.000 | 0.000 | 0.426 | 102.978 |
| KTINGPGSPHSKAHNQQT | 3529 | 115.742 | 0.146 | 0.000 | 0.235 | 0.000 | 0.513 | 52.508 |
| KTINGHDSPHKSGQNKLE | 3613 | 76.204 | 0.000 | 1.430 | 0.000 | 0.015 | 0.031 | 12.419 |
| KTVNGSGSPHSKTQSQQT | 3248 | 115.086 | 0.000 | 0.726 | 0.000 | 0.000 | 0.340 | 63.248 |
| TTINGSGSPHSKAQNQQT | 3249 | 114.856 | 1.340 | 14.856 | 0.827 | 1.281 | 0.957 | 72.058 |
| KSINESGSPHSKAQNQQI | 3250 | 113.833 | 0.000 | 0.000 | 0.000 | 0.000 | 0.000 | 67.649 |
| KTINGHDSPHKTGQNQQK | 3614 | 77.562 | 0.000 | 1.056 | 0.000 | 0.000 | 0.000 | 6.379 |
| KTERTSGSPHSKAQNQQT | 3251 | 112.957 | 0.000 | 0.009 | 0.000 | 1.128 | 0.207 | 117.374 |
| KTINGSGSPHSKAQPAKT | 3252 | 111.472 | 0.331 | 0.000 | 1.089 | 0.044 | 1.796 | 215.275 |
| KTINGRGSPHKRGQNQQT | 3837 | 120.889 | 0.100 | 0.814 | 0.434 | 0.458 | 0.614 | 13.988 |
| KTINGSGSPHTKAQNPPT | 3592 | 147.061 | 0.000 | 0.727 | 0.000 | 0.000 | 0.000 | 34.425 |
| KTEKSSGSPHSKAQNQQT | 3253 | 107.470 | 0.000 | 0.016 | 0.014 | 0.977 | 0.179 | 100.177 |
| KAINGHDNPHKSGQNQQT | 3615 | 88.906 | 0.297 | 0.721 | 0.482 | 0.222 | 0.130 | 9.702 |
| KTSYGNGSPHSKAQNQQT | 3530 | 105.937 | 0.000 | 0.000 | 0.000 | 0.000 | 0.114 | 105.894 |
| KTINGQDSPHKSGQHQQA | 3616 | 85.657 | 1.127 | 0.579 | 0.000 | 0.193 | 0.557 | 5.582 |
| KTEKGSGSPHSKAQNQQT | 3254 | 105.614 | 0.053 | 0.031 | 0.000 | 0.586 | 0.169 | 84.653 |
| KTINGSGSPHSKSQTQQN | 3255 | 104.474 | 0.000 | 0.131 | 0.000 | 0.084 | 0.038 | 54.021 |
| KTERISGSPHSKAQNQQT | 3256 | 103.692 | 0.000 | 0.000 | 0.000 | 0.062 | 0.370 | 89.637 |
| KTERASGSPHSKAQNQQT | 3257 | 103.669 | 0.000 | 0.000 | 0.000 | 0.127 | 0.070 | 115.550 |
| KSINGHDSPHKSGQIQHT | 3617 | 87.598 | 0.000 | 0.480 | 0.000 | 0.714 | 0.347 | 13.872 |
| KELHGSGSPHSKAQNQQT | 3258 | 102.680 | 0.000 | 0.000 | 0.000 | 1.634 | 0.592 | 96.554 |
| KAINGSGSPHSKAQNLAT | 3259 | 101.954 | 0.000 | 10.954 | 8.655 | 0.298 | 0.239 | 116.685 |
| KTVNGSGSPHSKSQNQLT | 3260 | 101.327 | 0.000 | 0.035 | 0.000 | 0.000 | 0.025 | 80.716 |
| KAINGHDSPHKSGPRQQT | 3618 | 145.142 | 0.000 | 0.408 | 0.000 | 0.000 | 0.000 | 8.259 |
| KTVNGHDSPHKSGHTQQT | 3619 | 82.246 | 0.000 | 0.378 | 1.142 | 0.000 | 0.123 | 6.160 |
| KSINGHDSPHKSGQRQHT | 3620 | 80.132 | 0.000 | 0.357 | 0.000 | 0.000 | 0.000 | 9.851 |
| KTERNSGSPHSKAQNQQT | 3261 | 99.892 | 0.000 | 0.000 | 0.000 | 0.000 | 0.107 | 87.392 |
| KSLNGSGSPHTKAQNQQT | 3593 | 81.515 | 0.197 | 0.333 | 0.000 | 0.000 | 0.085 | 45.140 |
| KSVNGNGSPHSKAQNQQT | 3531 | 99.385 | 0.000 | 1.329 | 0.000 | 0.359 | 0.079 | 51.016 |
| KAINGHDSPHKSAQSQQT | 3621 | 95.204 | 0.146 | 0.310 | 0.000 | 0.699 | 0.058 | 14.595 |
| KSIYGHESPHKSGQNQQS | 3622 | 90.947 | 0.817 | 0.310 | 0.000 | 0.000 | 0.243 | 8.064 |
| KTFNGSGSPHSKAQGQQT | 3262 | 99.253 | 0.000 | 0.208 | 0.000 | 0.128 | 0.099 | 81.459 |
| KTVNGHDSPHKSLQNQQT | 3623 | 112.925 | 0.000 | 0.301 | 0.059 | 0.000 | 0.322 | 16.726 |
| KTINGSGSPHGWVQNQQT | 3532 | 97.122 | 0.000 | 0.000 | 0.000 | 1.240 | 1.975 | 290.720 |
| KTINGHGSPHSKAQNPQT | 3838 | 83.478 | 0.000 | 0.288 | 0.219 | 0.000 | 0.260 | 11.001 |
| KTSNGYDSPHKSGQKQQT | 3624 | 77.001 | 0.032 | 0.286 | 0.000 | 0.000 | 0.016 | 8.813 |
| KTVNGHDSPHKSGRNQET | 3625 | 102.695 | 0.000 | 0.286 | 0.000 | 0.000 | 0.027 | 11.958 |
| KTTNGHDSPHKSGQTQLT | 3626 | 115.637 | 0.000 | 0.283 | 0.000 | 0.052 | 0.321 | 17.885 |
| KAINGHDSPHKSEKNQQT | 3627 | 77.103 | 0.000 | 0.274 | 0.000 | 0.000 | 0.000 | 26.868 |
| KTERVSGSPHSKAQNQQT | 3263 | 96.943 | 0.000 | 0.000 | 0.000 | 0.000 | 0.144 | 135.438 |
| KTINGSGSPHSKALNRQS | 3264 | 96.843 | 0.136 | 0.532 | 0.000 | 0.042 | 0.178 | 55.945 |
| KTERLSGSPHSKAQNQQT | 3265 | 95.857 | 0.000 | 0.004 | 0.005 | 0.126 | 0.260 | 102.372 |
| KIINGRDSPHKSGQDQQT | 3628 | 78.773 | 0.000 | 0.254 | 0.000 | 0.000 | 0.156 | 16.132 |
| KTDNGSGSPHSKAHNQQT | 3266 | 95.164 | 0.000 | 0.000 | 0.000 | 0.000 | 0.027 | 55.313 |
| KTFHGSGSPHSKTQNQQT | 3267 | 94.714 | 0.000 | 0.210 | 0.120 | 0.000 | 0.000 | 51.119 |
| KTISGHDSPHKTGHNQQT | 3629 | 92.490 | 0.000 | 0.233 | 0.057 | 0.730 | 0.000 | 8.823 |
| KTVNAHDSPHKSGQNQLT | 3630 | 79.137 | 0.000 | 0.233 | 0.178 | 0.753 | 0.045 | 29.254 |
| KTINGGGSPHSKAQTQQI | 3533 | 92.345 | 0.000 | 0.000 | 0.000 | 0.000 | 0.023 | 54.199 |
| KSINGYDSPHKSGQTQQT | 3631 | 79.227 | 1.817 | 0.226 | 0.000 | 0.000 | 1.148 | 4.497 |
| KTINGHESPHKSGQTQQI | 3632 | 86.089 | 0.000 | 0.222 | 0.000 | 0.000 | 0.024 | 3.989 |
| KTINGHDSPHKSGQSKQA | 3633 | 101.460 | 0.000 | 0.222 | 0.000 | 0.185 | 0.114 | 7.510 |
| KTSNGSGSPHSKAQNPPT | 3268 | 91.528 | 0.000 | 0.000 | 0.000 | 0.000 | 0.039 | 51.541 |
| ETINGSGSPHSKAQNLQT | 3269 | 90.969 | 0.221 | 1.023 | 0.197 | 0.179 | 0.813 | 107.216 |
| KTVHGNGSPHSKAQNQQT | 3534 | 90.073 | 0.000 | 0.000 | 0.000 | 0.000 | 0.304 | 97.003 |
| NTINGSGSPHSKAQNQQT | 3270 | 90.017 | 1.712 | 1.261 | 1.171 | 0.923 | 0.540 | 55.179 |
| KTINGGGSPHSKAQNQQC | 3535 | 89.301 | 0.219 | 0.000 | 0.000 | 0.287 | 0.319 | 53.840 |
| KTENMSGSPHSKAQNQQT | 3271 | 89.247 | 0.000 | 0.000 | 0.000 | 0.000 | 0.260 | 130.568 |
| KTENVSGSPHSKAQNQQT | 3272 | 88.506 | 0.000 | 0.000 | 0.000 | 0.964 | 0.112 | 108.591 |
| KTSSGSGSPHSKAQYQQT | 3273 | 87.304 | 0.000 | 0.000 | 0.000 | 0.000 | 0.299 | 58.143 |
| KTIDGGGSPHSKAQNKQT | 3536 | 85.019 | 0.000 | 0.000 | 0.000 | 0.000 | 0.477 | 55.517 |
| KTEKVSGSPHSKAQNQQT | 3274 | 84.558 | 0.000 | 0.022 | 0.000 | 0.873 | 0.424 | 112.185 |
| KAINGSGSPHSKAQDQET | 3275 | 84.080 | 0.000 | 0.000 | 0.000 | 0.194 | 0.027 | 87.637 |
| KTCNKSGSPHSKAQNQQT | 3276 | 83.992 | 0.000 | 0.000 | 0.165 | 0.283 | 0.000 | 119.496 |
| KTINGGGSPHSKAQNQLI | 3537 | 83.881 | 0.000 | 0.000 | 0.000 | 0.046 | 0.387 | 78.383 |
| KNINGGGSPHSKAQNQQT | 3538 | 83.083 | 0.000 | 0.042 | 0.000 | 0.000 | 0.000 | 75.913 |
| KTEHLSGSPHSKAQNQQT | 3277 | 83.080 | 0.000 | 0.000 | 0.012 | 0.021 | 0.189 | 69.494 |
| KAIIGHESPHKSGQNQQT | 3634 | 88.563 | 0.000 | 0.150 | 0.000 | 0.062 | 0.145 | 8.530 |
| KTINGHDSPHKTGQNQPP | 3635 | 77.357 | 0.000 | 0.149 | 0.000 | 0.000 | 0.096 | 8.865 |
| KAINGHDSPHKSGQSPQT | 3636 | 75.734 | 0.095 | 0.148 | 0.000 | 0.000 | 0.238 | 14.195 |
| KAEMGSGSPHSKAQNQQT | 3278 | 83.049 | 0.000 | 0.020 | 0.000 | 0.768 | 0.112 | 135.019 |
| KATNGSGSPHSKAQNHQT | 3279 | 82.627 | 0.000 | 0.176 | 0.000 | 0.155 | 0.057 | 66.207 |
| KTIKGNDSPHKSVQNQQT | 3637 | 85.986 | 0.000 | 0.135 | 0.000 | 0.263 | 0.000 | 8.603 |
| KAIKGSGSPHSKAQDQQT | 3280 | 82.258 | 0.000 | 0.000 | 0.000 | 0.108 | 0.000 | 85.178 |
| KTINGGGSPHSKSQNQLT | 3539 | 82.231 | 0.000 | 0.070 | 0.000 | 0.000 | 0.498 | 126.986 |
| KTEFGHDSPHKSGQNQQT | 3638 | 77.245 | 0.000 | 0.124 | 0.000 | 0.561 | 0.063 | 16.337 |
| KTINGHDSPHKSAQNYQT | 3639 | 130.375 | 0.000 | 0.124 | 0.000 | 0.097 | 0.123 | 19.443 |
| KTFNGSASPHSKALNQQT | 3839 | 84.258 | 0.000 | 0.122 | 0.000 | 0.104 | 0.037 | 31.855 |
| KTINGCGSPHASGQNQQT | 3840 | 132.540 | 0.000 | 0.121 | 0.042 | 0.000 | 0.059 | 1.857 |
| KTINAHDSPHKIGQNHQT | 3640 | 106.832 | 0.000 | 0.121 | 0.000 | 0.000 | 0.231 | 5.074 |
| KTVNGNGSPHSKAQNKQT | 3540 | 81.481 | 0.000 | 0.000 | 0.000 | 0.000 | 0.122 | 69.455 |
| KTINGHESPHKSAQNRQT | 3641 | 95.531 | 0.000 | 0.113 | 0.000 | 0.130 | 0.082 | 4.815 |
| KTINGSGSPHSKGHWQQT | 3281 | 81.434 | 0.000 | 0.000 | 0.000 | 0.000 | 1.011 | 65.252 |
| KTTNGHDSPHKSGQNQQG | 3642 | 85.113 | 0.000 | 0.107 | 0.000 | 0.000 | 0.017 | 10.555 |
| KTIKGQDSPHKIGQNQQT | 3643 | 110.357 | 0.000 | 0.103 | 0.058 | 0.166 | 0.135 | 11.829 |
| KTDKTSGSPHSKAQNQQT | 3282 | 81.430 | 0.000 | 0.000 | 0.000 | 1.362 | 0.291 | 169.515 |
| KTVNGHDSPHKSGQNHLT | 3644 | 81.516 | 0.000 | 0.100 | 0.017 | 0.000 | 0.028 | 16.096 |
| KTFKGSGSPHSKAPNQQT | 3283 | 80.890 | 0.000 | 0.000 | 0.000 | 0.000 | 0.017 | 71.144 |
| KSINGHDSPHKSGQYQHT | 3645 | 88.195 | 0.000 | 0.099 | 0.000 | 0.000 | 0.149 | 14.485 |
| KTINGNDSPHKSVQNHQT | 3646 | 120.002 | 0.000 | 0.099 | 0.788 | 0.000 | 0.000 | 7.920 |
| KTVNGSGSPHSKAQNQLI | 3284 | 80.509 | 0.000 | 0.000 | 0.000 | 0.000 | 0.166 | 71.156 |
| KTINGSGSPHSKRPEQQT | 3285 | 80.418 | 0.000 | 0.013 | 0.000 | 0.149 | 0.361 | 50.319 |
| KTINGSGSPHSKAQRTMT | 3286 | 80.388 | 0.000 | 0.022 | 0.170 | 1.812 | 1.025 | 100.248 |
| KTITGHDSPHKSGQNQWT | 3647 | 81.658 | 0.000 | 0.090 | 0.000 | 0.936 | 0.000 | 7.744 |
| KTNNGHDSPHKSVQNQHT | 3648 | 115.172 | 0.000 | 0.083 | 0.000 | 0.000 | 0.062 | 7.934 |
| KTEKASGSPHSKAQNQQT | 3287 | 80.285 | 0.000 | 0.041 | 0.000 | 0.000 | 0.261 | 90.390 |
| KTIDGHDSPHKSGQNQHA | 3649 | 91.058 | 0.000 | 0.082 | 0.000 | 0.000 | 0.000 | 10.781 |
| KSDQGSGSPHSKAQNQQT | 3288 | 80.076 | 0.000 | 0.000 | 0.000 | 0.993 | 0.124 | 151.911 |
| KTVNGHDSPHKSGQTRQT | 3650 | 133.276 | 0.251 | 0.080 | 0.093 | 0.034 | 0.129 | 7.174 |
| KTVNGHDSPHKSGQNLHT | 3651 | 88.080 | 0.000 | 0.080 | 0.000 | 0.000 | 0.039 | 11.363 |
| KAISGHDSPHKSGLNQQT | 3652 | 78.846 | 0.000 | 0.079 | 0.000 | 0.000 | 0.015 | 11.045 |
| KTEITSGSPHSKAQNQQT | 3289 | 79.620 | 0.000 | 0.163 | 0.000 | 0.332 | 0.074 | 76.686 |
| KTDKSSGSPHSKAQNQQT | 3290 | 79.470 | 0.055 | 0.012 | 0.000 | 1.437 | 0.367 | 141.351 |
| KAINGHDSPHKSAQNQET | 3653 | 90.402 | 0.000 | 0.073 | 0.000 | 0.746 | 0.000 | 10.674 |
| KTITGHDSPHKSGQHLQT | 3654 | 137.945 | 0.000 | 0.072 | 0.000 | 0.000 | 0.000 | 4.187 |
| KTIDGSGSPHSKAQNQQH | 3291 | 79.090 | 0.000 | 0.000 | 0.000 | 0.136 | 0.049 | 57.914 |
| KTVNGNGSPHSKAQNQHT | 3541 | 78.849 | 0.000 | 0.000 | 0.000 | 0.000 | 0.045 | 54.086 |
| KNTNGSGSPHSKAQNQQT | 3292 | 78.445 | 0.000 | 0.000 | 0.000 | 0.571 | 0.177 | 89.719 |
| KTINGHDSPHKSRLNQPT | 3655 | 92.883 | 0.000 | 0.070 | 0.050 | 0.904 | 1.075 | 5.598 |
| KTETHSGSPHSKAQNQQT | 3293 | 77.974 | 0.000 | 0.067 | 0.000 | 0.000 | 0.512 | 57.287 |
| KTVDGHDSPHKSGQKQQT | 3656 | 78.802 | 0.000 | 0.069 | 0.000 | 0.157 | 0.342 | 7.200 |
| KTINGQDSPHKSGQNQDT | 3657 | 82.075 | 0.000 | 0.067 | 0.000 | 0.225 | 0.144 | 9.626 |
| KTINGGGSPHSKALNQQN | 3542 | 77.822 | 0.000 | 0.131 | 0.000 | 0.000 | 0.274 | 69.884 |
| KTIEGHDSPHKSGRNQQT | 3658 | 75.838 | 0.000 | 0.065 | 0.017 | 0.000 | 0.079 | 7.818 |
| KTTNGHDSPHKSGQNLLT | 3659 | 77.738 | 0.130 | 0.064 | 0.185 | 0.424 | 0.326 | 15.192 |
| KTINGHDSPHKSGQLVIT | 3660 | 76.781 | 0.089 | 0.064 | 0.000 | 0.338 | 0.475 | 11.323 |
| KTVNGHDSPHKSRQSQQT | 3661 | 76.458 | 0.000 | 0.063 | 0.000 | 0.000 | 0.021 | 8.136 |
| KTINGSGSPHSKALHQHT | 3294 | 77.502 | 0.000 | 0.052 | 0.041 | 0.000 | 0.188 | 68.196 |
| KTINGHDSPHKSGRTQET | 3662 | 81.599 | 0.000 | 0.062 | 0.000 | 0.000 | 0.137 | 7.270 |
| KTINGHDSPHKSVQTHQT | 3663 | 77.309 | 0.237 | 0.062 | 0.000 | 0.000 | 0.116 | 7.519 |
| KTINGTGSPHSKAQNHQI | 3543 | 77.089 | 0.171 | 0.000 | 0.000 | 0.000 | 0.166 | 54.281 |
| KTINGSGSPHSKAQHRIT | 3295 | 76.849 | 0.105 | 0.499 | 0.170 | 1.424 | 0.214 | 127.000 |
| KTINGSGSPHSKAQYIHT | 3296 | 76.170 | 0.000 | 0.014 | 0.033 | 1.523 | 0.168 | 59.649 |
| KTSNGHDSPHKSGQNQPA | 3664 | 75.834 | 0.000 | 0.056 | 0.000 | 0.000 | 0.000 | 8.501 |
| KTEGKHDSPHKSGQNQQT | 3665 | 98.384 | 0.000 | 0.056 | 0.000 | 0.000 | 0.000 | 10.345 |
| KTENISGSPHSKAQNQQT | 3297 | 76.072 | 0.000 | 0.000 | 0.000 | 0.115 | 0.132 | 83.118 |
| KVINGHDSPHKSGQTQQT | 3666 | 91.665 | 0.000 | 0.055 | 1.526 | 0.311 | 0.000 | 7.391 |
| KTIIGGGSPHSKAHNQQT | 3544 | 75.872 | 0.000 | 0.050 | 0.000 | 0.000 | 0.235 | 65.492 |
| KTINGPDSPHKIGQNQQS | 3667 | 85.726 | 0.000 | 0.055 | 0.171 | 0.000 | 0.063 | 10.055 |
| KTINGSGSPHSKAQKFET | 3298 | 75.788 | 0.000 | 0.000 | 0.028 | 0.108 | 0.093 | 65.588 |
| KTSNESGSPHSKAQNHQT | 3299 | 75.720 | 0.000 | 0.000 | 0.000 | 0.169 | 0.217 | 70.590 |
| KTINGSGSPHSKAQFPST | 3300 | 75.677 | 0.000 | 0.004 | 0.000 | 0.849 | 0.127 | 119.712 |
| KTERPSGSPHSKAQNQQT | 3301 | 75.669 | 0.000 | 0.029 | 0.000 | 0.000 | 0.156 | 73.894 |
| KAVNGHDSPHKSVQNQQT | 3668 | 81.051 | 0.448 | 0.051 | 0.000 | 0.665 | 0.091 | 11.288 |
| KTINGNGSPHSKAQNPLT | 3545 | 75.269 | 0.000 | 0.000 | 0.000 | 0.366 | 0.000 | 53.583 |
| KSIKGNGSPHSKAQNQQT | 3546 | 75.196 | 0.000 | 0.000 | 0.000 | 0.000 | 0.000 | 90.251 |
| KTINGHDSPHKSRQDQHT | 3669 | 75.595 | 0.000 | 0.049 | 0.118 | 0.030 | 0.045 | 8.540 |
| KAINGPDSPHKSGQKQQT | 3670 | 78.213 | 0.464 | 0.047 | 0.000 | 0.323 | 0.162 | 10.395 |
| KTINGHDSPHKSRQSQHT | 3671 | 88.544 | 0.499 | 0.046 | 0.000 | 0.059 | 0.032 | 8.324 |
| KTIYGHDSPHKSVQNQLT | 3672 | 92.381 | 0.000 | 0.043 | 0.000 | 0.103 | 0.016 | 12.323 |
| KTVNGHDSPHKSGQNLLT | 3673 | 83.969 | 0.114 | 0.040 | 0.023 | 0.000 | 0.035 | 18.894 |
| KTESAHDSPHKSGQNQQT | 3674 | 80.810 | 0.000 | 0.039 | 0.000 | 0.000 | 0.000 | 13.338 |
| KTENKSGSPHSKAQNQQT | 3594 | 103.854 | 0.000 | 0.037 | 0.000 | 0.000 | 0.119 | 31.182 |
| KTTNGQDSPHKSGQNQQS | 3675 | 92.419 | 0.000 | 0.037 | 0.043 | 0.000 | 0.079 | 7.592 |
| KTDKGSGSPHSKAQNQQT | 3595 | 94.572 | 0.000 | 0.037 | 0.000 | 0.951 | 0.367 | 47.888 |
| KTIDGHDSPHKSGRNQQI | 3676 | 80.240 | 0.000 | 0.037 | 0.000 | 0.040 | 0.144 | 10.363 |
| KTINGYDSPHKSGQYQHT | 3677 | 81.534 | 0.000 | 0.036 | 0.000 | 0.000 | 0.000 | 10.524 |
| KTDNGHDSPHKSRQNQQT | 3678 | 105.312 | 0.000 | 0.033 | 0.000 | 0.000 | 0.018 | 7.931 |
| KTINGHDSPHKSWVRQQT | 3679 | 125.537 | 0.000 | 0.033 | 0.000 | 0.291 | 0.174 | 11.687 |
| KTINGHESPHKSGQNQHS | 3680 | 92.248 | 0.000 | 0.032 | 0.012 | 0.090 | 0.088 | 9.720 |
| KTVNGHDSPHKIGHNQQT | 3681 | 120.985 | 0.000 | 0.029 | 0.000 | 0.000 | 0.009 | 10.167 |
| KTCNGHDSPHKSGRNQQT | 3682 | 94.616 | 0.000 | 0.025 | 0.000 | 0.000 | 0.128 | 12.496 |
| KTINGNGSPHSKAQNHQA | 3841 | 88.274 | 0.000 | 0.024 | 0.000 | 0.000 | 0.041 | 36.754 |
| KNVVGHDSPHKSGQNQQT | 3683 | 75.330 | 0.000 | 0.024 | 0.000 | 0.063 | 0.049 | 8.077 |
| KTELWHDSPHKSGQNQQT | 3684 | 85.323 | 0.057 | 0.020 | 0.000 | 0.000 | 0.243 | 9.915 |
| KTELRHDSPHKSGQNQQT | 3685 | 98.098 | 0.000 | 0.019 | 0.000 | 0.000 | 0.007 | 6.588 |
| KTINGHDSPHKSNAWQQT | 3686 | 84.825 | 0.000 | 0.016 | 0.000 | 0.000 | 0.132 | 15.788 |
| KTDAGHDSPHKSGQNQQT | 3687 | 88.924 | 0.000 | 0.013 | 0.000 | 1.076 | 0.070 | 18.107 |
| KTEVGHDSPHKSGQNQQT | 3688 | 112.457 | 0.000 | 0.011 | 0.000 | 0.000 | 0.138 | 13.125 |
| KTESRHDSPHKSGQNQQT | 3689 | 81.766 | 0.000 | 0.011 | 0.000 | 0.052 | 0.036 | 6.975 |
| KSELGHDSPHKSGQNQQT | 3690 | 107.059 | 0.000 | 0.005 | 0.000 | 0.000 | 0.055 | 13.285 |
| KTINGHDSPHKSGQSVPT | 3691 | 77.840 | 0.000 | 0.003 | 0.000 | 0.136 | 0.061 | 6.768 |
| KTINGHESPHKSGQNIQP | 3692 | 253.840 | 0.000 | 0.000 | 0.000 | 0.000 | 0.000 | 14.042 |
| KTEMKHDSPHKSGQNQQT | 3693 | 240.075 | 0.000 | 0.000 | 0.000 | 0.000 | 0.000 | 3.183 |
| KTINGHDSPHKSVQNHLN | 3694 | 196.758 | 0.000 | 0.000 | 0.000 | 0.000 | 0.000 | 14.557 |
| KTINGHDSPHKIGLDQQT | 3695 | 165.627 | 0.000 | 0.000 | 0.000 | 1.942 | 0.000 | 5.469 |
| KTSNASGSPHSKAQHQQT | 3596 | 165.206 | 0.000 | 0.000 | 0.000 | 0.000 | 0.082 | 40.558 |
| KTINGHDSPHKRGPDQQS | 3696 | 160.084 | 0.000 | 0.000 | 0.000 | 0.000 | 0.000 | 2.923 |
| KTINGMGSPHSKTQNQQT | 3842 | 158.728 | 0.000 | 0.000 | 0.000 | 0.000 | 0.638 | 47.809 |
| KTIKGHDSPHKSGESQQT | 3697 | 142.264 | 0.000 | 0.000 | 0.000 | 0.000 | 0.218 | 4.176 |
| KTEGWHDSPHKSGQNQQT | 3698 | 142.064 | 0.000 | 0.000 | 0.000 | 0.000 | 0.264 | 11.785 |
| KTINGHDSPHKHGQNHQT | 3699 | 141.405 | 0.191 | 0.000 | 0.000 | 0.000 | 0.000 | 10.214 |
| KTEQLHDSPHKSGQNQQT | 3700 | 138.345 | 0.000 | 0.000 | 0.000 | 0.000 | 0.000 | 12.606 |
| KTVNGTGSPHSKAQNQLT | 3843 | 137.639 | 0.000 | 0.000 | 0.000 | 0.000 | 0.277 | 48.950 |
| KTIIGHDSPHKSGQYQHT | 3701 | 131.825 | 0.000 | 0.000 | 0.000 | 0.000 | 0.211 | 5.762 |
| KTSNGHDSPHKSVQNKQT | 3702 | 130.640 | 0.000 | 0.000 | 0.000 | 0.172 | 0.039 | 11.850 |
| KIVNGQVSPHKSGQNQQT | 3703 | 129.649 | 0.000 | 0.000 | 0.000 | 0.000 | 0.031 | 16.942 |
| KTVNGHDSPHKSGQRQLT | 3704 | 129.641 | 0.000 | 0.000 | 0.000 | 0.000 | 0.487 | 20.145 |
| KTVNGHDSPHKIGQNQLT | 3705 | 128.582 | 0.000 | 0.000 | 0.499 | 0.027 | 0.199 | 20.957 |
| KTINGHDSPHKSGQIIVT | 3706 | 125.245 | 0.000 | 0.000 | 0.151 | 0.000 | 0.379 | 6.808 |
| KTEKIHDSPHKSGQNQQT | 3707 | 125.178 | 0.000 | 0.000 | 0.000 | 0.000 | 0.000 | 17.604 |
| KTENAHDSPHKSGQNQQT | 3708 | 124.477 | 0.000 | 0.000 | 0.000 | 0.000 | 0.062 | 15.805 |
| KIGNGHESPHKSGQNQQT | 3709 | 123.324 | 0.000 | 0.000 | 0.000 | 0.000 | 0.000 | 11.198 |
| KEVMGHDSPHKSGQNQQT | 3710 | 121.107 | 0.000 | 0.000 | 0.000 | 0.000 | 0.000 | 17.191 |
| KTEVKHDSPHKSGQNQQT | 3711 | 119.733 | 0.000 | 0.000 | 0.000 | 0.000 | 0.000 | 5.550 |
| KTINGYDSPHKSGQKQST | 3712 | 119.615 | 0.000 | 0.000 | 0.000 | 0.000 | 0.000 | 7.970 |
| KTIHGNGSPHSKAQNQET | 3844 | 117.388 | 0.000 | 0.000 | 0.000 | 0.000 | 0.000 | 38.874 |
| KYQVGHDSPHKSGQNQQT | 3713 | 112.797 | 0.000 | 0.000 | 0.000 | 0.000 | 0.542 | 9.335 |
| KTEAMHDSPHKSGQNQQT | 3714 | 111.765 | 0.000 | 0.000 | 0.000 | 0.000 | 0.000 | 16.142 |
| KTIKGDDSPHKSVQNQQT | 3715 | 109.397 | 0.000 | 0.000 | 0.000 | 0.000 | 0.000 | 19.125 |
| KTINGHDSPHKSVQSHQT | 3716 | 109.375 | 0.107 | 0.000 | 0.319 | 0.000 | 0.547 | 12.617 |
| KTINGHDSPHKSGQFVVT | 3717 | 108.725 | 0.000 | 0.000 | 0.000 | 0.124 | 0.406 | 10.179 |
| KTVNGHDSPHKSRQNLQT | 3718 | 107.496 | 0.205 | 0.000 | 0.000 | 1.934 | 0.062 | 8.616 |
| KATNGHNSPHKSGQNQET | 3719 | 106.806 | 0.000 | 0.000 | 0.000 | 0.000 | 0.000 | 10.566 |
| KAINGHDSPHKSAQNQQI | 3720 | 106.539 | 0.000 | 0.000 | 0.000 | 0.000 | 0.113 | 21.786 |
| KTEHGHDSPHKSGQNQQT | 3721 | 106.486 | 0.000 | 0.000 | 0.000 | 0.000 | 0.006 | 14.956 |
| KTVENHDSPHKSGQNQQT | 3722 | 106.468 | 0.000 | 0.000 | 0.000 | 0.000 | 0.156 | 9.246 |
| KTIYGHDSPHKSGQSQPT | 3723 | 106.431 | 0.000 | 0.000 | 0.000 | 0.155 | 0.137 | 6.562 |
| KTISGHESPHKSGQNEQT | 3724 | 105.740 | 0.000 | 0.000 | 0.000 | 0.378 | 1.384 | 9.156 |
| KAIIGHDSPHKSAQNQQT | 3725 | 105.292 | 0.000 | 0.000 | 0.000 | 0.000 | 0.553 | 16.793 |
| KAIDGHDSPHKSGQNQLT | 3726 | 104.701 | 0.331 | 0.000 | 0.000 | 0.201 | 0.638 | 16.109 |
| KTIMGHDSPHKSVQNQQT | 3727 | 104.683 | 0.000 | 0.000 | 0.000 | 0.000 | 0.000 | 8.029 |
| KEVGGHDSPHKSGQNQQT | 3728 | 103.896 | 0.000 | 0.000 | 0.000 | 0.000 | 0.000 | 16.899 |
| KTINGHDSPHKSAQNLLT | 3729 | 103.332 | 0.000 | 0.000 | 0.256 | 0.194 | 0.000 | 15.722 |
| KTEFTHDSPHKSGQNQQT | 3730 | 102.052 | 0.062 | 0.000 | 0.000 | 0.439 | 0.047 | 12.527 |
| KTINASGSPHSKAINQQT | 3597 | 101.122 | 0.233 | 0.000 | 0.000 | 0.000 | 0.145 | 47.196 |
| KAINGNGSPHKRGQNQQT | 3845 | 100.925 | 0.000 | 0.000 | 0.000 | 0.000 | 0.159 | 10.011 |
| KSEMGHDSPHKSGQNQQT | 3731 | 100.539 | 0.000 | 0.000 | 0.000 | 0.000 | 0.000 | 18.356 |
| KAQQGHDSPHKSGQNQQT | 3732 | 100.395 | 0.000 | 0.000 | 0.000 | 0.000 | 0.057 | 3.954 |
| KTEVMHDSPHKSGQNQQT | 3733 | 99.473 | 0.000 | 0.000 | 0.000 | 0.000 | 0.000 | 12.400 |
| KAINGHDSPHKSGQSLQT | 3734 | 99.310 | 0.058 | 0.000 | 1.439 | 0.254 | 0.056 | 17.323 |
| KTINGSGSPHSKAPNQQH | 3598 | 99.300 | 0.252 | 0.000 | 0.000 | 0.000 | 0.038 | 39.297 |
| KCGEGHDSPHKSGQNQQT | 3735 | 99.298 | 0.000 | 0.000 | 0.000 | 0.000 | 0.000 | 13.147 |
| KTVNGHDSPHKSAQNHQT | 3736 | 99.257 | 0.000 | 0.000 | 0.078 | 0.000 | 0.027 | 17.639 |
| KTVNGHDSPHKSGQTQLT | 3737 | 98.524 | 0.000 | 0.000 | 0.313 | 0.183 | 0.172 | 14.883 |
| KTNNGHDSPHKSGRNRQT | 3738 | 98.307 | 0.000 | 0.000 | 0.124 | 0.000 | 0.037 | 5.840 |
| KTCNEHDSPHKSGQNQQT | 3739 | 97.092 | 0.000 | 0.000 | 0.000 | 0.000 | 0.000 | 8.364 |
| KTINGHDSPHKYGQNEQT | 3740 | 96.960 | 0.000 | 0.000 | 0.000 | 0.000 | 0.000 | 4.613 |
| KASNRHDSPHKSGHNQQT | 3741 | 96.283 | 0.000 | 0.000 | 0.000 | 0.000 | 0.675 | 8.045 |
| KTINGNGSPHSKAPNLQT | 3846 | 95.963 | 0.000 | 0.000 | 0.000 | 0.000 | 0.247 | 36.341 |
| KTETKHDSPHKSGQNQQT | 3742 | 95.121 | 0.000 | 0.000 | 0.000 | 0.000 | 0.083 | 4.831 |
| KSINGHDSPHKSQQNQQT | 3743 | 94.479 | 0.000 | 0.000 | 0.000 | 0.000 | 1.696 | 9.633 |
| KTIGGHDSPHKSGQNQQI | 3744 | 94.420 | 0.000 | 0.000 | 0.000 | 0.000 | 0.333 | 19.324 |
| KTDPQHDSPHKSGQNQQT | 3745 | 93.931 | 0.000 | 0.000 | 0.000 | 0.906 | 0.019 | 11.749 |
| KTINRHDSPHKIVQNQQT | 3746 | 93.409 | 0.000 | 0.000 | 0.000 | 0.000 | 0.000 | 3.064 |
| KTEQYHDSPHKSGQNQQT | 3747 | 93.065 | 0.000 | 0.000 | 0.000 | 0.000 | 0.047 | 15.190 |
| KTINGHDSPHKSVQSKQT | 3748 | 92.445 | 0.000 | 0.000 | 0.078 | 0.000 | 0.047 | 4.263 |
| KELVGHDSPHKSGQNQQT | 3749 | 92.262 | 0.000 | 0.000 | 0.000 | 0.594 | 0.000 | 14.890 |
| KTENRHDSPHKSGQNQQT | 3750 | 91.675 | 0.000 | 0.000 | 0.000 | 0.000 | 0.000 | 13.282 |
| KELMGHDSPHKSGQNQQT | 3751 | 91.191 | 0.000 | 0.000 | 0.000 | 0.000 | 0.000 | 14.032 |
| KTINGNDSPHKIGHNQQT | 3752 | 91.183 | 0.000 | 0.000 | 0.117 | 0.000 | 0.270 | 11.095 |
| KTIKGGGSPHSKAQDQQT | 3847 | 91.172 | 0.000 | 0.000 | 0.000 | 0.064 | 0.085 | 49.580 |
| KTEGHHDSPHKSGQNQQT | 3753 | 89.922 | 0.000 | 0.000 | 0.000 | 0.000 | 0.000 | 23.929 |
| KTEGYHDSPHKSGQNQQT | 3754 | 89.891 | 0.000 | 0.000 | 0.000 | 0.000 | 0.000 | 15.116 |
| KTVNGHDSPHKSGQTQQI | 3755 | 89.801 | 0.000 | 0.000 | 0.657 | 0.000 | 0.542 | 11.543 |
| KTINGQDSPHKSGQNPLT | 3756 | 89.726 | 0.000 | 0.000 | 0.000 | 0.363 | 0.000 | 15.561 |
| KTVNASGSPHSKAQNHQT | 3599 | 89.467 | 0.091 | 0.000 | 0.000 | 0.045 | 0.252 | 39.024 |
| KTINGHDSPHKSGRDQKT | 3757 | 88.871 | 0.000 | 0.000 | 0.000 | 0.350 | 0.181 | 12.117 |
| KTINGHDSPHKSVHNQQN | 3758 | 88.715 | 0.089 | 0.000 | 0.081 | 0.000 | 0.143 | 10.787 |
| KTINGHDSPHKSGQWKRT | 3759 | 88.633 | 0.000 | 0.000 | 0.000 | 0.202 | 0.094 | 5.186 |
| KTIDGSGSPHSKAENRQT | 3600 | 87.993 | 0.092 | 0.000 | 0.000 | 0.139 | 0.054 | 40.629 |
| KNEIGHDSPHKSGQNQQT | 3760 | 87.758 | 0.000 | 0.000 | 0.000 | 0.000 | 0.055 | 14.110 |
| KAINGHDSPHKSGQSQQI | 3761 | 87.585 | 0.000 | 0.000 | 5.310 | 0.000 | 0.000 | 12.864 |
| KIINGHDSPHKSRQAQQT | 3762 | 86.966 | 0.000 | 0.000 | 0.000 | 0.000 | 0.000 | 9.193 |
| KTPNGHDSPHKSGQNQQI | 3763 | 86.683 | 0.000 | 0.000 | 0.000 | 0.000 | 0.109 | 21.278 |
| KITNGHDSPHKSGQTQQT | 3764 | 86.443 | 0.000 | 0.000 | 0.000 | 0.192 | 0.190 | 17.479 |
| KTINGHDSPHKSVQNHQI | 3765 | 86.395 | 0.000 | 0.000 | 0.000 | 0.000 | 0.000 | 9.148 |
| KTINGHDSPHKSKQNQQA | 3766 | 86.265 | 0.000 | 0.000 | 0.000 | 0.123 | 0.041 | 5.768 |
| KTINGHDSPHKSAQNQLN | 3767 | 86.153 | 0.000 | 0.000 | 0.000 | 0.050 | 0.019 | 15.587 |
| KTDITHDSPHKSGQNQQT | 3768 | 85.876 | 0.000 | 0.000 | 0.000 | 0.000 | 0.013 | 9.076 |
| KTVNGHDSPHKSGQTQPT | 3769 | 85.680 | 0.000 | 0.000 | 1.301 | 1.064 | 0.000 | 8.067 |
| KTEKFHDSPHKSGQNQQT | 3770 | 85.358 | 0.000 | 0.000 | 0.000 | 0.000 | 0.026 | 7.229 |
| KTDQGHDSPHKSGQNQQT | 3771 | 85.267 | 0.000 | 0.000 | 0.000 | 0.000 | 0.000 | 16.042 |
| KTINGHDSPHKLWINQQT | 3772 | 85.132 | 0.000 | 0.000 | 1.154 | 0.000 | 0.017 | 12.704 |
| KGINGPDSPHKSGQNQQT | 3773 | 85.080 | 0.000 | 0.000 | 0.084 | 0.000 | 0.054 | 13.750 |
| KSEIGHDSPHKSGQNQQT | 3774 | 84.789 | 0.000 | 0.000 | 0.000 | 0.000 | 0.013 | 15.955 |
| KTINGHDSPHKSVQKQLT | 3775 | 84.351 | 0.000 | 0.000 | 0.000 | 0.038 | 0.103 | 11.890 |
| KTINGHPSPHWKGQNQQT | 3848 | 84.153 | 0.000 | 0.000 | 0.000 | 0.000 | 0.058 | 3.280 |
| KTVNGHDSPHKSGRNQLA | 3776 | 83.858 | 0.000 | 0.000 | 0.000 | 0.000 | 0.132 | 21.252 |
| KTNNVHDSPHKSGQNQQS | 3777 | 83.697 | 0.000 | 0.000 | 0.000 | 0.176 | 0.000 | 7.117 |
| KTIKGSGSPHSKVQDQQT | 3601 | 83.077 | 0.000 | 0.000 | 0.034 | 0.000 | 0.107 | 21.001 |
| KSEKGHDSPHKSGQNQQT | 3778 | 82.982 | 0.000 | 0.000 | 0.000 | 0.000 | 0.105 | 16.662 |
| KWSAGHDSPHKSGQNQQT | 3779 | 82.949 | 0.000 | 0.000 | 0.000 | 0.000 | 0.211 | 12.499 |
| KELAGHDSPHKSGQNQQT | 3780 | 82.876 | 0.000 | 0.000 | 0.000 | 0.000 | 0.093 | 18.063 |
| KTINGHDSPHKMGRNQQS | 3781 | 82.787 | 0.000 | 0.000 | 0.000 | 0.000 | 0.000 | 6.467 |
| KTDQAHDSPHKSGQNQQT | 3782 | 82.402 | 0.000 | 0.000 | 0.141 | 0.000 | 0.000 | 13.397 |
| KTETQHDSPHKSGQNQQT | 3783 | 82.316 | 0.000 | 0.000 | 0.000 | 0.000 | 0.198 | 10.823 |
| KTEMTHDSPHKSGQNQQT | 3784 | 82.221 | 0.000 | 0.000 | 0.000 | 0.000 | 0.000 | 8.431 |
| KTINGHDSPHKSGISIQT | 3785 | 82.019 | 0.000 | 0.000 | 0.000 | 0.191 | 0.044 | 7.310 |
| KTDAVHDSPHKSGQNQQT | 3786 | 81.968 | 0.000 | 0.000 | 0.000 | 0.297 | 0.107 | 13.596 |
| KTSNGHDSPHKSVQNLQT | 3787 | 81.921 | 0.000 | 0.000 | 0.072 | 0.000 | 0.330 | 11.544 |
| KTEKYHDSPHKSGQNQQT | 3788 | 81.637 | 0.000 | 0.000 | 0.000 | 0.000 | 0.013 | 7.580 |
| KQTQGHDSPHKSGQNQQT | 3789 | 81.581 | 0.000 | 0.000 | 0.000 | 0.000 | 0.133 | 15.225 |
| KTINGHDSPHKMAHNQQT | 3790 | 81.329 | 0.000 | 0.000 | 0.000 | 0.000 | 0.094 | 15.949 |
| KAINGSGSPHSKAQTQQA | 3602 | 81.207 | 0.000 | 0.000 | 0.000 | 0.000 | 0.016 | 40.435 |
| KTINGHDSPHKHGQNQQN | 3791 | 81.065 | 0.000 | 0.000 | 0.000 | 0.000 | 0.000 | 4.110 |
| KGADGHDSPHKSGQNQQT | 3792 | 80.981 | 0.000 | 0.000 | 0.000 | 0.000 | 0.074 | 11.423 |
| KVGEGHDSPHKSGQNQQT | 3793 | 80.775 | 0.084 | 0.000 | 0.000 | 0.000 | 0.019 | 16.378 |
| KANEGHDSPHKSGQNQQT | 3794 | 80.470 | 0.000 | 0.000 | 0.000 | 0.000 | 0.000 | 12.818 |
| KTDTMHDSPHKSGQNQQT | 3795 | 80.364 | 0.000 | 0.000 | 0.000 | 0.000 | 0.000 | 13.166 |
| KTEAKSGSPHSKAQNQQT | 3603 | 80.088 | 0.192 | 0.000 | 0.000 | 0.000 | 0.613 | 47.130 |
| KTINGHDSPHKSVQSQQS | 3796 | 80.000 | 0.000 | 0.000 | 0.000 | 1.055 | 0.082 | 17.620 |
| KTIPGSGSPHSKAQNLQT | 3604 | 79.973 | 0.871 | 0.000 | 0.000 | 0.000 | 0.000 | 32.693 |
| KTCIAHDSPHKSGQNQQT | 3797 | 79.857 | 0.000 | 0.000 | 0.066 | 0.000 | 0.093 | 1.930 |
| KTINGHDSPHKSGQTVCT | 3798 | 79.730 | 0.000 | 0.000 | 0.000 | 0.050 | 0.030 | 7.873 |
| KELRGHDSPHKSGQNQQT | 3799 | 79.596 | 0.000 | 0.000 | 0.000 | 0.000 | 0.006 | 22.001 |
| KCQIGHDSPHKSGQNQQT | 3800 | 79.359 | 0.000 | 0.000 | 0.000 | 0.000 | 0.000 | 2.614 |
| KGVMGHDSPHKSGQNQQT | 3801 | 79.170 | 0.000 | 0.000 | 0.000 | 0.138 | 0.086 | 17.287 |
| KACDGHDSPHKSGQNQQT | 3802 | 78.648 | 0.000 | 0.000 | 0.000 | 0.000 | 0.128 | 17.767 |
| KTINGQDSPHKSGQYQQI | 3803 | 78.585 | 0.000 | 0.000 | 0.000 | 0.286 | 0.672 | 5.664 |
| KTINGHDSPHKSGQQIMT | 3804 | 78.534 | 0.000 | 0.000 | 0.000 | 0.000 | 0.058 | 7.067 |
| KTINGHDSPHKSRQNEQS | 3805 | 78.534 | 0.000 | 0.000 | 0.000 | 0.112 | 0.188 | 13.388 |
| KASNGHDSPHKSGLNHQT | 3806 | 78.451 | 0.000 | 0.000 | 0.000 | 0.000 | 0.000 | 17.975 |
| KTVNGHDSPHKSGQSQPT | 3807 | 78.309 | 0.000 | 0.000 | 0.000 | 0.000 | 0.231 | 10.627 |
| KNELGHDSPHKSGQNQQT | 3808 | 78.135 | 0.000 | 0.000 | 0.000 | 0.000 | 0.182 | 17.457 |
| KTETFHDSPHKSGQNQQT | 3809 | 78.070 | 0.000 | 0.000 | 0.000 | 0.782 | 0.007 | 4.693 |
| KAAEGHDSPHKSGQNQQT | 3810 | 77.793 | 0.000 | 0.000 | 0.000 | 0.000 | 0.060 | 13.552 |
| KGQNGHDSPHKSGQNQQT | 3811 | 77.770 | 0.000 | 0.000 | 0.000 | 0.107 | 0.056 | 13.618 |
| KNEFGHDSPHKSGQNQQT | 3812 | 77.740 | 0.000 | 0.000 | 0.000 | 0.000 | 0.029 | 16.318 |
| KTSIGYDSPHKSGQNQQT | 3813 | 77.730 | 0.000 | 0.000 | 0.000 | 0.057 | 0.178 | 4.831 |
| KTDNGHDSPHKSGQNLQT | 3814 | 77.565 | 0.504 | 0.000 | 0.000 | 0.000 | 0.000 | 16.184 |
| KTEGQHDSPHKSGQNQQT | 3815 | 77.423 | 0.000 | 0.000 | 0.000 | 0.000 | 0.748 | 20.310 |
| KTITGHDSPHKSRQDQQT | 3816 | 77.127 | 0.000 | 0.000 | 0.000 | 0.000 | 0.000 | 6.250 |
| KAEHGHDSPHKSGQNQQT | 3817 | 77.026 | 0.000 | 0.000 | 0.000 | 0.000 | 0.017 | 20.937 |
| KTINGDDSPHKSGQKQLT | 3818 | 76.968 | 0.000 | 0.000 | 0.000 | 0.163 | 0.014 | 15.820 |
| KCDQGHDSPHKSGQNQQT | 3819 | 76.887 | 0.000 | 0.000 | 0.000 | 0.193 | 0.013 | 27.317 |
| KEILGHDSPHKSGQNQQT | 3820 | 76.770 | 0.000 | 0.000 | 0.000 | 0.804 | 0.009 | 10.771 |
| KTIHGSGSPHSKAQNQAT | 3605 | 76.765 | 0.000 | 0.000 | 0.000 | 0.000 | 0.215 | 43.969 |
| KTERNHDSPHKSGQNQQT | 3821 | 76.751 | 0.000 | 0.000 | 0.000 | 0.000 | 0.000 | 14.979 |
| KAINGDDSPHKSGHNQQT | 3822 | 76.578 | 0.000 | 0.000 | 0.000 | 0.032 | 0.059 | 17.755 |
| KTSNGHNSPHKSGQNQET | 3823 | 76.515 | 0.000 | 0.000 | 0.000 | 0.000 | 0.000 | 4.764 |
| KTINGHDSPHKSGQMIHT | 3824 | 76.364 | 0.000 | 0.000 | 0.000 | 0.000 | 0.000 | 9.486 |
| KNAIGHDSPHKSGQNQQT | 3825 | 76.289 | 0.000 | 0.000 | 0.000 | 0.009 | 0.072 | 15.178 |
| KTDKFHDSPHKSGQNQQT | 3826 | 76.204 | 0.000 | 0.000 | 0.000 | 0.000 | 0.000 | 7.096 |
| KTEGFHDSPHKSGQNQQT | 3827 | 76.191 | 0.000 | 0.000 | 0.000 | 0.000 | 0.080 | 13.163 |
| KVINGHDSPHKSGRNHQS | 3828 | 75.961 | 0.000 | 0.000 | 0.000 | 0.000 | 0.000 | 13.568 |
| KTITGHDSPHKSVQNRQT | 3829 | 75.940 | 0.000 | 0.000 | 0.000 | 0.621 | 0.000 | 4.310 |
| KTPDMHDSPHKSGQNQQT | 3830 | 75.871 | 0.659 | 0.000 | 0.000 | 0.000 | 0.048 | 11.277 |
| KTINGHDSPHKSGQKMNT | 3831 | 75.820 | 0.000 | 0.000 | 0.000 | 0.000 | 0.167 | 6.373 |
| KTELQHDSPHKSGQNQQT | 3832 | 75.814 | 0.000 | 0.000 | 0.000 | 0.105 | 0.000 | 11.798 |
| KTIHGHDSPHKSGQSQQN | 3833 | 75.777 | 0.000 | 0.000 | 0.059 | 0.000 | 0.166 | 7.426 |
| KTEIGHDSPHKSGQNQQT | 3834 | 75.525 | 0.000 | 0.000 | 0.016 | 0.012 | 0.000 | 9.593 |
| KTINGHDSPHKSGQYQHA | 3835 | 75.308 | 0.000 | 0.000 | 0.000 | 0.000 | 0.017 | 17.081 |
| KTELYHDSPHKSGQNQQT | 3836 | 75.235 | 0.000 | 0.000 | 0.000 | 0.000 | 0.042 | 10.354 |

Additional variants were identified following generation and screening in NHPs that had the following properties. TTM-001 and TTM-002 capsid variants comprising the amino acid sequence of SEQ ID NOs: 4253, 4281, 429N0-4295, 4304, 4305, 4320, 4328-4335.4337-43.0, 4353, 4355, 4369, 4387, 4421, 4424-4428, 4430, 4432, 4433, 4435, 4436-4449, 4452, 4455, 4476, 4483, or 4484 had a raw virus count 10 or greater, a CV of less than 1 for the brain samples isolated from the NHPs, demonstrated a 50-fold or greater increase in expression in the brain of mice and NHPs relative to AAV9, and demonstrated 2-fold or less expression in the liver and DRG of NHPs relative to AAV9. TTM-001 and TTM-002 capsid variants comprising the amino acid sequence of SEQ ID NOs: 4098-4105, 4254-4280, 4282-4289, 4296-4303, 4306-4327, 4336, 4341-4352, 4354, 4356-4420, 4422, 4423, 4425, 4429, 4431, 4434, 4444, 4450, 4451, 4453, 4454, 4456-4475, 4477-4482, or 4485 had a CV of less than 1 in across the brain samples isolated from the NHPs and demonstrated a 100-fold or greater increase in expression in the brain of NHPs relative to AAV9. TIM-001 and TTM-002 capsid variants comprising the amino acid sequence of SEQ ID NOs: 4102 and 4106-4252 had normalized virus counts of greater than or equal to 0.01, a CV of less than 1 across the liver RNA samples isolated from the NHPs, and demonstrated a 20-fold or greater increase in expression in the liver of NHPs relative to AAV9. TM-001 and TTM-002 capsid variants comprising the amino acid sequence of SEQ ID NO: 4105 had a raw virus count 9.9 or greater, a CV of less than 1 across the muscle samples isolated from the NHPs, and 5-fold or greater increase in expression in the muscle of the NHPs relative to AAV9. TM-001 and TTM-002 capsid variants comprising the amino acid sequence of SEQ ID NO: 4105 also had a raw virus count 9.9 or greater, a CV of less than 1 across the samples isolated from the heart of the NHPs, and 5-fold or greater increase in expression in the heart of the NHPs relative to AAV9.

These additional matured variants were then incorporated into a synthetic library that was passaged through another NHP or mouse along with the TTM-002 capsid variant, the TTM-001 capsid variant, and an AAV9 capsid as controls. Following this passage, CNS and peripheral tissues were collected and RNA was extracted. Following RNA recovery and RT-PCR amplification, a systematic NGS enrichment analysis was performed to calculate the fold enrichment ratio relative to the AAV9 wild-type control or the TTM-002 capsid variant control. The matured capsid variants were filtered first by capsid fitness (virus counts/DNA counts), selecting those samples that had a fitness threshold greater than or equal to 0.75. Following this initial selection, brain tropic and heart tropic matured capsid variants were selected. For brain tropic capsid variants with little to no expression in the liver (liver de-targeted) or heart, the matured capsid variants were further filtered based on a CV of less than 0.5 across the brain RNA samples isolated from the NHPs, a fold-change in DNA expression in the liver of the NHPs of less than 0.2 relative to the AAV9 wild-type control, a ratio of brain RNA to liver DNA in NHPs of at least 100, at least 2.5 or 3 fold-enrichment or greater relative to the TTM-002 control in the brain of the NHPs, at least 0.8 fold-enrichment or greater in the brain of mice relative to the TTM-002 control, and an average fold-change in expression of RNA in the heart relative to the AAV9 control of less than 1.5. The resulting brain tropic matured capsid variants that lead to increased expression in the brain of mice and NHPs comprised the amino acid sequences of SEQ ID NOs: 3272, 3274, 3421, 3487, 3589, and 4478, many of which comprised an E at position 451 numbered according to SEQ ID NOs: 138, 981, or 982 (or the third position numbered according to SEQ ID NOs: 3272, 3274, 3421, 3487, 3589, and 4478), and either an N or a positively charged residue (e.g., K or R) at position 452 numbered according to SEQ ID NO: 138, 981, or 982 (or the fourth position numbered according to SEQ ID NOs: 3272, 3274, 3421, 3487, 3589, and 4478). For heart tropic capsids, the capsid variants were further filtered based on a CV of less than 0.5 across the heart RNA samples isolated from the NHPs, a fold-change in DNA expression in the liver of the NHPs of less than 0.5 relative to the AAV9 wild-type control, and a fold-change in RNA expression of less than 1.5 in the brain of NHPs relative to the TTM-002 control. The resulting heart tropic matured capsid variants comprised the amino sequence of SEQ ID NOs: 3382 and 4406. The data and sequences for the selected brain and heart tropic capsid variants that met the filtering criteria are provided in Table 75. These brain and heart tropic matured capsid variants also demonstrated decreased tropism in the DRG.

TABLE 75
NGS fold-enrichment of TTM-001 and TTM-002 matured AAV capsid variants with
increased expression in the brain or heart of NHPs and mice

| | | Average | | | | | | |
|---|---|---|---|---|---|---|---|---|
| | | Fold | | | | | | |
| | | Enrichment | | | | | | |
| | | relative | | | | | | |
| | | to TTM-002 | Average Fold Enrichment relative to AAV9 | | | | | |
| | | Brain | Brain | Spinal | Liver | Heart | Muscle | Brain |
| | SEQ | RNA | RNA | Cord | DNA | RNA | DNA | RNA |
| Sequence | ID NO | (NHP) | (NHP) | (NHP) | (NHP) | (NHP) | (NHP) | (Mouse) |
| KTENVSGSPHSKAQNQQT | 3272 | 7.333 | 35.140 | 12.982 | 0.088 | 1.242 | 2.077 | 8.619 |
| KTEKVSGSPHSKAQNQQT | 3274 | 6.203 | 29.725 | 3.103 | 0.138 | 1.165 | 0.855 | 6.073 |
| KTERVSGSPHSKAQNQQT | 3589 | 4.147 | 19.870 | 4.171 | 0.123 | 0.872 | 5.141 | 6.080 |
| KNSIGSGSPHSKAQNQQT | 3421 | 3.582 | 17.164 | 3.080 | 0.137 | 0.798 | 0.772 | 7.657 |
| KTVNGHDSPHKSGQRPST | 4478 | 2.890 | 13.848 | 5.763 | 0.110 | 0.551 | 4.544 | 6.833 |
| KAENGSGSPHSKAQNQQT | 3487 | 2.627 | 12.588 | 3.183 | 0.079 | 0.526 | 1.945 | 7.571 |
| KTSNASGSPHSKAHNQQT | 3382 | 1.419 | 6.799 | 8.066 | 0.352 | 6.641 | 7.517 | 8.203 |
| KTINGSGSPHKSGQNKTS | 4406 | 0.550 | 2.637 | 0.655 | 0.317 | 7.899 | 12.738 | 1.932 |
| KTINGSGSPHSKAQNQQT | 7411 | 1.297 | 6.216 | 5.338 | 0.456 | 3.062 | 4.732 | 5.964 |
| KTINGHDSPHKSGQNQQT | 7579 | 1.000 | 4.792 | 2.504 | 0.181 | 2.456 | 4.591 | 7.103 |

These data demonstrate that following two maturation approaches, matured TTM-001 and TTM-002 capsid variants (AAV9 capsid variants) with loop IV modifications were generated with significantly enhanced CNS tropism over wild-type AAV9 controls in both NHPs and mice, while also exhibiting de-targeting in peripheral tissues (e.g., the liver and DRG). These resulting matured variants therefore demonstrated cross-species CNS tropism in both NHPs and mice.

### Example 13. Evaluation of TTM-001 and TTM-002 AAV Capsid Variants in Diverse Primate Species

This Example evaluates the tropism and cross-species compatibility of the TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2) capsid variants in two diverse primate species, marmosets (Callithrix jacchus) and African green monkeys (Chlorocebus sabaeus), as compared to their tropism in cynomolgus macaques (Macaca fascicularis) provided in Example 10. The cross-species compatibility and tropism of an AAV9 capsid variant comprising the amino acid sequence of SPHKYG (SEQ ID NO: 966) was also investigated in this example. The amino acid and DNA sequences of TTM-001 and TTM-002 are provided, e.g., in Tables 4 and 5, respectively.

To investigate tropism in African green monkeys, AAV particles comprising the TTM-001 capsid variant, the TTM-002 capsid variant, an AAV9 capsid variant comprising SEQ ID NO: 966, or an AAV9 control under the control of a synapsin promoter, were intravenously injected into NHPs (n=2, 3-12 years of age) at a dose of 2E13 vg/kg. After 14-days in life, the brains and tissues (liver, DRG, quadriceps, and heart) of the NHPs were collected and RNA was extracted. Following RNA recovery and RT-PCR amplification, a systematic NGS enrichment analysis was performed to calculate the fold enrichment ratio relative to the AAV9 wild-type control.

To investigate tropism in marmoset monkeys, AAV particles comprising the TTM-001 capsid variant, the TTM-002 capsid variant, an AAV9 capsid variant comprising SEQ ID NO: 966, or an AAV9 control, were intravenously injected into NHPs (n=2, >10 months of age) at a dose of 2E13 vg/kg (8.75E12 vg/mL). After 28-days in life, the brains and tissues (liver quadriceps, and heart) of the NHPs were collected and RNA was extracted. Following RNA recovery and RT-PCR amplification, a systematic NGS enrichment analysis was performed to calculate the fold enrichment ratio relative to the AAV9 wild-type control.

As provided in Table 63 (African green monkeys) and Table 64 (marmosets), both the TTM-001 and TTM-002 capsid variants demonstrated increased CNS tropism in diverse primate species. The TTM-001 capsid variant demonstrated a 73.6-fold increase in expression relative to AAV9 in the brain of cynomolgus macaques (Table 9, Example 10), a 43.5-fold increase in expression relative to AAV9 in the brain of African green monkeys, and a 703.3-fold increase in expression relative to AAV9 in the brain of marmosets. The TTM-002 capsid variant demonstrated a 62.6-fold increase in expression relative to AAV9 in the brain of cynomolgus macaques (Table 9), a 13.8-fold increase in expression relative to AAV9 in the brain of African green monkeys, and a 366.6-fold increase in expression relative to AAV9 in the brain of marmosets. Both TTM-001 and TTM-002 led to a significant increase in expression relative to AAV9 in the heart of both African green monkeys and marmosets (Table 63 and Table 64). The AAV9 capsid variant comprising SEQ ID NO: 963 also demonstrated in increase in expression relative to AAV9 in the brain and heart of both African green monkeys and marmosets. Furthermore, TTM-001, TTM-002, and the AAV9 capsid variant comprising SEQ ID NO: 966, also all led to increased expression in the brain of both BALB/c and C57Bl/6 mice (Table 11, Example 10), demonstrating an average fold change in expression relative to AAV9 across both species of mice of 63.1, 66.8, and 126.97, respectively.

TABLE 63
NGS-Fold Enrichment of TTM-001 (comprises SEQ ID NO: 941),
TTM-002 (comprises SEQ ID NO: 2), and an AAV9 capsid variant
comprising SEQ ID NO: 966 in African green monkeys

| | SEQ ID | Fold Enrichment relative to AAV9 | | | | | |
|---|---|---|---|---|---|---|---|
| Sequence | NO: | Brain | DRG | Heart | Liver DNA | Liver RNA | Muscle |
| SPHSKA | 941 | 43.525 | 1.010 | 184.789 | 0.242 | 1.547 | 1.715 |
| HDSPHK | 2 | 13.779 | 0.678 | 35.991 | 0.084 | 0.087 | 0.144 |
| SPHKYG | 966 | 9.805 | 0.071 | 44.865 | 0.085 | 0.136 | 0.234 |

TABLE 64
NGS-Fold Enrichment of TTM-001 (comprises SEQ ID NO: 941),
TTM-002 (comprises SEQ ID NO: 2), and an AAV9 capsid variant
comprising SEQ ID NO: 966 in Marmosets

| | SEQ ID | Fold Enrichment relative to AAV9 | | | | |
|---|---|---|---|---|---|---|
| Sequence | NO: | Brain | Heart | Liver DNA | Liver RNA | Muscle |
| SPHSKA | 941 | 703.610 | 48.979 | 0.268 | 0.779 | 0.425 |
| HDSPHK | 2 | 366.625 | 18.572 | 0.075 | 0.276 | 0.229 |
| SPHKYG | 966 | 150.209 | 17.232 | 0.045 | 0.014 | 0.146 |

Taken together, these data demonstrate that the AAV9 capsid variants of TTM-001 and TTM-002 demonstrated increased CNS tropism relative to the AAV9 control in the CNS across three diverse primate species and two species of mice, providing evidence of strong cross-species capacity. The AAV9 capsid variant comprising the amino acid sequence of SEQ ID NO: 966 also demonstrated strong CNS expression relative to the AAV9 control in two species of NHPs and two species of mice, also showing strong cross-species capacity.

### Example 14. Advanced Maturation of TTM-002 Capsid Variant in Mice

This Example describes additional maturation of the TTM-002 (SEQ ID NO: 982 (amino acid) and 984 (DNA), comprising SEQ ID NO: 2) capsid variant in mice. In order to mature the TTM-002 capsid variant, sets of three contiguous amino acids were randomized across the mutagenesis region in TTM-002 sequence, which spanned from position 450 to position 466, numbered according to SEQ ID NO: 982. Unlike the maturation performed in in Example 12, where the SPH motif that was observed in the AAV capsid variants that demonstrated the greatest fold-enrichment in the NHP brain relative wild-type AAV9 was not disrupted, in the maturation approach used in this Example, the SPH motif was not held constant to further explore the role of this motif in the capsid variant. The matured TTM-002 capsid variants that resulted from the maturation approach were pooled together for subsequent testing and characterization in mice.

The library of matured AAV capsid variants generated from the TTM-002 matured AAV capsid variant were intravenously injected into the tail vein of three CD-1 Outbred mice (Charles River; 6-8 weeks of age) at a dose of 1.0×10¹² VG/dose. After about 28 days in life, the brains of the mice were isolated and RNA was extracted. Following RNA recovery and RT-PCR amplification, a systematic NGS enrichment analysis was performed to calculate the fold enrichment ratio relative to the corresponding TTM-002 non-matured control, and the peptides comprised within the variants were identified. Variants were filtered by those with a raw virus count in the sample above 10 and a coefficient of variance (CV) that was greater than 1 (identifies the peptides/variants reliably detected in the majority of the samples isolated from the three mice).

Following the advanced maturation screen and filtering of the variants, 1302 variants demonstrated an increase in expression relative to the non-matured TTM-002 capsid variant in the brain of the outbred mice. Of the 1302 variants with improved tropism relative to the non-matured TTM-002, 1283 comprised the SPH motif in the same position as the non-matured TTM-002 capsid variant (e.g., immediately subsequent to position 455, relative to a reference sequence numbered according to the amino acid sequence of SEQ ID NO: 138 or 982). Mutations in the region of the SPH motif present in the non-matured TTM-002 capsid variant only consistently appear in those variants with a fold change of 0.2 or 0.1 or lower relative to the non-matured TTM-002 control in the brain of the mice. This indicates that the SPH motif may be important to the increased brain tropism that observed for the TTM-002 capsid variant. In instances when the SPH motif was disrupted, the fold change of the matured variants of TTM-002 decreased considerably in relation to the non-matured TTM-002 variant which comprised the SPH motif.

### Example 15. Tropism of TTM-002 AAV Capsid Variant

This Example further investigates the tropism and CNS cells transduced by the TTM-002 capsid variant (SEQ ID NO: 982 (amino acid) and 984 (DNA), comprising SEQ ID NO: 2), as outlined in Table 3 above. The amino acid and DNA sequences of TTM-002 are provided, e.g., in Tables 4 and 5, respectively.

AAV particles were generated with the TTM-002 capsid variant encapsulating a GFP transgene (AAV_TTM-002.GFP) or the HER-53 genetic element (SEQ ID NO: 5168) (AAV_TTM-002.HER-53).

Two tandem single cell RNA sequencing runs (scRNA-Seq) of mouse cells derived from the midbrain area were performed. In the first run, cells were pooled from two mice at day 28 post treatment with AAV_TTM-002.HER-53 particles. In the second run we treated with AAV_TTM-002.GFP particles, in the same manner but without xenografts. Orthotopic xenografts of MDA-MB-361-Luc #1 high passage cells grown as tumorspheres (in tumorsphere media; Sigma #C-28070) were injected (250,000 cells/2 μL/mouse) intracranially into 2-month old female SCID CB17 (Mutation: Icr-Prkdcscid/IcrIcoCrl) congenic immunodeficient mice (Charles River Laboratories). The injections were 2.5 mm (lateral), −1 mm (posterior) with respect to bregma, lowered −3 mm ventral and raised +0.5 mm dorsal to a final −2.5 mm ventral position. Two days later, dilutions of the AAV_TTM-002.HER-53 particles (run 1), or in the case without xenografts, dilutions of AAV_TTM-002.GFP particles (run 2) were prepared. IV injections of 100 μL (2.5e11 VG/animal) of the AAV_TTM-002.HER-53 particles or AAV_TTM-002.GFP particles were administered through the tail veins of mice (n=5 mice per groups). At 7 days post-injection, mice from run 1 were imaged in an AmiHTX (Spectral Imager) for bioluminescence of the human tumor cells due to expression of luciferase in response to intraperitoneal luciferin injections.

At 28 days post-injection with the AAV_TTM-002.HER-53 particles or AAV_TTM-002.GFP particles, two mice from each run were necropsied, brain samples were isolated, and the midbrain was dissected and isolated. The midbrain samples were then exposed to a cold protease inhibitor (Creative Biomart #NATE-0633) and were dissociated at 6 degrees centigrade. For the samples collected from the mice of run 1 (AAV_TTM-002.Payload particles), myelin depletion was performed (Miltenyi, #130-096-731), cells were filtered through a 40 μM mesh to filter out neurons) and loaded on a 10× chromium G chip. scRNA-Seq was performed (10× Genomics) and samples were sequenced on a NextGen500 Sequencing machine (Illumina). For the samples collected from run 2 (AAV_TTM-002.GFP particles and no xenografts), the cells were not myelin depleted or filtered through 40 μM mesh to include neurons. The cells isolated after run 2 were FACS sorted for GFP+/7AAD-(live GFP+ cells). The resultant cells were loaded on a 10× chromium G chip and the scRNA-Seq was run and processed (10× Genomics).

For run 1, the scRNA-Seq data was filtered to include cells with only greater than 1000 genes per cell and less than 5000, and less than 20 percent mitochondrial gene expression. For run 2, the scRNA-Seq data was filtered to include cells with only greater than 200 genes per cell and less than 5000, and less than 20 percent mitochondrial gene expression. The data were normalized, scaled, and integrated into one combined dataset. Clusters were generated with a resolution of 0.3 and each cluster identity was determined using a panel of cell type specific genes (e.g., as described in Brown et al., 2021. “Deep Parallel Characterization of AAV Tropism and AAV-Mediated Transcriptional Changes via Single-Cell RNA Sequencing”. Front. Immunol. 12:730825; the contents of which are hereby incorporated by reference in its entirety). The percentage of GFP sorted cells per cluster was calculated as was the percentage of payload expressing genes per cluster as parallel measures of TTM-002 transduction.

For HER-53 expressing cells, endothelial cells had the highest proportion of HER-53 positive cells, followed by astrocytes (Table 65). For GFP+ sorted cells, endothelial cells had the highest proportion of GFP positive cells, and astrocytes were the third highest cell type when sorting by proportion of cells expressing GFP (Table 65). These data indicate TTM-002 transduction exhibits an endothelial and astrocytic tropism. Furthermore, the astrocytic cluster had the second highest level of expression of Olig2 (oligodendrocytes demonstrated the greatest Olig2 expression). IHC staining was performed on brain samples isolated from AAV_TTM-002.GFP infected mice and demonstrated that GFP co-localized with some but not all Olig2+ cells. No co-staining was observed with mylein basic protein (MBP), a marker of oligodendrocytes. Co-staining with GFP was also not observed in NeuN positive cells (neurons), GFAP positive cells (astrocytes), and Iba1 positive cells (microglia). GFP staining was observed throughout the sagittal section of the mouse brain, which was demonstrative of increased staining in the midbrain. The GFP expressing cells observed did not have a bipolar morphology like oligodendrocyte progenitor (OPC) cells and therefore, together with the scRNA-Seq data, these results indicated that at day 28 post AAV treatment, Olig2+ astrocytes in the midbrain are being transduced by AAV particles comprising a TTM-002 capsid, in a cell type specific tropism.

TABLE 65
Quantification of HER-53 positive cells and GFP positive cells

| Cluster | % HER-53 | Cluster | % GFP Cells/ |
|---|---|---|---|
| Identity | Cells/Cluster | Identity | Cluster |
| Endothelial-2 | 6.58 | Endothelial-2 | 6.58 |
| Astrocyte | 4.50 | Endothelial-1 | 3.45 |
| Pericytes | 4.23 | Ventrolateral and | 2.38 |
| | | leptomeningeal | |
| | | cells (VLM) | |
| Mature Oligos | 3.85 | Astrocyte | 2.37 |
| Endothelial-1 | 3.09 | Vascular | 1.03 |
| | | smooth muscle | |
| | | cells (VSC) | |
| Committed Oligos | 1.90 | Pericytes | 0.77 |
| Vascular | 1.72 | Microglia | 0.00 |
| smooth muscle | | | |
| cells (VSC) | | | |
| Microglia | 0.40 | Committed Oligos | 0.00 |
| Macrophages | 0.00 | Macrophages | 0.00 |
| Ventrolateral and | 0.00 | Oligodendrocytes | 0.00 |
| leptomeningeal | | | |
| cells (VLM) | | | |
| Oligodendrocytes | 0.00 | Committed Oligos-2 | 0.00 |
| Committed Oligos-2 | 0.00 | Mature Oligos | 0.00 |

### Example 16. Prophylactic Treatment of HER-75 Vectorized in TTM-002 Capsid Variant

This example investigated prophylactic treatment in mice following intravenous administration of AAV particles comprising the TTM-002 capsid variant (SEQ ID NO: 982 (amino acid) and 984 (DNA), comprising SEQ ID NO: 2) encapsulating a HER-75 genetic element (SEQ ID NO: 5190, with an Fc region comprising the following substitutions L235V, F243L, R292P, Y300L, and P396L, numbered according to the EU index as in Kabat) (AAV_TTM-002.HER-75) as compared to treatment with AAV particles comprising the TTM-002 capsid variant encapsulating a genetic element encoding an IgG1 control with the modified Fc region of HER-75 (comprising the following substitutions L235V, F243L, R292P, Y300L, and P396L, numbered according to the EU index as in Kabat) (AAV_TTM-002.Control). Dilutions of the AAV_TTM-002.HER-75 or AAV_TTM-002. Control particles were prepared and intravenous injections of 100 μL (2.5e11 VG/animal) were administered through the tail veins of female mice, (n=5 mice per cohort; Fox Chase SCID CB17 Mutation: Icr-Prkdcscid/IcrIcoCrl Congenic Immunodeficient from Charles River Laboratories (#3611); 75 days old). Ten days post-administration of the AAV particles, orthotopic xenografts of MDA-MB-361-Luc #1 passage #43 cells grown as tumorspheres (in tumorsphere media; Sigma #C-28070) were injected intracranially (250,000 cells/2 μL/mouse). The injections were 2.5 mm (lateral), −1 mm (posterior) with respect to bregma, lowered −3 mm ventral and raised +0.5 mm dorsal to a final −2.5 mm ventral position. Starting at day 7 post-injection of the xenograft until 48 days post-xenograft, mice were imaged weekly in an AmiHTX (Spectral Imager) for bioluminescence of the human tumor cells expression of luciferase in response to intraperitoneal luciferin injections.

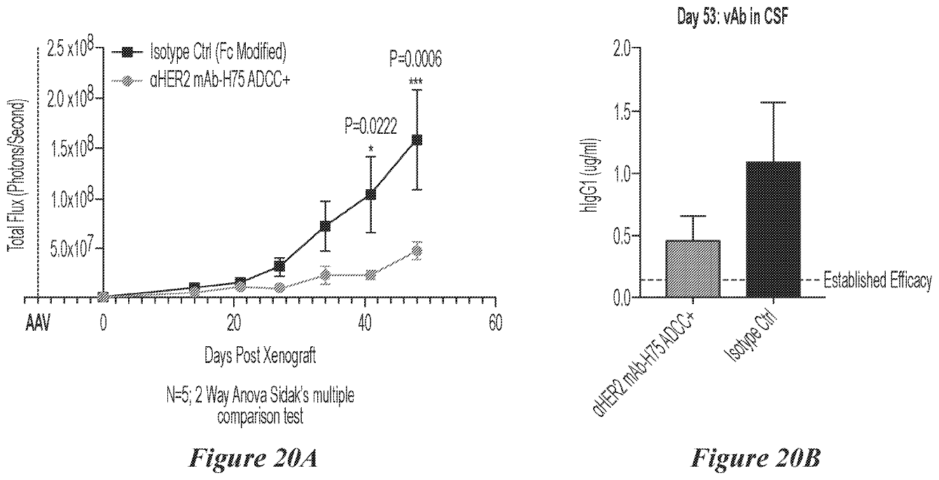

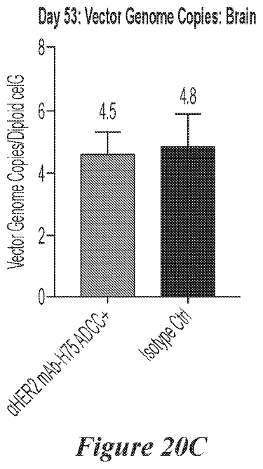

Prophylactic treatment 10 days prior to xenograft with IV administration of AAV_TTM-002.HER-75 (dosed at 2.5e11 VG/mouse) demonstrated persistent and significant attenuation of tumor growth as compared to isotype control (hIgG1) treated mice (FIG. 20A). FIG. 20B shows the comparison of the levels of antibodies produced following administration of the AAV_TTM-002 particles encapsulating the genetic element encoding an isotype control antibody to the AAV_TTM-002 particles encapsulating the genetic element encoding HER-75 in the in the CSF, as measured by alphaLISA. By alphaLISA, CSF levels were found to be above the minimum effective dose of −140 ng/ml as determined by the IC50 of HER-53 in MDA-MB-361 cell proliferation assay. FIG. 20C shows the comparison of viral genome copies/diploid cell produced following administration of the AAV_TTM-002 particles encapsulating the genetic element encoding an isotype control antibody to the AAV_TTM-002 particles encapsulating the genetic element encoding HER-75 in the brain. Viral genome levels in the brain were comparable in mice treated prophylactically with AAV particles comprising the TTM-002 capsid variant encoding the isotype control (4.8 VGs/diploid cell) and AAV particles comprising the TTM-002 capsid variant encoding the HER-75 genetic element (4.5 VGs/diploid cell) (FIG. 20C). Additionally, the mice treated prophylactically with the AAV particles comprising the TTM-002 capsid variant encoding the HER-75 genetic element had a median survival of 130 days, compared to a median survival of 102 days in the mice treated prophylactically with the AAV particles comprising the TTM-002 capsid variant encoding the isotype control. Thus, prophylactic treatment with AAV particles comprising the TTM-002 capsid variant encoding the HER-75 genetic element led to a 27.5% increase in survival.

### Example 17. Individual Capsid Characterization of TTM-002 in NHPs

This example describes the transduction level, tropism, ability to cross the blood brain barrier, and overall spatial distribution in the central nervous system (CNS) and peripheral tissues of the AAV capsid variant TTM-002 (SEQ ID NO: 982 (amino acid) and 984 (DNA), comprising SEQ ID NO: 2) and TTM-001 (SEQ ID NO: 981 (amino acid) and 983 (DNA), comprising SEQ ID NO: 941), relative to AAV9 following intravenous administration in African green monkeys (Chlorocebus sabaeus) and marmosets (Callithrix jacchus).

A. Evaluation of TTM-002 in African Green Monkeys (Chlorocebus sabaeus)

AAV particles were generated with the TTM-002 capsid variant or the AAV9 capsid control which comprised a self-complementary viral genome encoding an histone H2b protein with an HA tag driven by a ubiquitous CBA promoter. The AAV particles comprising the TTM-002 capsid variant or the AAV9 capsid control were administered to the African green monkeys (Chlorocebus sabaeus) (n=2) intravenously at a dose of 1e12 VG/kg or 1e13 VG/kg. The in-life period was 28 days and then various CNS and peripheral tissues were collected for measuring transgene mRNA (expression) by RT-qPCR and viral DNA (biodistribution) by ddPCR.

As shown in Table 70, the TTM-002 capsid variant resulted in increased brain biodistribution in all brain regions investigated as compared to AAV9 at both doses tested. The TTM-002 capsid variant also led to increased transgene expression in the brain relative to AAV9 at both doses tested (Table 71). In the spinal cord, the TTM-002 capsid variant distributed to the cervical spinal cord and the spinal cord ventral horn at a higher level relative to AAV9 (Table 70) and it mediated higher transgene expression than AAV9 in both the full spinal cord and the ventral horn (Table 71). The TTM-002 capsid variant exhibited lower biodistribution (Table 70) and transgene expression (Table 71) in the DRG relative to AAV9, indicating that TTM-002 capsid variant was detargeted in the DRG relative to AAV9. Similar expression and distribution were observed by immunohistochemistry performed on these CNS tissues.

Distribution and transgene expression was also measured in the peripheral tissues of the liver, heart, and quadriceps. In the liver, TTM-002 capsid variant exhibited lower biodistribution (Table 70) and transgene expression (Table 71) relative to AAV9, indicating that TTM-002 capsid variant was detargeted in the liver relative to AAV9. In the heart, the TTM-002 capsid variant exhibited comparable levels of biodistribution relative to AAV9 (Table 70), but increased transgene expression relative to AAV9 (Table 71). In the quadriceps, TTM-002 capsid variant exhibited lower biodistribution (Table 70) and lower transgene expression (Table 71), relative to AAV9. Similar expression and distribution were observed by immunohistochemistry performed on these peripheral tissues.

TABLE 70
Quantification of viral genome copies per diploid genome (biodistribution) by ddPCR
following intravenous administration of AAV particles comprising a TTM-002 capsid

| | 1e12 VG/kg | | | 1e13 VG/kg | | |
|---|---|---|---|---|---|---|
| | AAV9 | TTM-002 | | AAV9 | TTM-002 | |
| | (VG copies/ | (VG copies/ | TTM-002 | (VG copies/ | (VG copies/ | TTM-002 |
| | diploid | diploid | relative to | diploid | diploid | relative to |
| Tissue | genome) | genome) | AAV9 | genome) | genome) | AAV9 |
| Putamen | 0.03 | 0.37 | 12.3 | 0.26 | 2.4 | 9.2 |
| Caudate | 0.02 | 0.58 | 29 | 0.14 | 2.1 | 14.7 |
| Thalamus | 0.06 | 0.21 | 3.5 | 0.25 | 1.0 | 4 |
| Hippocampus | 0.03 | 0.29 | 9.7 | 0.16 | 1.56 | 9.8 |
| Substantia | 0.05 | 0.34 | 6.8 | 0.37 | 1.38 | 3.7 |
| Nigra | | | | | | |
| Motor Cortex | 0.03 | 0.56 | 19 | 0.27 | 2.4 | 8.9 |
| Frontal Cortex | 0.04 | 0.67 | 17 | 0.20 | 3.6 | 18 |
| Temporal | 0.03 | 0.31 | 10 | 0.11 | 2.67 | 24 |
| Cortex | | | | | | |
| Cerebral | 0.008 | 0.08 | 10 | 0.03 | 0.16 | 5.3 |
| Cortex | | | | | | |
| Dentate | 0.06 | 0.10 | 1.7 | 0.32 | 3.21 | 10 |
| Nucleus | | | | | | |
| Cervical | 0.03 | 0.12 | 4 | 0.19 | 0.91 | 4.8 |
| Spinal Cord | | | | | | |
| Thoracic | 0.04 | 0.03 | 0.75 | 0.36 | 0.38 | 1.1 |
| Spinal Cord | | | | | | |
| Lumbar Spinal | 0.04 | 0.03 | 0.75 | 0.29 | 0.37 | 1.3 |
| Cord | | | | | | |
| C5 Ventral | 0.04 | 0.25 | 6.3 | 0.29 | 2.2 | 7.6 |
| Horn | | | | | | |
| L5 Ventral | 0.06 | 0.28 | 4.7 | 0.31 | 1.9 | 6.1 |
| Horn | | | | | | |
| Cervical DRG | 0.07 | 0.01 | −7 | 0.81 | 0.36 | −2.3 |
| Thoracic DRG | 0.06 | 0.01 | −6 | 1.31 | 0.43 | −3 |
| Lumbar DRG | 0.07 | 0.01 | −7 | 1.31 | 0.57 | −2.3 |
| Liver | 9.5 | 1.2 | −7.9 | 127 | 7.7 | −16.5 |
| Heart | 0.6 | 0.7 | 1.2 | 5.4 | 5.4 | 1 |
| Quadriceps | 0.2 | 0.06 | −3.3 | 1.7 | 0.6 | −2.8 |

TABLE 71
Quantification of transgene mRNA by RT-qPCR following intravenous
administration of AAV particles comprising a TTM-002 capsid

| | 1e12 VG/kg | | | 1e13 VG/kg | | |
|---|---|---|---|---|---|---|
| | AAV9 | TTM-002 | | AAV9 | TTM-002 | |
| | (transgene | (transgene | | (transgene | (transgene | |
| | mRNA fold | mRNA fold | | mRNA fold | mRNA fold | TTM- |
| | over | over | TTM-002 | over | over | 002 |
| | housekeeping | housekeeping | relative to | housekeeping | housekeeping | relative |
| Tissue | gene) (2^{-dCT}) | gene) (2^{-dCT}) | AAV9 | gene) (2^{-dCT}) | gene) (2^{-dCT}) | to AAV9 |
| Putamen | 0.02 | 0.3 | 15 | 0.09 | 4.22 | 47 |
| Caudate | 0.02 | 0.8 | 40 | 0.11 | 4.29 | 39 |
| Thalamus | 0.04 | 0.4 | 10 | 0.4 | 5.8 | 14.5 |
| Hippocampus | 0.02 | 0.4 | 20 | 0.1 | 4.3 | 43 |
| Substantia | 0.1 | 1.2 | 12 | 0.3 | 11.6 | 39 |
| Nigra | | | | | | |
| Motor Cortex | 0.08 | 5.00 | 63 | 0.36 | 21.8 | 61 |
| Frontal | 0.04 | 3.1 | 78 | 0.3 | 27.7 | 92 |
| Cortex | | | | | | |
| Temporal | 0.02 | 0.8 | 40 | 0.1 | 26.9 | 27 |
| Cortex | | | | | | |
| Cerebral | 0.04 | 1.1 | 28 | 0.2 | 17.4 | 87 |
| Cortex | | | | | | |
| Dentate | 0.3 | 0.9 | 3 | 1.8 | 42.0 | 23 |
| Nucleus | | | | | | |
| Cervical | 0.2 | 2.0 | 10 | 0.8 | 20.2 | 25 |
| Spinal Cord | | | | | | |
| Thoracic | 0.13 | 0.25 | 1.9 | 0.7 | 4.8 | 6.9 |
| Spinal Cord | | | | | | |
| Lumbar | 0.4 | 0.5 | 1.3 | 2.2 | 9.2 | 4.2 |
| Spinal Cord | | | | | | |
| C5 Ventral | 0.2 | 1.4 | 7 | 1.7 | 33 | 19 |
| Horn | | | | | | |
| L5 Ventral | 1.1 | 3.4 | 3.1 | 12.4 | 102 | 8.2 |
| Horn | | | | | | |
| Cervical | 3.6 | 1.2 | −3 | 63.1 | 15.9 | −4 |
| DRG | | | | | | |
| Thoracic | 1.8 | 1.3 | −1.4 | 43.9 | 15.7 | −2.8 |
| DRG | | | | | | |
| Lumbar DRG | 1.9 | 1.0 | −1.9 | 34.9 | 27.6 | −1.3 |
| Liver | 0.88 | 0.25 | −3.5 | 2.2 | 0.97 | −2.3 |
| Heart | 8.7 | 42 | 4.8 | 110 | 363 | 3.3 |
| Quadriceps | 9.7 | 1.1 | −8.3 | 59 | 21 | −2.8 |

Taken together, these data demonstrate that TTM-002 is an enhanced CNS tropic capsid in NHPs (African green monkeys) that can infect non-neuronal cells. TTM-002 was also detargeted in the DRG and liver relative to AAV9, but showed increased transgene expression in the heart relative to AAV9. Additionally, the TTM-002 capsid variant was able to successfully penetrate the blood brain barrier following intravenous injection.

B. Evaluation of TTM-001 and TTM-002 in Marmosets (Callithrix jacchus)

AAV particles were generated with the TTM-002 capsid variant, the TTM-001 capsid variant, or the AAV9 capsid control which comprised a self-complementary viral genome encoding a histone H2b protein with an MYC tag (TTM-002 capsid variant), His tag (TTM-001 capsid variant), or HA tag (AAV9 control capsid) driven by a ubiquitous CAG promoter. The AAV particles comprising the TTM-002 capsid variant, the TTM-001 capsid variant, or the AAV9 capsid control were administered to the marmosets (Callithrix jacchus) (n=3) intravenously in a single solution, at the doses indicated in Table 29. The in-life period was 28 days and then various CNS and peripheral tissues were collected for measuring transgene mRNA (expression) by RT-qPCR, protein expression by IHC, and viral DNA (biodistribution) by ddPCR. Data were then normalized to the dose of each viral vector in the dosing solution.

TABLE 29
Titer of the AAV particles comprising the various
capsids in solution dosed in marmosets

| Capsid Variant | Actual Titer Dosed | Ratio of Capsid Variant to AAV9 |
|---|---|---|
| TTM-001 | 1.44 vg/mL | 0.34 |
| AAV9 | 4.00 vg/mL | 1.0 |
| TTM-002 | 4.17 vg/mL | 1.0 |

As shown in Table 72, both the TTM-001 and TTM-002 capsid variants demonstrated increased biodistribution in the caudate and motor cortex in the brain of the marmosets relative to the AAV9 control. The TTM-001 and TTM-002 capsid variants also led to increased transgene expression (Table 73) in the caudate and motor cortex in the brain of the marmosets. In fact, biodistribution and transgene expression were increased over 100-400 fold for both TTM-001 and TTM-002 in the brain relative to AAV9. Similar expression and distribution was observed by immunohistochemistry. More specifically, staining for TTM-001 and TTM-002 was detected in the mid-brain, caudate, putamen, thalamus, and cerebellum, and this staining was increased for both capsid variants in each of these brain tissues relative to AAV9. Staining for TTM-001 and TTM-002 was also observed in the molecular and granule layer of the cerebellum.

Distribution and transgene expression was also measured in the peripheral tissues of the liver, heart, and quadriceps. In the liver, the TTM-002 capsid variant exhibited lower biodistribution (Table 72) and transgene expression (Table 72) relative to AAV9, indicating that the TTM-002 capsid variant was detargeted in the liver relative to AAV9 in marmosets. The TTM-001 capsid variant demonstrated comparable biodistribution and transgene expression in the liver (Table 72 and Table 73) as well as comparable transgene expression in the heart and muscle (Table 73) relative to AAV9. Both TTM-001 and TTM-002 led to decreased biodistribution (Table 72) relative to AAV9 in the heart and muscle, and TTM-002 also resulted in lower transgene expression in the heart and muscle relative to AAV9 (Table 73).

TABLE 72
Quantification of viral genome copies per diploid genome (biodistribution) by
ddPCR following intravenous administration of AAV particles comprising a TTM-
001 capsid or a TTM-002 capsid normalized to the actual titer of the viral vector
in the dosing solution (vg/dg = viral genome copies/diploid genome)

| | Tissue | | | | | | | | | |
|---|---|---|---|---|---|---|---|---|---|---|
| | Caudate | | Motor Cortex | | Heart | | Muscle | | Liver | |
| | | vg/dg | | vg/dg | | vg/dg | | vg/dg | | vg/dg |
| | | relative | | relative | | relative | | relative | | relative |
| Capsid | vg/dg | to AAV9 | vg/dg | to AAV9 | vg/dg | to AAV9 | vg/dg | to AAV9 | vg/dg | to AAV9 |
| TTM-001 | 1.67 | 142.70 | 2.69 | 124.06 | 0.28 | 0.53 | 0.08 | 0.39 | 14.86 | 0.99 |
| TTM-002 | 3.55 | 294.36 | 5.80 | 264.86 | 0.33 | 0.69 | 0.08 | 0.32 | 6.92 | 0.49 |
| AAV9 | 0.01 | 1.00 | 0.02 | 1.00 | 0.48 | 1.00 | 0.23 | 1.00 | 13.79 | 1.00 |

TABLE 73
Quantification of transgene mRNA by RT-qPCR following intravenous administration of AAV
particles comprising a TTM-001 capsid or a TTM-002 capsid normalized to the actual titer
of the viral vector in the dosing solution (mRNA = transgene mRNA fold over housekeeping
gene; rel. to AAV9 = transgene mRNA fold over housekeeping gene relative to AAV9)

| | Tissue | | | | | | | | | |
|---|---|---|---|---|---|---|---|---|---|---|
| | Caudate | | Motor Cortex | | Heart | | Muscle | | Liver | |
| | | rel. to | | rel. to | | rel. to | | rel. to | | rel. to |
| Capsid | mRNA | AAV9 | mRNA | AAV9 | mRNA | AAV9 | mRNA | AAV9 | mRNA | AAV9 |
| TTM- | 17.56 | 594.71 | 27.80 | 586.23 | 16.05 | 1.40 | 0.26 | 1.19 | 3.23 | 1.93 |
| 001 | | | | | | | | | | |
| TTM- | 14.21 | 479.39 | 19.73 | 410.40 | 2.78 | 0.27 | 0.06 | 0.46 | 0.62 | 0.36 |
| 002 | | | | | | | | | | |
| AAV9 | 0.03 | 1.00 | 0.05 | 1.00 | 12.67 | 1.00 | 0.15 | 1.00 | 1.85 | 1.00 |

These data in marmosets for TTM-002 were similar to those observed in African green monkeys, further demonstrating cross-species compatibility of the TTM-002 capsid variant.

Taken together, these data demonstrate that TTM-001 and TTM-002 are enhanced CNS tropic capsid in marmosets. TTM-002 was also detargeted in the liver, heart, and muscle relative to AAV9 in marmosets, where TTM-001 demonstrated comparable biodistribution and/or transgene expression in the liver, heart, and muscle compared to AAV9. Additionally, the TTM-001 and TTM-002 capsid variants were able to successfully penetrate the blood brain barrier following intravenous injection.

### Example 18. In Vivo Characterization of Vectorized Anti-HER2 Monospecific Antibody Molecules Comprising FcRN Modifications

The biodistribution of HER-04 (SEQ ID NO: 5163; wild-type Fc), HER-47 (SEQ ID NO: 5185; comprising the FcRN modifications: I253A, H310A, H435A, numbered according to the EU index as in Kabat), and HER-42 (SEQ ID NO: 5166; comprising the FcRN modification H435Q, numbered according to the EU index as in Kabat) vectorized in a VOY101 capsid protein (SEQ ID NO: 1) was quantified in mice to determine if FcRN modifications were able reduce serum levels of vectorized antibodies compared to the HER-04 wild-type Fc control.

CB17/SCID mice (BALB/C background; n=5) were intravenously administered the vectorized HER-004, HER-47, and HER-42 constructs with the VOY101 capsid protein at a dose of 2.5e11 VG/100 uL/injection. At weeks 1, 2 and 3 post IV injection, blood was drawn to measure serum concentrations of hIgG1 (antibody levels in the serum). At 29 days post intravenous administration of the AAV particles, the mice were sacrificed and necropsied to isolate cerebral spinal fluid (CSF), the brain, heart, and liver tissues for hIgG1 quantification (antibody levels in the CNS and peripheral tissues).

### PK/PD Dynamics in the Serum, CSF, Brain, Heart and Liver Post Intravenous Administration of the Vectorized Anti-HER2 Antibodies Comprising FcRN Modifications

AlphaLISA was used to quantify the levels of hIgG1 in the serum samples isolated at weeks 1, 2, and 3 post-treatment, and the CSF, brain, heart and liver samples isolated at 29 days post-treatment. Table 76 shows the concentration of the HER-04, HER-47, and HER-42 vectorized antibodies in the serum of the mice at weeks 1, 2, and 3 post-treatment. Table 77 provides the concentration of the HER-04, HER-47, and HER-42 vectorized antibodies in the brain, heart, liver, and CSF of the mice at 29-days post-treatment.

TABLE 76
Serum levels of anti-HER2 antibodies at weeks 1, 2, and 3 post-treatment
with vectorized HER-04, HER-47 (I253A, H310A, H435A) or HER-42 (H435Q)

| | Average Serum hIgG1 Levels (μg/ml) | | | Standard Deviation | | |
|---|---|---|---|---|---|---|
| Antibody | Week 1 | Week 2 | Week 3 | Week 1 | Week 2 | Week 3 |
| HER-04 | 26.8 | 48.0 | 77.5 | 10.2 | 17.8 | 37.7 |
| (control; wild-type) | | | | | | |
| HER-47 | 1.2 | 2.5 | 2.5 | 0.8 | 1.9 | 2.8 |
| (I253A, H310A, H435A) | | | | | | |
| HER-42 | 38.2 | 67.7 | | 13.7 | 44.8 | |
| (H435Q) | | | | | | |

TABLE 77
Levels of anti-HER2 antibodies in the CSF, liver, heart,
and brain at 29 days post-treatment with vectorized
HER-04, HER-47 (I253A, H310A, H435A) or HER-42 (H435Q)

| | Average hIgG1 Levels (μg/ml) | | | | Standard Deviation | | | |
|---|---|---|---|---|---|---|---|---|
| Antibody | Brain | Heart | Liver | CSF | Brain | Heart | Liver | CSF |
| HER-04 | 1.7 | 1.5 | 2.9 | 0.3 | 0.60 | 0.76 | 1.54 | 0.06 |
| (control; wild-type Fc) | | | | | | | | |
| HER-47 | 1.0 | 0.0 | 0.1 | 0.4 | 0.23 | 0.04 | 0.14 | 0.43 |
| (I253A, H310A, H435A) | | | | | | | | |
| HER-42 | 3.8 | 1.8 | 0.7 | 1.4 | 3.07 | 0.92 | 0.37 | 0.35 |
| (H435Q) | | | | | | | | |

As shown in Tables 76 and 77, the serum levels of the vectorized HER-47 antibody comprising the three FcRN modifications, I253A, H310A, H435A, numbered according to the EU index as in Kabat, was markedly reduced compared to the vectorized HER-04 control with the wild-type Fc region. However, the level of the vectorized HER-47 antibody comprising the three FcRN modifications was only partially reduced in the brain, relative to the HER-04 control. These data indicated that the FcRN modifications of I253A, H310A, H435A, numbered according to the EU index as in Kabat, may be useful in reducing serum levels of vectorized anti-HER2 antibodies.

### Example 19. Prophylactic Treatment of HER-103 Vectorized in TTM-002 Capsid Variant

This example investigated prophylactic treatment in mice following intravenous administration of AAV particles comprising the TTM-002 capsid variant (SEQ ID NO: 982 (amino acid) and 984 (DNA), comprising SEQ ID NO: 2) encapsulating a HER-103 genetic element (SEQ ID NO: 4047, with a GFAP promoter and encoding an anti-HER2 antibody comprising an Fc region comprising the following substitutions L235V, F243L, R292P, Y300L, P396L, I253A, H310A, and H435A numbered according to the EU index as in Kabat) (AAV_TTM-002.HER-103) as compared to treatment with AAV particles comprising the TTM-002 capsid variant encapsulating a genetic element encoding an IgG1 control with the modified Fc region of HER-103 comprising the following substitutions L235V, F243L, R292P, Y300L, P396L, I253A, H310A, and H435A numbered according to the EU index as in Kabat). The Fc region of the anti-HER2 antibody encoded by the HER-103 genetic element comprises mutations that increase ADCC activity and other mutations that reduce binding to the FcRN receptor.

Dilutions of the AAV_TTM-002.HER-103 or AAV_TTM-002. Control particles were prepared and intravenous injections of 100 μL were administered through the tail veins of female mice, (n=5 mice per cohort; Fox Chase SCID CB17 Mutation: Icr-Prkdcscid/IcrIcoCrl Congenic Immunodeficient from Charles River Laboratories (#3611); 75 days old). Increasing doses of 5e12 VG/kg (1e11 VG), 1.25e13 VG/kg (1.25e13 VG), and 2.5e13 VG/kg (2.5e13 VG) were administered to the mice. Ten days post-administration of the AAV particles, orthotopic xenografts of MDA-MB-361-Luc #1 passage #43 cells grown as tumorspheres (in tumorsphere media; Sigma #C-28070) were injected intracranially (250,000 cells/2 μL/mouse). The injections were 2.5 mm (lateral), −1 mm (posterior) with respect to bregma, lowered −3 mm ventral and raised +0.5 mm dorsal to a final −2.5 mm ventral position. Starting at day 7 post-injection of the xenograft until 48 days post-xenograft, mice were imaged weekly in an AmiHTX (Spectral Imager) for bioluminescence of the human tumor cells expression of luciferase in response to intraperitoneal luciferin injections. Serum samples were also collected at day 28 post intravenous administration to measure serum antibody concentration.

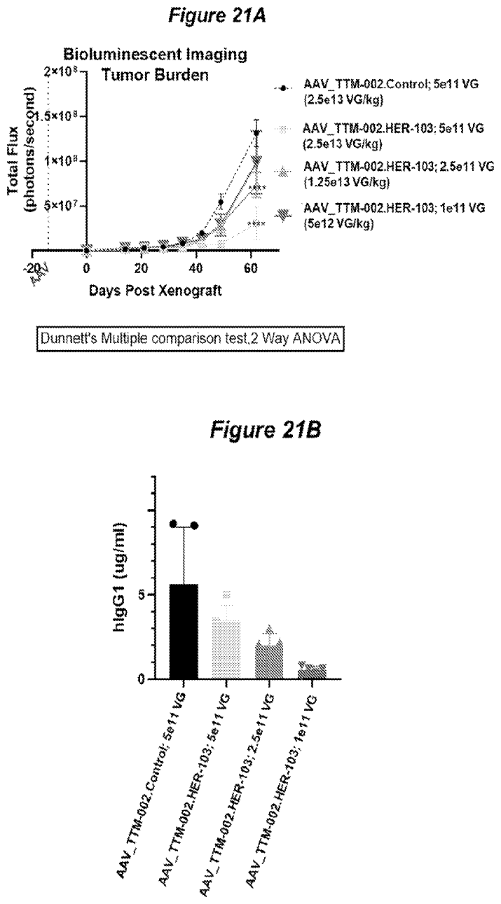

Prophylactic treatment 10 days prior to xenograft with IV administration of AAV_TTM-002.HER-103 (dosed at 2.5e11 VG/mouse) demonstrated persistent and significant abrogation of tumor growth as compared to isotype control (hIgG1) treated mice (FIG. 21A; 2 Way ANOVA, Dunnett's multiple comparison test). Additionally, there was a dose dependent response observed with respect to tumor growth attenuation across the doses investigated. FIG. 21B shows the comparison of the levels of antibodies produced following administration of the AAV_TTM-002 particles encapsulating the genetic element encoding an isotype control antibody to the AAV_TTM-002 particles encapsulating the genetic element encoding HER-103 in the in the serum, as measured by alphaLISA for the different doses investigated. A dose response was also observed with respect to serum antibody concertation (FIG. 21B), and levels were low. As shown in Table 78, antibody levels were reduced in the serum of mice treated with AAV_TTM-002.HER-103 encoding an antibody comprising mutations that reduced FcRN binding compared to mice treated with AAV particles comprising the TTM-002 capsid encoding the HER-75 genetic element comprising a constitutive CBA promoter bur not the Fc mutations that reduced FcRN binding (SEQ ID NO: 5190) (AAV_TTM-002.HER-75), or the HER-93 genetic element comprising a GFAP promoter but not the Fc mutations that reduce FcRN binding (SEQ ID NO: XX) (AAV_TTM-002.HER-93).

Antibody levels were also quantified in the brain by Alphalisa. Quantification of human IgG1 was performed and normalized to percent total protein in the brain (Table 78). In contrast to the serum, antibody levels in the brain of mice treated with AAV_TTM-002.HER-103, were higher and equivalent to antibody levels expressed in mice treated with AAV particles comprising the TTM-002 capsid encoding the HER-75 genetic element comprising a constitutive CBA promoter bur not the Fc mutations that reduced FcRN binding (SEQ ID NO: 5190) (AAV_TTM-002.HER-75) or the HER-93 genetic element comprising a GFAP promoter but not the Fc mutations that reduce FcRN binding (SEQ ID NO: XX) (AAV_TTM-002.HER-93).

Taken together, these data indicate that the mutations in the Fc region of the anti-HER2 antibody encoded by the HER-103 genetic element resulted in reduced serum levels, while still maintaining high antibody levels in the brain, and was able to attenuate tumor burden in a preclinical orthotopic human xenograft model of HER2+ breast cancer brain metastases in mice.

TABLE 78
Quantification of serum or brain antibody levels
following vectorized antibody delivery

| | Serum Human | Brain Human |
|---|---|---|
| | IgG Levels | IgG Levels |
| Treatment | (μg/mL) | (μg/mL) |
| AAV_TTM-002.HER-103 | 1.62 | 1.6 |
| AAV_TTM-002.HER-75 | 91.76 | 1.7 |
| AAV_TTM-002.HER-93 | 36.56 | 1.8 |

### VIII. EQUIVALENTS AND SCOPE

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. An AAV particle comprising an AAV capsid variant and a nucleic acid encoding an antibody molecule which binds to human HER2/neu, wherein the AAV capsid variant comprises an amino acid sequence having the following formula:
[N1]-[N2]-[N3], wherein:
(i) optionally [N1] comprises X1, X2, and X3, wherein at least one of X1, X2, or X3 is G;
(ii) [N2] comprises the amino acid sequence of SPH; and
(iii) [N3] comprises X4, X5, and X6, wherein at least one of X4, X5, or X6 is a basic amino acid, e.g., a K or R;
wherein [N1]-[N2]-[N3] is present from N-terminus to C-terminus, immediately subsequent to position 452, numbered according to any one of SEQ ID NO: 982 or 981; and
wherein the AAV capsid variant comprises an amino acid sequence at least 95% identical to the amino acid sequence of positions 203-742, e.g., a VP3, of any one of SEQ ID NO: 982 or 981.

2. The AAV particle of claim 1, wherein:
(a) position X4 of [N3] is: K, S, A, V, T, G, F, W, V, N, or R;
(b) position X5 of [N3] is: S, K, T, F, I, L, Y, H, M, or R; and/or
(c) position X6 of [N3] is: G, A, R, M, I, N, T, Y, D, P, V, L, E, W, N, Q, K, or S.

3. The AAV particle of claim 1 or 2, wherein:
(i) [N3] comprises KSG, SKA, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG; or
(ii) [N2]-[N3] comprises SPHSKA (SEQ ID NO: 941), SPHKSG (SEQ ID NO: 946), SPHARM (SEQ ID NO: 947), SPHVKS (SEQ ID NO: 948), SPHASR (SEQ ID NO: 949), SPHVKI (SEQ ID NO: 950), SPHKKN (SEQ ID NO: 954), SPHVRM (SEQ ID NO: 955), SPHRKA (SEQ ID NO: 956), SPHKFG (SEQ ID NO: 957), SPHKIG (SEQ ID NO: 958), SPHKLG (SEQ ID NO: 959), SPHKTS (SEQ ID NO: 963), SPHKTT (SEQ ID NO: 964), SPHKTY (SEQ ID NO: 965), SPHKYG (SEQ ID NO: 966), SPHSKD (SEQ ID NO: 967), SPHSKP (SEQ ID NO: 968), SPHTRG (SEQ ID NO: 972), SPHVRG (SEQ ID NO: 973), SPHKRG (SEQ ID NO: 974), SPHGAR (SEQ ID NO: 975), SPHKSA (SEQ ID NO: 977), SPHKSR (SEQ ID NO: 951), SPHSKL (SEQ ID NO: 960), SPHSRA (SEQ ID NO: 969), SPHSKR (SEQ ID NO: 978), SPHSLR (SEQ ID NO: 952), SPHSRG (SEQ ID NO: 961), SPHSSR (SEQ ID NO: 970), SPHFLR (SEQ ID NO: 979), SPHSKW (SEQ ID NO: 953), SPHSKS (SEQ ID NO: 962), SPHWKA (SEQ ID NO: 971), SPHVRR (SEQ ID NO: 980), SPHSKT (SEQ ID NO: 6647), SPHSKG (SEQ ID NO: 6648), SPHGKA (SEQ ID NO: 6649), SPHNKA (SEQ ID NO: 6650), SPHSKN (SEQ ID NO: 6651), SPHAKA (SEQ ID NO: 6652), SPHSKV (SEQ ID NO: 6653), SPHKTG (SEQ ID NO: 6654), SPHTKA (SEQ ID NO: 6655), SPHKSL (SEQ ID NO: 6656), SPHKSE (SEQ ID NO: 6657), SPHKSV (SEQ ID NO: 6658), SPHKSW (SEQ ID NO: 6659), SPHKSN (SEQ ID NO: 6660), SPHKHG (SEQ ID NO: 6661), SPHKSQ (SEQ ID NO: 6662), SPHKSK (SEQ ID NO: 6663), SPHKLW (SEQ ID NO: 6664), SPHWKG (SEQ ID NO: 6665), SPHKMG (SEQ ID NO: 6666), SPHKMA (SEQ ID NO: 6667), or SPHRSG (SEQ ID NO: 976).

4. The AAV particle of any one of claims 1-3, wherein:
(a) position X1 of [N1] is: G, V, R, D, E, M, T, 1, S, A, N, L, K, H, P, W, or C;
(b) position X2 of [N1] is: H, S, V, L, N, D, R, P, G, T, I, A, E, Y, M, or Q; and/or
(c) position X3 of [N1] is: D, G, C, L, E, Y, H, V, A, N, P, or S.

5. The AAV particle of any one of claims 1-4, wherein:
(i) [N1] comprises GHD, GSG, GQD, VSG, CSG, GRG, CSH, GQS, GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS;
(ii)
[N1]-[N2] comprises
(SEQ ID NO: 6700)
GSGSPH,
(SEQ ID NO: 6701)
GHDSPH,
(SEQ ID NO: 6702)
GQDSPH,
(SEQ ID NO: 6703)
VSGSPH,
(SEQ ID NO: 6704)
CSGSPH,
(SEQ ID NO: 6705)
GRGSPH,
(SEQ ID NO: 6706)
CSHSPH,
(SEQ ID NO: 6707)
GQSSPH,
(SEQ ID NO: 6708)
GSHSPH,
(SEQ ID NO: 6709)
GDDSPH,
(SEQ ID NO: 6710)
GHESPH,
(SEQ ID NO: 6711)
GNYSPH,
(SEQ ID NO: 6712)
RVGSPH,
(SEQ ID NO: 6713)
GSCSPH,
(SEQ ID NO: 6714)
GLLSPH,
(SEQ ID NO: 6715)
MSGSPH,
(SEQ ID NO: 6716)
RNGSPH,
(SEQ ID NO: 6717)
TSGSPH,
(SEQ ID NO: 6718)
ISGSPH,
(SEQ ID NO: 6719)
GPGSPH,
(SEQ ID NO: 6720)
ESGSPH,
(SEQ ID NO: 6721)
SSGSPH,
(SEQ ID NO: 6722)
GNGSPH,
(SEQ ID NO: 6723)
ASGSPH,
(SEQ ID NO: 6724)
NSGSPH,
(SEQ ID NO: 6725)
LSGSPH,
(SEQ ID NO: 6727)
KSGSPH,
(SEQ ID NO: 6728)
HSGSPH,
(SEQ ID NO: 6729)
GTGSPH,
(SEQ ID NO: 6730)
PSGSPH,
(SEQ ID NO: 6731)
GSVSPH,
(SEQ ID NO: 6732)
RSGSPH,
(SEQ ID NO: 6733)
GIGSPH,
(SEQ ID NO: 6734)
WSGSPH,
(SEQ ID NO: 6735)
DSGSPH,
(SEQ ID NO: 6736)
IDGSPH,
(SEQ ID NO: 6737)
GLGSPH,
(SEQ ID NO: 6738)
DAGSPH,
(SEQ ID NO: 6739)
DGGSPH,
(SEQ ID NO: 6740)
MEGSPH,
(SEQ ID NO: 6741)
ENGSPH,
(SEQ ID NO: 6742)
GSASPH,
(SEQ ID NO: 6743)
KNGSPH,
(SEQ ID NO: 6744)
KEGSPH,
(SEQ ID NO: 6745)
AIGSPH,
(SEQ ID NO: 6746)
GYDSPH,
(SEQ ID NO: 6747)
GHGSPH,
(SEQ ID NO: 6748)
GRDSPH,
(SEQ ID NO: 6749)
GNDSPH,
(SEQ ID NO: 6750)
GPDSPH,
(SEQ ID NO: 6751)
GMGSPH,
(SEQ ID NO: 6752)
GQVSPH,
(SEQ ID NO: 6753)
GHNSPH,
(SEQ ID NO: 6754)
GHPSPH,
or
(SEQ ID NO: 6755)
GHSSPH;
or
(iii)
[N1]-[N2]-[N3] comprises
(SEQ ID NO: 6613)
GSGSPHSKA,
(SEQ ID NO: 6615)
GHDSPHKSG,
(SEQ ID NO: 6823)
GSGSPHARM,
(SEQ ID NO: 6824)
GSGSPHVKS,
(SEQ ID NO: 6825)
GQDSPHKSG,
(SEQ ID NO: 6826)
GSGSPHASR,
(SEQ ID NO: 6827)
GSGSPHVKI,
(SEQ ID NO: 6828)
GSGSPHKKN,
(SEQ ID NO: 6829)
GSGSPHVRM,
(SEQ ID NO: 6830)
VSGSPHSKA,
(SEQ ID NO: 6831)
CSGSPHSKA,
(SEQ ID NO: 6832)
GSGSPHRKA,
(SEQ ID NO: 6833)
CSGSPHKTS,
(SEQ ID NO: 6834)
CSHSPHKSG,
(SEQ ID NO: 6835)
GQSSPHRSG,
(SEQ ID NO: 6836)
GRGSPHASR,
(SEQ ID NO: 6837)
GRGSPHSKA,
(SEQ ID NO: 6838)
GSGSPHKFG,
(SEQ ID NO: 6839)
GSGSPHKIG,
(SEQ ID NO: 6840)
GSGSPHKLG,
(SEQ ID NO: 6841)
GSGSPHKTS,
(SEQ ID NO: 6842)
GSGSPHKTT,
(SEQ ID NO: 6843)
GSGSPHKTY,
(SEQ ID NO: 6844)
GSGSPHKYG,
(SEQ ID NO: 6845)
GSGSPHSKD,
(SEQ ID NO: 6846)
GSGSPHSKP,
(SEQ ID NO: 6847)
GSGSPHTRG,
(SEQ ID NO: 6848)
GSGSPHVRG,
(SEQ ID NO: 6849)
GSHSPHKRG,
(SEQ ID NO: 6850)
GSHSPHKSG,
(SEQ ID NO: 6851)
VSGSPHASR,
(SEQ ID NO: 6852)
VSGSPHGAR,
(SEQ ID NO: 6853)
VSGSPHKFG,
(SEQ ID NO: 6854)
GHDSPHKRG,
(SEQ ID NO: 6855)
GDDSPHKSG,
(SEQ ID NO: 6856)
GHESPHKSA,
(SEQ ID NO: 6857)
GHDSPHKSA,
(SEQ ID NO: 6858)
GNYSPHKIG,
(SEQ ID NO: 6859)
GHDSPHKSR,
(SEQ ID NO: 6860)
GSGSPHSKL,
(SEQ ID NO: 6861)
GSGSPHSRA,
(SEQ ID NO: 6862)
GSGSPHSKR,
(SEQ ID NO: 6863)
GSGSPHSLR,
(SEQ ID NO: 6864)
GSGSPHSRG,
(SEQ ID NO: 6865)
GSGSPHSSR,
(SEQ ID NO: 6866)
RVGSPHSKA,
(SEQ ID NO: 6867)
GSCSPHRKA,
(SEQ ID NO: 6868)
GSGSPHFLR,
(SEQ ID NO: 6869)
GSGSPHSKW,
(SEQ ID NO: 6870)
GSGSPHSKS,
(SEQ ID NO: 6871)
GLLSPHWKA,
(SEQ ID NO: 6872)
GSGSPHVRR,
(SEQ ID NO: 6873)
GSGSPHSKV,
(SEQ ID NO: 6874)
MSGSPHSKA,
(SEQ ID NO: 6875)
RNGSPHSKA,
(SEQ ID NO: 6876)
TSGSPHSKA,
(SEQ ID NO: 6877)
ISGSPHSKA,
(SEQ ID NO: 6878)
GPGSPHSKA,
(SEQ ID NO: 6879)
GSGSPHSKT,
(SEQ ID NO: 6880)
ESGSPHSKA,
(SEQ ID NO: 6881)
SSGSPHSKA,
(SEQ ID NO: 6882)
GNGSPHSKA,
(SEQ ID NO: 6883)
ASGSPHSKA,
(SEQ ID NO: 6884)
NSGSPHSKA,
(SEQ ID NO: 6885)
LSGSPHSKA,
(SEQ ID NO: 6886)
GGGSPHSKA,
(SEQ ID NO: 6887)
KSGSPHSKA,
(SEQ ID NO: 6888)
GGGSPHSKS,
(SEQ ID NO: 6889)
GSGSPHSKG,
(SEQ ID NO: 6890)
HSGSPHSKA,
(SEQ ID NO: 6891)
GTGSPHSKA,
(SEQ ID NO: 6892)
PSGSPHSKA,
(SEQ ID NO: 6893)
GSVSPHGKA,
(SEQ ID NO: 6894)
RSGSPHSKA,
(SEQ ID NO: 6895)
GSGSPHTKA,
(SEQ ID NO: 6896)
GIGSPHSKA,
(SEQ ID NO: 6897)
WSGSPHSKA,
(SEQ ID NO: 6898)
DSGSPHSKA,
(SEQ ID NO: 6899)
IDGSPHSKA,
(SEQ ID NO: 6900)
GSGSPHNKA,
(SEQ ID NO: 6901)
GLGSPHSKS,
(SEQ ID NO: 6902)
DAGSPHSKA,
(SEQ ID NO: 6903)
DGGSPHSKA,
(SEQ ID NO: 6904)
MEGSPHSKA,
(SEQ ID NO: 6905)
ENGSPHSKA,
(SEQ ID NO: 6906)
GSASPHSKA,
(SEQ ID NO: 6907)
GNGSPHSKS,
(SEQ ID NO: 6908)
KNGSPHSKA,
(SEQ ID NO: 6909)
KEGSPHSKA,
(SEQ ID NO: 6910)
AIGSPHSKA,
(SEQ ID NO: 6911)
GSGSPHSKN,
(SEQ ID NO: 6912)
GSGSPHAKA,
(SEQ ID NO: 6913)
GHDSPHKIG,
(SEQ ID NO: 6914)
GYDSPHKSG,
(SEQ ID NO: 6915)
GHESPHKSG,
(SEQ ID NO: 6916)
GHDSPHKTG,
(SEQ ID NO: 6917)
GRGSPHKRG,
(SEQ ID NO: 6825)
GQDSPHKSG,
(SEQ ID NO: 6918)
GHDSPHKSL,
(SEQ ID NO: 6919)
GHGSPHSKA,
(SEQ ID NO: 6920)
GHDSPHKSE,
(SEQ ID NO: 6830)
VSGSPHSKA,
(SEQ ID NO: 6921)
GRDSPHKSG,
(SEQ ID NO: 6922)
GNDSPHKSV,
(SEQ ID NO: 6923)
GQDSPHKIG,
(SEQ ID NO: 6924)
GHDSPHKSV,
(SEQ ID NO: 6925)
GPDSPHKIG,
(SEQ ID NO: 6926)
GPDSPHKSG,
(SEQ ID NO: 6927)
GHDSPHKSW,
(SEQ ID NO: 6928)
GHDSPHKSN,
(SEQ ID NO: 6929)
GMGSPHSKT,
(SEQ ID NO: 6930)
GHDSPHKHG,
(SEQ ID NO: 6931)
GQVSPHKSG,
(SEQ ID NO: 6932)
GDDSPHKSV,
(SEQ ID NO: 6933)
GHNSPHKSG,
(SEQ ID NO: 6934)
GNGSPHKRG,
(SEQ ID NO: 6935)
GHDSPHKYG,
(SEQ ID NO: 6936)
GHDSPHKSQ,
(SEQ ID NO: 6937)
GNDSPHKIG,
(SEQ ID NO: 6938)
GHDSPHKSK,
(SEQ ID NO: 6939)
GHDSPHKLW,
(SEQ ID NO: 6940)
GHPSPHWKG,
(SEQ ID NO: 6941)
GHDSPHKMG,
(SEQ ID NO: 6942)
GHDSPHKMA,
or
(SEQ ID NO: 6943)
GHSSPHRSG.

6. The AAV particle of any one of claims 1-5, wherein:
(a) [N1]-[N2]-[N3] comprises GHDSPHKSG (SEQ ID NO: 4698); or
(b) [N1]-[N2]-[N3] comprises GSGSPHSKA (SEQ ID NO: 4697).

7. The AAV particle of any one of claims 1-6, which further comprises:
(i) [N4], wherein [N4] comprises QNQQ (SEQ ID NO: 6945), WNQQ (SEQ ID NO: 6946), QYYV (SEQ ID NO: 6947), RRQQ (SEQ ID NO: 6948), GCGQ (SEQ ID NO: 6949), LRQQ (SEQ ID NO: 6950), RNQQ (SEQ ID NO: 6951), VNQQ (SEQ ID NO: 6952), FRLQ (SEQ ID NO: 6953), FNQQ (SEQ ID NO: 6954), LLQQ (SEQ ID NO: 6955), SNQQ (SEQ ID NO: 6956), RLQQ (SEQ ID NO: 6957), LNQQ (SEQ ID NO: 6958), QRKL (SEQ ID NO: 6959), LRRQ (SEQ ID NO: 6960), QRLR (SEQ ID NO: 6961), QRRL (SEQ ID NO: 6962), RRLQ (SEQ ID NO: 6963), RLRQ (SEQ ID NO: 6964), SKRQ (SEQ ID NO: 6965), QLYR (SEQ ID NO: 6966), QLTV (SEQ ID NO: 6967), QNKQ (SEQ ID NO: 6968), KNQQ (SEQ ID NO: 6969), QKQQ (SEQ ID NO: 6970), QTQQ (SEQ ID NO: 6971), QNHQ (SEQ ID NO: 6972), QHQQ (SEQ ID NO: 6973), QNQH (SEQ ID NO: 6974), QHRQ (SEQ ID NO: 6975), LTQQ (SEQ ID NO: 6976), QNQW (SEQ ID NO: 6977), QNTH (SEQ ID NO: 6978), RRRQ (SEQ ID NO: 6979), QYQQ (SEQ ID NO: 6980), QNDQ (SEQ ID NO: 6981), QNRH (SEQ ID NO: 6982), RDQQ (SEQ ID NO: 6983), PNLQ (SEQ ID NO: 6984), HVRQ (SEQ ID NO: 6985), PNQH (SEQ ID NO: 6986), HNQQ (SEQ ID NO: 6987), QSQQ (SEQ ID NO: 6988), QPAK (SEQ ID NO: 6989), QNLA (SEQ ID NO: 6990), QNQL (SEQ ID NO: 6991), QGQQ (SEQ ID NO: 6992), LNRQ (SEQ ID NO: 6993), QNPP (SEQ ID NO: 6994), QNLQ (SEQ ID NO: 6995), QDQE (SEQ ID NO: 6996), QDQQ (SEQ ID NO: 6997), HWQQ (SEQ ID NO: 6998), PNQQ (SEQ ID NO: 6999), PEQQ (SEQ ID NO: 7000), QRTM (SEQ ID NO: 7001), LHQH (SEQ ID NO: 7002), QHRI (SEQ ID NO: 7003), QYIH (SEQ ID NO: 7004), QKFE (SEQ ID NO: 7005), QFPS (SEQ ID NO: 7006), QNPL (SEQ ID NO: 7007), QAIK (SEQ ID NO: 7008), QNRQ (SEQ ID NO: 7009), QYQH (SEQ ID NO: 7010), QNPQ (SEQ ID NO: 7011), QHQL (SEQ ID NO: 7012), QSPP (SEQ ID NO: 7013), QAKL (SEQ ID NO: 7014), KSQQ (SEQ ID NO: 7015), QDRP (SEQ ID NO: 7016), QNLG (SEQ ID NO: 7017), QAFH (SEQ ID NO: 7018), QNAQ (SEQ ID NO: 7019), HNQL (SEQ ID NO: 7020), QKLN (SEQ ID NO: 7021), QNVQ (SEQ ID NO: 7022), QAQQ (SEQ ID NO: 7023), QTPP (SEQ ID NO: 7024), QPPA (SEQ ID NO: 7025), QERP (SEQ ID NO: 7026), QDLQ (SEQ ID NO: 7027), QAMH (SEQ ID NO: 7028), QHPS (SEQ ID NO: 7029), PGLQ (SEQ ID NO: 7030), QGIR (SEQ ID NO: 7031), QAPA (SEQ ID NO: 7032), QIPP (SEQ ID NO: 7033), QTQL (SEQ ID NO: 7034), QAPS (SEQ ID NO: 7035), QNTY (SEQ ID NO: 7036), QDKQ (SEQ ID NO: 7037), QNHL (SEQ ID NO: 7038), QIGM (SEQ ID NO: 7039), LNKQ (SEQ ID NO: 7040), PNQL (SEQ ID NO: 7041), QLQQ (SEQ ID NO: 7042), QRMS (SEQ ID NO: 7043), QGIL (SEQ ID NO: 7044), QDRQ (SEQ ID NO: 7045), RDWQ (SEQ ID NO: 7046), QERS (SEQ ID NO: 7047), QNYQ (SEQ ID NO: 7048), QRTC (SEQ ID NO: 7049), QIGH (SEQ ID NO: 7050), QGAI (SEQ ID NO: 7051), QVPP (SEQ ID NO: 7052), QVQQ (SEQ ID NO: 7053), LMRQ (SEQ ID NO: 7054), QYSV (SEQ ID NO: 7055), QAIT (SEQ ID NO: 7056), QKTL (SEQ ID NO: 7057), QLHH (SEQ ID NO: 7058), QNII (SEQ ID NO: 7059), QGHH (SEQ ID NO: 7060), QSKV (SEQ ID NO: 7061), QLPS (SEQ ID NO: 7062), IGKQ (SEQ ID NO: 7063), QAIH (SEQ ID NO: 7064), QHGL (SEQ ID NO: 7065), QFMC (SEQ ID NO: 7066), QNQM (SEQ ID NO: 7067), QHLQ (SEQ ID NO: 7068), QPAR (SEQ ID NO: 7069), QSLQ (SEQ ID NO: 7070), QSQL (SEQ ID NO: 7071), HSQQ (SEQ ID NO: 7072), QMPS (SEQ ID NO: 7073), QGSL (SEQ ID NO: 7074), QVPA (SEQ ID NO: 7075), HYQQ (SEQ ID NO: 7076), QVPS (SEQ ID NO: 7077), RGEQ (SEQ ID NO: 7078), PGQQ (SEQ ID NO: 7079), LEQQ (SEQ ID NO: 7080), QNQS (SEQ ID NO: 7081), QKVI (SEQ ID NO: 7082), QNND (SEQ ID NO: 7083), QSVH (SEQ ID NO: 7084), QPLG (SEQ ID NO: 7085), HNQE (SEQ ID NO: 7086), QIQQ (SEQ ID NO: 7087), QVRN (SEQ ID NO: 7088), PSNQ (SEQ ID NO: 7089), QVGH (SEQ ID NO: 7090), QRDI (SEQ ID NO: 7091), QMPN (SEQ ID NO: 7092), RGLQ (SEQ ID NO: 7093), PSLQ (SEQ ID NO: 7094), QRDQ (SEQ ID NO: 7095), QAKG (SEQ ID NO: 7096), QSAH (SEQ ID NO: 7097), QSTM (SEQ ID NO: 7098), QREM (SEQ ID NO: 7099), QYRA (SEQ ID NO: 7100), QRQQ (SEQ ID NO: 7101), QWQQ (SEQ ID NO: 7102), QRMN (SEQ ID NO: 7103), GDSQ (SEQ ID NO: 7104), QKIS (SEQ ID NO: 7105), PSMQ (SEQ ID NO: 7106), SPRQ (SEQ ID NO: 7107), MEQQ (SEQ ID NO: 7108), QYQN (SEQ ID NO: 7109), QIRQ (SEQ ID NO: 7110), QSVQ (SEQ ID NO: 7111), RSQQ (SEQ ID NO: 7112), QNKL (SEQ ID NO: 7113), QIQH (SEQ ID NO: 7114), PRQQ (SEQ ID NO: 7115), HTQQ (SEQ ID NO: 7116), QRQH (SEQ ID NO: 7117), RNQE (SEQ ID NO: 7118), QSKQ (SEQ ID NO: 7119), QNQP (SEQ ID NO: 7120), QSPQ (SEQ ID NO: 7121), QTRQ (SEQ ID NO: 7122), QNLH (SEQ ID NO: 7123), QNQE (SEQ ID NO: 7124), LNQP (SEQ ID NO: 7125), QNQD (SEQ ID NO: 7126), QNLL (SEQ ID NO: 7127), QLVI (SEQ ID NO: 7128), RTQE (SEQ ID NO: 7129), QTHQ (SEQ ID NO: 7130), QDQH (SEQ ID NO: 7131), QSQH (SEQ ID NO: 7132), VRQQ (SEQ ID NO: 7133), AWQQ (SEQ ID NO: 7134), QSVP (SEQ ID NO: 7135), QNIQ (SEQ ID NO: 7136), LDQQ (SEQ ID NO: 7137), PDQQ (SEQ ID NO: 7138), ESQQ (SEQ ID NO: 7139), QRQL (SEQ ID NO: 7140), QIIV (SEQ ID NO: 7141), QKQS (SEQ ID NO: 7142), QSHQ (SEQ ID NO: 7143), QFVV (SEQ ID NO: 7144), QSQP (SEQ ID NO: 7145), QNEQ (SEQ ID NO: 7146), INQQ (SEQ ID NO: 7147), RNRQ (SEQ ID NO: 7148), RDQK (SEQ ID NO: 7149), QWKR (SEQ ID NO: 7150), ENRQ (SEQ ID NO: 7151), QTQP (SEQ ID NO: 7152), QKQL (SEQ ID NO: 7153), RNQL (SEQ ID NO: 7154), ISIQ (SEQ ID NO: 7155), QTVC (SEQ ID NO: 7156), QQIM (SEQ ID NO: 7157), LNHQ (SEQ ID NO: 7158), QNQA (SEQ ID NO: 7159), QMIH (SEQ ID NO: 7160), RNHQ (SEQ ID NO: 7161), or QKMN (SEQ ID NO: 7162); and/or
(ii) [N0], wherein [N0] comprises TIN, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TII, TFP, TEK, EIN, TVN, TFN, SIN, TER, TSY, ELH, AIN, SVN, TDN, TFH, TVH, TEN, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAI, AEN, AET, ETA, NNL.

8. The AAV capsid particle of claim 7, wherein [N0]-[N1]-[N2]-[N3]-[N4] comprises the amino acid sequence of any one of SEQ ID NOs: 2243, 2242, 2242-2886.

9. The AAV particle of claim 7 or 8, which comprises from N-terminus to C-terminus [N0]-[N1]-[N2]-[N3]-[N4], wherein:
(A) (i) [N0] is present immediately subsequent to position 449 and replaces positions 450-452 (e.g., T450, I451, and N452), numbered according to the amino acid sequence of any one of SEQ ID NOs: 982, 981, or 138;
(ii) [N1] is present immediately subsequent to position 452 and replaces positions 453-455, numbered according to the amino acid sequence of SEQ ID NO: 138;
(iii) [N2] is present immediately subsequent to position 455, numbered according to the amino acid sequence of SEQ ID NO: 138;
(iv) [N3] is present immediately subsequent to [N2]; and/or
(v) [N4] is present immediately subsequent to position 455 and replaces positions 456-459 (e.g., Q456, N457, Q458, and Q459), numbered according to the amino acid sequence of SEQ ID NO: 138; or
(B) (i) [N2]-[N3]-[N4] corresponds to positions 456-465 of SEQ ID NO: 982 or 981;
(ii) [N1]-[N2]-[N3] corresponds to positions 453-461 of SEQ ID NO: 982 or 981; and/or
(iii) [N0]-[N1]-[N2]-[N3]-[N4] corresponds to positions 450-465 of SEQ ID NO: 982 or 981.

10. An AAV particle comprising an AAV capsid variant and a nucleic acid encoding an antibody molecule which binds to human HER2/neu, wherein the AAV capsid variant comprises:
(a) the amino acid sequence of any of the sequences provided in Tables 1, 2A, 2B, 9-11, 13-19;
(b) an amino acid sequence comprising at least 3, 4, 5, 6, 7, 8, 9, 10, 11, 12, 13, 14, 15, 16, or 17 consecutive amino acids from any one of the sequences provided in Tables 1, 2A, 2B, 9-11, 13-19; or
(c) an amino acid sequence comprising at least one, two, or three but no more than four different amino acids, relative to any one of the sequences provided in Tables 1, 2A, 2B, 9-11, 13-19; or
(d) an amino acid sequence comprising at least one, two, or three but no more than four modifications, e.g., substitutions (e.g., conservative substitutions), relative to the amino acid sequence of any one of the sequences provided in Tables 1, 2A, 2B, 9-11, 13-19.

11. The AAV particle of claim 10, wherein:
(i) the 3 consecutive amino acids comprise HDS;
(ii) the 4 consecutive amino acids comprise HDSP (SEQ ID NO: 4702);
(iii) the 5 consecutive amino acids comprise HDSPH (SEQ ID NO: 4703); and/or
(iv) the 6 consecutive amino acids comprise HDSPHK (SEQ ID NO: 2);
optionally wherein the amino acid sequence of (i)-(iv) is present immediately subsequent to position 453, numbered according to SEQ ID NO: 982 or 138.

12. The AAV particle of any one of claim 10, wherein:
(i) the 3 consecutive amino acids comprise SPH;
(ii) the 4 consecutive amino acids comprise SPHS (SEQ ID NO: 4700);
(iii) the 5 consecutive amino acids comprise SPHSK (SEQ ID NO: 4701); and/or
(iv) the 6 consecutive amino acids comprise SPHSKA (SEQ ID NO: 941)
optionally wherein the amino acid sequence of (i)-(iv) is present immediately subsequent to position 455, numbered according to SEQ ID NO: 982 or 138.

13. The AAV particle of any one of claims 10-12, wherein the amino acid sequence is present:
(i) in loop IV, optionally wherein loop IV comprises positions 449-460, numbered according to SEQ ID NO: 138;
(ii) immediately subsequent to position 448, 449, 450, 451, 452, 453, 454, or 455, relative to a reference sequence numbered according to the amino acid sequence of SEQ ID NO: 138;
(iii) immediately subsequent to position 453, relative to a reference sequence numbered according to the amino acid sequence of SEQ ID NO: 138 or 982; and/or
(iv) immediately subsequent to position 455, relative to a reference sequence numbered according to the amino acid sequence of SEQ ID NO: 138 or 981.

14. The AAV particle of any one of claims 1-15, comprising:
(i) the amino acid sequence of HDSPHK (SEQ ID NO: 2), wherein the amino acid sequence is present immediately subsequent to position 453, relative to a reference sequence numbered according to the amino acid sequence of SEQ ID NO: 138 or 982; or
(ii) the amino acid sequence of SPHSKA (SEQ ID NO: 941), wherein the amino acid sequence is present immediately subsequent to position 455, relative to a reference sequence numbered according to the amino acid sequence of SEQ ID NO: 138 or 981.

15. The AAV particle of any one of the preceding claims, wherein:
(i) the AAV capsid variant further comprises a modification, e.g., an insertion, substitution (e.g., conservative substitution), and/or deletion, in loop I, II, VI and/or VIII;
(ii) the AAV capsid variant further comprises a substitution at position K449, e.g., a K449R substitution, numbered according to SEQ ID NO: 138;
(iii) the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 138;
(iv) the AAV capsid variant comprises an amino acid sequence comprising at least one, two or three, but no more than 30, 20 or 10 different amino acids relative to the amino acid sequence of SEQ ID NO: 138;
(v) the AAV capsid variant comprises the amino acid sequence of positions 138-742, e.g., a VP2, of SEQ ID NO: 138, or a sequence with at least 95%, 96%, 97%, 98%, or 99% sequence identity thereto;
(vi) the AAV capsid variant comprises the amino acid sequence of SEQ ID NO: 138, or an amino acid sequence with at least 95%, 96%, 97%, 98%, or 99% sequence identity thereto;
(vii) the AAV capsid variant comprises an amino acid sequence encoded by the nucleotide sequence of SEQ ID NO: 137, or a sequence with at least 95%, 96%, 97%, 98%, or 99% sequence identity thereto; and/or
(viii) the nucleotide sequence encoding the capsid variant comprises the nucleotide sequence of SEQ ID NO: 137, or a sequence with at least 95%, 96%, 97%, 98%, or 99% sequence identity thereto.

16. The AAV particle of any one of the preceding claims, which comprises:
(i) a VP1 protein, a VP2 protein, a VP3 protein, or a combination thereof;
(ii) the amino acid sequence corresponding to positions 138-742, e.g., a VP2, of SEQ ID NO: 981 or 982, or a sequence with at least 95%, 96%, 97%, 98%, or 99% sequence identity thereto;
(iii) the amino acid sequence of any one of SEQ ID NO: 981 or 982 (e.g., a VP1), or an amino acid sequence with at least 95%, 96%, 97%, 98%, or 99% sequence identity thereto;
(iv) an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 981 or 982; and/or
(v) an amino acid sequence comprising at least one, two or three, but not more than 30, 20 or 10 different amino acids, relative to the amino acid sequence of SEQ ID NO: 981 or 982.

17. The AAV particle of any one of claims 1-11 or 13-16, wherein:
(i) the AAV capsid variant comprises the amino acid sequence of SEQ ID NO: 982, or an amino acid sequence with at least 95, 96, 97, 98, or 99% sequence identity thereto;
(ii) the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 982;
(iii) the AAV capsid variant comprises an amino acid sequence comprising at least one, two or three, but not more than 30, 20 or 10 different amino acids, relative to the amino acid sequence of SEQ ID NO: 982;
(iv) the AAV capsid variant comprises an amino acid sequence encoded by the nucleotide sequence of SEQ ID NO: 984, or a nucleotide sequence with at least 95, 96, 97, 98, or 99% sequence identity thereto;
(v) the nucleotide sequence encoding the AAV capsid variant comprises the nucleotide sequence of SEQ ID NO: 984, or a nucleotide sequence with at least 95, 96, 97, 98, or 99% sequence identity thereto, optionally wherein the nucleotide sequence is codon optimized.

18. The AAV particle of any one of claims 1-10 or 12-16, wherein:
(i) the AAV capsid variant comprises the amino acid sequence of SEQ ID NO: 981, or an amino acid sequence with at least 95%, 96%, 97%, 98%, or 99% sequence identity thereto;
(ii) the AAV capsid variant comprises an amino acid sequence comprising at least one, two or three modifications, e.g., substitutions (e.g., conservative substitutions), but not more than 30, 20 or 10 modifications, e.g., substitutions (e.g., conservative substitutions), relative to the amino acid sequence of SEQ ID NO: 981; and/or
(iii) the AAV capsid variant comprises an amino acid sequence comprising at least one, two or three, but not more than 30, 20 or 10 different amino acids, relative to the amino acid sequence of SEQ ID NO: 981;
(iv) the AAV capsid variant comprises an amino acid sequence encoded by the nucleotide sequence of SEQ ID NO: 983, or a nucleotide sequence with at least 95%, 96%, 97%, 98%, or 99% sequence identity thereto; and/or
(v) the nucleotide sequence encoding the AAV capsid variant comprises the nucleotide sequence of SEQ ID NO: 983, or a nucleotide sequence with at least 95%, 96%, 97%, 98%, or 99% sequence identity thereto, optionally wherein the nucleotide sequence is codon optimized.

19. The AAV particle of any one of claims 1-18, wherein the encoded antibody molecule binds domain I of HER2, domain II of HER2, domain III of HER2, and/or domain IV of HER2, optionally domain IV of HER2 or domain II HER2.

20. The AAV particle of any one of claims 1-19, wherein the encoded antibody molecule comprises:
(i) a heavy chain variable region comprising three HC CDR sequences of any of the HC CDR sequences of Table 3A-3C; and/or
(ii) a light chain variable region comprising three LC CDR sequences of any of the LC CDR sequences of Table 3A-3C;
optionally wherein one, two, three, four, five, or all of the HC CDR and LC CDR sequences comprise at least 1-4, e.g., at least one, two, three, or four, modifications, e.g., substitutions, relative to the HC CDR and LC CDR sequences provided in Tables 3A-3C.

21. The AAV particle of any one of claims 1-20, wherein the encoded antibody molecule comprises:
(a) the amino acid W at position 102, the amino acid F at position 107, the amino acid A position 108, and the amino acid L at position 109, relative to a reference sequence numbered according to the amino acid sequence of SEQ ID NO: 5001;
(b) the amino acid W at position 102, the amino acid F at position 107, and the amino acid A position 108, relative to a reference sequence numbered according to the amino acid sequence of SEQ ID NO: 5001;
(c) the amino acid W at position 102, the amino acid F at position 107, and the amino acid L at position 109, relative to a reference sequence numbered according to the amino acid sequence of SEQ ID NO: 5001;
(d) the amino acid W at position 102, the amino acid A position 108, and the amino acid L at position 109, relative to a reference sequence numbered according to the amino acid sequence of SEQ ID NO: 5001;
(e) the amino acid F at position 107, the amino acid A position 108, and the amino acid L at position 109, relative to a reference sequence numbered according to the amino acid sequence of SEQ ID NO: 5001.

22. The AAV particle of any one of claims 1-21, wherein the encoded antibody molecule comprises:
(i) a heavy chain variable region comprising a HC CDR1 amino acid sequence of SEQ ID NO: 5281, a HC CDR2 amino acid sequence of SEQ ID NO: 5282, and an HC CDR3 amino acid sequence of SEQ ID NO: 5283, 6510, 6515, 6520, 6525 or 6530; and/or a light chain variable region comprising an LC CDR1 amino acid sequence of SEQ ID NO: 5287, an LC CDR2 amino acid sequence of SEQ ID NO: 5288, and an LC CDR3 amino acid sequence of SEQ ID NO: 5289;
(ii) a heavy chain variable region comprising a HC CDR1 amino acid sequence of SEQ ID NO: 5284, a HC CDR2 amino acid sequence of SEQ ID NO: 5285, and an HC CDR3 amino acid sequence of SEQ ID NO: 5286; and/or a light chain variable region comprising an LC CDR1 amino acid sequence of SEQ ID NO: 5287, an LC CDR2 amino acid sequence of SEQ ID NO: 5291, and an LC CDR3 amino acid sequence of SEQ ID NO: 5292;
(iii) a HC CDR1, a HC CDR2, a HC CDR3, a LC CDR1, a LC CDR2 and an LC CDR3 comprising the amino acid sequences of SEQ ID NO: 5281, 5282, 6510, 5287, 5288, and 5289, respectively;
(iv) a HC CDR1, a HC CDR2, a HC CDR3, a LC CDR1, a LC CDR2 and an LC CDR3 comprising the amino acid sequences of SEQ ID NO: 5281, 5282, 6515, 5287, 5288, and 5289, respectively;
(v) a HC CDR1, a HC CDR2, a HC CDR3, a LC CDR1, a LC CDR2 and an LC CDR3 comprising the amino acid sequences of SEQ ID NO: 5281, 5282, 6530, 5287, 5288, and 5289, respectively; or
(vi) a HC CDR1, a HC CDR2, a HC CDR3, a LC CDR1, a LC CDR2 and an LC CDR3 comprising the amino acid sequences of SEQ ID NO: 5281, 5282, 6530, 5287, 5288, and 5289, respectively.

23. The AAV particle of any one the preceding claims wherein the encoded antibody molecule comprises:
(i) a heavy chain variable region (VH) comprising an amino acid sequence of any of the VH sequences of Table 3A-3C, or a sequence having at least 90%, 95%, 96%, 97%, 98%, or 99% sequence identity to any of the VH sequences of Table 3A-3C; and/or a light chain variable region (VL) comprising an amino acid sequence of any of the VL sequences of Table 3A-3C, or a sequence having at least 90%, 95%, 96%, 97%, 98%, or 99% sequence identity to any of the VL sequences of Table 3A-3C;
(ii) a VH comprising an amino acid selected from SEQ ID NO: 5001, 5367, 5172, 5106, 5010, 5069, 5192, 5224, 5090, 5110, 5254, 5258, 5130, 5262, 5270, 5326, 6511, 6516, 6521, 6526, 6531, 6536, 6539, 6542, 6545, or 6548, or a sequence at least about 90%, 92%, 95%, 97%, 98%, or 99% identical thereto;
(iii) a VH comprising the amino acid sequence of SEQ ID NO: 5270, 6511, 6516, 6521, 6526 or 6531, or a sequence at least about 90%, 92%, 95%, 97%, 98%, or 99% identical thereto; and/or a light chain variable region comprising the amino acid sequence of SEQ ID NO: 5274, or a sequence at least about 90%, 92%, 95%, 97%, 98%, or 99% identical thereto;
(iv) a VH comprising the amino acid sequence of SEQ ID NO: 5270, or a sequence at least about 90%, 92%, 95%, 97%, 98%, or 99% identical thereto; and/or a light chain variable region comprising the amino acid sequence of SEQ ID NO: 5274, or a sequence at least about 90%, 92%, 95%, 97%, 98%, or 99% identical thereto;
(v) a VH comprising the amino acid sequence of SEQ ID NO: 5016, or a sequence at least about 90%, 92%, 95%, 97%, 98%, or 99% identical thereto; and/or a light chain variable region comprising the amino acid sequence of SEQ ID NO: 5020, or a sequence at least about 90%, 92%, 95%, 97%, 98%, or 99% identical thereto; or
(vi) a VH comprising the amino acid sequence of SEQ ID NO: 5172, or a sequence at least about 90%, 92%, 95%, 97%, 98%, or 99% identical thereto; and/or a light chain variable region comprising the amino acid sequence of SEQ ID NO: 5176, or a sequence at least about 90%, 92%, 95%, 97%, 98%, or 99% identical thereto.

24. The AAV particle of any one of the preceding claims, wherein the encoded antibody molecule comprises:
(i) a heavy chain constant region comprising an amino acid sequence of any of the heavy chain constant region sequences of Table 3A-3C or 20, or a sequence having at least 80% (e.g., 85, 90, 95, 96, 97, 98, or 99%) sequence identity to the heavy chain constant region sequences of Table 3A-3C; and/or
(ii) a light chain constant region (CL) comprising an amino acid sequence of any of the CL sequences of Table 3A-3C or 20, or a sequence having at least 80% (e.g., 85, 90, 95, 96, 97, 98, or 99%) sequence identity to any of the CL sequences of Table 3A-3C.

25. The AAV particle of any one of the preceding claims wherein the encoded antibody molecule comprises:
(i) a heavy chain comprising an amino acid sequence of any of the heavy chain sequences of Table 3A-3C, or a sequence at least 90%, 95%, 96%, 97%, 98%, or 99% identical to any of the heavy chain sequences of Table 3A-3C; and/or a light chain comprising an amino acid sequence of any of the light chain sequences of Table 3A-3C, or a sequence at least 90%, 95%, 96%, 97%, 98%, or 99% identical to any of the light chain sequences of Table 3A-3C;
(ii) a heavy chain comprising the amino acid sequence of SEQ ID NO: 5272, 7589, 6513, 6518, 6523, 6528, 6533, or a sequence at least 90%, 95%, 96%, 97%, 98%, or 99% identical thereto; and/or a light chain comprising the amino acid sequence of SEQ ID NO: 5276 or a sequence at least 90%, 95%, 96%, 97%, 98%, or 99% identical thereto;
(ii) a heavy chain comprising the amino acid sequence of SEQ ID NO: 5272, or a sequence at 90%, 95%, 96%, 97%, 98%, or 99% identical thereto; and/or a light chain comprising the amino acid sequence of SEQ ID NO: 5276, or a sequence at 90%, 95%, 96%, 97%, 98%, or 99% identical thereto;
(iii) a heavy chain comprising the amino acid sequence of SEQ ID NO: 7589, or a sequence at 90%, 95%, 96%, 97%, 98%, or 99% identical thereto; and/or a light chain comprising the amino acid sequence of SEQ ID NO: 5276, or a sequence at 90%, 95%, 96%, 97%, 98%, or 99% identical thereto.

26. The AAV particle of any one of claims 1-25, wherein the encoded antibody molecule comprises an Fc region or functional variant thereof; or an scFv and an Fc region; optionally wherein the Fc region:
(i) has reduced affinity, e.g., ablated, affinity for an Fc receptor, e.g., as compared to a reference, wherein the reference is a wild-type Fc receptor;
(ii) comprises a mutation at one, two, or all of positions I253 (e.g., I253A), H310 (e.g., H310A or H310Q), and/or H435 (e.g., H435A or H435Q), according to EU numbering;
(iii) comprises a mutation at positions I253 (e.g., I253A), H310 (e.g., H310A or H310Q), and H435 (e.g., H435A or H435Q), according to EU numbering;
(iv) comprises one, two, or all of the mutations I253A, H310A, and/or H435A, according to EU numbering;
(v) comprises one, two, or all of A at position 253, A at position 310, and/or A at position 435, according to EU numbering;
(vi) comprises the mutations I253A, H310A, and H435A, according to EU numbering;
(vii) comprises A at position 253, A at position 310, and A at position 435, according to EU numbering;
(viii) has reduced effector function (e.g., reduced ADCC), compared to a reference wherein the reference is a wild-type Fc receptor;
(ix) comprises a mutation at one, two, three, four, or all of positions L235 (e.g., L235V), F243 (e.g., F243L), R292 (e.g., R292P), Y300 (e.g., Y300L), and/or P396 (e.g., P396L), numbered according to the EU index as in Kabat;
(x) comprises a mutation at positions L235 (e.g., L235V), F243 (e.g., F243L), R292 (e.g., R292P), Y300 (e.g., Y300L), and P396 (e.g., P396L), numbered according to the EU index as in Kabat;
(xi) comprises one, two, three, four, or all of the mutations L235V, F243L, R292P, Y300L, and/or P396L, numbered according to the EU index as in Kabat;
(xii) comprises one, two, three, four, or all of V at position 235, L at position 243, P at position 292, L at position 300, and/or L at position 396, numbered according to the EU index as in Kabat;
(xiii) comprises the mutations L235V, F243L, R292P, Y300L, and P396L, numbered according to the EU index as in Kabat;
(xiv) comprises V at position 235, L at position 243, P at position 292, L at position 300, and L at position 396, numbered according to the EU index as in Kabat;
(xv) comprises a mutation at L235 (e.g., L235V), F243 (e.g., F243L), R292 (e.g., R292P), Y300 (e.g., Y300L), P396 (e.g., P396L), I253 (e.g., I253A), H310 (e.g., H310A or H310Q), and H435 (e.g., H435A or H435Q), numbered according to the EU index as in Kabat;
(xvi) comprises the mutations L235V, F243L, R292P, Y300L, P396L, I253A, H310A, and H435A, numbered according to the EU index as in Kabat; or
(xvii) comprises V at position 235, L at position 243, P at position 292, L at position 300, L at position 396, A at position 253, A at position 310, and A at position 435, numbered according to the EU index as in Kabat.

27. The AAV particle of claim 25 or 26, wherein:
(i) the nucleotide sequence encoding the VH comprises any one of the nucleotide sequences encoding a VH of Table 3A-3C, or a sequence at least 90%, 95%, 96%, 97%, 98%, or 99% identical thereto; and/or the nucleotide sequence encoding the VL comprises any one of the nucleotide sequences encoding a VL of Table 3A-3C, or a sequence at least 90%, 95%, 96%, 97%, 98%, or 99% identical thereto;
(ii) the nucleotide sequence encoding the heavy chain comprises any one of the nucleotide sequences encoding a heavy chain of Table 3A-3C, or a sequence at least 90%, 95%, 96%, 97%, 98%, or 99% identical thereto; and/or the nucleotide sequence encoding the light chain comprises any one of the nucleotide sequences encoding a light chain of Table 3A-3C, or a sequence at least 90%, 95%, 96%, 97%, 98%, or 99% identical thereto;
(iii) the nucleotide sequence encoding the VH comprises SEQ ID NO: 5269, or a nucleotide sequence at least 90%, 95%, 96%, 97%, 98%, or 99% identical thereto; and/or the nucleotide sequence encoding the VL comprises the nucleotide sequence of SEQ ID NO: 5273 or 5245, or a nucleotide sequence at least 90%, 95%, 96%, 97%, 98%, or 99% identical thereto;
(iv) the nucleotide sequence encoding the VH comprises SEQ ID NO: 5109, or a nucleotide sequence at least 90%, 95%, 96%, 97%, 98%, or 99% identical thereto; and/or the nucleotide sequence encoding the VL comprises the nucleotide sequence of SEQ ID NO: 5113, or a nucleotide sequence at least 90%, 95%, 96%, 97%, 98%, or 99% identical thereto;
(v) the nucleotide sequence encoding the VH comprises SEQ ID NO: 5002, or a nucleotide sequence at least 90%, 95%, 96%, 97%, 98%, or 99% identical thereto; and/or the nucleotide sequence encoding the VL comprises the nucleotide sequence of SEQ ID NO: 5005, or a nucleotide sequence at least 90%, 95%, 96%, 97%, 98%, or 99% identical thereto;
(vi) the nucleotide sequence encoding the VH comprises SEQ ID NO: 5261, or a nucleotide sequence at least 90%, 95%, 96%, 97%, 98%, or 99% identical thereto; and/or the nucleotide sequence encoding the VL comprises the nucleotide sequence of SEQ ID NO: 5265, or a nucleotide sequence at least 90%, 95%, 96%, 97%, 98%, or 99% identical thereto;
(vii) the nucleotide sequence encoding the heavy chain comprises SEQ ID NO: 5271, 5244, or 7590, or a nucleotide sequence at least 90%, 95%, 96%, 97%, 98%, or 99% identical thereto; and/or the nucleotide sequence encoding the light chain comprises the nucleotide sequence of SEQ ID NO: 5275 or 5246, or a nucleotide sequence at least 90%, 95%, 96%, 97%, 98%, or 99% identical thereto;
(viii) the nucleotide sequence encoding the heavy chain comprises SEQ ID NO: 7590, or a nucleotide sequence at least 90%, 95%, 96%, 97%, 98%, or 99% identical thereto; and/or the nucleotide sequence encoding the light chain comprises the nucleotide sequence of SEQ ID NO: 5246, or a nucleotide sequence at least 90%, 95%, 96%, 97%, 98%, or 99% identical thereto;
(ix) the nucleotide sequence encoding the heavy chain comprises SEQ ID NO: 5271, or a nucleotide sequence at least 90%, 95%, 96%, 97%, 98%, or 99% identical thereto; and/or the nucleotide sequence encoding the light chain comprises the nucleotide sequence of SEQ ID NO: 5275, or a nucleotide sequence at least 90%, 95%, 96%, 97%, 98%, or 99% identical thereto; or
(x) the nucleotide sequence encoding the heavy chain comprises SEQ ID NO: 5244, or a nucleotide sequence at least 90%, 95%, 96%, 97%, 98%, or 99% identical thereto; and/or the nucleotide sequence encoding the light chain comprises the nucleotide sequence of SEQ ID NO: 5246, or a nucleotide sequence at least 90%, 95%, 96%, 97%, 98%, or 99% identical thereto.

28. The AAV particle of any one of claims 1-27, wherein the encoded antibody molecule is a full-length antibody, a bispecific antibody, a Fab, a F(ab′)2, a Fv, a single chain Fv fragment (scFv), single domain antibody, or a camelid antibody.

29. The AAV particle of any one of any one of claims 1-28, wherein the encoded antibody molecule is:
(i) a multispecific antibody molecule, e.g., a bispecific antibody molecule; and/or
(ii) a bispecific, e.g., biparatopic, antibody molecule;
optionally wherein the encoded bispecific, e.g., biparatopic, antibody molecule comprises at least two antigen binding domains for two different domains of HER2, further optionally wherein the encoded bispecific, e.g., biparatopic, antibody molecule comprises:
(a) a first antigen binding domain that binds domain I of HER2 and a second antigen binding domain that binds domain IV of HER2;
(b) a first antigen binding domain that binds domain II of HER2 and a second antigen binding domain that binds domain IV of HER2;
(c) a first antigen binding domain that binds domain III of HER2 and a second antigen binding domain that binds domain IV of HER2;
(d) a first antigen binding domain that binds domain I of HER2 and a second antigen binding domain that binds domain II of HER2;
(e) a first antigen binding domain that binds domain I of HER2 and a second antigen binding domain that binds domain III of HER2; or
(f) a first antigen binding domain that binds domain II of HER2 and a second antigen binding domain that binds domain III of HER2.

30. The AAV particle of any one of claims 20-29, wherein:
(i) the nucleic acid further encodes a signal sequence, optionally wherein the signal sequence comprises a nucleotide sequence of any of the signal sequences listed in Table 11A, or a nucleotide sequence with at least 95% sequence identity thereto, optionally wherein the nucleic acid encodes a first signal sequence present 5′ relative to the encoded VH and a second signal sequence present 5′ relative to the encoded VL;
(ii) the sequences of the encoded VH and VL are connected directly, e.g., without a linker;
(iii) the sequences of the encoded VH and VL are connected via a linker;
(iv) the sequences of the encoded heavy chain and light chain are connected directly, e.g., without a linker; or
(v) the sequences of the encoded heavy chain and light chain are connected via a linker.

31. The AAV particle of any one the preceding claims, further comprising a genetic element comprising a promoter operably linked to the nucleic acid encoding the antibody molecule.

32. The AAV particle of claim 31, wherein:
(i) the promoter is a ubiquitous promoter or a tissue specific promoter;
(ii) the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase 11 (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), or a fragment, e.g., a truncation, or a functional variant thereof;
(iii) the promoter comprises a nucleotide sequence chosen from any one of SEQ ID NOs: 6563-6572, 6593-6594, or 4599, or a nucleotide sequence with at least 95% sequence identity thereto;
(iv) the promoter is a CBA promoter, optionally comprising the nucleotide sequence of SEQ ID NO: 6566 or a nucleotide sequence with at least 95% sequence identity thereto;
(v) the promoter is a GFAP promoter, optionally comprising the nucleotide sequence of SEQ ID NO: 6568, or a nucleotide sequence with at least 95% sequence identity thereto.

33. The AAV particle of claim 31 or 32, wherein the genetic element further comprises:
(i) an inverted terminal repeat (ITR) sequence, optionally wherein the ITR sequence is positioned 5′ relative to the encoded transgene and/or ITR sequence is positioned 3′ relative to the encoded transgene;
(ii) a polyadenylation (polyA) signal region, optionally wherein the polyA signal region comprises the nucleotide sequence of any of SEQ ID NOs: 6590-6592, or a nucleotide sequence with at least 95% sequence identity to any of SEQ ID NOs: 6590-6592;
(iii) an intron region, optionally wherein the intron region comprises a nucleotide sequence of any one of SEQ ID NOs: 6578-6588, 6595, 6607, 6608, or 6609, or a nucleotide sequence with at least 95% identity thereto;
(iv) an exon region, optionally wherein the exon region comprises a nucleotide sequence of any of SEQ ID NOs: 6573-6577, or a sequence with at least 95% sequence identity thereto;
(v) a Kozak sequence; and/or
(vi) a miR binding site, e.g., a miR binding site that modulates, e.g., reduces, expression of the antibody molecule encoded by the genetic element in a cell or tissue where the corresponding miRNA is expressed.

34. The AAV particle of any of one of claims 30-33, wherein the genetic element comprises the nucleotide sequence of any of SEQ ID NOs: 5163-5179 5185-5190, 5343, 5374, 5375, 6500, 6501, 6502, 6503, 6504, 6505, 6506, 6507, 6508, or 6509 or a sequence with at least 95% sequence identity thereto.

35. The AAV particle of any one of claims 30-34, wherein the genetic element further comprises a nucleic acid encoding the AAV capsid variant and/or a Rep protein, e.g., a non-structural protein, wherein the Rep protein comprises a Rep78 protein, a Rep68, Rep52 protein, and/or a Rep40 protein (e.g., a Rep52 protein and/or Rep78 protein), optionally wherein the Rep78 protein, the Rep68 protein, the Rep52 protein, and/or the Rep40 protein are encoded by at least one Rep gene.

36. A cell comprising the AAV particle of any one of claim 1-35.

37. A method of making the AAV particle of any one of claims 1-35, the method comprising
(i) providing a host cell a the genetic element; and
(ii) incubating the host cell under conditions suitable to enclose the genetic element in an AAV capsid variant;
thereby making the isolated AAV particle.

38. A pharmaceutical composition comprising an AAV particle of any one of claims 1-35, and a pharmaceutically acceptable excipient.

39. A method of delivering an exogenous antibody molecule that binds to HER2/neu, to a subject, comprising administering an effective amount of the AAV particle of any one of claims 1-35 or the pharmaceutical composition of claim 38.

40. The method of claim 39, wherein:
(i) the subject has, has been diagnosed with having, or is at risk of having a disease associated with expression of HER2/neu; and/or
(ii) the subject has, has been diagnosed with having, or is at risk of having a cancer expressing HER2/neu.

41. A method of treating a subject having or diagnosed with having cancer expressing HER2/neu, comprising administering to the subject an effective amount of the AAV particle of any one of claims 1-35 or the pharmaceutical composition of claim 38.

42. The method of claim 40 or 41, wherein the disease associated with HER2/neu expression is a HER2/neu-positive solid tumor, optionally wherein, the HER2/neu positive tumor is metastatic and/or has metastasized to the central nervous system (CNS).

43. The method of any one of claims 40-42, wherein the HER/neu positive cancer is breast cancer, gastric cancer, gastroesophageal junction cancer, colorectal cancer, lung cancer (e.g., non-small cell lung carcinoma), pancreatic cancer, bladder cancer, salivary duct cancer, ovarian cancer (e.g., epithelial ovarian cancer), endometrial cancer, prostate cancer, bone cancer and brain cancer.

44. The method of any one of claims 39-43, wherein the subject has:
(i) previously undergone localized therapy for a HER2/neu-positive solid tumor;
(ii) previously undergone surgical resection of a HER2/neu-positive solid tumor;
(iii) previously undergone radiotherapy for a HER2/neu-positive solid tumor; and/or
(iv) previously undergone immunotherapy and/or chemotherapy for a HER2/neu-positive solid tumor.

45. The method of any one of claims 39-44, wherein the AAV particle or pharmaceutical composition is administered to the subject intravenously, intramuscularly, intratumorally, intracerebrally, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM).

46. The method of any one of claims 39-45, wherein the AAV particle or pharmaceutical composition is administered to the subject:
(i) prior to, concurrently with, or post a surgical resection of a HER2/neu-positive solid tumor;
(ii) to a site of surgical resection of a HER2/neu-positive solid tumor in the subject; and/or
(iii) to the area around a site of surgical resection of a HER2/neu-positive solid tumor, e.g., the margins of the tumor, in the subject.

47. The method of any one of claims 39-46, further comprising administration of an additional therapeutic agent and/or therapy suitable for treatment or prevention of a disorder associated with HER2/neu expression optionally wherein the additional therapeutic agent comprises:
(i) trastuzumab, pertuzumab, a chemotherapeutic agent, or a combination thereof;
(ii) trastuzumab emtansine; and/or
(iii) trastuzumab, tucatinib, capecitabine, Fam-trastuzumab deruxtecan-nxki, Lapatanib/capecitabine, Lapatanib, Margetuximab, a chemotherapeutic agent, Neratanib/capecitabine, or a combination thereof.

48. The AAV particle of any one of claims 1-35 or the pharmaceutical composition of claim 38, for use in the treatment of a disease associated with expression of HER2/neu or a cancer expressing HER2/neu.

49. Use of the AAV particle of any one of claims 1-35 or the pharmaceutical composition of claim 38, in the manufacture of a medicament.

50. Use of the AAV particle of any one of claims 1-35 or the pharmaceutical composition of claim 38, in the manufacture of a medicament in the manufacture of a medicament for treating a disease associated with expression of HER2/neu or a cancer expressing HER2/neu.
